(19) United States
(12) Patent Application Publication
(10) Pub. No.: US 2026/0165023 A1
(43) Pub. Date: Jun. 11, 2026

(54) HETEROCYCLIC COMPOUND, COMPOSITION FOR ORGANIC LAYER INCLUDING THE SAME AND LIGHT EMITTING DEVICE INCLUDING THE SAME

(71) Applicant: LT MATERIALS CO., LTD., Gyeonggi-do (KR)

(72) Inventors: Ha Neul LEE, Gyeonggi-do (KR); Jun Tae MO, Gyeonggi-do (KR); Dong Jun KIM, Gyeonggi-do (KR); Dae Hyuk CHOI, Gyeonggi-do (KR)

(21) Appl. No.: 19/410,257

(22) Filed: Dec. 5, 2025

(30) Foreign Application Priority Data
Dec. 6, 2024 (KR) 10-2024-0180578

(51) Int. Cl. H10K 85/60; C07D 307/77; C07D 405/14; C07D 409/14; C09K 11/02; H10K 50/12

(52) U.S. Cl. CPC H10K 85/636; C07D 307/77; C07D 405/14; C07D 409/14; C09K 11/02; H10K 85/633; H10K 50/12; H10K 85/615; H10K 85/626; H10K 85/654; H10K 85/657; H10K 85/6572; H10K 85/6574; H10K 85/6576

(57) ABSTRACT

A heterocyclic compound is represented by Formula 1. A light-emitting device includes a first electrode, a second electrode which is disposed on the first electrode, and one or more organic layers which are interposed between the first electrode and the second electrode. The one or more of the organic layers include the heterocyclic compound.

### CROSS-REFERENCE TO RELATED APPLICATION AND CLAIM OF PRIORITY

This application claims the benefit under 35 U.S.C. § 119 of Korean Patent Application No. 10-2024-0180578 filed on Dec. 6, 2024, in the Korean Intellectual Property Office, the entire disclosure of which is incorporated herein by reference for all purposes.

### BACKGROUND

### 1. Field of the Invention

The present disclosure relates to a heterocyclic compound and a light-emitting device including the same.

### 2. Description of the Related Art

An organic light-emitting display (OLED) device includes organic light-emitting devices having self-luminescent properties. Since the organic light-emitting display device does not require a separate light source, it may provide a wide viewing angle and a fast response speed, and may enhance contrast and brightness.

In the organic light-emitting device, an organic light-emitting layer is formed for each pixel, and the organic light-emitting layer may be interposed between opposing electrodes. Holes and electrons injected from the respective electrodes recombine in the organic light-emitting layer to generate excitons, and light may be generated through the energy release of the excitons.

Research is being conducted on materials applicable to the organic light-emitting layer to achieve high efficiency and long life organic light-emitting devices.

### SUMMARY

An object of the present disclosure is to provide a heterocyclic compound.

Another object of the present disclosure is to provide a light-emitting device including the heterocyclic compound.

A heterocyclic compound according to the present disclosure is represented by Formula 1 below.

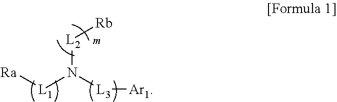

In Formula 1, Rₐ and R_{b} are each independently represented by Formula 2 below,

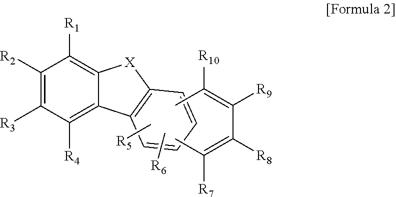

In Formula 2, one of R₁ to R₁₀ is -(L₄)_{q}-Ar₂.

Another represents a bonding site with L₁ or L₂ of Formula 1.

The others are each independently selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups are linked to form a substituted or unsubstituted 5- to 9-membered saturated or unsaturated ring. R, R′ and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

X is O or S.

In Formulae 1 and 2, L₁ to L₄ are each independently a direct linkage, a substituted or unsubstituted arylene group having 6 to 60 carbon atoms, or a substituted or unsubstituted heteroarylene group having 2 to 60 carbon atoms.

Ar₁ and Ar₂ are each independently a substituted or unsubstituted aryl group having 6 to 60 carbon atoms, or a substituted or unsubstituted heteroaryl group having 2 to 60 carbon atoms.

n, m, p, and q are each independently 0 or an integer of 1 to 5, and when n, m, p, and q are each 2 or more, a plurality of L₁ groups to L₄ groups are each the same or different.

A light-emitting device according to the present disclosure includes: a first electrode; a second electrode disposed on the first electrode; and one or more organic layers interposed between the first electrode and the second electrode, wherein one or more of the organic layers includes the heterocyclic compound.

A composition for an organic layer of an organic light-emitting device according to the present disclosure includes: the heterocyclic compound represented by Formula 1; and a compound represented by Formula 3 below.

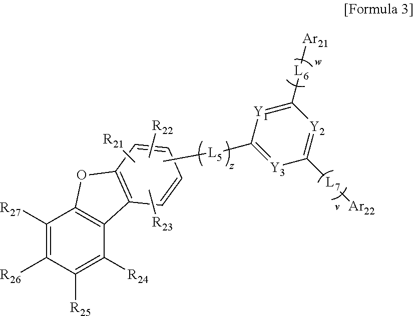

In Formula 3, any one of R₂₁ to R₂₇ is a substituted or unsubstituted aryl group having 6 to 60 carbon atoms, or a substituted or unsubstituted heteroaryl group having 2 to 60 carbon atoms.

The others of R₂₁ to R₂₇ are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a phosphine oxide group; and a silyl group; or two or more adjacent groups are linked to form a substituted or unsubstituted aromatic hydrocarbon ring having 6 to 60 carbon atoms, or a substituted or unsubstituted heterocycle having 2 to 60 carbon atoms.

L₅, L₆ and L₇ are each independently a direct linkage, a substituted or unsubstituted arylene group having 6 to 60 carbon atoms, or a substituted or unsubstituted heteroarylene group having 2 to 60 carbon atoms.

w, v and z are each independently 0 or an integer of 1 to 5.

When w, v, and z are integers of 2 to 5, a plurality of L₅ groups, L₆ groups, and L₇ groups are each the same or different.

Y₁ to Y₃ are each independently CH, nitrogen (N), oxygen (O), or sulfur (S), and at least two of Y₁ to Y₃ are nitrogen.

Ar₂₁ and Ar₂₂ are each independently a substituted or unsubstituted aryl group having 6 to 60 carbon atoms, or a substituted or unsubstituted heteroaryl group having 2 to 60 carbon atoms.

According to exemplary embodiments, the light-emitting device may include a heterocyclic compound of one embodiment. The above-described heterocyclic compound may function, for example, as a host material to control the energy band gap and energy levels of the light-emitting layer. Therefore, the light-emitting efficiency and lifetime characteristics of the light-emitting device may be improved by the above-described heterocyclic compound.

### BRIEF DESCRIPTION OF THE DRAWINGS

The above and other objects, features and advantages of the present disclosure will be more clearly understood from the following detailed description taken in conjunction with the accompanying drawings, in which:

FIGS. 1 to 3 are schematic cross-sectional views of light-emitting devices according to embodiments of the present disclosure, respectively.

### DETAILED DESCRIPTION

Hereinafter, exemplary embodiments of the present disclosure will be described in detail with reference to the accompanying drawings. However, these embodiments are merely illustrative, and the present disclosure is not limited to the specific embodiments described as examples.

As used herein, when a portion is described to “include” a component, unless otherwise specified, it means that the portion does not exclude other components and may further include other components.

As used herein, the * symbol in a chemical formula represents a bonding site.

As used herein, “substitution” means that any hydrogen atom bonded to a carbon atom in a compound is replaced with another substituent. The position of substitution is not limited as long as the substituent is at a substitutable position. If two or more substitutions are made, the two or more substituents may be the same or different.

As used herein, “substituted or unsubstituted” means that at least one hydrogen atom of a compound is unsubstituted or substituted with one or more substituents selected from the group consisting of substituents, or is substituted with a substituent in which two or more of the following substituents are linked: deuterium; a halogen group; —CN; an alkyl group having 1 to 60 carbon atoms; an alkenyl group having 2 to 60 carbon atoms; an alkynyl group having 2 to 60 carbon atoms; a haloalkyl group having 1 to 60 carbon atoms; an alkoxy group having 1 to 60 carbon atoms; an aryloxy group having 6 to 60 carbon atoms; an alkylthio group having 1 to 60 carbon atoms; an arylthio group having 6 to 60 carbon atoms; an alkylsulfinyl group having 1 to 60 carbon atoms; an arylsulfinyl group having 6 to 60 carbon atoms; a cycloalkyl group having 3 to 60 carbon atoms; a heterocycloalkyl group having 2 to 60 carbon atoms; an aryl group having 6 to 60 carbon atoms; a heteroaryl group having 2 to 60 carbon atoms; —SiRR′R″; —P(═O)RR′; and —NRR′. R, R′ and R″ are each independently a substituent including at least one of hydrogen; deuterium; a halogen group; an alkyl group; an alkenyl group; an alkoxy group; a cycloalkyl group; a heterocycloalkyl group; an aryl group; and a heteroaryl group.

As used herein, when a substituent is not indicated in a chemical formula or compound structure, it means that a hydrogen atom is bonded to the carbon atom. However, since deuterium (²H) is an isotope of hydrogen, some hydrogen atoms may be deuterium.

As used herein, when a substituent is not indicated in a chemical formula or compound structure, it may mean that all positions that can be substituted are hydrogen or deuterium. In other words, deuterium is an isotope of hydrogen, and some of the hydrogen atoms may be deuterium atoms, in which case the deuterium content may be 0% to 100%.

When a substituent is not indicated in a chemical formula or compound structure, and the deuterium content is 00%, the hydrogen content is 100%, and all substituents are hydrogen, hydrogen and deuterium may nevertheless be present together in the compound unless deuterium is explicitly excluded.

Deuterium is an isotope of hydrogen, having a nucleus composed of one proton and one neutron (a deuteron). It may be represented as hydrogen-2 and its elemental symbol may be written as D or ²H.

Isotopes are atoms with the same atomic number (Z) but different mass numbers (A). Isotopes may also be defined as atoms with the same number of protons but different numbers of neutrons.

As used herein, the content T % of a specific substituent is defined as T2/T1×100=T (%), where the total number of substituents that a basic compound may have is defined as T1, and the number of the specific substituent among them is defined as T2.

In one example, a 20% deuterium content in a phenyl group represented by.

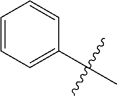 [SMILES: *c1ccccc1]

means that the total number of substituents that the phenyl group may have is 5 (T1 in the formula), and among them, the number of deuterium atoms is 1 (T2 in the formula), which may be represented as 20%. That is, cases where the deuterium content in the phenyl group is 20% may be represented by the structural formula below.

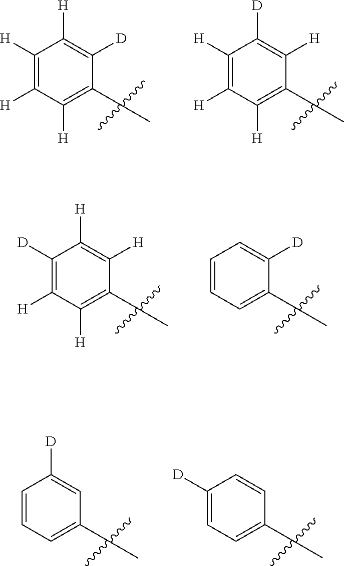 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, “a phenyl group having 0% deuterium content” may refer to a phenyl group that includes no deuterium atoms and has five hydrogen atoms.

As used herein, halogen may be fluorine, chlorine, bromine, or iodine.

As used herein, an alkyl group includes a straight or branched chain having 1 to 60 carbon atoms, and may be further substituted with one or more substituents. The alkyl group may have 1 to 60, 1 to 40, or 1 to 20 carbon atoms. Examples thereof may include a methyl group, an ethyl group, a propyl group, an n-propyl group, an isopropyl group, a butyl group, an n-butyl group, an isobutyl group, a tert-butyl group, a sec-butyl group, a 1-methylbutyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethylpropyl group, a 1,1-dimethylpropyl group, an isohexyl group, a 2-methylpentyl group, a 4-methylhexyl group, and a 5-methylhexyl group, but are not limited thereto.

As used herein, the alkenyl group includes a straight or branched chain having 2 to 60 carbon atoms, and may be further substituted with one or more substituents. The alkenyl group may have 2 to 60, 2 to 40, or 2 to 20 carbon atoms. Examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl group, a 2-phenylvinyl group, a 2,2-diphenylvinyl group, a 2-phenyl-2-(naphthyl-1-yl)vinyl group, a 2,2-bis(diphenyl)vinyl group, a stilbenyl group, and a styrenyl group, but are not limited thereto.

As used herein, an alkynyl group includes a straight or branched chain having 2 to 60 carbon atoms, and may be further substituted with one or more substituents. The alkynyl group may have 2 to 60, 2 to 40, or 2 to 20 carbon atoms.

As used herein, a haloalkyl group refers to an alkyl group substituted with a halogen group. Examples there of may include —CF₃ and —CF₂CF₃, but are not limited thereto.

As used herein, a cycloalkyl group includes a monocyclic or polycyclic group having 3 to 60 carbon atoms, and may be further substituted with one or more substituents. In this context, a polycyclic group refers to a group in which a cycloalkyl group is directly linked to or condensed with another cyclic group. The other cyclic group may be a cycloalkyl group, but may also be another type of cyclic group, such as a heterocycloalkyl group, an aryl group, or a heteroaryl group. The number of carbon atoms in the cycloalkyl group may be 3 to 60, 3 to 40, or 5 to 20. Examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, and a cyclooctyl group, but are not limited thereto.

As used herein, a heterocycloalkyl group includes a monocyclic or polycyclic group having 2 to 60 carbon atoms and including at least one heteroatom selected from O, S, Se, N and Si, and may be further substituted with one or more substituents. In this context, the term “polycyclic” refers to a group in which a heterocycloalkyl group is directly linked to or condensed with another ring group. The other ring group may be a heterocycloalkyl group, but may also be another type of ring group, such as a cycloalkyl group, an aryl group, or a heteroaryl group. The heterocycloalkyl group may have 2 to 60, 2 to 40, or 3 to 20 carbon atoms.

As used herein, an aryl group includes a monocyclic or polycyclic group having 6 to 60 carbon atoms, and may be further substituted with one or more substituents. In this context, the term “polycyclic” refers to a group in which an aryl group is directly linked to or condensed with another ring group. The other ring group may be an aryl group, but may also be another type of ring group, such as a cycloalkyl group, a heterocycloalkyl group, or a heteroaryl group. The aryl group may include a spiro ring structure. The aryl group may have 6 to 60, 6 to 40, or 6 to 25 carbon atoms. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, and condensed ring groups thereof, but are not limited thereto.

As used herein, the terphenyl group may be selected from the following structures.

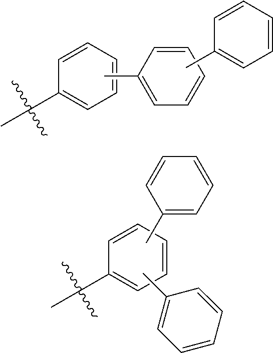

As used herein, the fluorenyl group may be substituted, and adjacent substituents may be bonded to form a ring.

The substituted fluorenyl group may be represented by the following structural formula, but is not limited thereto.

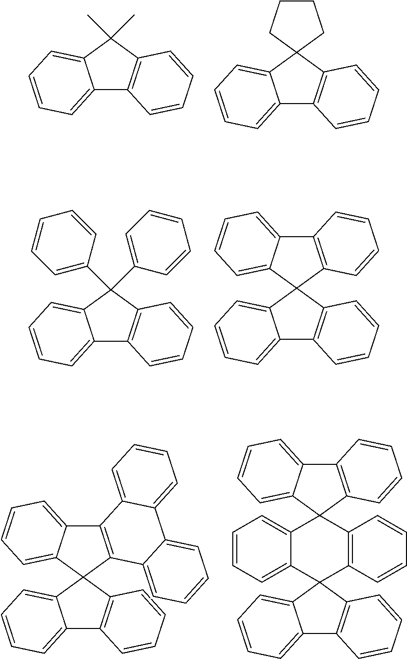

As used herein, an alkoxy group is represented by —O(R101), and R101 may be selected from the examples of the alkyl group described above.

As used herein, an aryloxy group is represented by —O(R102), and R102 may be selected from the examples of the aryl group described above.

As used herein, an alkylthio group is represented by —S(R103), and R103 may be selected from the examples of the alkyl group described above.

As used herein, an arylthio group is represented by —S(R104), and R104 may be selected from the examples of the aryl group described above.

As used herein, an alkylsulfinyl group is represented by —S(═O)₂(R105), and R105 may be selected from the examples of the alkyl group described above.

As used herein, an arylsulfinyl group is represented by —S(═O)₂(R106), and R106 may be selected from the examples of the aryl group described above.

In the present specification, a heteroaryl group includes at least one heteroatom selected from S, O, Se, N, and Si, and includes a monocyclic or polycyclic group having 2 to 60 carbon atoms, and may be further substituted by another substituent. Here, the polycyclic group refers to a group in which a heteroaryl group is directly connected to or condensed with another ring group. Here, the other ring group may be a heteroaryl group, but may also be another type of ring group, such as a cycloalkyl group, a heterocycloalkyl group, an aryl group, etc. The heteroaryl group may have 2 to 60, 2 to 40, or 3 to 25 carbon atoms. Examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, benzoxazole group, benzimidazole group, benzothiophene group, benzofuran group, dibenzothiophene group, dibenzofiran group, catbazolyl group, benzocarbazolyl group, dibenzocarbazolyl group, phenazine group, dibenzosilole group, spirobi(dibenzosilole), dihydrophenazine group, phenoxazine group, phenanthridine group, thienyl group, indolo[2,3-a]catbazolyl group, indolo[2,3-b]carbazolyl group, indoline group, 10,11-dihydro-dibenzo[b,f]azepine group, 9,10-dihydroacridine group, phenanthrazine group, phenothiathiazine group, phthalazine group, phenanthroline group, naphthobenzofuran group, naphthobenzothiophene group, benzo[c][1,2,5]thiadiazole group, Examples thereof include, but are not limited to, 2,3-dihydrobenzo[b]thiophene group, 2,3-dihydrobenzofuran group, 5,10-dihydrodibenzo[b,e][1,4]azacillin group, pyrazolo[1,5-c]quinazoline group, pyrido[1,2-b]indazole group, pyrido[1,2-a]imidazo[1,2-e]indolin group, and 5,11-dihydroindeno[1,2-b]catbazolyl group.

In this specification, when a substituent is a carbazolyl group, it means bonding to the nitrogen or carbon of the carbazolyl group.

In this specification, when a carbazolyl group is substituted, an additional substituent may be substituted on the nitrogen or carbon of the carbazolyl group.

In this specification, examples of a benzocarbazolyl group may have any of the following structures:

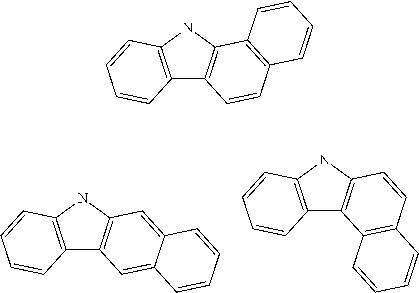

As used herein, examples of a dibenzocarbazolyl group may include any one of the following structures.

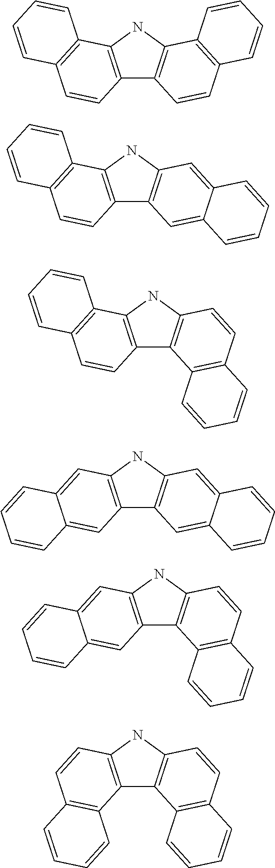

As used herein, examples of a naphthobenzofuran group may include any one of the following structures.

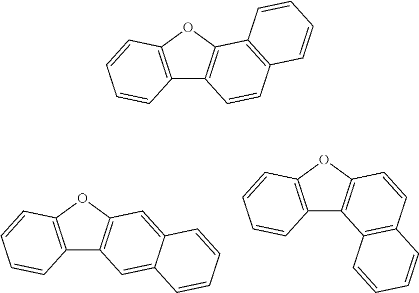

As used herein, examples of a naphthobenzothiophene group may include any one of the following structures.

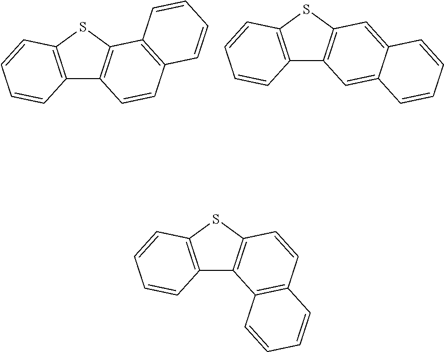

Among the substituents, —SiRR′R″ is a silyl group, which is a substituent that includes Si, and the Si atom is directly bonded as a radical. R, R′ and R″ may be the same or different and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group. Examples of the silyl group may include,

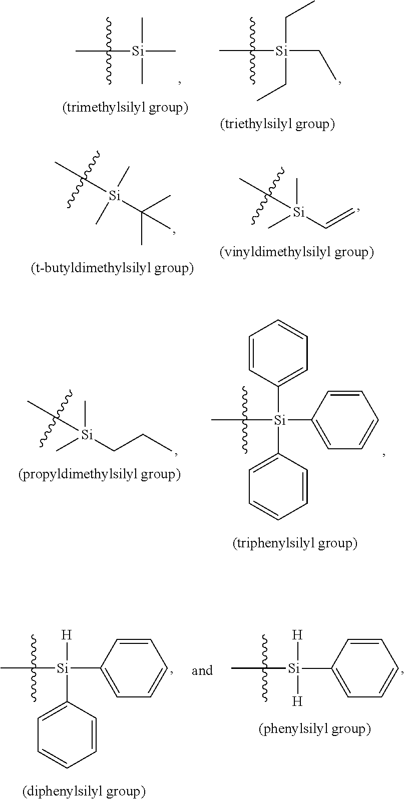

but are not limited thereto.

Among the substituents, —P(═O)RR′ is a phosphine oxide group, wherein R and R′ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group, and in particular may be an alkyl group or an aryl group. The alkyl group and aryl group may be selected from the examples described above. For example, the phosphine oxide group may include a dimethylphosphine oxide group, a diphenylphosphine oxide group, and a dinaphthylphosphine oxide group, but is not limited thereto.

Among the substituents, —NRR′ is an amine group, wherein R and R′ may be the same or different, and each independently may be a substituent including at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH, a monoalkylamine group, a monoarylamine group, a monoheteroarylamine group, a dialkylamine group, a diarylamine group, a diheteroarylamine group, an alkylarylamine group, an alkylheteroarylamine group, and an arylheteroarylamine group, and the number of carbon atoms is not particularly limited, but is preferably 1 to 30. Examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methylanthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, and a biphenyltriphenylenylamine group, but are not limited thereto.

As used herein, an arylene group may be selected from the examples of the aryl group described above, except that it is divalent.

As used herein, a heteroarylene group may be selected from the examples of the heteroaryl group described above, except that itis divalent.

As used herein, the term “adjacent” group may refer to a substituent that is substituted on an atom directly linked to the atom substituted by the substituent, a substituent that is sterically closest to the substituent, or another substituent substituted on the atom substituted by the substituent. For example, two substituents substituted at the ortho positions of a benzene ring and two substituents substituted on the same carbon atom of an aliphatic ring may be interpreted as “adjacent” groups.

Hydrocarbon rings and heterocycles that can be formed by adjacent groups may include aliphatic hydrocarbon rings, aromatic hydrocarbon rings, aliphatic heterocycles, and aromatic heterocycles, and the structures exemplified by the above-described cycloalkyl groups, aryl groups, heterocycloalkyl groups, and heteroaryl groups may apply thereto, except that they are not monovalent.

Generally, compounds bonded to hydrogen and compounds substituted with deuterium exhibit different thermodynamic behaviors. The reason is that the mass of the deuterium atom is twice that of hydrogen. Due to this difference in atomic mass, deuterium has the characteristic of having lower vibrational energy.

In addition, the single bond dissociation energy (BDE) of carbon-deuterium is higher than that of carbon-hydrogen. Therefore, structures substituted with deuterium have increased thermal stability, resulting in improved lifetime of devices utilizing them.

When a compound is deposited on a silicon wafer, substances including deuterium tend to pack more tightly at intermolecular distance. In addition, observation of the thin film surface with an atomic force microscope (AFM) reveals that thin films formed from deuterium-containing compounds are deposited with a more uniform surface and without agglomeration.

The heterocyclic compound of Formula 1 of the present disclosure has a deuterium substitution ratio of greater than 0% and not more than 100%. When deuterium is substituted, the ground state energy is lowered compared to hydrogen-substituted compounds. Furthermore, as the carbon-deuterium bond length decreases, the molecular hardcore volume decreases. This can reduce electrical polarizability and weaken intermolecular interactions, resulting in a more stable stacking structure during device fabrication.

These characteristics create an amorphous state in the thin film, thereby lowering the crystallinity. In other words, the heterocyclic compound of Formula 1 may be effective in improving the heat resistance of OLED devices, thereby enhancing their lifetime and operating characteristics.

The heterocyclic compound according to the present disclosure may be represented by Formula 1 below.

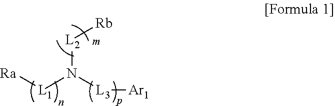

In Formula 1, L₁ to L₃ are each independently a direct linkage, a substituted or unsubstituted arylene group having 6 to 60 carbon atoms, or a substituted or unsubstituted heteroarylene group having 2 to 60 carbon atoms.

According to exemplary embodiments, L₁ to L₃ may each independently be a direct linkage, a substituted or unsubstituted arylene group having 6 to 40 carbon atoms, or a substituted or unsubstituted heteroarylene group having 2 to 40 carbon atoms. According to some embodiments, L₁ to L₃ may each independently be a direct linkage, a substituted or unsubstituted arylene group having 6 to 20 carbon atoms, or a substituted or unsubstituted heteroarylene group having 2 to 20 carbon atoms.

For example, L₁ to L₃ may each independently be a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, or a substituted or unsubstituted carbazolyl group.

For example, L₁ to L₃ may each independently be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated anthryl group, a deuterated or non-deuterated carbazolyl group.

For example, L₁ and L₂ may each be a direct linkage. In one embodiment, the heterocyclic compound may be represented by Formula 1-1 below.

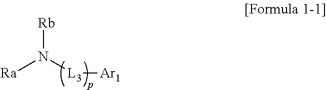

n, m, and p are each independently 0 or an integer of 1 to 5. According to exemplary embodiments, n, m, and p are each independently 0 or an integer of 1 to 3. According to some embodiments, n, m, and p are each independently 0, 1, or 2.

When n, m, and p are each 2 or more, a plurality of L₁ groups to L₃ groups may each be the same or different. For example, when n is 2 or more, two or more L₁ groups may be the same or different, when m is 2 or more, two or more L₂ groups may be the same or different, and when p is 2 or more, two or more L₃ groups may be the same or different.

In Formula 1, Ar₁ is a substituted or unsubstituted aryl group having 6 to 60 carbon atoms, or a substituted or unsubstituted heteroaryl group having 2 to 60 carbon atoms. According to exemplary embodiments, Ar₁ may be a substituted or unsubstituted aryl group having 6 to 40 carbon atoms, or a substituted or unsubstituted heteroaryl group having 2 to 40 carbon atoms. According to some embodiments, Ar₁ may be a substituted or unsubstituted aryl group having 6 to 20 carbon atoms, or a substituted or unsubstituted heteroaryl group having 2 to 20 carbon atoms.

For example, Ar₁ may be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted carbazolyl group, or a combination thereof.

For example, Ar₁ may be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated anthryl group, a deuterated or non-deuterated carbazolyl group, or a combination thereof.

In Formula 1, Rₐ and R_{b} are each independently represented by Formula 2 below.

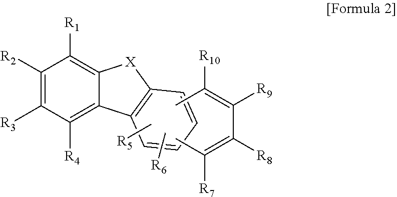

In Formula 2, one of R₁ to R₁₀ is -(L₄)_{q}-Ar₂.

L₄ is a direct linkage, a substituted or unsubstituted arylene group having 6 to 60 carbon atoms, or a substituted or unsubstituted heteroarylene group having 2 to 60 carbon atoms.

In exemplary embodiments, L₄ may be a direct linkage, a substituted or unsubstituted arylene group having 6 to 40 carbon atoms, or a substituted or unsubstituted heteroarylene group having 2 to 40 carbon atoms. In some embodiments, L₄ may be a direct linkage, a substituted or unsubstituted arylene group having 6 to 20 carbon atoms, or a substituted or unsubstituted heteroarylene group having 2 to 20 carbon atoms.

For example, L₄ may be a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, or a substituted or unsubstituted carbazolyl group.

For example, L₄ may be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated anthryl group, a deuterated or non-deuterated carbazolyl group.

q is 0 or an integer of 1 to 5. In exemplary embodiments, q is 0 or an integer of 1 to 3. In some embodiments, q is 0, 1, or 2.

When q is 2 or more, a plurality of L₄ groups are each the same or different.

Ar₂ is a substituted or unsubstituted aryl group having 6 to 60 carbon atoms, or a substituted or unsubstituted heteroaryl group having 2 to 60 carbon atoms. In exemplary embodiments, Ar₂ may be a substituted or unsubstituted aryl group having 6 to 40 carbon atoms, or a substituted or unsubstituted heteroaryl group having 2 to 40 carbon atoms. According to some embodiments, Ar₂ may be a substituted or unsubstituted aryl group having 6 to 20 carbon atoms, or a substituted or unsubstituted heteroaryl group having 2 to 20 carbon atoms.

For example, Ar₂ may be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, a substituted or unsubstituted carbazolyl group, or a combination thereof.

For example, Ar₂ may be a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated anthryl group, a deuterated or non-deuterated carbazolyl group, or a combination thereof.

In Formula 2, among R₁ to R₁₀, one of those other than -(L₄)_{q}-Ar₂ is a bonding site with L₁ or L₂ of Formula 1.

In Formula 2, among R₁ to R₁₀, the others, except those that are -(L₄)_{q}-Ar₂ and those that are bonding sites with L₁ or L₂ of Formula 1, are each independently hydrogen, deuterium, or a substituent. The term “substituent” may be selected from the group consisting of a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted 5- to 9-membered saturated or unsaturated ring.

R, R′, and R″ are each independently hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

In Formula 2, X is O or S.

According to exemplary embodiments, in Formula 1, Rₐ and R_{b} may each independently be represented by any one of Formulae 2-1 to 2-3 below.

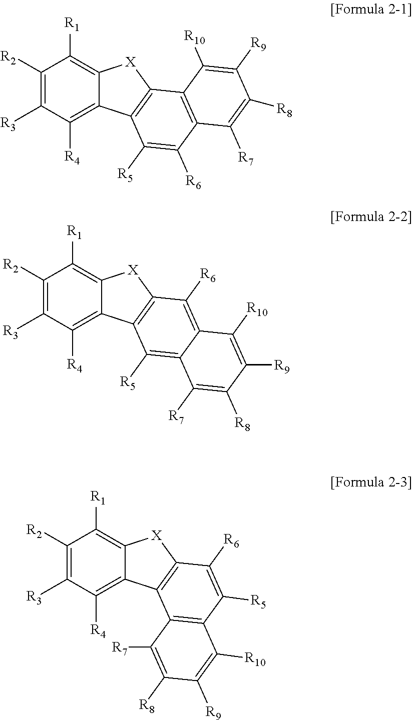

In Formulae 2-1 to 2-3, the definitions of X and R₁ to R₁₀ are the same as those described above.

According to exemplary embodiments, in Formula 1, Rₐ and R_{b} may each independently be represented by any one of Formulae 2-1-1 to 2-1-11, Formulae 2-2-1 to 2-2-6, and Formulae 2-3-1 to 2-3-5 below.

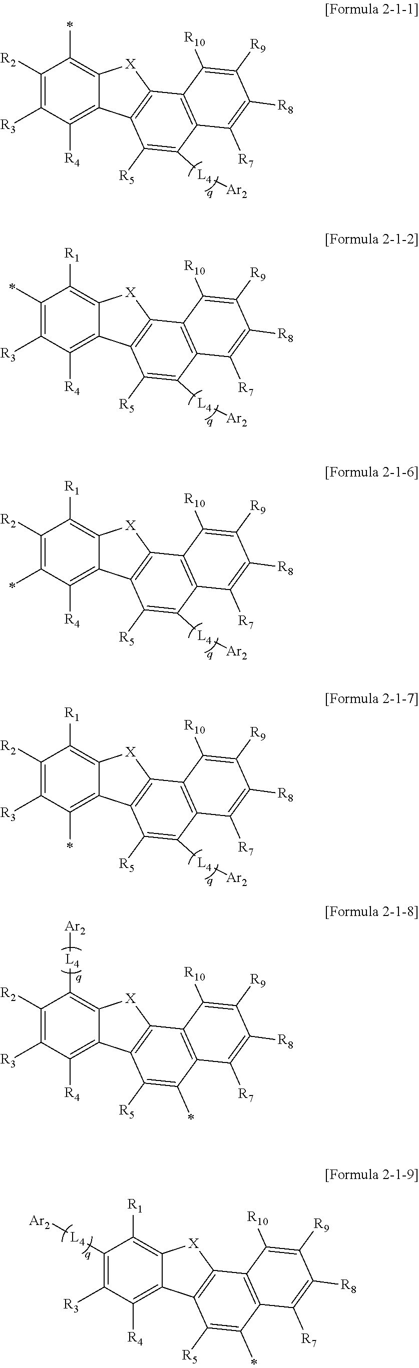

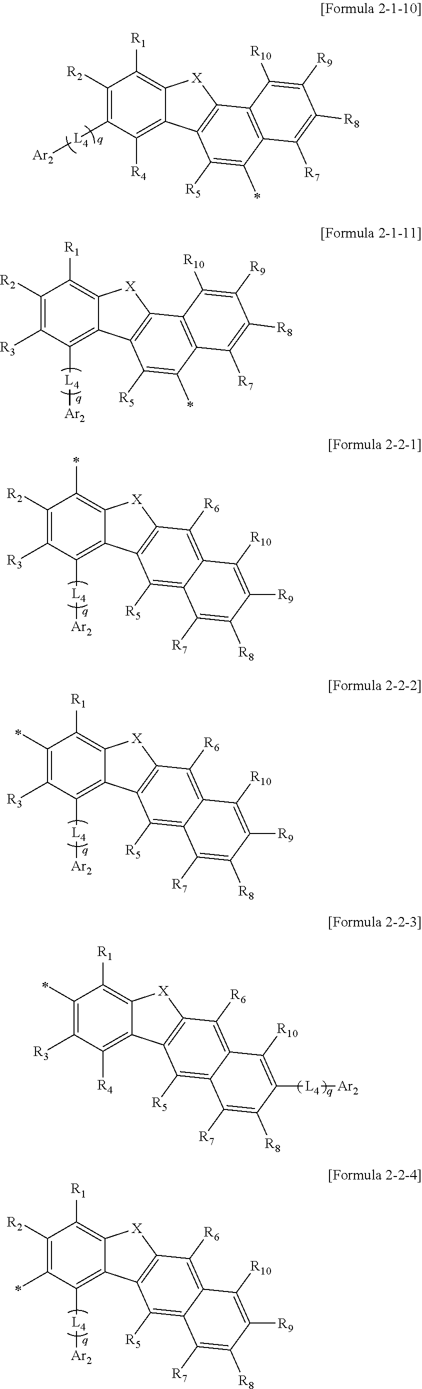

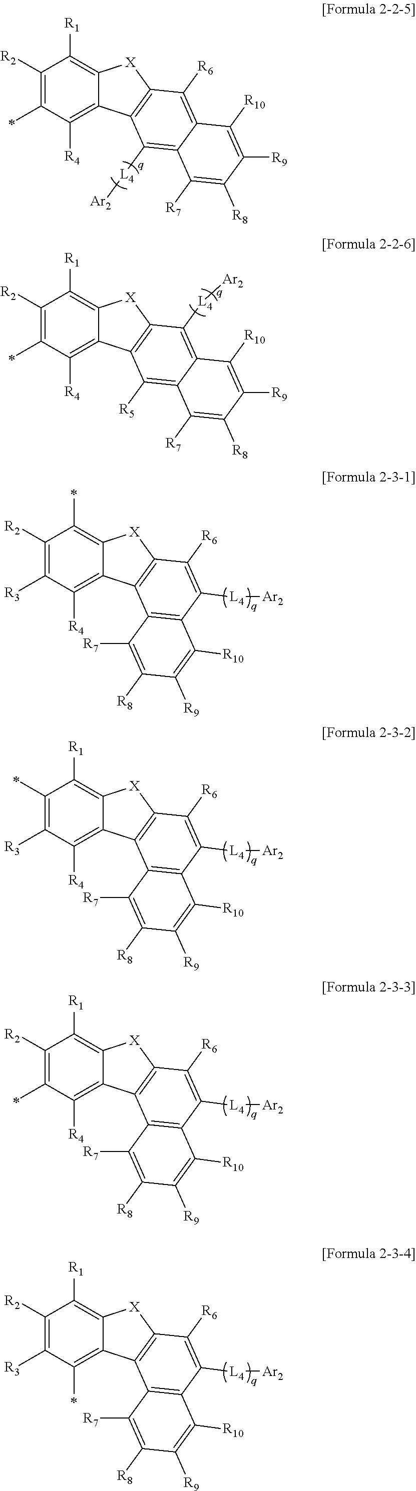

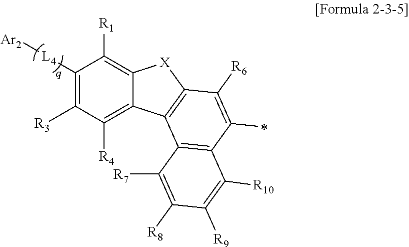

In Formulae 2-1-1 to 2-1-11, Formulae 2-2-1 to 2-2-6, and Formulae 2-3-1 to 2-3-5, * represents a bonding site. The definitions of X, R₁ to R₁₀, L₄, Ar₂, and q are the same as those described above.

According to exemplary embodiments, in Formulae 2-1-1 to 2-1-11, Formulae 2-2-1 to 2-2-6, and Formulae 2-3-1 to 2-3-5, q may independently be 0, 1, or 2.

In Formulae 2-1-1 to 2-1-11, Formulae 2-2-1 to 2-2-6, and Formulae 2-3-1 to 2-3-5, Ar₂ may independently be a substituted or unsubstituted aryl group having 6 to 40 carbon atoms, or a substituted or unsubstituted heteroaryl group having 2 to 40 carbon atoms.

According to exemplary embodiments, Ra and Rb may each be represented by any one of Formulae 2-1-1 to 2-1-11, Formulae 2-2-1 to 2-2-6, and Formulae 2-3-1 to 2-3-5, and may be the same or different.

According to some embodiments, Ra and Rb may each be represented by anyone selected from Formulae 2-1-1 to 2-1-11, Formulae 2-2-1 to 2-2-6 and Formulae 2-3-1 to 2-3-5, and Ra and Rb may have structures in which X, R₁ to R₁₀, L₄, Ar₂ and qin the selected chemical formula are each independently the same or different.

For example, Ra and Rb may each be represented by anyone selected from Formulae 2-1-1 to 2-1-11, Formulae 2-2-1 to 2-2-6, and Formulae 2-3-1 to 2-3-5, and Ra and Rb may have structures in which the degree of deuterium substitution is different in the selected chemical formula.

In one embodiment, Ra and Rb may be the same. For example, Ra and Rb may each be represented by any one selected from Formulae 2-1-1 to 2-1-11, Formulae 2-2-1 to 2-2-6, and Formulae 2-3-1 to 2-3-5, and X, R₁ to R₁₀, L₄, Ar₂, and qin the selected chemical formula may have the same structure.

The heterocyclic compound may be represented by anyone of Formulae 1-2 to 1-4 below.

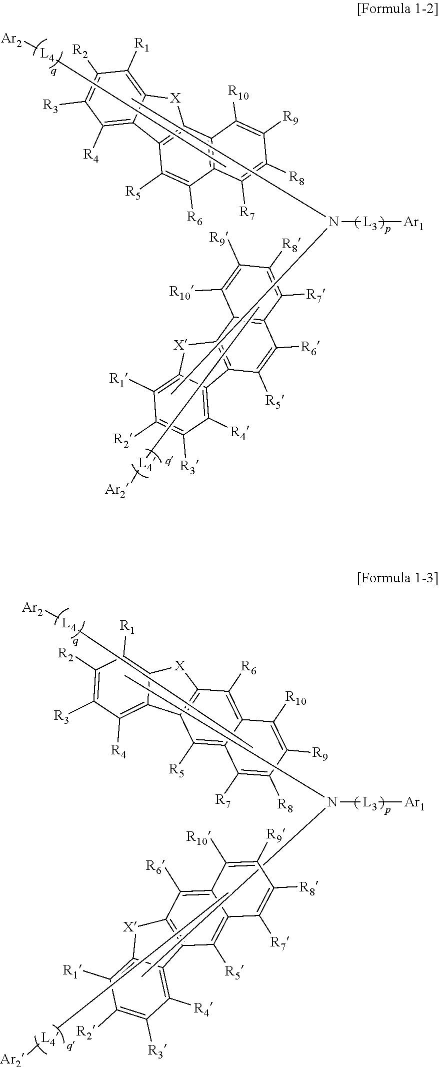

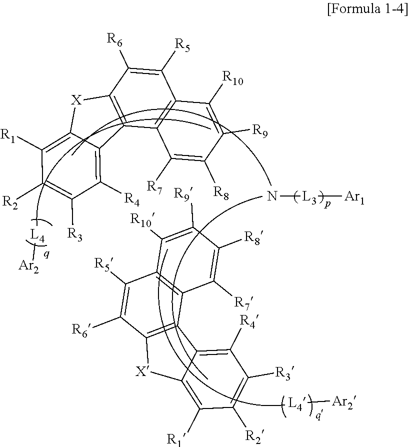

In Formulae 1-2 to 1-4, the definitions of X, L₃, L₄, Ar₁, Ar₂, p, and q may be the same as those described above.

In Formulae 1-2 to 1-4, X may be O or S, and may be the same or different from X.

In Formulae 1-2 to 1-4, one of R₁ to R₁₀ may be a bonding site with L₄, and the other may be a bonding site with N. One of R₁′ to R₁₀′ may be a bonding site with L₄′, and the other may be a bonding site with N.

In Formulae 1-2 to 1-4, the substituents other than those serving as bonding sites with L₄, L₄′, or N among R₁ to R₁₀ and R₁′ to R₁₀′ may each independently be selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted 5- to 9-membered saturated or unsaturated ring.

R, R′, and R″ are each independently hydrogen; deuterium; —CN, a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

In Formulae 1-2 to 1-4, Ar₂′ may be a substituted or unsubstituted aryl group having 6 to 60 carbon atoms, or a substituted or unsubstituted heteroaryl group having 2 to 60 carbon atoms. For example, Ar₂ may be the same or different from Ar₂.

In Formulae 1-2 to 1-4, L₄′ may be a direct linkage, a substituted or unsubstituted arylene group having 6 to 60 carbon atoms, or a substituted or unsubstituted heteroarylene group having 2 to 60 carbon atoms. For example, L₄′ may be the same or different from L₄.

In Formulae 1-2 to 1-4, q′ may be 0 or an integer of 1 to 5. For example, q′ may be 0, 1, or 2.

In Formulae 1-2 to 1-4, when q′ is 2 or more, a plurality of L₄′ groups may be the same or different.

According to exemplary embodiments, the heterocyclic compound may be represented by any one of Formulae 1-5 to 1-23 below.

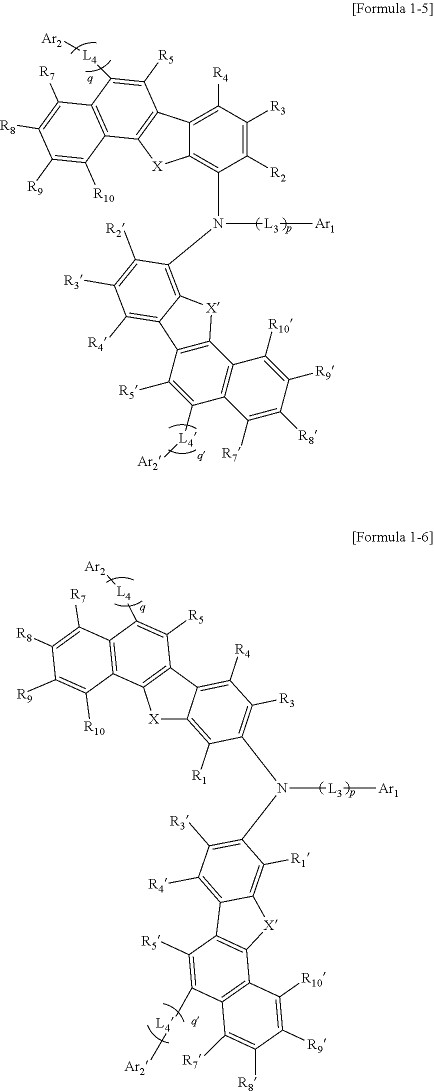

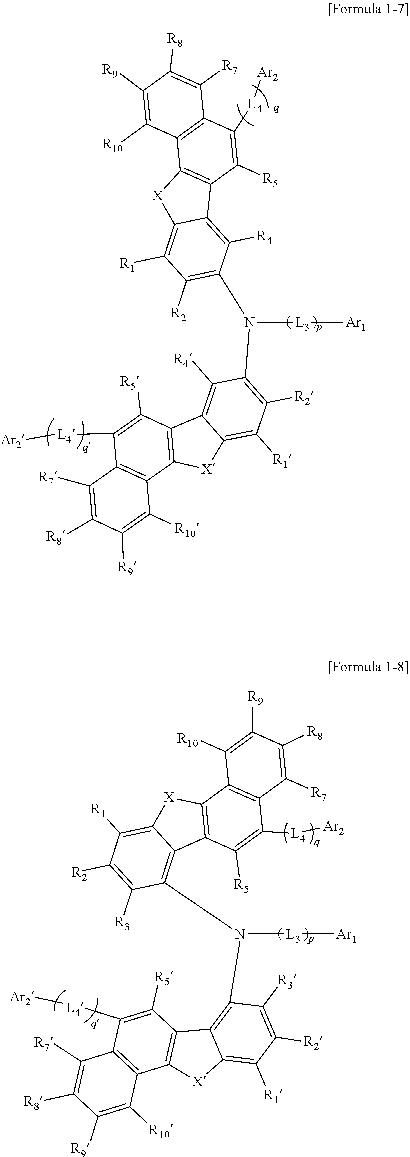

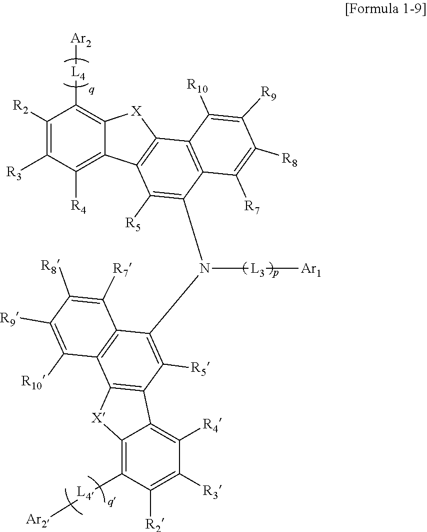

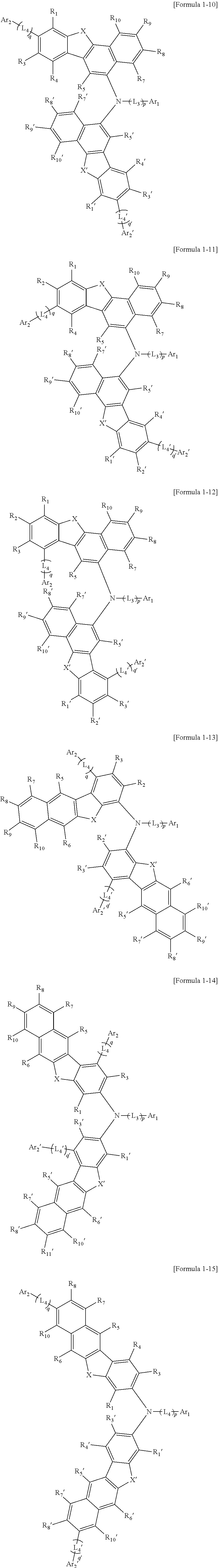

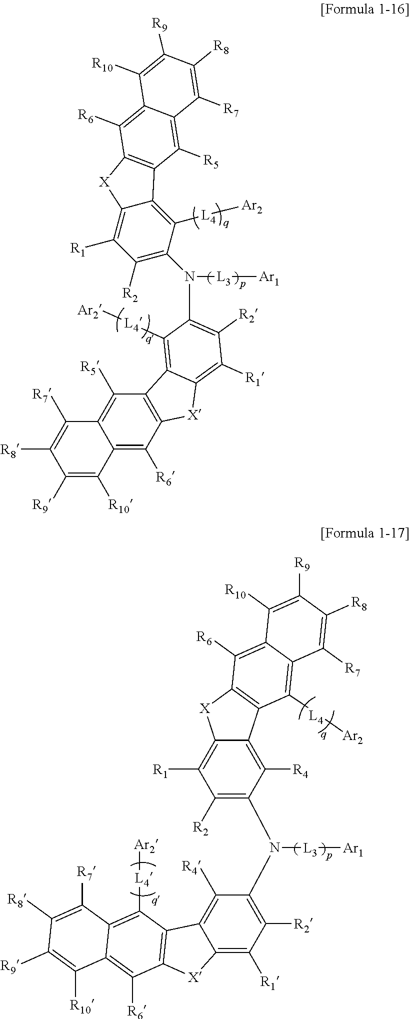

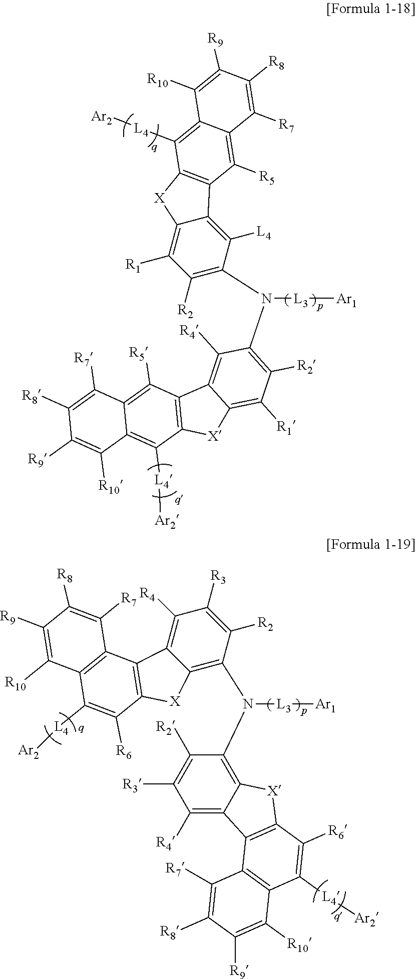

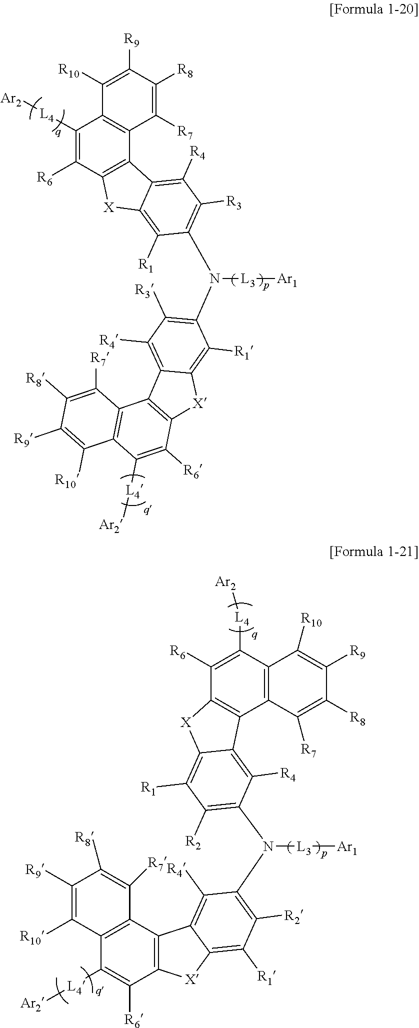

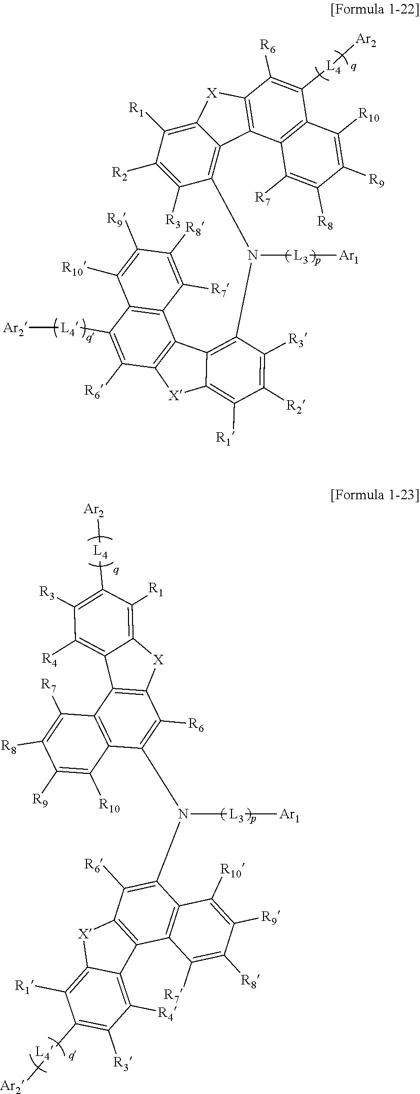

In Formulae 1-5 to 1-23, the definitions of X, R₁ to R₁₀, L₃, L₄, Ar₁, Ar₂, and p and q may be the same as those described above.

In Formulae 1-5 to 1-23, X may be O or S, and may be the same or different from X.

In Formulae 1-5 to 1-23, R₁′ to R₁₀′ may each independently be selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted 5- to 9-membered saturated or unsaturated ring.

R, R′, and R″ may each independently be hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

In Formulae 1-5 to 1-23, Ar2′ may be a substituted or unsubstituted aryl group having 6 to 60 carbon atoms, or a substituted or unsubstituted heteroaryl group having 2 to 60 carbon atoms.

In Formulae 1-5 to 1-23, L4′ may be a direct linkage, a substituted or unsubstituted arylene group having 6 to 60 carbon atoms, or a substituted or unsubstituted heteroarylene group having 2 to 60 carbon atoms.

q′ may be 0 or an integer of 1 to 5. When q′ is 2 or more, a plurality of L₄′ groups may be the same or different.

According to exemplary embodiments, the deuterium content of the compound of Formula 1 may be 0%, or greater than 0 and not more than 100%.

According to exemplary embodiments, the deuterium content of the compound of Formula 1 may be 0%, or 5% to 100%.

According to exemplary embodiments, the deuterium content of the compound of Formula 1 may be 0%, or 10% to 100%.

According to exemplary embodiments, the deuterium content of the compound of Formula 1 may be 0%, or 15% to 100%.

According to exemplary embodiments, the deuterium content of the compound of Formula 1 may be 0%, or 20% to 100%.

According to exemplary embodiments, the deuterium content of the compound of Formula 1 may be 0%, or 25% to 100%.

According to exemplary embodiments, the deuterium content of the compound of Formula 1 may be 0%, or 30% to 100%.

According to exemplary embodiments, the deuterium content of the compound of Formula 1 may be 0%, or 50% to 100%.

According to exemplary embodiments, the deuterium content of the compound of Formula 1 may be 0%, or 70% to 100%.

According to exemplary embodiments, the deuterium content of the compound of Formula 1 may be 0%, or 90% to 100%.

According to exemplary embodiments, the deuterium content of the compound of Formula 1 may be 0%.

According to exemplary embodiments, the deuterium content of the compound of Formula 1 may be 30% to 100%.

According to exemplary embodiments, the deuterium content of the compound of Formula 1 may be 50% to 100%.

According to exemplary embodiments, the deuterium content of the compound of Formula 1 may be 70% to 100%.

According to exemplary embodiments, the deuterium content of the compound of Formula 1 may be 90% to 100%.

The heterocyclic compound may be represented by any one of formulae below.

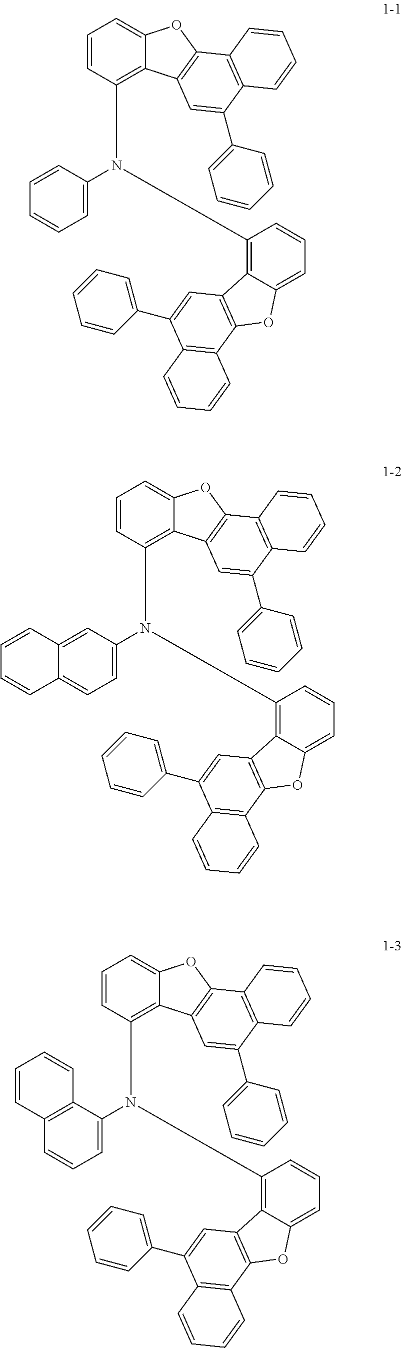 [SMILES: c1ccc(-c2cc3c(oc4cccc(N(c5ccccc5)c5cccc6oc7c8ccccc8c(-c8ccccc8)cc7c56)c43)c3ccccc23)cc1 ; c1ccc(-c2cc3c(oc4cccc(N(c5ccc6ccccc6c5)c5cccc6oc7c8ccccc8c(-c8ccccc8)cc7c56)c43)c3ccccc23)cc1 ; c1ccc(-c2cc3c(oc4cccc(N(c5cccc6ccccc56)c5cccc6oc7c8ccccc8c(-c8ccccc8)cc7c56)c43)c3ccccc23)cc1]

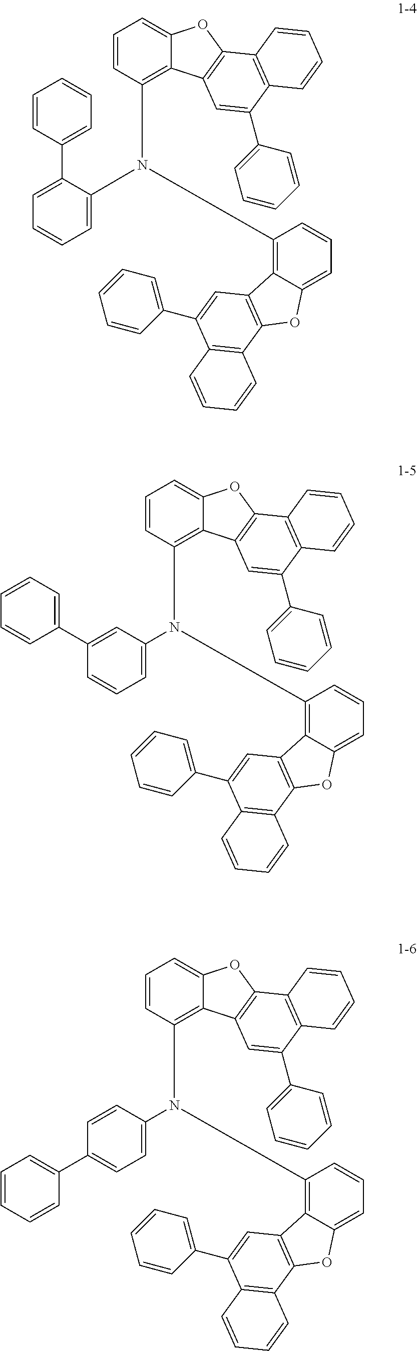 [SMILES: c1ccc(-c2ccccc2N(c2cccc3oc4c5ccccc5c(-c5ccccc5)cc4c23)c2cccc3oc4c5ccccc5c(-c5ccccc5)cc4c23)cc1 ; c1ccc(-c2cccc(N(c3cccc4oc5c6ccccc6c(-c6ccccc6)cc5c34)c3cccc4oc5c6ccccc6c(-c6ccccc6)cc5c34)c2)cc1 ; c1ccc(-c2ccc(N(c3cccc4oc5c6ccccc6c(-c6ccccc6)cc5c34)c3cccc4oc5c6ccccc6c(-c6ccccc6)cc5c34)cc2)cc1]

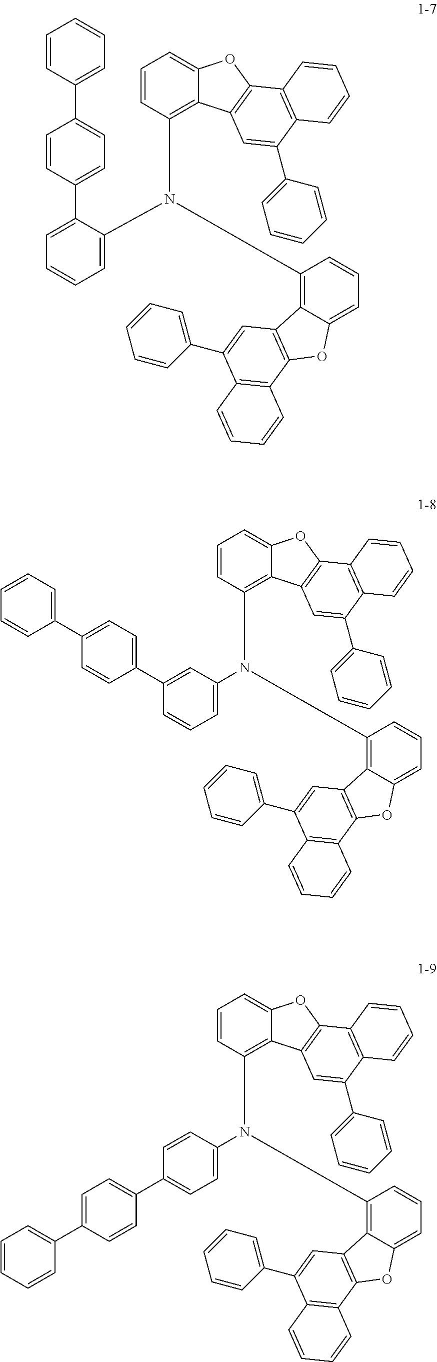 [SMILES: c1ccc(-c2ccc(-c3ccccc3N(c3cccc4oc5c6ccccc6c(-c6ccccc6)cc5c34)c3cccc4oc5c6ccccc6c(-c6ccccc6)cc5c34)cc2)cc1 ; c1ccc(-c2ccc(-c3cccc(N(c4cccc5oc6c7ccccc7c(-c7ccccc7)cc6c45)c4cccc5oc6c7ccccc7c(-c7ccccc7)cc6c45)c3)cc2)cc1 ; c1ccc(-c2ccc(-c3ccc(N(c4cccc5oc6c7ccccc7c(-c7ccccc7)cc6c45)c4cccc5oc6c7ccccc7c(-c7ccccc7)cc6c45)cc3)cc2)cc1]

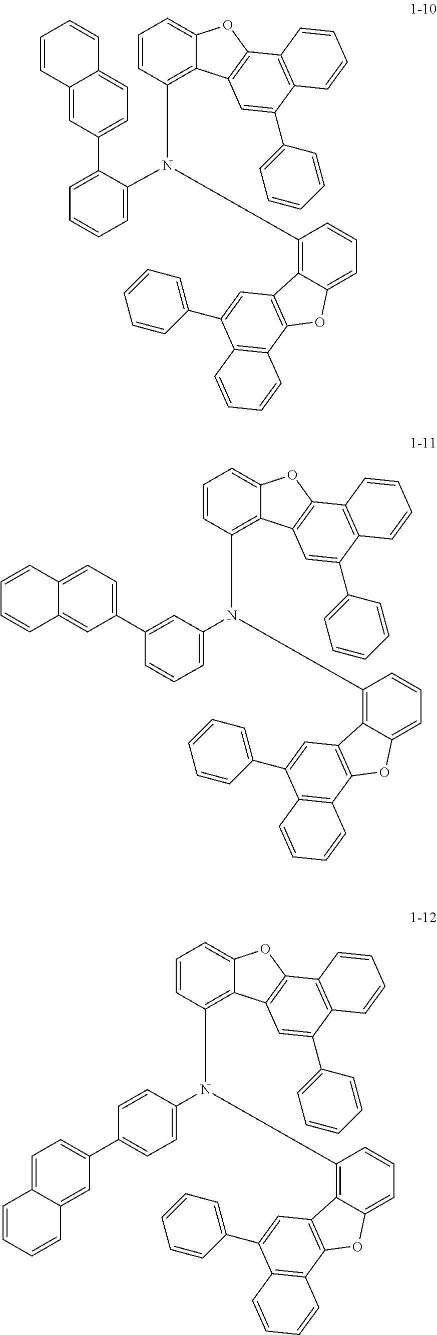 [SMILES: c1ccc(-c2cc3c(oc4cccc(N(c5ccccc5-c5ccc6ccccc6c5)c5cccc6oc7c8ccccc8c(-c8ccccc8)cc7c56)c43)c3ccccc23)cc1 ; c1ccc(-c2cc3c(oc4cccc(N(c5cccc(-c6ccc7ccccc7c6)c5)c5cccc6oc7c8ccccc8c(-c8ccccc8)cc7c56)c43)c3ccccc23)cc1 ; c1ccc(-c2cc3c(oc4cccc(N(c5ccc(-c6ccc7ccccc7c6)cc5)c5cccc6oc7c8ccccc8c(-c8ccccc8)cc7c56)c43)c3ccccc23)cc1]

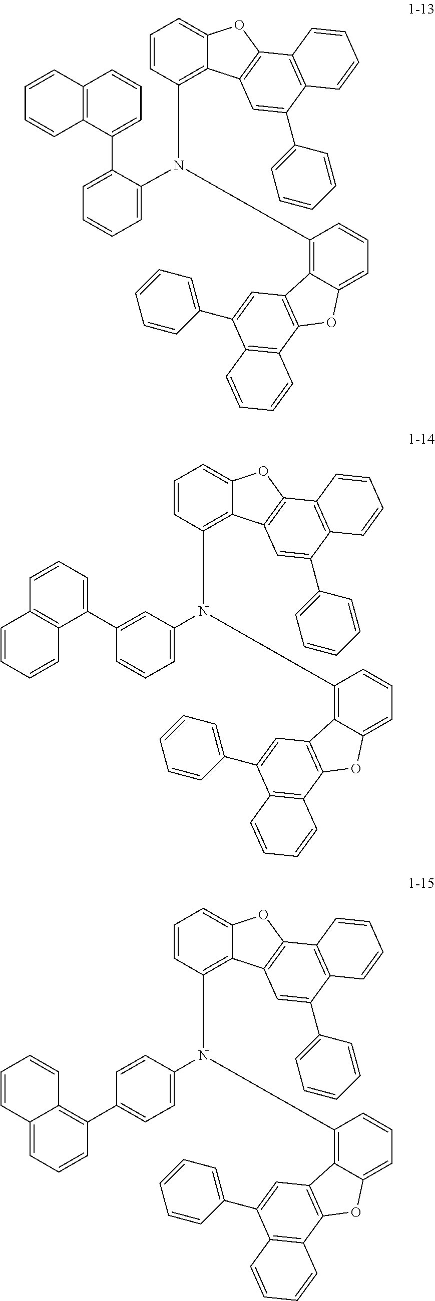 [SMILES: c1ccc(-c2cc3c(oc4cccc(N(c5ccccc5-c5cccc6ccccc56)c5cccc6oc7c8ccccc8c(-c8ccccc8)cc7c56)c43)c3ccccc23)cc1 ; c1ccc(-c2cc3c(oc4cccc(N(c5cccc(-c6cccc7ccccc67)c5)c5cccc6oc7c8ccccc8c(-c8ccccc8)cc7c56)c43)c3ccccc23)cc1 ; c1ccc(-c2cc3c(oc4cccc(N(c5ccc(-c6cccc7ccccc67)cc5)c5cccc6oc7c8ccccc8c(-c8ccccc8)cc7c56)c43)c3ccccc23)cc1]

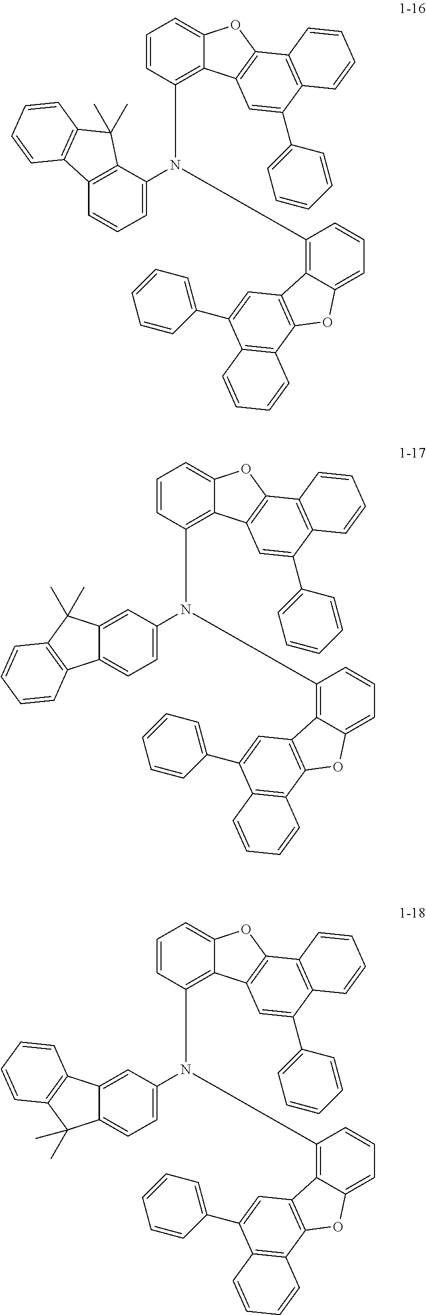 [SMILES: CC1(C)c2ccccc2-c2cccc(N(c3cccc4oc5c6ccccc6c(-c6ccccc6)cc5c34)c3cccc4oc5c6ccccc6c(-c6ccccc6)cc5c34)c21 ; CC1(C)c2ccccc2-c2ccc(N(c3cccc4oc5c6ccccc6c(-c6ccccc6)cc5c34)c3cccc4oc5c6ccccc6c(-c6ccccc6)cc5c34)cc21 ; CC1(C)c2ccccc2-c2cc(N(c3cccc4oc5c6ccccc6c(-c6ccccc6)cc5c34)c3cccc4oc5c6ccccc6c(-c6ccccc6)cc5c34)ccc21]

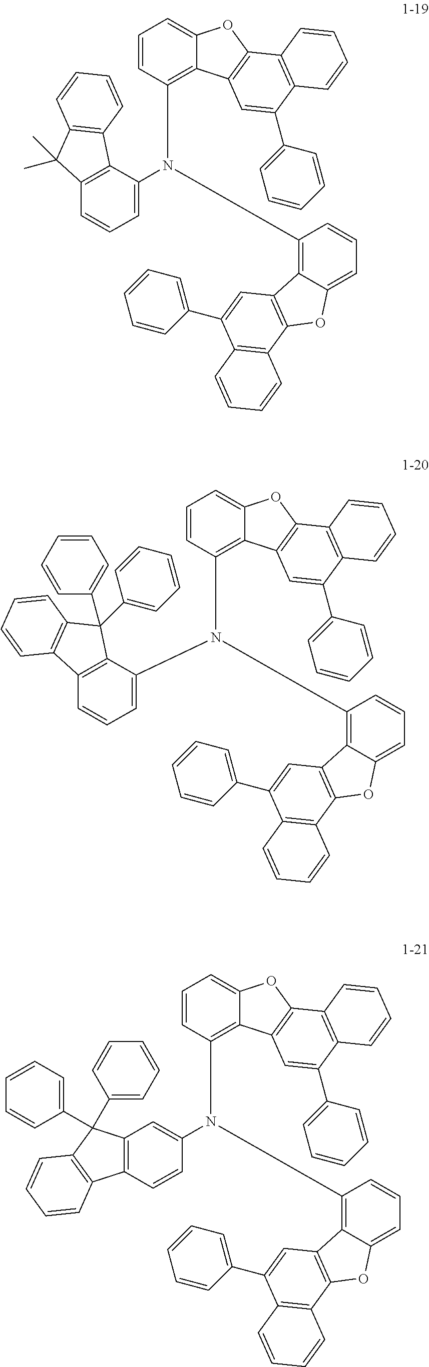 [SMILES: CC1(C)c2ccccc2-c2c(N(c3cccc4oc5c6ccccc6c(-c6ccccc6)cc5c34)c3cccc4oc5c6ccccc6c(-c6ccccc6)cc5c34)cccc21 ; c1ccc(-c2cc3c(oc4cccc(N(c5cccc6c5C(c5ccccc5)(c5ccccc5)c5ccccc5-6)c5cccc6oc7c8ccccc8c(-c8ccccc8)cc7c56)c43)c3ccccc23)cc1 ; c1ccc(-c2cc3c(oc4cccc(N(c5ccc6c(c5)C(c5ccccc5)(c5ccccc5)c5ccccc5-6)c5cccc6oc7c8ccccc8c(-c8ccccc8)cc7c56)c43)c3ccccc23)cc1]

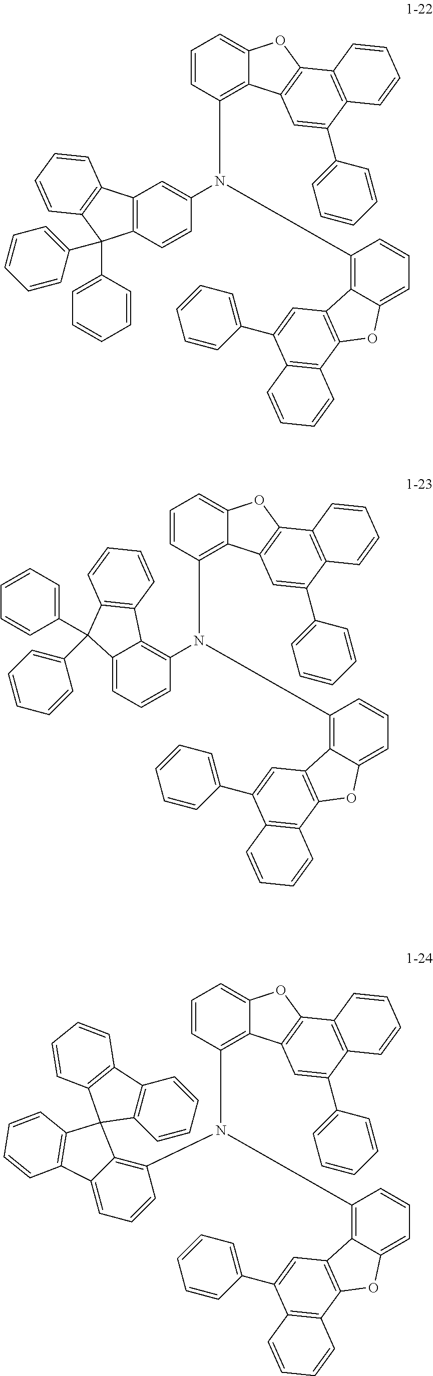 [SMILES: c1ccc(-c2cc3c(oc4cccc(N(c5ccc6c(c5)-c5ccccc5C6(c5ccccc5)c5ccccc5)c5cccc6oc7c8ccccc8c(-c8ccccc8)cc7c56)c43)c3ccccc23)cc1 ; c1ccc(-c2cc3c(oc4cccc(N(c5cccc6c5-c5ccccc5C6(c5ccccc5)c5ccccc5)c5cccc6oc7c8ccccc8c(-c8ccccc8)cc7c56)c43)c3ccccc23)cc1 ; c1ccc(-c2cc3c(oc4cccc(N(c5cccc6c5C5(c7ccccc7-c7ccccc75)c5ccccc5-6)c5cccc6oc7c8ccccc8c(-c8ccccc8)cc7c56)c43)c3ccccc23)cc1]

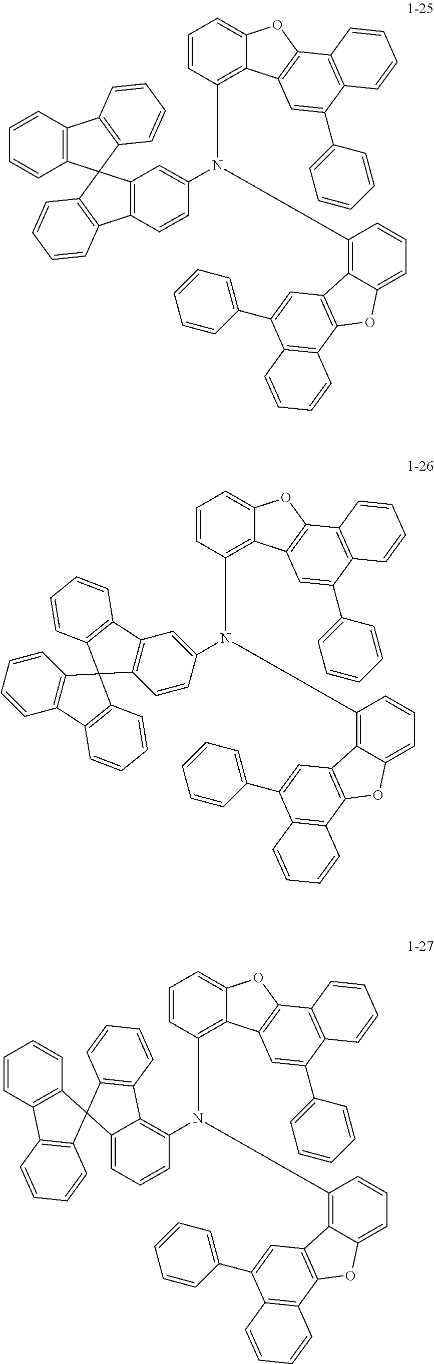 [SMILES: c1ccc(-c2cc3c(oc4cccc(N(c5ccc6c(c5)C5(c7ccccc7-c7ccccc75)c5ccccc5-6)c5cccc6oc7c8ccccc8c(-c8ccccc8)cc7c56)c43)c3ccccc23)cc1 ; c1ccc(-c2cc3c(oc4cccc(N(c5ccc6c(c5)-c5ccccc5C65c6ccccc6-c6ccccc65)c5cccc6oc7c8ccccc8c(-c8ccccc8)cc7c56)c43)c3ccccc23)cc1 ; c1ccc(-c2cc3c(oc4cccc(N(c5cccc6c5-c5ccccc5C65c6ccccc6-c6ccccc65)c5cccc6oc7c8ccccc8c(-c8ccccc8)cc7c56)c43)c3ccccc23)cc1]

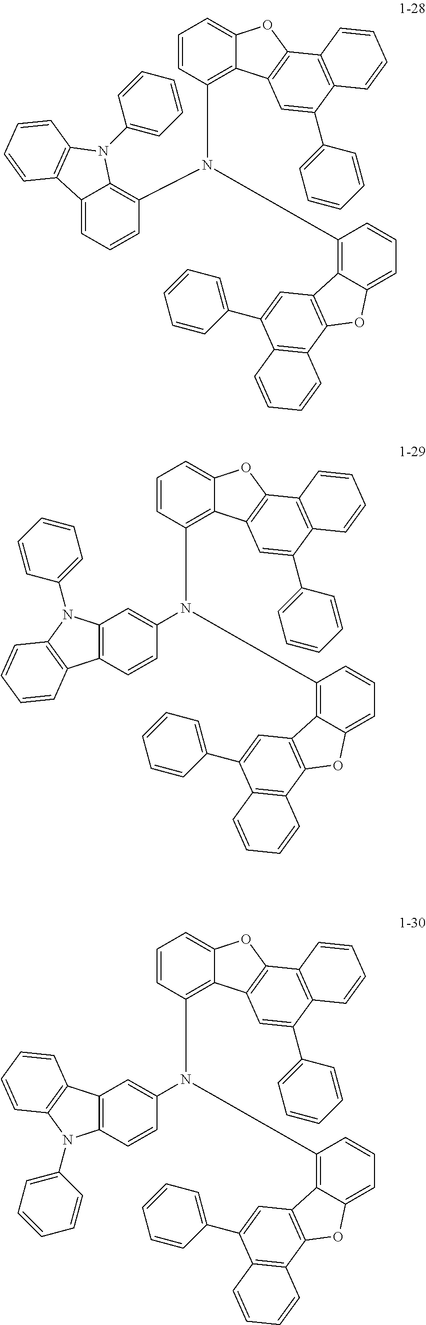 [SMILES: c1ccc(-c2cc3c(oc4cccc(N(c5cccc6oc7c8ccccc8c(-c8ccccc8)cc7c56)c5cccc6c7ccccc7n(-c7ccccc7)c56)c43)c3ccccc23)cc1 ; c1ccc(-c2cc3c(oc4cccc(N(c5ccc6c7ccccc7n(-c7ccccc7)c6c5)c5cccc6oc7c8ccccc8c(-c8ccccc8)cc7c56)c43)c3ccccc23)cc1 ; c1ccc(-c2cc3c(oc4cccc(N(c5ccc6c(c5)c5ccccc5n6-c5ccccc5)c5cccc6oc7c8ccccc8c(-c8ccccc8)cc7c56)c43)c3ccccc23)cc1]

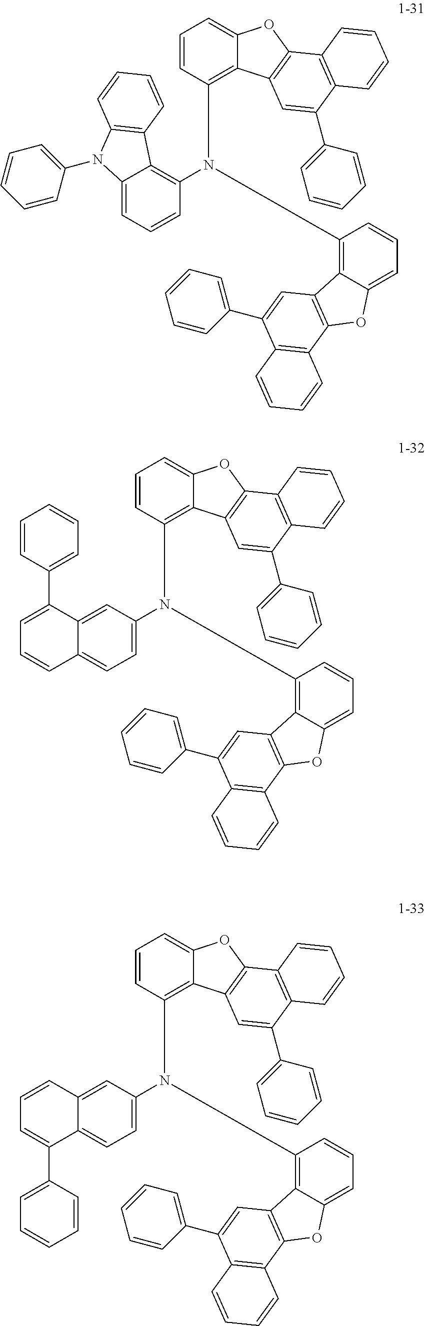 [SMILES: c1ccc(-c2cc3c(oc4cccc(N(c5cccc6oc7c8ccccc8c(-c8ccccc8)cc7c56)c5cccc6c5c5ccccc5n6-c5ccccc5)c43)c3ccccc23)cc1 ; c1ccc(-c2cccc3ccc(N(c4cccc5oc6c7ccccc7c(-c7ccccc7)cc6c45)c4cccc5oc6c7ccccc7c(-c7ccccc7)cc6c45)cc23)cc1 ; c1ccc(-c2cccc3cc(N(c4cccc5oc6c7ccccc7c(-c7ccccc7)cc6c45)c4cccc5oc6c7ccccc7c(-c7ccccc7)cc6c45)ccc23)cc1]

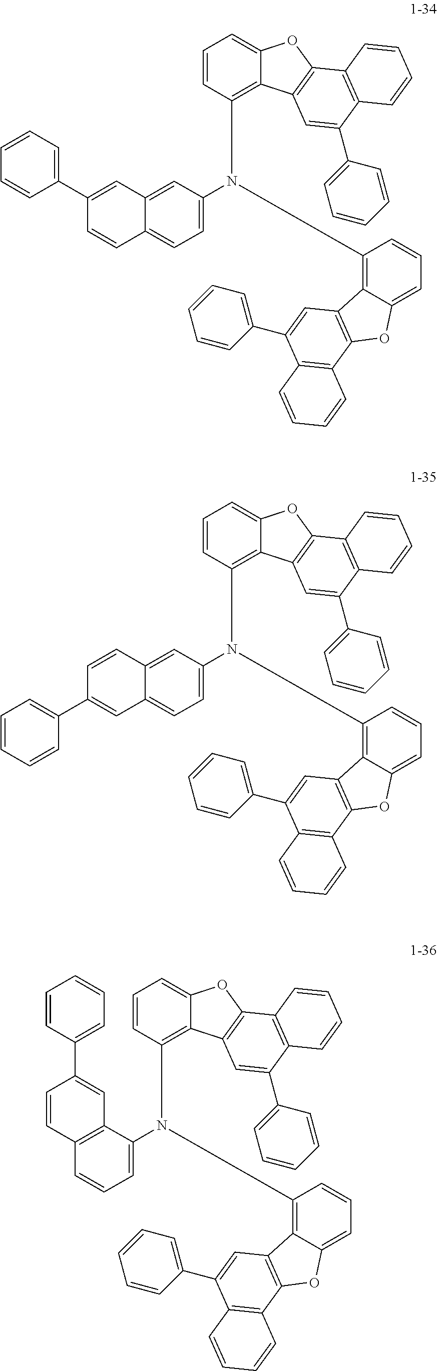 [SMILES: c1ccc(-c2ccc3ccc(N(c4cccc5oc6c7ccccc7c(-c7ccccc7)cc6c45)c4cccc5oc6c7ccccc7c(-c7ccccc7)cc6c45)cc3c2)cc1 ; c1ccc(-c2ccc3cc(N(c4cccc5oc6c7ccccc7c(-c7ccccc7)cc6c45)c4cccc5oc6c7ccccc7c(-c7ccccc7)cc6c45)ccc3c2)cc1 ; c1ccc(-c2ccc3cccc(N(c4cccc5oc6c7ccccc7c(-c7ccccc7)cc6c45)c4cccc5oc6c7ccccc7c(-c7ccccc7)cc6c45)c3c2)cc1]

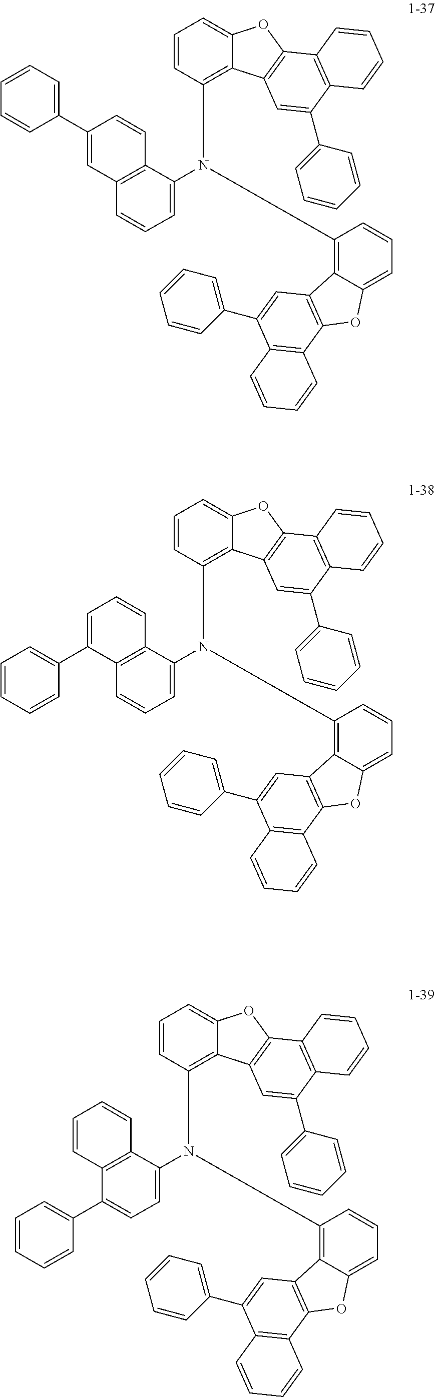 [SMILES: c1ccc(-c2ccc3c(N(c4cccc5oc6c7ccccc7c(-c7ccccc7)cc6c45)c4cccc5oc6c7ccccc7c(-c7ccccc7)cc6c45)cccc3c2)cc1 ; c1ccc(-c2cccc3c(N(c4cccc5oc6c7ccccc7c(-c7ccccc7)cc6c45)c4cccc5oc6c7ccccc7c(-c7ccccc7)cc6c45)cccc23)cc1 ; c1ccc(-c2ccc(N(c3cccc4oc5c6ccccc6c(-c6ccccc6)cc5c34)c3cccc4oc5c6ccccc6c(-c6ccccc6)cc5c34)c3ccccc23)cc1]

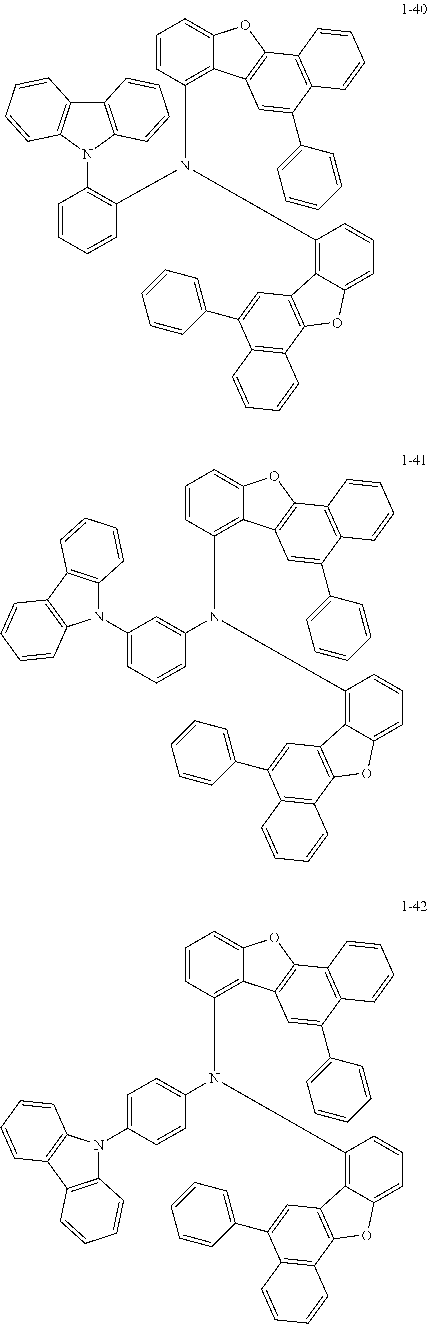 [SMILES: c1ccc(-c2cc3c(oc4cccc(N(c5ccccc5-n5c6ccccc6c6ccccc65)c5cccc6oc7c8ccccc8c(-c8ccccc8)cc7c56)c43)c3ccccc23)cc1 ; c1ccc(-c2cc3c(oc4cccc(N(c5cccc(-n6c7ccccc7c7ccccc76)c5)c5cccc6oc7c8ccccc8c(-c8ccccc8)cc7c56)c43)c3ccccc23)cc1 ; c1ccc(-c2cc3c(oc4cccc(N(c5ccc(-n6c7ccccc7c7ccccc76)cc5)c5cccc6oc7c8ccccc8c(-c8ccccc8)cc7c56)c43)c3ccccc23)cc1]

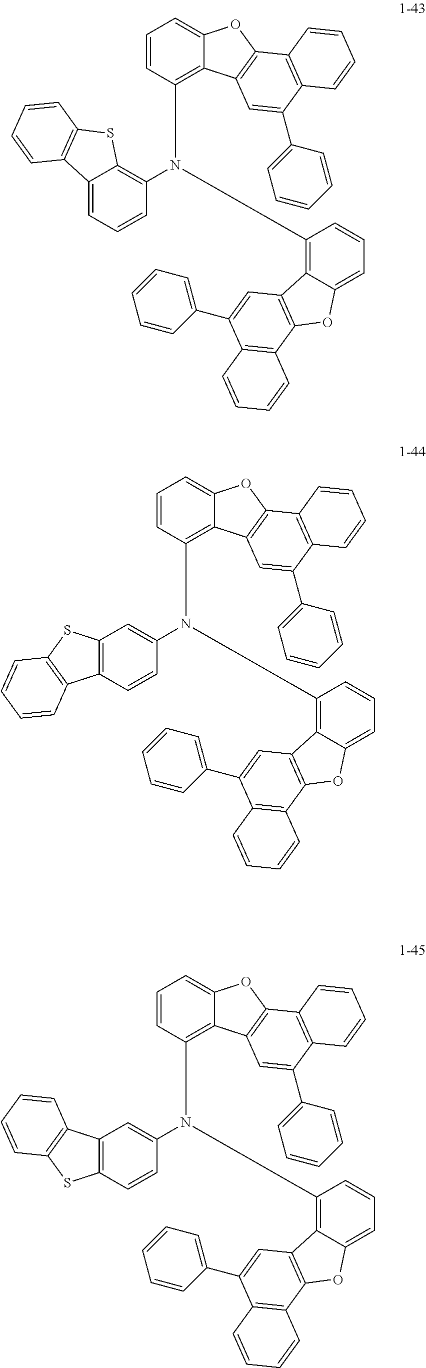 [SMILES: c1ccc(-c2cc3c(oc4cccc(N(c5cccc6c5sc5ccccc56)c5cccc6oc7c8ccccc8c(-c8ccccc8)cc7c56)c43)c3ccccc23)cc1 ; c1ccc(-c2cc3c(oc4cccc(N(c5ccc6c(c5)sc5ccccc56)c5cccc6oc7c8ccccc8c(-c8ccccc8)cc7c56)c43)c3ccccc23)cc1 ; c1ccc(-c2cc3c(oc4cccc(N(c5ccc6sc7ccccc7c6c5)c5cccc6oc7c8ccccc8c(-c8ccccc8)cc7c56)c43)c3ccccc23)cc1]

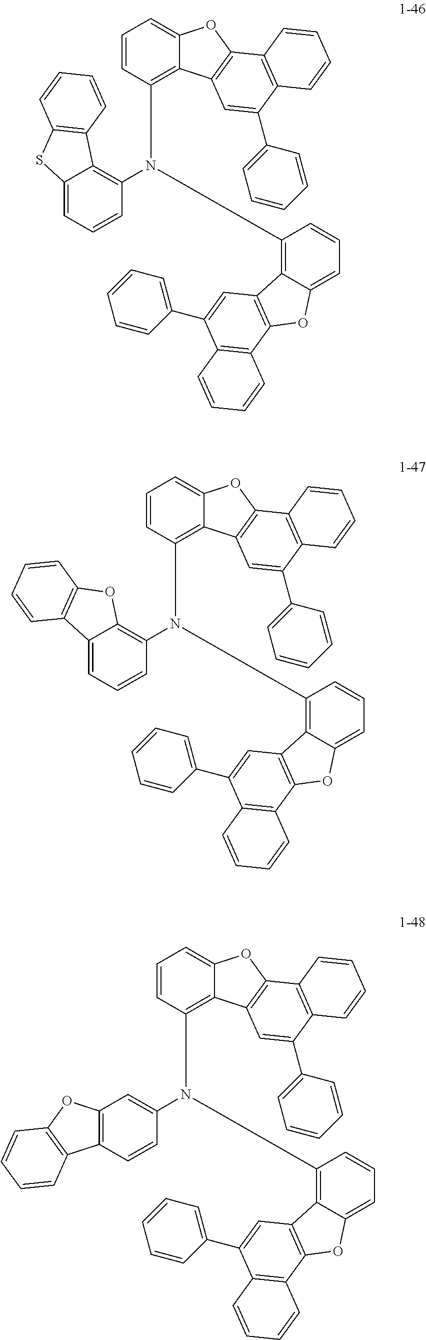 [SMILES: c1ccc(-c2cc3c(oc4cccc(N(c5cccc6oc7c8ccccc8c(-c8ccccc8)cc7c56)c5cccc6sc7ccccc7c56)c43)c3ccccc23)cc1 ; c1ccc(-c2cc3c(oc4cccc(N(c5cccc6c5oc5ccccc56)c5cccc6oc7c8ccccc8c(-c8ccccc8)cc7c56)c43)c3ccccc23)cc1 ; c1ccc(-c2cc3c(oc4cccc(N(c5ccc6c(c5)oc5ccccc56)c5cccc6oc7c8ccccc8c(-c8ccccc8)cc7c56)c43)c3ccccc23)cc1]

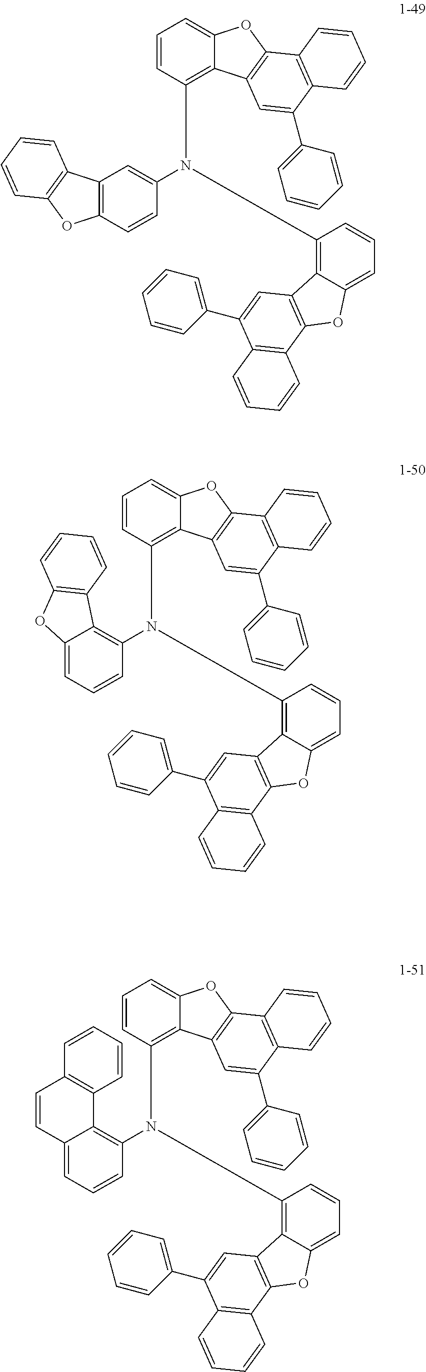 [SMILES: c1ccc(-c2cc3c(oc4cccc(N(c5ccc6oc7ccccc7c6c5)c5cccc6oc7c8ccccc8c(-c8ccccc8)cc7c56)c43)c3ccccc23)cc1 ; c1ccc(-c2cc3c(oc4cccc(N(c5cccc6oc7ccccc7c56)c5cccc6oc7c8ccccc8c(-c8ccccc8)cc7c56)c43)c3ccccc23)cc1 ; c1ccc(-c2cc3c(oc4cccc(N(c5cccc6ccc7ccccc7c56)c5cccc6oc7c8ccccc8c(-c8ccccc8)cc7c56)c43)c3ccccc23)cc1]

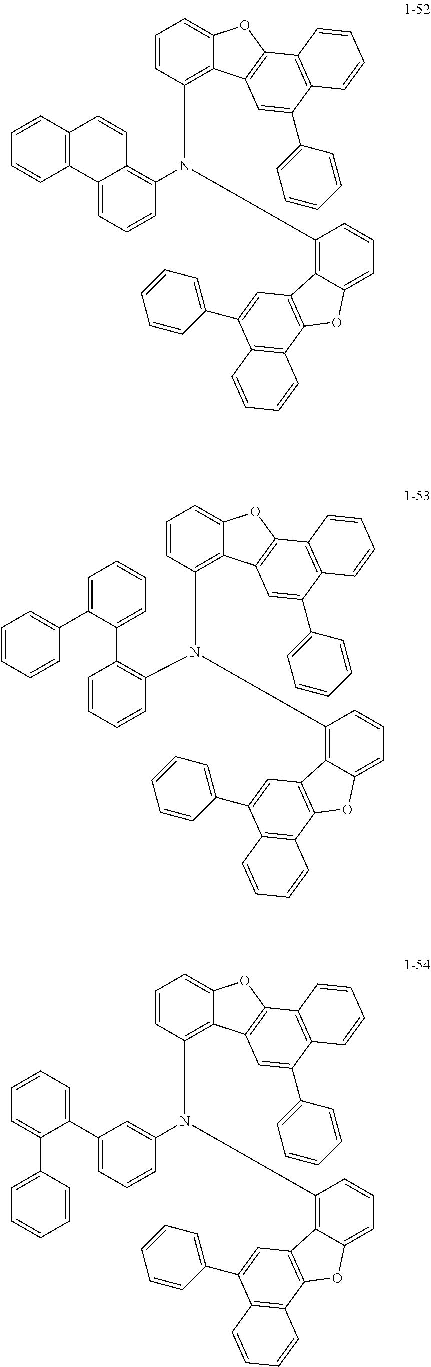 [SMILES: c1ccc(-c2cc3c(oc4cccc(N(c5cccc6c5ccc5ccccc56)c5cccc6oc7c8ccccc8c(-c8ccccc8)cc7c56)c43)c3ccccc23)cc1 ; c1ccc(-c2ccccc2-c2ccccc2N(c2cccc3oc4c5ccccc5c(-c5ccccc5)cc4c23)c2cccc3oc4c5ccccc5c(-c5ccccc5)cc4c23)cc1 ; c1ccc(-c2ccccc2-c2cccc(N(c3cccc4oc5c6ccccc6c(-c6ccccc6)cc5c34)c3cccc4oc5c6ccccc6c(-c6ccccc6)cc5c34)c2)cc1]

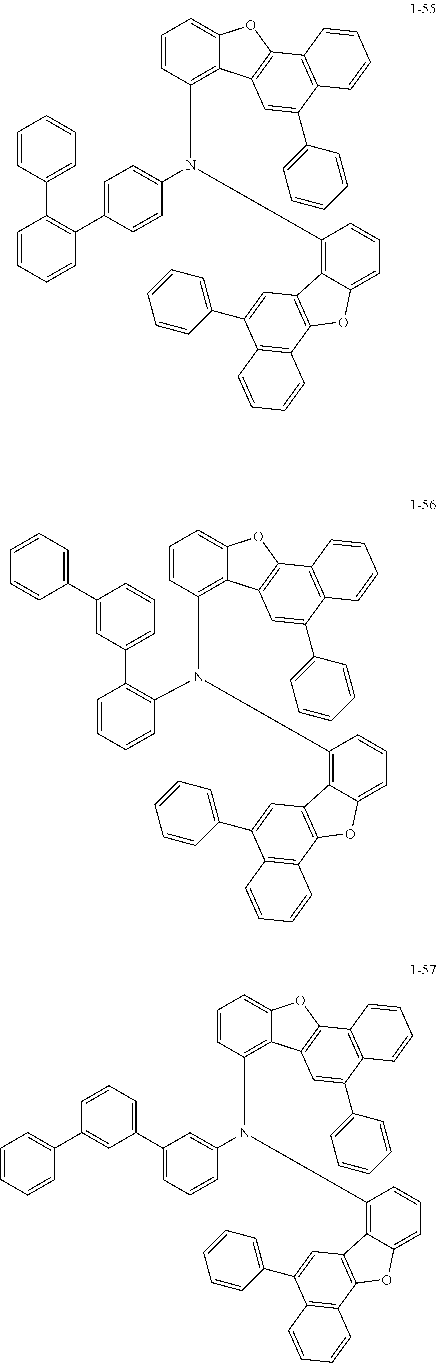 [SMILES: c1ccc(-c2ccccc2-c2ccc(N(c3cccc4oc5c6ccccc6c(-c6ccccc6)cc5c34)c3cccc4oc5c6ccccc6c(-c6ccccc6)cc5c34)cc2)cc1 ; c1ccc(-c2cccc(-c3ccccc3N(c3cccc4oc5c6ccccc6c(-c6ccccc6)cc5c34)c3cccc4oc5c6ccccc6c(-c6ccccc6)cc5c34)c2)cc1 ; c1ccc(-c2cccc(-c3cccc(N(c4cccc5oc6c7ccccc7c(-c7ccccc7)cc6c45)c4cccc5oc6c7ccccc7c(-c7ccccc7)cc6c45)c3)c2)cc1]

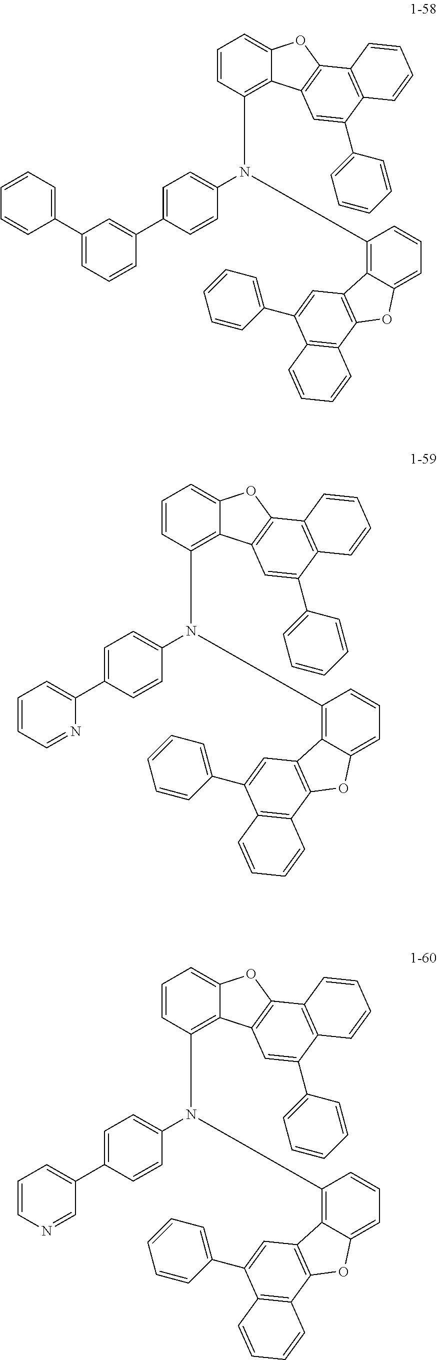 [SMILES: c1ccc(-c2cccc(-c3ccc(N(c4cccc5oc6c7ccccc7c(-c7ccccc7)cc6c45)c4cccc5oc6c7ccccc7c(-c7ccccc7)cc6c45)cc3)c2)cc1 ; c1ccc(-c2cc3c(oc4cccc(N(c5ccc(-c6ccccn6)cc5)c5cccc6oc7c8ccccc8c(-c8ccccc8)cc7c56)c43)c3ccccc23)cc1 ; c1ccc(-c2cc3c(oc4cccc(N(c5ccc(-c6cccnc6)cc5)c5cccc6oc7c8ccccc8c(-c8ccccc8)cc7c56)c43)c3ccccc23)cc1]

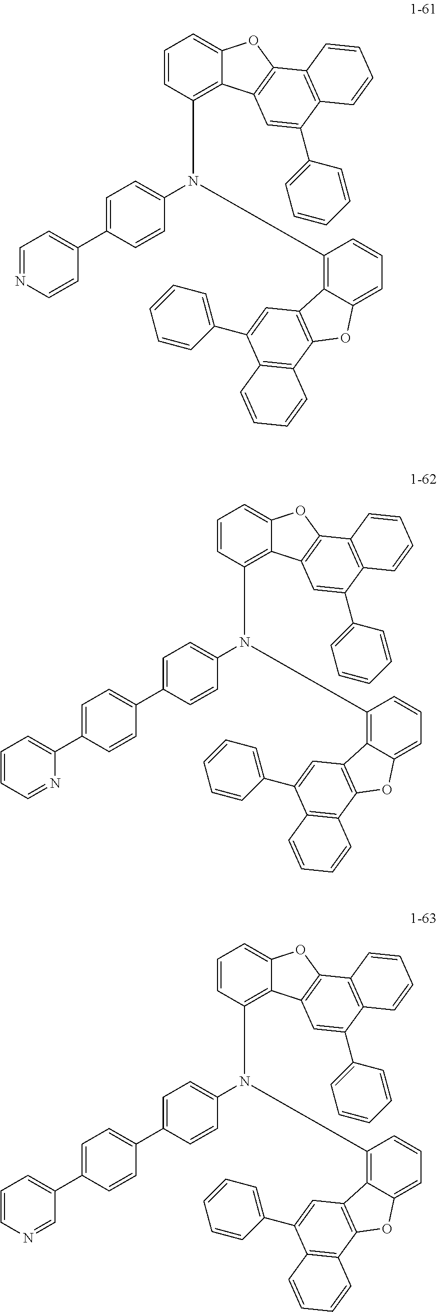 [SMILES: c1ccc(-c2cc3c(oc4cccc(N(c5ccc(-c6ccncc6)cc5)c5cccc6oc7c8ccccc8c(-c8ccccc8)cc7c56)c43)c3ccccc23)cc1 ; c1ccc(-c2cc3c(oc4cccc(N(c5ccc(-c6ccc(-c7ccccn7)cc6)cc5)c5cccc6oc7c8ccccc8c(-c8ccccc8)cc7c56)c43)c3ccccc23)cc1 ; c1ccc(-c2cc3c(oc4cccc(N(c5ccc(-c6ccc(-c7cccnc7)cc6)cc5)c5cccc6oc7c8ccccc8c(-c8ccccc8)cc7c56)c43)c3ccccc23)cc1]

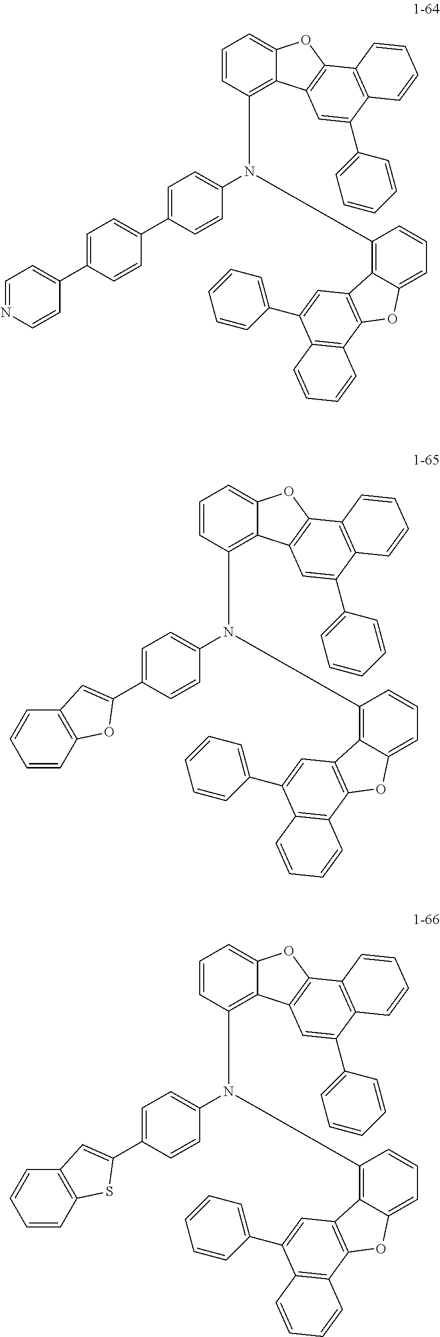 [SMILES: c1ccc(-c2cc3c(oc4cccc(N(c5ccc(-c6ccc(-c7ccncc7)cc6)cc5)c5cccc6oc7c8ccccc8c(-c8ccccc8)cc7c56)c43)c3ccccc23)cc1 ; c1ccc(-c2cc3c(oc4cccc(N(c5ccc(-c6cc7ccccc7o6)cc5)c5cccc6oc7c8ccccc8c(-c8ccccc8)cc7c56)c43)c3ccccc23)cc1 ; c1ccc(-c2cc3c(oc4cccc(N(c5ccc(-c6cc7ccccc7s6)cc5)c5cccc6oc7c8ccccc8c(-c8ccccc8)cc7c56)c43)c3ccccc23)cc1]

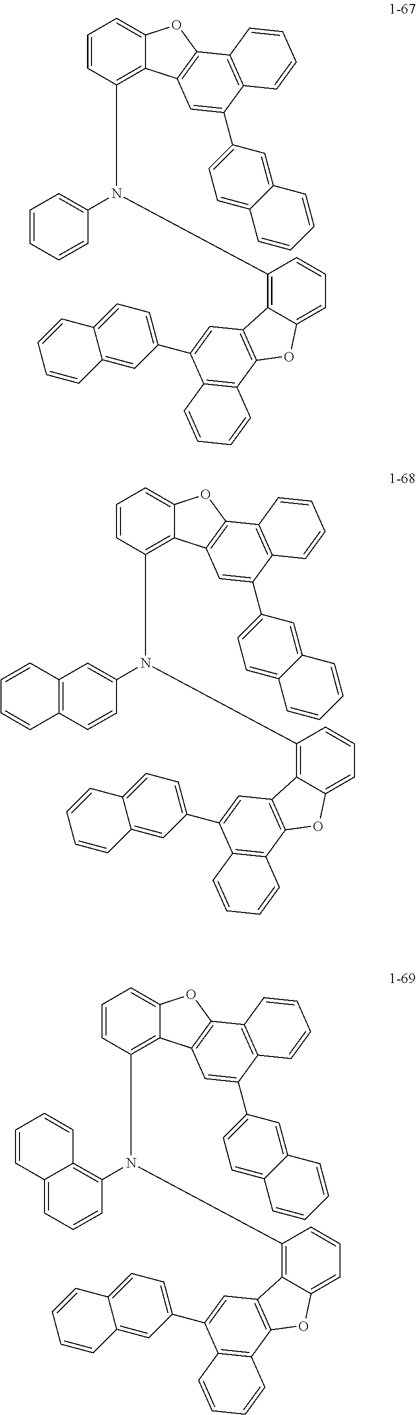 [SMILES: c1ccc(N(c2cccc3oc4c5ccccc5c(-c5ccc6ccccc6c5)cc4c23)c2cccc3oc4c5ccccc5c(-c5ccc6ccccc6c5)cc4c23)cc1 ; c1ccc2cc(-c3cc4c(oc5cccc(N(c6ccc7ccccc7c6)c6cccc7oc8c9ccccc9c(-c9ccc%10ccccc%10c9)cc8c67)c54)c4ccccc34)ccc2c1 ; c1ccc2cc(-c3cc4c(oc5cccc(N(c6cccc7ccccc67)c6cccc7oc8c9ccccc9c(-c9ccc%10ccccc%10c9)cc8c67)c54)c4ccccc34)ccc2c1]

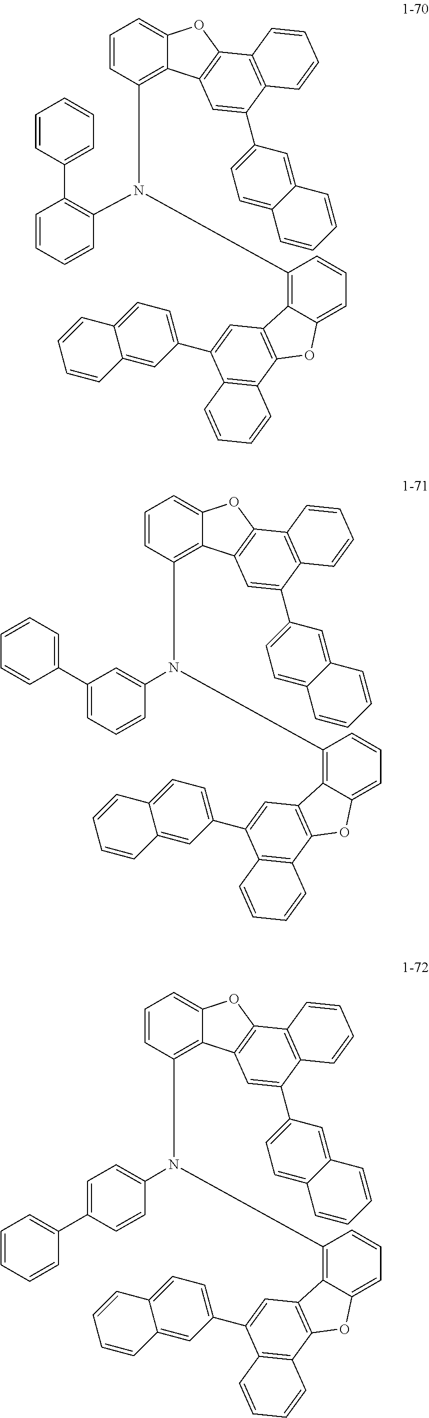 [SMILES: c1ccc(-c2ccccc2N(c2cccc3oc4c5ccccc5c(-c5ccc6ccccc6c5)cc4c23)c2cccc3oc4c5ccccc5c(-c5ccc6ccccc6c5)cc4c23)cc1 ; c1ccc(-c2cccc(N(c3cccc4oc5c6ccccc6c(-c6ccc7ccccc7c6)cc5c34)c3cccc4oc5c6ccccc6c(-c6ccc7ccccc7c6)cc5c34)c2)cc1 ; c1ccc(-c2ccc(N(c3cccc4oc5c6ccccc6c(-c6ccc7ccccc7c6)cc5c34)c3cccc4oc5c6ccccc6c(-c6ccc7ccccc7c6)cc5c34)cc2)cc1]

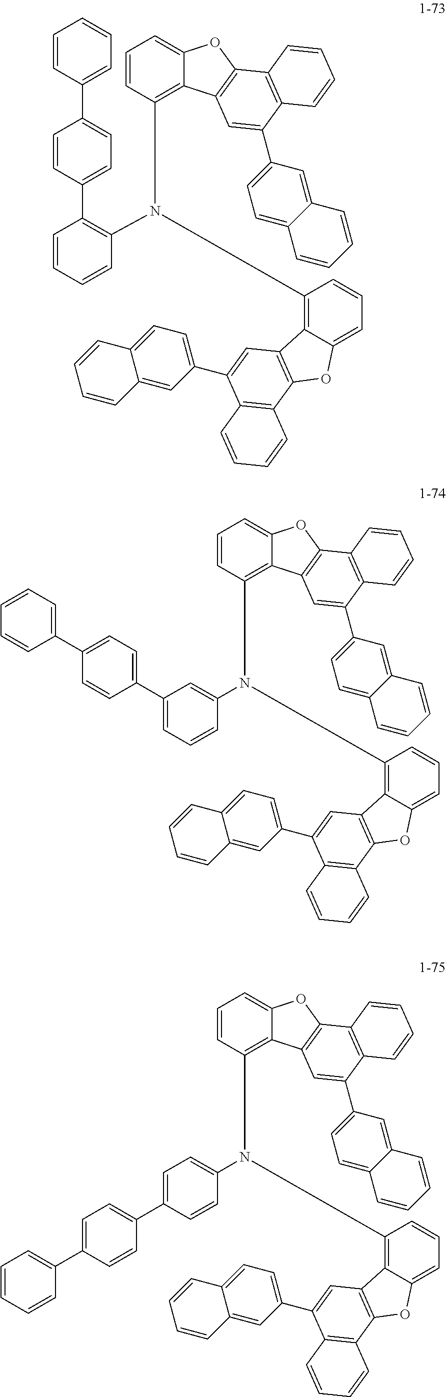 [SMILES: c1ccc(-c2ccc(-c3ccccc3N(c3cccc4oc5c6ccccc6c(-c6ccc7ccccc7c6)cc5c34)c3cccc4oc5c6ccccc6c(-c6ccc7ccccc7c6)cc5c34)cc2)cc1 ; c1ccc(-c2ccc(-c3cccc(N(c4cccc5oc6c7ccccc7c(-c7ccc8ccccc8c7)cc6c45)c4cccc5oc6c7ccccc7c(-c7ccc8ccccc8c7)cc6c45)c3)cc2)cc1 ; c1ccc(-c2ccc(-c3ccc(N(c4cccc5oc6c7ccccc7c(-c7ccc8ccccc8c7)cc6c45)c4cccc5oc6c7ccccc7c(-c7ccc8ccccc8c7)cc6c45)cc3)cc2)cc1]

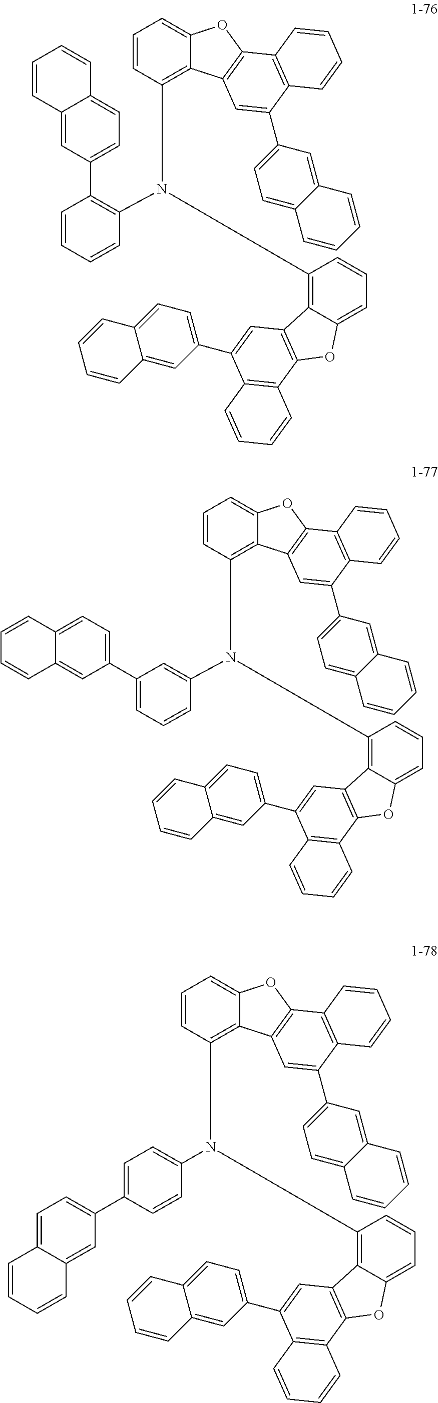 [SMILES: c1ccc(N(c2cccc3oc4c5ccccc5c(-c5ccc6ccccc6c5)cc4c23)c2cccc3oc4c5ccccc5c(-c5ccc6ccccc6c5)cc4c23)c(-c2ccc3ccccc3c2)c1 ; c1cc(-c2ccc3ccccc3c2)cc(N(c2cccc3oc4c5ccccc5c(-c5ccc6ccccc6c5)cc4c23)c2cccc3oc4c5ccccc5c(-c5ccc6ccccc6c5)cc4c23)c1 ; c1ccc2cc(-c3ccc(N(c4cccc5oc6c7ccccc7c(-c7ccc8ccccc8c7)cc6c45)c4cccc5oc6c7ccccc7c(-c7ccc8ccccc8c7)cc6c45)cc3)ccc2c1]

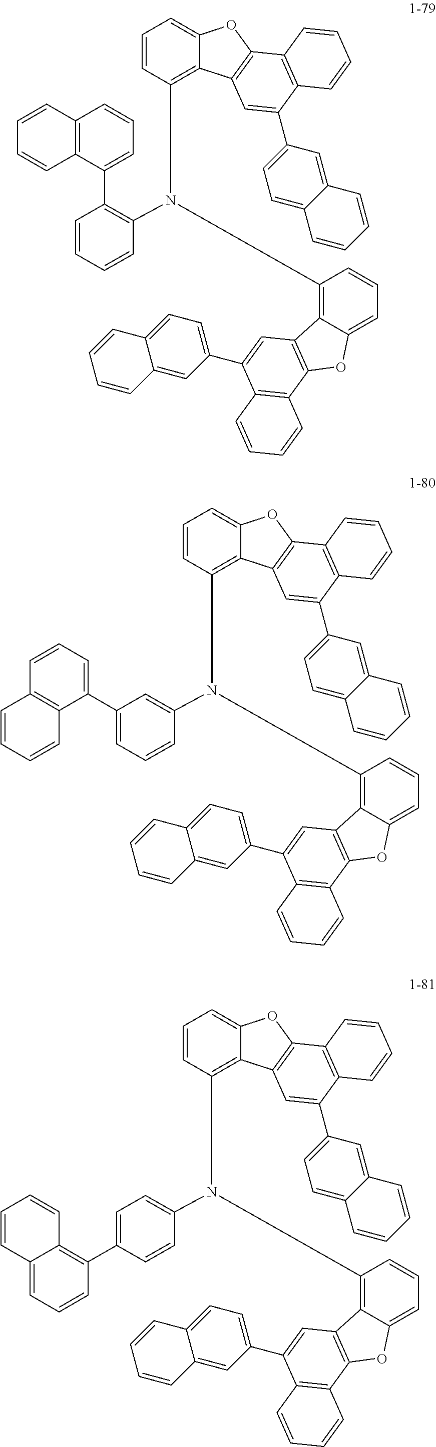 [SMILES: c1ccc(N(c2cccc3oc4c5ccccc5c(-c5ccc6ccccc6c5)cc4c23)c2cccc3oc4c5ccccc5c(-c5ccc6ccccc6c5)cc4c23)c(-c2cccc3ccccc23)c1 ; c1cc(-c2cccc3ccccc23)cc(N(c2cccc3oc4c5ccccc5c(-c5ccc6ccccc6c5)cc4c23)c2cccc3oc4c5ccccc5c(-c5ccc6ccccc6c5)cc4c23)c1 ; c1ccc2cc(-c3cc4c(oc5cccc(N(c6ccc(-c7cccc8ccccc78)cc6)c6cccc7oc8c9ccccc9c(-c9ccc%10ccccc%10c9)cc8c67)c54)c4ccccc34)ccc2c1]

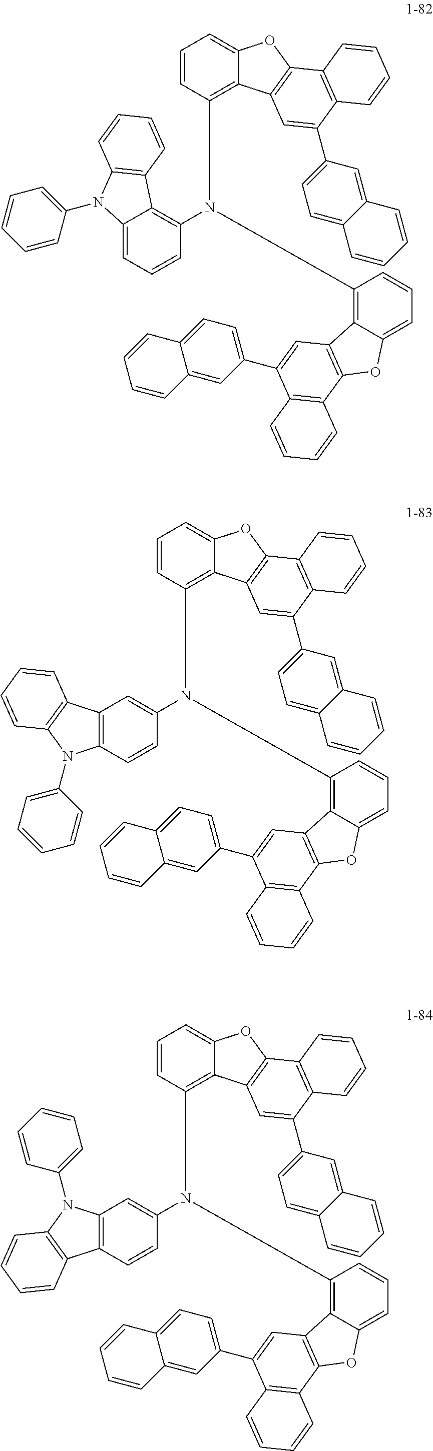 [SMILES: c1ccc(-n2c3ccccc3c3c(N(c4cccc5oc6c7ccccc7c(-c7ccc8ccccc8c7)cc6c45)c4cccc5oc6c7ccccc7c(-c7ccc8ccccc8c7)cc6c45)cccc32)cc1 ; c1ccc(-n2c3ccccc3c3cc(N(c4cccc5oc6c7ccccc7c(-c7ccc8ccccc8c7)cc6c45)c4cccc5oc6c7ccccc7c(-c7ccc8ccccc8c7)cc6c45)ccc32)cc1 ; c1ccc(-n2c3ccccc3c3ccc(N(c4cccc5oc6c7ccccc7c(-c7ccc8ccccc8c7)cc6c45)c4cccc5oc6c7ccccc7c(-c7ccc8ccccc8c7)cc6c45)cc32)cc1]

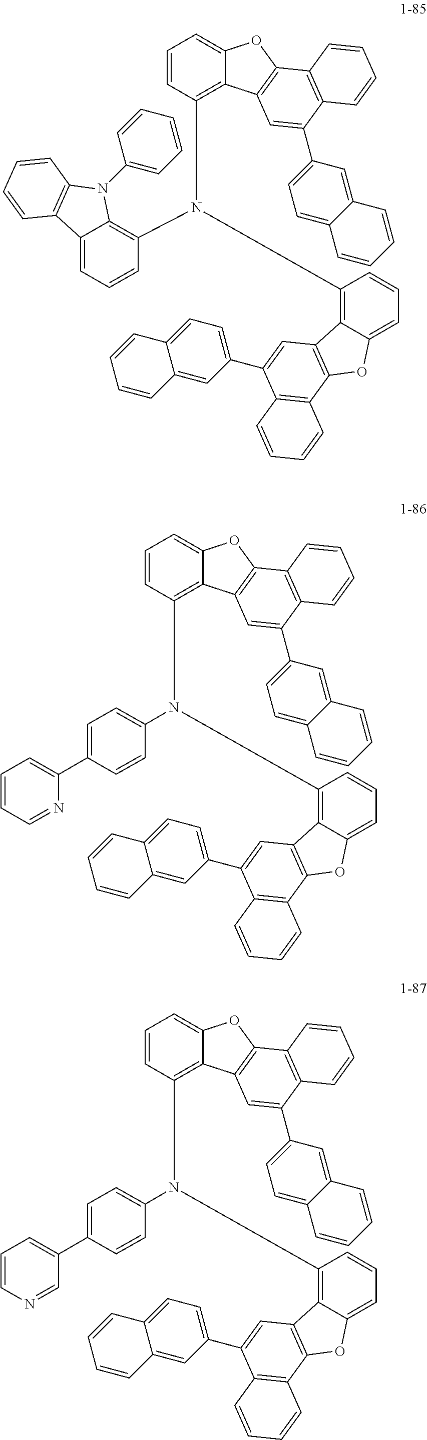 [SMILES: c1ccc(-n2c3ccccc3c3cccc(N(c4cccc5oc6c7ccccc7c(-c7ccc8ccccc8c7)cc6c45)c4cccc5oc6c7ccccc7c(-c7ccc8ccccc8c7)cc6c45)c32)cc1 ; c1ccc(-c2ccc(N(c3cccc4oc5c6ccccc6c(-c6ccc7ccccc7c6)cc5c34)c3cccc4oc5c6ccccc6c(-c6ccc7ccccc7c6)cc5c34)cc2)nc1 ; c1cncc(-c2ccc(N(c3cccc4oc5c6ccccc6c(-c6ccc7ccccc7c6)cc5c34)c3cccc4oc5c6ccccc6c(-c6ccc7ccccc7c6)cc5c34)cc2)c1]

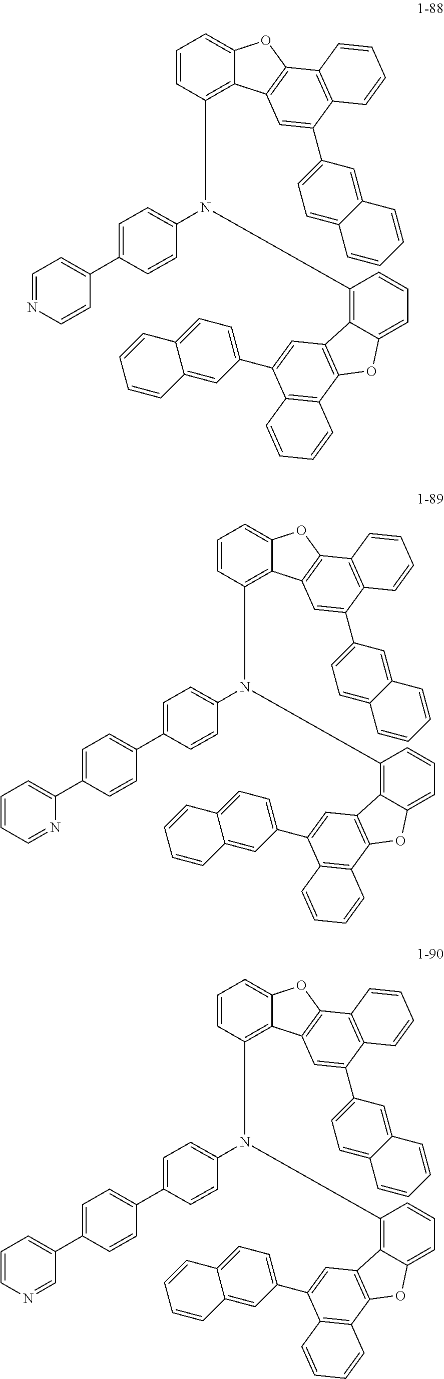 [SMILES: c1ccc2cc(-c3cc4c(oc5cccc(N(c6ccc(-c7ccncc7)cc6)c6cccc7oc8c9ccccc9c(-c9ccc%10ccccc%10c9)cc8c67)c54)c4ccccc34)ccc2c1 ; c1ccc(-c2ccc(-c3ccc(N(c4cccc5oc6c7ccccc7c(-c7ccc8ccccc8c7)cc6c45)c4cccc5oc6c7ccccc7c(-c7ccc8ccccc8c7)cc6c45)cc3)cc2)nc1 ; c1cncc(-c2ccc(-c3ccc(N(c4cccc5oc6c7ccccc7c(-c7ccc8ccccc8c7)cc6c45)c4cccc5oc6c7ccccc7c(-c7ccc8ccccc8c7)cc6c45)cc3)cc2)c1]

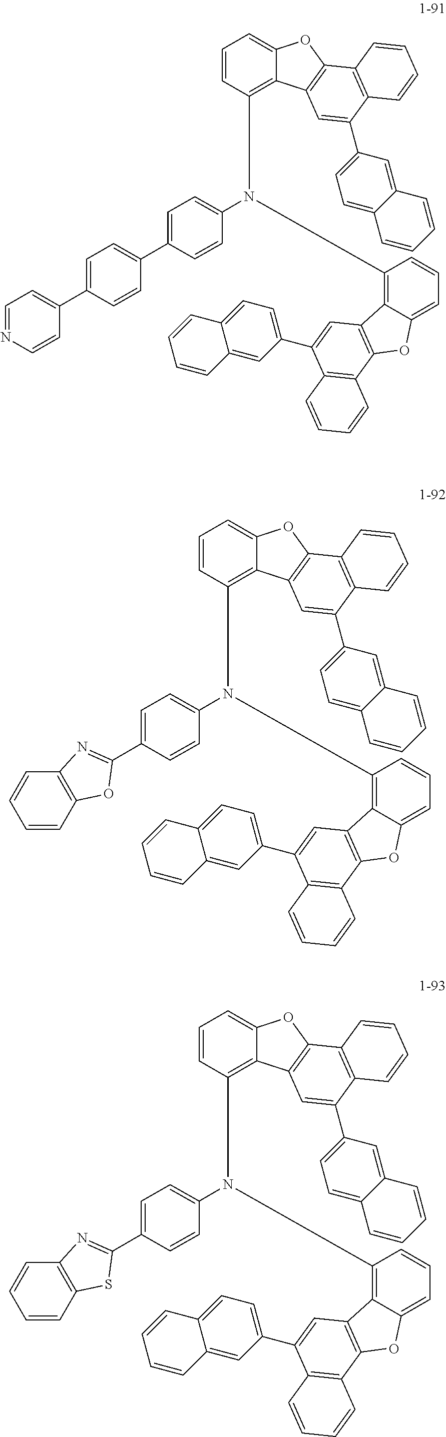 [SMILES: c1ccc2cc(-c3cc4c(oc5cccc(N(c6ccc(-c7ccc(-c8ccncc8)cc7)cc6)c6cccc7oc8c9ccccc9c(-c9ccc%10ccccc%10c9)cc8c67)c54)c4ccccc34)ccc2c1 ; c1ccc2cc(-c3cc4c(oc5cccc(N(c6ccc(-c7nc8ccccc8o7)cc6)c6cccc7oc8c9ccccc9c(-c9ccc%10ccccc%10c9)cc8c67)c54)c4ccccc34)ccc2c1 ; c1ccc2cc(-c3cc4c(oc5cccc(N(c6ccc(-c7nc8ccccc8s7)cc6)c6cccc7oc8c9ccccc9c(-c9ccc%10ccccc%10c9)cc8c67)c54)c4ccccc34)ccc2c1]

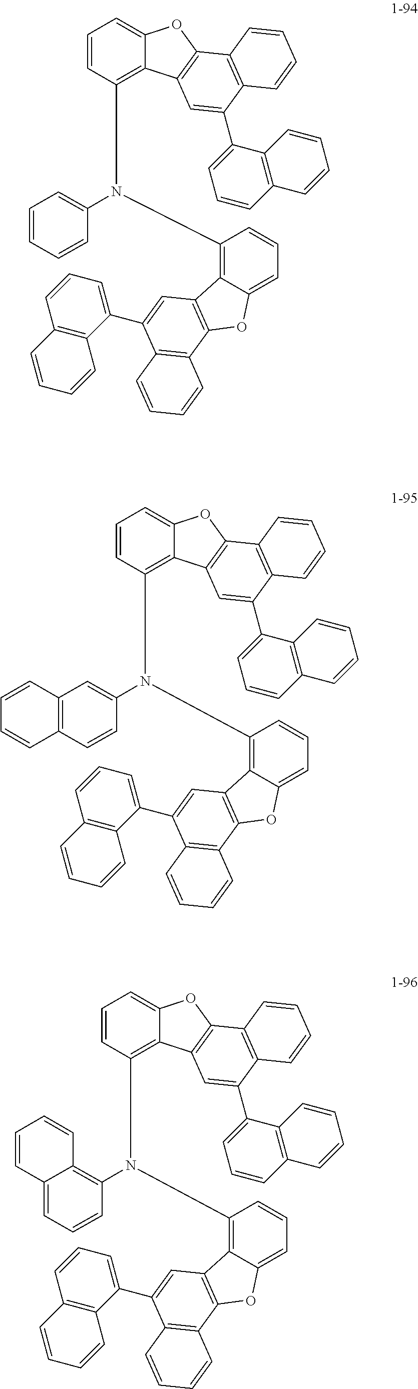 [SMILES: c1ccc(N(c2cccc3oc4c5ccccc5c(-c5cccc6ccccc56)cc4c23)c2cccc3oc4c5ccccc5c(-c5cccc6ccccc56)cc4c23)cc1 ; c1ccc2cc(N(c3cccc4oc5c6ccccc6c(-c6cccc7ccccc67)cc5c34)c3cccc4oc5c6ccccc6c(-c6cccc7ccccc67)cc5c34)ccc2c1 ; c1ccc2c(-c3cc4c(oc5cccc(N(c6cccc7ccccc67)c6cccc7oc8c9ccccc9c(-c9cccc%10ccccc9%10)cc8c67)c54)c4ccccc34)cccc2c1]

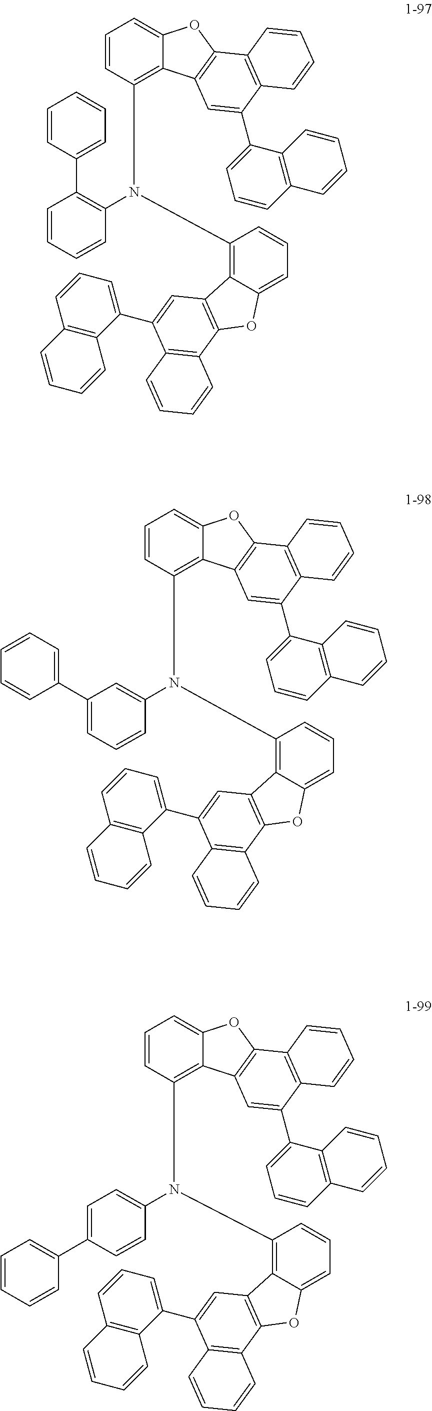 [SMILES: c1ccc(-c2ccccc2N(c2cccc3oc4c5ccccc5c(-c5cccc6ccccc56)cc4c23)c2cccc3oc4c5ccccc5c(-c5cccc6ccccc56)cc4c23)cc1 ; c1ccc(-c2cccc(N(c3cccc4oc5c6ccccc6c(-c6cccc7ccccc67)cc5c34)c3cccc4oc5c6ccccc6c(-c6cccc7ccccc67)cc5c34)c2)cc1 ; c1ccc(-c2ccc(N(c3cccc4oc5c6ccccc6c(-c6cccc7ccccc67)cc5c34)c3cccc4oc5c6ccccc6c(-c6cccc7ccccc67)cc5c34)cc2)cc1]

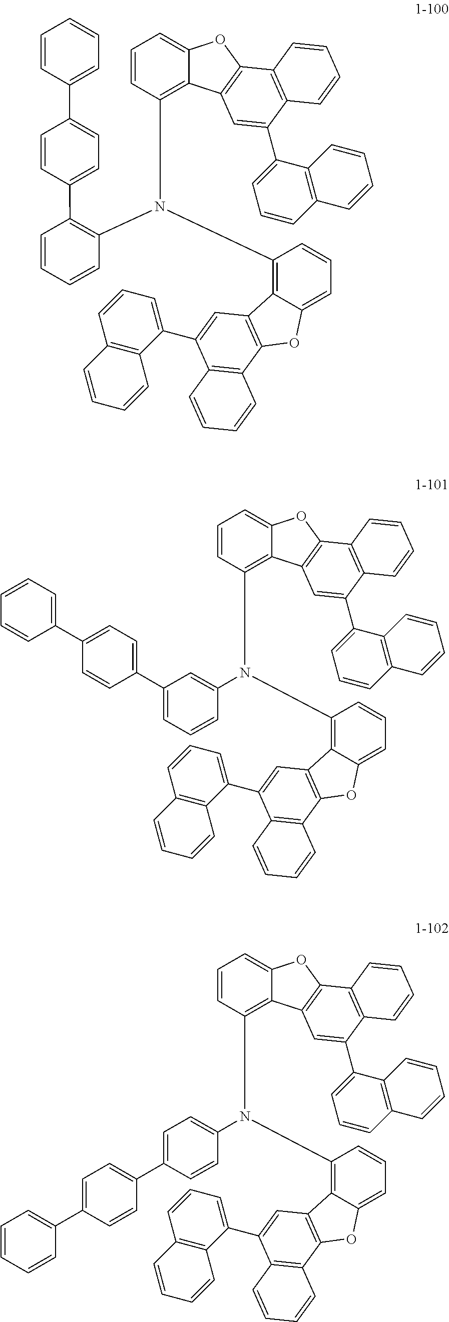 [SMILES: c1ccc(-c2ccc(-c3ccccc3N(c3cccc4oc5c6ccccc6c(-c6cccc7ccccc67)cc5c34)c3cccc4oc5c6ccccc6c(-c6cccc7ccccc67)cc5c34)cc2)cc1 ; c1ccc(-c2ccc(-c3cccc(N(c4cccc5oc6c7ccccc7c(-c7cccc8ccccc78)cc6c45)c4cccc5oc6c7ccccc7c(-c7cccc8ccccc78)cc6c45)c3)cc2)cc1 ; c1ccc(-c2ccc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2)cc1]

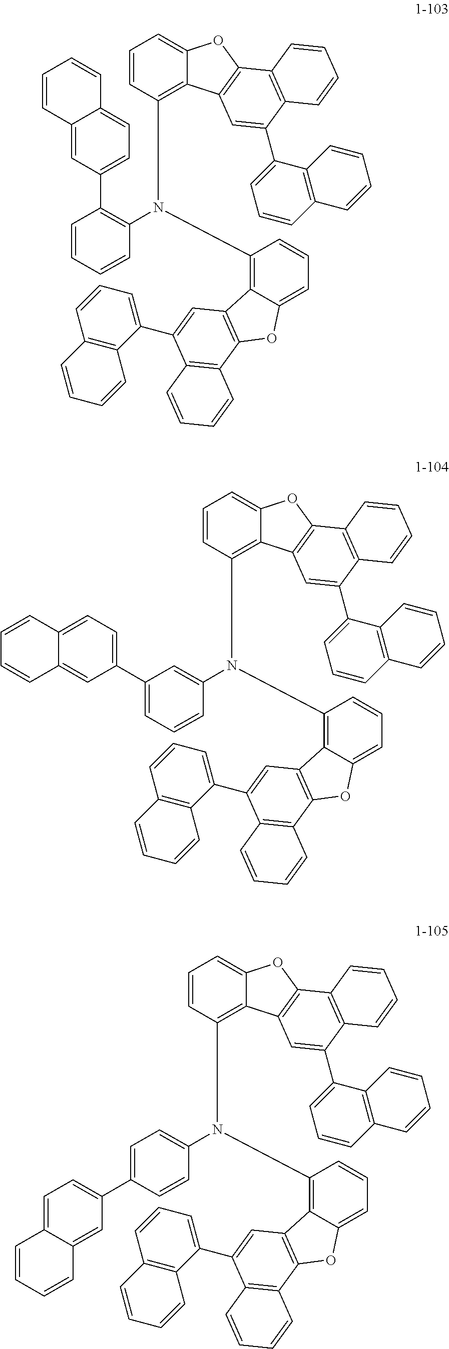 [SMILES: c1ccc(N(c2cccc3oc4c5ccccc5c(-c5cccc6ccccc56)cc4c23)c2cccc3oc4c5ccccc5c(-c5cccc6ccccc56)cc4c23)c(-c2ccc3ccccc3c2)c1 ; c1cc(-c2ccc3ccccc3c2)cc(N(c2cccc3oc4c5ccccc5c(-c5cccc6ccccc56)cc4c23)c2cccc3oc4c5ccccc5c(-c5cccc6ccccc56)cc4c23)c1 ; c1ccc2cc(-c3ccc(N(c4cccc5oc6c7ccccc7c(-c7cccc8ccccc78)cc6c45)c4cccc5oc6c7ccccc7c(-c7cccc8ccccc78)cc6c45)cc3)ccc2c1]

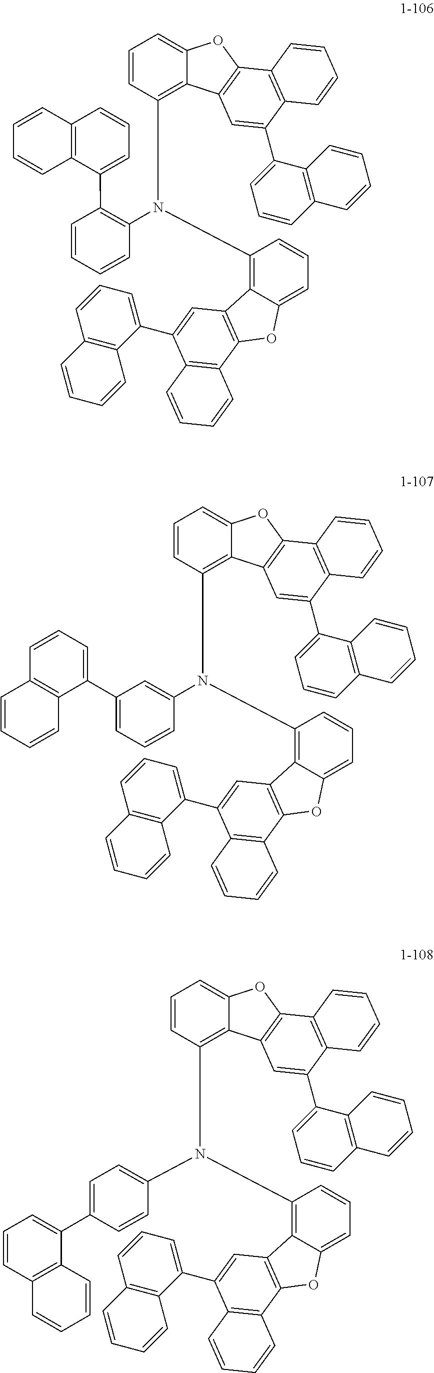 [SMILES: c1ccc(N(c2cccc3oc4c5ccccc5c(-c5cccc6ccccc56)cc4c23)c2cccc3oc4c5ccccc5c(-c5cccc6ccccc56)cc4c23)c(-c2cccc3ccccc23)c1 ; c1cc(-c2cccc3ccccc23)cc(N(c2cccc3oc4c5ccccc5c(-c5cccc6ccccc56)cc4c23)c2cccc3oc4c5ccccc5c(-c5cccc6ccccc56)cc4c23)c1 ; c1ccc2c(-c3ccc(N(c4cccc5oc6c7ccccc7c(-c7cccc8ccccc78)cc6c45)c4cccc5oc6c7ccccc7c(-c7cccc8ccccc78)cc6c45)cc3)cccc2c1]

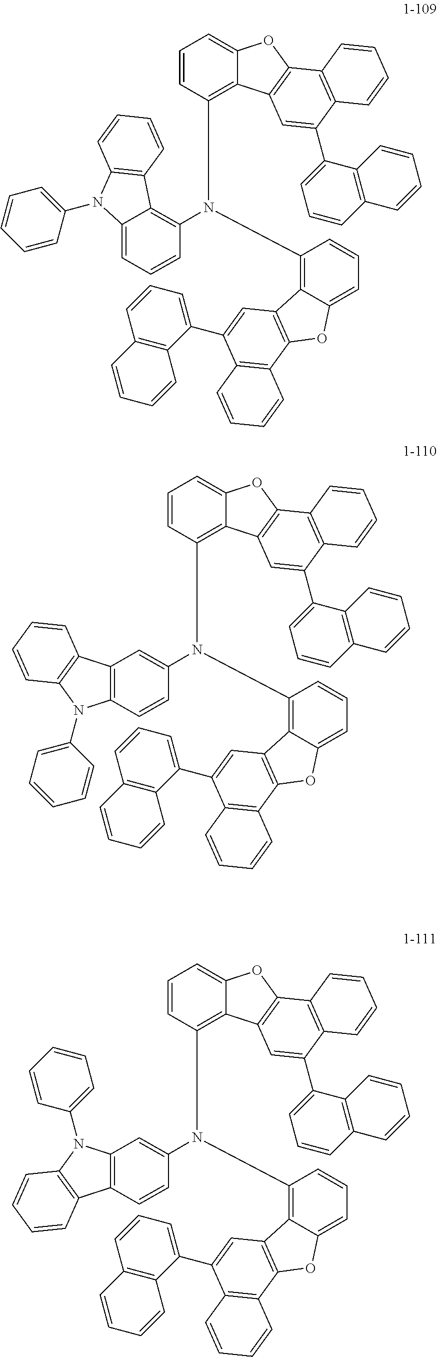 [SMILES: c1ccc(-n2c3ccccc3c3c(N(c4cccc5oc6c7ccccc7c(-c7cccc8ccccc78)cc6c45)c4cccc5oc6c7ccccc7c(-c7cccc8ccccc78)cc6c45)cccc32)cc1 ; c1ccc(-n2c3ccccc3c3cc(N(c4cccc5oc6c7ccccc7c(-c7cccc8ccccc78)cc6c45)c4cccc5oc6c7ccccc7c(-c7cccc8ccccc78)cc6c45)ccc32)cc1 ; c1ccc(-n2c3ccccc3c3ccc(N(c4cccc5oc6c7ccccc7c(-c7cccc8ccccc78)cc6c45)c4cccc5oc6c7ccccc7c(-c7cccc8ccccc78)cc6c45)cc32)cc1]

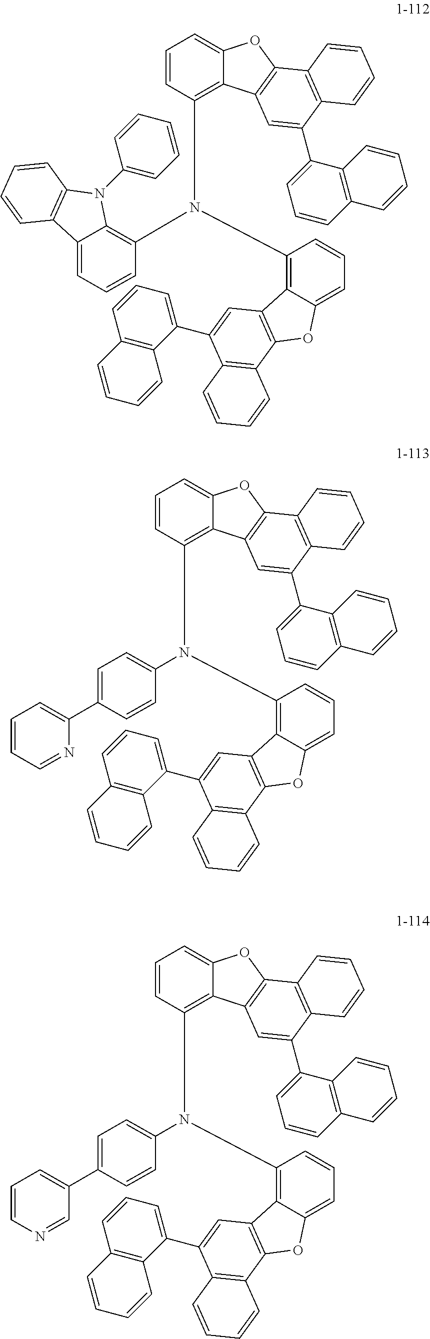 [SMILES: c1ccc(-n2c3ccccc3c3cccc(N(c4cccc5oc6c7ccccc7c(-c7cccc8ccccc78)cc6c45)c4cccc5oc6c7ccccc7c(-c7cccc8ccccc78)cc6c45)c32)cc1 ; c1ccc(-c2ccc(N(c3cccc4oc5c6ccccc6c(-c6cccc7ccccc67)cc5c34)c3cccc4oc5c6ccccc6c(-c6cccc7ccccc67)cc5c34)cc2)nc1 ; c1cncc(-c2ccc(N(c3cccc4oc5c6ccccc6c(-c6cccc7ccccc67)cc5c34)c3cccc4oc5c6ccccc6c(-c6cccc7ccccc67)cc5c34)cc2)c1]

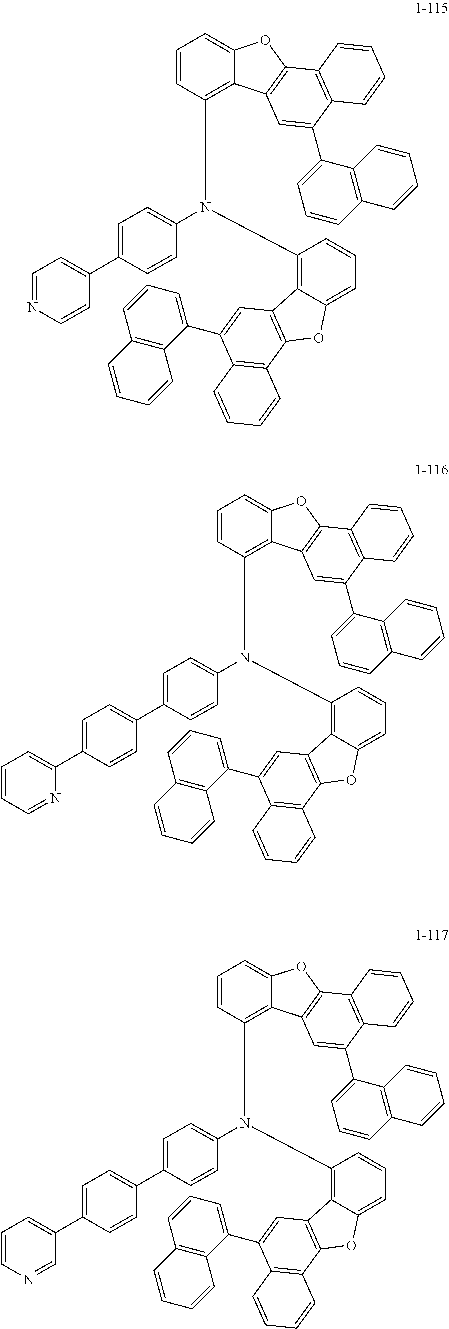 [SMILES: c1ccc2c(-c3cc4c(oc5cccc(N(c6ccc(-c7ccncc7)cc6)c6cccc7oc8c9ccccc9c(-c9cccc%10ccccc9%10)cc8c67)c54)c4ccccc34)cccc2c1 ; c1ccc(-c2ccc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2)nc1 ; c1cncc(-c2ccc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2)c1]

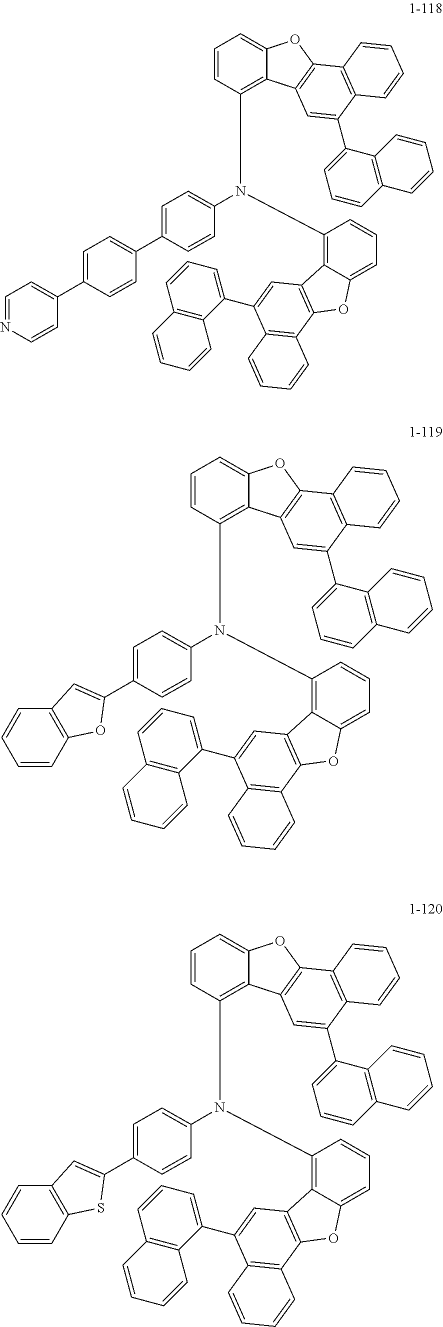 [SMILES: c1ccc2c(-c3cc4c(oc5cccc(N(c6ccc(-c7ccc(-c8ccncc8)cc7)cc6)c6cccc7oc8c9ccccc9c(-c9cccc%10ccccc9%10)cc8c67)c54)c4ccccc34)cccc2c1 ; c1ccc2oc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2c1 ; c1ccc2sc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2c1]

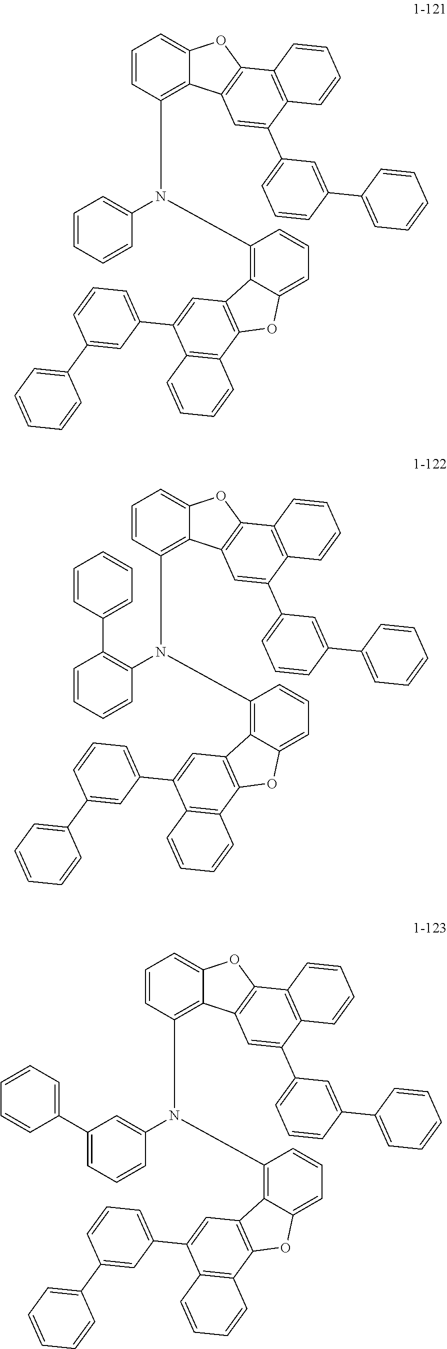 [SMILES: c1ccc(-c2cccc(-c3cc4c(oc5cccc(N(c6ccccc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccccc6-c6ccccc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc(-c7ccccc7)c6)c6cccc7oc8c9ccccc9c(-c9cccc(-c%10ccccc%10)c9)cc8c67)c54)c4ccccc34)c2)cc1]

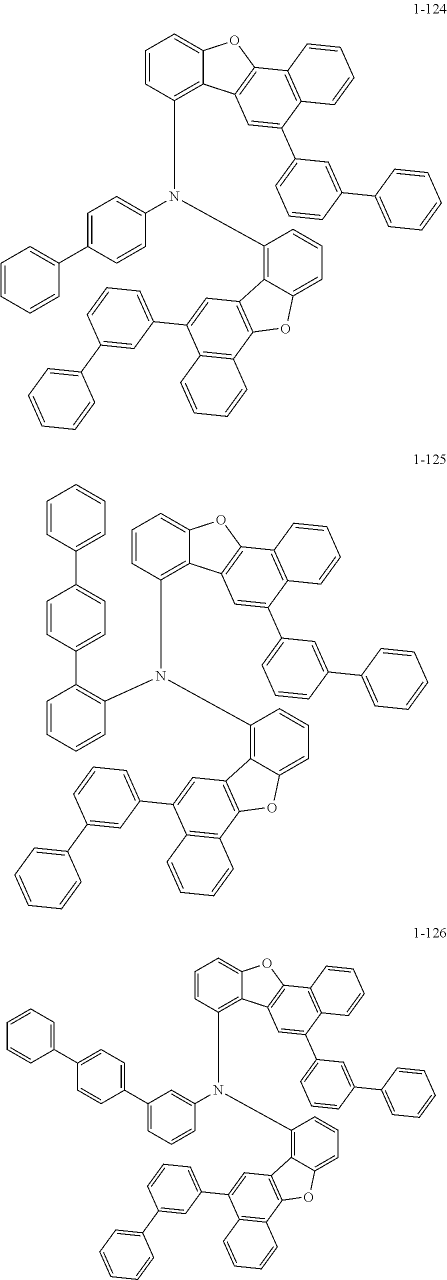 [SMILES: c1ccc(-c2ccc(N(c3cccc4oc5c6ccccc6c(-c6cccc(-c7ccccc7)c6)cc5c34)c3cccc4oc5c6ccccc6c(-c6cccc(-c7ccccc7)c6)cc5c34)cc2)cc1 ; c1ccc(-c2ccc(-c3ccccc3N(c3cccc4oc5c6ccccc6c(-c6cccc(-c7ccccc7)c6)cc5c34)c3cccc4oc5c6ccccc6c(-c6cccc(-c7ccccc7)c6)cc5c34)cc2)cc1 ; c1ccc(-c2ccc(-c3cccc(N(c4cccc5oc6c7ccccc7c(-c7cccc(-c8ccccc8)c7)cc6c45)c4cccc5oc6c7ccccc7c(-c7cccc(-c8ccccc8)c7)cc6c45)c3)cc2)cc1]

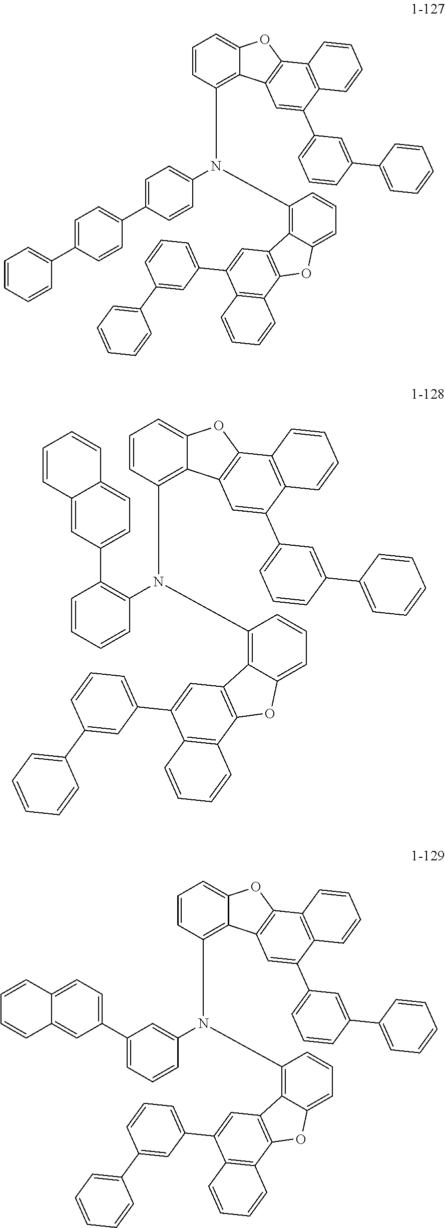 [SMILES: c1ccc(-c2ccc(-c3ccc(N(c4cccc5oc6c7ccccc7c(-c7cccc(-c8ccccc8)c7)cc6c45)c4cccc5oc6c7ccccc7c(-c7cccc(-c8ccccc8)c7)cc6c45)cc3)cc2)cc1 ; c1ccc(-c2cccc(-c3cc4c(oc5cccc(N(c6ccccc6-c6ccc7ccccc7c6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc(-c7ccc8ccccc8c7)c6)c6cccc7oc8c9ccccc9c(-c9cccc(-c%10ccccc%10)c9)cc8c67)c54)c4ccccc34)c2)cc1]

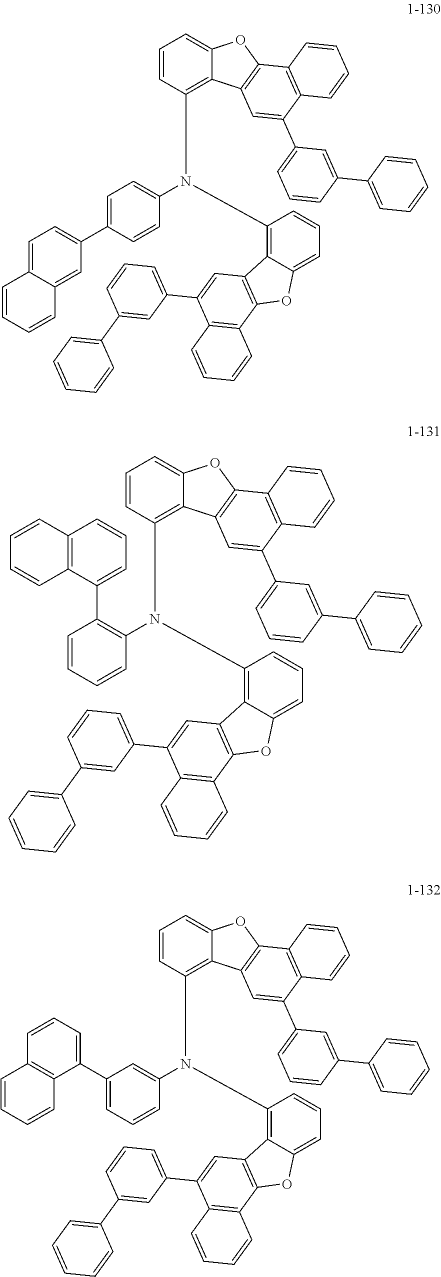 [SMILES: c1ccc(-c2cccc(-c3cc4c(oc5cccc(N(c6ccc(-c7ccc8ccccc8c7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccccc6-c6cccc7ccccc67)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc(-c7cccc8ccccc78)c6)c6cccc7oc8c9ccccc9c(-c9cccc(-c%10ccccc%10)c9)cc8c67)c54)c4ccccc34)c2)cc1]

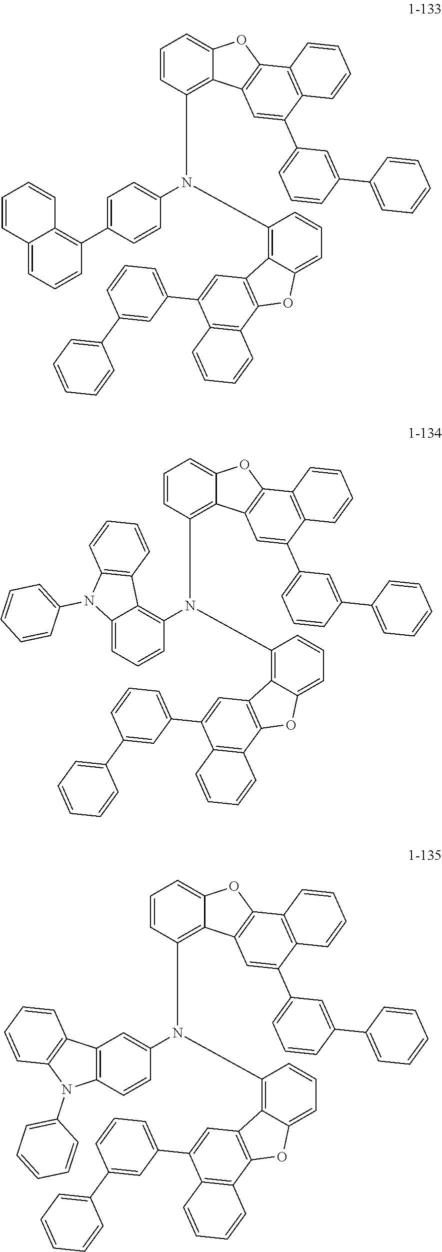 [SMILES: c1ccc(-c2cccc(-c3cc4c(oc5cccc(N(c6ccc(-c7cccc8ccccc78)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc7oc8c9ccccc9c(-c9cccc(-c%10ccccc%10)c9)cc8c67)c6cccc7c6c6ccccc6n7-c6ccccc6)c54)c4ccccc34)c2)cc1 ; c1ccc(-c2cccc(-c3cc4c(oc5cccc(N(c6ccc7c(c6)c6ccccc6n7-c6ccccc6)c6cccc7oc8c9ccccc9c(-c9cccc(-c%10ccccc%10)c9)cc8c67)c54)c4ccccc34)c2)cc1]

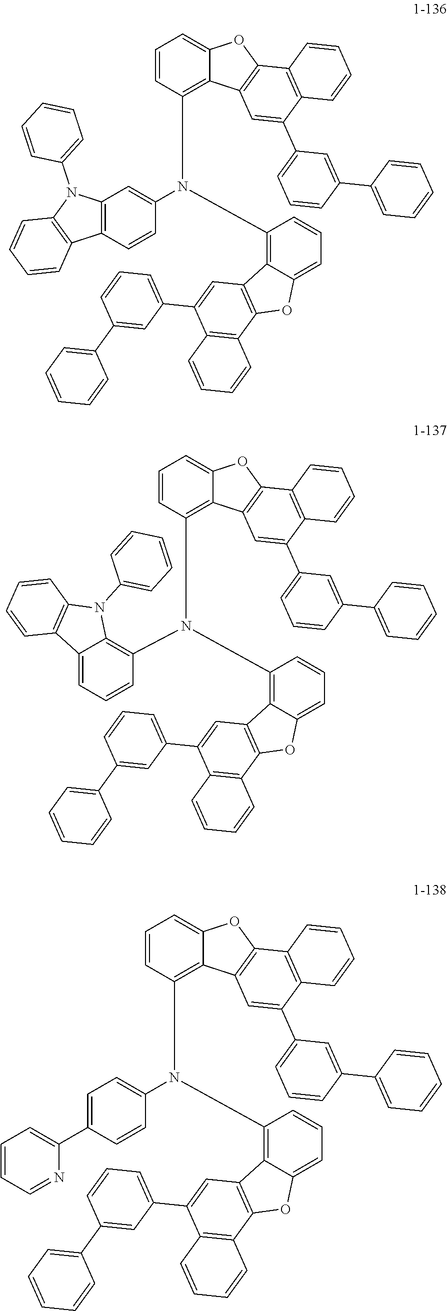 [SMILES: c1ccc(-c2cccc(-c3cc4c(oc5cccc(N(c6ccc7c8ccccc8n(-c8ccccc8)c7c6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc7oc8c9ccccc9c(-c9cccc(-c%10ccccc%10)c9)cc8c67)c6cccc7c8ccccc8n(-c8ccccc8)c67)c54)c4ccccc34)c2)cc1 ; c1ccc(-c2cccc(-c3cc4c(oc5cccc(N(c6ccc(-c7ccccn7)cc6)c6cccc7oc8c9ccccc9c(-c9cccc(-c%10ccccc%10)c9)cc8c67)c54)c4ccccc34)c2)cc1]

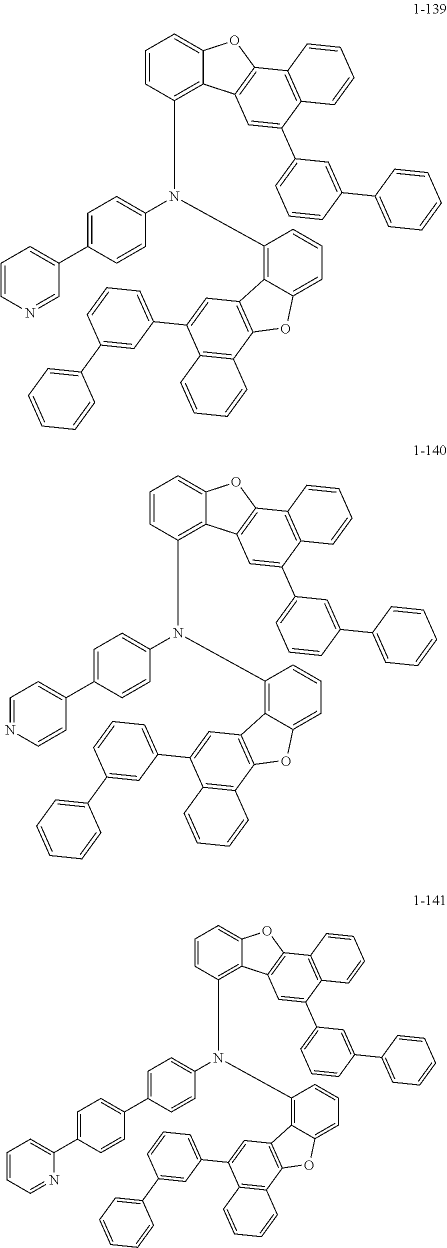 [SMILES: c1ccc(-c2cccc(-c3cc4c(oc5cccc(N(c6ccc(-c7cccnc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7ccncc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7ccc(-c8ccccn8)cc7)cc6)c6cccc7oc8c9ccccc9c(-c9cccc(-c%10ccccc%10)c9)cc8c67)c54)c4ccccc34)c2)cc1]

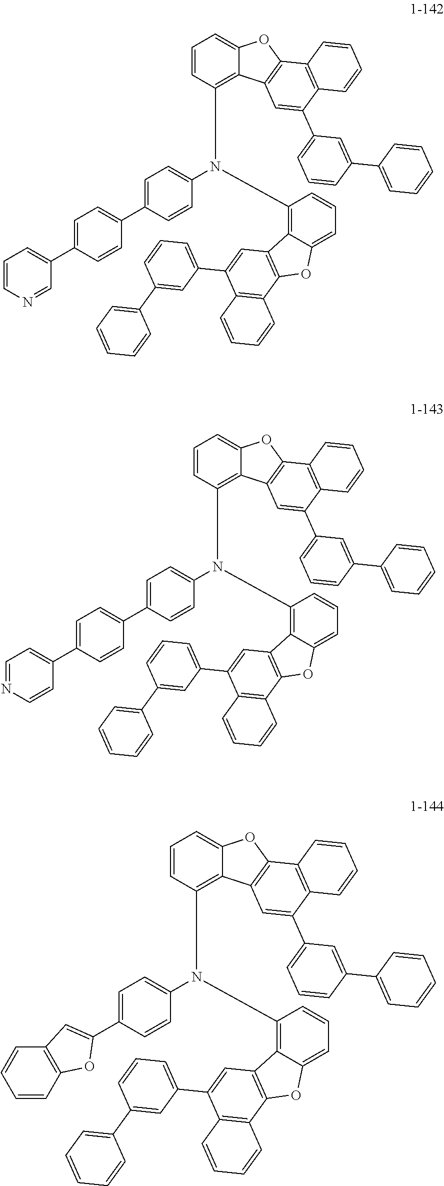 [SMILES: c1ccc(-c2cccc(-c3cc4c(oc5cccc(N(c6ccc(-c7ccc(-c8cccnc8)cc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7ccc(-c8ccncc8)cc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7cc8ccccc8o7)cc6)c6cccc7oc8c9ccccc9c(-c9cccc(-c%10ccccc%10)c9)cc8c67)c54)c4ccccc34)c2)cc1]

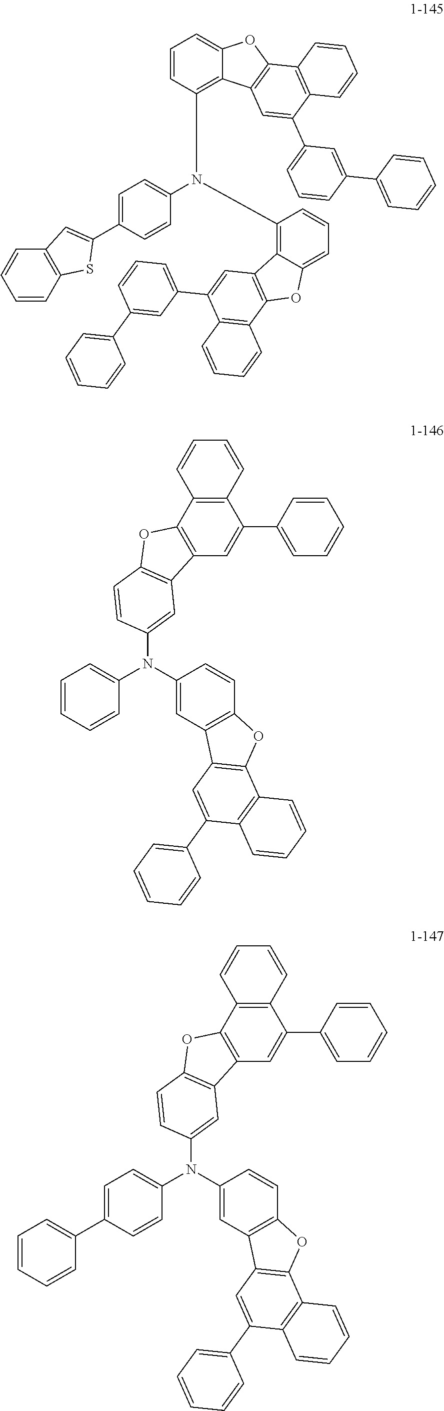 [SMILES: c1ccc(-c2cccc(-c3cc4c(oc5cccc(N(c6ccc(-c7cc8ccccc8s7)cc6)c6cccc7oc8c9ccccc9c(-c9cccc(-c%10ccccc%10)c9)cc8c67)c54)c4ccccc34)c2)cc1 ; c1ccc(-c2cc3c4cc(N(c5ccccc5)c5ccc6oc7c8ccccc8c(-c8ccccc8)cc7c6c5)ccc4oc3c3ccccc23)cc1 ; c1ccc(-c2ccc(N(c3ccc4oc5c6ccccc6c(-c6ccccc6)cc5c4c3)c3ccc4oc5c6ccccc6c(-c6ccccc6)cc5c4c3)cc2)cc1]

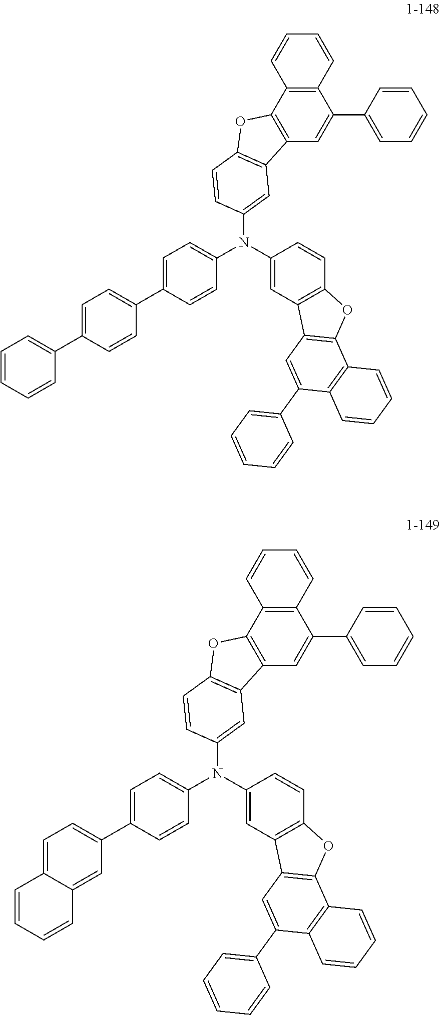 [SMILES: c1ccc(-c2ccc(-c3ccc(N(c4ccc5oc6c7ccccc7c(-c7ccccc7)cc6c5c4)c4ccc5oc6c7ccccc7c(-c7ccccc7)cc6c5c4)cc3)cc2)cc1 ; c1ccc(-c2cc3c4cc(N(c5ccc(-c6ccc7ccccc7c6)cc5)c5ccc6oc7c8ccccc8c(-c8ccccc8)cc7c6c5)ccc4oc3c3ccccc23)cc1]

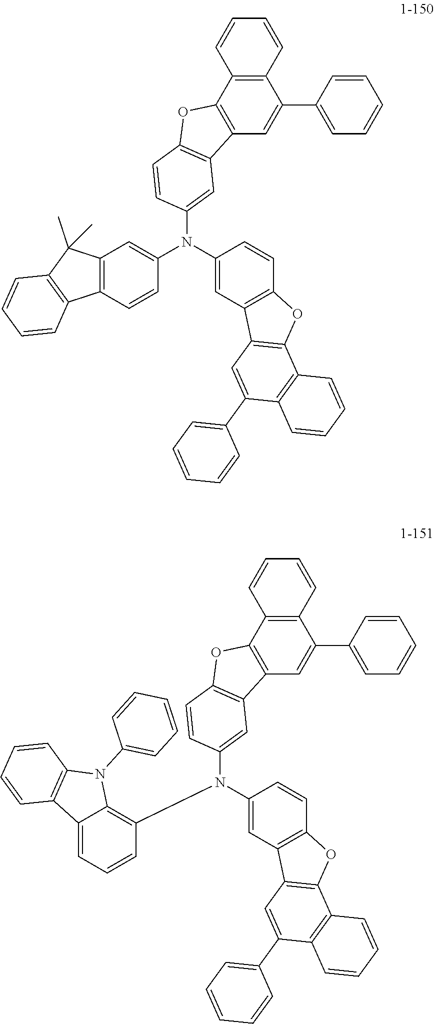 [SMILES: CC1(C)c2ccccc2-c2ccc(N(c3ccc4oc5c6ccccc6c(-c6ccccc6)cc5c4c3)c3ccc4oc5c6ccccc6c(-c6ccccc6)cc5c4c3)cc21 ; c1ccc(-c2cc3c4cc(N(c5ccc6oc7c8ccccc8c(-c8ccccc8)cc7c6c5)c5cccc6c7ccccc7n(-c7ccccc7)c56)ccc4oc3c3ccccc23)cc1]

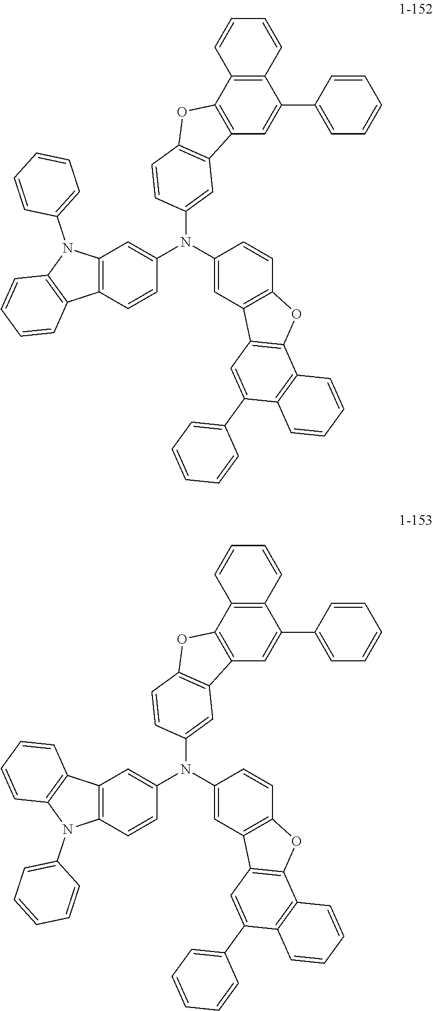 [SMILES: c1ccc(-c2cc3c4cc(N(c5ccc6oc7c8ccccc8c(-c8ccccc8)cc7c6c5)c5ccc6c7ccccc7n(-c7ccccc7)c6c5)ccc4oc3c3ccccc23)cc1 ; c1ccc(-c2cc3c4cc(N(c5ccc6oc7c8ccccc8c(-c8ccccc8)cc7c6c5)c5ccc6c(c5)c5ccccc5n6-c5ccccc5)ccc4oc3c3ccccc23)cc1]

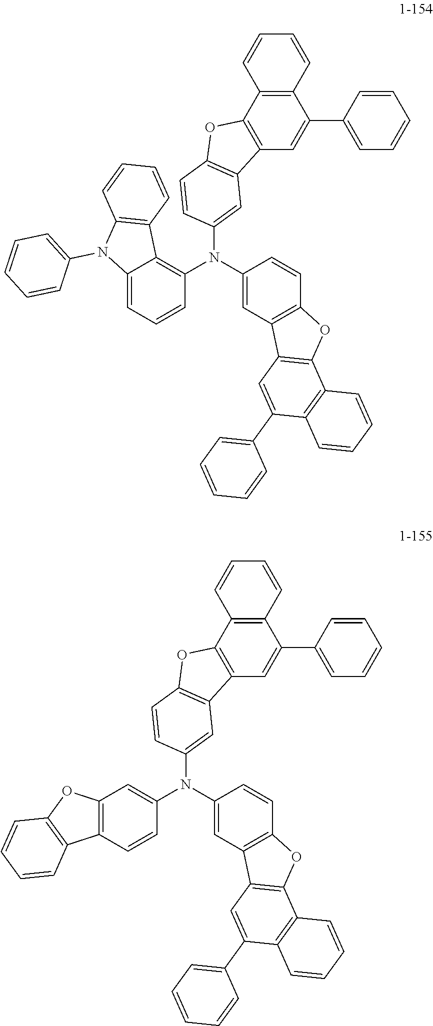 [SMILES: c1ccc(-c2cc3c4cc(N(c5ccc6oc7c8ccccc8c(-c8ccccc8)cc7c6c5)c5cccc6c5c5ccccc5n6-c5ccccc5)ccc4oc3c3ccccc23)cc1 ; c1ccc(-c2cc3c4cc(N(c5ccc6c(c5)oc5ccccc56)c5ccc6oc7c8ccccc8c(-c8ccccc8)cc7c6c5)ccc4oc3c3ccccc23)cc1]

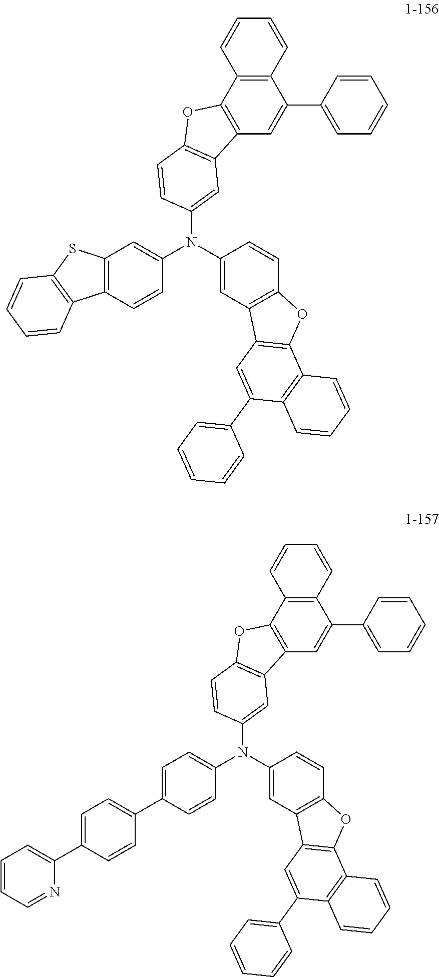 [SMILES: c1ccc(-c2cc3c4cc(N(c5ccc6c(c5)sc5ccccc56)c5ccc6oc7c8ccccc8c(-c8ccccc8)cc7c6c5)ccc4oc3c3ccccc23)cc1 ; c1ccc(-c2cc3c4cc(N(c5ccc(-c6ccc(-c7ccccn7)cc6)cc5)c5ccc6oc7c8ccccc8c(-c8ccccc8)cc7c6c5)ccc4oc3c3ccccc23)cc1]

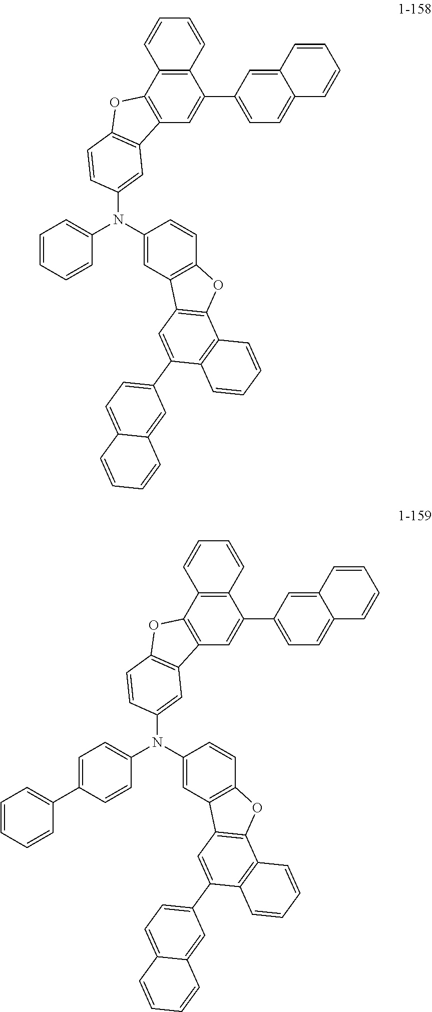 [SMILES: c1ccc(N(c2ccc3oc4c5ccccc5c(-c5ccc6ccccc6c5)cc4c3c2)c2ccc3oc4c5ccccc5c(-c5ccc6ccccc6c5)cc4c3c2)cc1 ; c1ccc(-c2ccc(N(c3ccc4oc5c6ccccc6c(-c6ccc7ccccc7c6)cc5c4c3)c3ccc4oc5c6ccccc6c(-c6ccc7ccccc7c6)cc5c4c3)cc2)cc1]

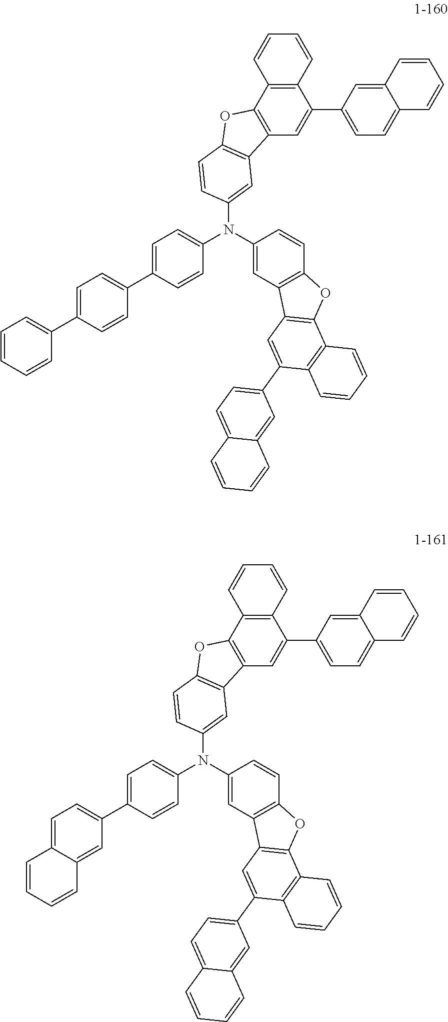 [SMILES: c1ccc(-c2ccc(-c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc3)cc2)cc1 ; c1ccc2cc(-c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc3)ccc2c1]

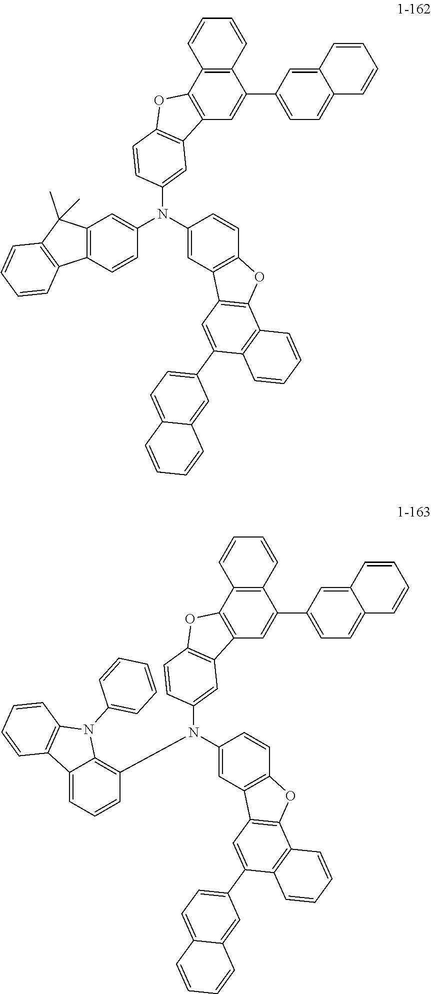 [SMILES: CC1(C)c2ccccc2-c2ccc(N(c3ccc4oc5c6ccccc6c(-c6ccc7ccccc7c6)cc5c4c3)c3ccc4oc5c6ccccc6c(-c6ccc7ccccc7c6)cc5c4c3)cc21 ; c1ccc(-n2c3ccccc3c3cccc(N(c4ccc5oc6c7ccccc7c(-c7ccc8ccccc8c7)cc6c5c4)c4ccc5oc6c7ccccc7c(-c7ccc8ccccc8c7)cc6c5c4)c32)cc1]

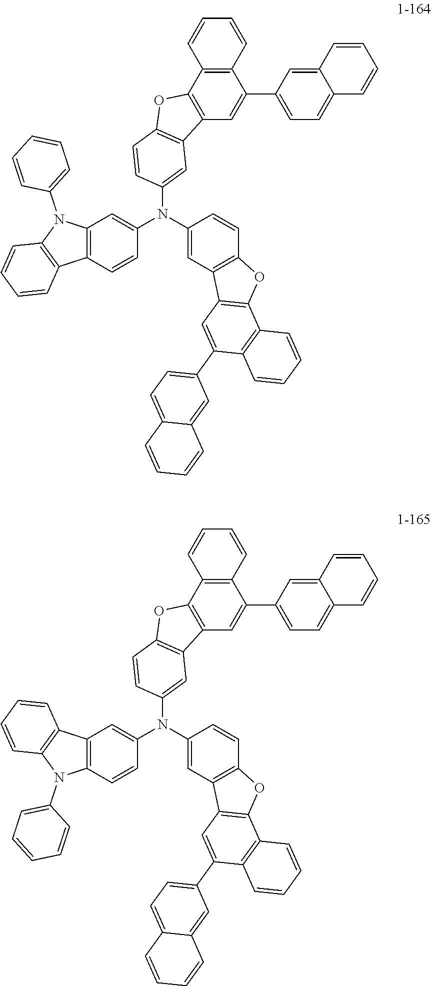 [SMILES: c1ccc(-n2c3ccccc3c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc32)cc1 ; c1ccc(-n2c3ccccc3c3cc(N(c4ccc5oc6c7ccccc7c(-c7ccc8ccccc8c7)cc6c5c4)c4ccc5oc6c7ccccc7c(-c7ccc8ccccc8c7)cc6c5c4)ccc32)cc1]

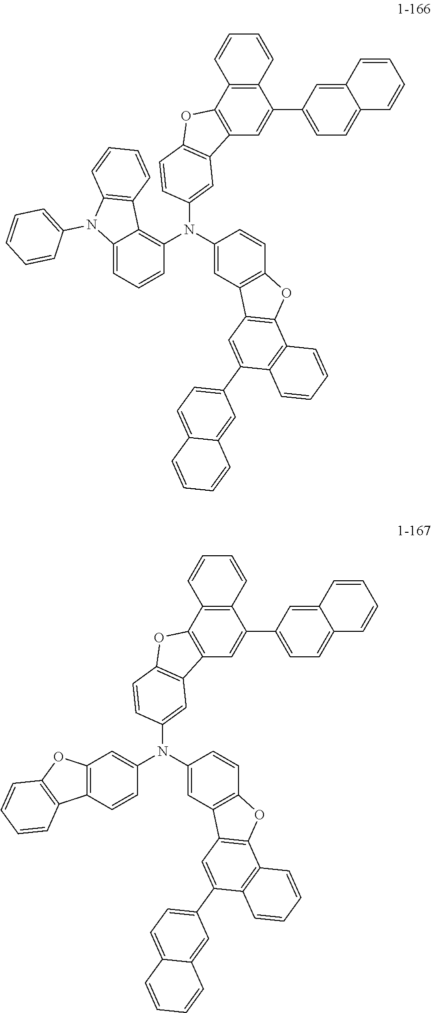 [SMILES: c1ccc(-n2c3ccccc3c3c(N(c4ccc5oc6c7ccccc7c(-c7ccc8ccccc8c7)cc6c5c4)c4ccc5oc6c7ccccc7c(-c7ccc8ccccc8c7)cc6c5c4)cccc32)cc1 ; c1ccc2cc(-c3cc4c5cc(N(c6ccc7c(c6)oc6ccccc67)c6ccc7oc8c9ccccc9c(-c9ccc%10ccccc%10c9)cc8c7c6)ccc5oc4c4ccccc34)ccc2c1]

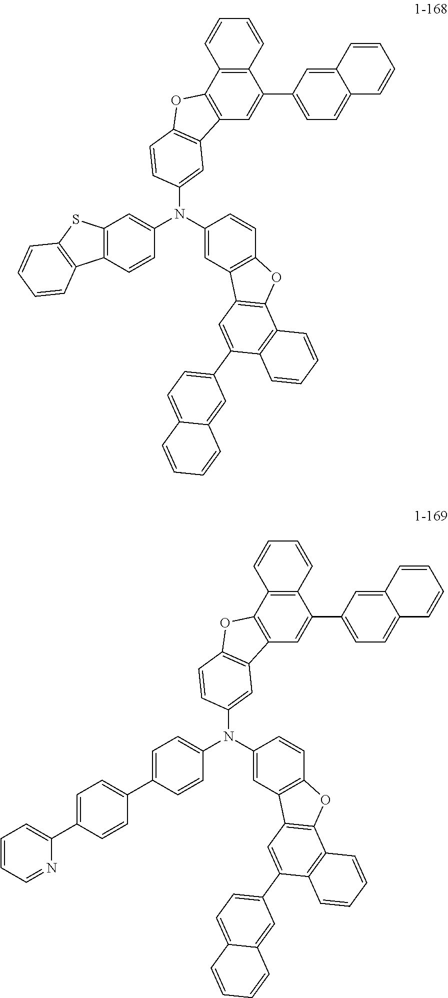 [SMILES: c1ccc2cc(-c3cc4c5cc(N(c6ccc7c(c6)sc6ccccc67)c6ccc7oc8c9ccccc9c(-c9ccc%10ccccc%10c9)cc8c7c6)ccc5oc4c4ccccc34)ccc2c1 ; c1ccc(-c2ccc(-c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc3)cc2)nc1]

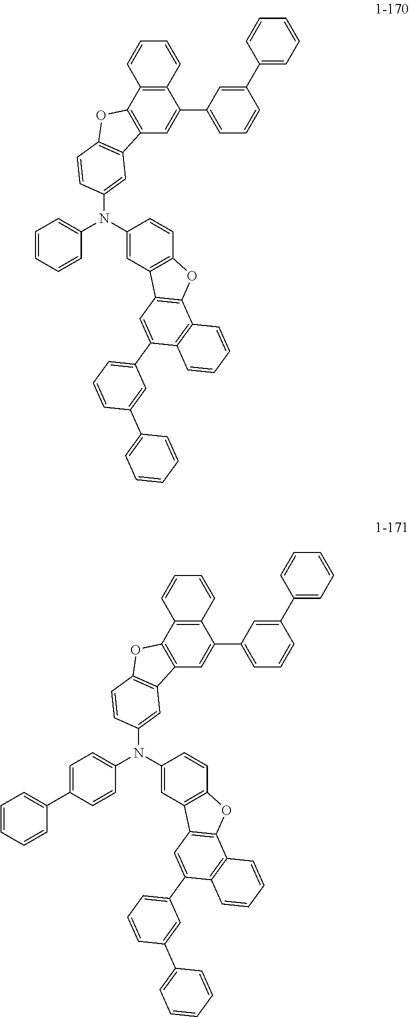 [SMILES: c1ccc(-c2cccc(-c3cc4c5cc(N(c6ccccc6)c6ccc7oc8c9ccccc9c(-c9cccc(-c%10ccccc%10)c9)cc8c7c6)ccc5oc4c4ccccc34)c2)cc1 ; c1ccc(-c2ccc(N(c3ccc4oc5c6ccccc6c(-c6cccc(-c7ccccc7)c6)cc5c4c3)c3ccc4oc5c6ccccc6c(-c6cccc(-c7ccccc7)c6)cc5c4c3)cc2)cc1]

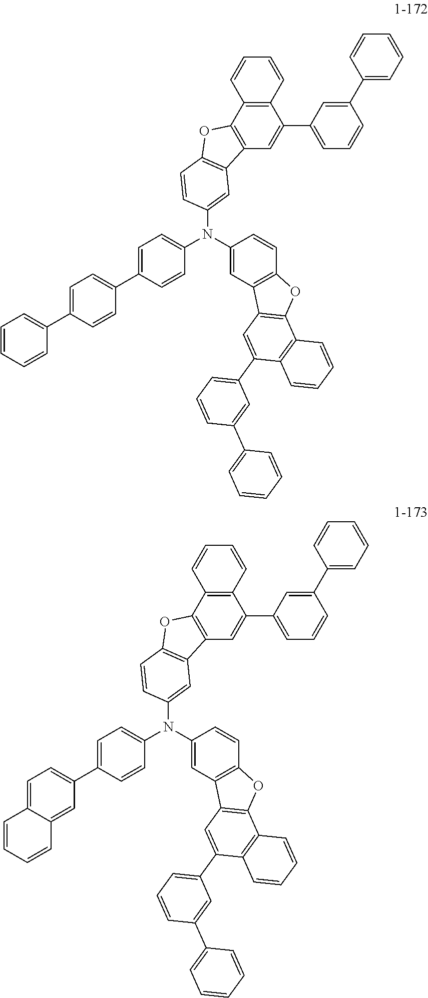 [SMILES: c1ccc(-c2ccc(-c3ccc(N(c4ccc5oc6c7ccccc7c(-c7cccc(-c8ccccc8)c7)cc6c5c4)c4ccc5oc6c7ccccc7c(-c7cccc(-c8ccccc8)c7)cc6c5c4)cc3)cc2)cc1 ; c1ccc(-c2cccc(-c3cc4c5cc(N(c6ccc(-c7ccc8ccccc8c7)cc6)c6ccc7oc8c9ccccc9c(-c9cccc(-c%10ccccc%10)c9)cc8c7c6)ccc5oc4c4ccccc34)c2)cc1]

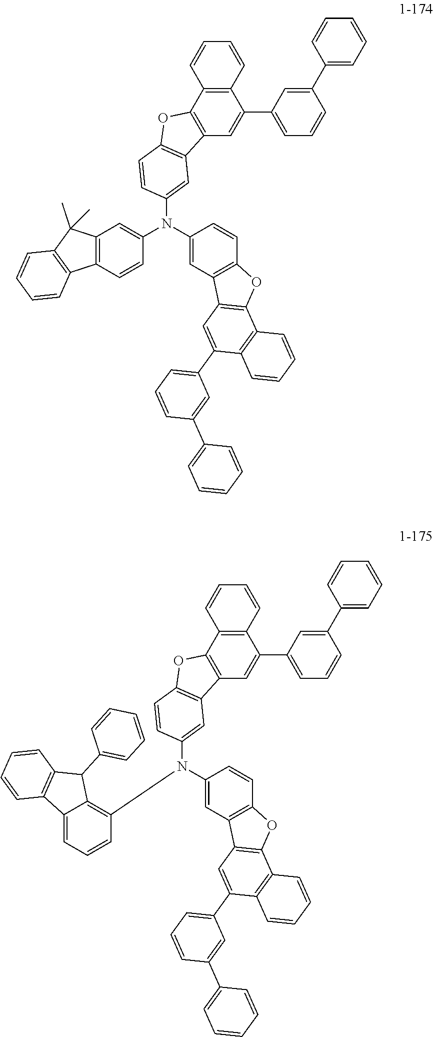 [SMILES: CC1(C)c2ccccc2-c2ccc(N(c3ccc4oc5c6ccccc6c(-c6cccc(-c7ccccc7)c6)cc5c4c3)c3ccc4oc5c6ccccc6c(-c6cccc(-c7ccccc7)c6)cc5c4c3)cc21 ; c1ccc(-c2cccc(-c3cc4c5cc(N(c6ccc7oc8c9ccccc9c(-c9cccc(-c%10ccccc%10)c9)cc8c7c6)c6cccc7c6C(c6ccccc6)c6ccccc6-7)ccc5oc4c4ccccc34)c2)cc1]

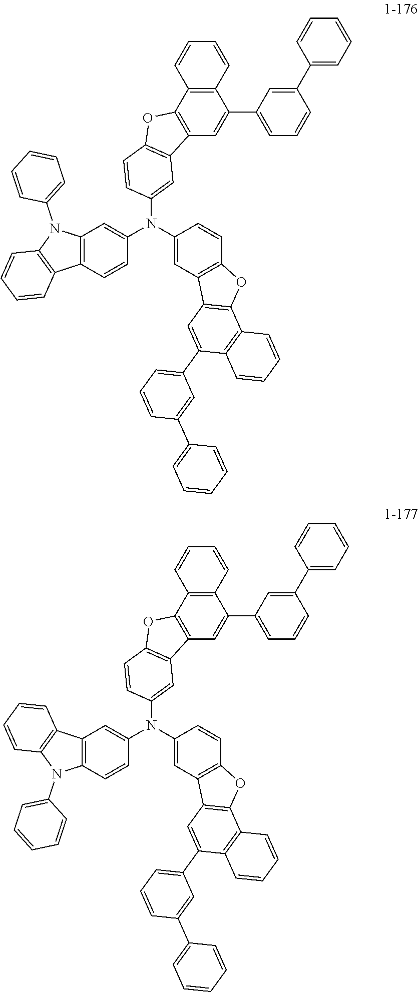 [SMILES: c1ccc(-c2cccc(-c3cc4c5cc(N(c6ccc7oc8c9ccccc9c(-c9cccc(-c%10ccccc%10)c9)cc8c7c6)c6ccc7c8ccccc8n(-c8ccccc8)c7c6)ccc5oc4c4ccccc34)c2)cc1 ; c1ccc(-c2cccc(-c3cc4c5cc(N(c6ccc7oc8c9ccccc9c(-c9cccc(-c%10ccccc%10)c9)cc8c7c6)c6ccc7c(c6)c6ccccc6n7-c6ccccc6)ccc5oc4c4ccccc34)c2)cc1]

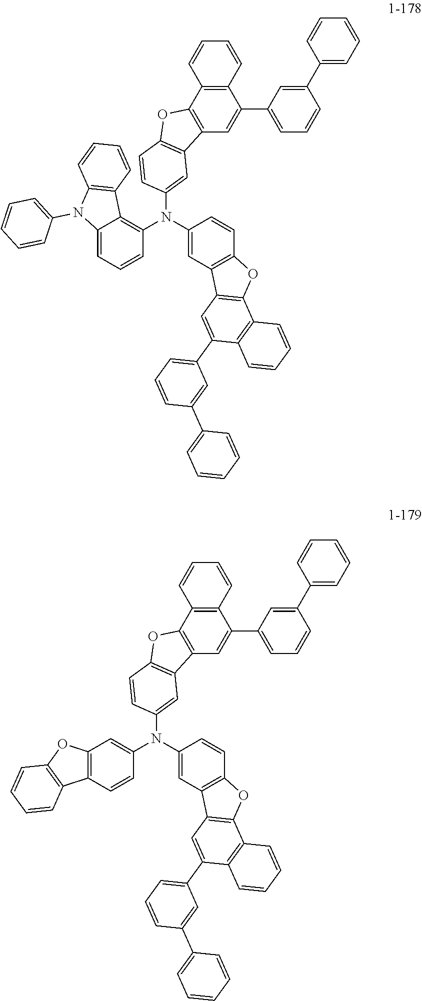 [SMILES: c1ccc(-c2cccc(-c3cc4c5cc(N(c6ccc7oc8c9ccccc9c(-c9cccc(-c%10ccccc%10)c9)cc8c7c6)c6cccc7c6c6ccccc6n7-c6ccccc6)ccc5oc4c4ccccc34)c2)cc1 ; c1ccc(-c2cccc(-c3cc4c5cc(N(c6ccc7c(c6)oc6ccccc67)c6ccc7oc8c9ccccc9c(-c9cccc(-c%10ccccc%10)c9)cc8c7c6)ccc5oc4c4ccccc34)c2)cc1]

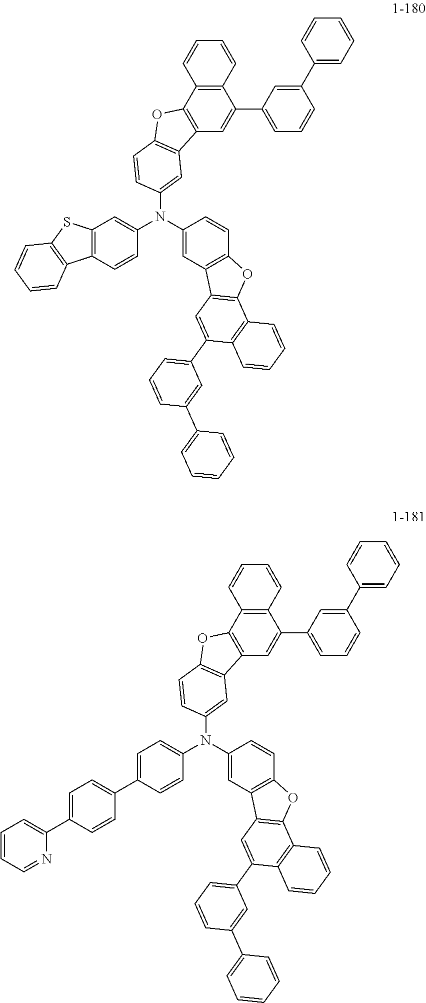 [SMILES: c1ccc(-c2cccc(-c3cc4c5cc(N(c6ccc7c(c6)sc6ccccc67)c6ccc7oc8c9ccccc9c(-c9cccc(-c%10ccccc%10)c9)cc8c7c6)ccc5oc4c4ccccc34)c2)cc1 ; c1ccc(-c2cccc(-c3cc4c5cc(N(c6ccc(-c7ccc(-c8ccccn8)cc7)cc6)c6ccc7oc8c9ccccc9c(-c9cccc(-c%10ccccc%10)c9)cc8c7c6)ccc5oc4c4ccccc34)c2)cc1]

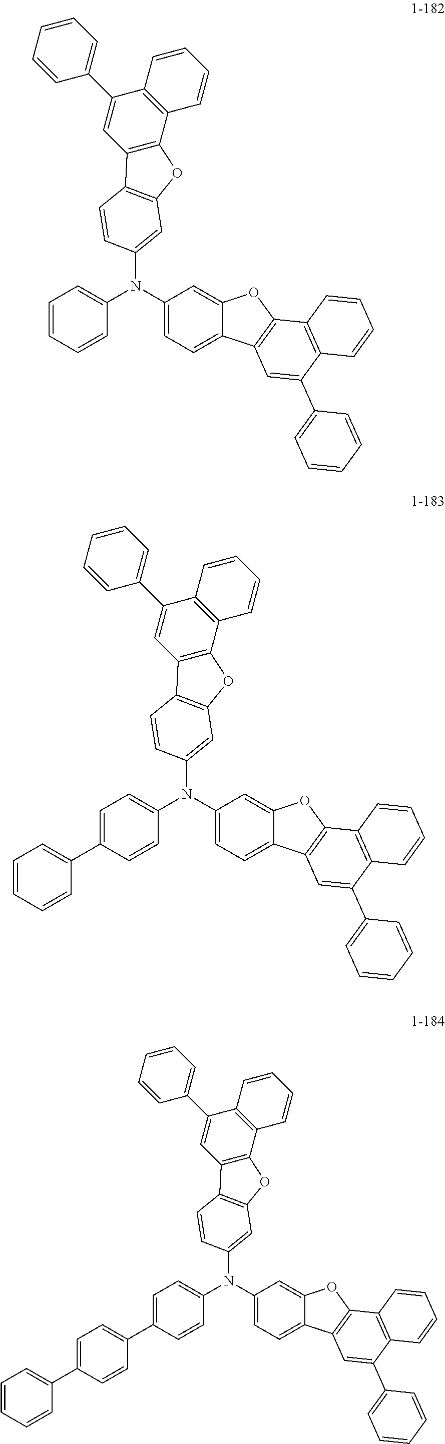 [SMILES: c1ccc(-c2cc3c4ccc(N(c5ccccc5)c5ccc6c(c5)oc5c7ccccc7c(-c7ccccc7)cc65)cc4oc3c3ccccc23)cc1 ; c1ccc(-c2ccc(N(c3ccc4c(c3)oc3c5ccccc5c(-c5ccccc5)cc43)c3ccc4c(c3)oc3c5ccccc5c(-c5ccccc5)cc43)cc2)cc1 ; c1ccc(-c2ccc(-c3ccc(N(c4ccc5c(c4)oc4c6ccccc6c(-c6ccccc6)cc54)c4ccc5c(c4)oc4c6ccccc6c(-c6ccccc6)cc54)cc3)cc2)cc1]

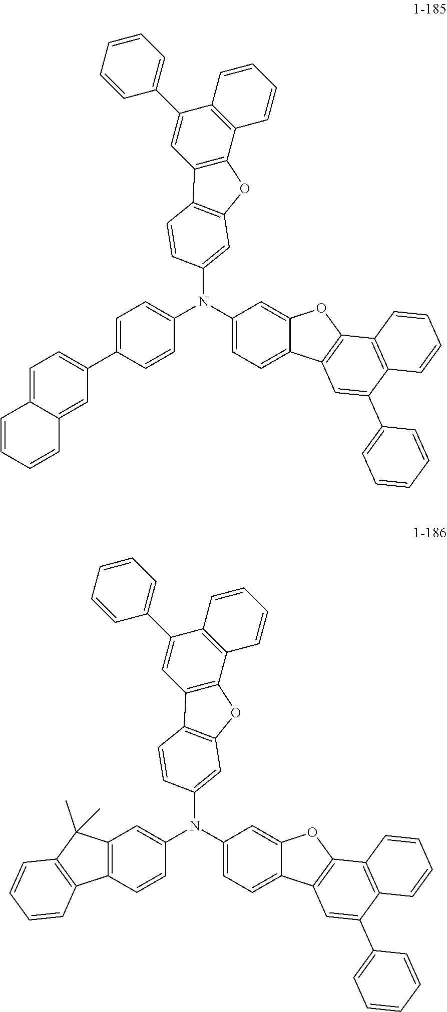 [SMILES: c1ccc(-c2cc3c4ccc(N(c5ccc(-c6ccc7ccccc7c6)cc5)c5ccc6c(c5)oc5c7ccccc7c(-c7ccccc7)cc65)cc4oc3c3ccccc23)cc1 ; CC1(C)c2ccccc2-c2ccc(N(c3ccc4c(c3)oc3c5ccccc5c(-c5ccccc5)cc43)c3ccc4c(c3)oc3c5ccccc5c(-c5ccccc5)cc43)cc21]

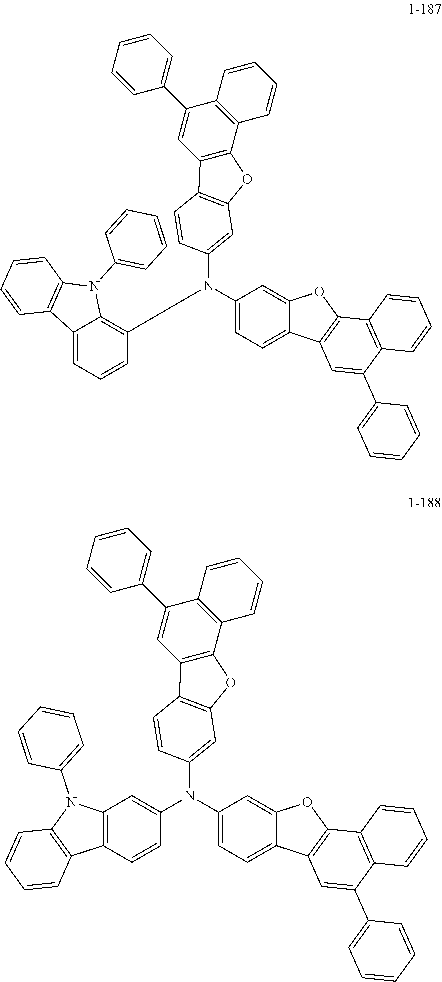 [SMILES: c1ccc(-c2cc3c4ccc(N(c5ccc6c(c5)oc5c7ccccc7c(-c7ccccc7)cc65)c5cccc6c7ccccc7n(-c7ccccc7)c56)cc4oc3c3ccccc23)cc1 ; c1ccc(-c2cc3c4ccc(N(c5ccc6c(c5)oc5c7ccccc7c(-c7ccccc7)cc65)c5ccc6c7ccccc7n(-c7ccccc7)c6c5)cc4oc3c3ccccc23)cc1]

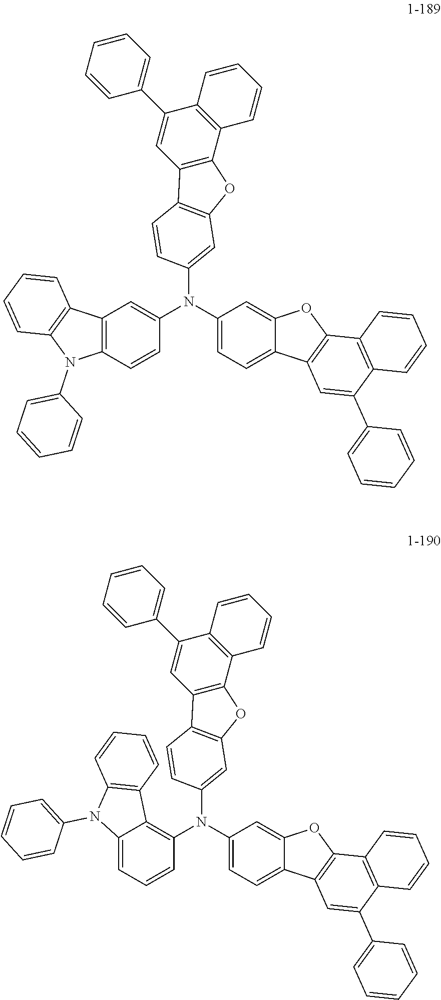 [SMILES: c1ccc(-c2cc3c4ccc(N(c5ccc6c(c5)oc5c7ccccc7c(-c7ccccc7)cc65)c5ccc6c(c5)c5ccccc5n6-c5ccccc5)cc4oc3c3ccccc23)cc1 ; c1ccc(-c2cc3c4ccc(N(c5ccc6c(c5)oc5c7ccccc7c(-c7ccccc7)cc65)c5cccc6c5c5ccccc5n6-c5ccccc5)cc4oc3c3ccccc23)cc1]

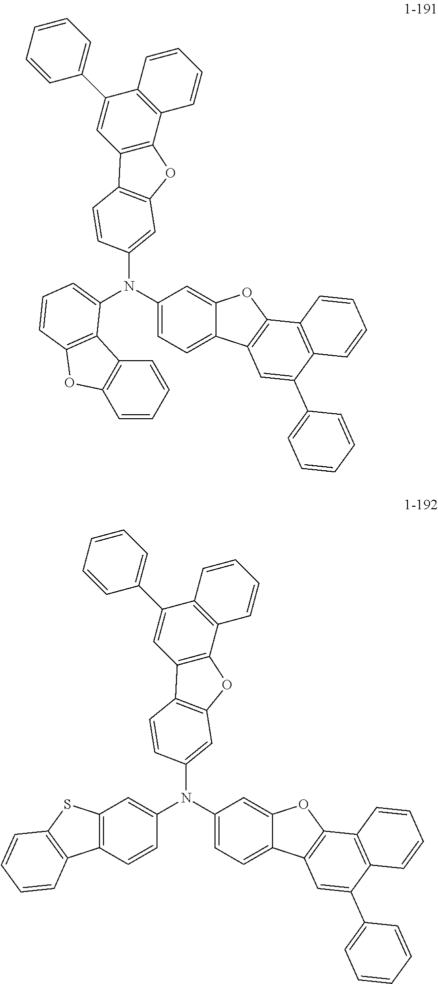 [SMILES: c1ccc(-c2cc3c4ccc(N(c5ccc6c(c5)oc5c7ccccc7c(-c7ccccc7)cc65)c5cccc6oc7ccccc7c56)cc4oc3c3ccccc23)cc1 ; c1ccc(-c2cc3c4ccc(N(c5ccc6c(c5)oc5c7ccccc7c(-c7ccccc7)cc65)c5ccc6c(c5)sc5ccccc56)cc4oc3c3ccccc23)cc1]

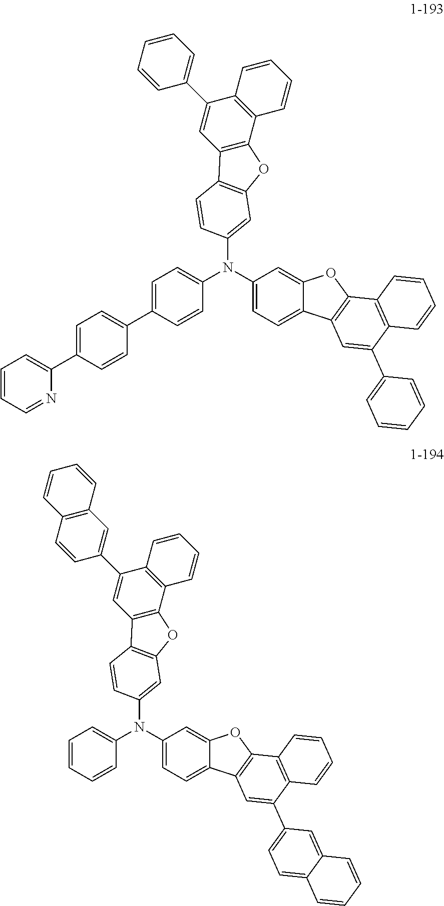 [SMILES: c1ccc(-c2cc3c4ccc(N(c5ccc(-c6ccc(-c7ccccn7)cc6)cc5)c5ccc6c(c5)oc5c7ccccc7c(-c7ccccc7)cc65)cc4oc3c3ccccc23)cc1 ; c1ccc(N(c2ccc3c(c2)oc2c4ccccc4c(-c4ccc5ccccc5c4)cc32)c2ccc3c(c2)oc2c4ccccc4c(-c4ccc5ccccc5c4)cc32)cc1]

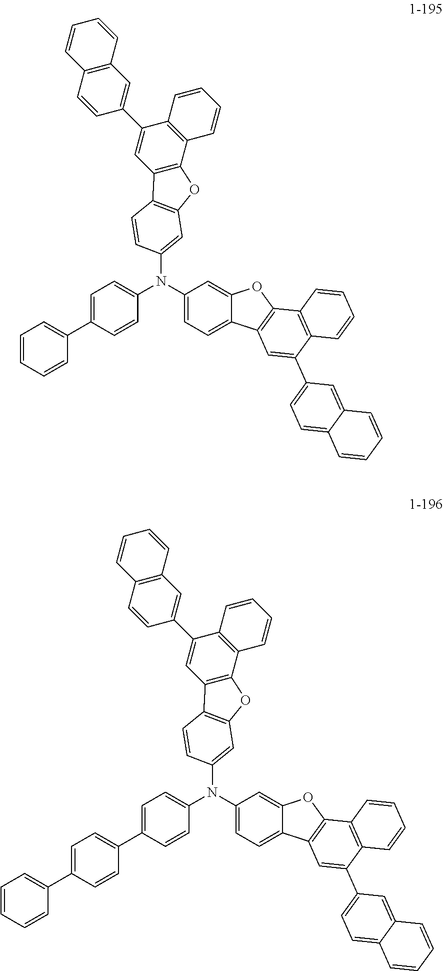 [SMILES: c1ccc(-c2ccc(N(c3ccc4c(c3)oc3c5ccccc5c(-c5ccc6ccccc6c5)cc43)c3ccc4c(c3)oc3c5ccccc5c(-c5ccc6ccccc6c5)cc43)cc2)cc1 ; c1ccc(-c2ccc(-c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc3)cc2)cc1]

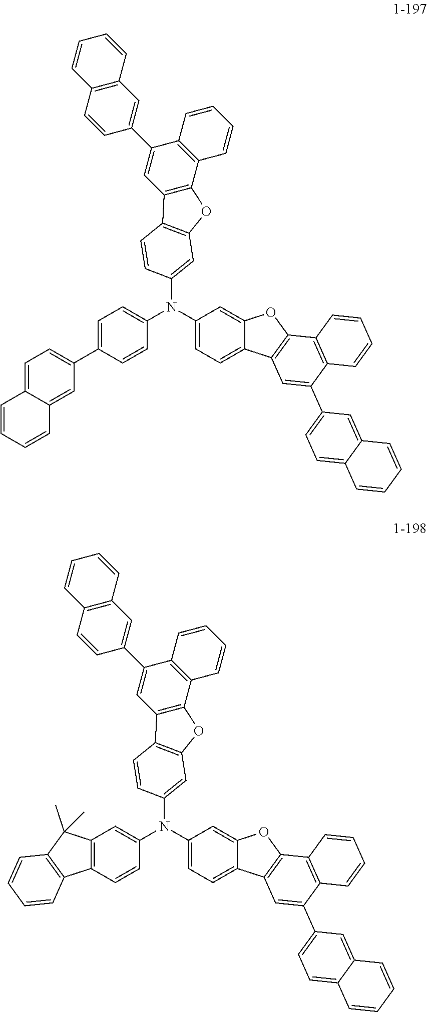 [SMILES: c1ccc2cc(-c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc3)ccc2c1 ; CC1(C)c2ccccc2-c2ccc(N(c3ccc4c(c3)oc3c5ccccc5c(-c5ccc6ccccc6c5)cc43)c3ccc4c(c3)oc3c5ccccc5c(-c5ccc6ccccc6c5)cc43)cc21]

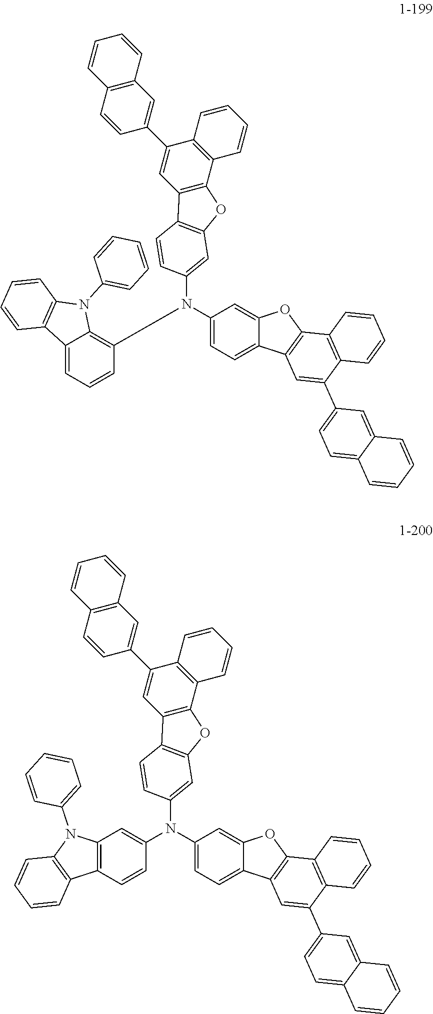 [SMILES: c1ccc(-n2c3ccccc3c3cccc(N(c4ccc5c(c4)oc4c6ccccc6c(-c6ccc7ccccc7c6)cc54)c4ccc5c(c4)oc4c6ccccc6c(-c6ccc7ccccc7c6)cc54)c32)cc1 ; c1ccc(-n2c3ccccc3c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc32)cc1]

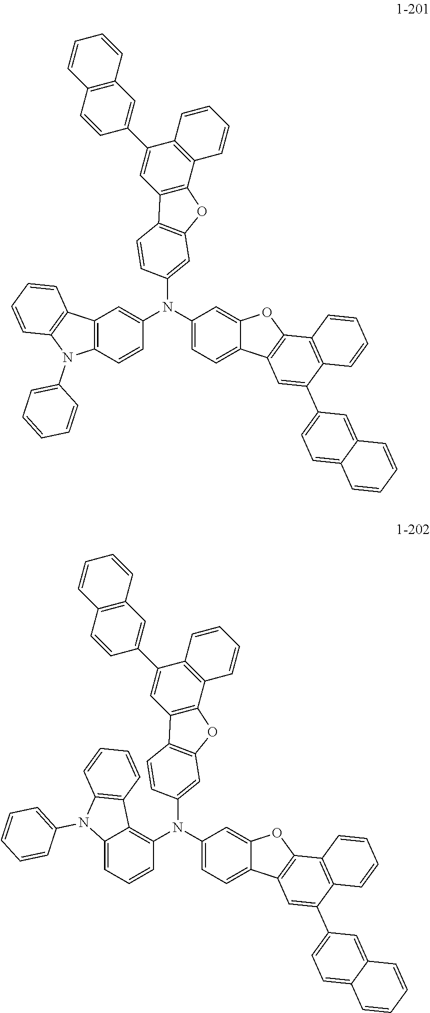 [SMILES: c1ccc(-n2c3ccccc3c3cc(N(c4ccc5c(c4)oc4c6ccccc6c(-c6ccc7ccccc7c6)cc54)c4ccc5c(c4)oc4c6ccccc6c(-c6ccc7ccccc7c6)cc54)ccc32)cc1 ; c1ccc(-n2c3ccccc3c3c(N(c4ccc5c(c4)oc4c6ccccc6c(-c6ccc7ccccc7c6)cc54)c4ccc5c(c4)oc4c6ccccc6c(-c6ccc7ccccc7c6)cc54)cccc32)cc1]

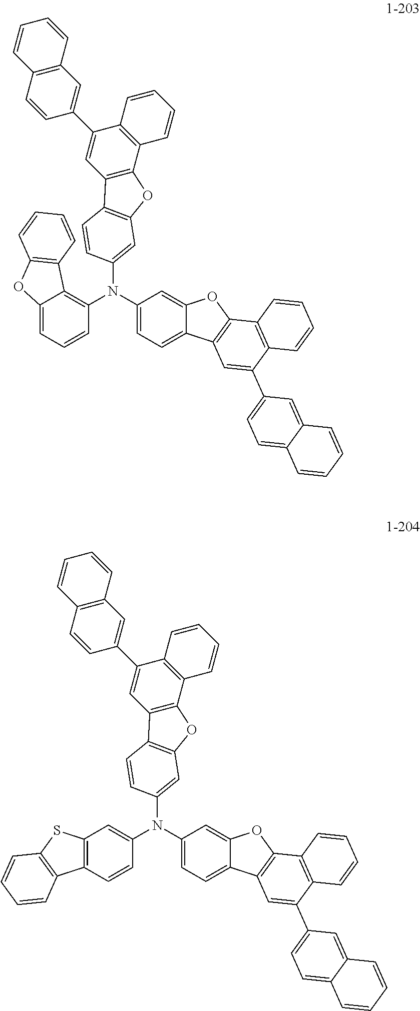 [SMILES: c1ccc2cc(-c3cc4c5ccc(N(c6ccc7c(c6)oc6c8ccccc8c(-c8ccc9ccccc9c8)cc76)c6cccc7oc8ccccc8c67)cc5oc4c4ccccc34)ccc2c1 ; c1ccc2cc(-c3cc4c5ccc(N(c6ccc7c(c6)oc6c8ccccc8c(-c8ccc9ccccc9c8)cc76)c6ccc7c(c6)sc6ccccc67)cc5oc4c4ccccc34)ccc2c1]

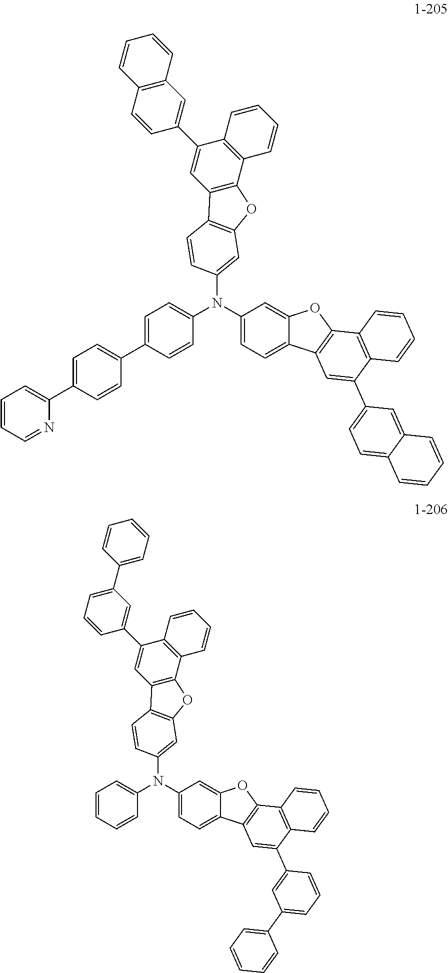 [SMILES: c1ccc(-c2ccc(-c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc3)cc2)nc1 ; c1ccc(-c2cccc(-c3cc4c5ccc(N(c6ccccc6)c6ccc7c(c6)oc6c8ccccc8c(-c8cccc(-c9ccccc9)c8)cc76)cc5oc4c4ccccc34)c2)cc1]

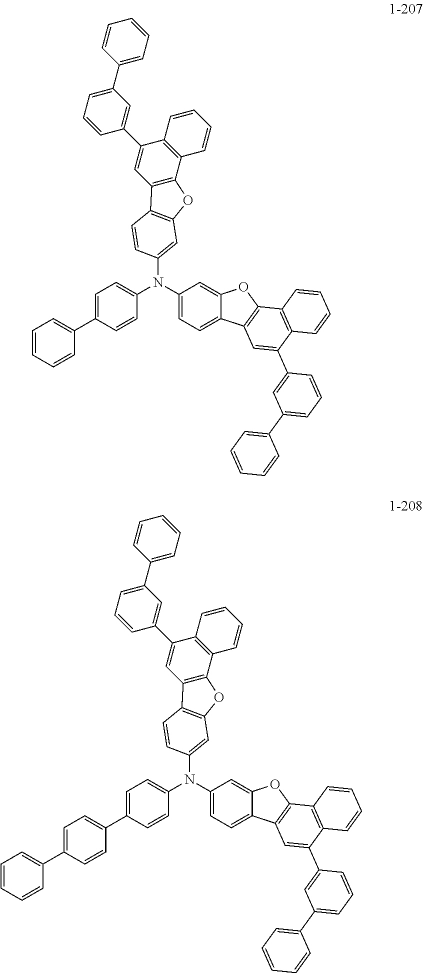 [SMILES: c1ccc(-c2ccc(N(c3ccc4c(c3)oc3c5ccccc5c(-c5cccc(-c6ccccc6)c5)cc43)c3ccc4c(c3)oc3c5ccccc5c(-c5cccc(-c6ccccc6)c5)cc43)cc2)cc1 ; c1ccc(-c2ccc(-c3ccc(N(c4ccc5c(c4)oc4c6ccccc6c(-c6cccc(-c7ccccc7)c6)cc54)c4ccc5c(c4)oc4c6ccccc6c(-c6cccc(-c7ccccc7)c6)cc54)cc3)cc2)cc1]

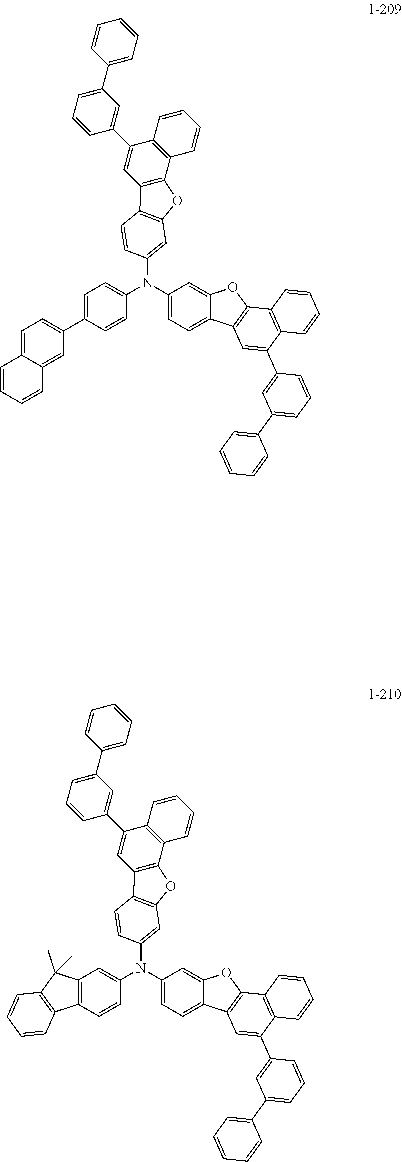 [SMILES: c1ccc(-c2cccc(-c3cc4c5ccc(N(c6ccc(-c7ccc8ccccc8c7)cc6)c6ccc7c(c6)oc6c8ccccc8c(-c8cccc(-c9ccccc9)c8)cc76)cc5oc4c4ccccc34)c2)cc1 ; CC1(C)c2ccccc2-c2ccc(N(c3ccc4c(c3)oc3c5ccccc5c(-c5cccc(-c6ccccc6)c5)cc43)c3ccc4c(c3)oc3c5ccccc5c(-c5cccc(-c6ccccc6)c5)cc43)cc21]

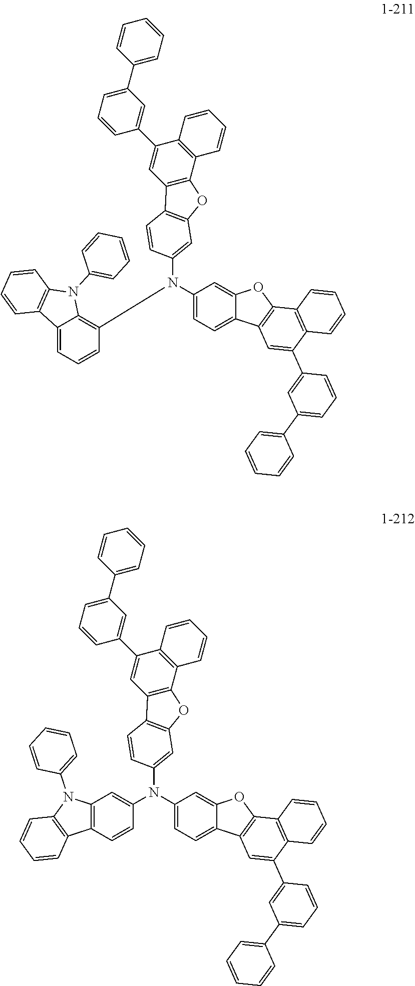 [SMILES: c1ccc(-c2cccc(-c3cc4c5ccc(N(c6ccc7c(c6)oc6c8ccccc8c(-c8cccc(-c9ccccc9)c8)cc76)c6cccc7c8ccccc8n(-c8ccccc8)c67)cc5oc4c4ccccc34)c2)cc1 ; c1ccc(-c2cccc(-c3cc4c5ccc(N(c6ccc7c(c6)oc6c8ccccc8c(-c8cccc(-c9ccccc9)c8)cc76)c6ccc7c8ccccc8n(-c8ccccc8)c7c6)cc5oc4c4ccccc34)c2)cc1]

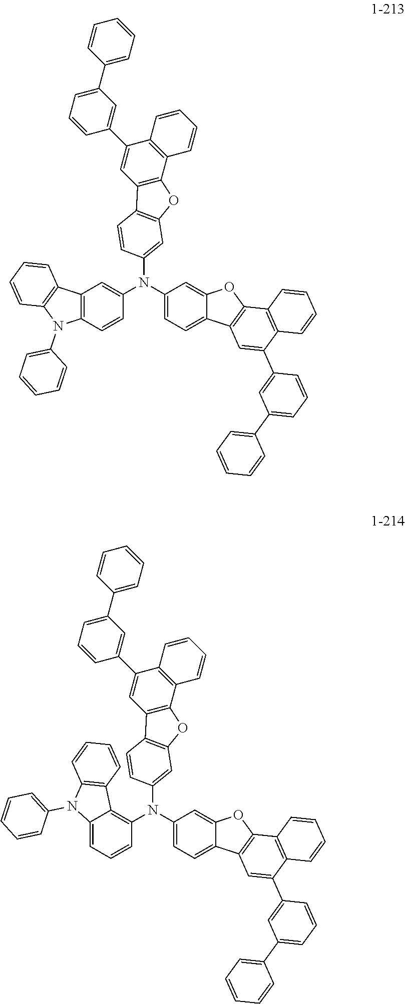 [SMILES: c1ccc(-c2cccc(-c3cc4c5ccc(N(c6ccc7c(c6)oc6c8ccccc8c(-c8cccc(-c9ccccc9)c8)cc76)c6ccc7c(c6)c6ccccc6n7-c6ccccc6)cc5oc4c4ccccc34)c2)cc1 ; c1ccc(-c2cccc(-c3cc4c5ccc(N(c6ccc7c(c6)oc6c8ccccc8c(-c8cccc(-c9ccccc9)c8)cc76)c6cccc7c6c6ccccc6n7-c6ccccc6)cc5oc4c4ccccc34)c2)cc1]

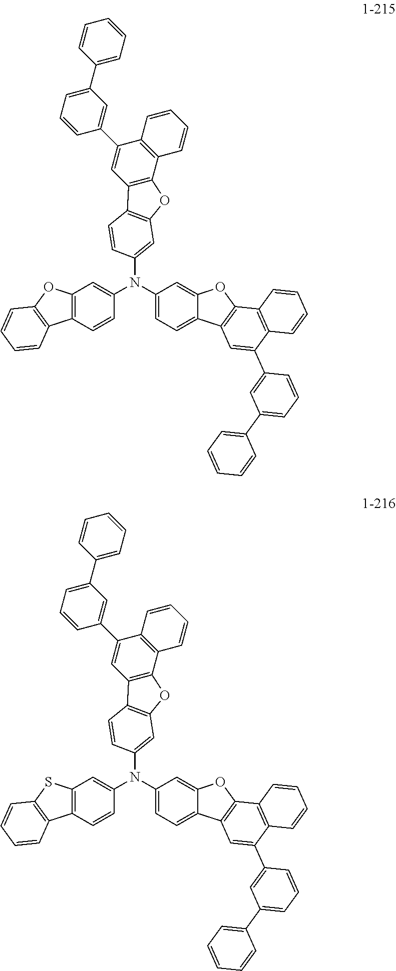 [SMILES: c1ccc(-c2cccc(-c3cc4c5ccc(N(c6ccc7c(c6)oc6ccccc67)c6ccc7c(c6)oc6c8ccccc8c(-c8cccc(-c9ccccc9)c8)cc76)cc5oc4c4ccccc34)c2)cc1 ; c1ccc(-c2cccc(-c3cc4c5ccc(N(c6ccc7c(c6)oc6c8ccccc8c(-c8cccc(-c9ccccc9)c8)cc76)c6ccc7c(c6)sc6ccccc67)cc5oc4c4ccccc34)c2)cc1]

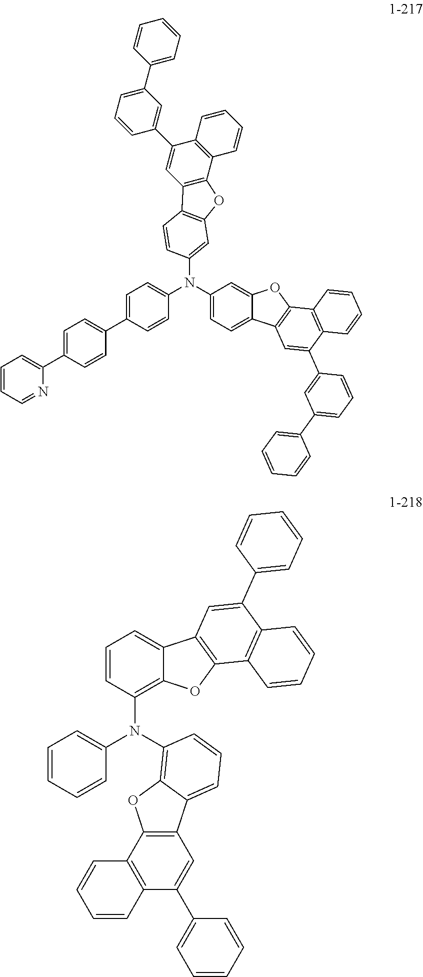 [SMILES: c1ccc(-c2cccc(-c3cc4c5ccc(N(c6ccc(-c7ccc(-c8ccccn8)cc7)cc6)c6ccc7c(c6)oc6c8ccccc8c(-c8cccc(-c9ccccc9)c8)cc76)cc5oc4c4ccccc34)c2)cc1 ; c1ccc(-c2cc3c4cccc(N(c5ccccc5)c5cccc6c5oc5c7ccccc7c(-c7ccccc7)cc65)c4oc3c3ccccc23)cc1]

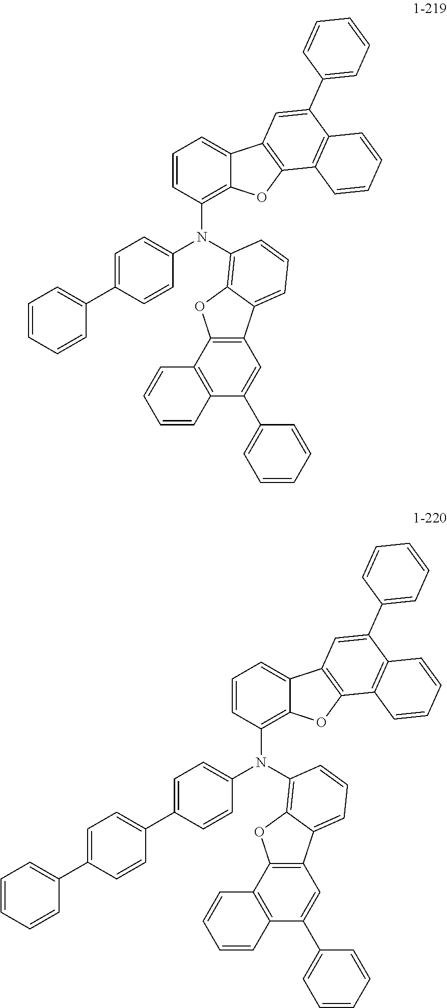 [SMILES: c1ccc(-c2ccc(N(c3cccc4c3oc3c5ccccc5c(-c5ccccc5)cc43)c3cccc4c3oc3c5ccccc5c(-c5ccccc5)cc43)cc2)cc1 ; c1ccc(-c2ccc(-c3ccc(N(c4cccc5c4oc4c6ccccc6c(-c6ccccc6)cc54)c4cccc5c4oc4c6ccccc6c(-c6ccccc6)cc54)cc3)cc2)cc1]

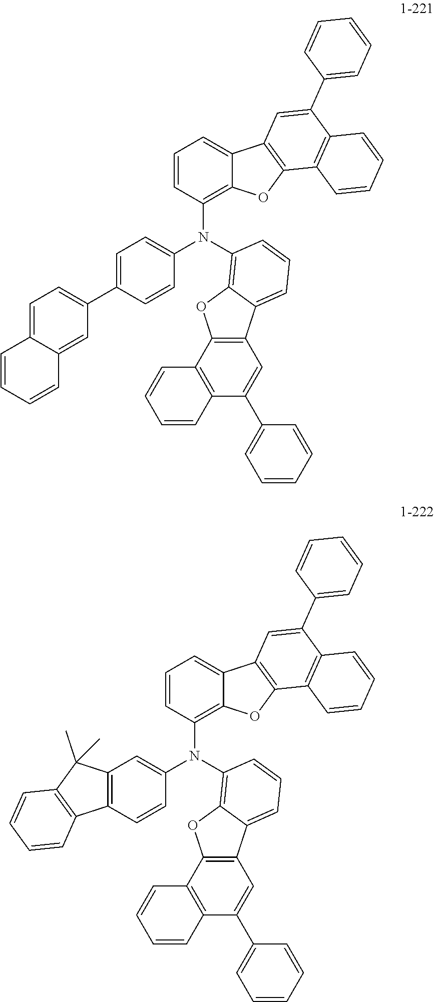 [SMILES: c1ccc(-c2cc3c4cccc(N(c5ccc(-c6ccc7ccccc7c6)cc5)c5cccc6c5oc5c7ccccc7c(-c7ccccc7)cc65)c4oc3c3ccccc23)cc1 ; CC1(C)c2ccccc2-c2ccc(N(c3cccc4c3oc3c5ccccc5c(-c5ccccc5)cc43)c3cccc4c3oc3c5ccccc5c(-c5ccccc5)cc43)cc21]

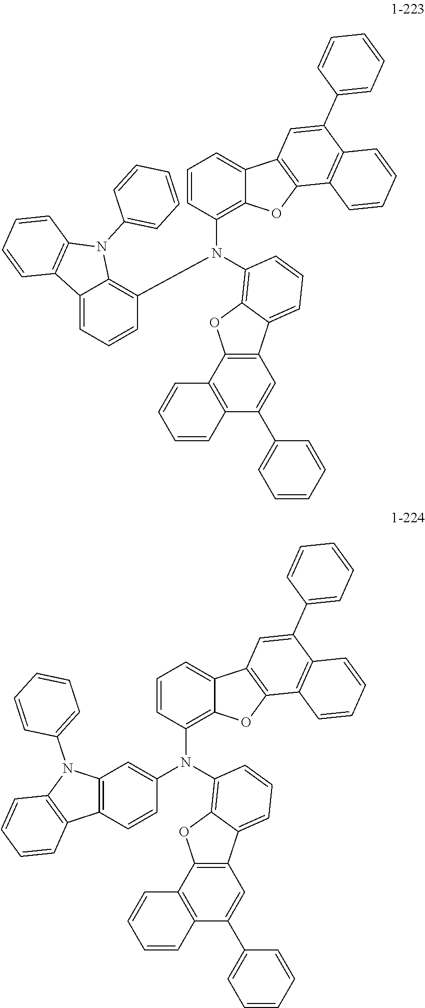 [SMILES: c1ccc(-c2cc3c4cccc(N(c5cccc6c5oc5c7ccccc7c(-c7ccccc7)cc65)c5cccc6c7ccccc7n(-c7ccccc7)c56)c4oc3c3ccccc23)cc1 ; c1ccc(-c2cc3c4cccc(N(c5ccc6c7ccccc7n(-c7ccccc7)c6c5)c5cccc6c5oc5c7ccccc7c(-c7ccccc7)cc65)c4oc3c3ccccc23)cc1]

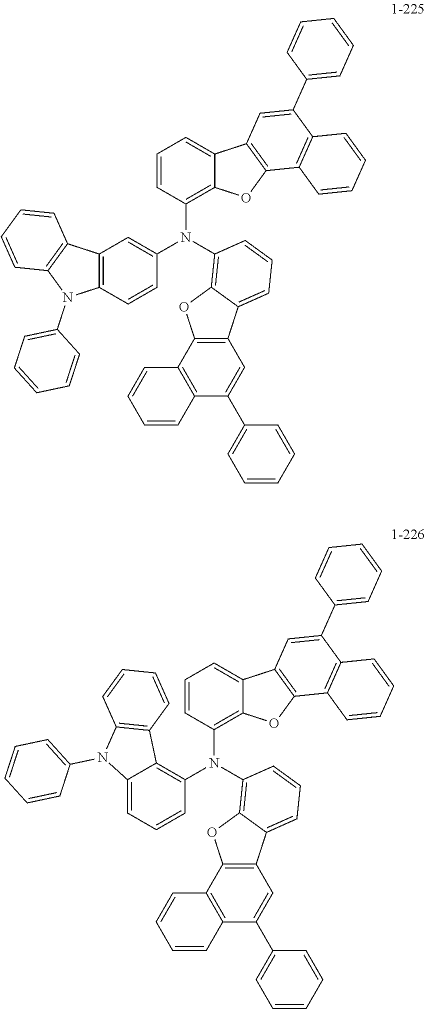 [SMILES: c1ccc(-c2cc3c4cccc(N(c5ccc6c(c5)c5ccccc5n6-c5ccccc5)c5cccc6c5oc5c7ccccc7c(-c7ccccc7)cc65)c4oc3c3ccccc23)cc1 ; c1ccc(-c2cc3c4cccc(N(c5cccc6c5oc5c7ccccc7c(-c7ccccc7)cc65)c5cccc6c5c5ccccc5n6-c5ccccc5)c4oc3c3ccccc23)cc1]

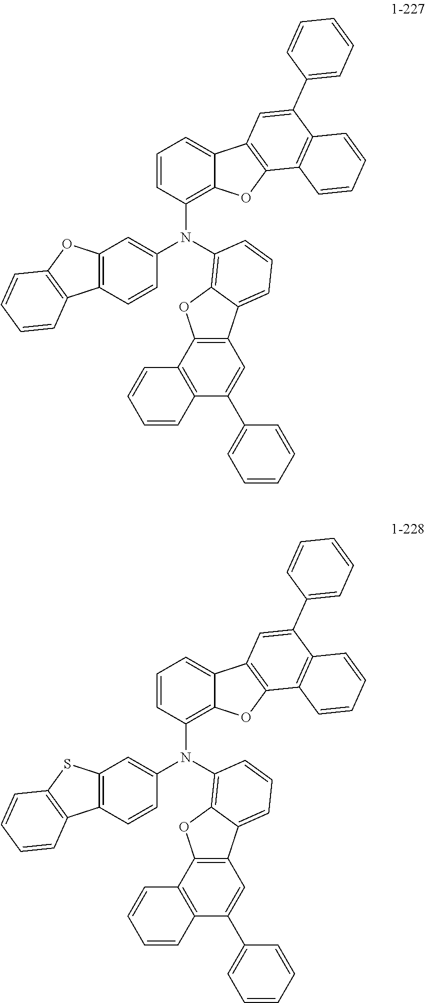 [SMILES: c1ccc(-c2cc3c4cccc(N(c5ccc6c(c5)oc5ccccc56)c5cccc6c5oc5c7ccccc7c(-c7ccccc7)cc65)c4oc3c3ccccc23)cc1 ; c1ccc(-c2cc3c4cccc(N(c5ccc6c(c5)sc5ccccc56)c5cccc6c5oc5c7ccccc7c(-c7ccccc7)cc65)c4oc3c3ccccc23)cc1]

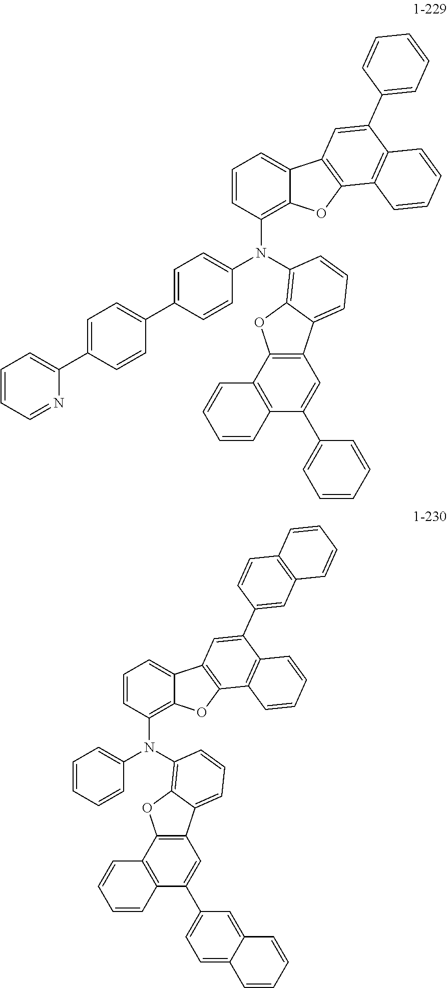

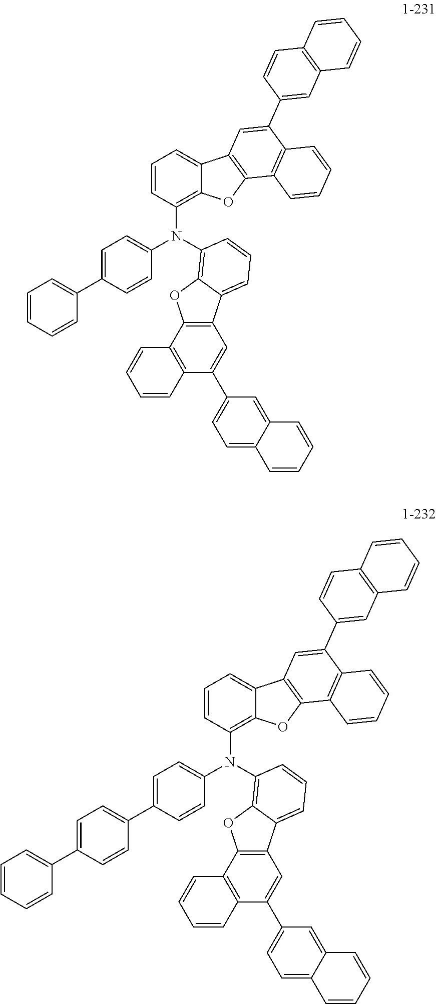 [SMILES: c1ccc(-c2ccc(N(c3cccc4c3oc3c5ccccc5c(-c5ccc6ccccc6c5)cc43)c3cccc4c3oc3c5ccccc5c(-c5ccc6ccccc6c5)cc43)cc2)cc1 ; c1ccc(-c2ccc(-c3ccc(N(c4cccc5c4oc4c6ccccc6c(-c6ccc7ccccc7c6)cc54)c4cccc5c4oc4c6ccccc6c(-c6ccc7ccccc7c6)cc54)cc3)cc2)cc1]

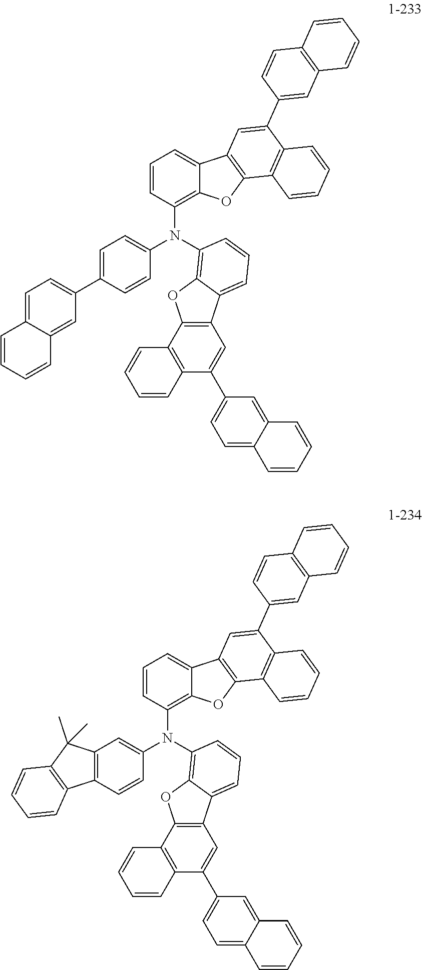 [SMILES: c1ccc2cc(-c3ccc(N(c4cccc5c4oc4c6ccccc6c(-c6ccc7ccccc7c6)cc54)c4cccc5c4oc4c6ccccc6c(-c6ccc7ccccc7c6)cc54)cc3)ccc2c1 ; CC1(C)c2ccccc2-c2ccc(N(c3cccc4c3oc3c5ccccc5c(-c5ccc6ccccc6c5)cc43)c3cccc4c3oc3c5ccccc5c(-c5ccc6ccccc6c5)cc43)cc21]

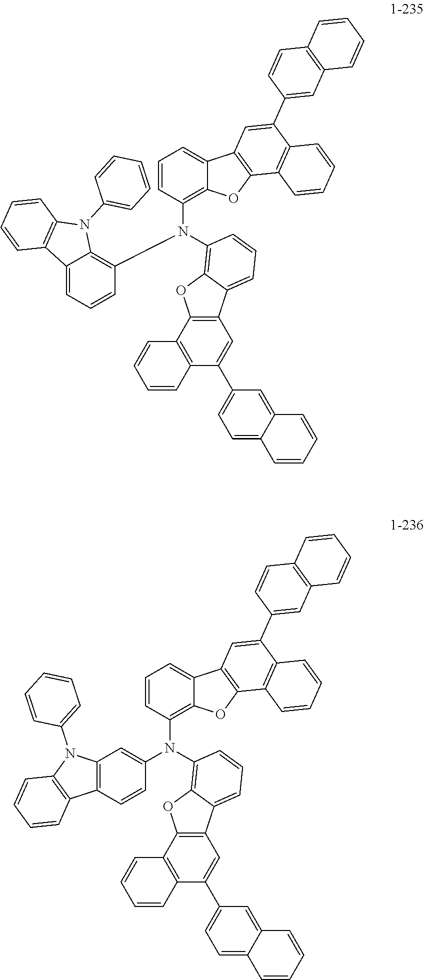 [SMILES: c1ccc(-n2c3ccccc3c3cccc(N(c4cccc5c4oc4c6ccccc6c(-c6ccc7ccccc7c6)cc54)c4cccc5c4oc4c6ccccc6c(-c6ccc7ccccc7c6)cc54)c32)cc1 ; c1ccc(-n2c3ccccc3c3ccc(N(c4cccc5c4oc4c6ccccc6c(-c6ccc7ccccc7c6)cc54)c4cccc5c4oc4c6ccccc6c(-c6ccc7ccccc7c6)cc54)cc32)cc1]

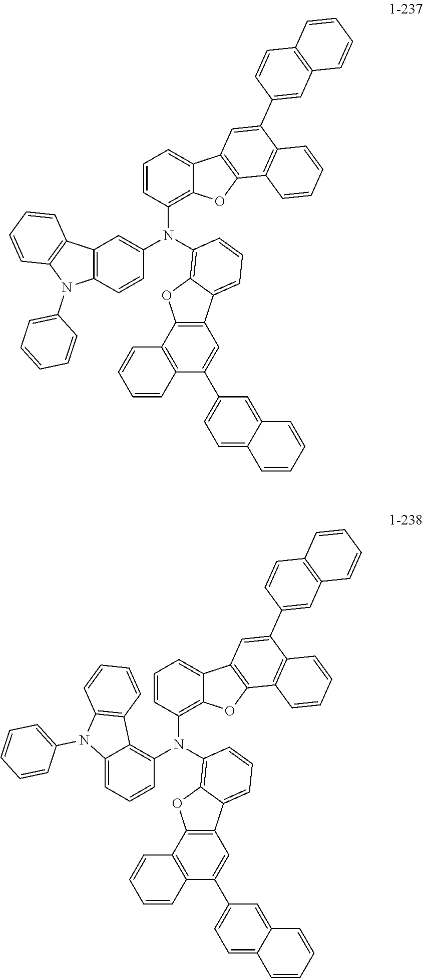 [SMILES: c1ccc(-n2c3ccccc3c3cc(N(c4cccc5c4oc4c6ccccc6c(-c6ccc7ccccc7c6)cc54)c4cccc5c4oc4c6ccccc6c(-c6ccc7ccccc7c6)cc54)ccc32)cc1 ; c1ccc(-n2c3ccccc3c3c(N(c4cccc5c4oc4c6ccccc6c(-c6ccc7ccccc7c6)cc54)c4cccc5c4oc4c6ccccc6c(-c6ccc7ccccc7c6)cc54)cccc32)cc1]

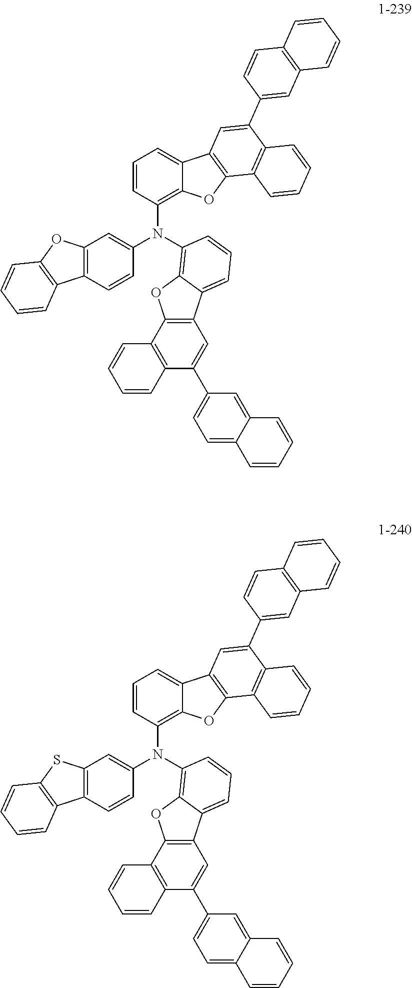 [SMILES: c1ccc2cc(-c3cc4c5cccc(N(c6ccc7c(c6)oc6ccccc67)c6cccc7c6oc6c8ccccc8c(-c8ccc9ccccc9c8)cc76)c5oc4c4ccccc34)ccc2c1 ; c1ccc2cc(-c3cc4c5cccc(N(c6ccc7c(c6)sc6ccccc67)c6cccc7c6oc6c8ccccc8c(-c8ccc9ccccc9c8)cc76)c5oc4c4ccccc34)ccc2c1]

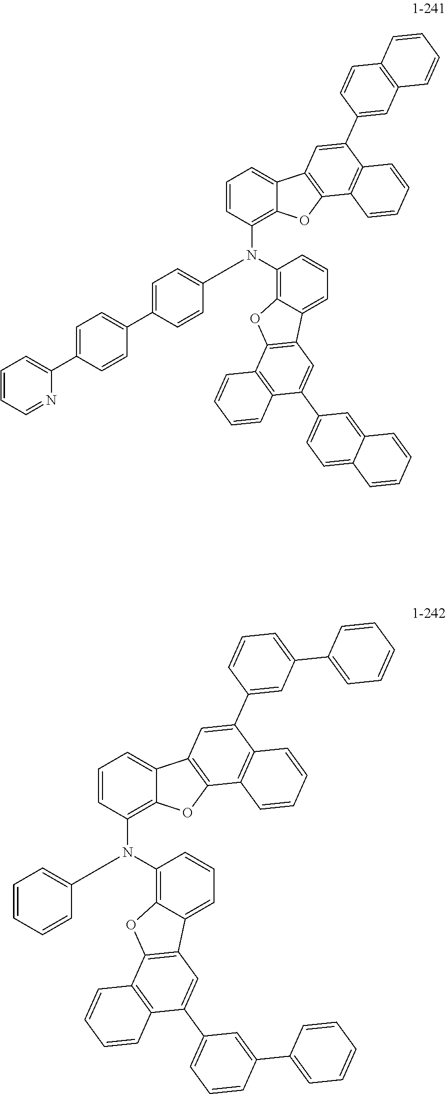 [SMILES: c1ccc(-c2ccc(-c3ccc(N(c4cccc5c4oc4c6ccccc6c(-c6ccc7ccccc7c6)cc54)c4cccc5c4oc4c6ccccc6c(-c6ccc7ccccc7c6)cc54)cc3)cc2)nc1 ; c1ccc(-c2cccc(-c3cc4c5cccc(N(c6ccccc6)c6cccc7c6oc6c8ccccc8c(-c8cccc(-c9ccccc9)c8)cc76)c5oc4c4ccccc34)c2)cc1]

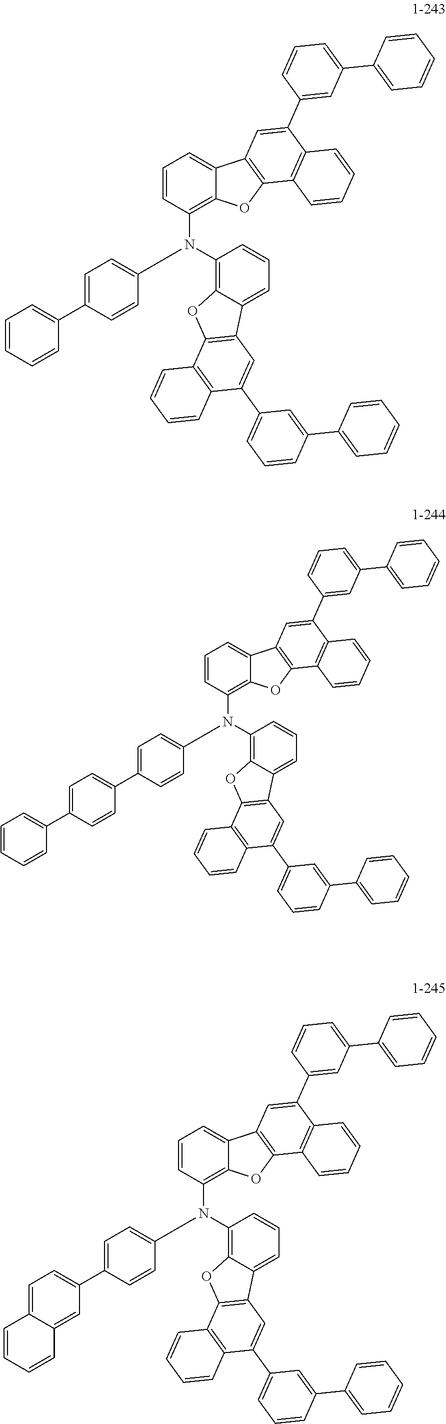 [SMILES: c1ccc(-c2ccc(N(c3cccc4c3oc3c5ccccc5c(-c5cccc(-c6ccccc6)c5)cc43)c3cccc4c3oc3c5ccccc5c(-c5cccc(-c6ccccc6)c5)cc43)cc2)cc1 ; c1ccc(-c2ccc(-c3ccc(N(c4cccc5c4oc4c6ccccc6c(-c6cccc(-c7ccccc7)c6)cc54)c4cccc5c4oc4c6ccccc6c(-c6cccc(-c7ccccc7)c6)cc54)cc3)cc2)cc1 ; c1ccc(-c2cccc(-c3cc4c5cccc(N(c6ccc(-c7ccc8ccccc8c7)cc6)c6cccc7c6oc6c8ccccc8c(-c8cccc(-c9ccccc9)c8)cc76)c5oc4c4ccccc34)c2)cc1]

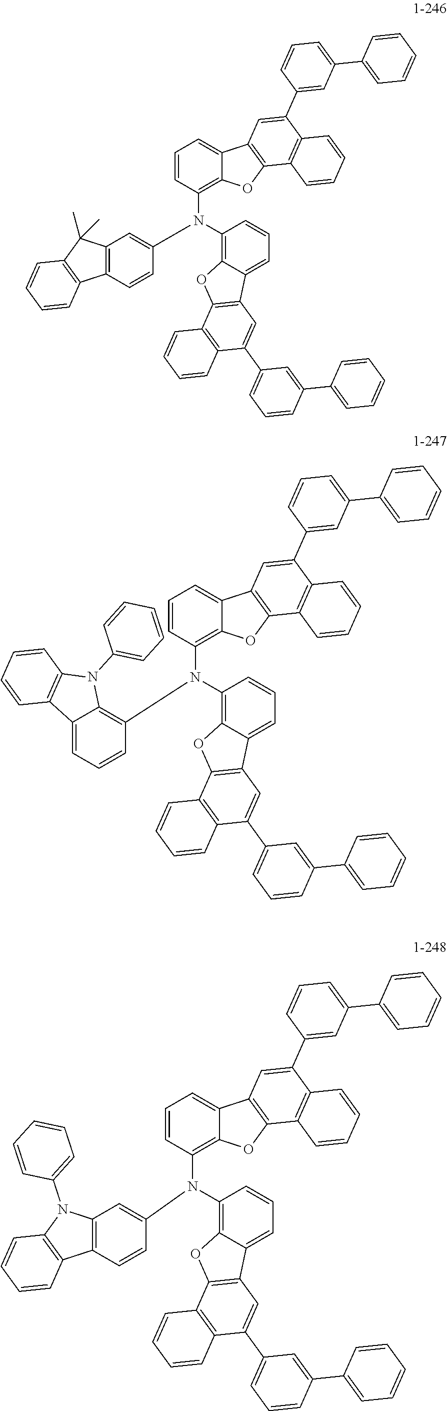 [SMILES: CC1(C)c2ccccc2-c2ccc(N(c3cccc4c3oc3c5ccccc5c(-c5cccc(-c6ccccc6)c5)cc43)c3cccc4c3oc3c5ccccc5c(-c5cccc(-c6ccccc6)c5)cc43)cc21 ; c1ccc(-c2cccc(-c3cc4c5cccc(N(c6cccc7c6oc6c8ccccc8c(-c8cccc(-c9ccccc9)c8)cc76)c6cccc7c8ccccc8n(-c8ccccc8)c67)c5oc4c4ccccc34)c2)cc1 ; c1ccc(-c2cccc(-c3cc4c5cccc(N(c6ccc7c8ccccc8n(-c8ccccc8)c7c6)c6cccc7c6oc6c8ccccc8c(-c8cccc(-c9ccccc9)c8)cc76)c5oc4c4ccccc34)c2)cc1]

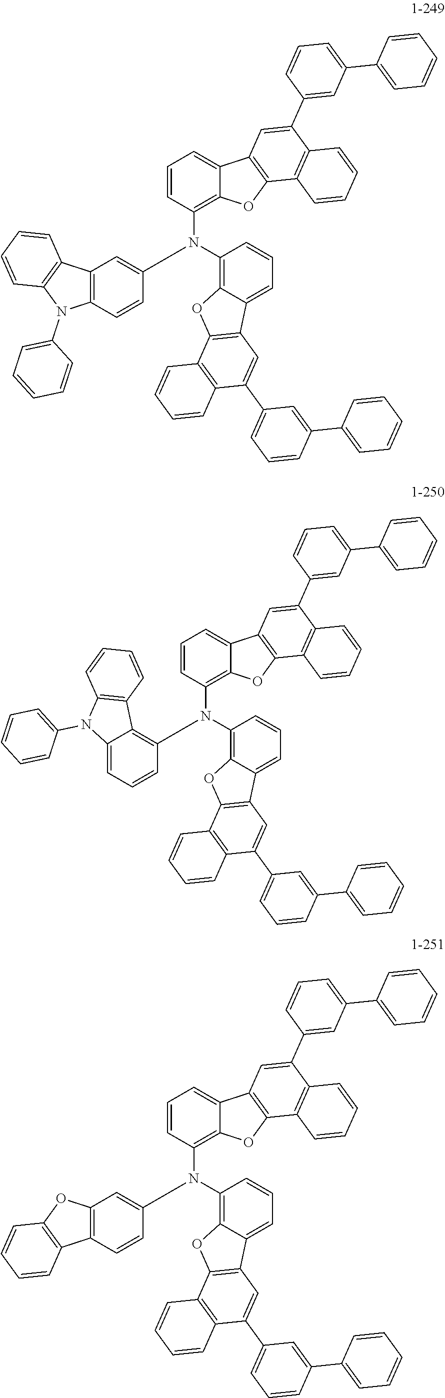 [SMILES: c1ccc(-c2cccc(-c3cc4c5cccc(N(c6ccc7c(c6)c6ccccc6n7-c6ccccc6)c6cccc7c6oc6c8ccccc8c(-c8cccc(-c9ccccc9)c8)cc76)c5oc4c4ccccc34)c2)cc1 ; c1ccc(-c2cccc(-c3cc4c5cccc(N(c6cccc7c6oc6c8ccccc8c(-c8cccc(-c9ccccc9)c8)cc76)c6cccc7c6c6ccccc6n7-c6ccccc6)c5oc4c4ccccc34)c2)cc1 ; c1ccc(-c2cccc(-c3cc4c5cccc(N(c6ccc7c(c6)oc6ccccc67)c6cccc7c6oc6c8ccccc8c(-c8cccc(-c9ccccc9)c8)cc76)c5oc4c4ccccc34)c2)cc1]

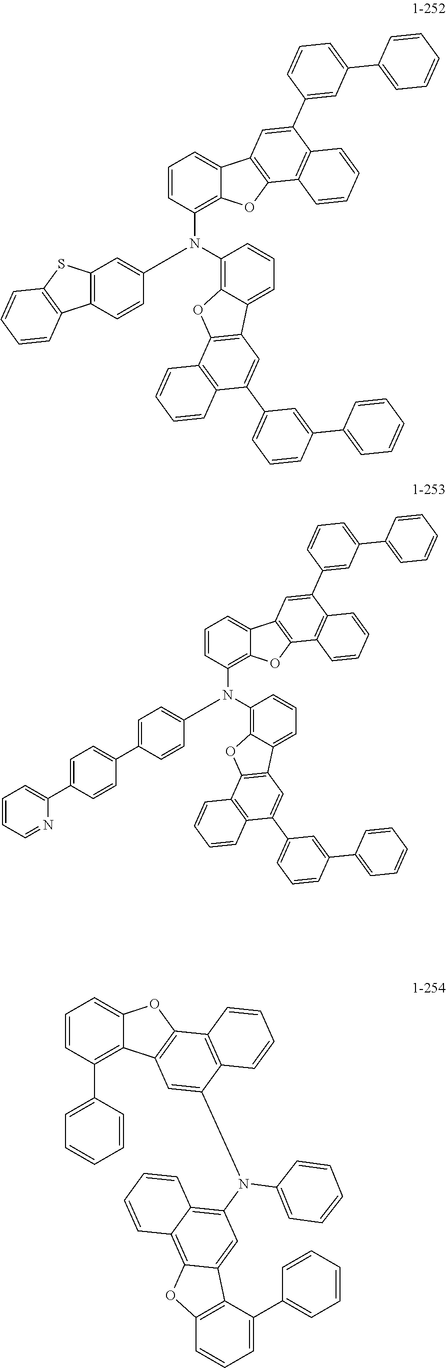 [SMILES: c1ccc(-c2cccc(-c3cc4c5cccc(N(c6ccc7c(c6)sc6ccccc67)c6cccc7c6oc6c8ccccc8c(-c8cccc(-c9ccccc9)c8)cc76)c5oc4c4ccccc34)c2)cc1 ; c1ccc(-c2cccc(-c3cc4c5cccc(N(c6ccc(-c7ccc(-c8ccccn8)cc7)cc6)c6cccc7c6oc6c8ccccc8c(-c8cccc(-c9ccccc9)c8)cc76)c5oc4c4ccccc34)c2)cc1 ; c1ccc(-c2cccc3oc4c5ccccc5c(N(c5ccccc5)c5cc6c(oc7cccc(-c8ccccc8)c76)c6ccccc56)cc4c23)cc1]

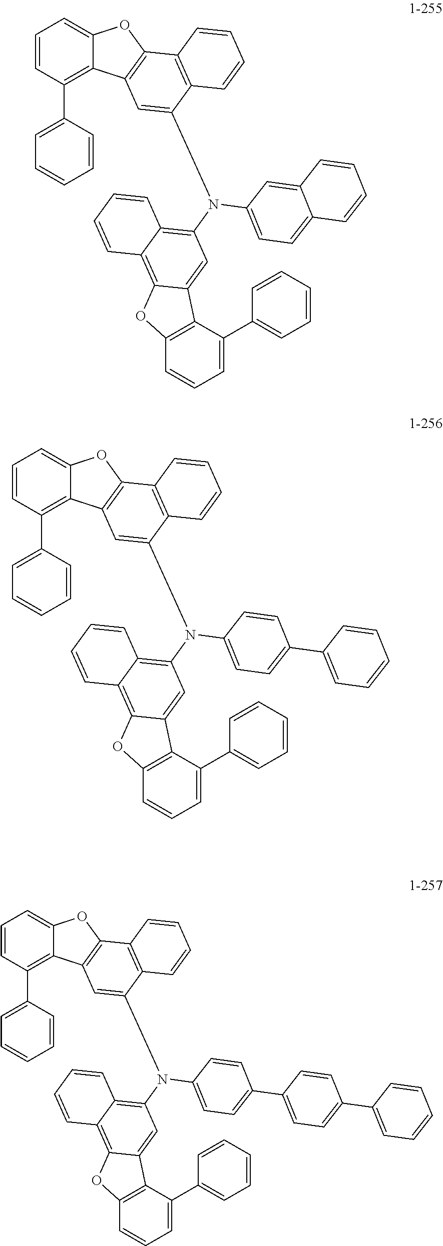 [SMILES: c1ccc(-c2cccc3oc4c5ccccc5c(N(c5ccc6ccccc6c5)c5cc6c(oc7cccc(-c8ccccc8)c76)c6ccccc56)cc4c23)cc1 ; c1ccc(-c2ccc(N(c3cc4c(oc5cccc(-c6ccccc6)c54)c4ccccc34)c3cc4c(oc5cccc(-c6ccccc6)c54)c4ccccc34)cc2)cc1 ; c1ccc(-c2ccc(-c3ccc(N(c4cc5c(oc6cccc(-c7ccccc7)c65)c5ccccc45)c4cc5c(oc6cccc(-c7ccccc7)c65)c5ccccc45)cc3)cc2)cc1]

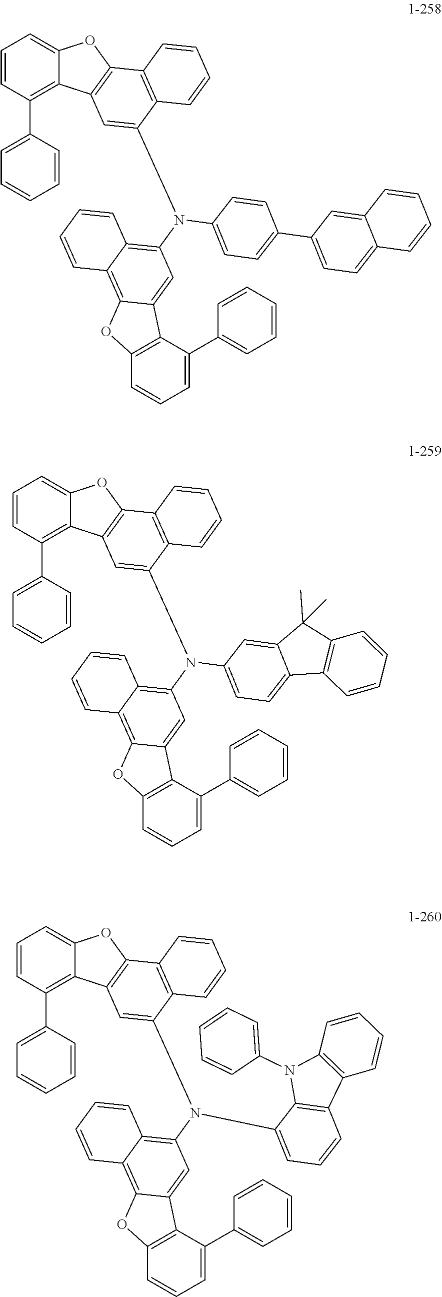 [SMILES: c1ccc(-c2cccc3oc4c5ccccc5c(N(c5ccc(-c6ccc7ccccc7c6)cc5)c5cc6c(oc7cccc(-c8ccccc8)c76)c6ccccc56)cc4c23)cc1 ; CC1(C)c2ccccc2-c2ccc(N(c3cc4c(oc5cccc(-c6ccccc6)c54)c4ccccc34)c3cc4c(oc5cccc(-c6ccccc6)c54)c4ccccc34)cc21 ; c1ccc(-c2cccc3oc4c5ccccc5c(N(c5cc6c(oc7cccc(-c8ccccc8)c76)c6ccccc56)c5cccc6c7ccccc7n(-c7ccccc7)c56)cc4c23)cc1]

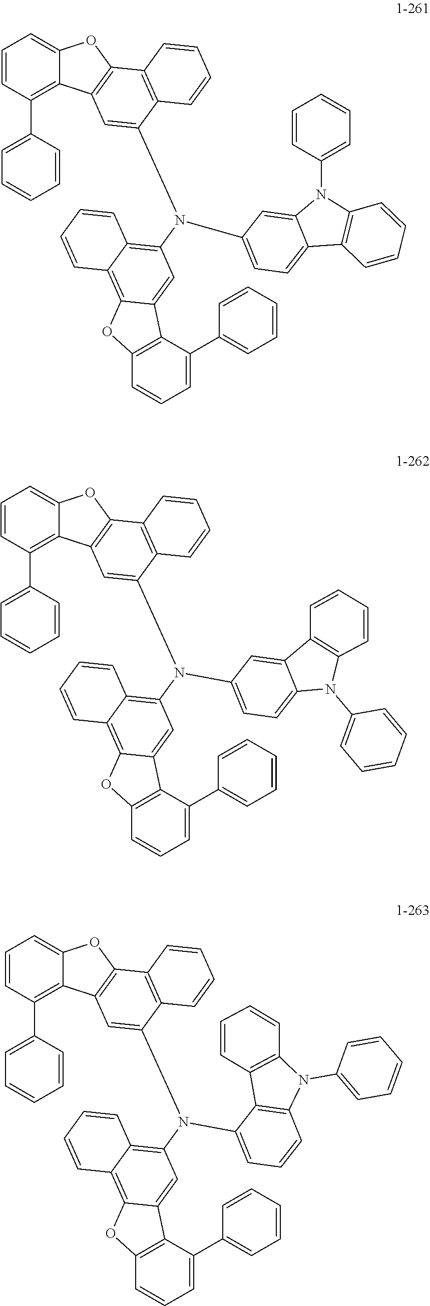 [SMILES: c1ccc(-c2cccc3oc4c5ccccc5c(N(c5ccc6c7ccccc7n(-c7ccccc7)c6c5)c5cc6c(oc7cccc(-c8ccccc8)c76)c6ccccc56)cc4c23)cc1 ; c1ccc(-c2cccc3oc4c5ccccc5c(N(c5ccc6c(c5)c5ccccc5n6-c5ccccc5)c5cc6c(oc7cccc(-c8ccccc8)c76)c6ccccc56)cc4c23)cc1 ; c1ccc(-c2cccc3oc4c5ccccc5c(N(c5cc6c(oc7cccc(-c8ccccc8)c76)c6ccccc56)c5cccc6c5c5ccccc5n6-c5ccccc5)cc4c23)cc1]

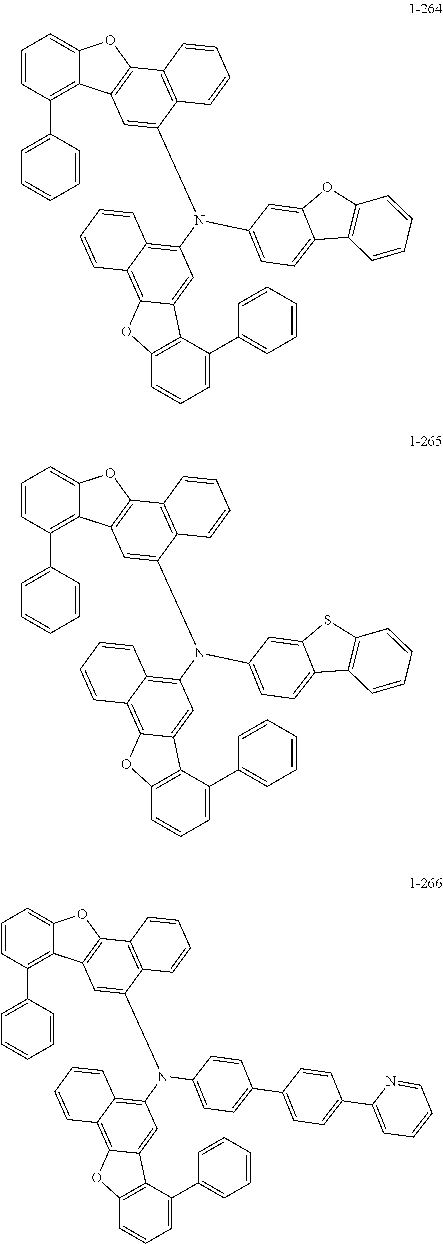 [SMILES: c1ccc(-c2cccc3oc4c5ccccc5c(N(c5ccc6c(c5)oc5ccccc56)c5cc6c(oc7cccc(-c8ccccc8)c76)c6ccccc56)cc4c23)cc1 ; c1ccc(-c2cccc3oc4c5ccccc5c(N(c5ccc6c(c5)sc5ccccc56)c5cc6c(oc7cccc(-c8ccccc8)c76)c6ccccc56)cc4c23)cc1 ; c1ccc(-c2cccc3oc4c5ccccc5c(N(c5ccc(-c6ccc(-c7ccccn7)cc6)cc5)c5cc6c(oc7cccc(-c8ccccc8)c76)c6ccccc56)cc4c23)cc1]

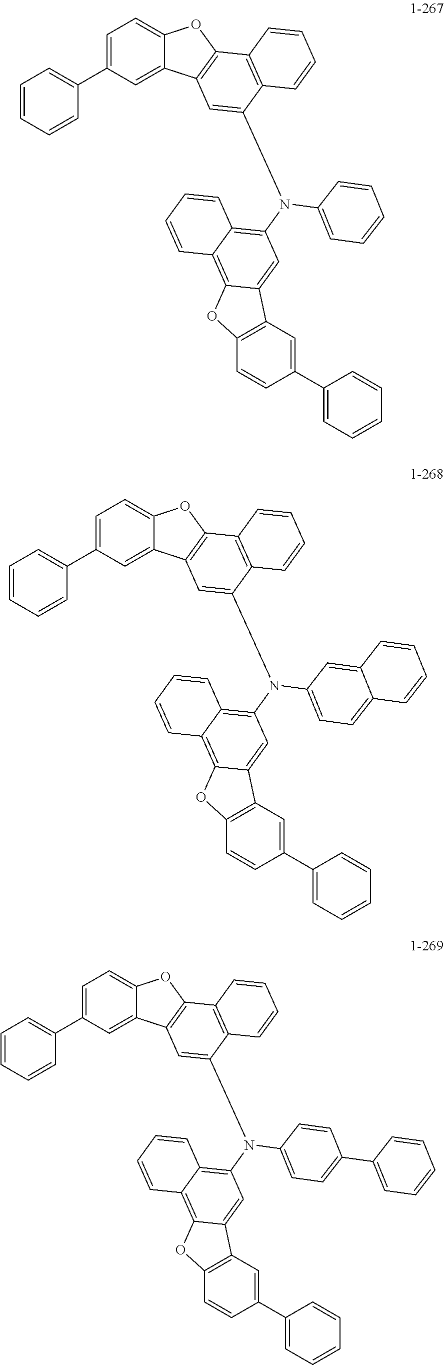 [SMILES: c1ccc(-c2ccc3oc4c5ccccc5c(N(c5ccccc5)c5cc6c7cc(-c8ccccc8)ccc7oc6c6ccccc56)cc4c3c2)cc1 ; c1ccc(-c2ccc3oc4c5ccccc5c(N(c5ccc6ccccc6c5)c5cc6c7cc(-c8ccccc8)ccc7oc6c6ccccc56)cc4c3c2)cc1 ; c1ccc(-c2ccc(N(c3cc4c5cc(-c6ccccc6)ccc5oc4c4ccccc34)c3cc4c5cc(-c6ccccc6)ccc5oc4c4ccccc34)cc2)cc1]

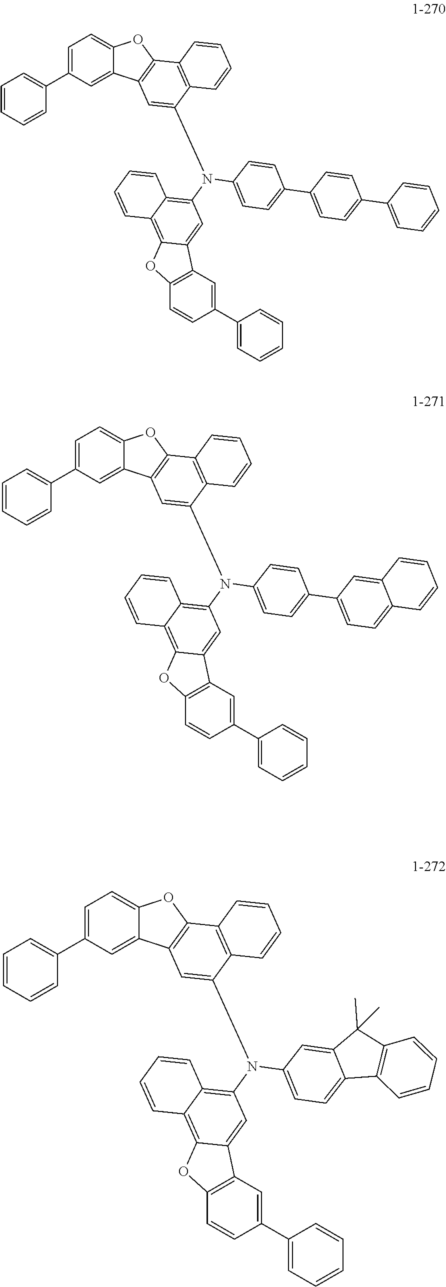 [SMILES: c1ccc(-c2ccc(-c3ccc(N(c4cc5c6cc(-c7ccccc7)ccc6oc5c5ccccc45)c4cc5c6cc(-c7ccccc7)ccc6oc5c5ccccc45)cc3)cc2)cc1 ; c1ccc(-c2ccc3oc4c5ccccc5c(N(c5ccc(-c6ccc7ccccc7c6)cc5)c5cc6c7cc(-c8ccccc8)ccc7oc6c6ccccc56)cc4c3c2)cc1 ; CC1(C)c2ccccc2-c2ccc(N(c3cc4c5cc(-c6ccccc6)ccc5oc4c4ccccc34)c3cc4c5cc(-c6ccccc6)ccc5oc4c4ccccc34)cc21]

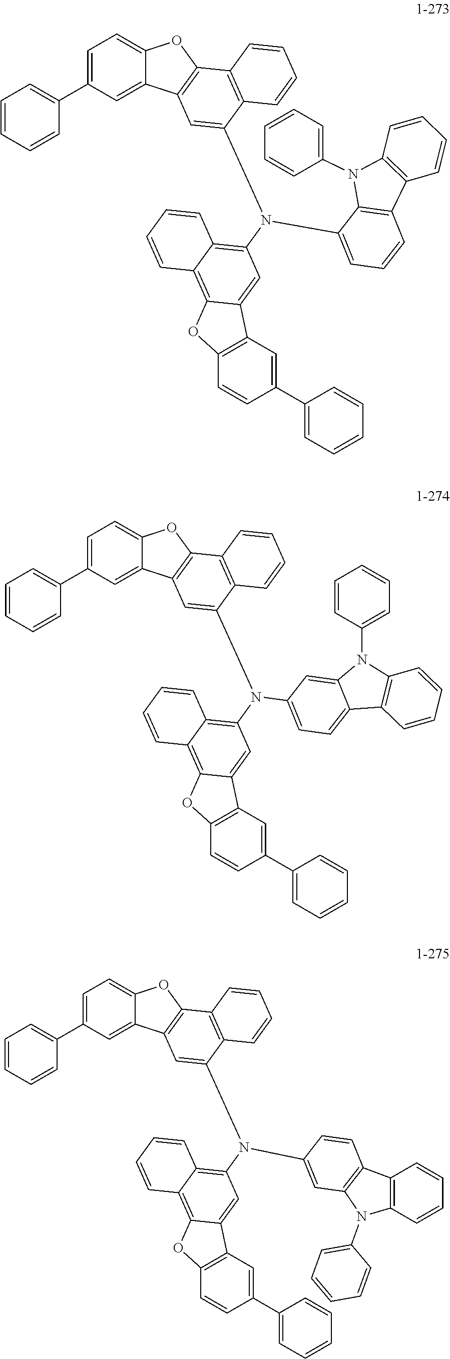 [SMILES: c1ccc(-c2ccc3oc4c5ccccc5c(N(c5cc6c7cc(-c8ccccc8)ccc7oc6c6ccccc56)c5cccc6c7ccccc7n(-c7ccccc7)c56)cc4c3c2)cc1 ; c1ccc(-c2ccc3oc4c5ccccc5c(N(c5ccc6c7ccccc7n(-c7ccccc7)c6c5)c5cc6c7cc(-c8ccccc8)ccc7oc6c6ccccc56)cc4c3c2)cc1 ; c1ccc(-c2ccc3oc4c5ccccc5c(N(c5ccc6c7ccccc7n(-c7ccccc7)c6c5)c5cc6c7cc(-c8ccccc8)ccc7oc6c6ccccc56)cc4c3c2)cc1]

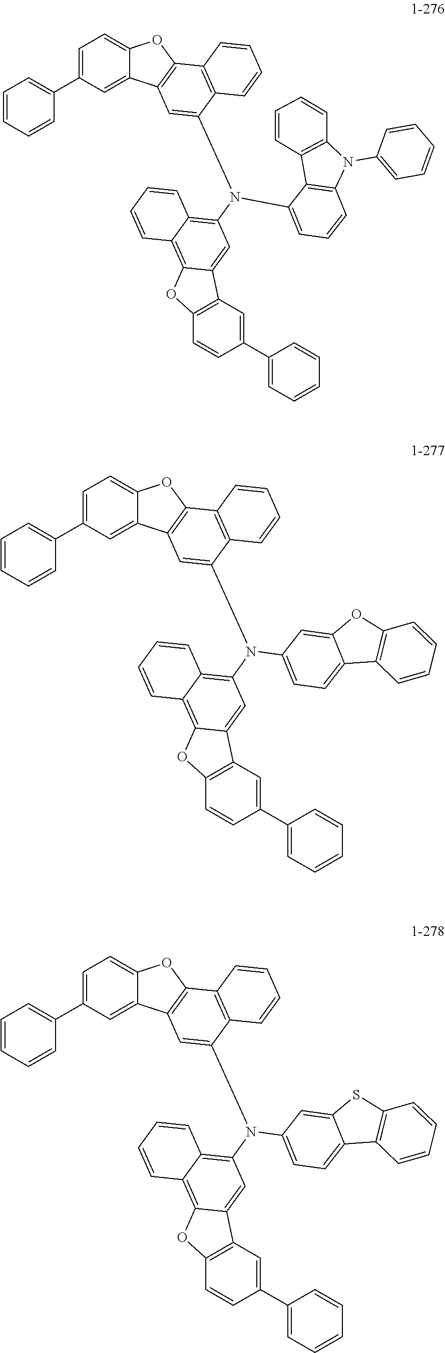 [SMILES: c1ccc(-c2ccc3oc4c5ccccc5c(N(c5cc6c7cc(-c8ccccc8)ccc7oc6c6ccccc56)c5cccc6c5c5ccccc5n6-c5ccccc5)cc4c3c2)cc1 ; c1ccc(-c2ccc3oc4c5ccccc5c(N(c5ccc6c(c5)oc5ccccc56)c5cc6c7cc(-c8ccccc8)ccc7oc6c6ccccc56)cc4c3c2)cc1 ; c1ccc(-c2ccc3oc4c5ccccc5c(N(c5ccc6c(c5)sc5ccccc56)c5cc6c7cc(-c8ccccc8)ccc7oc6c6ccccc56)cc4c3c2)cc1]

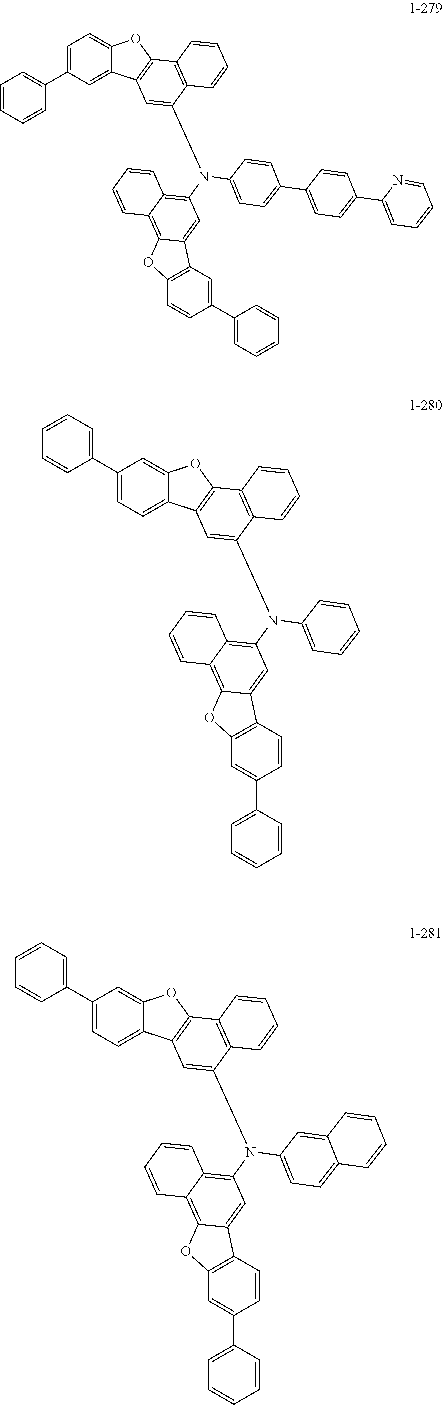 [SMILES: c1ccc(-c2ccc3oc4c5ccccc5c(N(c5ccc(-c6ccc(-c7ccccn7)cc6)cc5)c5cc6c7cc(-c8ccccc8)ccc7oc6c6ccccc56)cc4c3c2)cc1 ; c1ccc(-c2ccc3c(c2)oc2c4ccccc4c(N(c4ccccc4)c4cc5c6ccc(-c7ccccc7)cc6oc5c5ccccc45)cc32)cc1 ; c1ccc(-c2ccc3c(c2)oc2c4ccccc4c(N(c4ccc5ccccc5c4)c4cc5c6ccc(-c7ccccc7)cc6oc5c5ccccc45)cc32)cc1]

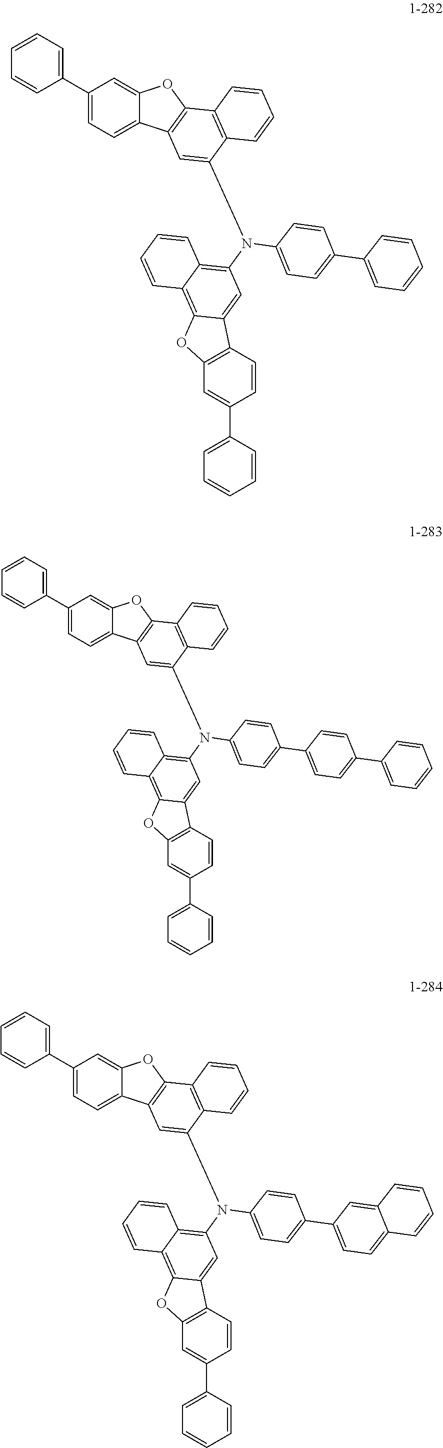 [SMILES: c1ccc(-c2ccc(N(c3cc4c5ccc(-c6ccccc6)cc5oc4c4ccccc34)c3cc4c5ccc(-c6ccccc6)cc5oc4c4ccccc34)cc2)cc1 ; c1ccc(-c2ccc(-c3ccc(N(c4cc5c6ccc(-c7ccccc7)cc6oc5c5ccccc45)c4cc5c6ccc(-c7ccccc7)cc6oc5c5ccccc45)cc3)cc2)cc1 ; c1ccc(-c2ccc3c(c2)oc2c4ccccc4c(N(c4ccc(-c5ccc6ccccc6c5)cc4)c4cc5c6ccc(-c7ccccc7)cc6oc5c5ccccc45)cc32)cc1]

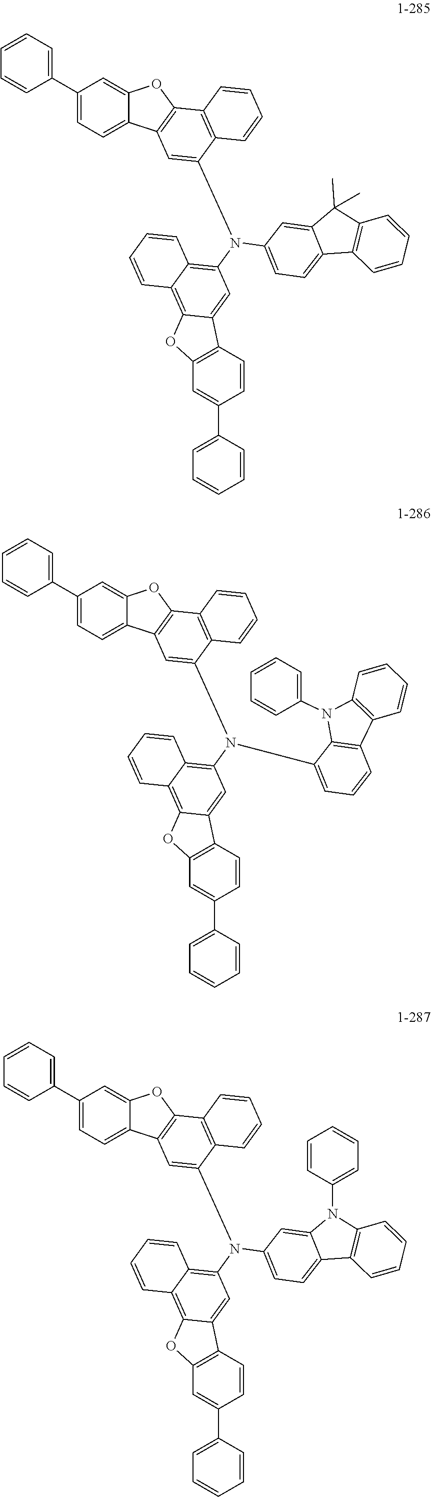 [SMILES: CC1(C)c2ccccc2-c2ccc(N(c3cc4c5ccc(-c6ccccc6)cc5oc4c4ccccc34)c3cc4c5ccc(-c6ccccc6)cc5oc4c4ccccc34)cc21 ; c1ccc(-c2ccc3c(c2)oc2c4ccccc4c(N(c4cc5c6ccc(-c7ccccc7)cc6oc5c5ccccc45)c4cccc5c6ccccc6n(-c6ccccc6)c45)cc32)cc1 ; c1ccc(-c2ccc3c(c2)oc2c4ccccc4c(N(c4ccc5c6ccccc6n(-c6ccccc6)c5c4)c4cc5c6ccc(-c7ccccc7)cc6oc5c5ccccc45)cc32)cc1]

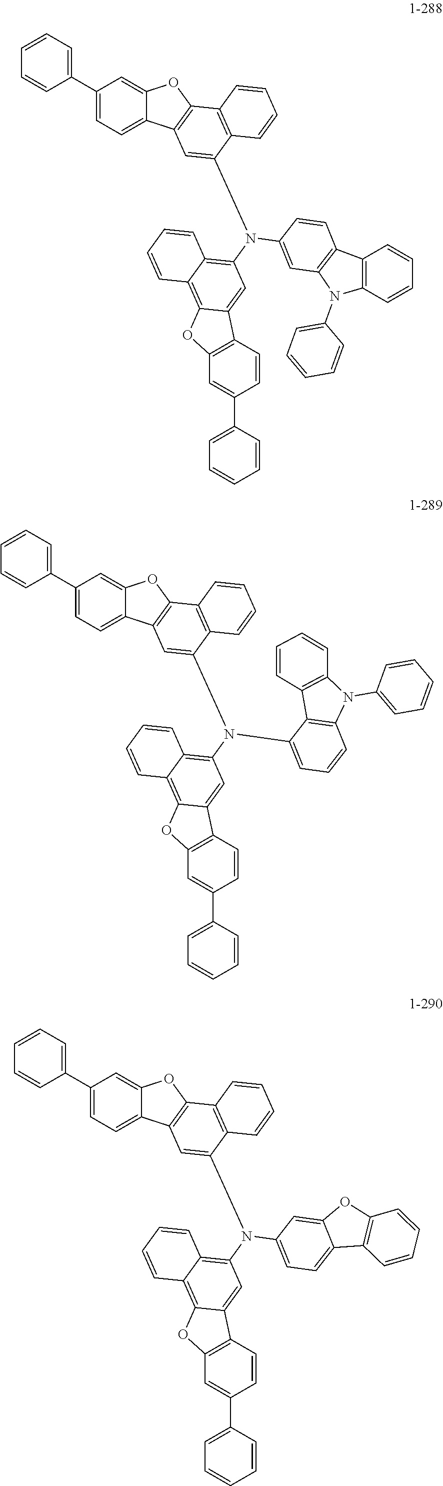 [SMILES: c1ccc(-c2ccc3c(c2)oc2c4ccccc4c(N(c4ccc5c6ccccc6n(-c6ccccc6)c5c4)c4cc5c6ccc(-c7ccccc7)cc6oc5c5ccccc45)cc32)cc1 ; c1ccc(-c2ccc3c(c2)oc2c4ccccc4c(N(c4cc5c6ccc(-c7ccccc7)cc6oc5c5ccccc45)c4cccc5c4c4ccccc4n5-c4ccccc4)cc32)cc1 ; c1ccc(-c2ccc3c(c2)oc2c4ccccc4c(N(c4ccc5c(c4)oc4ccccc45)c4cc5c6ccc(-c7ccccc7)cc6oc5c5ccccc45)cc32)cc1]

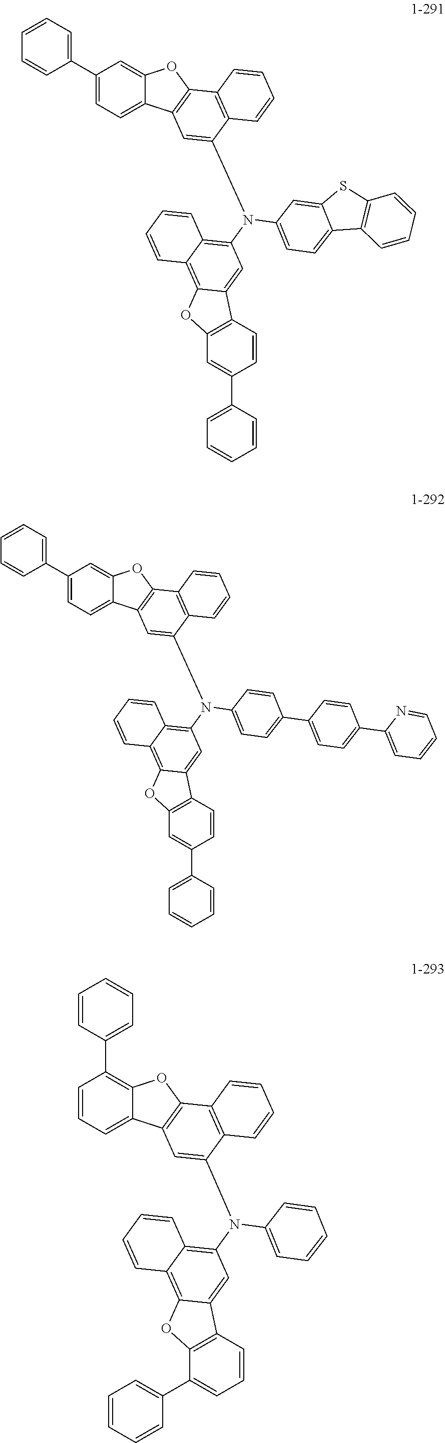 [SMILES: c1ccc(-c2ccc3c(c2)oc2c4ccccc4c(N(c4ccc5c(c4)sc4ccccc45)c4cc5c6ccc(-c7ccccc7)cc6oc5c5ccccc45)cc32)cc1 ; c1ccc(-c2ccc3c(c2)oc2c4ccccc4c(N(c4ccc(-c5ccc(-c6ccccn6)cc5)cc4)c4cc5c6ccc(-c7ccccc7)cc6oc5c5ccccc45)cc32)cc1 ; c1ccc(-c2cccc3c2oc2c4ccccc4c(N(c4ccccc4)c4cc5c6cccc(-c7ccccc7)c6oc5c5ccccc45)cc32)cc1]

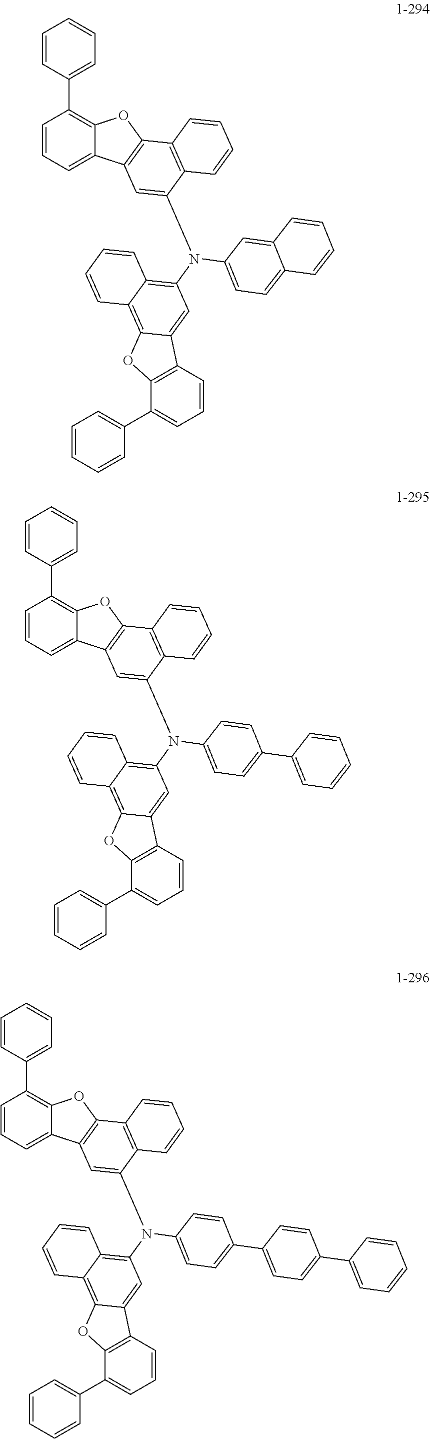 [SMILES: c1ccc(-c2cccc3c2oc2c4ccccc4c(N(c4ccc5ccccc5c4)c4cc5c6cccc(-c7ccccc7)c6oc5c5ccccc45)cc32)cc1 ; c1ccc(-c2ccc(N(c3cc4c5cccc(-c6ccccc6)c5oc4c4ccccc34)c3cc4c5cccc(-c6ccccc6)c5oc4c4ccccc34)cc2)cc1 ; c1ccc(-c2ccc(-c3ccc(N(c4cc5c6cccc(-c7ccccc7)c6oc5c5ccccc45)c4cc5c6cccc(-c7ccccc7)c6oc5c5ccccc45)cc3)cc2)cc1]

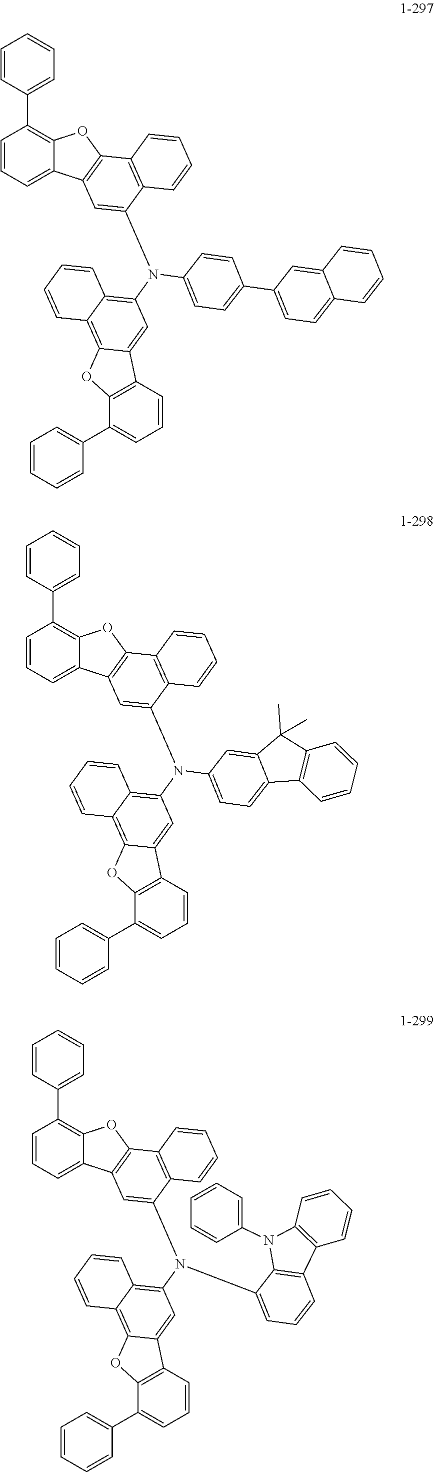 [SMILES: c1ccc(-c2cccc3c2oc2c4ccccc4c(N(c4ccc(-c5ccc6ccccc6c5)cc4)c4cc5c6cccc(-c7ccccc7)c6oc5c5ccccc45)cc32)cc1 ; CC1(C)c2ccccc2-c2ccc(N(c3cc4c5cccc(-c6ccccc6)c5oc4c4ccccc34)c3cc4c5cccc(-c6ccccc6)c5oc4c4ccccc34)cc21 ; c1ccc(-c2cccc3c2oc2c4ccccc4c(N(c4cc5c6cccc(-c7ccccc7)c6oc5c5ccccc45)c4cccc5c6ccccc6n(-c6ccccc6)c45)cc32)cc1]

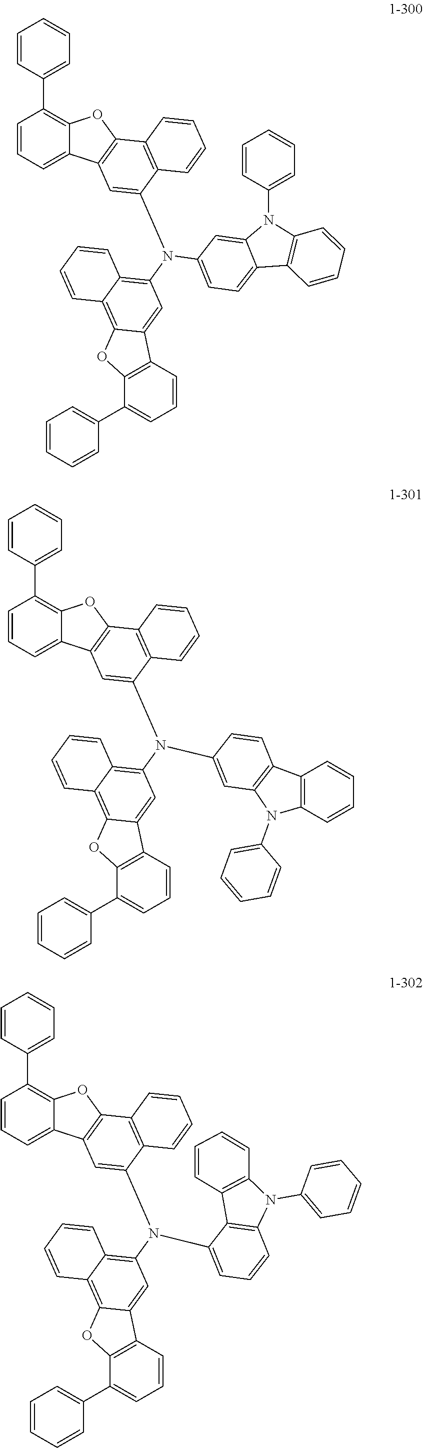 [SMILES: c1ccc(-c2cccc3c2oc2c4ccccc4c(N(c4ccc5c6ccccc6n(-c6ccccc6)c5c4)c4cc5c6cccc(-c7ccccc7)c6oc5c5ccccc45)cc32)cc1 ; c1ccc(-c2cccc3c2oc2c4ccccc4c(N(c4ccc5c6ccccc6n(-c6ccccc6)c5c4)c4cc5c6cccc(-c7ccccc7)c6oc5c5ccccc45)cc32)cc1 ; c1ccc(-c2cccc3c2oc2c4ccccc4c(N(c4cc5c6cccc(-c7ccccc7)c6oc5c5ccccc45)c4cccc5c4c4ccccc4n5-c4ccccc4)cc32)cc1]

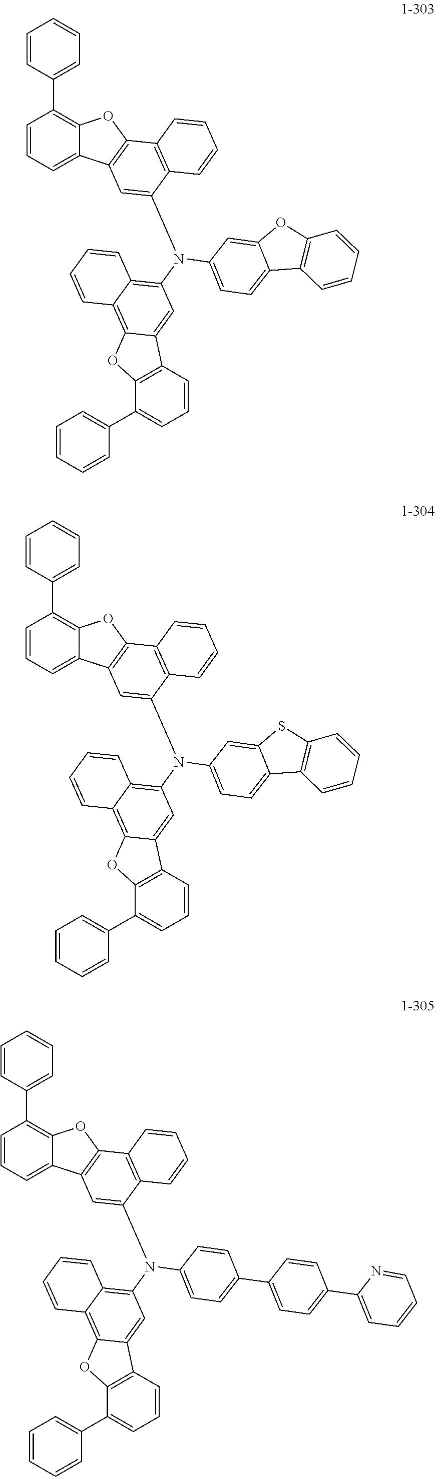 [SMILES: c1ccc(-c2cccc3c2oc2c4ccccc4c(N(c4ccc5c(c4)oc4ccccc45)c4cc5c6cccc(-c7ccccc7)c6oc5c5ccccc45)cc32)cc1 ; c1ccc(-c2cccc3c2oc2c4ccccc4c(N(c4ccc5c(c4)sc4ccccc45)c4cc5c6cccc(-c7ccccc7)c6oc5c5ccccc45)cc32)cc1 ; c1ccc(-c2cccc3c2oc2c4ccccc4c(N(c4ccc(-c5ccc(-c6ccccn6)cc5)cc4)c4cc5c6cccc(-c7ccccc7)c6oc5c5ccccc45)cc32)cc1]

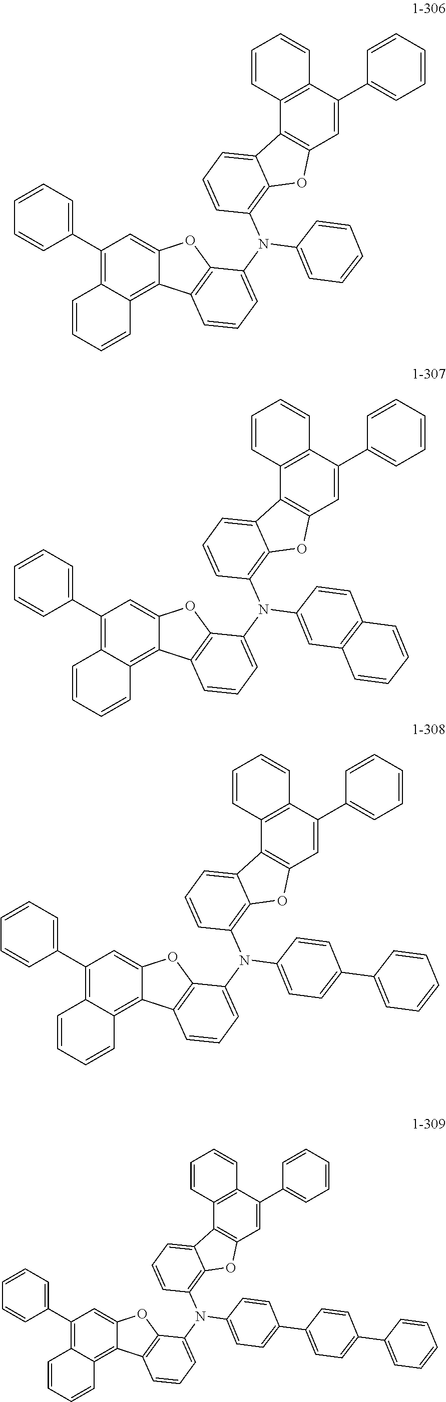 [SMILES: c1ccc(-c2cc3oc4c(N(c5ccccc5)c5cccc6c5oc5cc(-c7ccccc7)c7ccccc7c56)cccc4c3c3ccccc23)cc1 ; c1ccc(-c2cc3oc4c(N(c5ccc6ccccc6c5)c5cccc6c5oc5cc(-c7ccccc7)c7ccccc7c56)cccc4c3c3ccccc23)cc1 ; c1ccc(-c2ccc(N(c3cccc4c3oc3cc(-c5ccccc5)c5ccccc5c34)c3cccc4c3oc3cc(-c5ccccc5)c5ccccc5c34)cc2)cc1 ; c1ccc(-c2ccc(-c3ccc(N(c4cccc5c4oc4cc(-c6ccccc6)c6ccccc6c45)c4cccc5c4oc4cc(-c6ccccc6)c6ccccc6c45)cc3)cc2)cc1]

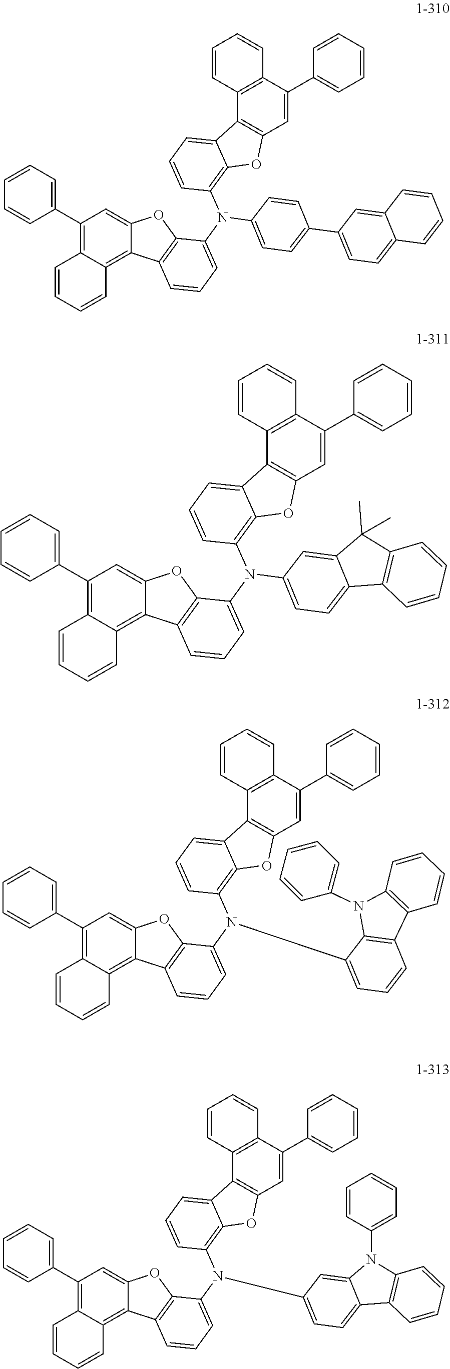 [SMILES: c1ccc(-c2cc3oc4c(N(c5ccc(-c6ccc7ccccc7c6)cc5)c5cccc6c5oc5cc(-c7ccccc7)c7ccccc7c56)cccc4c3c3ccccc23)cc1 ; CC1(C)c2ccccc2-c2ccc(N(c3cccc4c3oc3cc(-c5ccccc5)c5ccccc5c34)c3cccc4c3oc3cc(-c5ccccc5)c5ccccc5c34)cc21 ; c1ccc(-c2cc3oc4c(N(c5cccc6c5oc5cc(-c7ccccc7)c7ccccc7c56)c5cccc6c7ccccc7n(-c7ccccc7)c56)cccc4c3c3ccccc23)cc1 ; c1ccc(-c2cc3oc4c(N(c5ccc6c7ccccc7n(-c7ccccc7)c6c5)c5cccc6c5oc5cc(-c7ccccc7)c7ccccc7c56)cccc4c3c3ccccc23)cc1]

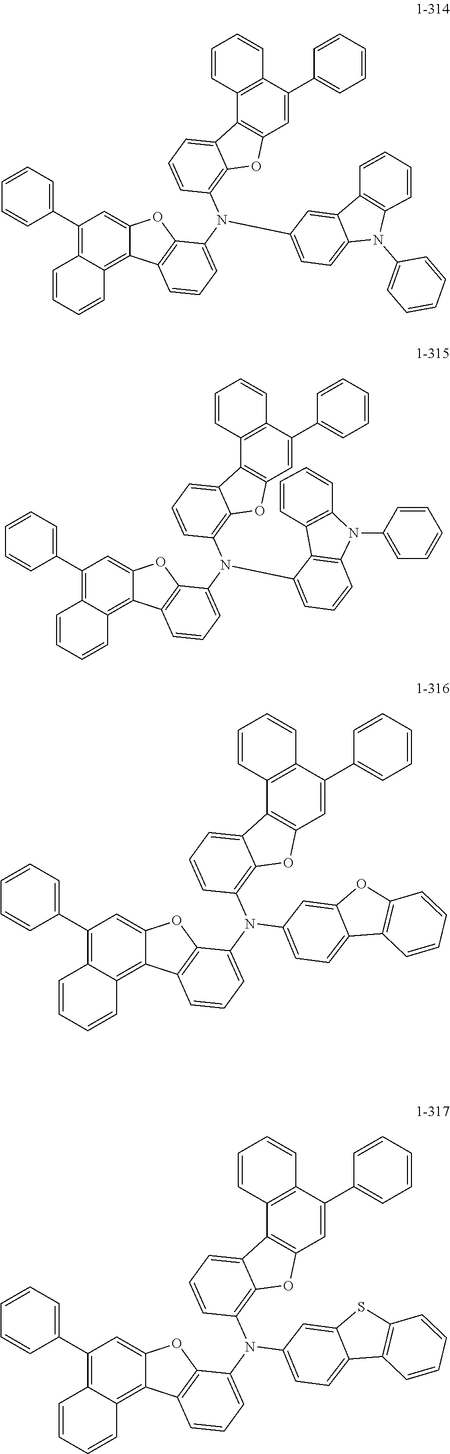 [SMILES: c1ccc(-c2cc3oc4c(N(c5ccc6c(c5)c5ccccc5n6-c5ccccc5)c5cccc6c5oc5cc(-c7ccccc7)c7ccccc7c56)cccc4c3c3ccccc23)cc1 ; c1ccc(-c2cc3oc4c(N(c5cccc6c5oc5cc(-c7ccccc7)c7ccccc7c56)c5cccc6c5c5ccccc5n6-c5ccccc5)cccc4c3c3ccccc23)cc1 ; c1ccc(-c2cc3oc4c(N(c5ccc6c(c5)oc5ccccc56)c5cccc6c5oc5cc(-c7ccccc7)c7ccccc7c56)cccc4c3c3ccccc23)cc1 ; c1ccc(-c2cc3oc4c(N(c5ccc6c(c5)sc5ccccc56)c5cccc6c5oc5cc(-c7ccccc7)c7ccccc7c56)cccc4c3c3ccccc23)cc1]

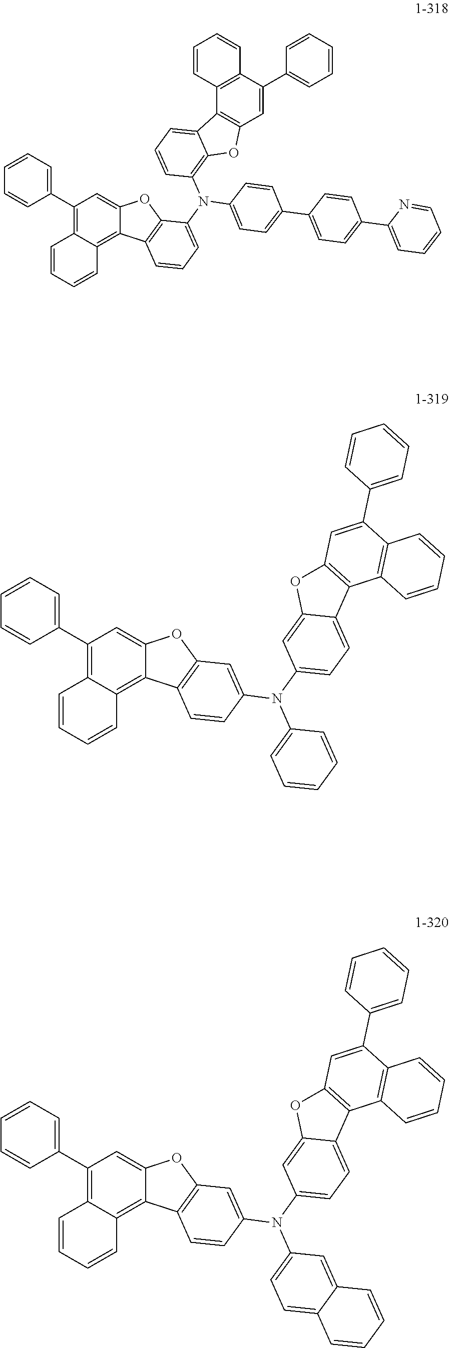 [SMILES: c1ccc(-c2cc3oc4c(N(c5ccc(-c6ccc(-c7ccccn7)cc6)cc5)c5cccc6c5oc5cc(-c7ccccc7)c7ccccc7c56)cccc4c3c3ccccc23)cc1 ; c1ccc(-c2cc3oc4cc(N(c5ccccc5)c5ccc6c(c5)oc5cc(-c7ccccc7)c7ccccc7c56)ccc4c3c3ccccc23)cc1 ; c1ccc(-c2cc3oc4cc(N(c5ccc6ccccc6c5)c5ccc6c(c5)oc5cc(-c7ccccc7)c7ccccc7c56)ccc4c3c3ccccc23)cc1]

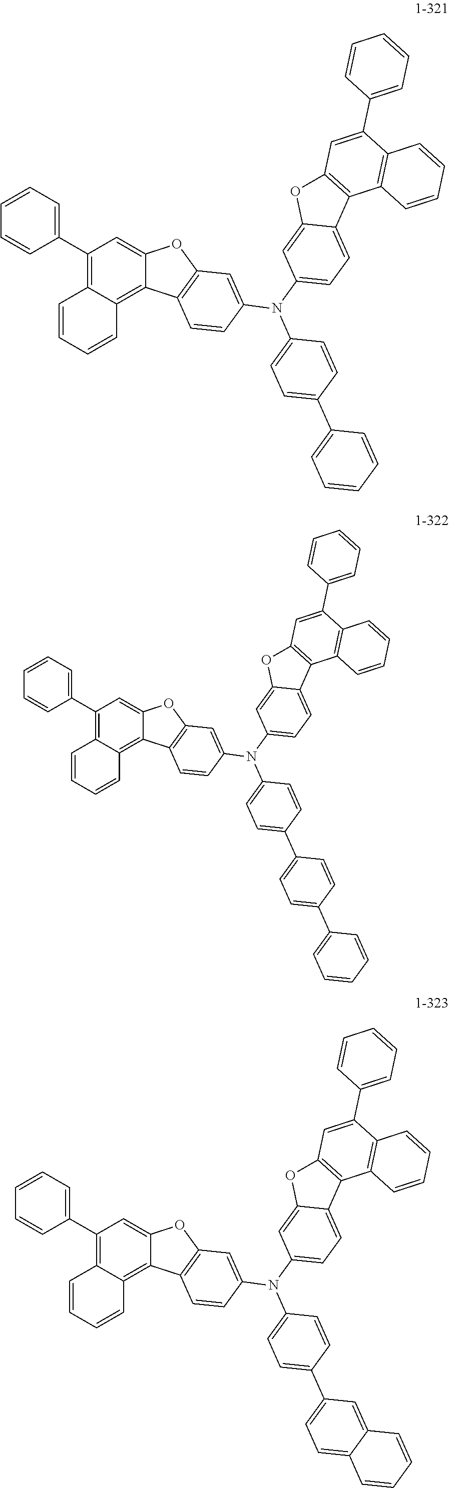

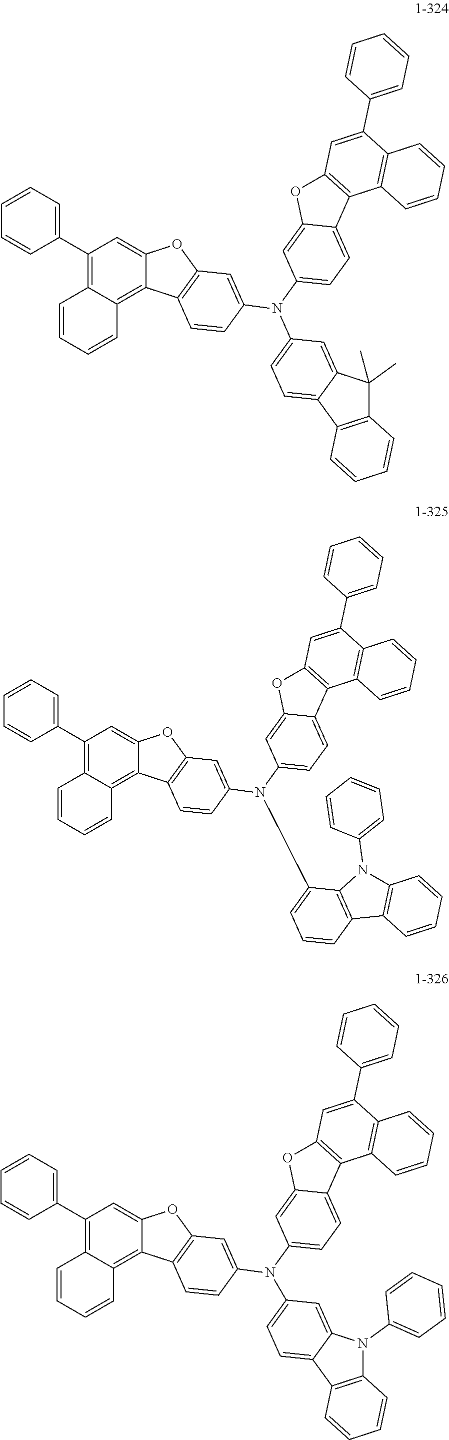 [SMILES: CC1(C)c2ccccc2-c2ccc(N(c3ccc4c(c3)oc3cc(-c5ccccc5)c5ccccc5c34)c3ccc4c(c3)oc3cc(-c5ccccc5)c5ccccc5c34)cc21 ; c1ccc(-c2cc3oc4cc(N(c5ccc6c(c5)oc5cc(-c7ccccc7)c7ccccc7c56)c5cccc6c7ccccc7n(-c7ccccc7)c56)ccc4c3c3ccccc23)cc1 ; c1ccc(-c2cc3oc4cc(N(c5ccc6c(c5)oc5cc(-c7ccccc7)c7ccccc7c56)c5ccc6c7ccccc7n(-c7ccccc7)c6c5)ccc4c3c3ccccc23)cc1]

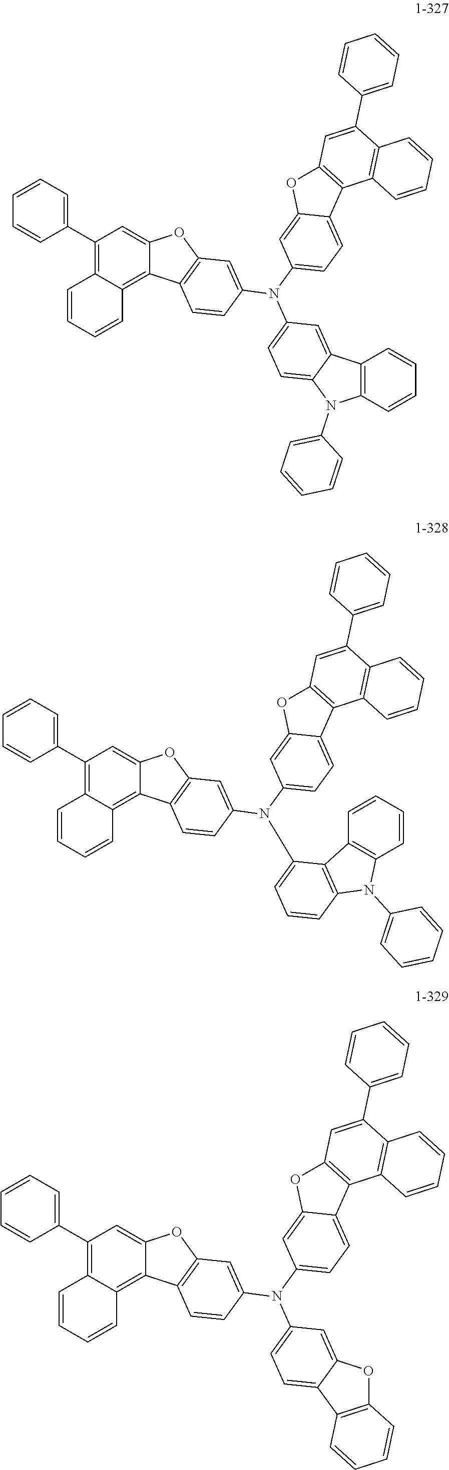 [SMILES: c1ccc(-c2cc3oc4cc(N(c5ccc6c(c5)oc5cc(-c7ccccc7)c7ccccc7c56)c5ccc6c(c5)c5ccccc5n6-c5ccccc5)ccc4c3c3ccccc23)cc1 ; c1ccc(-c2cc3oc4cc(N(c5ccc6c(c5)oc5cc(-c7ccccc7)c7ccccc7c56)c5cccc6c5c5ccccc5n6-c5ccccc5)ccc4c3c3ccccc23)cc1 ; c1ccc(-c2cc3oc4cc(N(c5ccc6c(c5)oc5ccccc56)c5ccc6c(c5)oc5cc(-c7ccccc7)c7ccccc7c56)ccc4c3c3ccccc23)cc1]

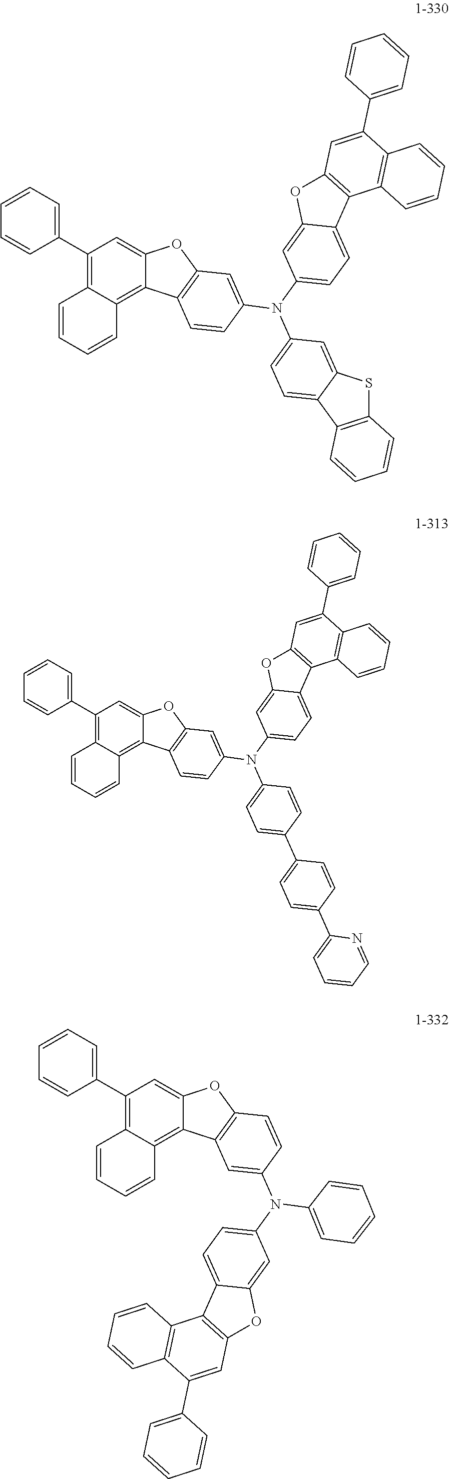 [SMILES: c1ccc(-c2cc3oc4cc(N(c5ccc6c(c5)oc5cc(-c7ccccc7)c7ccccc7c56)c5ccc6c(c5)sc5ccccc56)ccc4c3c3ccccc23)cc1 ; c1ccc(-c2cc3oc4cc(N(c5ccc(-c6ccc(-c7ccccn7)cc6)cc5)c5ccc6c(c5)oc5cc(-c7ccccc7)c7ccccc7c56)ccc4c3c3ccccc23)cc1 ; c1ccc(-c2cc3oc4cc(N(c5ccccc5)c5ccc6oc7cc(-c8ccccc8)c8ccccc8c7c6c5)ccc4c3c3ccccc23)cc1]

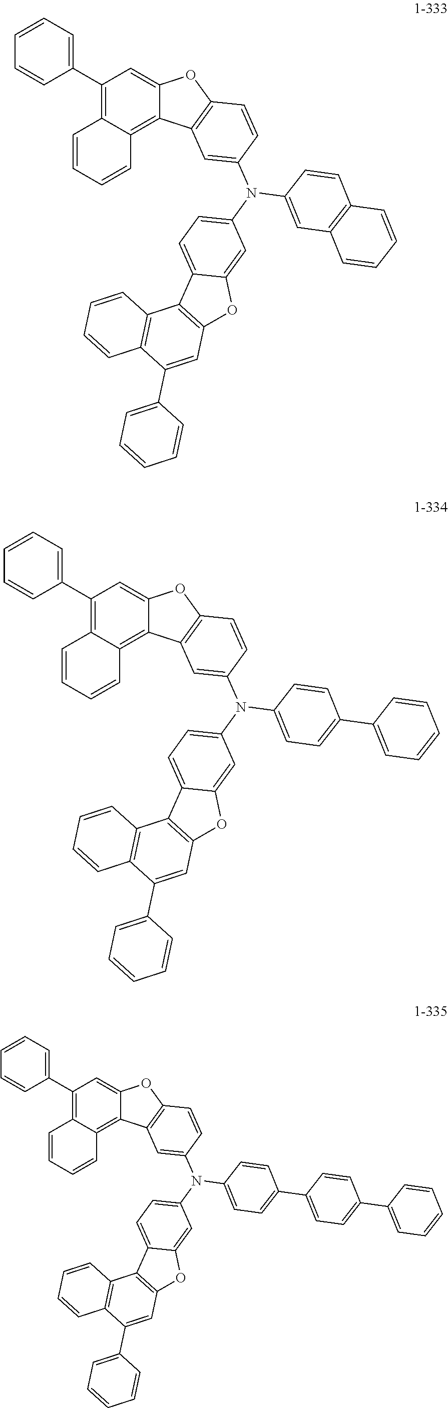 [SMILES: c1ccc(-c2cc3oc4cc(N(c5ccc6ccccc6c5)c5ccc6oc7cc(-c8ccccc8)c8ccccc8c7c6c5)ccc4c3c3ccccc23)cc1 ; c1ccc(-c2ccc(N(c3ccc4c(c3)oc3cc(-c5ccccc5)c5ccccc5c34)c3ccc4oc5cc(-c6ccccc6)c6ccccc6c5c4c3)cc2)cc1 ; c1ccc(-c2ccc(-c3ccc(N(c4ccc5c(c4)oc4cc(-c6ccccc6)c6ccccc6c45)c4ccc5oc6cc(-c7ccccc7)c7ccccc7c6c5c4)cc3)cc2)cc1]

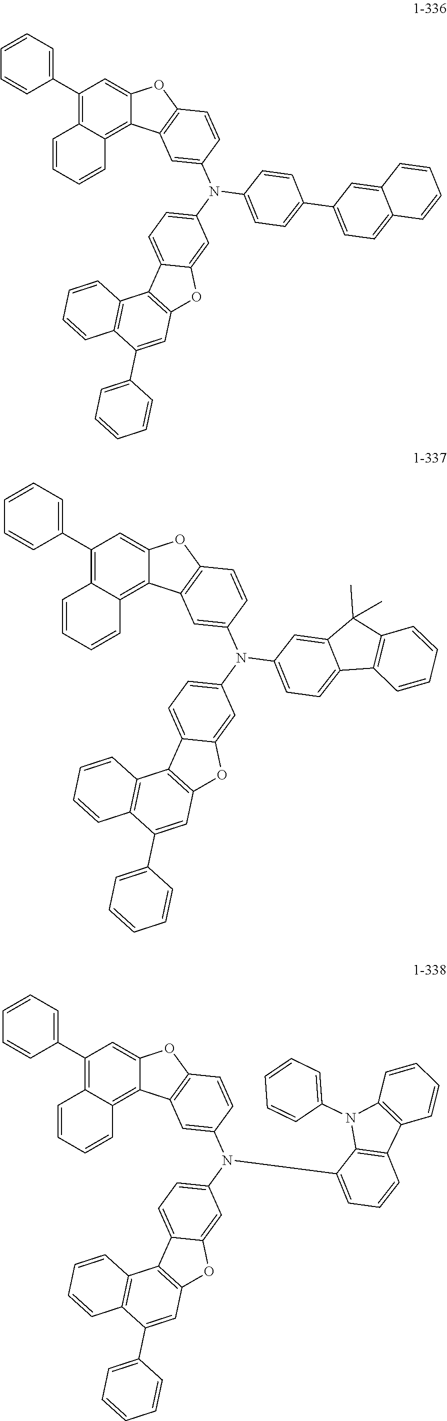 [SMILES: c1ccc(-c2cc3oc4cc(N(c5ccc(-c6ccc7ccccc7c6)cc5)c5ccc6oc7cc(-c8ccccc8)c8ccccc8c7c6c5)ccc4c3c3ccccc23)cc1 ; CC1(C)c2ccccc2-c2ccc(N(c3ccc4c(c3)oc3cc(-c5ccccc5)c5ccccc5c34)c3ccc4oc5cc(-c6ccccc6)c6ccccc6c5c4c3)cc21 ; c1ccc(-c2cc3oc4cc(N(c5ccc6oc7cc(-c8ccccc8)c8ccccc8c7c6c5)c5cccc6c7ccccc7n(-c7ccccc7)c56)ccc4c3c3ccccc23)cc1]

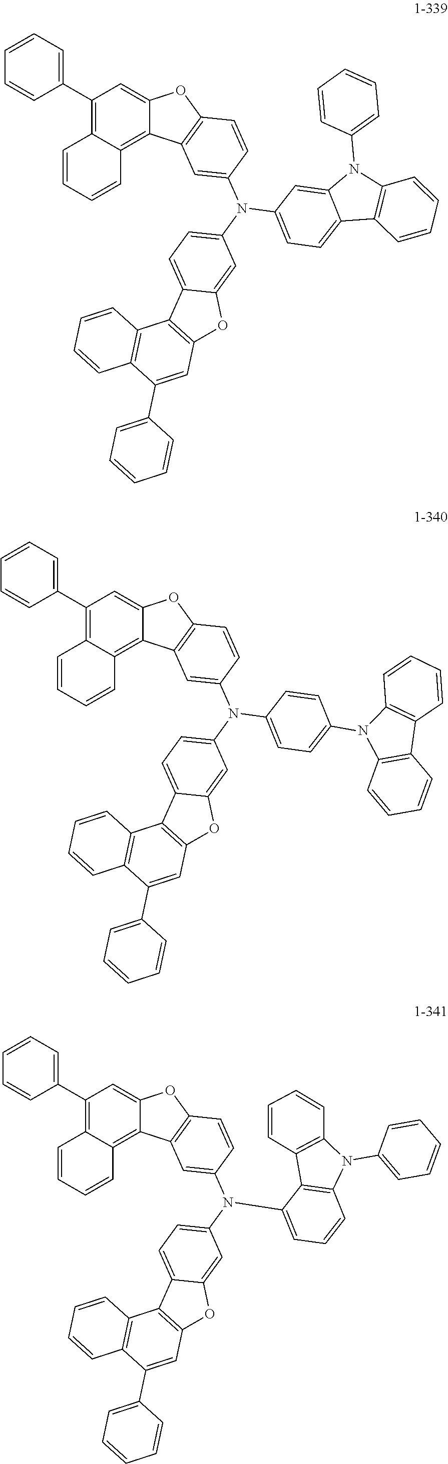 [SMILES: c1ccc(-c2cc3oc4cc(N(c5ccc6oc7cc(-c8ccccc8)c8ccccc8c7c6c5)c5ccc6c7ccccc7n(-c7ccccc7)c6c5)ccc4c3c3ccccc23)cc1 ; c1ccc(-c2cc3oc4cc(N(c5ccc(-n6c7ccccc7c7ccccc76)cc5)c5ccc6oc7cc(-c8ccccc8)c8ccccc8c7c6c5)ccc4c3c3ccccc23)cc1 ; c1ccc(-c2cc3oc4cc(N(c5ccc6oc7cc(-c8ccccc8)c8ccccc8c7c6c5)c5cccc6c5c5ccccc5n6-c5ccccc5)ccc4c3c3ccccc23)cc1]

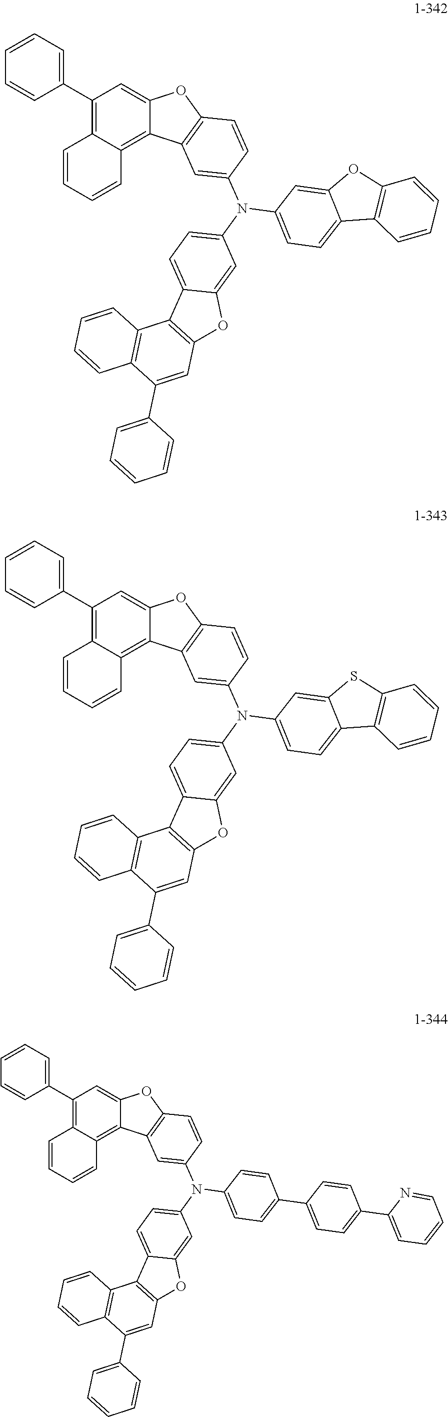 [SMILES: c1ccc(-c2cc3oc4cc(N(c5ccc6c(c5)oc5ccccc56)c5ccc6oc7cc(-c8ccccc8)c8ccccc8c7c6c5)ccc4c3c3ccccc23)cc1 ; c1ccc(-c2cc3oc4cc(N(c5ccc6c(c5)sc5ccccc56)c5ccc6oc7cc(-c8ccccc8)c8ccccc8c7c6c5)ccc4c3c3ccccc23)cc1 ; c1ccc(-c2cc3oc4cc(N(c5ccc(-c6ccc(-c7ccccn7)cc6)cc5)c5ccc6oc7cc(-c8ccccc8)c8ccccc8c7c6c5)ccc4c3c3ccccc23)cc1]

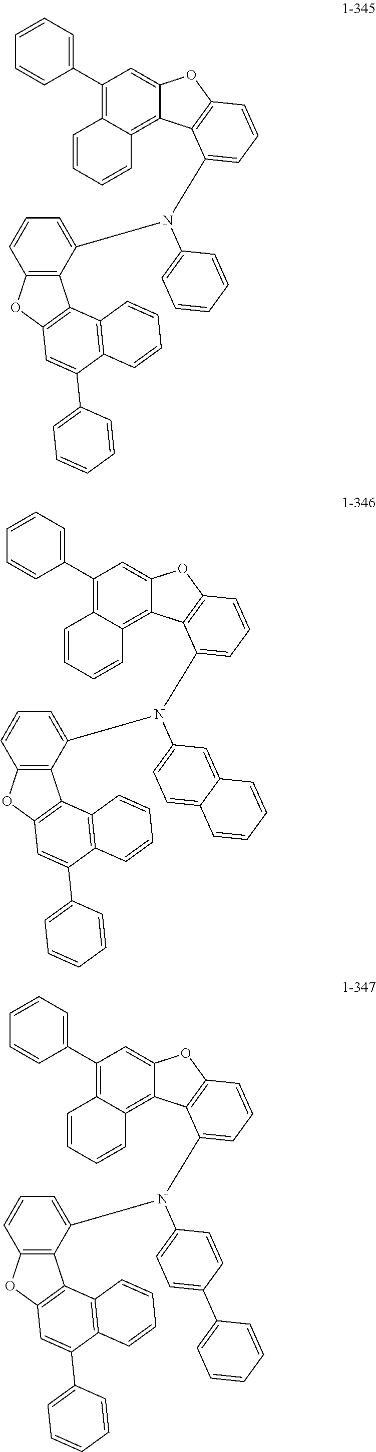

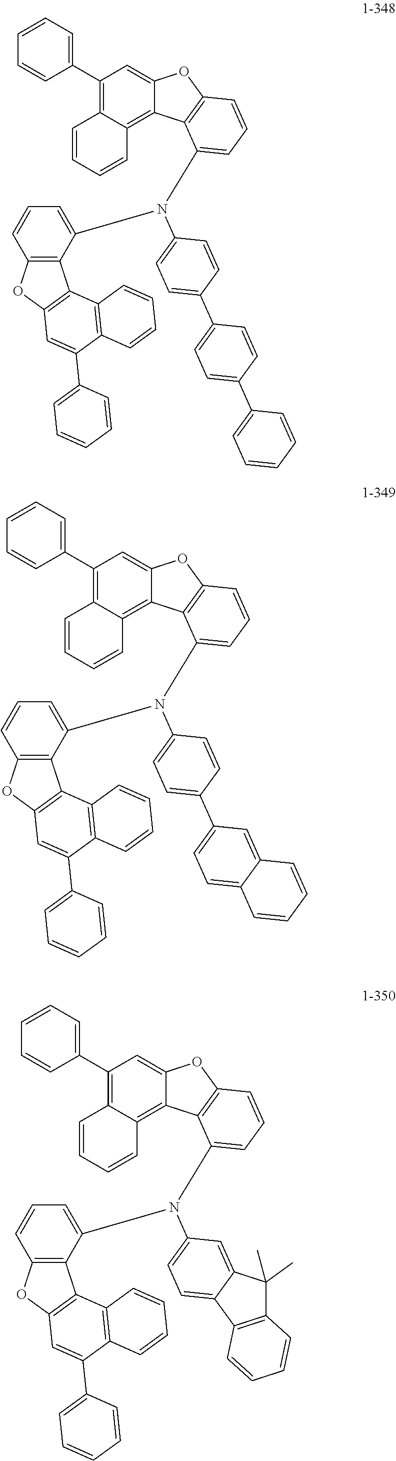 [SMILES: c1ccc(-c2ccc(-c3ccc(N(c4cccc5oc6cc(-c7ccccc7)c7ccccc7c6c45)c4cccc5oc6cc(-c7ccccc7)c7ccccc7c6c45)cc3)cc2)cc1 ; c1ccc(-c2cc3oc4cccc(N(c5ccc(-c6ccc7ccccc7c6)cc5)c5cccc6oc7cc(-c8ccccc8)c8ccccc8c7c56)c4c3c3ccccc23)cc1 ; CC1(C)c2ccccc2-c2ccc(N(c3cccc4oc5cc(-c6ccccc6)c6ccccc6c5c34)c3cccc4oc5cc(-c6ccccc6)c6ccccc6c5c34)cc21]

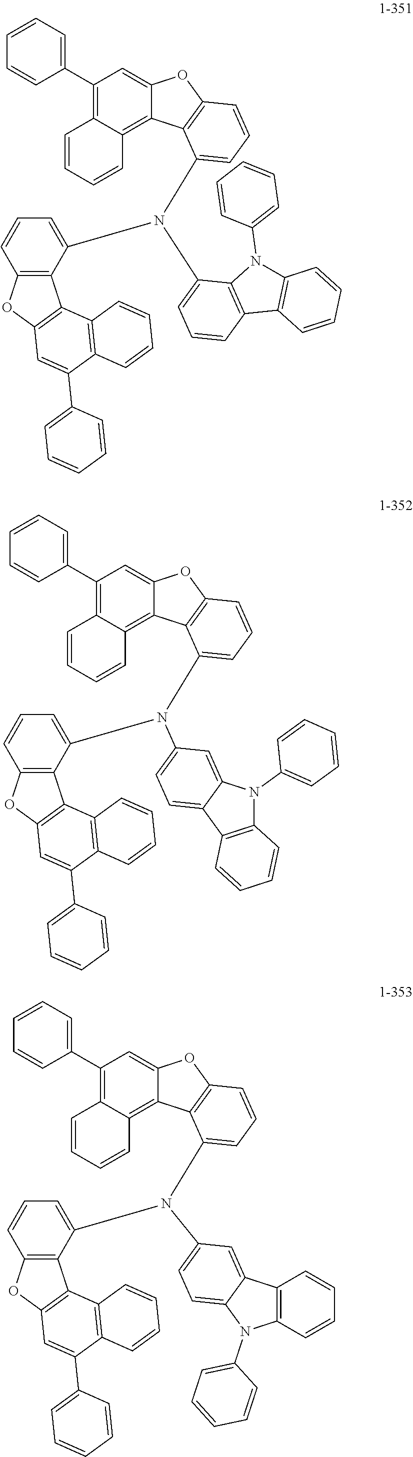

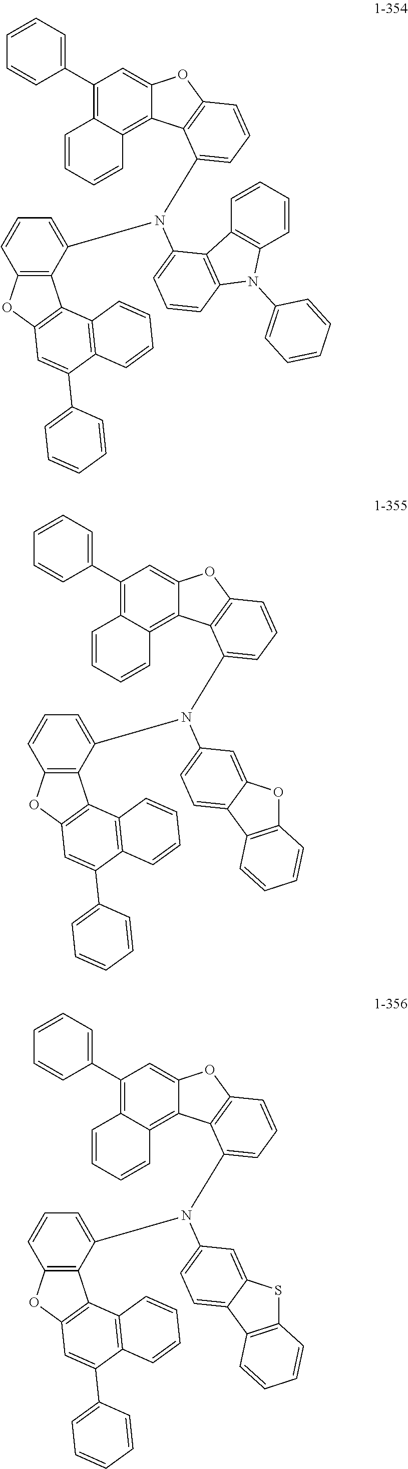

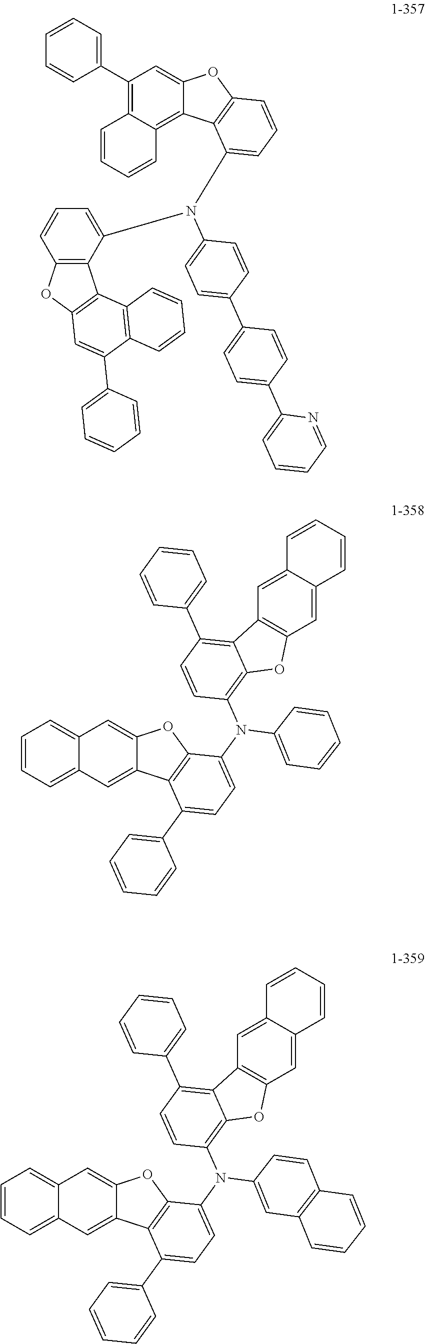 [SMILES: c1ccc(-c2cc3oc4cccc(N(c5ccc(-c6ccc(-c7ccccn7)cc6)cc5)c5cccc6oc7cc(-c8ccccc8)c8ccccc8c7c56)c4c3c3ccccc23)cc1 ; c1ccc(-c2ccc(N(c3ccccc3)c3ccc(-c4ccccc4)c4c3oc3cc5ccccc5cc34)c3oc4cc5ccccc5cc4c23)cc1 ; c1ccc(-c2ccc(N(c3ccc4ccccc4c3)c3ccc(-c4ccccc4)c4c3oc3cc5ccccc5cc34)c3oc4cc5ccccc5cc4c23)cc1]

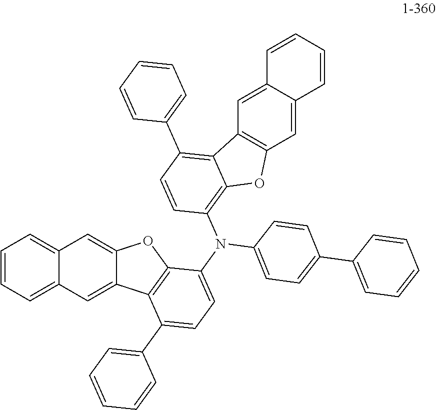 [SMILES: c1ccc(-c2ccc(N(c3ccc(-c4ccccc4)c4c3oc3cc5ccccc5cc34)c3ccc(-c4ccccc4)c4c3oc3cc5ccccc5cc34)cc2)cc1]

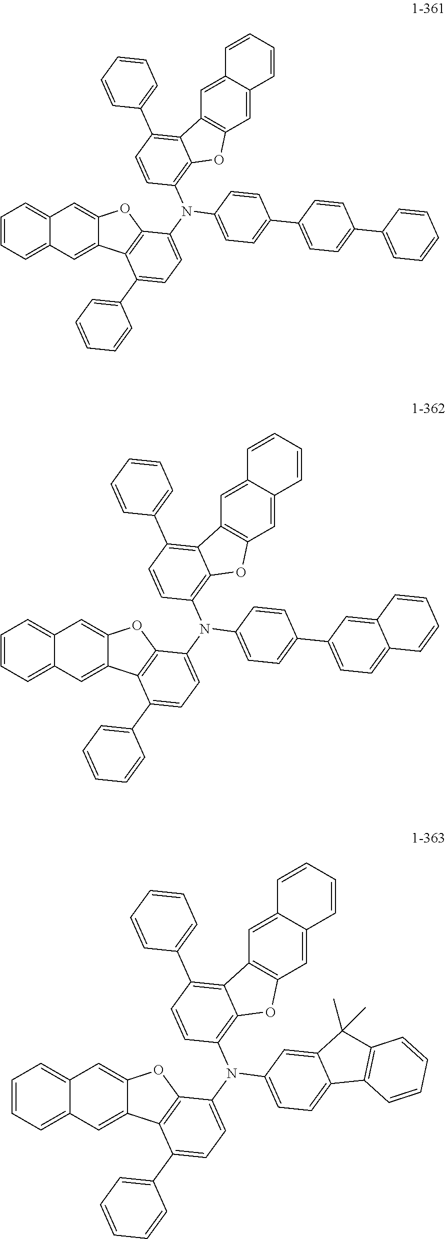 [SMILES: c1ccc(-c2ccc(-c3ccc(N(c4ccc(-c5ccccc5)c5c4oc4cc6ccccc6cc45)c4ccc(-c5ccccc5)c5c4oc4cc6ccccc6cc45)cc3)cc2)cc1 ; c1ccc(-c2ccc(N(c3ccc(-c4ccc5ccccc5c4)cc3)c3ccc(-c4ccccc4)c4c3oc3cc5ccccc5cc34)c3oc4cc5ccccc5cc4c23)cc1 ; CC1(C)c2ccccc2-c2ccc(N(c3ccc(-c4ccccc4)c4c3oc3cc5ccccc5cc34)c3ccc(-c4ccccc4)c4c3oc3cc5ccccc5cc34)cc21]

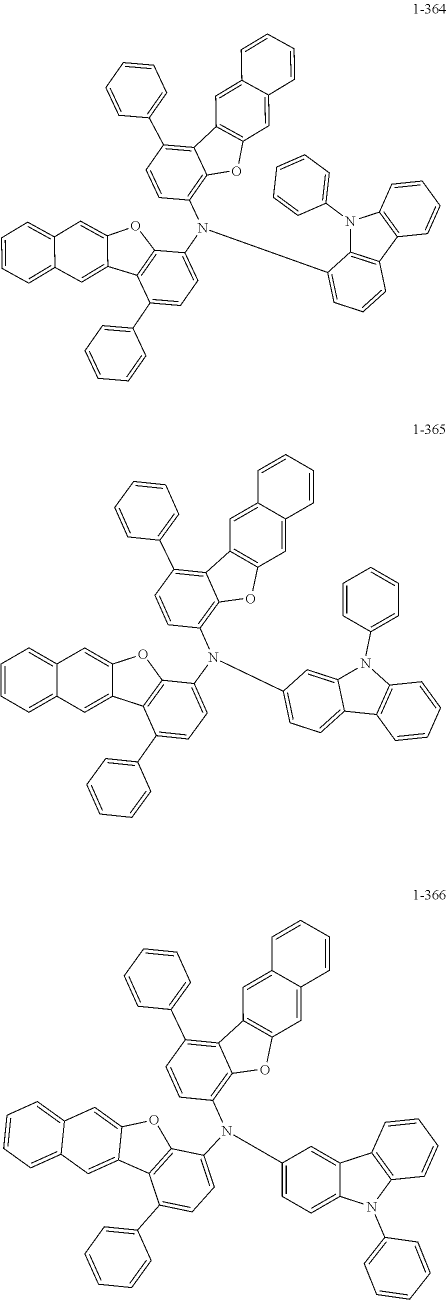 [SMILES: c1ccc(-c2ccc(N(c3ccc(-c4ccccc4)c4c3oc3cc5ccccc5cc34)c3cccc4c5ccccc5n(-c5ccccc5)c34)c3oc4cc5ccccc5cc4c23)cc1 ; c1ccc(-c2ccc(N(c3ccc4c5ccccc5n(-c5ccccc5)c4c3)c3ccc(-c4ccccc4)c4c3oc3cc5ccccc5cc34)c3oc4cc5ccccc5cc4c23)cc1 ; c1ccc(-c2ccc(N(c3ccc4c(c3)c3ccccc3n4-c3ccccc3)c3ccc(-c4ccccc4)c4c3oc3cc5ccccc5cc34)c3oc4cc5ccccc5cc4c23)cc1]

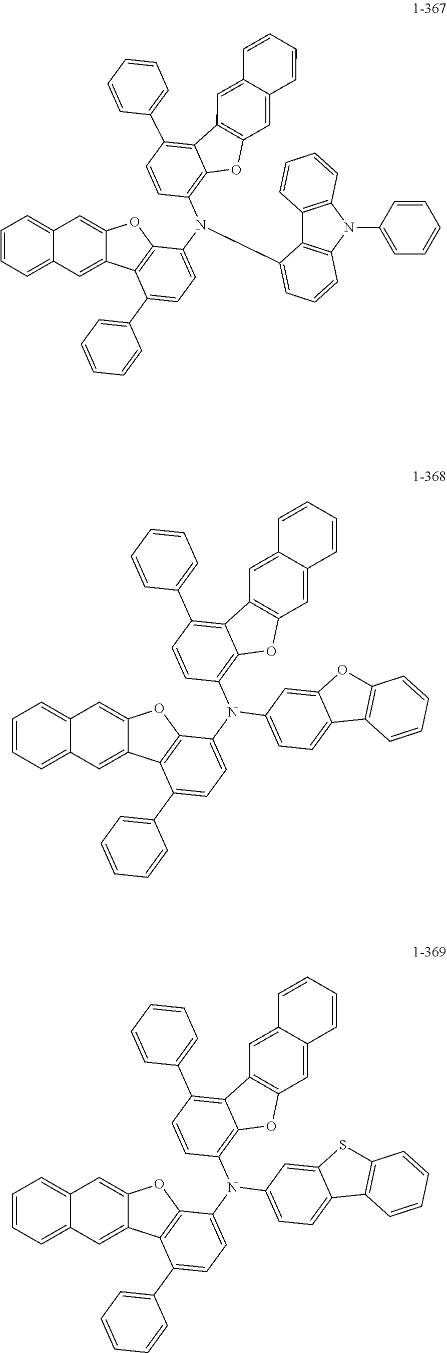 [SMILES: c1ccc(-c2ccc(N(c3ccc(-c4ccccc4)c4c3oc3cc5ccccc5cc34)c3cccc4c3c3ccccc3n4-c3ccccc3)c3oc4cc5ccccc5cc4c23)cc1 ; c1ccc(-c2ccc(N(c3ccc4c(c3)oc3ccccc34)c3ccc(-c4ccccc4)c4c3oc3cc5ccccc5cc34)c3oc4cc5ccccc5cc4c23)cc1 ; c1ccc(-c2ccc(N(c3ccc4c(c3)sc3ccccc34)c3ccc(-c4ccccc4)c4c3oc3cc5ccccc5cc34)c3oc4cc5ccccc5cc4c23)cc1]

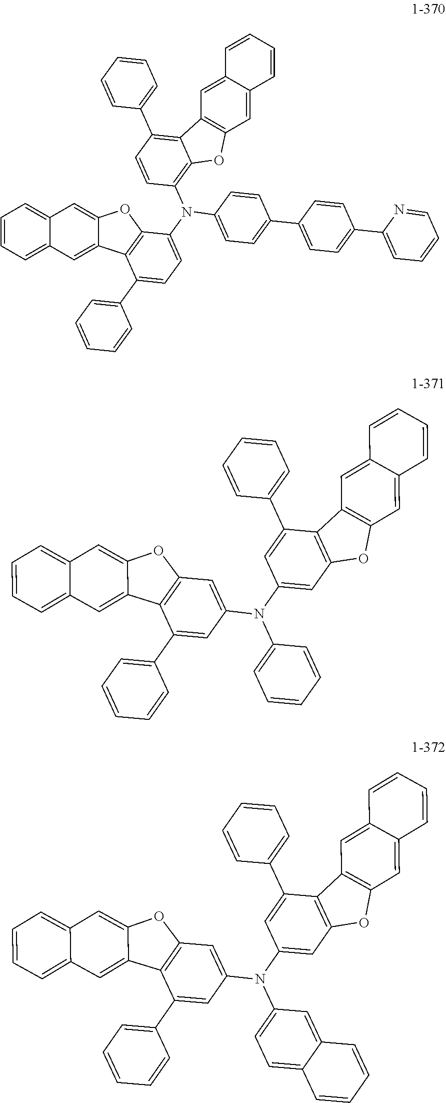 [SMILES: c1ccc(-c2ccc(N(c3ccc(-c4ccc(-c5ccccn5)cc4)cc3)c3ccc(-c4ccccc4)c4c3oc3cc5ccccc5cc34)c3oc4cc5ccccc5cc4c23)cc1 ; c1ccc(-c2cc(N(c3ccccc3)c3cc(-c4ccccc4)c4c(c3)oc3cc5ccccc5cc34)cc3oc4cc5ccccc5cc4c23)cc1 ; c1ccc(-c2cc(N(c3ccc4ccccc4c3)c3cc(-c4ccccc4)c4c(c3)oc3cc5ccccc5cc34)cc3oc4cc5ccccc5cc4c23)cc1]

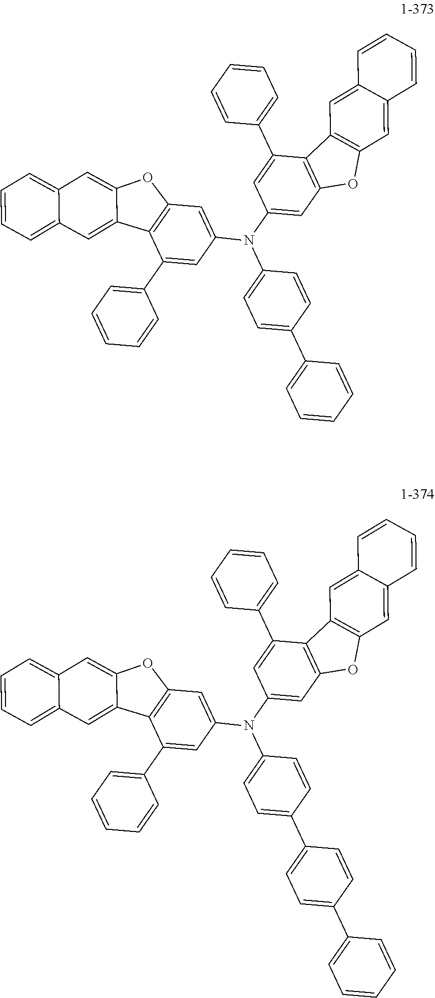 [SMILES: c1ccc(-c2ccc(N(c3cc(-c4ccccc4)c4c(c3)oc3cc5ccccc5cc34)c3cc(-c4ccccc4)c4c(c3)oc3cc5ccccc5cc34)cc2)cc1 ; c1ccc(-c2ccc(-c3ccc(N(c4cc(-c5ccccc5)c5c(c4)oc4cc6ccccc6cc45)c4cc(-c5ccccc5)c5c(c4)oc4cc6ccccc6cc45)cc3)cc2)cc1]

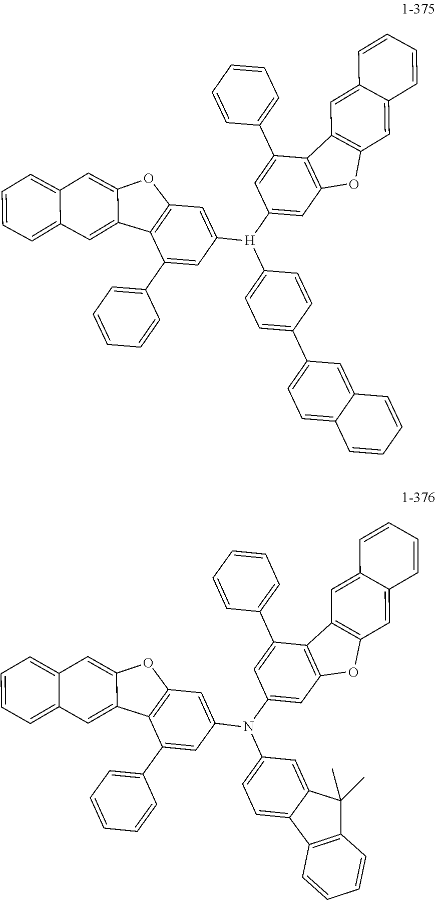

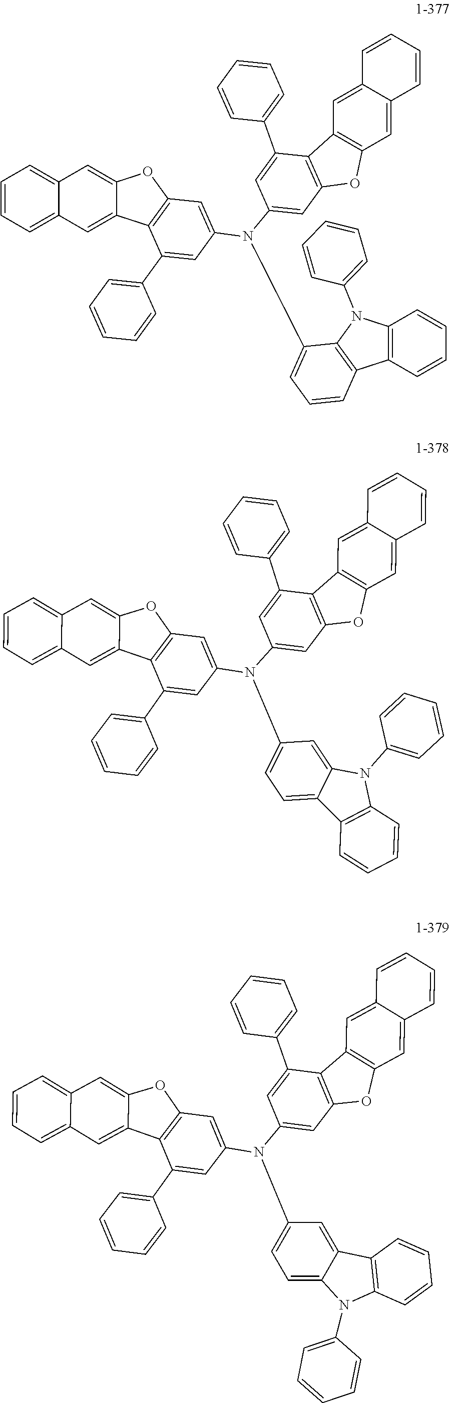 [SMILES: c1ccc(-c2cc(N(c3cc(-c4ccccc4)c4c(c3)oc3cc5ccccc5cc34)c3cccc4c5ccccc5n(-c5ccccc5)c34)cc3oc4cc5ccccc5cc4c23)cc1 ; c1ccc(-c2cc(N(c3cc(-c4ccccc4)c4c(c3)oc3cc5ccccc5cc34)c3ccc4c5ccccc5n(-c5ccccc5)c4c3)cc3oc4cc5ccccc5cc4c23)cc1 ; c1ccc(-c2cc(N(c3cc(-c4ccccc4)c4c(c3)oc3cc5ccccc5cc34)c3ccc4c(c3)c3ccccc3n4-c3ccccc3)cc3oc4cc5ccccc5cc4c23)cc1]

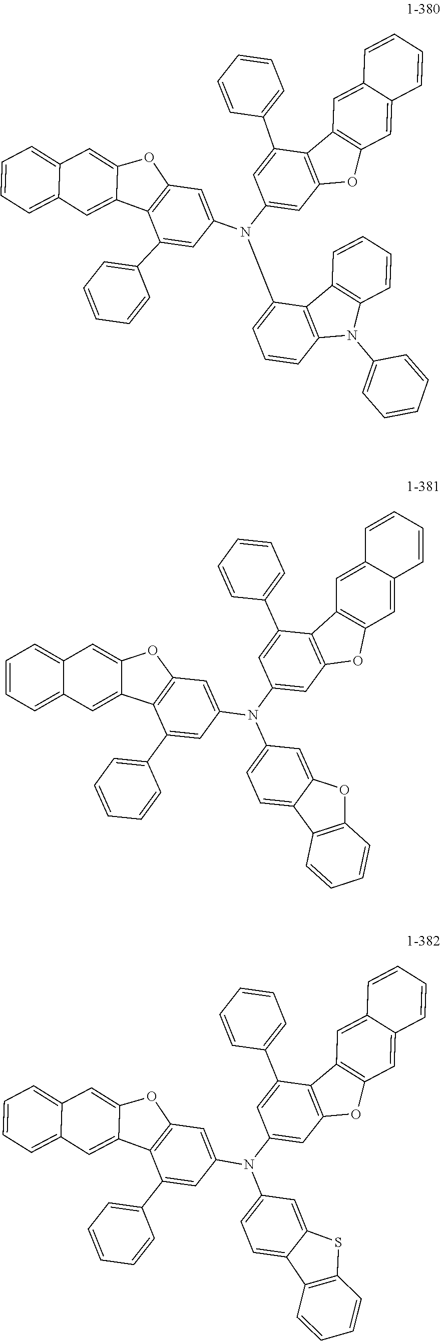 [SMILES: c1ccc(-c2cc(N(c3cc(-c4ccccc4)c4c(c3)oc3cc5ccccc5cc34)c3cccc4c3c3ccccc3n4-c3ccccc3)cc3oc4cc5ccccc5cc4c23)cc1 ; c1ccc(-c2cc(N(c3ccc4c(c3)oc3ccccc34)c3cc(-c4ccccc4)c4c(c3)oc3cc5ccccc5cc34)cc3oc4cc5ccccc5cc4c23)cc1 ; c1ccc(-c2cc(N(c3cc(-c4ccccc4)c4c(c3)oc3cc5ccccc5cc34)c3ccc4c(c3)sc3ccccc34)cc3oc4cc5ccccc5cc4c23)cc1]

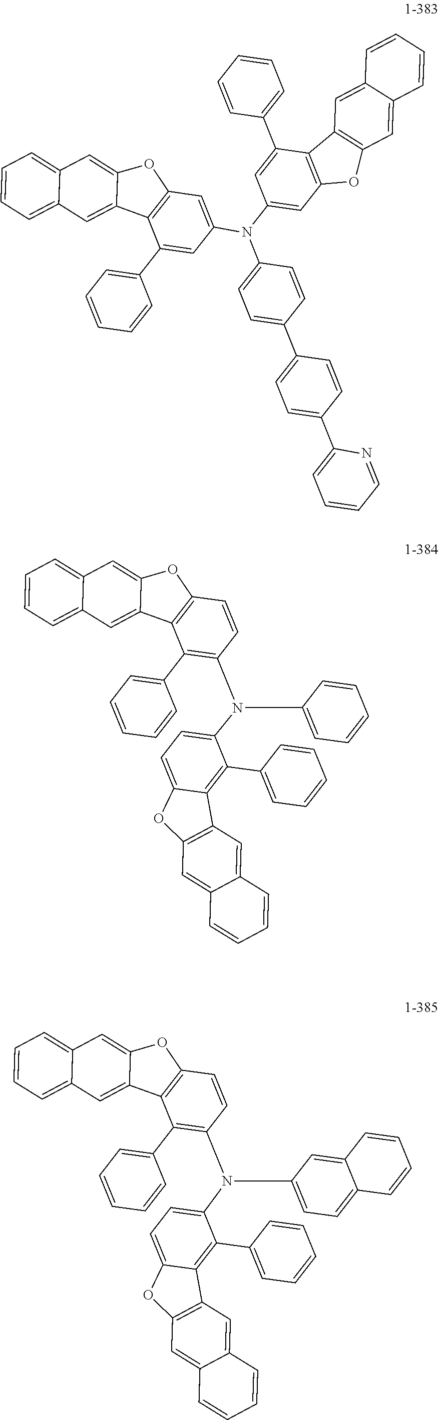 [SMILES: c1ccc(-c2cc(N(c3ccc(-c4ccc(-c5ccccn5)cc4)cc3)c3cc(-c4ccccc4)c4c(c3)oc3cc5ccccc5cc34)cc3oc4cc5ccccc5cc4c23)cc1 ; c1ccc(-c2c(N(c3ccccc3)c3ccc4oc5cc6ccccc6cc5c4c3-c3ccccc3)ccc3oc4cc5ccccc5cc4c23)cc1 ; c1ccc(-c2c(N(c3ccc4ccccc4c3)c3ccc4oc5cc6ccccc6cc5c4c3-c3ccccc3)ccc3oc4cc5ccccc5cc4c23)cc1]

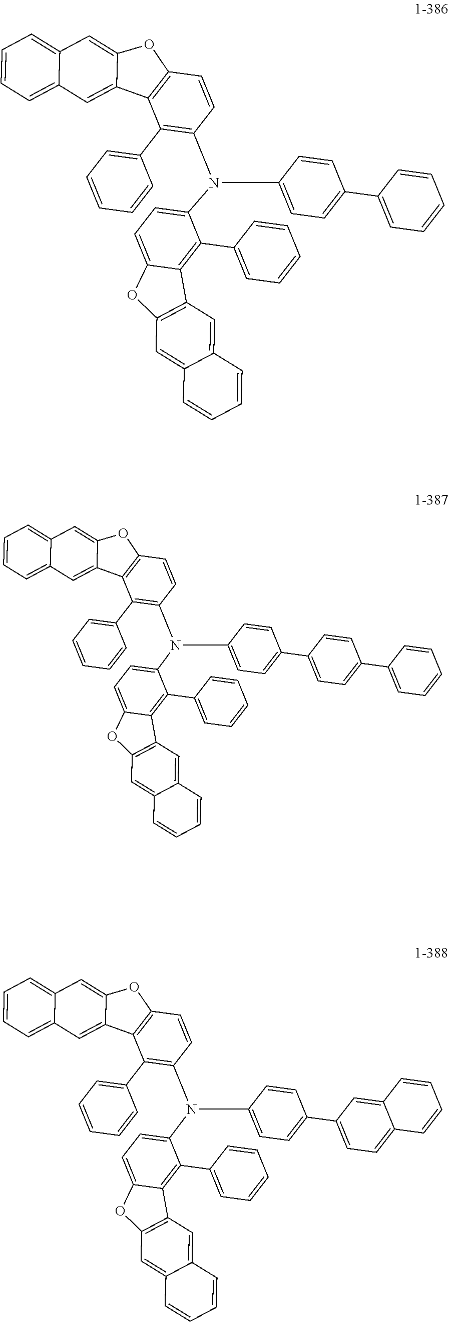 [SMILES: c1ccc(-c2ccc(N(c3ccc4oc5cc6ccccc6cc5c4c3-c3ccccc3)c3ccc4oc5cc6ccccc6cc5c4c3-c3ccccc3)cc2)cc1 ; c1ccc(-c2ccc(-c3ccc(N(c4ccc5oc6cc7ccccc7cc6c5c4-c4ccccc4)c4ccc5oc6cc7ccccc7cc6c5c4-c4ccccc4)cc3)cc2)cc1 ; c1ccc(-c2c(N(c3ccc(-c4ccc5ccccc5c4)cc3)c3ccc4oc5cc6ccccc6cc5c4c3-c3ccccc3)ccc3oc4cc5ccccc5cc4c23)cc1]

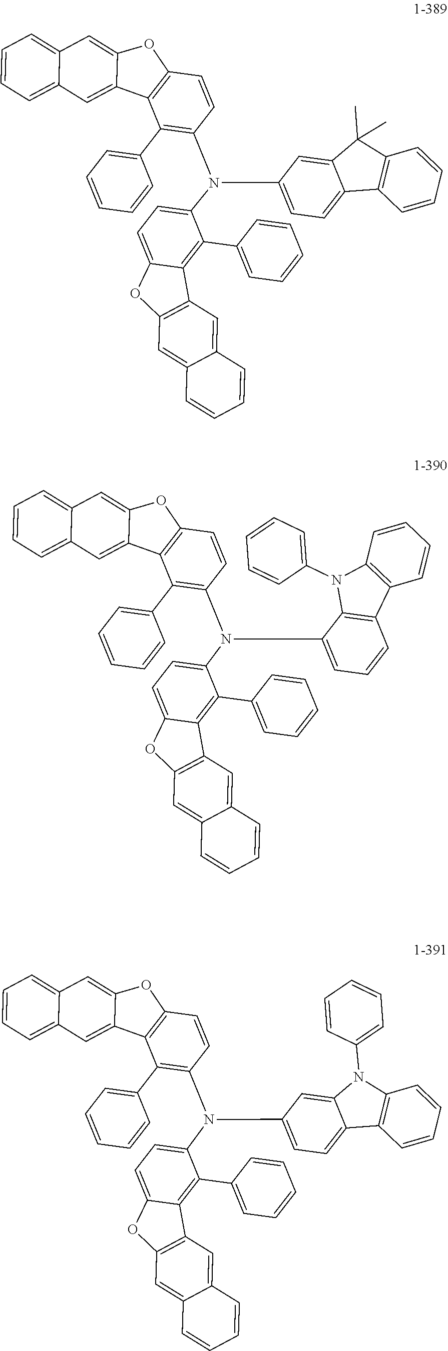 [SMILES: CC1(C)c2ccccc2-c2ccc(N(c3ccc4oc5cc6ccccc6cc5c4c3-c3ccccc3)c3ccc4oc5cc6ccccc6cc5c4c3-c3ccccc3)cc21 ; c1ccc(-c2c(N(c3ccc4oc5cc6ccccc6cc5c4c3-c3ccccc3)c3cccc4c5ccccc5n(-c5ccccc5)c34)ccc3oc4cc5ccccc5cc4c23)cc1 ; c1ccc(-c2c(N(c3ccc4c5ccccc5n(-c5ccccc5)c4c3)c3ccc4oc5cc6ccccc6cc5c4c3-c3ccccc3)ccc3oc4cc5ccccc5cc4c23)cc1]

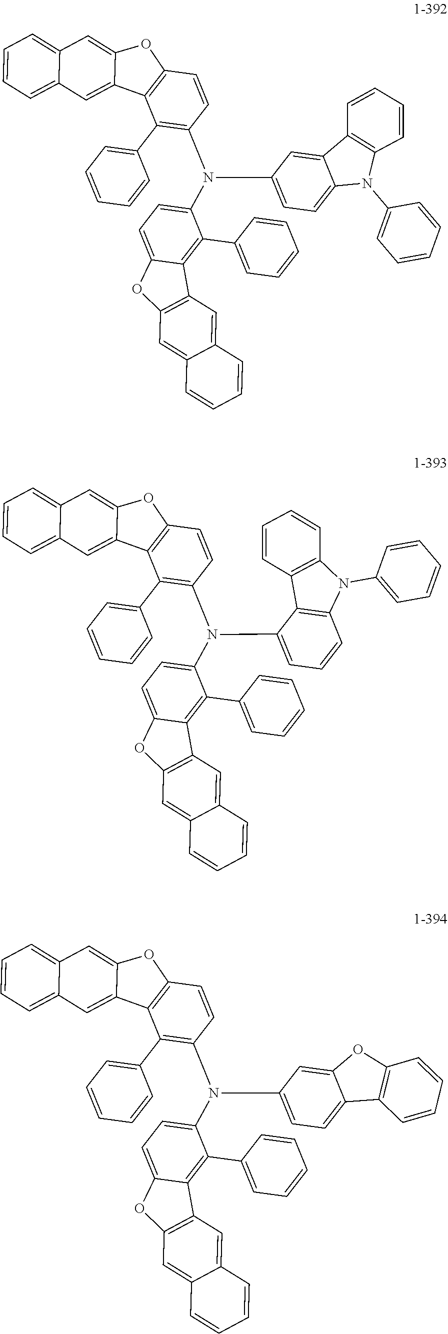 [SMILES: c1ccc(-c2c(N(c3ccc4c(c3)c3ccccc3n4-c3ccccc3)c3ccc4oc5cc6ccccc6cc5c4c3-c3ccccc3)ccc3oc4cc5ccccc5cc4c23)cc1 ; c1ccc(-c2c(N(c3ccc4oc5cc6ccccc6cc5c4c3-c3ccccc3)c3cccc4c3c3ccccc3n4-c3ccccc3)ccc3oc4cc5ccccc5cc4c23)cc1 ; c1ccc(-c2c(N(c3ccc4c(c3)oc3ccccc34)c3ccc4oc5cc6ccccc6cc5c4c3-c3ccccc3)ccc3oc4cc5ccccc5cc4c23)cc1]

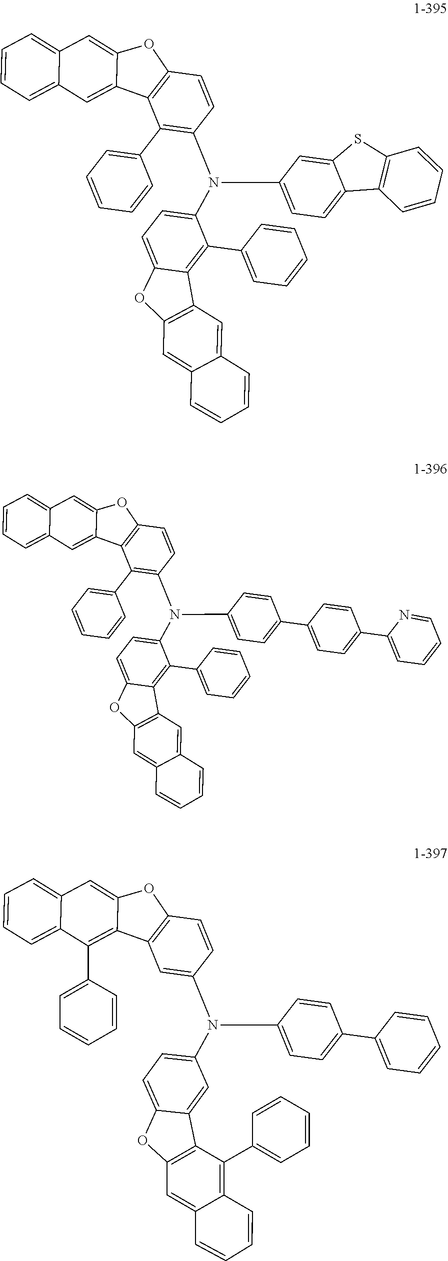 [SMILES: c1ccc(-c2c(N(c3ccc4c(c3)sc3ccccc34)c3ccc4oc5cc6ccccc6cc5c4c3-c3ccccc3)ccc3oc4cc5ccccc5cc4c23)cc1 ; c1ccc(-c2c(N(c3ccc(-c4ccc(-c5ccccn5)cc4)cc3)c3ccc4oc5cc6ccccc6cc5c4c3-c3ccccc3)ccc3oc4cc5ccccc5cc4c23)cc1 ; c1ccc(-c2ccc(N(c3ccc4oc5cc6ccccc6c(-c6ccccc6)c5c4c3)c3ccc4oc5cc6ccccc6c(-c6ccccc6)c5c4c3)cc2)cc1]

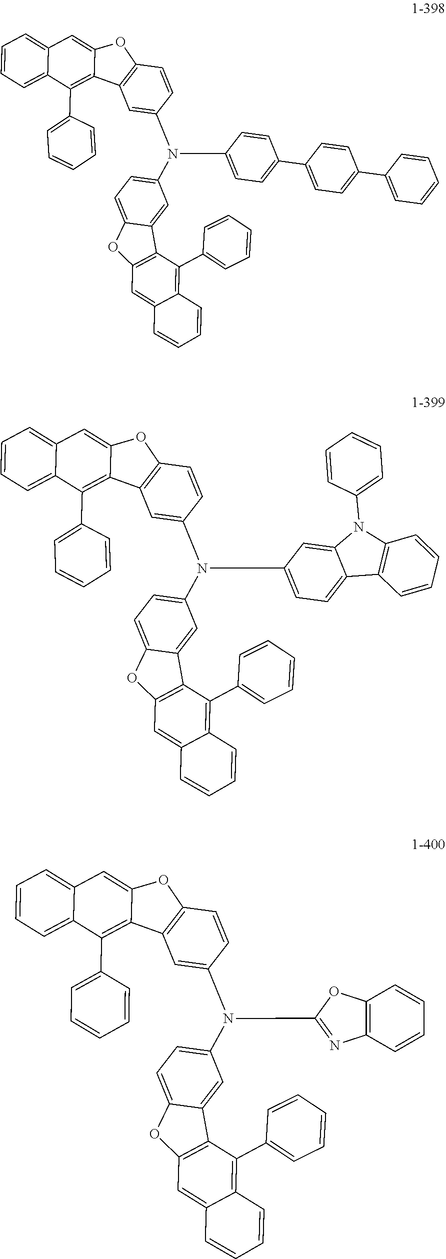 [SMILES: c1ccc(-c2ccc(-c3ccc(N(c4ccc5oc6cc7ccccc7c(-c7ccccc7)c6c5c4)c4ccc5oc6cc7ccccc7c(-c7ccccc7)c6c5c4)cc3)cc2)cc1 ; c1ccc(-c2c3ccccc3cc3oc4ccc(N(c5ccc6oc7cc8ccccc8c(-c8ccccc8)c7c6c5)c5ccc6c7ccccc7n(-c7ccccc7)c6c5)cc4c23)cc1 ; c1ccc(-c2c3ccccc3cc3oc4ccc(N(c5ccc6oc7cc8ccccc8c(-c8ccccc8)c7c6c5)c5nc6ccccc6o5)cc4c23)cc1]

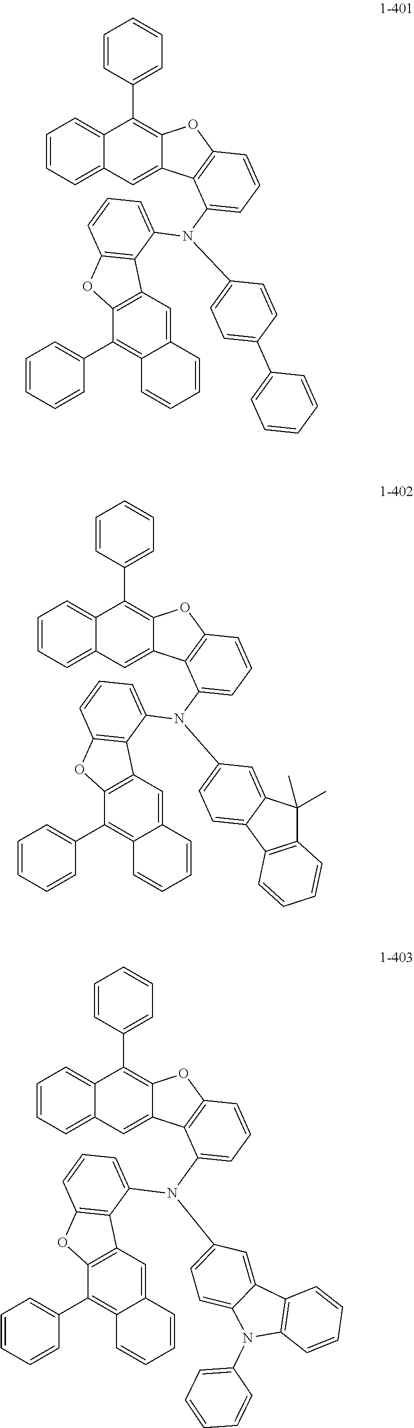 [SMILES: c1ccc(-c2ccc(N(c3cccc4oc5c(-c6ccccc6)c6ccccc6cc5c34)c3cccc4oc5c(-c6ccccc6)c6ccccc6cc5c34)cc2)cc1 ; CC1(C)c2ccccc2-c2ccc(N(c3cccc4oc5c(-c6ccccc6)c6ccccc6cc5c34)c3cccc4oc5c(-c6ccccc6)c6ccccc6cc5c34)cc21 ; c1ccc(-c2c3ccccc3cc3c2oc2cccc(N(c4ccc5c(c4)c4ccccc4n5-c4ccccc4)c4cccc5oc6c(-c7ccccc7)c7ccccc7cc6c45)c23)cc1]

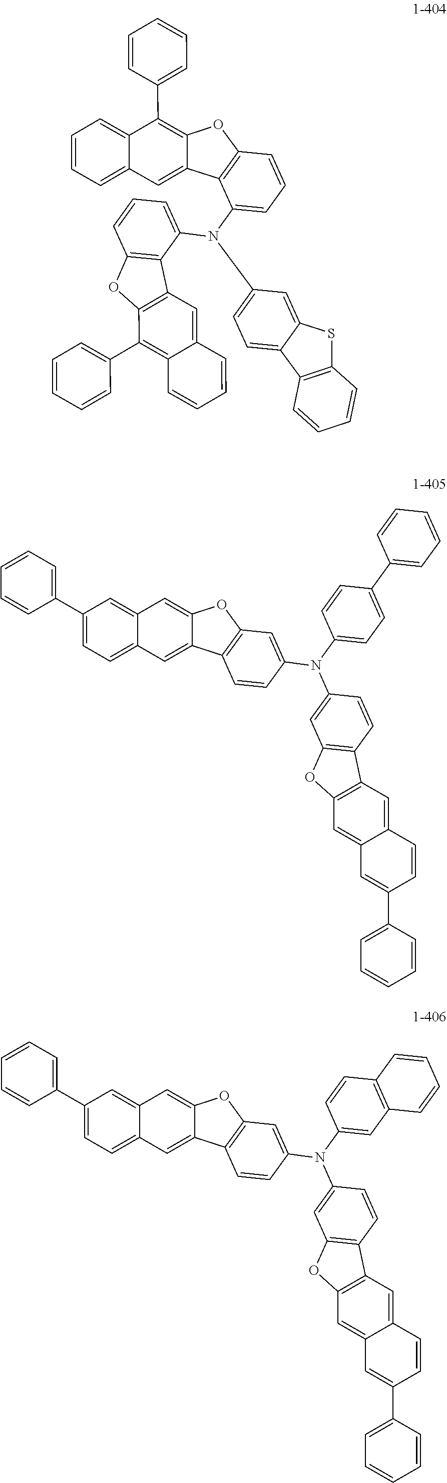 [SMILES: c1ccc(-c2c3ccccc3cc3c2oc2cccc(N(c4ccc5c(c4)sc4ccccc45)c4cccc5oc6c(-c7ccccc7)c7ccccc7cc6c45)c23)cc1 ; c1ccc(-c2ccc(N(c3ccc4c(c3)oc3cc5cc(-c6ccccc6)ccc5cc34)c3ccc4c(c3)oc3cc5cc(-c6ccccc6)ccc5cc34)cc2)cc1 ; c1ccc(-c2ccc3cc4c(cc3c2)oc2cc(N(c3ccc5ccccc5c3)c3ccc5c(c3)oc3cc6cc(-c7ccccc7)ccc6cc35)ccc24)cc1]

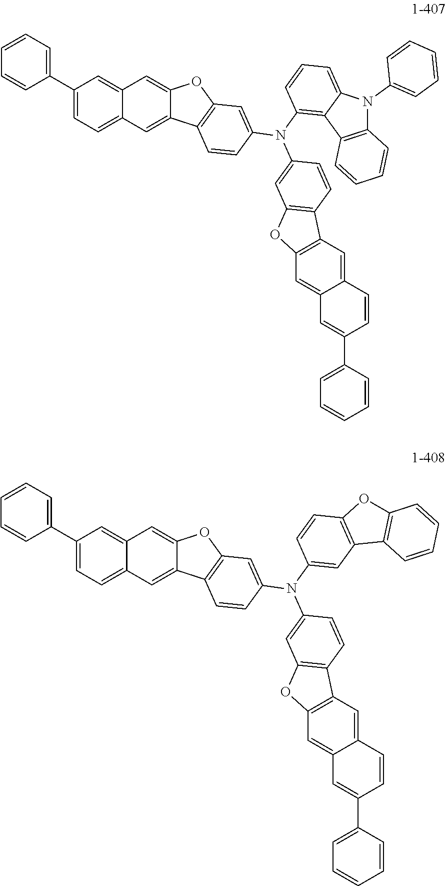 [SMILES: c1ccc(-c2ccc3cc4c(cc3c2)oc2cc(N(c3ccc5c(c3)oc3cc6cc(-c7ccccc7)ccc6cc35)c3cccc5c3c3ccccc3n5-c3ccccc3)ccc24)cc1 ; c1ccc(-c2ccc3cc4c(cc3c2)oc2cc(N(c3ccc5c(c3)oc3cc6cc(-c7ccccc7)ccc6cc35)c3ccc5oc6ccccc6c5c3)ccc24)cc1]

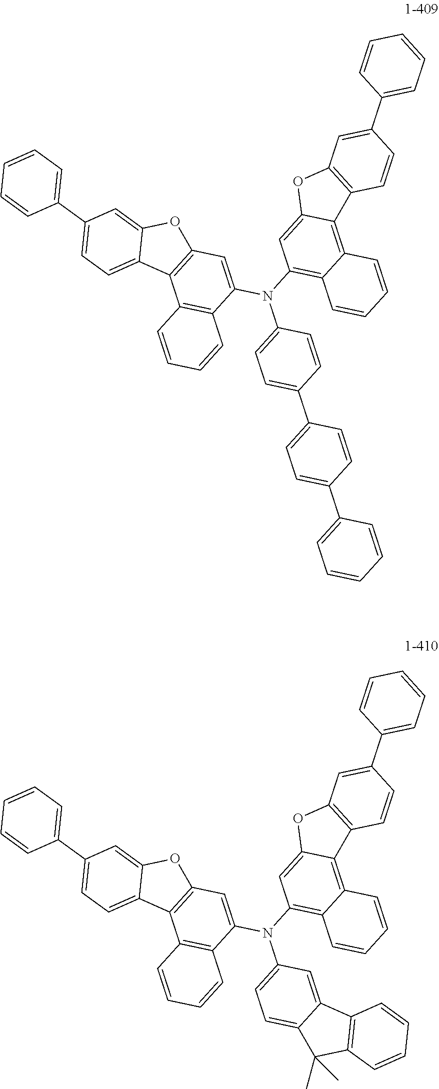 [SMILES: c1ccc(-c2ccc(-c3ccc(N(c4cc5oc6cc(-c7ccccc7)ccc6c5c5ccccc45)c4cc5oc6cc(-c7ccccc7)ccc6c5c5ccccc45)cc3)cc2)cc1 ; CC1(C)c2ccccc2-c2cc(N(c3cc4oc5cc(-c6ccccc6)ccc5c4c4ccccc34)c3cc4oc5cc(-c6ccccc6)ccc5c4c4ccccc34)ccc21]

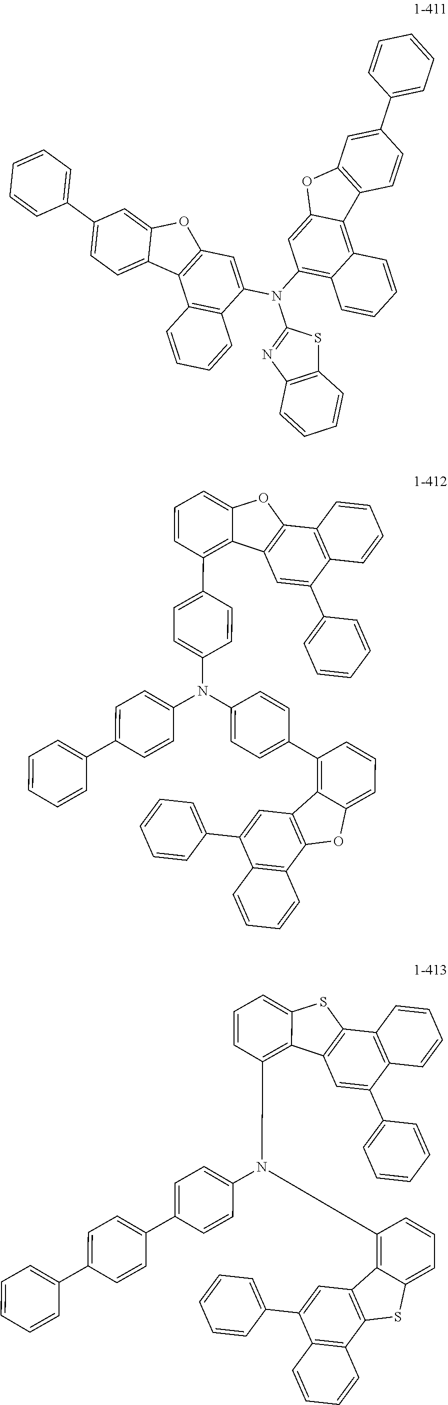 [SMILES: c1ccc(-c2ccc3c(c2)oc2cc(N(c4nc5ccccc5s4)c4cc5oc6cc(-c7ccccc7)ccc6c5c5ccccc45)c4ccccc4c23)cc1 ; c1ccc(-c2ccc(N(c3ccc(-c4cccc5oc6c7ccccc7c(-c7ccccc7)cc6c45)cc3)c3ccc(-c4cccc5oc6c7ccccc7c(-c7ccccc7)cc6c45)cc3)cc2)cc1 ; c1ccc(-c2ccc(-c3ccc(N(c4cccc5sc6c7ccccc7c(-c7ccccc7)cc6c45)c4cccc5sc6c7ccccc7c(-c7ccccc7)cc6c45)cc3)cc2)cc1]

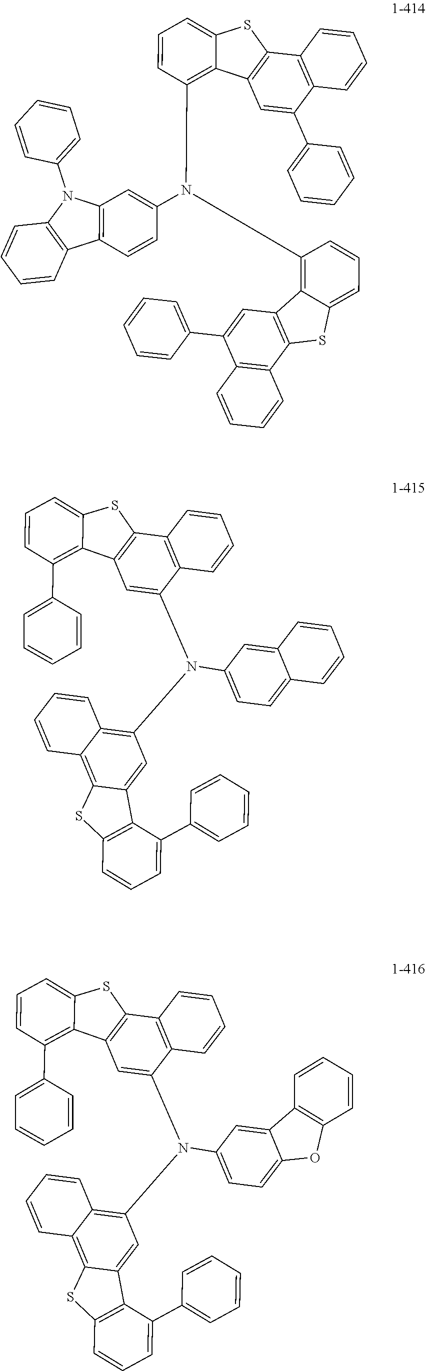 [SMILES: c1ccc(-c2cc3c(sc4cccc(N(c5ccc6c7ccccc7n(-c7ccccc7)c6c5)c5cccc6sc7c8ccccc8c(-c8ccccc8)cc7c56)c43)c3ccccc23)cc1 ; c1ccc(-c2cccc3sc4c5ccccc5c(N(c5ccc6ccccc6c5)c5cc6c(sc7cccc(-c8ccccc8)c76)c6ccccc56)cc4c23)cc1 ; c1ccc(-c2cccc3sc4c5ccccc5c(N(c5ccc6oc7ccccc7c6c5)c5cc6c(sc7cccc(-c8ccccc8)c76)c6ccccc56)cc4c23)cc1]

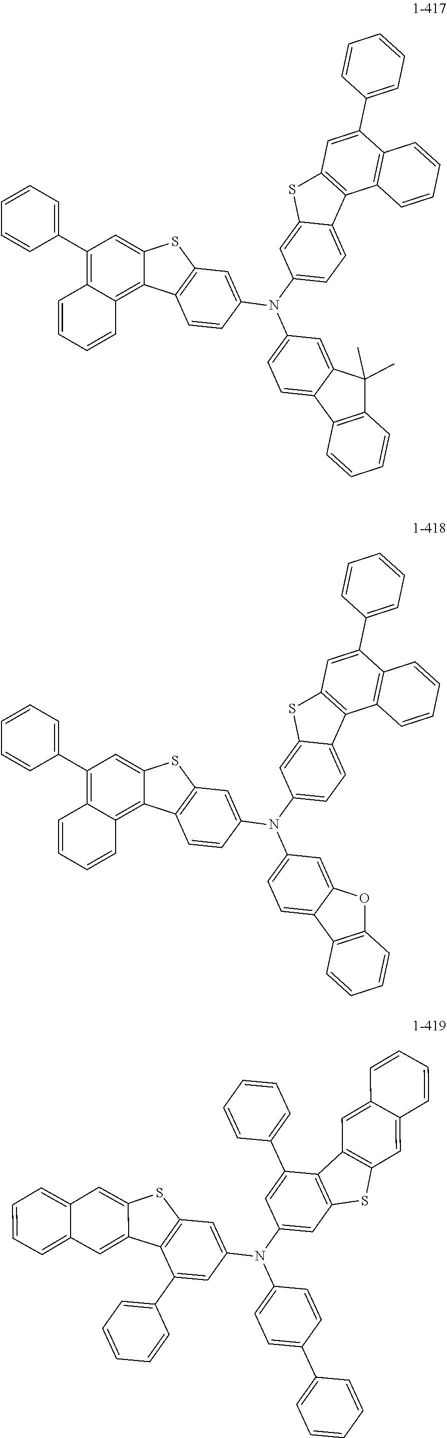 [SMILES: CC1(C)c2ccccc2-c2ccc(N(c3ccc4c(c3)sc3cc(-c5ccccc5)c5ccccc5c34)c3ccc4c(c3)sc3cc(-c5ccccc5)c5ccccc5c34)cc21 ; c1ccc(-c2cc3sc4cc(N(c5ccc6c(c5)oc5ccccc56)c5ccc6c(c5)sc5cc(-c7ccccc7)c7ccccc7c56)ccc4c3c3ccccc23)cc1 ; c1ccc(-c2ccc(N(c3cc(-c4ccccc4)c4c(c3)sc3cc5ccccc5cc34)c3cc(-c4ccccc4)c4c(c3)sc3cc5ccccc5cc34)cc2)cc1]

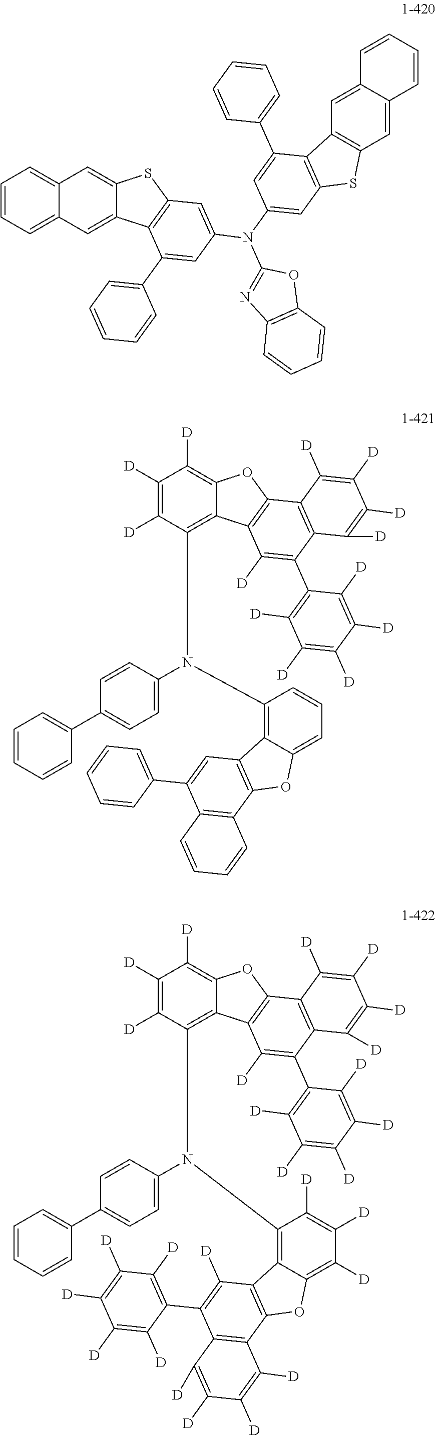

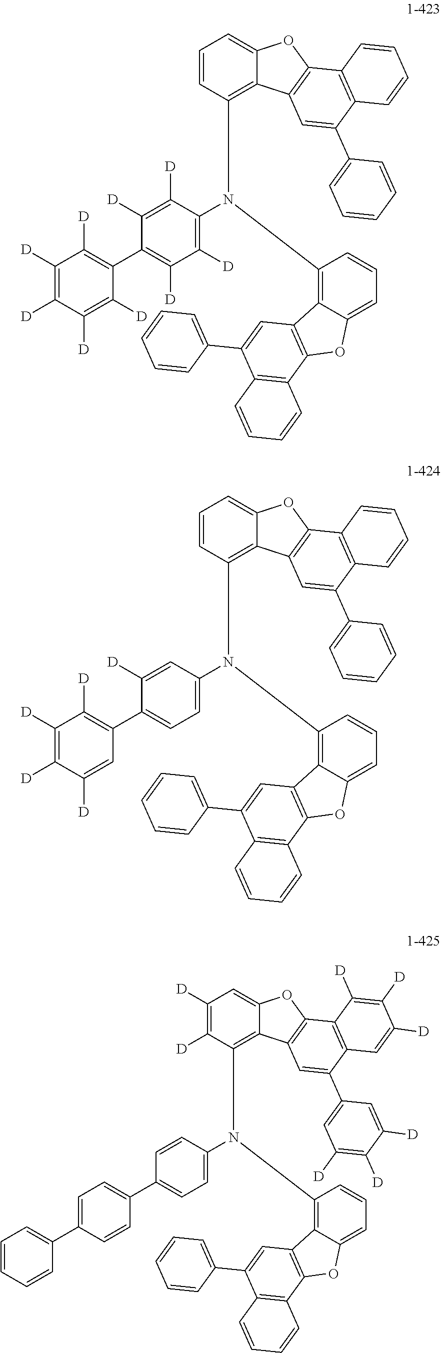 [SMILES: [2H]c1c([2H])c([2H])c(-c2c([2H])c([2H])c(N(c3cccc4oc5c6ccccc6c(-c6ccccc6)cc5c34)c3cccc4oc5c6ccccc6c(-c6ccccc6)cc5c34)c([2H])c2[2H])c([2H])c1[2H] ; [2H]c1cc(N(c2cccc3oc4c5ccccc5c(-c5ccccc5)cc4c23)c2cccc3oc4c5ccccc5c(-c5ccccc5)cc4c23)ccc1-c1cc([2H])c([2H])c([2H])c1[2H] ; [2H]c1cc(-c2cc3c(oc4cc([2H])c([2H])c(N(c5ccc(-c6ccc(-c7ccccc7)cc6)cc5)c5cccc6oc7c8ccccc8c(-c8ccccc8)cc7c56)c43)c3c([2H])c([2H])c([2H])cc23)cc([2H])c1[2H]]

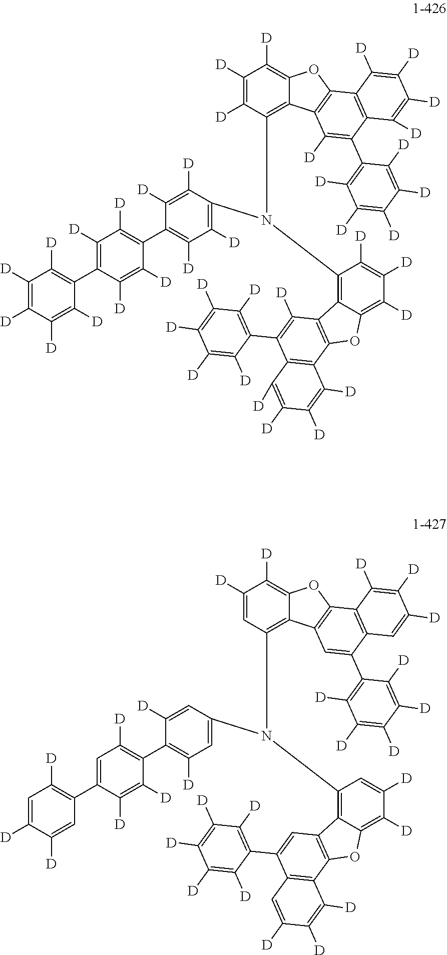

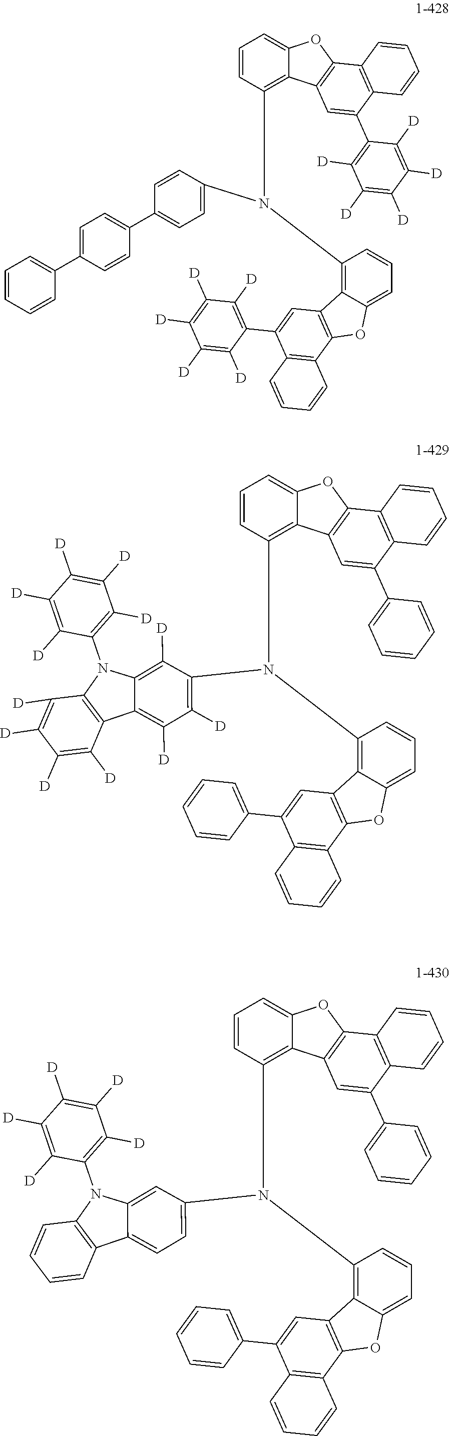 [SMILES: [2H]c1c([2H])c([2H])c(-c2cc3c(oc4cccc(N(c5ccc(-c6ccc(-c7ccccc7)cc6)cc5)c5cccc6oc7c8ccccc8c(-c8c([2H])c([2H])c([2H])c([2H])c8[2H])cc7c56)c43)c3ccccc23)c([2H])c1[2H] ; [2H]c1c([2H])c([2H])c(-n2c3c([2H])c([2H])c([2H])c([2H])c3c3c([2H])c([2H])c(N(c4cccc5oc6c7ccccc7c(-c7ccccc7)cc6c45)c4cccc5oc6c7ccccc7c(-c7ccccc7)cc6c45)c([2H])c32)c([2H])c1[2H] ; [2H]c1c([2H])c([2H])c(-n2c3ccccc3c3ccc(N(c4cccc5oc6c7ccccc7c(-c7ccccc7)cc6c45)c4cccc5oc6c7ccccc7c(-c7ccccc7)cc6c45)cc32)c([2H])c1[2H]]

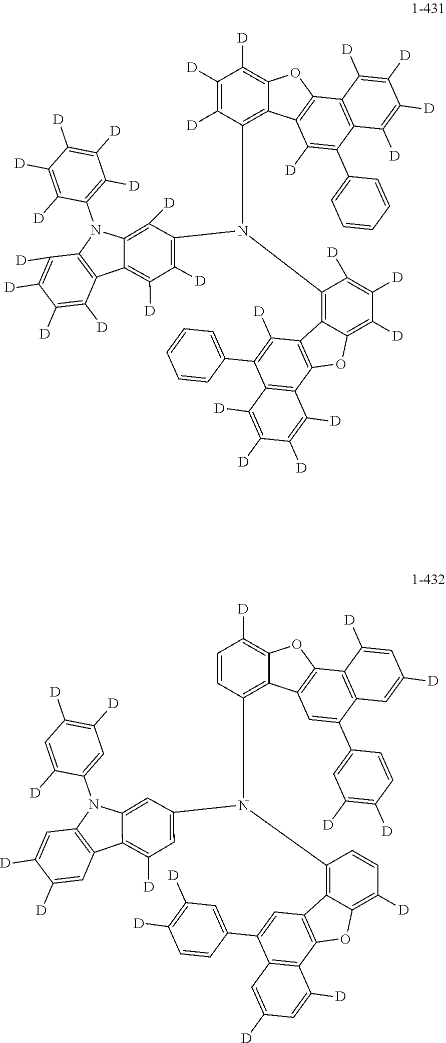 [SMILES: [2H]c1c([2H])c([2H])c(-n2c3c([2H])c([2H])c([2H])c([2H])c3c3c([2H])c([2H])c(N(c4c([2H])c([2H])c([2H])c5oc6c7c([2H])c([2H])c([2H])c([2H])c7c(-c7ccccc7)c([2H])c6c45)c4c([2H])c([2H])c([2H])c5oc6c7c([2H])c([2H])c([2H])c([2H])c7c(-c7ccccc7)c([2H])c6c45)c([2H])c32)c([2H])c1[2H] ; [2H]c1cc([2H])c2c(c1)c(-c1ccc([2H])c([2H])c1)cc1c2oc2c([2H])ccc(N(c3cc([2H])c4c5cc([2H])c([2H])cc5n(-c5cc([2H])c([2H])cc5[2H])c4c3)c3ccc([2H])c4oc5c(cc(-c6ccc([2H])c([2H])c6)c6cc([2H])cc([2H])c65)c34)c21]

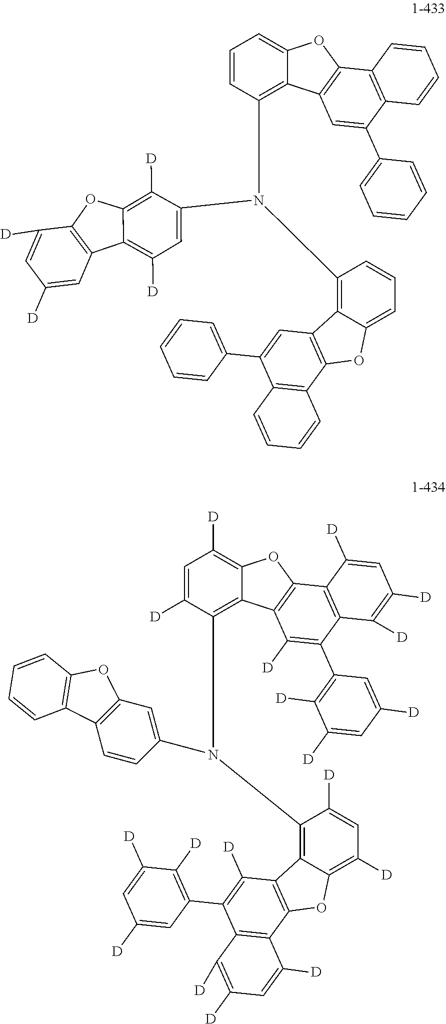 [SMILES: [2H]c1cc([2H])c2oc3c([2H])c(N(c4cccc5oc6c7ccccc7c(-c7ccccc7)cc6c45)c4cccc5oc6c7ccccc7c(-c7ccccc7)cc6c45)cc([2H])c3c2c1 ; [2H]c1cc([2H])c([2H])c(-c2c([2H])c3c(oc4c([2H])cc([2H])c(N(c5ccc6c(c5)oc5ccccc56)c5c([2H])cc([2H])c6oc7c8c([2H])cc([2H])c([2H])c8c(-c8cc([2H])cc([2H])c8[2H])c([2H])c7c56)c43)c3c([2H])cc([2H])c([2H])c23)c1]

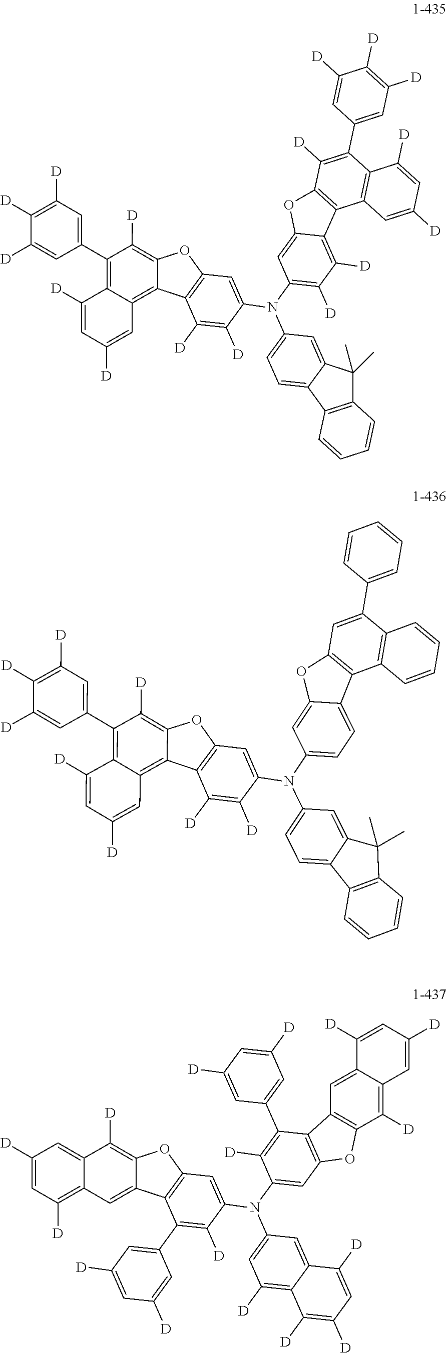

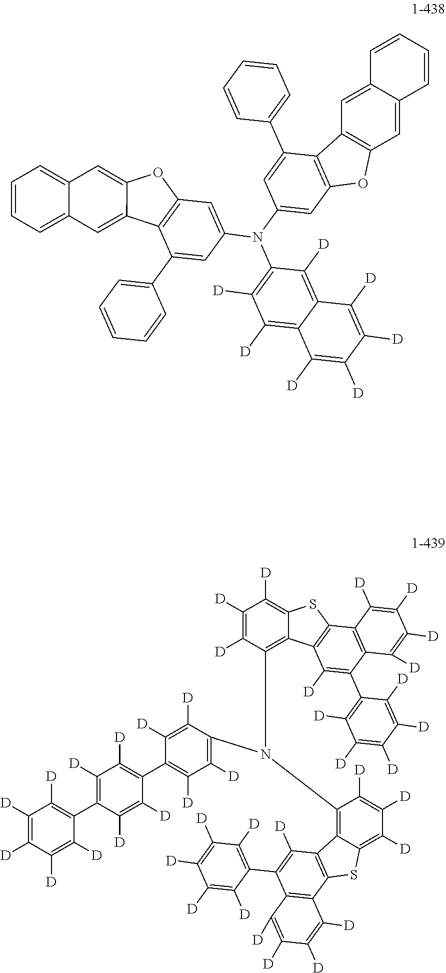 [SMILES: [2H]c1c([2H])c([2H])c2c([2H])c(N(c3cc(-c4ccccc4)c4c(c3)oc3cc5ccccc5cc34)c3cc(-c4ccccc4)c4c(c3)oc3cc5ccccc5cc34)c([2H])c([2H])c2c1[2H] ; [2H]c1c([2H])c([2H])c(-c2c([2H])c([2H])c(-c3c([2H])c([2H])c(N(c4c([2H])c([2H])c([2H])c5sc6c7c([2H])c([2H])c([2H])c([2H])c7c(-c7c([2H])c([2H])c([2H])c([2H])c7[2H])c([2H])c6c45)c4c([2H])c([2H])c([2H])c5sc6c7c([2H])c([2H])c([2H])c([2H])c7c(-c7c([2H])c([2H])c([2H])c([2H])c7[2H])c([2H])c6c45)c([2H])c3[2H])c([2H])c2[2H])c([2H])c1[2H]]

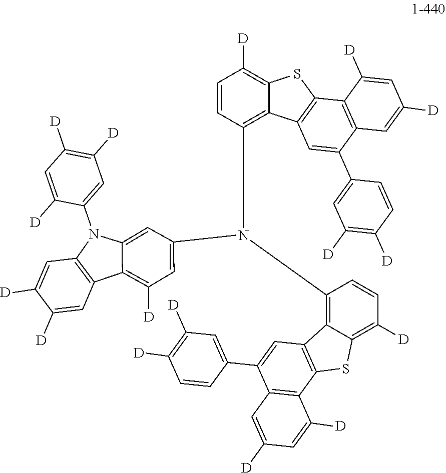 [SMILES: [2H]c1cc([2H])c2c(c1)c(-c1ccc([2H])c([2H])c1)cc1c2sc2c([2H])ccc(N(c3cc([2H])c4c5cc([2H])c([2H])cc5n(-c5cc([2H])c([2H])cc5[2H])c4c3)c3ccc([2H])c4sc5c(cc(-c6ccc([2H])c([2H])c6)c6cc([2H])cc([2H])c65)c34)c21]

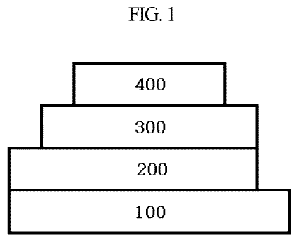

FIGS. 1 to 3 illustrate the stacking order of electrodes and organic layers of a light-emitting device according to an embodiment of the present application. However, the scope of the present application is not intended to be limited to these drawings, and the structure of organic light-emitting devices known in the art may also be applied to the present application.

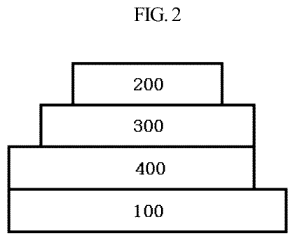

Referring to FIG. 1, a light-emitting device is illustrated, in which a first electrode (anode, 200), an organic layer 300, and a second electrode (cathode, 400) are sequentially stacked on a substrate 100. However, it is not limited to this structure, and as shown in FIG. 2, an organic light-emitting device may be implemented in which the cathode 400, the organic layer 300, and the anode 200 are sequentially stacked on the substrate.

In an exemplary embodiment, the first electrode may be an anode, and the second electrode may be a cathode. Alternatively, the first electrode may be a cathode, and the second electrode may be an anode.

According to exemplary embodiments, materials with a relatively high work function may be used as the anode material, such as transparent conductive oxides, metals, or conductive polymers. Examples of the anode material may include metals such as vanadium, chromium, copper, zinc, and gold, or an alloy thereof, metal oxides such as zinc oxide, indium oxide, indium tin oxide (ITO), and indium zinc oxide (IZO); combinations of metals and oxides such as ZnO:Al or SnO₂:Sb; and conductive polymers such as poly(3-methylthiophene), poly[3,4-(ethylene-1,2-dioxy)thiophene](PEDOT), polypyrrole and polyaniline, but are not limited thereto.

Materials with a relatively low work function may be used as cathode materials, such as metals, metal oxides, or conductive polymers. Examples of cathode materials may include metals such as magnesium, calcium, sodium, potassium, titanium, indium, yttrium, lithium, gadolinium, aluminum, silver, tin, and lead, or their alloys; and multilayer materials such as LiF/Al or LiO₂/Al, but are not limited thereto.

The light-emitting device may include one or more organic layers 300. Each of the plurality of organic layers 300 may independently have a single-layer or multilayer structure.

In an exemplary embodiment, at least one of the organic layers 300 may include a heterocyclic compound represented by Formula 1 above.

In an exemplary embodiment, at least one of the organic layers 300 may include two or more heterocyclic compounds represented by Formula 1 above.

As used herein, the specific details of the heterocyclic compound represented by Formula 1 are the same as those described above.

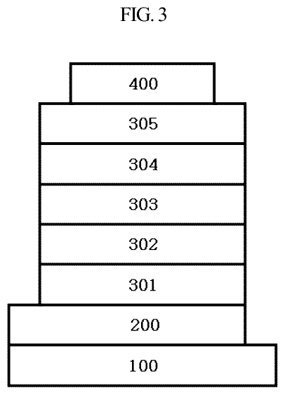

FIG. 3 is a cross-sectional view illustrating an exemplary light-emitting device in which the organic layer has a multilayer structure.

The light-emitting device shown in FIG. 3 may include a hole injection layer 301, a hole transport layer 302, a light-emitting layer 303, an electron transport layer 304, and an electron injection layer 305. However, the scope of the present application is not limited to this stacked structure, and layers other than the light-emitting layer may be omitted, and other functional layers may be added.

The light-emitting layer 303 may include a host material that is excited by holes and electrons, and a dopant material that increases light-emitting efficiency through energy absorption and emission.

In one embodiment, the light-emitting layer 303 may be independently patterned for each red light-emitting device (Pr), green light-emitting device (Pg), and blue light-emitting device (Pb), thereby emitting different colors of light for each element. For example, the light-emitting layer 303 may be patterned into a red light-emitting layer, a greenlight-emitting layer, and a blue light-emitting layer for each element.

In one embodiment, the light-emitting layer 303 may not be patterned for each light-emitting device, but may be provided in common to a plurality of light-emitting devices. For example, the light-emitting layer 303 may emit white light, and the color of each element may be implemented through a color filter.

The host material may include a phosphorescent host, a fluorescent host, or a combination thereof. For example, the host material may include ADN (9,10-di(2-naphthyl)anthracene), MADN (2-methyl-9,10-bis(naphthalen-2-yl)anthracene), TBADN (9,10-di-(2-naphthyl)-2-t-butyl-anthracene), CBP (4,4′-bis(N-carbazolyl)-1,1′-biphenyl), mCP (1,3-di-9-caibazolylbenzene), TCP (1,3,5-tri(carbazol-9-yl)benzene), DPEPO (bis[2-(diphenylphosphino)phenyl]ether oxide), PPF (2,8-bis(diphenylphosphoryl)dibenzo[b,d]furan), TCTA (4,4′,4″-tris(catbazol-9-yl)-triphenylamine), CP1 (hexaphenylcyclotriphosphazene), UGH2 (1,4-bis(triphenylsilyl)benzene), DPSiO₃ (hexaphenylcyclotrisiloxane), DPSiO₄(octaphenylcyclotetrasiloxane), or a combination thereof.

The dopant material may include a phosphorescent dopant, a fluorescent dopant, or a combination thereof. For example, the dopant material may include a metal complex containing iridium (Ir), platinum (Pt), osmium (Os), gold (Au), titanium (Ti), zirconium (Zr), hafnium (Hf), europium (Eu), terbium (Tb), or thulium (Tm), or BCzVB (1,4-bis[2-(3-N-ethylcarbazolyl)vinyl]benzene), DPAVB (4-(di-p-tolylamino)-4′-[(di-p-tolylamino)styryl]stilbene), N-BDAVBi (N-(4-((E)-2-(6-((E)-4-(diphenylamino)styryl)naphthalen-2-yl)vinyl)phenyl)-N-phenylbenzenamine), DPAVBi (4,4′-bis[2-(4-(N,N-diphenylamino)phenyl)vinyl]biphenyl), TBP (2,5,8,11-tetra-t-butylperylene), or a combination thereof.

In one embodiment; the host material may include a heterocyclic compound represented by Formula 1 above. For example, the heterocyclic compound may serve as a red phosphorescent dopant.

By including the heterocyclic compound as the host material, the charge transport properties within the light-emitting device and the stability of the light-emitting layer 303 may be further improved, thereby increasing the light-emitting efficiency and lifetime of the light-emitting device. Accordingly, the light-emitting properties may be improved without increasing the operating voltage.

In some embodiments, the content of the dopant material in the light-emitting layer 303 may be about 0.01 parts by weight or more, about 1 part by weight or more, or about 2 parts by weight or more, and may be about 15 parts by weight or less, about 10 parts by weight or less, or about 8 parts by weight or less, based on 100 parts by weight of the host material. Within this range, the formation and emission energy of excitons may be increased, and the light-emitting efficiency and stability may be further improved.

In some embodiments, the organic layer 300 may further include a compound represented by Formula 3 below.

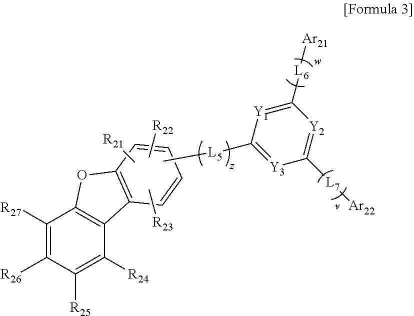

In Formula 3, any one of R₂₁ to R₂₇ may be a substituted or unsubstituted aryl group having 6 to 60 carbon atoms, or a substituted or unsubstituted heteroaryl group having 2 to 60 carbon atoms, and the others of R₂₁ to R₂₇ may each independently be selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a phosphine oxide group; and a silyl group; or two or more adjacent groups may be linked to form a substituted or unsubstituted aromatic hydrocarbon ring having 6 to 60 carbon atoms, or a substituted or unsubstituted heterocycle having 2 to 60 carbon atoms.

Preferably, when any one of R₂₁ to R₂₇ is an aryl group or a heteroaryl group, the number of carbon atoms may be 50 or less, 40 or less, 30 or less, or 20 or less, and more preferably 12 or less.

L₅, L₆, and L₇ are each independently a direct linkage, a substituted or unsubstituted arylene group having 6 to 60 carbon atoms, or a substituted or unsubstituted heteroarylene group having 2 to 60 carbon atoms.

In some embodiments, when L₅, L₆, and L₇ are each independently an arylene group or a heteroarylene group, the number of carbon atoms may be 50 or less, 40 or less, 30 or less, or 20 or less, and preferably 12 or less.

For example, L₅ to L₇ are each independently a direct linkage, or a divalent group obtained by removing one hydrogen atom from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylenyl group, a substituted or unsubstituted anthryl group, a substituted or unsubstituted dimethylfluorenyl group, a substituted or unsubstituted diethylfluorenyl group, a substituted or unsubstituted spirobifluorenyl group, a substituted or unsubstituted furan group, a substituted or unsubstituted thiophene group, a substituted or unsubstituted benzofuran group, a substituted or unsubstituted benzothiophene group, a substituted or unsubstituted dibenzofuran group, a substituted or unsubstituted dibenzothiophene group, or a substituted or unsubstituted carbazolyl group.

For example, L₅ to L₇ may each independently be a direct linkage, or a divalent group obtained by removing one hydrogen atom from a deuterated or non-deuterated phenyl group, a deuterated or non-deuterated biphenyl group, a deuterated or non-deuterated terphenyl group, a deuterated or non-deuterated naphthyl group, a deuterated or non-deuterated phenanthrenyl group, a deuterated or non-deuterated triphenylenyl group, a deuterated or non-deuterated dimethylfluorenyl group, a deuterated or non-deuterated diethylfluorenyl group, a deuterated or non-deuterated spirobifluorenyl group, a deuterated or non-deuterated furan group, a deuterated or non-deuterated thiophene group, a deuterated or non-deuterated benzofuran group, a deuterated or non-deuterated benzothiophene group, a deuterated or non-deuterated dibenzofuran group, a deuterated or non-deuterated dibenzothiophene group, a deuterated or non-deuterated anthryl group, a deuterated or non-deuterated carbazolyl group.

In Formula 3, w, v, and z are each independently 0 or an integer of 1 to 5. For example, w, v, and z may each independently be 0, 1, or 2.

In Formula 3, when w, v, and z are integers of 2 to 5, a plurality of L₅ groups, L₆ groups, and L₇ groups may each be the same or different.

Y₁ to Y₃ may each independently be CH, nitrogen (N), oxygen (O), or sulfur (S), and at least two of Y₁ to Y₃ may be nitrogen. Preferably, Y₁ to Y₃ are all nitrogen.

Ar₂₁ and Ar₂₂ are each independently a substituted or unsubstituted aryl group having 6 to 60 carbon atoms, a substituted or unsubstituted heteroaryl group having 2 to 60 carbon atoms, or a substituted or unsubstituted nitrogen-containing heterocyclic ring having 2 to 60 carbon atoms.

Preferably, the number of carbon atoms of Ar₂₁ and Ar₂₂ is each 50 or less, 40 or less, 30 or less, or 20 or less, and more preferably 12 or less.

Adjacent groups among R₂₄ to R₂₇ may be linked to form an aromatic ring. For example, the compound represented by Formula 3 may include compounds represented by Formulae 3-1 to 3-6 below.

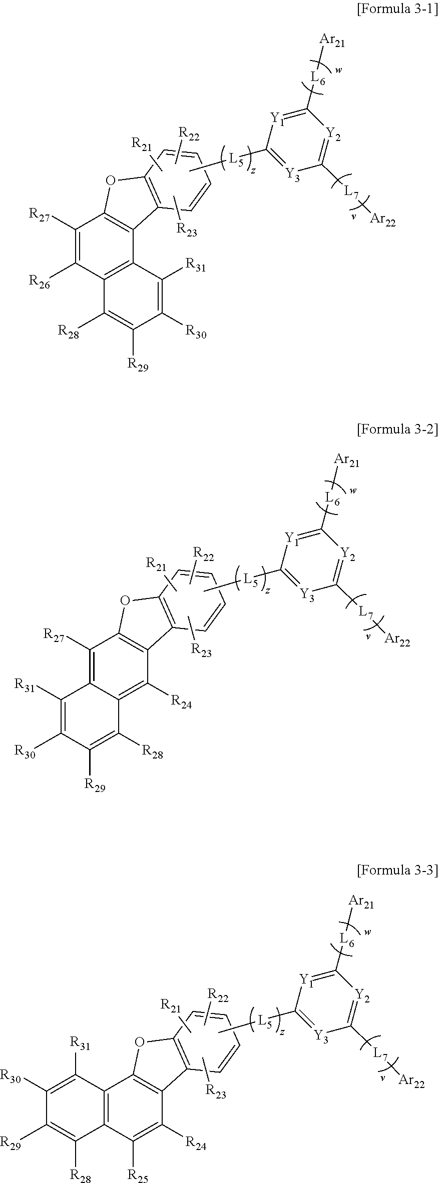

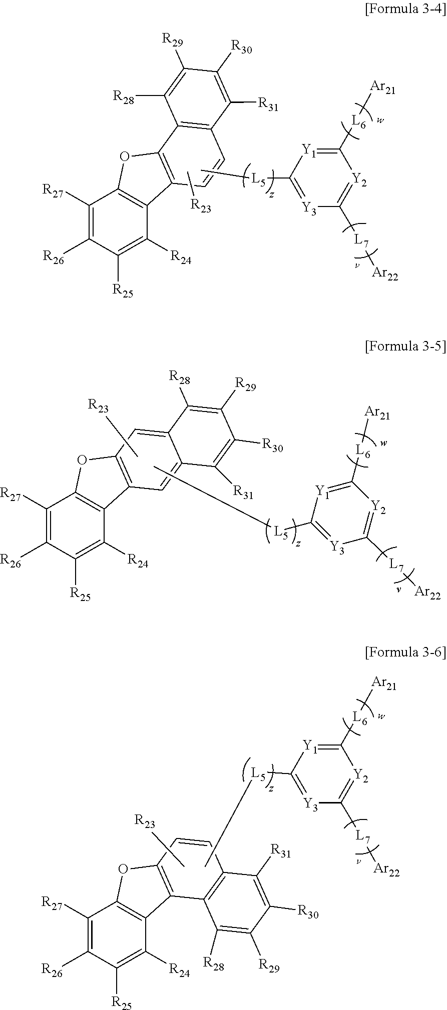

In Formulae 3-1 to 3-6, R₂₁ to R₂₇, Y₁ to Y₃, Ar₂₁, Ar₂₂, L₄ to L₇, z, w, and v may be the same as defined in Formula 3.

In Formulae 3-1 to 3-6, R₂₈ to R₃₁ may each independently be selected from the group consisting of hydrogen; deuterium; a halogen; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; —SiRR′R″; and —P(═O)RR′, wherein adjacent groups may be linked to form a substituted or unsubstituted 5- to 9-membered saturated or unsaturated ring.

R, R′, and R″ may each independently be hydrogen; deuterium; —CN; a substituted or unsubstituted alkyl group having 1 to 60 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms.

In Formula 3-1, R₂₆ and R₂₈ may be linked to each other to form a substituted or unsubstituted aromatic hydrocarbon ring having 6 to 60 carbon atoms, or a substituted or unsubstituted heterocycle having 2 to 60 carbon atoms.

In Formula 3-2, R₂₇ and R₃₁ may be linked to each other to form a substituted or unsubstituted aromatic hydrocarbon ring having 6 to 60 carbon atoms, or a substituted or unsubstituted heterocycle having 2 to 60 carbon atoms, and R₂₄ and R₂₈ may likewise be linked to each other to form a substituted or unsubstituted aromatic hydrocarbon ring having 6 to 60 carbon atoms, or a substituted or unsubstituted heterocycle having 2 to 60 carbon atoms.

In Formula 3-3, R₂₅ and R₂₈ may be linked to each other to form a substituted or unsubstituted aromatic hydrocarbon ring having 6 to 60 carbon atoms, or a substituted or unsubstituted heterocyclic ring having 2 to 60 carbon atoms.

In exemplary embodiments, the deuterium content of the compound of Formula 3 may be 0%, or greater than 0 and not more than 100%.

In exemplary embodiments, the deuterium content of the compound of Formula 3 may be 0%, or 5% to 100%.

According to exemplary embodiments, the deuterium content of the compound of Formula 3 may be 0%, or 10% to 100%.

According to exemplary embodiments, the deuterium content of the compound of Formula 3 may be 0%, or 15% to 100%.

According to exemplary embodiments, the deuterium content of the compound of Formula 3 may be 0%, or 20% to 100%.

According to exemplary embodiments, the deuterium content of the compound of Formula 3 may be 0%, or 25% to 100%.

According to exemplary embodiments, the deuterium content of the compound of Formula 3 may be 0%, or 30% to 100%.

According to exemplary embodiments, the deuterium content of the compound of Formula 3 may be 0%, or 50% to 100%.

According to exemplary embodiments, the deuterium content of the compound of Formula 3 may be 0%, or may be 70% to 100%.

According to exemplary embodiments, the deuterium content of the compound of Formula 3 may be 0%, or may be 90% to 100%.

According to exemplary embodiments, the deuterium content of the compound of Formula 3 may be 0%.

According to exemplary embodiments, the deuterium content of the compound of Formula 3 may be 30% to 100%.

According to exemplary embodiments, the deuterium content of the compound of Formula 3 may be 50% to 100%.

According to exemplary embodiments, the deuterium content of the compound of Formula 3 may be 70% to 100%.

According to exemplary embodiments, the deuterium content of the compound of Formula 3 may be 90% to 100%.

The compound of Formula 3 may be represented by any one of formulae below:

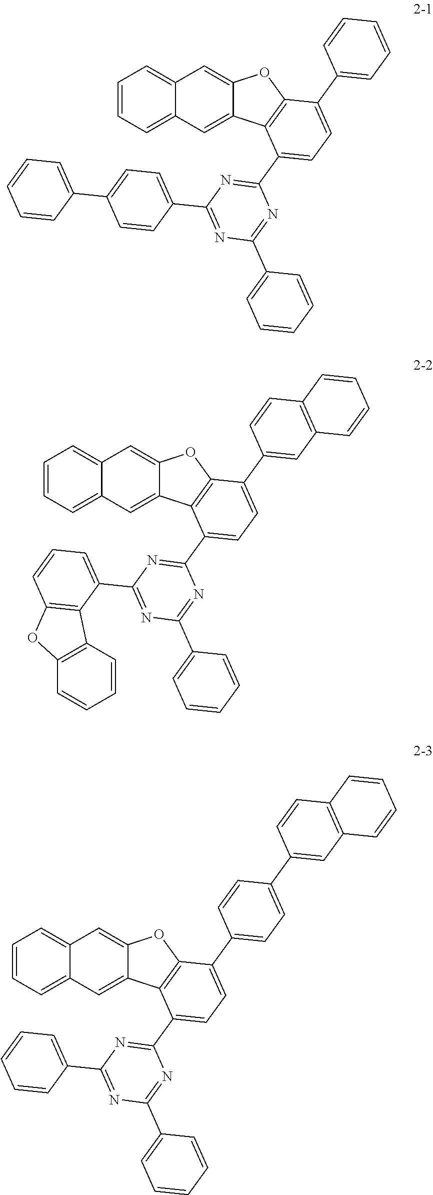 [SMILES: c1ccc(-c2ccc(-c3nc(-c4ccccc4)nc(-c4ccc(-c5ccccc5)c5oc6cc7ccccc7cc6c45)n3)cc2)cc1 ; c1ccc(-c2nc(-c3cccc4oc5ccccc5c34)nc(-c3ccc(-c4ccc5ccccc5c4)c4oc5cc6ccccc6cc5c34)n2)cc1 ; c1ccc(-c2nc(-c3ccccc3)nc(-c3ccc(-c4ccc(-c5ccc6ccccc6c5)cc4)c4oc5cc6ccccc6cc5c34)n2)cc1]

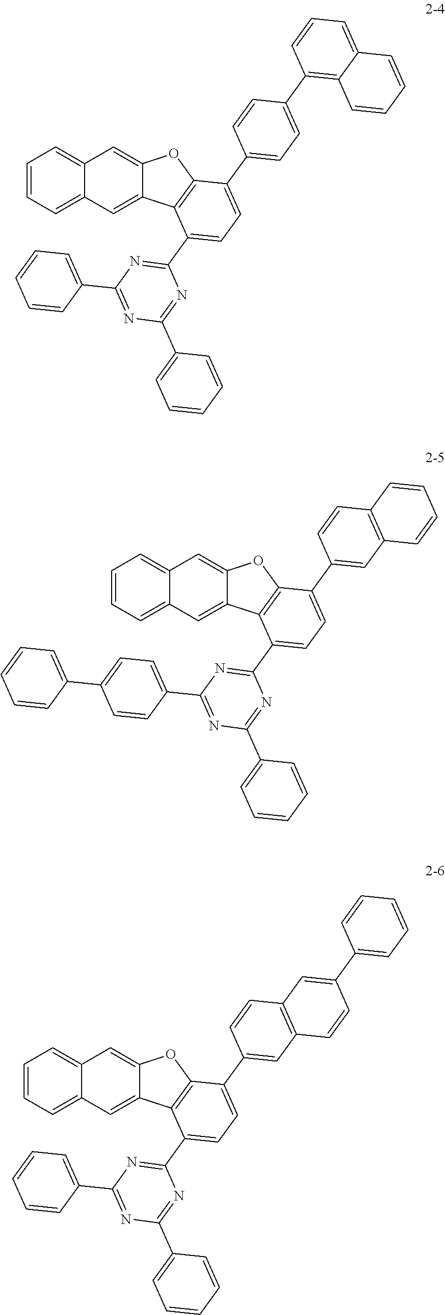 [SMILES: c1ccc(-c2nc(-c3ccccc3)nc(-c3ccc(-c4ccc(-c5cccc6ccccc56)cc4)c4oc5cc6ccccc6cc5c34)n2)cc1 ; c1ccc(-c2ccc(-c3nc(-c4ccccc4)nc(-c4ccc(-c5ccc6ccccc6c5)c5oc6cc7ccccc7cc6c45)n3)cc2)cc1 ; c1ccc(-c2ccc3cc(-c4ccc(-c5nc(-c6ccccc6)nc(-c6ccccc6)n5)c5c4oc4cc6ccccc6cc45)ccc3c2)cc1]

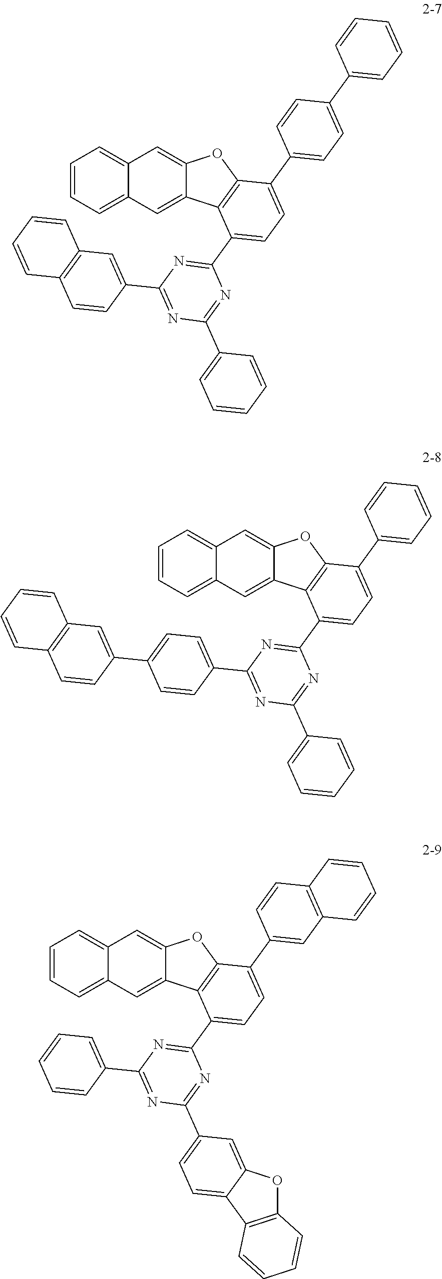 [SMILES: c1ccc(-c2ccc(-c3ccc(-c4nc(-c5ccccc5)nc(-c5ccc6ccccc6c5)n4)c4c3oc3cc5ccccc5cc34)cc2)cc1 ; c1ccc(-c2nc(-c3ccc(-c4ccc5ccccc5c4)cc3)nc(-c3ccc(-c4ccccc4)c4oc5cc6ccccc6cc5c34)n2)cc1 ; c1ccc(-c2nc(-c3ccc4c(c3)oc3ccccc34)nc(-c3ccc(-c4ccc5ccccc5c4)c4oc5cc6ccccc6cc5c34)n2)cc1]

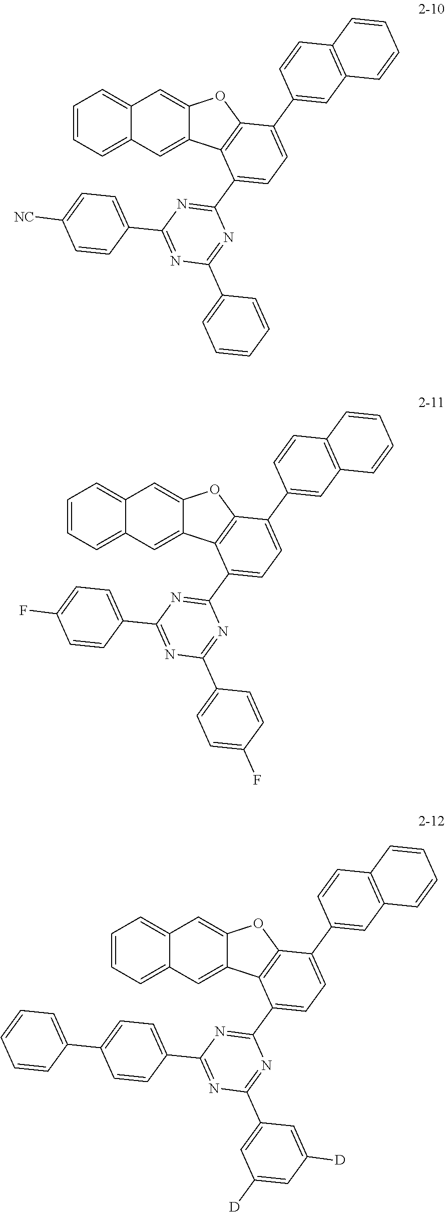 [SMILES: N#Cc1ccc(-c2nc(-c3ccccc3)nc(-c3ccc(-c4ccc5ccccc5c4)c4oc5cc6ccccc6cc5c34)n2)cc1 ; Fc1ccc(-c2nc(-c3ccc(F)cc3)nc(-c3ccc(-c4ccc5ccccc5c4)c4oc5cc6ccccc6cc5c34)n2)cc1 ; [2H]c1cc([2H])cc(-c2nc(-c3ccc(-c4ccccc4)cc3)nc(-c3ccc(-c4ccc5ccccc5c4)c4oc5cc6ccccc6cc5c34)n2)c1]

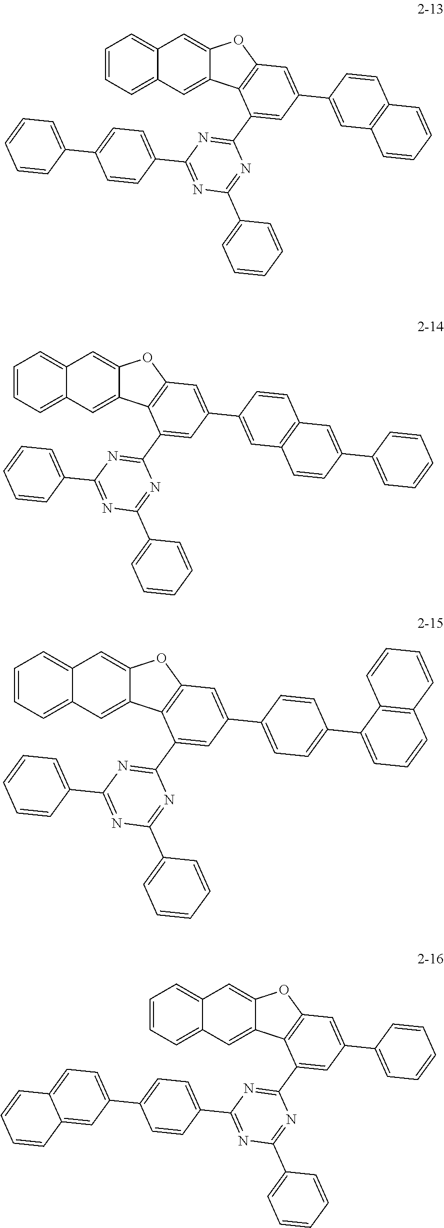 [SMILES: c1ccc(-c2ccc(-c3nc(-c4ccccc4)nc(-c4cc(-c5ccc6ccccc6c5)cc5oc6cc7ccccc7cc6c45)n3)cc2)cc1 ; c1ccc(-c2ccc3cc(-c4cc(-c5nc(-c6ccccc6)nc(-c6ccccc6)n5)c5c(c4)oc4cc6ccccc6cc45)ccc3c2)cc1 ; c1ccc(-c2nc(-c3ccccc3)nc(-c3cc(-c4ccc(-c5cccc6ccccc56)cc4)cc4oc5cc6ccccc6cc5c34)n2)cc1 ; c1ccc(-c2cc(-c3nc(-c4ccccc4)nc(-c4ccc(-c5ccc6ccccc6c5)cc4)n3)c3c(c2)oc2cc4ccccc4cc23)cc1]

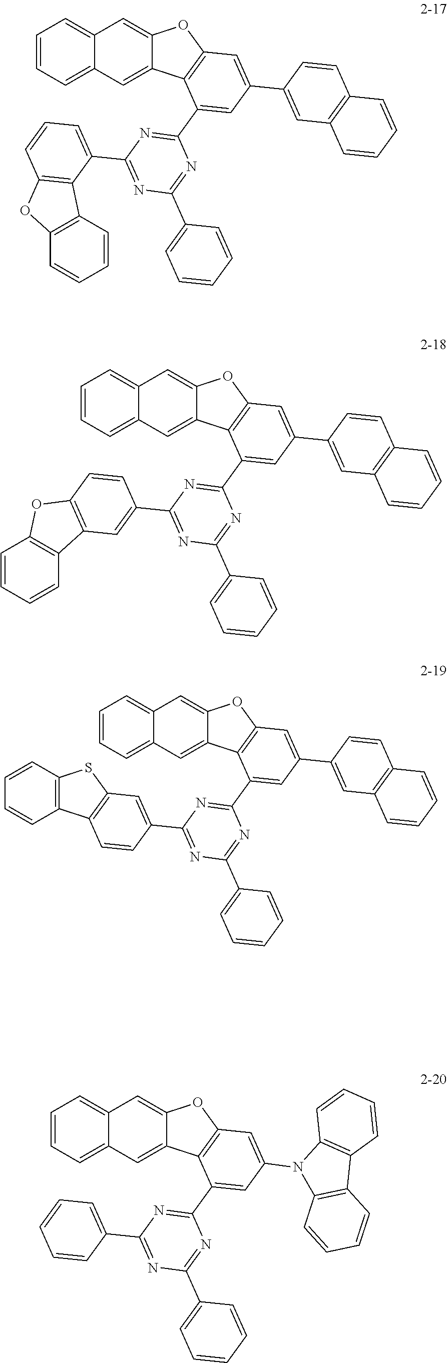 [SMILES: c1ccc(-c2nc(-c3cccc4oc5ccccc5c34)nc(-c3cc(-c4ccc5ccccc5c4)cc4oc5cc6ccccc6cc5c34)n2)cc1 ; c1ccc(-c2nc(-c3ccc4oc5ccccc5c4c3)nc(-c3cc(-c4ccc5ccccc5c4)cc4oc5cc6ccccc6cc5c34)n2)cc1 ; c1ccc(-c2nc(-c3ccc4c(c3)sc3ccccc34)nc(-c3cc(-c4ccc5ccccc5c4)cc4oc5cc6ccccc6cc5c34)n2)cc1 ; c1ccc(-c2nc(-c3ccccc3)nc(-c3cc(-n4c5ccccc5c5ccccc54)cc4oc5cc6ccccc6cc5c34)n2)cc1]

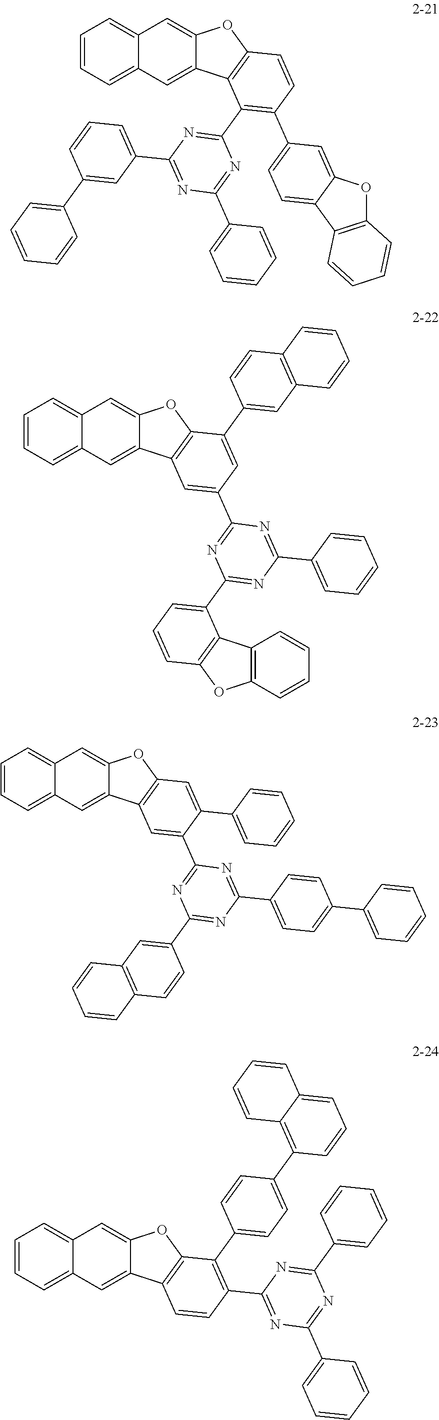 [SMILES: c1ccc(-c2cccc(-c3nc(-c4ccccc4)nc(-c4c(-c5ccc6c(c5)oc5ccccc56)ccc5oc6cc7ccccc7cc6c45)n3)c2)cc1 ; c1ccc(-c2nc(-c3cc(-c4ccc5ccccc5c4)c4oc5cc6ccccc6cc5c4c3)nc(-c3cccc4oc5ccccc5c34)n2)cc1 ; c1ccc(-c2ccc(-c3nc(-c4ccc5ccccc5c4)nc(-c4cc5c(cc4-c4ccccc4)oc4cc6ccccc6cc45)n3)cc2)cc1 ; c1ccc(-c2nc(-c3ccccc3)nc(-c3ccc4c(oc5cc6ccccc6cc54)c3-c3ccc(-c4cccc5ccccc45)cc3)n2)cc1]

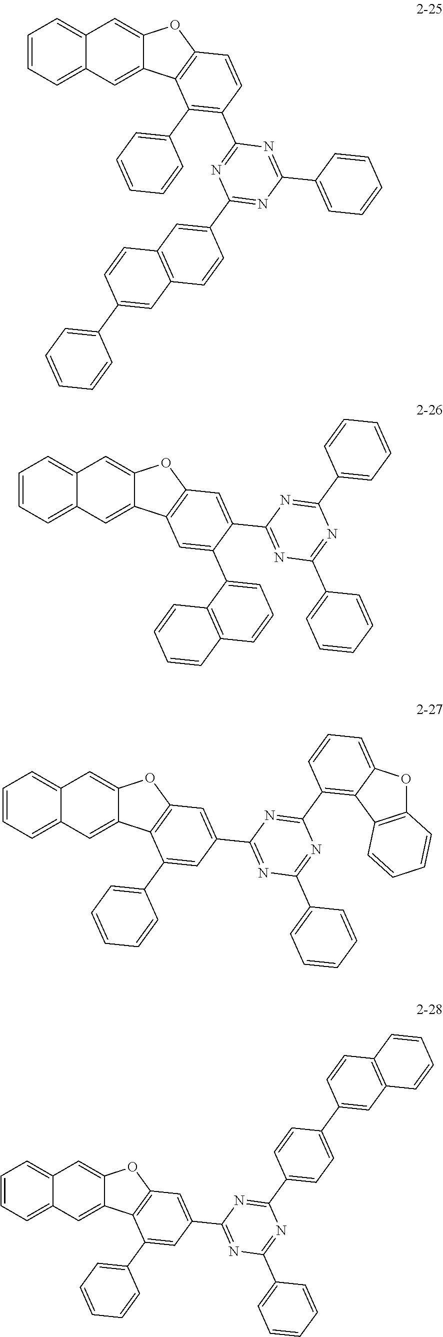 [SMILES: c1ccc(-c2ccc3cc(-c4nc(-c5ccccc5)nc(-c5ccc6oc7cc8ccccc8cc7c6c5-c5ccccc5)n4)ccc3c2)cc1 ; c1ccc(-c2nc(-c3ccccc3)nc(-c3cc4oc5cc6ccccc6cc5c4cc3-c3cccc4ccccc34)n2)cc1 ; c1ccc(-c2nc(-c3cc(-c4ccccc4)c4c(c3)oc3cc5ccccc5cc34)nc(-c3cccc4oc5ccccc5c34)n2)cc1 ; c1ccc(-c2nc(-c3ccc(-c4ccc5ccccc5c4)cc3)nc(-c3cc(-c4ccccc4)c4c(c3)oc3cc5ccccc5cc34)n2)cc1]

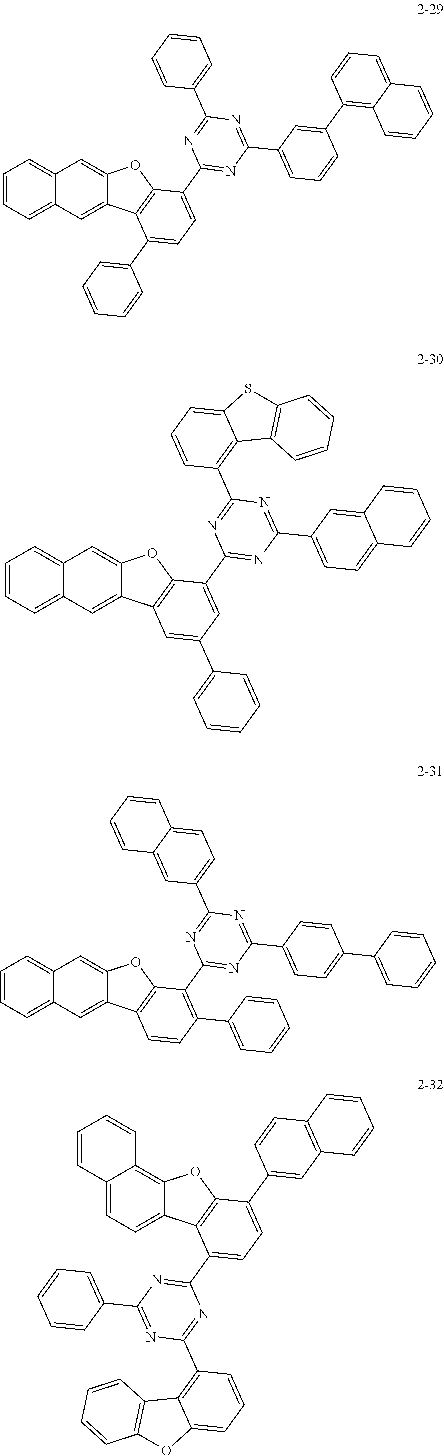 [SMILES: c1ccc(-c2nc(-c3cccc(-c4cccc5ccccc45)c3)nc(-c3ccc(-c4ccccc4)c4c3oc3cc5ccccc5cc34)n2)cc1 ; c1ccc(-c2cc(-c3nc(-c4ccc5ccccc5c4)nc(-c4cccc5sc6ccccc6c45)n3)c3oc4cc5ccccc5cc4c3c2)cc1 ; c1ccc(-c2ccc(-c3nc(-c4ccc5ccccc5c4)nc(-c4c(-c5ccccc5)ccc5c4oc4cc6ccccc6cc45)n3)cc2)cc1 ; c1ccc(-c2nc(-c3cccc4oc5ccccc5c34)nc(-c3ccc(-c4ccc5ccccc5c4)c4oc5c6ccccc6ccc5c34)n2)cc1]

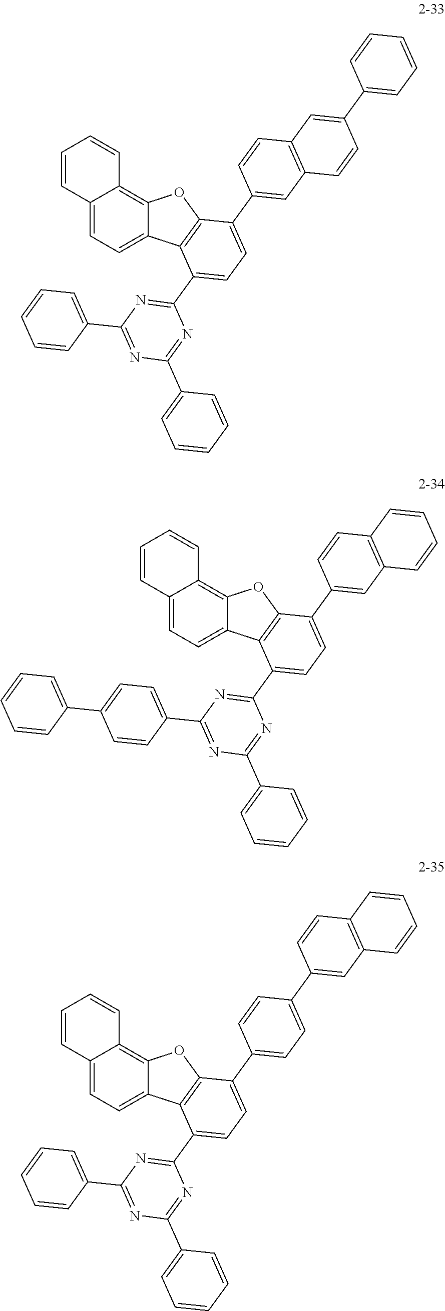 [SMILES: c1ccc(-c2ccc3cc(-c4ccc(-c5nc(-c6ccccc6)nc(-c6ccccc6)n5)c5c4oc4c6ccccc6ccc45)ccc3c2)cc1 ; c1ccc(-c2ccc(-c3nc(-c4ccccc4)nc(-c4ccc(-c5ccc6ccccc6c5)c5oc6c7ccccc7ccc6c45)n3)cc2)cc1 ; c1ccc(-c2nc(-c3ccccc3)nc(-c3ccc(-c4ccc(-c5ccc6ccccc6c5)cc4)c4oc5c6ccccc6ccc5c34)n2)cc1]

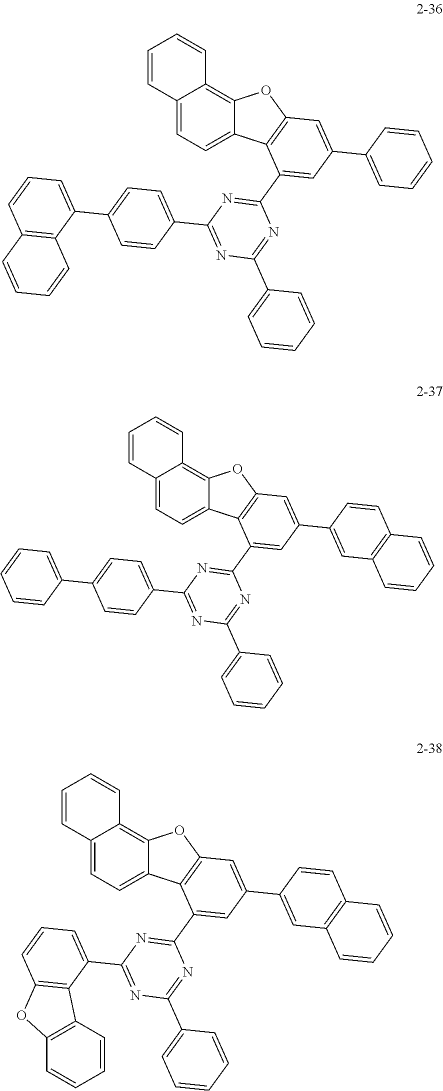 [SMILES: c1ccc(-c2cc(-c3nc(-c4ccccc4)nc(-c4ccc(-c5cccc6ccccc56)cc4)n3)c3c(c2)oc2c4ccccc4ccc23)cc1 ; c1ccc(-c2ccc(-c3nc(-c4ccccc4)nc(-c4cc(-c5ccc6ccccc6c5)cc5oc6c7ccccc7ccc6c45)n3)cc2)cc1 ; c1ccc(-c2nc(-c3cccc4oc5ccccc5c34)nc(-c3cc(-c4ccc5ccccc5c4)cc4oc5c6ccccc6ccc5c34)n2)cc1]

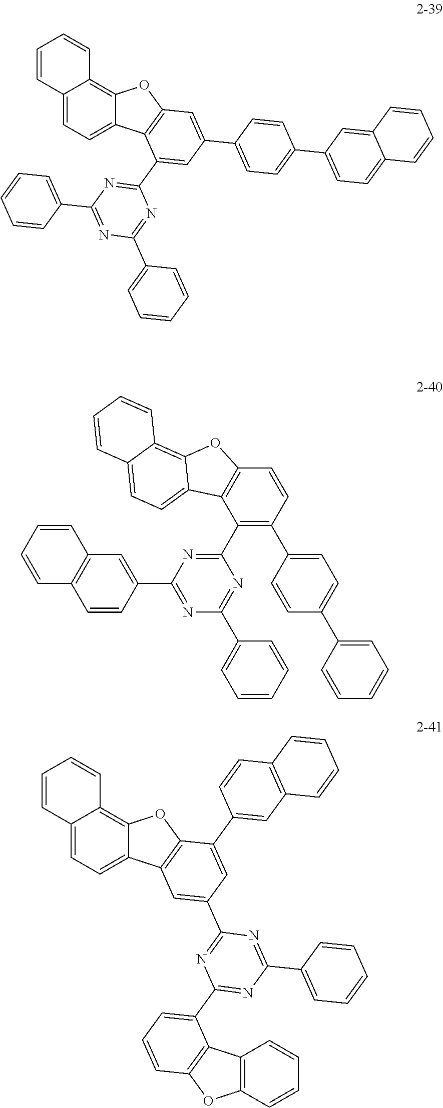

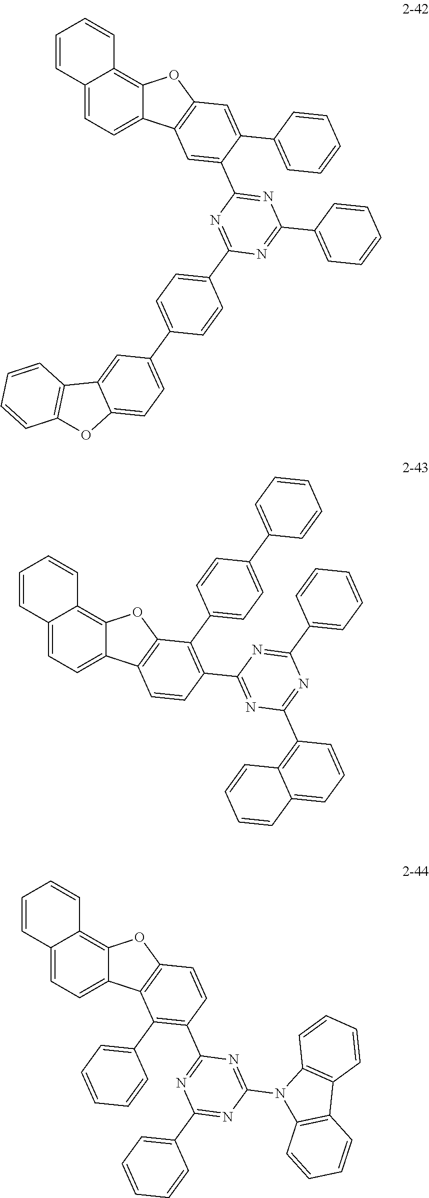 [SMILES: c1ccc(-c2nc(-c3ccc(-c4ccc5oc6ccccc6c5c4)cc3)nc(-c3cc4c(cc3-c3ccccc3)oc3c5ccccc5ccc43)n2)cc1 ; c1ccc(-c2ccc(-c3c(-c4nc(-c5ccccc5)nc(-c5cccc6ccccc56)n4)ccc4c3oc3c5ccccc5ccc43)cc2)cc1 ; c1ccc(-c2nc(-c3ccc4oc5c6ccccc6ccc5c4c3-c3ccccc3)nc(-n3c4ccccc4c4ccccc43)n2)cc1]

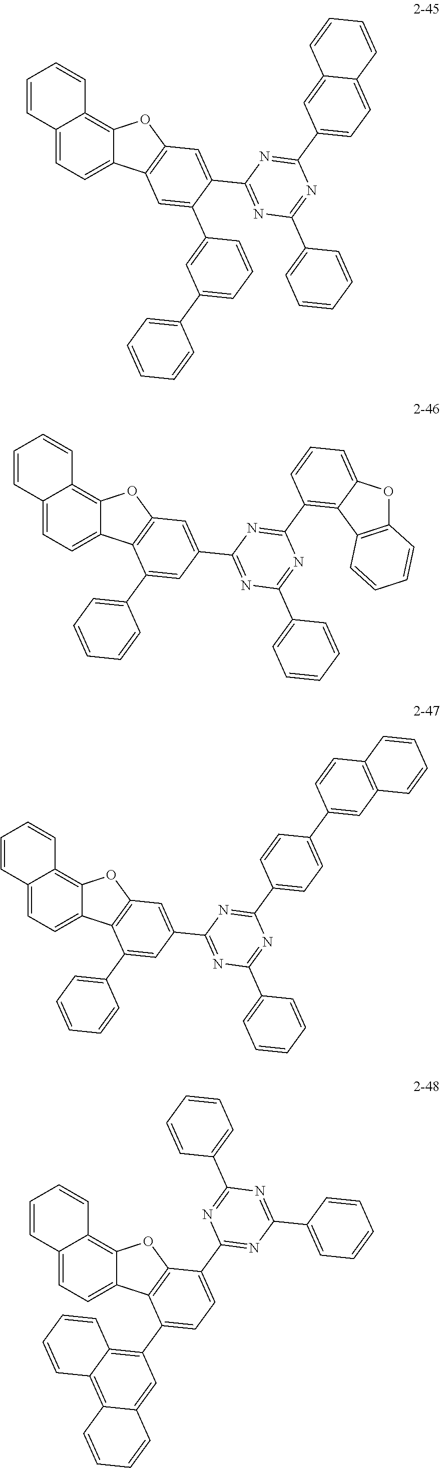 [SMILES: c1ccc(-c2cccc(-c3cc4c(cc3-c3nc(-c5ccccc5)nc(-c5ccc6ccccc6c5)n3)oc3c5ccccc5ccc43)c2)cc1 ; c1ccc(-c2nc(-c3cc(-c4ccccc4)c4c(c3)oc3c5ccccc5ccc34)nc(-c3cccc4oc5ccccc5c34)n2)cc1 ; c1ccc(-c2nc(-c3ccc(-c4ccc5ccccc5c4)cc3)nc(-c3cc(-c4ccccc4)c4c(c3)oc3c5ccccc5ccc34)n2)cc1 ; c1ccc(-c2nc(-c3ccccc3)nc(-c3ccc(-c4cc5ccccc5c5ccccc45)c4c3oc3c5ccccc5ccc34)n2)cc1]

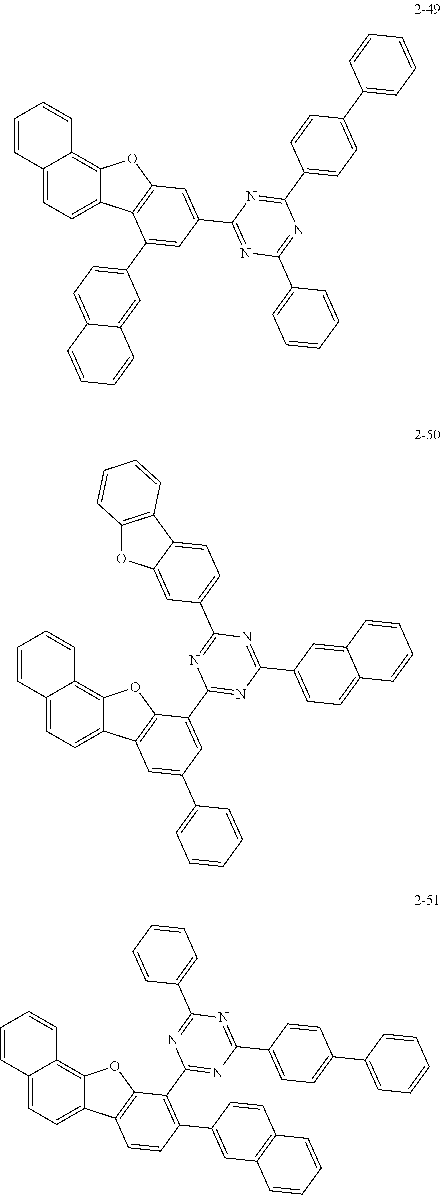 [SMILES: c1ccc(-c2ccc(-c3nc(-c4ccccc4)nc(-c4cc(-c5ccc6ccccc6c5)c5c(c4)oc4c6ccccc6ccc45)n3)cc2)cc1 ; c1ccc(-c2cc(-c3nc(-c4ccc5ccccc5c4)nc(-c4ccc5c(c4)oc4ccccc45)n3)c3oc4c5ccccc5ccc4c3c2)cc1 ; c1ccc(-c2ccc(-c3nc(-c4ccccc4)nc(-c4c(-c5ccc6ccccc6c5)ccc5c4oc4c6ccccc6ccc54)n3)cc2)cc1]

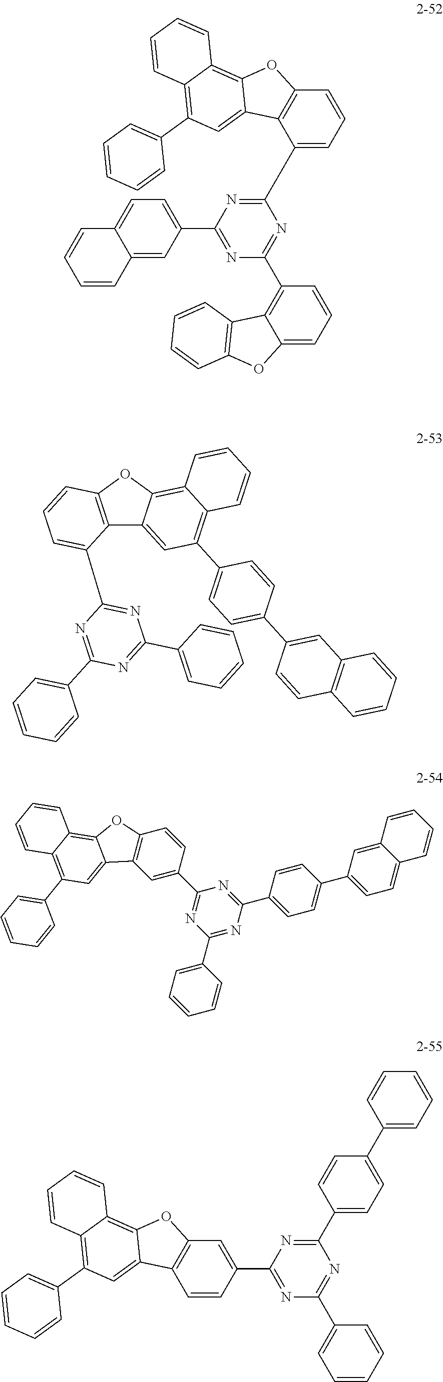 [SMILES: c1ccc(-c2cc3c(oc4cccc(-c5nc(-c6ccc7ccccc7c6)nc(-c6cccc7oc8ccccc8c67)n5)c43)c3ccccc23)cc1 ; c1ccc(-c2nc(-c3ccccc3)nc(-c3cccc4oc5c6ccccc6c(-c6ccc(-c7ccc8ccccc8c7)cc6)cc5c34)n2)cc1 ; c1ccc(-c2nc(-c3ccc(-c4ccc5ccccc5c4)cc3)nc(-c3ccc4oc5c6ccccc6c(-c6ccccc6)cc5c4c3)n2)cc1 ; c1ccc(-c2ccc(-c3nc(-c4ccccc4)nc(-c4ccc5c(c4)oc4c6ccccc6c(-c6ccccc6)cc54)n3)cc2)cc1]

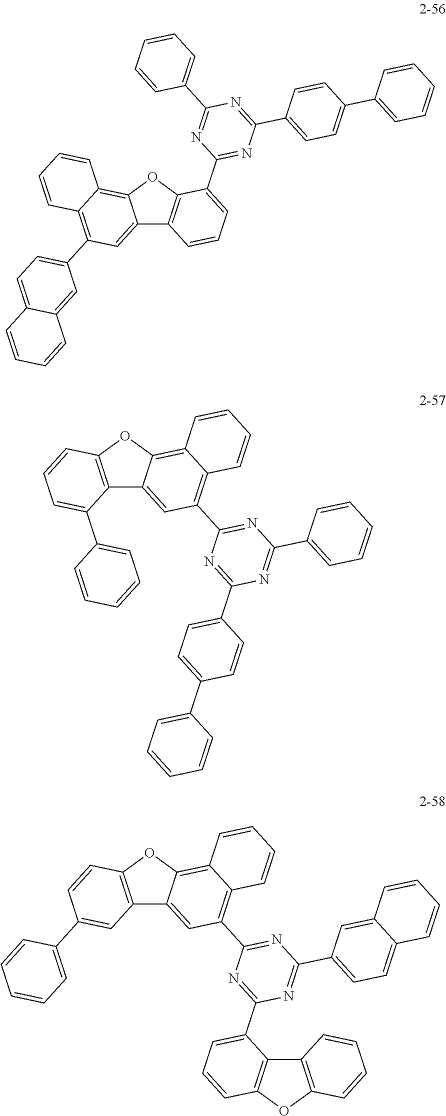 [SMILES: c1ccc(-c2ccc(-c3nc(-c4ccccc4)nc(-c4cccc5c4oc4c6ccccc6c(-c6ccc7ccccc7c6)cc54)n3)cc2)cc1 ; c1ccc(-c2ccc(-c3nc(-c4ccccc4)nc(-c4cc5c(oc6cccc(-c7ccccc7)c65)c5ccccc45)n3)cc2)cc1 ; c1ccc(-c2ccc3oc4c5ccccc5c(-c5nc(-c6ccc7ccccc7c6)nc(-c6cccc7oc8ccccc8c67)n5)cc4c3c2)cc1]

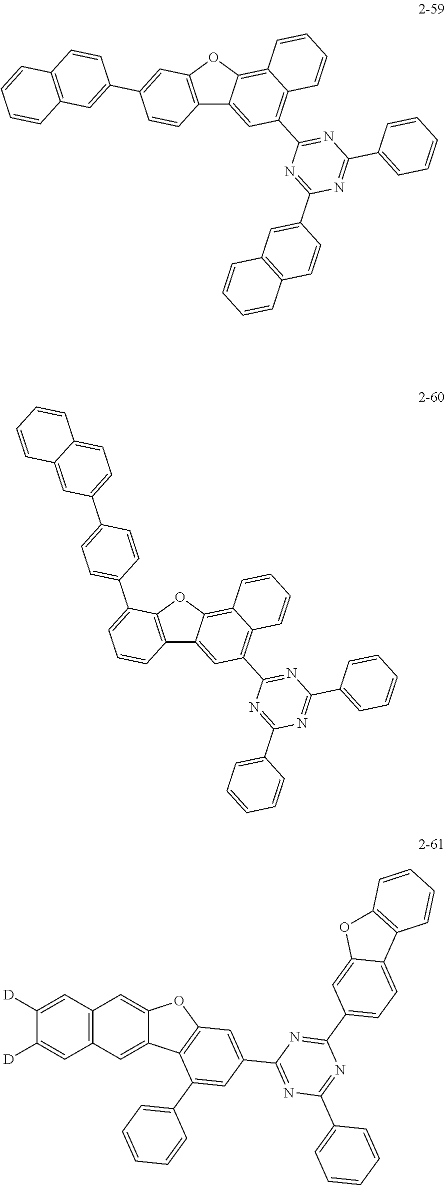 [SMILES: c1ccc(-c2nc(-c3ccc4ccccc4c3)nc(-c3cc4c5ccc(-c6ccc7ccccc7c6)cc5oc4c4ccccc34)n2)cc1 ; c1ccc(-c2nc(-c3ccccc3)nc(-c3cc4c5cccc(-c6ccc(-c7ccc8ccccc8c7)cc6)c5oc4c4ccccc34)n2)cc1 ; [2H]c1cc2cc3oc4cc(-c5nc(-c6ccccc6)nc(-c6ccc7c(c6)oc6ccccc67)n5)cc(-c5ccccc5)c4c3cc2cc1[2H]]

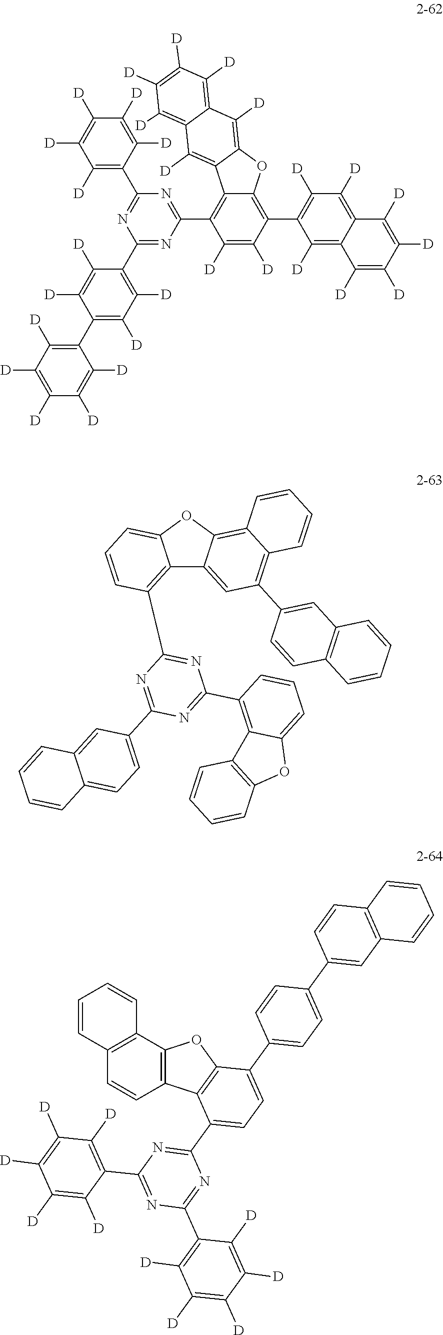 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-c4c([2H])c([2H])c5c([2H])c([2H])c([2H])c([2H])c5c4[2H])c4oc5c([2H])c6c([2H])c([2H])c([2H])c([2H])c6c([2H])c5c34)n2)c([2H])c1[2H] ; c1ccc2cc(-c3nc(-c4cccc5oc6ccccc6c45)nc(-c4cccc5oc6c7ccccc7c(-c7ccc8ccccc8c7)cc6c45)n3)ccc2c1 ; [2H]c1c([2H])c([2H])c(-c2nc(-c3c([2H])c([2H])c([2H])c([2H])c3[2H])nc(-c3ccc(-c4ccc(-c5ccc6ccccc6c5)cc4)c4oc5c6ccccc6ccc5c34)n2)c([2H])c1[2H]]

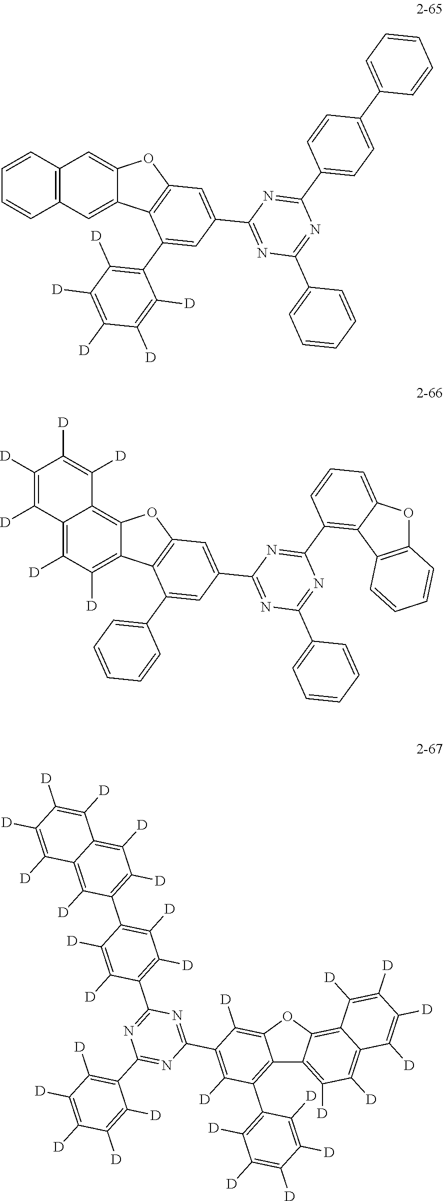 [SMILES: [2H]c1c([2H])c([2H])c(-c2cc(-c3nc(-c4ccccc4)nc(-c4ccc(-c5ccccc5)cc4)n3)cc3oc4cc5ccccc5cc4c23)c([2H])c1[2H] ; [2H]c1c([2H])c([2H])c2c(c1[2H])c([2H])c([2H])c1c2oc2cc(-c3nc(-c4ccccc4)nc(-c4cccc5oc6ccccc6c45)n3)cc(-c3ccccc3)c21 ; [2H]c1c([2H])c([2H])c(-c2nc(-c3c([2H])c([2H])c(-c4c([2H])c([2H])c5c([2H])c([2H])c([2H])c([2H])c5c4[2H])c([2H])c3[2H])nc(-c3c([2H])c(-c4c([2H])c([2H])c([2H])c([2H])c4[2H])c4c(oc5c6c([2H])c([2H])c([2H])c([2H])c6c([2H])c([2H])c54)c3[2H])n2)c([2H])c1[2H]]

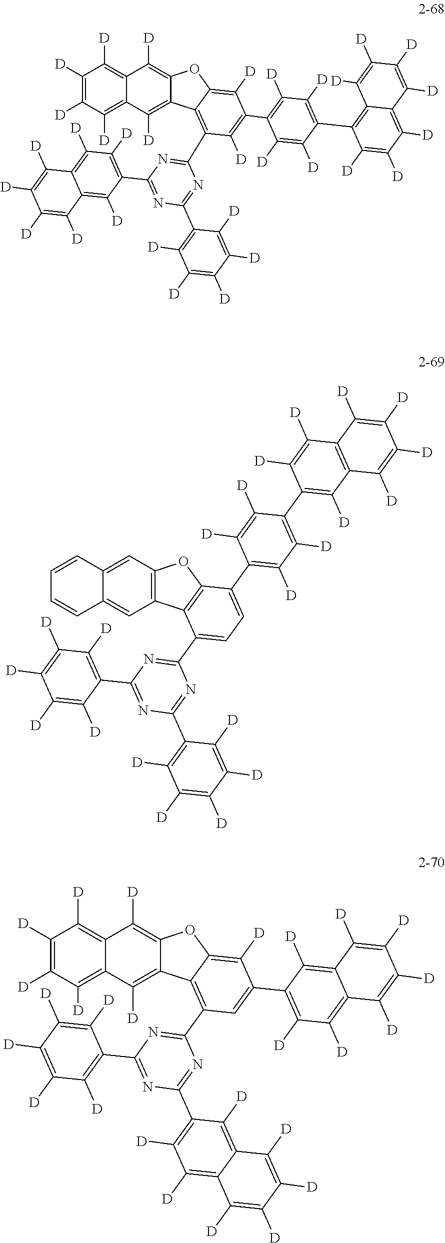

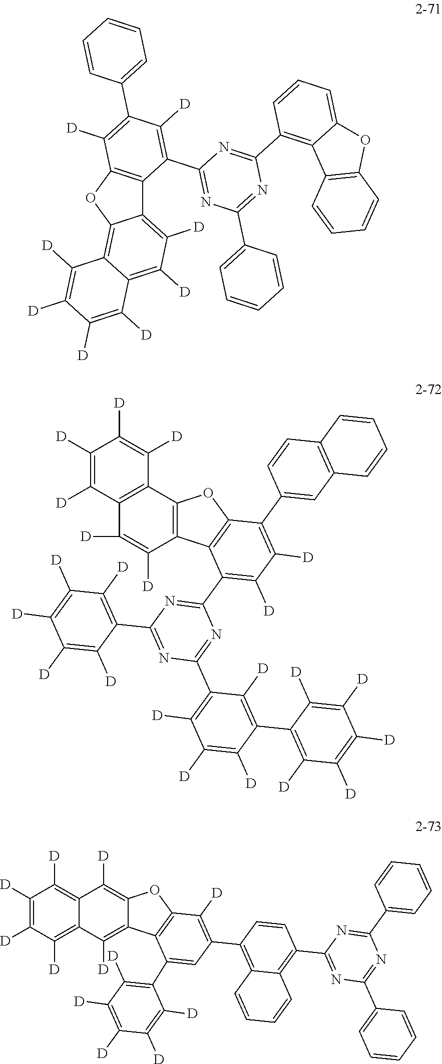 [SMILES: [2H]c1c([2H])c([2H])c2c(c1[2H])c([2H])c([2H])c1c2oc2c([2H])c(-c3ccccc3)c([2H])c(-c3nc(-c4ccccc4)nc(-c4cccc5oc6ccccc6c45)n3)c21 ; [2H]c1c([2H])c([2H])c(-c2nc(-c3c([2H])c([2H])c([2H])c(-c4c([2H])c([2H])c([2H])c([2H])c4[2H])c3[2H])nc(-c3c([2H])c([2H])c(-c4ccc5ccccc5c4)c4oc5c6c([2H])c([2H])c([2H])c([2H])c6c([2H])c([2H])c5c34)n2)c([2H])c1[2H] ; [2H]c1c([2H])c([2H])c(-c2cc(-c3ccc(-c4nc(-c5ccccc5)nc(-c5ccccc5)n4)c4ccccc34)c([2H])c3oc4c([2H])c5c([2H])c([2H])c([2H])c([2H])c5c([2H])c4c23)c([2H])c1[2H]]

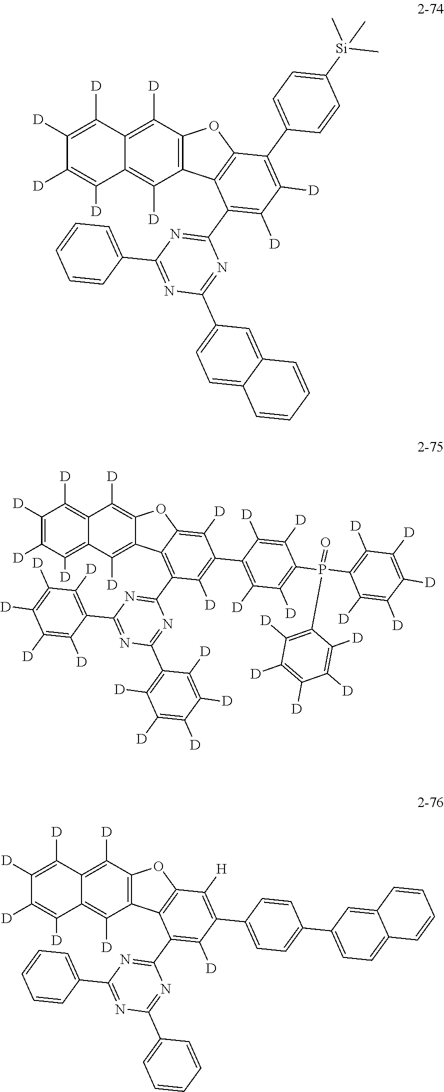

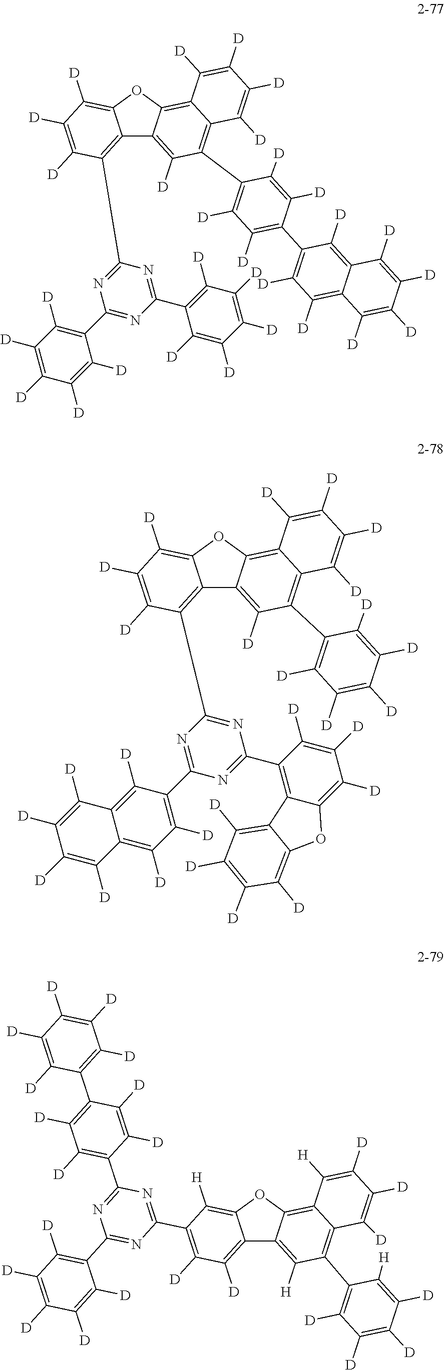

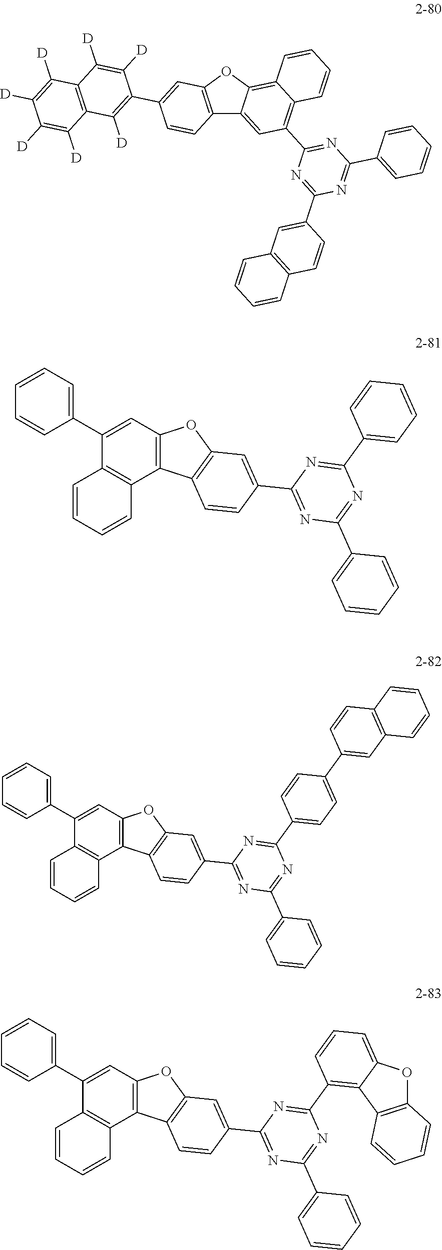 [SMILES: [2H]c1c([2H])c([2H])c2c([2H])c(-c3ccc4c(c3)oc3c5ccccc5c(-c5nc(-c6ccccc6)nc(-c6ccc7ccccc7c6)n5)cc43)c([2H])c([2H])c2c1[2H] ; c1ccc(-c2nc(-c3ccccc3)nc(-c3ccc4c(c3)oc3cc(-c5ccccc5)c5ccccc5c34)n2)cc1 ; c1ccc(-c2nc(-c3ccc(-c4ccc5ccccc5c4)cc3)nc(-c3ccc4c(c3)oc3cc(-c5ccccc5)c5ccccc5c34)n2)cc1 ; c1ccc(-c2nc(-c3ccc4c(c3)oc3cc(-c5ccccc5)c5ccccc5c34)nc(-c3cccc4oc5ccccc5c34)n2)cc1]

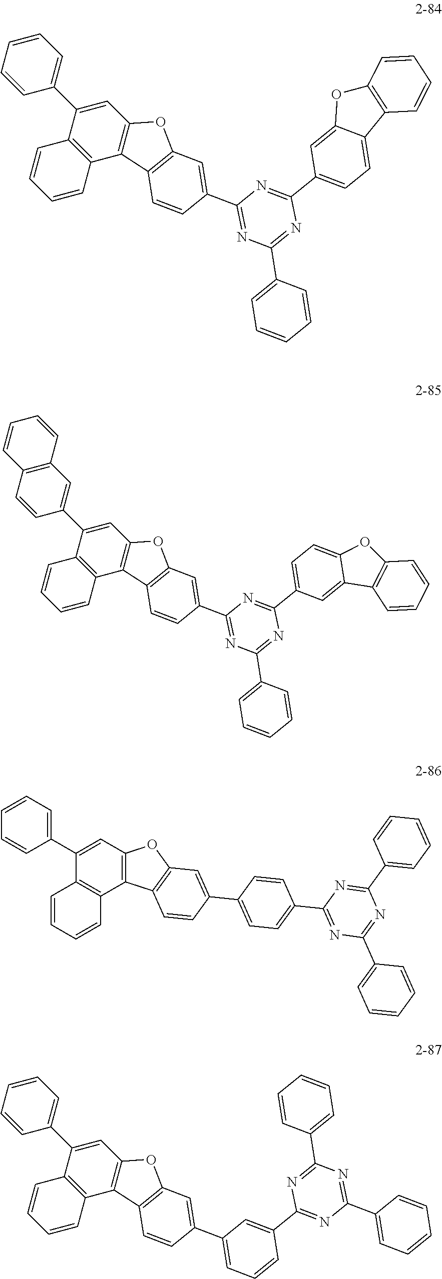 [SMILES: c1ccc(-c2nc(-c3ccc4c(c3)oc3ccccc34)nc(-c3ccc4c(c3)oc3cc(-c5ccccc5)c5ccccc5c34)n2)cc1 ; c1ccc(-c2nc(-c3ccc4c(c3)oc3cc(-c5ccc6ccccc6c5)c5ccccc5c34)nc(-c3ccc4oc5ccccc5c4c3)n2)cc1 ; c1ccc(-c2nc(-c3ccccc3)nc(-c3ccc(-c4ccc5c(c4)oc4cc(-c6ccccc6)c6ccccc6c45)cc3)n2)cc1 ; c1ccc(-c2nc(-c3ccccc3)nc(-c3cccc(-c4ccc5c(c4)oc4cc(-c6ccccc6)c6ccccc6c45)c3)n2)cc1]

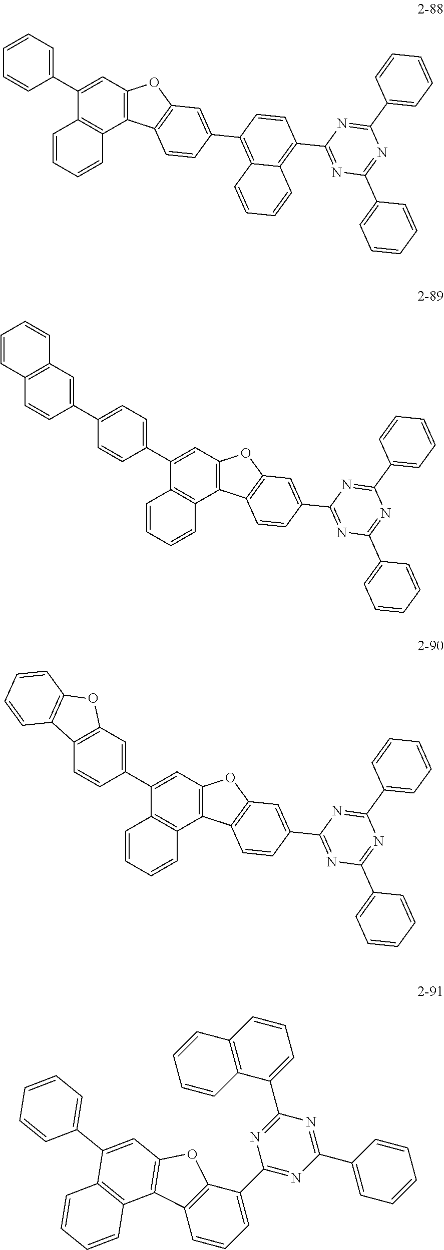 [SMILES: c1ccc(-c2nc(-c3ccccc3)nc(-c3ccc(-c4ccc5c(c4)oc4cc(-c6ccccc6)c6ccccc6c45)c4ccccc34)n2)cc1 ; c1ccc(-c2nc(-c3ccccc3)nc(-c3ccc4c(c3)oc3cc(-c5ccc(-c6ccc7ccccc7c6)cc5)c5ccccc5c34)n2)cc1 ; c1ccc(-c2nc(-c3ccccc3)nc(-c3ccc4c(c3)oc3cc(-c5ccc6c(c5)oc5ccccc56)c5ccccc5c34)n2)cc1 ; c1ccc(-c2nc(-c3cccc4ccccc34)nc(-c3cccc4c3oc3cc(-c5ccccc5)c5ccccc5c34)n2)cc1]

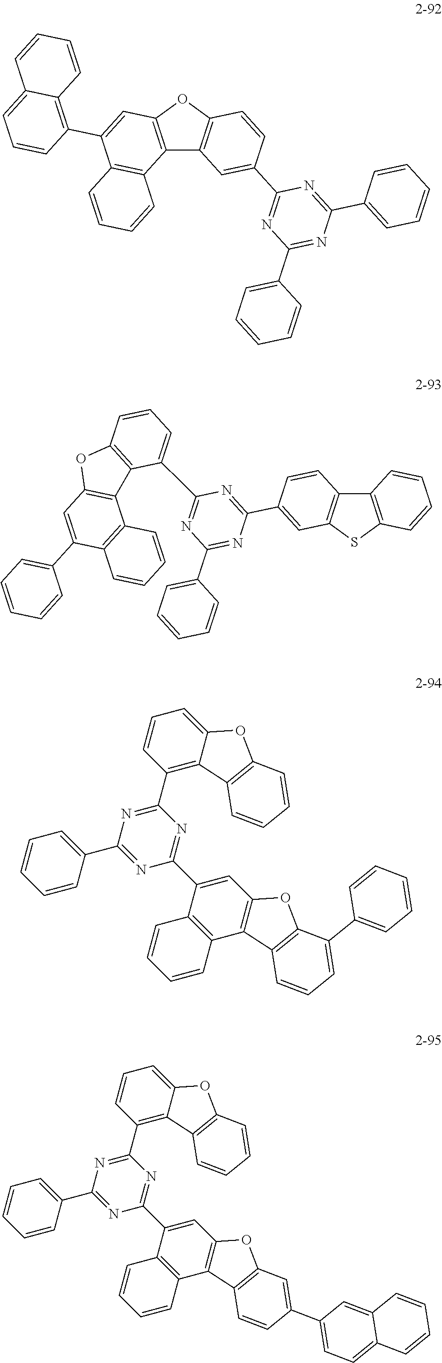 [SMILES: c1ccc(-c2nc(-c3ccccc3)nc(-c3ccc4oc5cc(-c6cccc7ccccc67)c6ccccc6c5c4c3)n2)cc1 ; c1ccc(-c2nc(-c3ccc4c(c3)sc3ccccc34)nc(-c3cccc4oc5cc(-c6ccccc6)c6ccccc6c5c34)n2)cc1 ; c1ccc(-c2nc(-c3cc4oc5c(-c6ccccc6)cccc5c4c4ccccc34)nc(-c3cccc4oc5ccccc5c34)n2)cc1 ; c1ccc(-c2nc(-c3cc4oc5cc(-c6ccc7ccccc7c6)ccc5c4c4ccccc34)nc(-c3cccc4oc5ccccc5c34)n2)cc1]

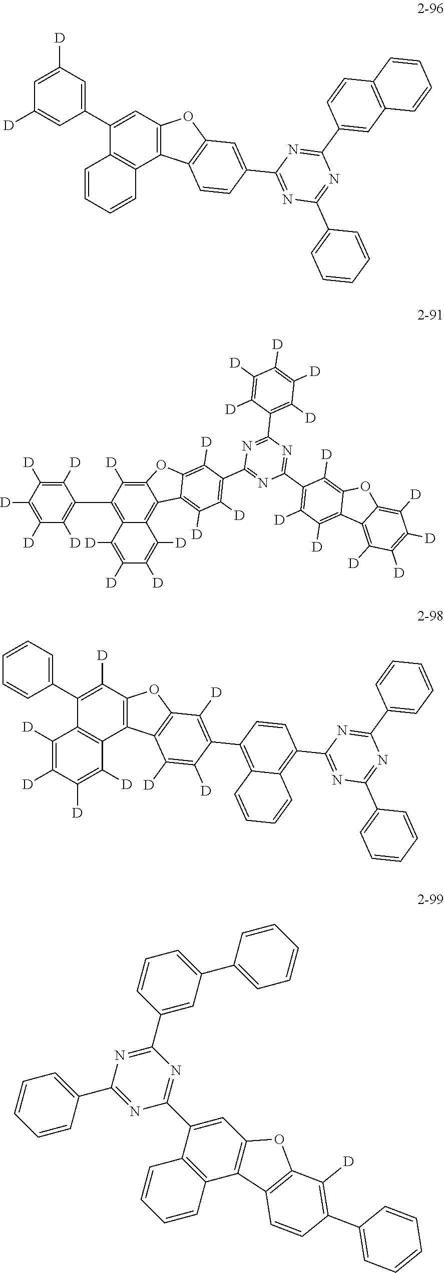

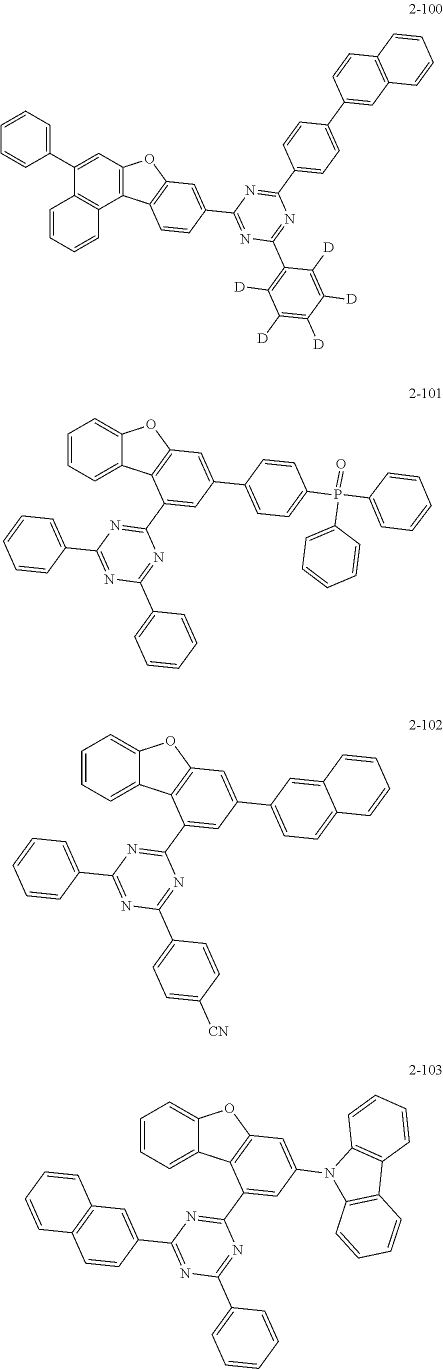 [SMILES: [2H]c1c([2H])c([2H])c(-c2nc(-c3ccc(-c4ccc5ccccc5c4)cc3)nc(-c3ccc4c(c3)oc3cc(-c5ccccc5)c5ccccc5c34)n2)c([2H])c1[2H] ; O=P(c1ccccc1)(c1ccccc1)c1ccc(-c2cc(-c3nc(-c4ccccc4)nc(-c4ccccc4)n3)c3c(c2)oc2ccccc23)cc1 ; N#Cc1ccc(-c2nc(-c3ccccc3)nc(-c3cc(-c4ccc5ccccc5c4)cc4oc5ccccc5c34)n2)cc1 ; c1ccc(-c2nc(-c3ccc4ccccc4c3)nc(-c3cc(-n4c5ccccc5c5ccccc54)cc4oc5ccccc5c34)n2)cc1]

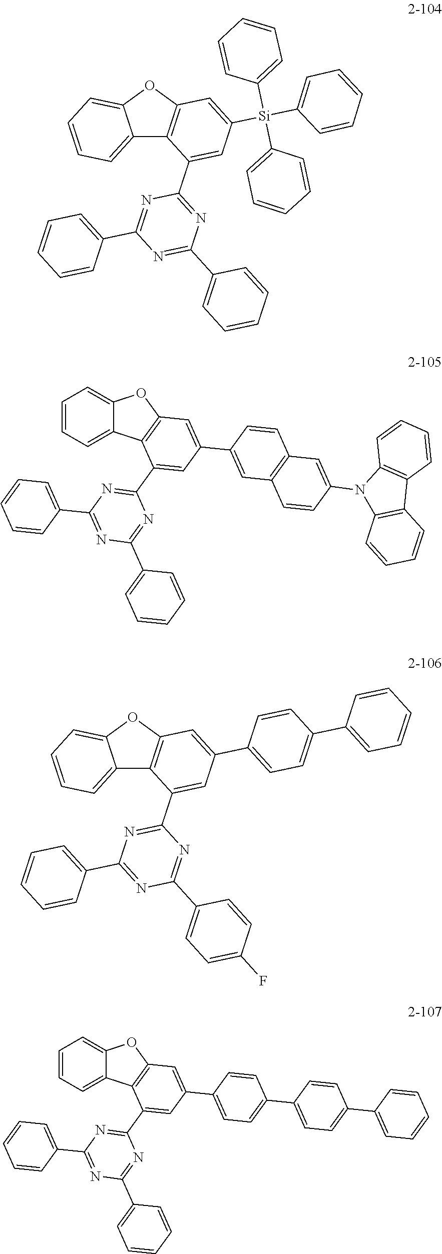 [SMILES: c1ccc(-c2nc(-c3ccccc3)nc(-c3cc([Si](c4ccccc4)(c4ccccc4)c4ccccc4)cc4oc5ccccc5c34)n2)cc1 ; c1ccc(-c2nc(-c3ccccc3)nc(-c3cc(-c4ccc5cc(-n6c7ccccc7c7ccccc76)ccc5c4)cc4oc5ccccc5c34)n2)cc1 ; Fc1ccc(-c2nc(-c3ccccc3)nc(-c3cc(-c4ccc(-c5ccccc5)cc4)cc4oc5ccccc5c34)n2)cc1 ; c1ccc(-c2ccc(-c3ccc(-c4cc(-c5nc(-c6ccccc6)nc(-c6ccccc6)n5)c5c(c4)oc4ccccc45)cc3)cc2)cc1]

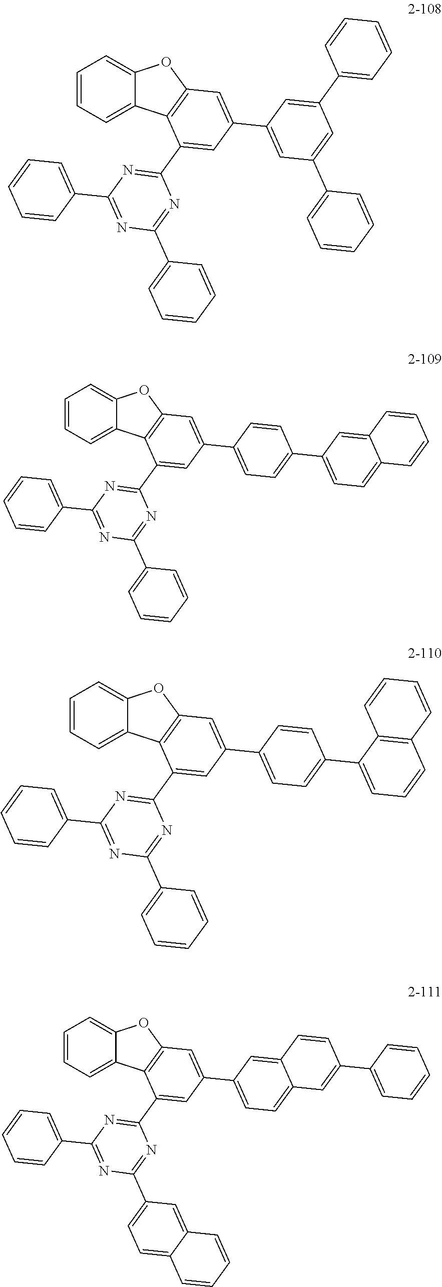 [SMILES: c1ccc(-c2cc(-c3ccccc3)cc(-c3cc(-c4nc(-c5ccccc5)nc(-c5ccccc5)n4)c4c(c3)oc3ccccc34)c2)cc1 ; c1ccc(-c2nc(-c3ccccc3)nc(-c3cc(-c4ccc(-c5ccc6ccccc6c5)cc4)cc4oc5ccccc5c34)n2)cc1 ; c1ccc(-c2nc(-c3ccccc3)nc(-c3cc(-c4ccc(-c5cccc6ccccc56)cc4)cc4oc5ccccc5c34)n2)cc1 ; c1ccc(-c2ccc3cc(-c4cc(-c5nc(-c6ccccc6)nc(-c6ccc7ccccc7c6)n5)c5c(c4)oc4ccccc45)ccc3c2)cc1]

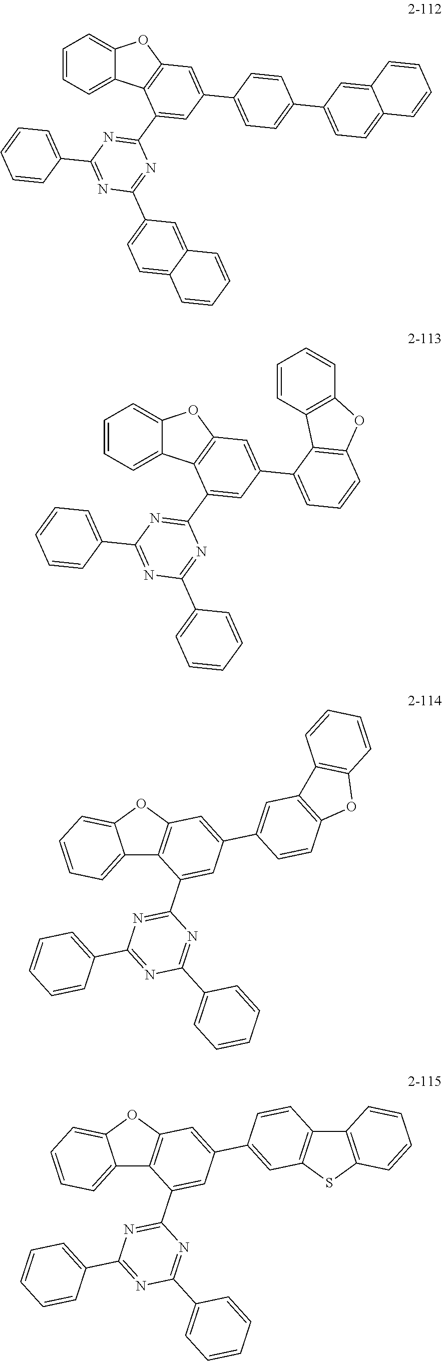 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2nc(-c3ccccc3)nc(-c3cc(-c4cccc5oc6ccccc6c45)cc4oc5ccccc5c34)n2)cc1 ; c1ccc(-c2nc(-c3ccccc3)nc(-c3cc(-c4ccc5oc6ccccc6c5c4)cc4oc5ccccc5c34)n2)cc1 ; c1ccc(-c2nc(-c3ccccc3)nc(-c3cc(-c4ccc5c(c4)sc4ccccc45)cc4oc5ccccc5c34)n2)cc1]

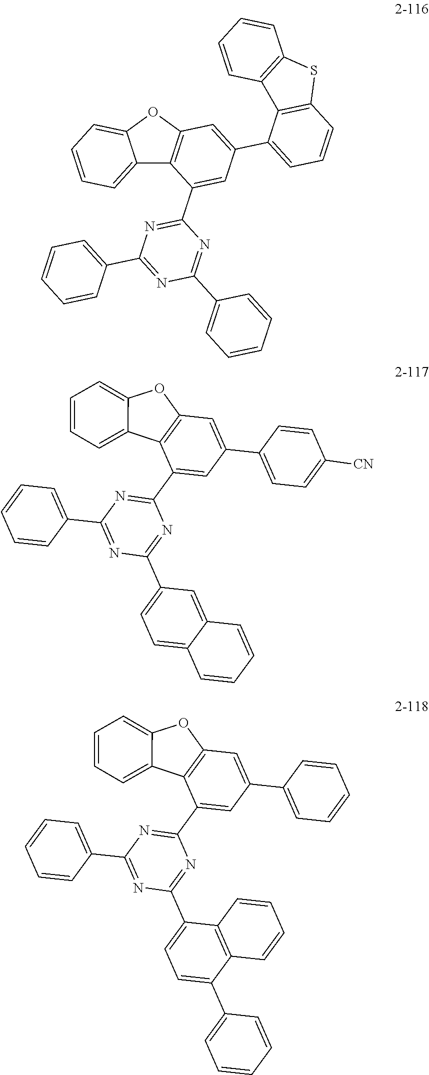 [SMILES: c1ccc(-c2nc(-c3ccccc3)nc(-c3cc(-c4cccc5sc6ccccc6c45)cc4oc5ccccc5c34)n2)cc1 ; N#Cc1ccc(-c2cc(-c3nc(-c4ccccc4)nc(-c4ccc5ccccc5c4)n3)c3c(c2)oc2ccccc23)cc1 ; c1ccc(-c2cc(-c3nc(-c4ccccc4)nc(-c4ccc(-c5ccccc5)c5ccccc45)n3)c3c(c2)oc2ccccc23)cc1]

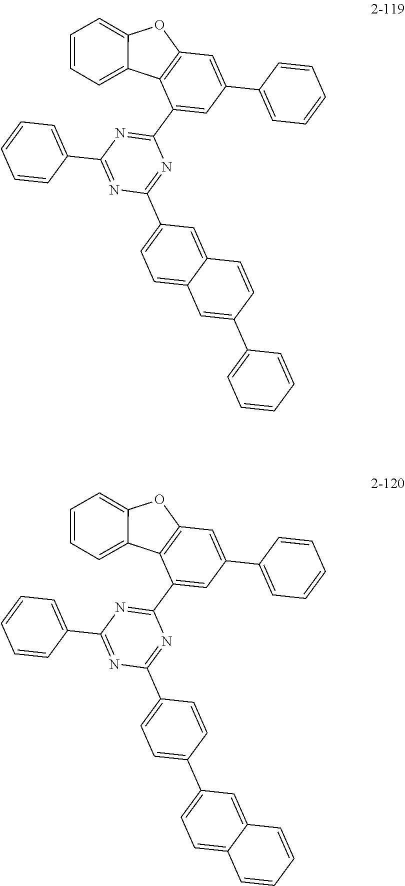 [SMILES: c1ccc(-c2ccc3cc(-c4nc(-c5ccccc5)nc(-c5cc(-c6ccccc6)cc6oc7ccccc7c56)n4)ccc3c2)cc1 ; c1ccc(-c2cc(-c3nc(-c4ccccc4)nc(-c4ccc(-c5ccc6ccccc6c5)cc4)n3)c3c(c2)oc2ccccc23)cc1]

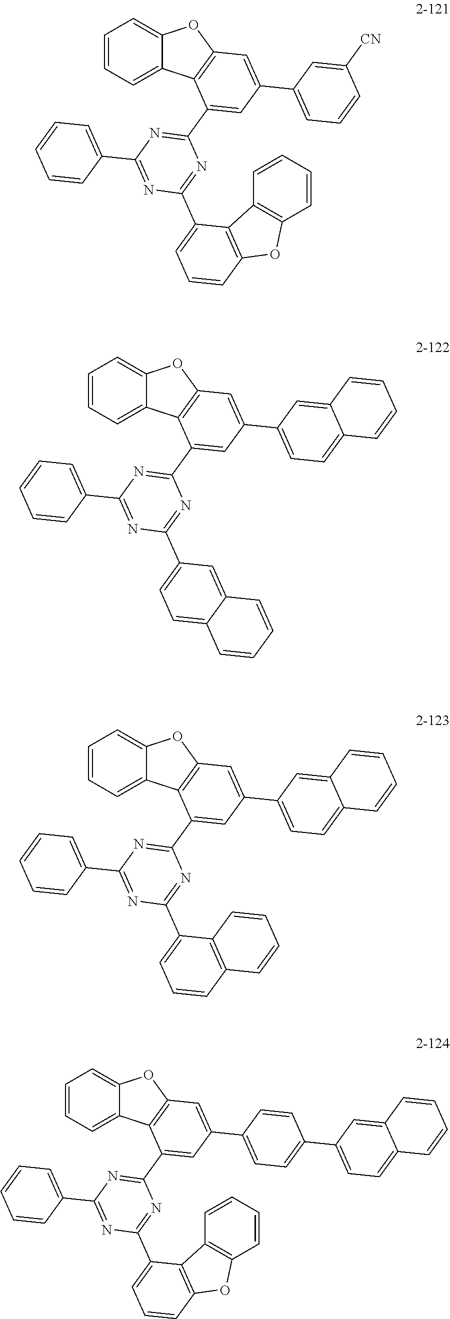 [SMILES: N#Cc1cccc(-c2cc(-c3nc(-c4ccccc4)nc(-c4cccc5oc6ccccc6c45)n3)c3c(c2)oc2ccccc23)c1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cccc4ccccc34)nc(-c3cc(-c4ccc5ccccc5c4)cc4oc5ccccc5c34)n2)cc1 ; c1ccc(-c2nc(-c3cccc4oc5ccccc5c34)nc(-c3cc(-c4ccc(-c5ccc6ccccc6c5)cc4)cc4oc5ccccc5c34)n2)cc1]

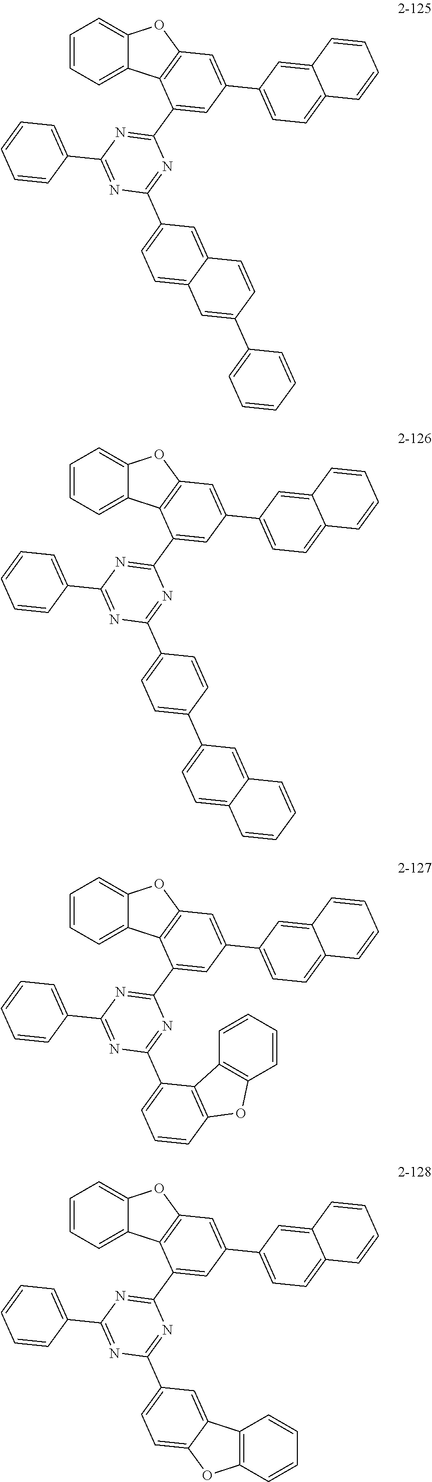 [SMILES: c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3ccc4oc5ccccc5c4c3)nc(-c3cc(-c4ccc5ccccc5c4)cc4oc5ccccc5c34)n2)cc1]

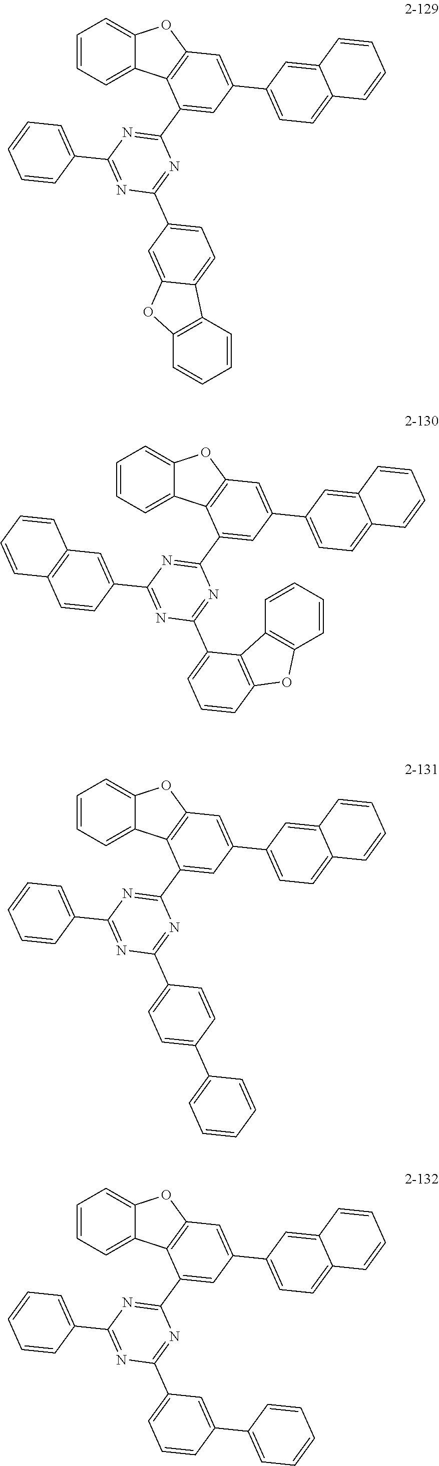 [SMILES: c1ccc(-c2nc(-c3ccc4c(c3)oc3ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5cccc6oc7ccccc7c56)n4)c4c(c3)oc3ccccc34)ccc2c1 ; c1ccc(-c2ccc(-c3nc(-c4ccccc4)nc(-c4cc(-c5ccc6ccccc6c5)cc5oc6ccccc6c45)n3)cc2)cc1 ; c1ccc(-c2cccc(-c3nc(-c4ccccc4)nc(-c4cc(-c5ccc6ccccc6c5)cc5oc6ccccc6c45)n3)c2)cc1]

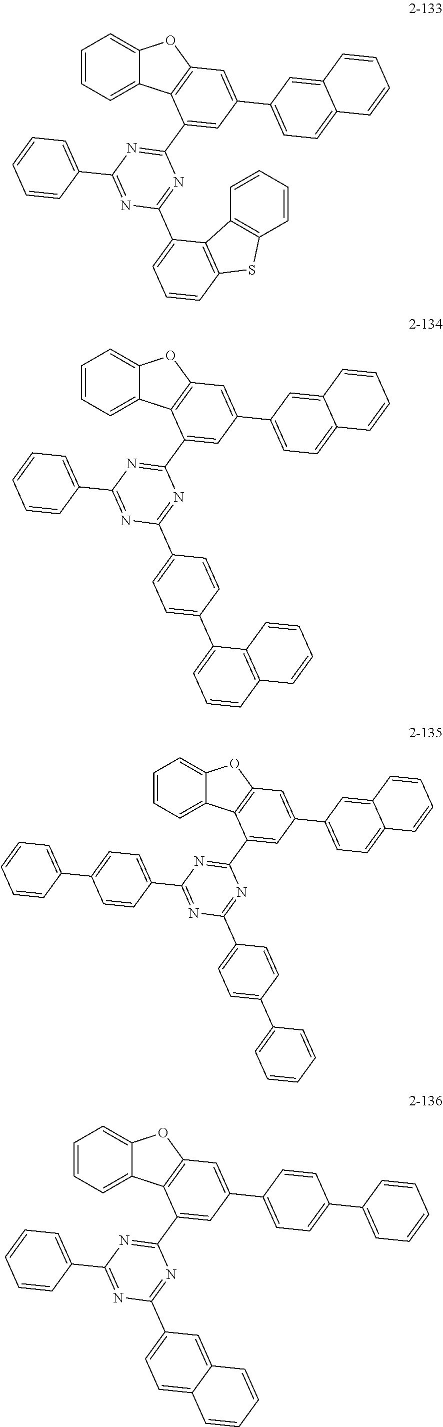 [SMILES: c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3cc(-c4nc(-c5ccccc5)nc(-c5ccc6ccccc6c5)n4)c4c(c3)oc3ccccc34)cc2)cc1]

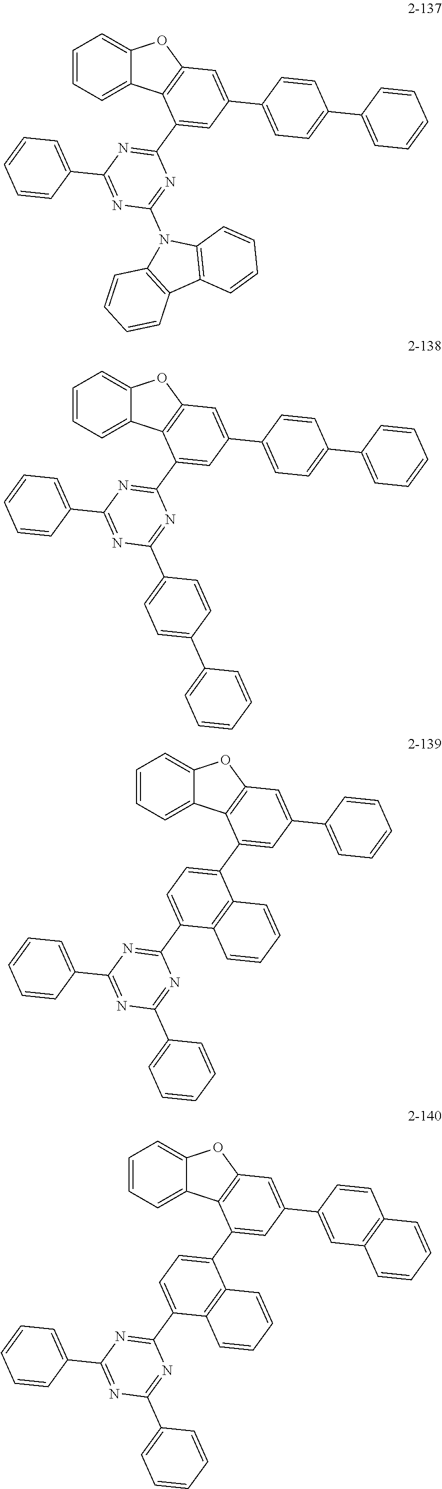

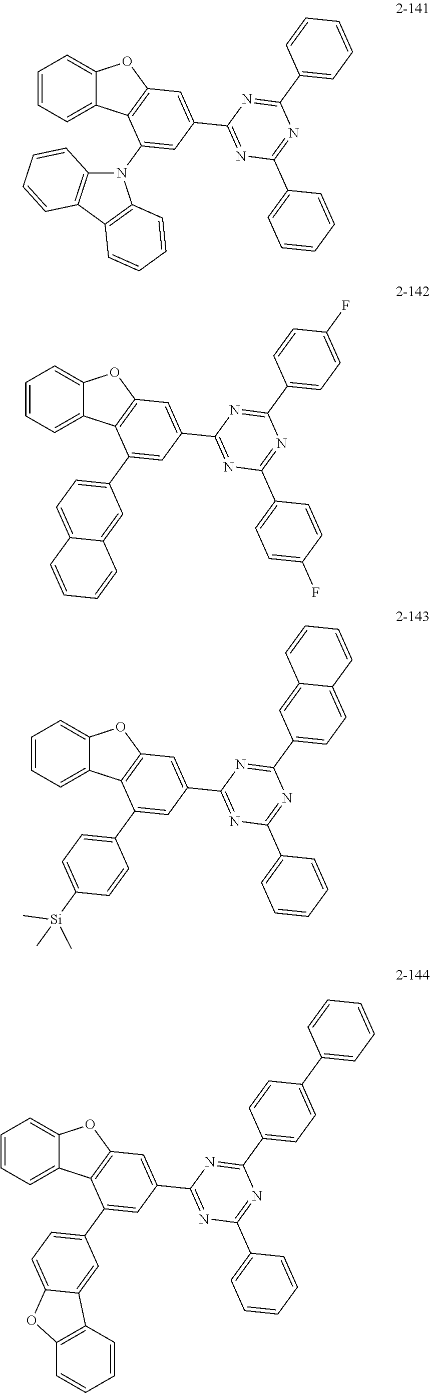

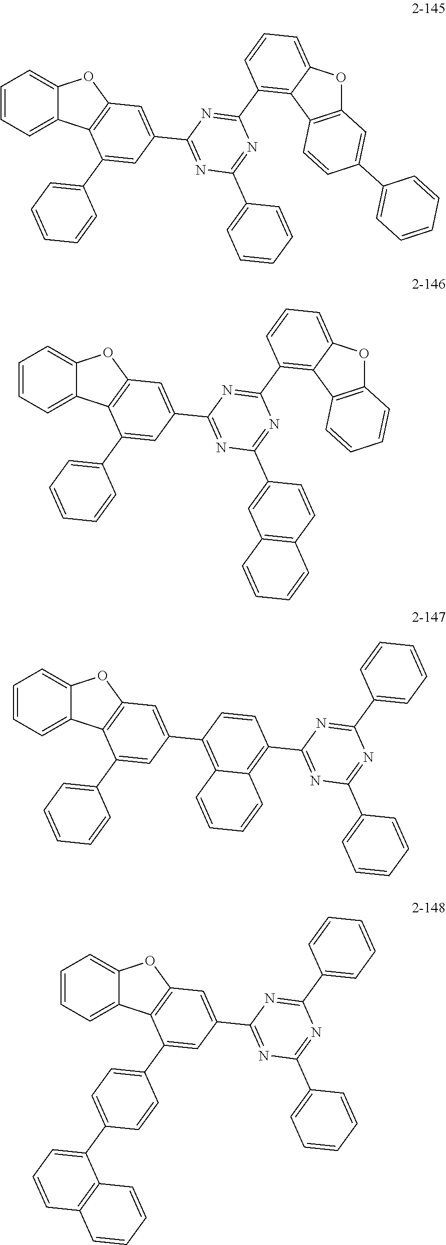 [SMILES: c1ccc(-c2ccc3c(c2)oc2cccc(-c4nc(-c5ccccc5)nc(-c5cc(-c6ccccc6)c6c(c5)oc5ccccc56)n4)c23)cc1 ; c1ccc(-c2cc(-c3nc(-c4ccc5ccccc5c4)nc(-c4cccc5oc6ccccc6c45)n3)cc3oc4ccccc4c23)cc1 ; c1ccc(-c2nc(-c3ccccc3)nc(-c3ccc(-c4cc(-c5ccccc5)c5c(c4)oc4ccccc45)c4ccccc34)n2)cc1 ; c1ccc(-c2nc(-c3ccccc3)nc(-c3cc(-c4ccc(-c5cccc6ccccc56)cc4)c4c(c3)oc3ccccc34)n2)cc1]

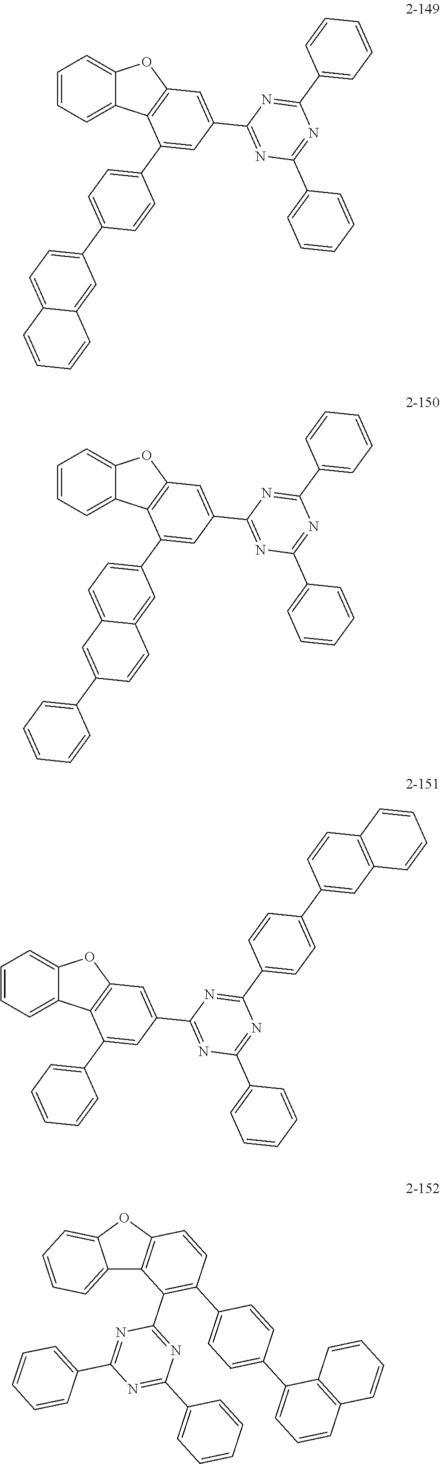 [SMILES: c1ccc(-c2nc(-c3ccccc3)nc(-c3cc(-c4ccc(-c5ccc6ccccc6c5)cc4)c4c(c3)oc3ccccc34)n2)cc1 ; c1ccc(-c2ccc3cc(-c4cc(-c5nc(-c6ccccc6)nc(-c6ccccc6)n5)cc5oc6ccccc6c45)ccc3c2)cc1 ; c1ccc(-c2nc(-c3ccc(-c4ccc5ccccc5c4)cc3)nc(-c3cc(-c4ccccc4)c4c(c3)oc3ccccc34)n2)cc1 ; c1ccc(-c2nc(-c3ccccc3)nc(-c3c(-c4ccc(-c5cccc6ccccc56)cc4)ccc4oc5ccccc5c34)n2)cc1]

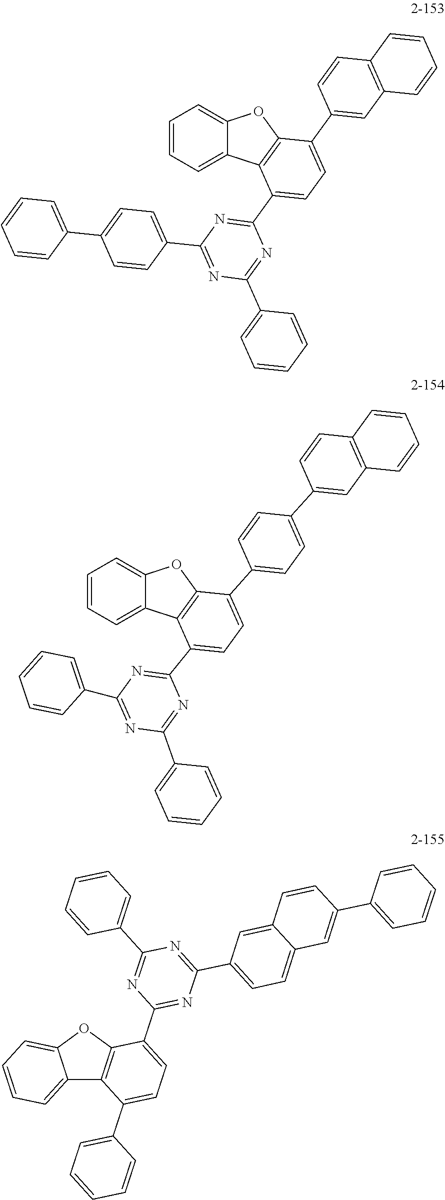 [SMILES: c1ccc(-c2ccc(-c3nc(-c4ccccc4)nc(-c4ccc(-c5ccc6ccccc6c5)c5oc6ccccc6c45)n3)cc2)cc1 ; c1ccc(-c2nc(-c3ccccc3)nc(-c3ccc(-c4ccc(-c5ccc6ccccc6c5)cc4)c4oc5ccccc5c34)n2)cc1 ; c1ccc(-c2ccc3cc(-c4nc(-c5ccccc5)nc(-c5ccc(-c6ccccc6)c6c5oc5ccccc56)n4)ccc3c2)cc1]

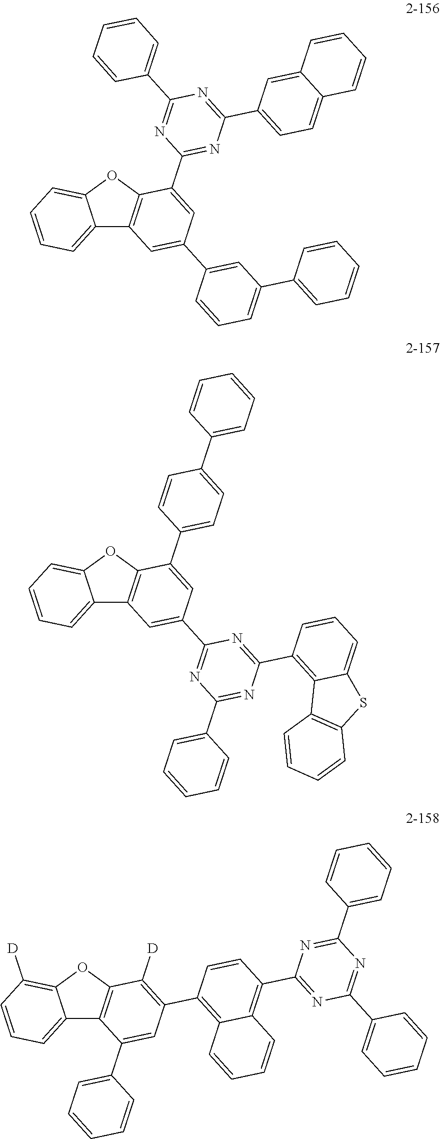 [SMILES: c1ccc(-c2cccc(-c3cc(-c4nc(-c5ccccc5)nc(-c5ccc6ccccc6c5)n4)c4oc5ccccc5c4c3)c2)cc1 ; c1ccc(-c2ccc(-c3cc(-c4nc(-c5ccccc5)nc(-c5cccc6sc7ccccc7c56)n4)cc4c3oc3ccccc34)cc2)cc1 ; [2H]c1cccc2c1oc1c([2H])c(-c3ccc(-c4nc(-c5ccccc5)nc(-c5ccccc5)n4)c4ccccc34)cc(-c3ccccc3)c12]

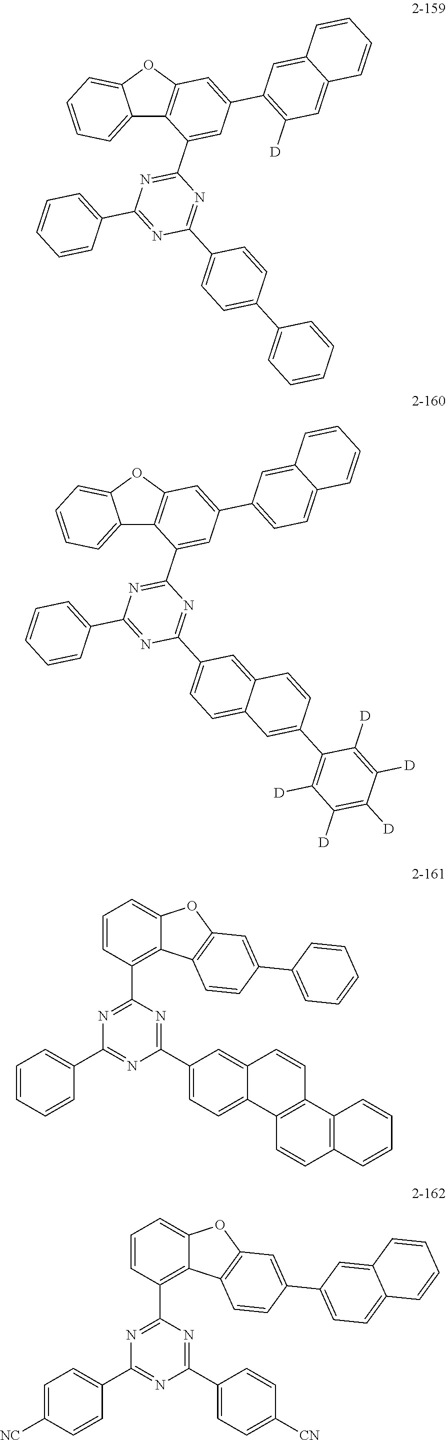 [SMILES: [2H]c1cc2ccccc2cc1-c1cc(-c2nc(-c3ccccc3)nc(-c3ccc(-c4ccccc4)cc3)n2)c2c(c1)oc1ccccc12 ; [2H]c1c([2H])c([2H])c(-c2ccc3cc(-c4nc(-c5ccccc5)nc(-c5cc(-c6ccc7ccccc7c6)cc6oc7ccccc7c56)n4)ccc3c2)c([2H])c1[2H] ; c1ccc(-c2ccc3c(c2)oc2cccc(-c4nc(-c5ccccc5)nc(-c5ccc6c(ccc7c8ccccc8ccc67)c5)n4)c23)cc1 ; N#Cc1ccc(-c2nc(-c3ccc(C#N)cc3)nc(-c3cccc4oc5cc(-c6ccc7ccccc7c6)ccc5c34)n2)cc1]

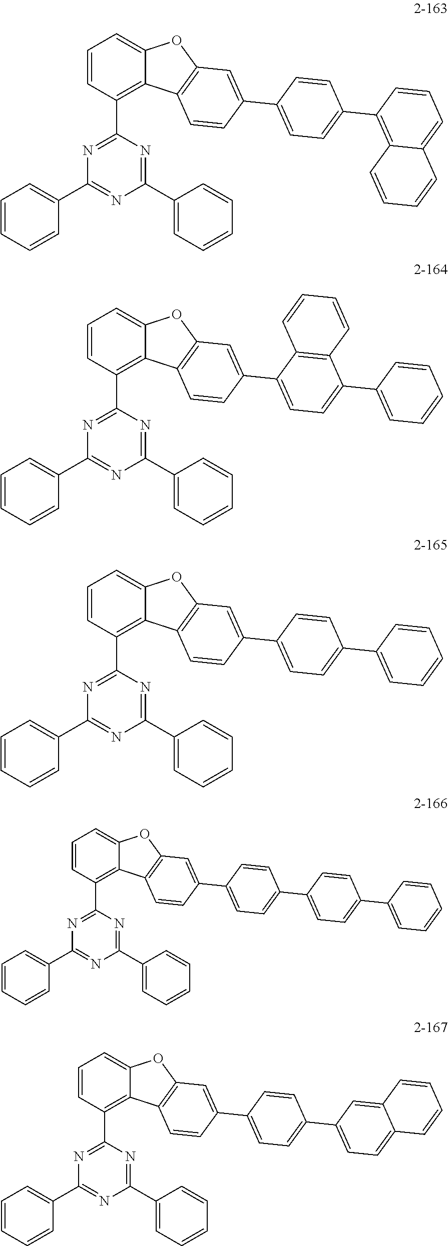 [SMILES: c1ccc(-c2nc(-c3ccccc3)nc(-c3cccc4oc5cc(-c6ccc(-c7cccc8ccccc78)cc6)ccc5c34)n2)cc1 ; c1ccc(-c2nc(-c3ccccc3)nc(-c3cccc4oc5cc(-c6ccc(-c7ccccc7)c7ccccc67)ccc5c34)n2)cc1 ; c1ccc(-c2ccc(-c3ccc4c(c3)oc3cccc(-c5nc(-c6ccccc6)nc(-c6ccccc6)n5)c34)cc2)cc1 ; c1ccc(-c2ccc(-c3ccc(-c4ccc5c(c4)oc4cccc(-c6nc(-c7ccccc7)nc(-c7ccccc7)n6)c45)cc3)cc2)cc1 ; c1ccc(-c2nc(-c3ccccc3)nc(-c3cccc4oc5cc(-c6ccc(-c7ccc8ccccc8c7)cc6)ccc5c34)n2)cc1]

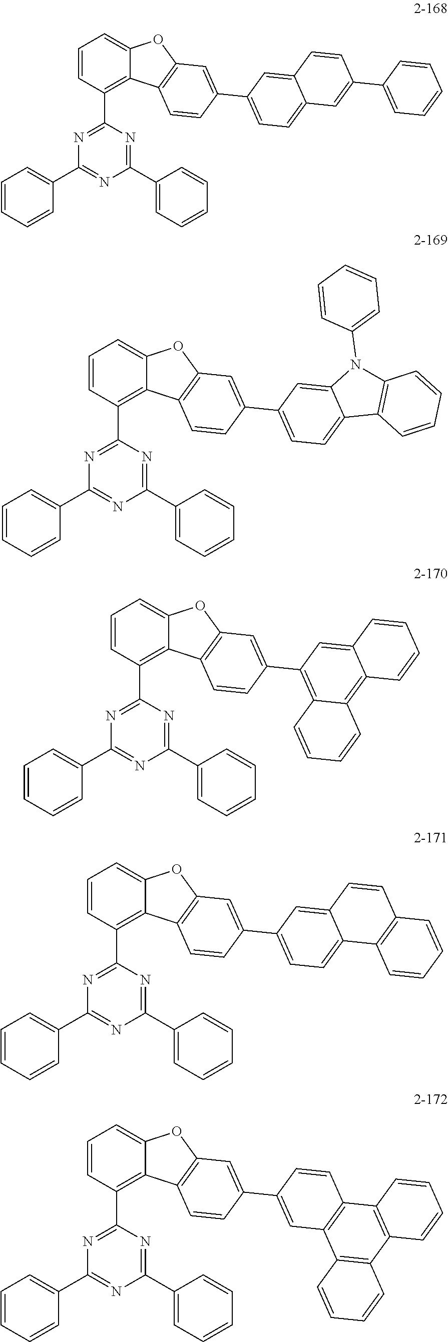 [SMILES: c1ccc(-c2ccc3cc(-c4ccc5c(c4)oc4cccc(-c6nc(-c7ccccc7)nc(-c7ccccc7)n6)c45)ccc3c2)cc1 ; c1ccc(-c2nc(-c3ccccc3)nc(-c3cccc4oc5cc(-c6ccc7c8ccccc8n(-c8ccccc8)c7c6)ccc5c34)n2)cc1 ; c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3cccc4oc5cc(-c6ccc7c8ccccc8c8ccccc8c7c6)ccc5c34)n2)cc1]

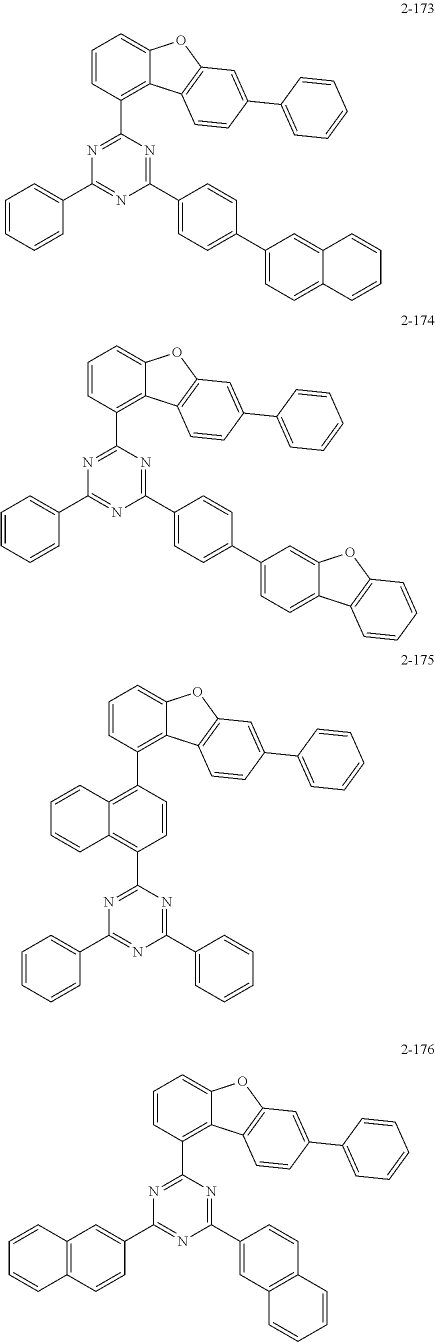 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2ccc3c(c2)oc2cccc(-c4nc(-c5ccccc5)nc(-c5ccc(-c6ccc7c(c6)oc6ccccc67)cc5)n4)c23)cc1 ; c1ccc(-c2ccc3c(c2)oc2cccc(-c4ccc(-c5nc(-c6ccccc6)nc(-c6ccccc6)n5)c5ccccc45)c23)cc1 ; c1ccc(-c2ccc3c(c2)oc2cccc(-c4nc(-c5ccc6ccccc6c5)nc(-c5ccc6ccccc6c5)n4)c23)cc1]

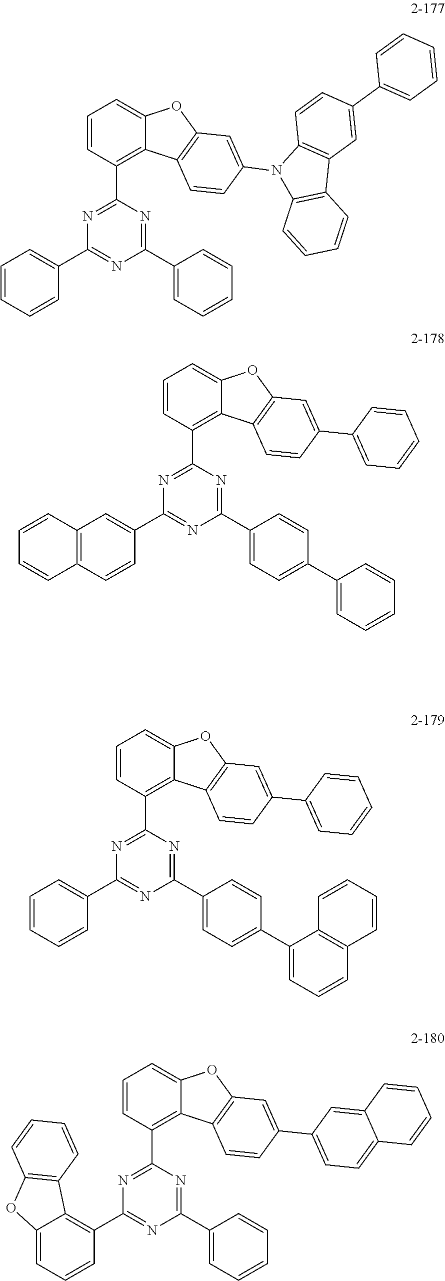 [SMILES: c1ccc(-c2ccc3c(c2)c2ccccc2n3-c2ccc3c(c2)oc2cccc(-c4nc(-c5ccccc5)nc(-c5ccccc5)n4)c23)cc1 ; c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc3c(c2)oc2cccc(-c4nc(-c5ccccc5)nc(-c5ccc(-c6cccc7ccccc67)cc5)n4)c23)cc1 ; c1ccc(-c2nc(-c3cccc4oc5ccccc5c34)nc(-c3cccc4oc5cc(-c6ccc7ccccc7c6)ccc5c34)n2)cc1]

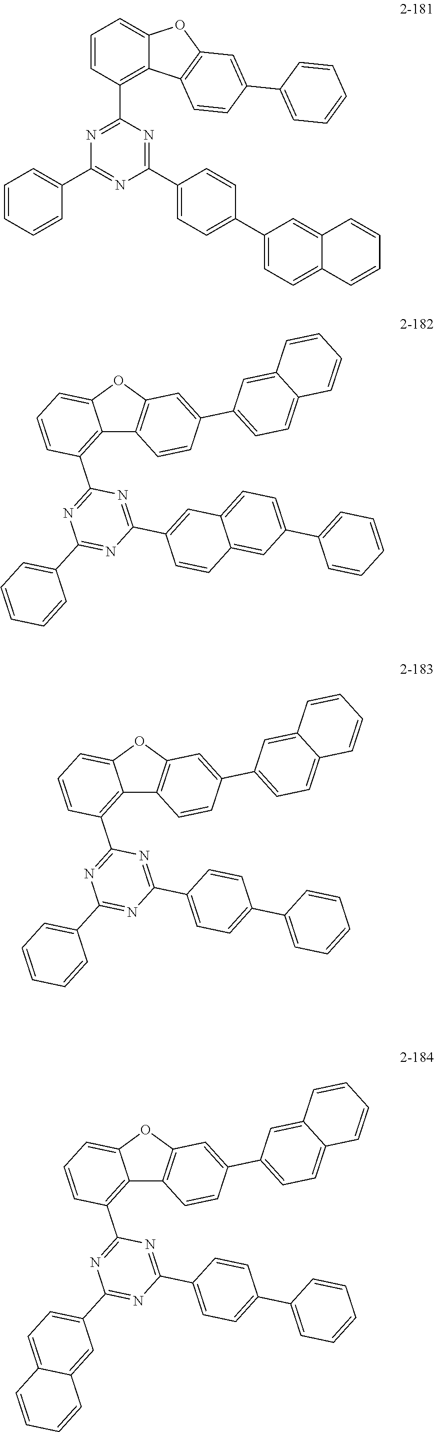 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2ccc3cc(-c4nc(-c5ccccc5)nc(-c5cccc6oc7cc(-c8ccc9ccccc9c8)ccc7c56)n4)ccc3c2)cc1 ; c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4cccc5oc6cc(-c7ccc8ccccc8c7)ccc6c45)n3)cc2)cc1]

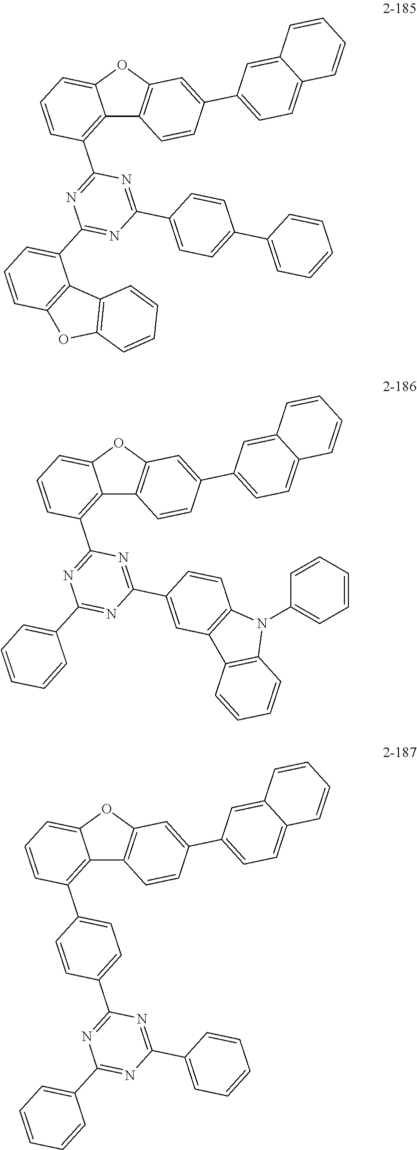 [SMILES: c1ccc(-c2ccc(-c3nc(-c4cccc5oc6ccccc6c45)nc(-c4cccc5oc6cc(-c7ccc8ccccc8c7)ccc6c45)n3)cc2)cc1 ; c1ccc(-c2nc(-c3ccc4c(c3)c3ccccc3n4-c3ccccc3)nc(-c3cccc4oc5cc(-c6ccc7ccccc7c6)ccc5c34)n2)cc1 ; c1ccc(-c2nc(-c3ccccc3)nc(-c3ccc(-c4cccc5oc6cc(-c7ccc8ccccc8c7)ccc6c45)cc3)n2)cc1]

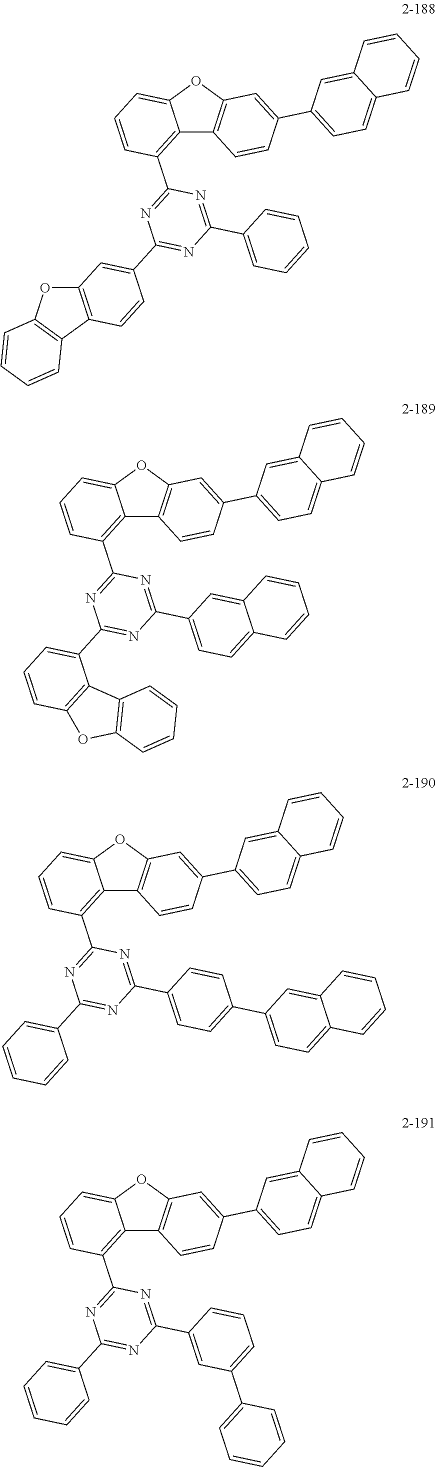 [SMILES: c1ccc(-c2nc(-c3ccc4c(c3)oc3ccccc34)nc(-c3cccc4oc5cc(-c6ccc7ccccc7c6)ccc5c34)n2)cc1 ; c1ccc2cc(-c3ccc4c(c3)oc3cccc(-c5nc(-c6ccc7ccccc7c6)nc(-c6cccc7oc8ccccc8c67)n5)c34)ccc2c1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2cccc(-c3nc(-c4ccccc4)nc(-c4cccc5oc6cc(-c7ccc8ccccc8c7)ccc6c45)n3)c2)cc1]

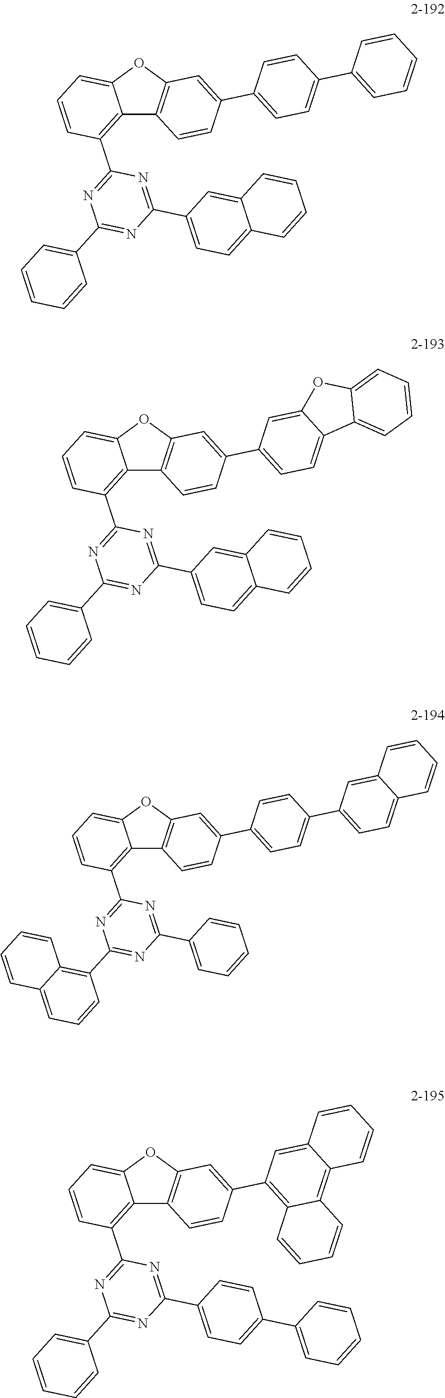 [SMILES: c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2nc(-c3ccc4ccccc4c3)nc(-c3cccc4oc5cc(-c6ccc7c(c6)oc6ccccc67)ccc5c34)n2)cc1 ; c1ccc(-c2nc(-c3cccc4ccccc34)nc(-c3cccc4oc5cc(-c6ccc(-c7ccc8ccccc8c7)cc6)ccc5c34)n2)cc1 ; c1ccc(-c2ccc(-c3nc(-c4ccccc4)nc(-c4cccc5oc6cc(-c7cc8ccccc8c8ccccc78)ccc6c45)n3)cc2)cc1]

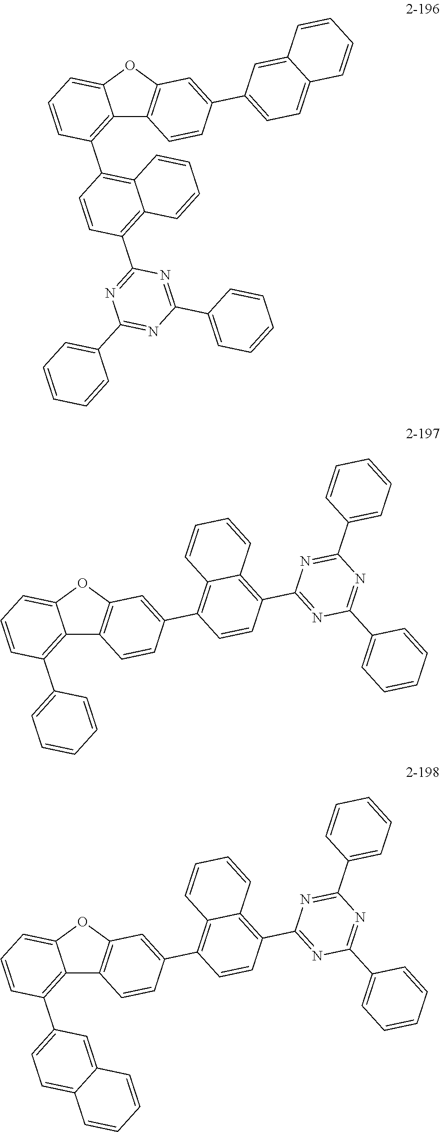 [SMILES: c1ccc(-c2nc(-c3ccccc3)nc(-c3ccc(-c4cccc5oc6cc(-c7ccc8ccccc8c7)ccc6c45)c4ccccc34)n2)cc1 ; c1ccc(-c2nc(-c3ccccc3)nc(-c3ccc(-c4ccc5c(c4)oc4cccc(-c6ccccc6)c45)c4ccccc34)n2)cc1 ; c1ccc(-c2nc(-c3ccccc3)nc(-c3ccc(-c4ccc5c(c4)oc4cccc(-c6ccc7ccccc7c6)c45)c4ccccc34)n2)cc1]

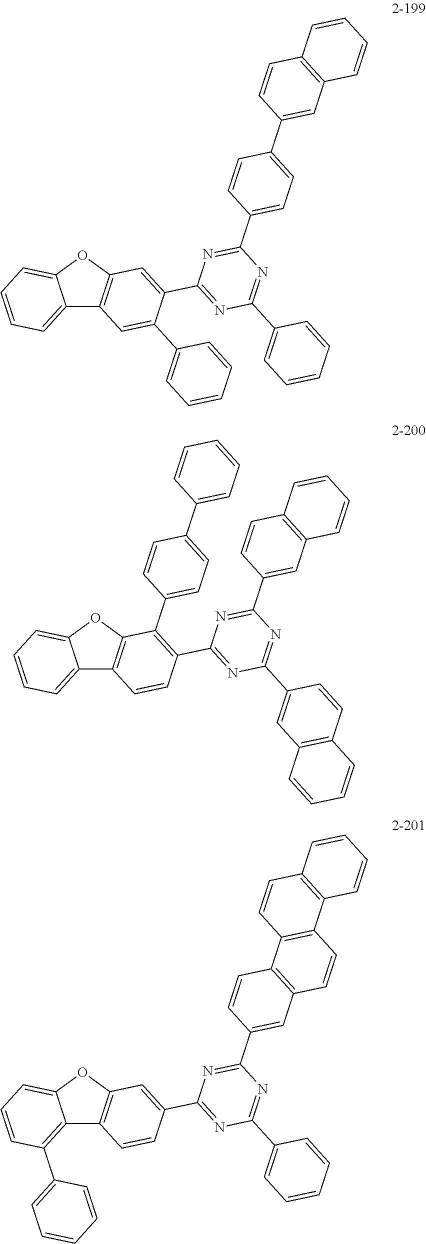

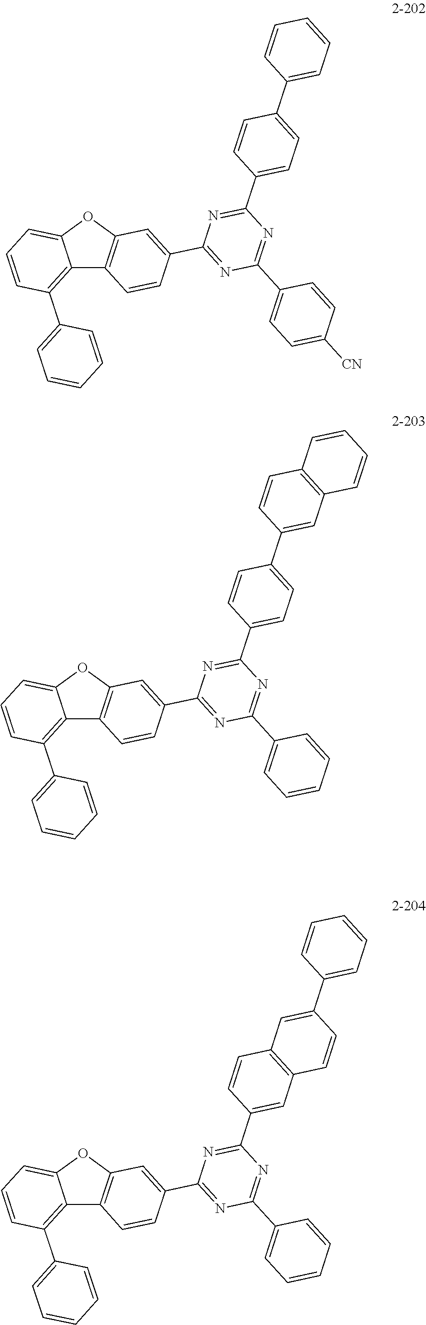 [SMILES: N#Cc1ccc(-c2nc(-c3ccc(-c4ccccc4)cc3)nc(-c3ccc4c(c3)oc3cccc(-c5ccccc5)c34)n2)cc1 ; c1ccc(-c2nc(-c3ccc(-c4ccc5ccccc5c4)cc3)nc(-c3ccc4c(c3)oc3cccc(-c5ccccc5)c34)n2)cc1 ; c1ccc(-c2ccc3cc(-c4nc(-c5ccccc5)nc(-c5ccc6c(c5)oc5cccc(-c7ccccc7)c56)n4)ccc3c2)cc1]

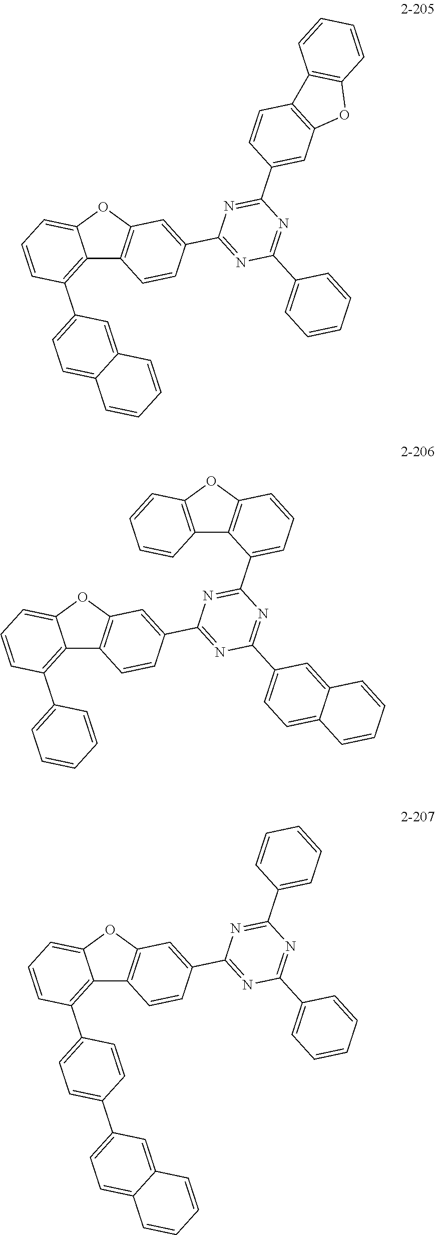 [SMILES: c1ccc(-c2nc(-c3ccc4c(c3)oc3ccccc34)nc(-c3ccc4c(c3)oc3cccc(-c5ccc6ccccc6c5)c34)n2)cc1 ; c1ccc(-c2cccc3oc4cc(-c5nc(-c6ccc7ccccc7c6)nc(-c6cccc7oc8ccccc8c67)n5)ccc4c23)cc1 ; c1ccc(-c2nc(-c3ccccc3)nc(-c3ccc4c(c3)oc3cccc(-c5ccc(-c6ccc7ccccc7c6)cc5)c34)n2)cc1]

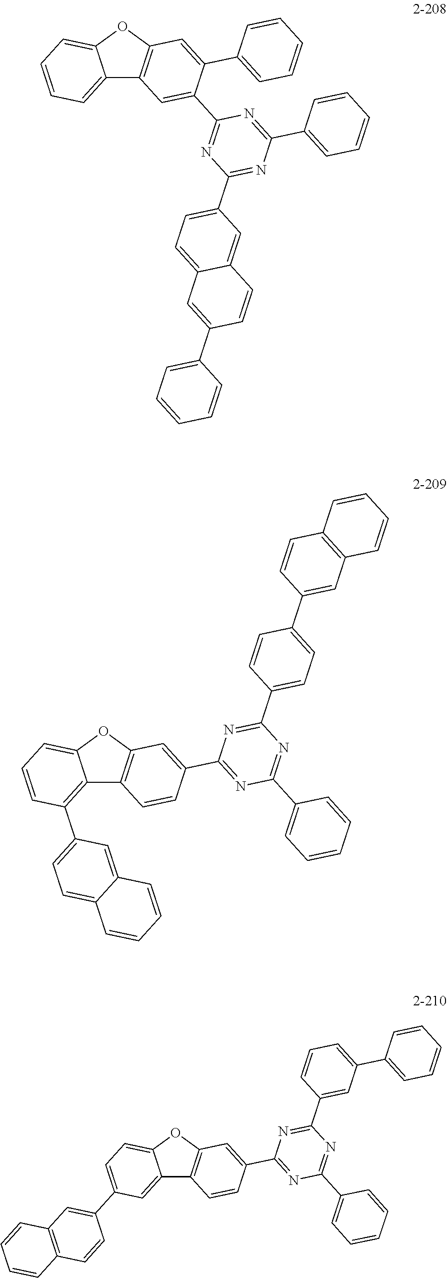 [SMILES: c1ccc(-c2ccc3cc(-c4nc(-c5ccccc5)nc(-c5cc6c(cc5-c5ccccc5)oc5ccccc56)n4)ccc3c2)cc1 ; c1ccc(-c2nc(-c3ccc(-c4ccc5ccccc5c4)cc3)nc(-c3ccc4c(c3)oc3cccc(-c5ccc6ccccc6c5)c34)n2)cc1 ; c1ccc(-c2cccc(-c3nc(-c4ccccc4)nc(-c4ccc5c(c4)oc4ccc(-c6ccc7ccccc7c6)cc45)n3)c2)cc1]

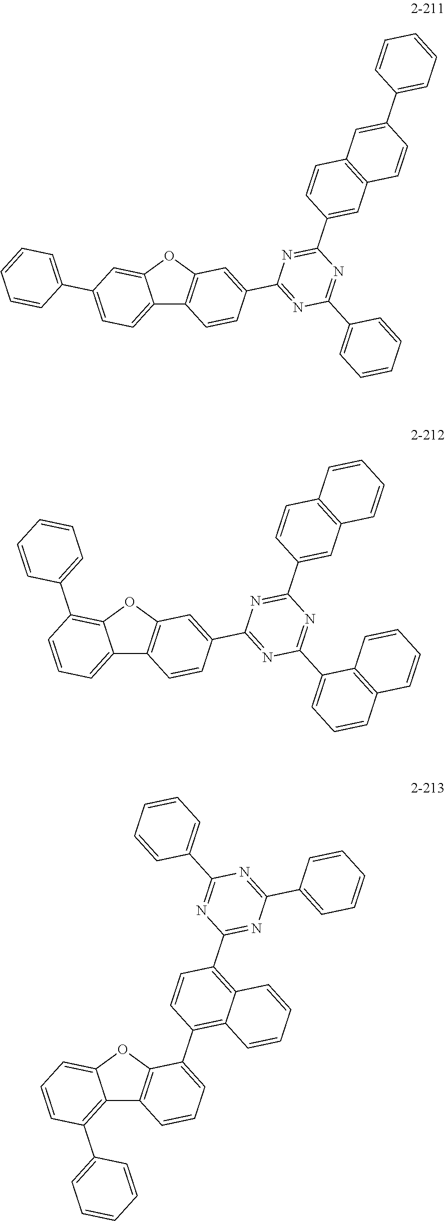 [SMILES: c1ccc(-c2ccc3cc(-c4nc(-c5ccccc5)nc(-c5ccc6c(c5)oc5cc(-c7ccccc7)ccc56)n4)ccc3c2)cc1 ; c1ccc(-c2cccc3c2oc2cc(-c4nc(-c5ccc6ccccc6c5)nc(-c5cccc6ccccc56)n4)ccc23)cc1 ; c1ccc(-c2nc(-c3ccccc3)nc(-c3ccc(-c4cccc5c4oc4cccc(-c6ccccc6)c45)c4ccccc34)n2)cc1]

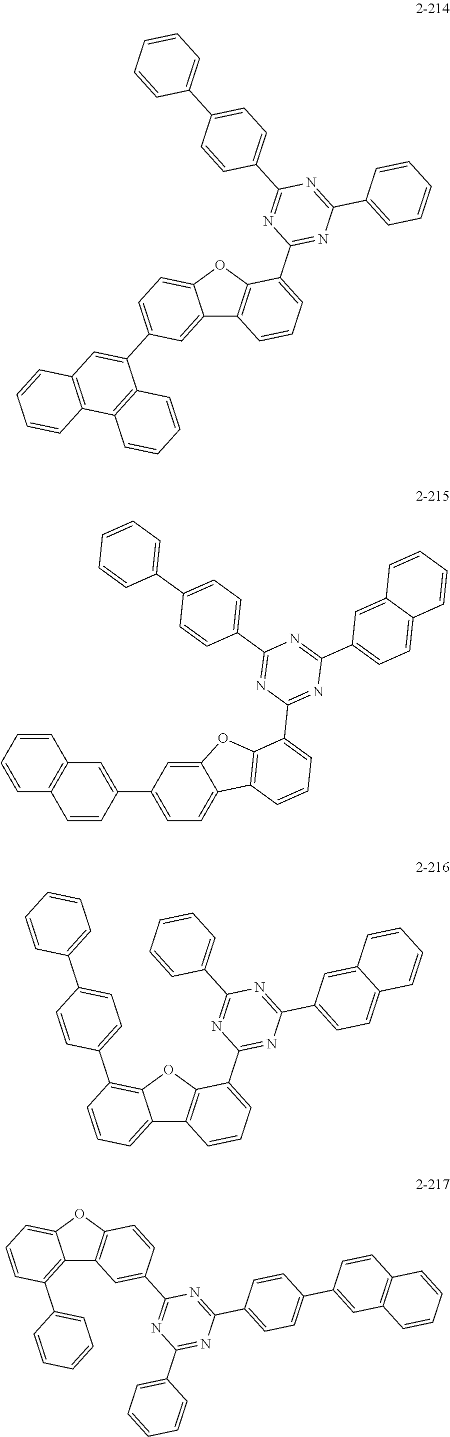 [SMILES: c1ccc(-c2ccc(-c3nc(-c4ccccc4)nc(-c4cccc5c4oc4ccc(-c6cc7ccccc7c7ccccc67)cc45)n3)cc2)cc1 ; c1ccc(-c2ccc(-c3nc(-c4ccc5ccccc5c4)nc(-c4cccc5c4oc4cc(-c6ccc7ccccc7c6)ccc45)n3)cc2)cc1 ; c1ccc(-c2ccc(-c3cccc4c3oc3c(-c5nc(-c6ccccc6)nc(-c6ccc7ccccc7c6)n5)cccc34)cc2)cc1 ; c1ccc(-c2nc(-c3ccc(-c4ccc5ccccc5c4)cc3)nc(-c3ccc4oc5cccc(-c6ccccc6)c5c4c3)n2)cc1]

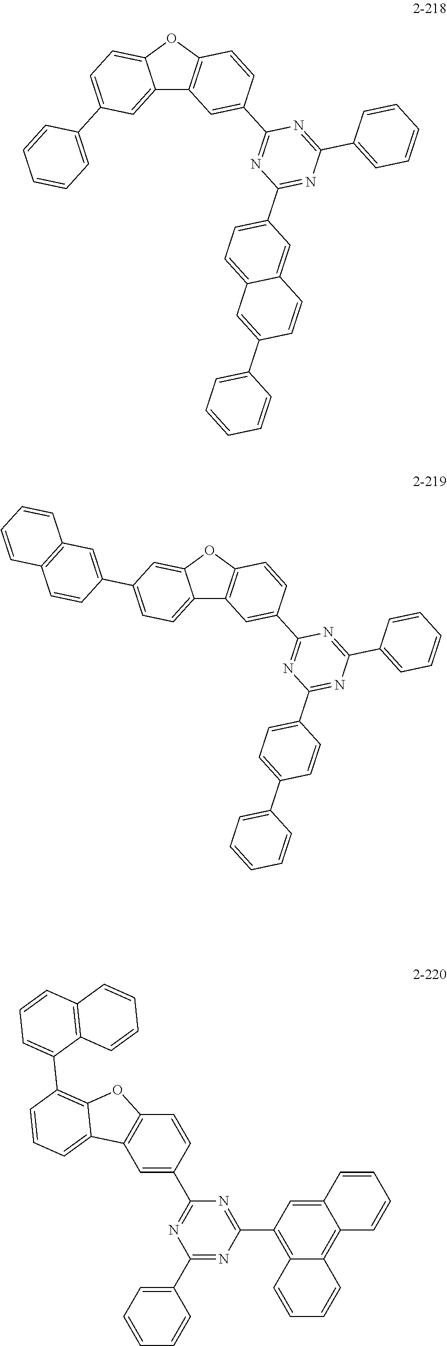 [SMILES: c1ccc(-c2ccc3cc(-c4nc(-c5ccccc5)nc(-c5ccc6oc7ccc(-c8ccccc8)cc7c6c5)n4)ccc3c2)cc1 ; c1ccc(-c2ccc(-c3nc(-c4ccccc4)nc(-c4ccc5oc6cc(-c7ccc8ccccc8c7)ccc6c5c4)n3)cc2)cc1 ; c1ccc(-c2nc(-c3ccc4oc5c(-c6cccc7ccccc67)cccc5c4c3)nc(-c3cc4ccccc4c4ccccc34)n2)cc1]

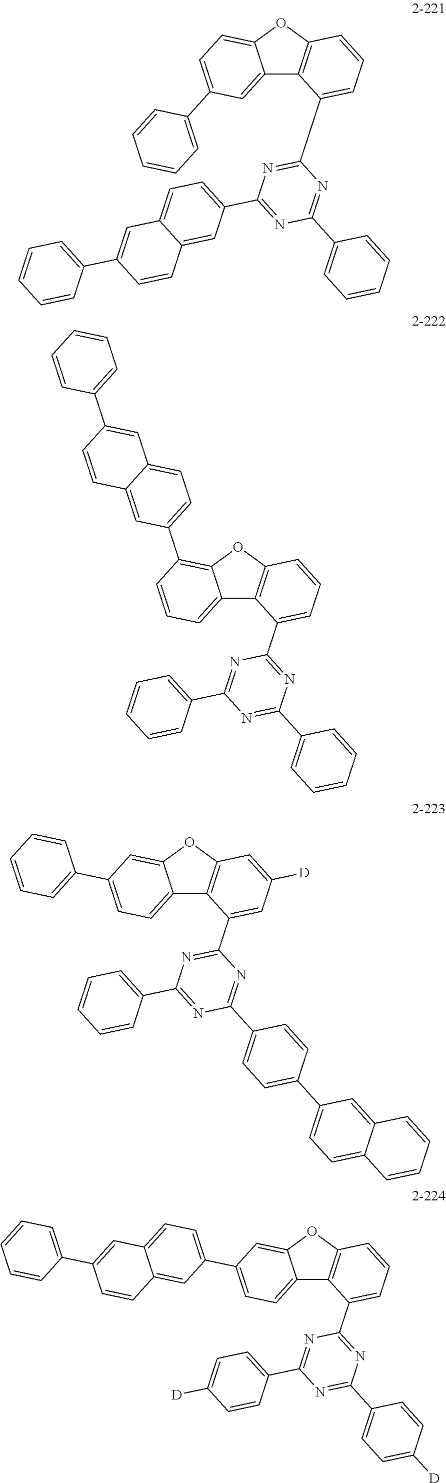

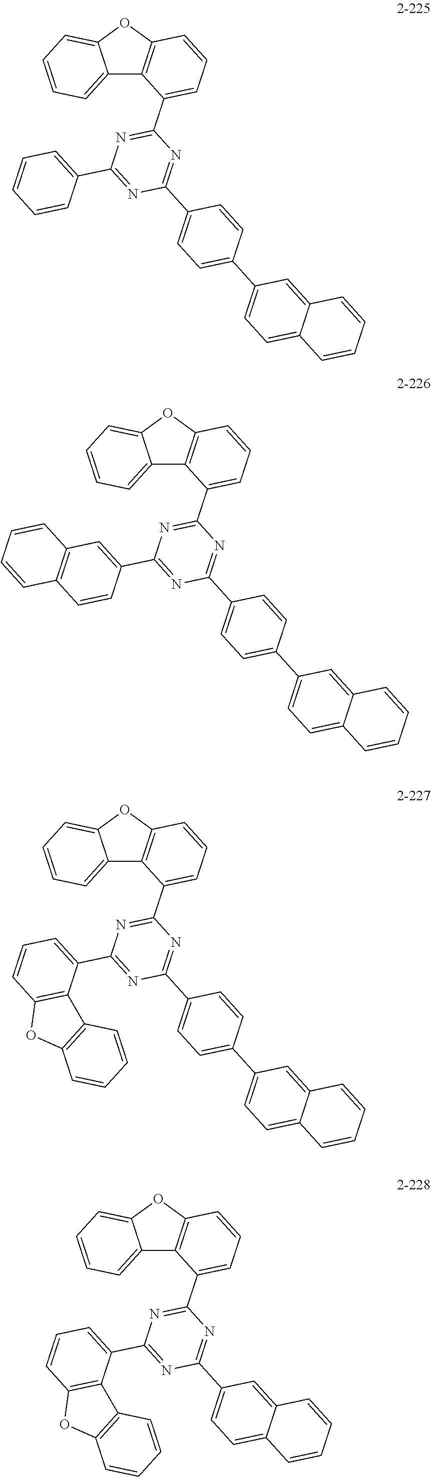 [SMILES: c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc2cc(-c3ccc(-c4nc(-c5cccc6oc7ccccc7c56)nc(-c5cccc6oc7ccccc7c56)n4)cc3)ccc2c1 ; c1ccc2cc(-c3nc(-c4cccc5oc6ccccc6c45)nc(-c4cccc5oc6ccccc6c45)n3)ccc2c1]

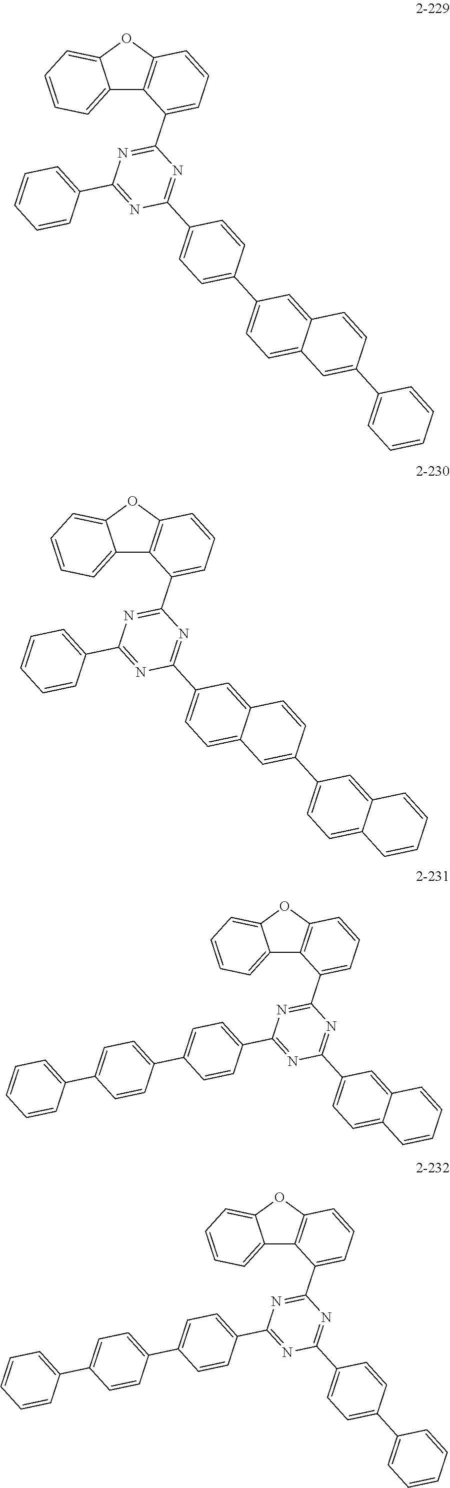 [SMILES: c1ccc(-c2ccc3cc(-c4ccc(-c5nc(-c6ccccc6)nc(-c6cccc7oc8ccccc8c67)n5)cc4)ccc3c2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3ccc(-c4nc(-c5ccc(-c6ccccc6)cc5)nc(-c5cccc6oc7ccccc7c56)n4)cc3)cc2)cc1]

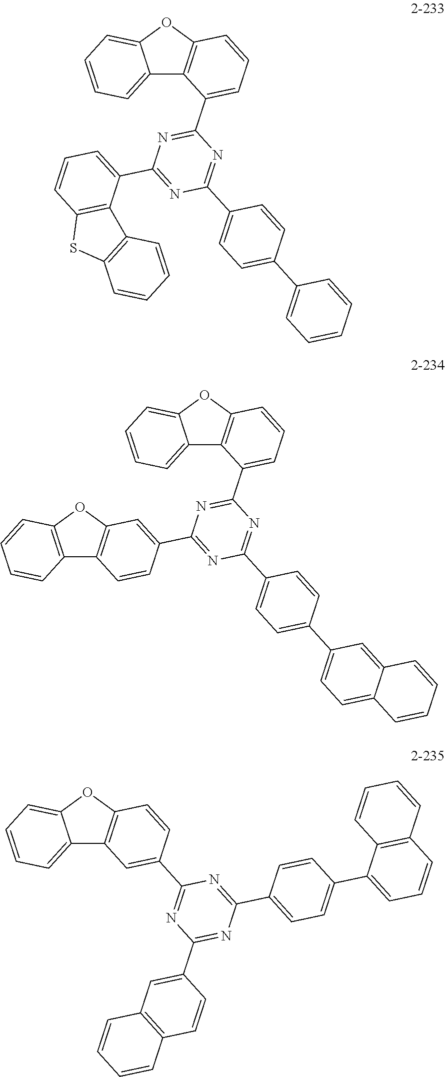 [SMILES: c1ccc(-c2ccc(-c3nc(-c4cccc5oc6ccccc6c45)nc(-c4cccc5sc6ccccc6c45)n3)cc2)cc1 ; c1ccc2cc(-c3ccc(-c4nc(-c5ccc6c(c5)oc5ccccc56)nc(-c5cccc6oc7ccccc7c56)n4)cc3)ccc2c1 ; c1ccc2cc(-c3nc(-c4ccc(-c5cccc6ccccc56)cc4)nc(-c4ccc5oc6ccccc6c5c4)n3)ccc2c1]

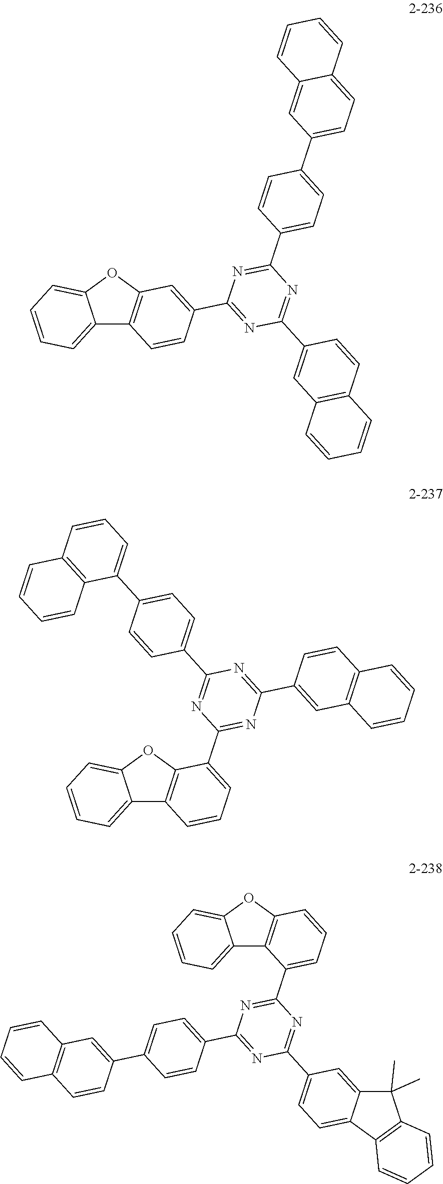 [SMILES: c1ccc2cc(-c3ccc(-c4nc(-c5ccc6ccccc6c5)nc(-c5ccc6c(c5)oc5ccccc56)n4)cc3)ccc2c1 ; c1ccc2cc(-c3nc(-c4ccc(-c5cccc6ccccc56)cc4)nc(-c4cccc5c4oc4ccccc45)n3)ccc2c1 ; CC1(C)c2ccccc2-c2ccc(-c3nc(-c4ccc(-c5ccc6ccccc6c5)cc4)nc(-c4cccc5oc6ccccc6c45)n3)cc21]

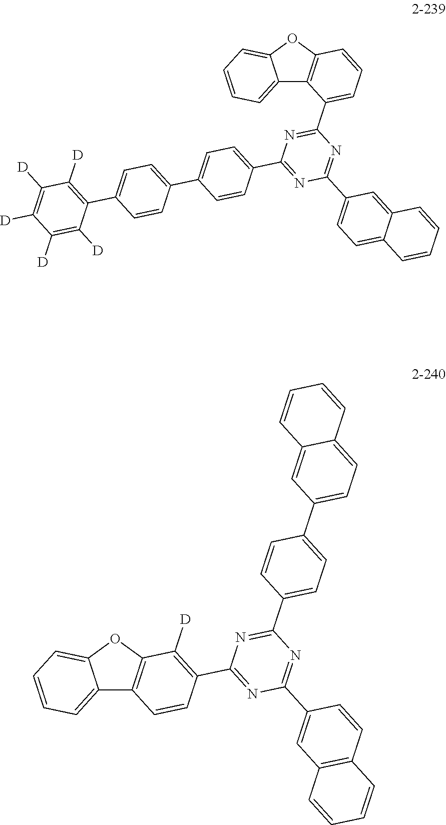 [SMILES: [2H]c1c([2H])c([2H])c(-c2ccc(-c3ccc(-c4nc(-c5ccc6ccccc6c5)nc(-c5cccc6oc7ccccc7c56)n4)cc3)cc2)c([2H])c1[2H] ; [2H]c1c(-c2nc(-c3ccc(-c4ccc5ccccc5c4)cc3)nc(-c3ccc4ccccc4c3)n2)ccc2c1oc1ccccc12]

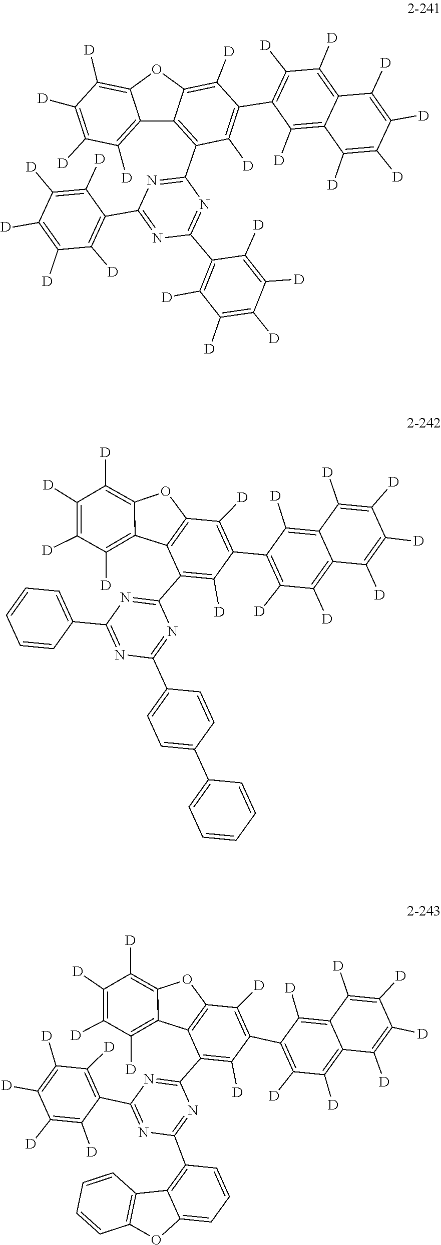

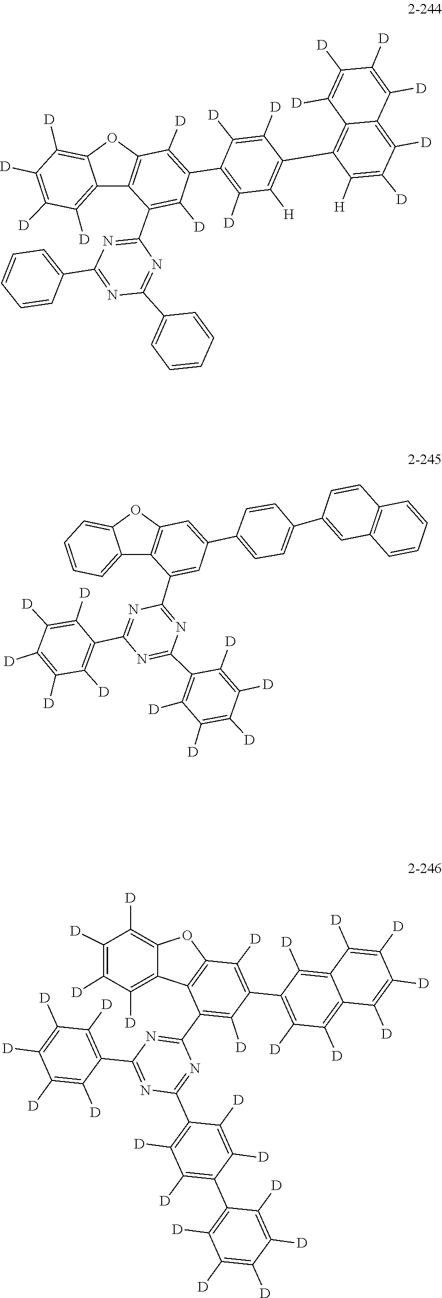

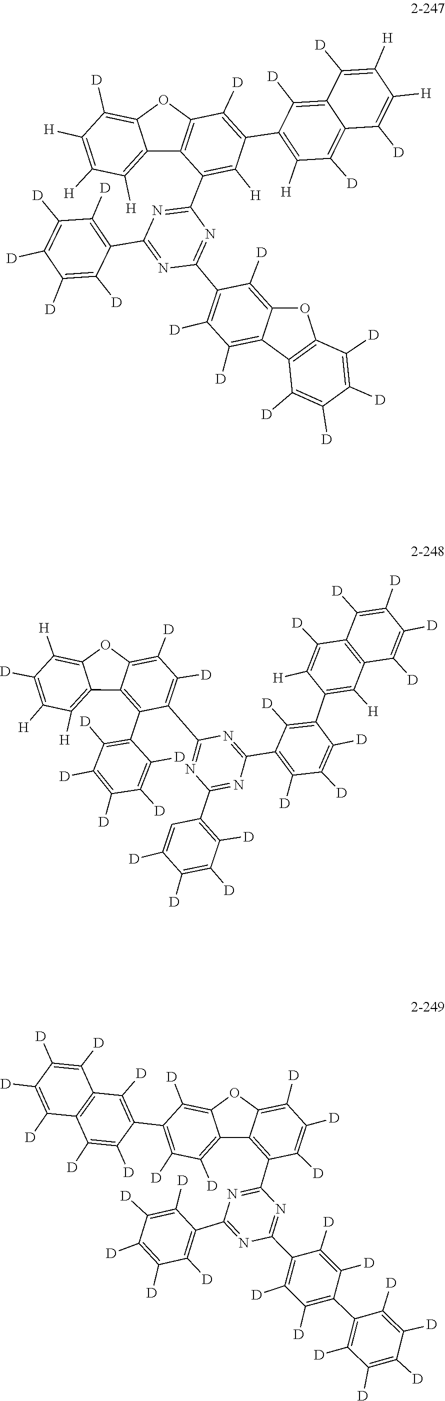

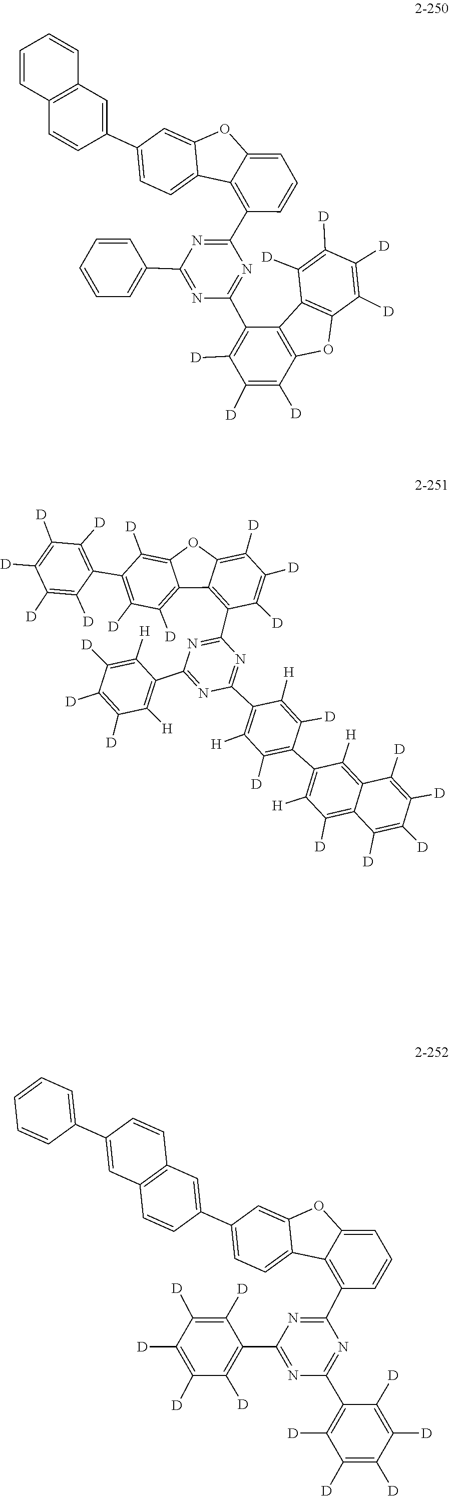 [SMILES: [2H]c1c([2H])c([2H])c2c(oc3c([2H])c([2H])c([2H])c(-c4nc(-c5ccccc5)nc(-c5cccc6oc7cc(-c8ccc9ccccc9c8)ccc7c56)n4)c32)c1[2H] ; [2H]c1cc(-c2nc(-c3cc([2H])c(-c4cc([2H])c5c([2H])c([2H])c([2H])c([2H])c5c4)c([2H])c3)nc(-c3c([2H])c([2H])c([2H])c4oc5c([2H])c(-c6c([2H])c([2H])c([2H])c([2H])c6[2H])c([2H])c([2H])c5c34)n2)cc([2H])c1[2H] ; [2H]c1c([2H])c([2H])c(-c2nc(-c3c([2H])c([2H])c([2H])c([2H])c3[2H])nc(-c3cccc4oc5cc(-c6ccc7cc(-c8ccccc8)ccc7c6)ccc5c34)n2)c([2H])c1[2H]]

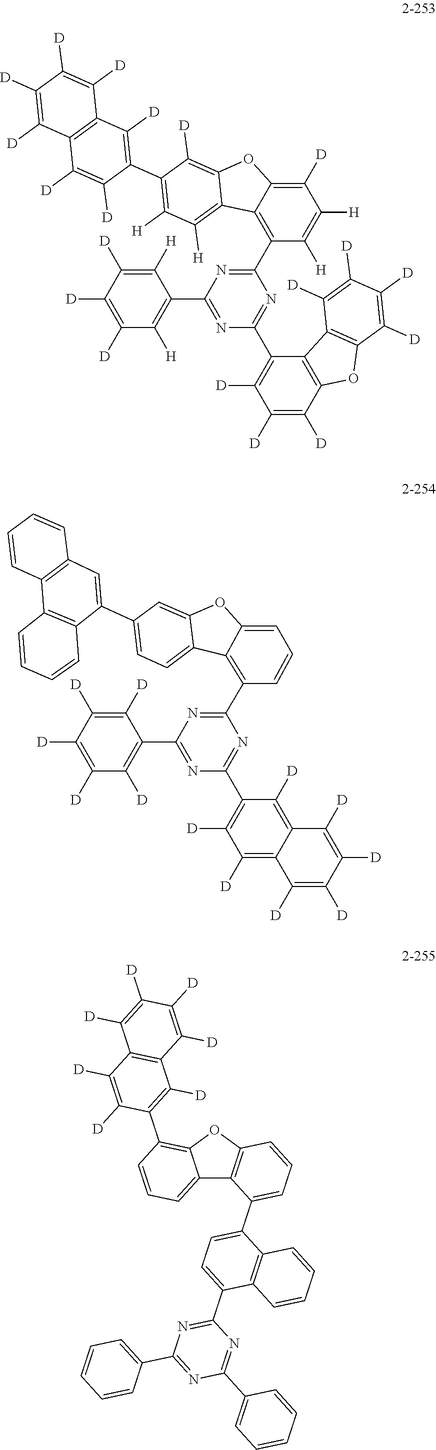 [SMILES: [2H]c1cc(-c2nc(-c3ccc([2H])c4oc5c([2H])c(-c6c([2H])c([2H])c7c([2H])c([2H])c([2H])c([2H])c7c6[2H])ccc5c34)nc(-c3c([2H])c([2H])c([2H])c4oc5c([2H])c([2H])c([2H])c([2H])c5c34)n2)cc([2H])c1[2H] ; [2H]c1c([2H])c([2H])c(-c2nc(-c3c([2H])c([2H])c4c([2H])c([2H])c([2H])c([2H])c4c3[2H])nc(-c3cccc4oc5cc(-c6cc7ccccc7c7ccccc67)ccc5c34)n2)c([2H])c1[2H] ; [2H]c1c([2H])c([2H])c2c([2H])c(-c3cccc4c3oc3cccc(-c5ccc(-c6nc(-c7ccccc7)nc(-c7ccccc7)n6)c6ccccc56)c34)c([2H])c([2H])c2c1[2H]]

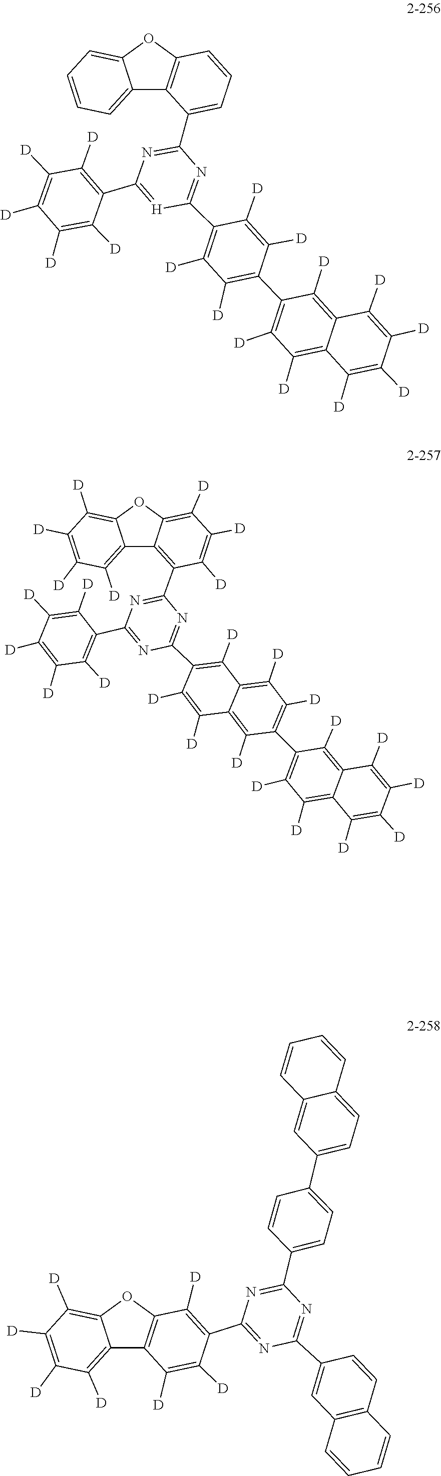

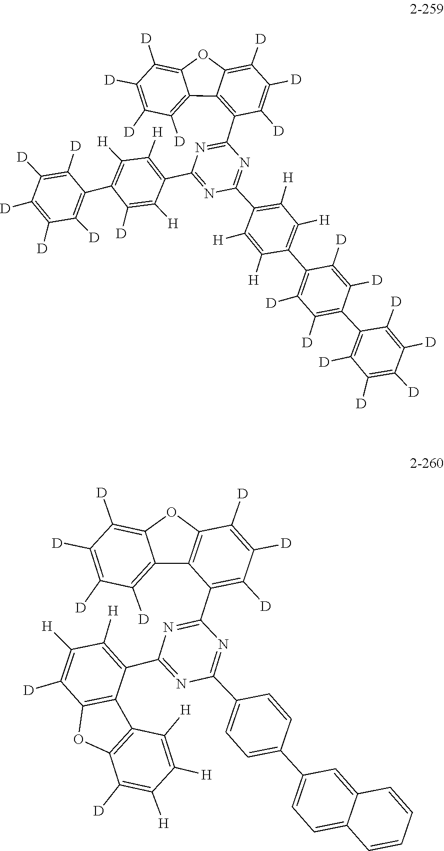 [SMILES: [2H]c1cc(-c2nc(-c3ccc(-c4c([2H])c([2H])c(-c5c([2H])c([2H])c([2H])c([2H])c5[2H])c([2H])c4[2H])cc3)nc(-c3c([2H])c([2H])c([2H])c4oc5c([2H])c([2H])c([2H])c([2H])c5c34)n2)ccc1-c1c([2H])c([2H])c([2H])c([2H])c1[2H] ; [2H]c1c([2H])c([2H])c2c(oc3c([2H])c([2H])c([2H])c(-c4nc(-c5ccc(-c6ccc7ccccc7c6)cc5)nc(-c5ccc([2H])c6oc7c([2H])cccc7c56)n4)c32)c1[2H]]

When the organic layer 300 includes the compound represented by Formula 3 together with the heterocyclic compound, the electron and hole transport characteristics of the light-emitting device may be further enhanced, and the light-emitting efficiency and device lifetime may be further improved.

In one embodiment, the compound represented by Formula 3 may be included in the same organic layer as the heterocyclic compound, and for example, may be included in the light-emitting layer 303. For example, the heterocyclic compound represented by Formula 1 may be a p-type host material, and the compound represented by Formula 3 may be an n-type host material.

For example, the heterocyclic compound represented by Formula 1 may have high hole transport ability, thereby acting as a donor, and the compound represented by Formula 3 may have a high electron transport ability, thereby acting as an acceptor. When these compounds are used together, an exciplex phenomenon may occur, thereby improving charge balance within the device.

In one embodiment, the weight ratio of the heterocyclic compound to the compound represented by Formula 3 in the organic layer 300 or the light-emitting layer 303 may be 1:10 to 10:1, 1:8 to 8:1, 1:5 to 5:1, or 1:2 to 2:1. Within this range, both electron transport characteristics and hole transport characteristics in the organic layer 300 may be improved. Accordingly, the operating voltage of the light-emitting device may be lowered, and the light-emitting efficiency and lifetime characteristics may be further improved.

As the material of the hole injection layer 301, known hole injection layer materials may be used. For example, phthalocyanine compounds such as copper phthalocyanine disclosed in U.S. Pat. No. 4,356,429, or a starburst amine derivative described in the literature [Advanced Materials, 6, p. 677 (1994)], such as tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), 4,4′,4″-tris(N,N-(2-naphthyl)-phenylamino)triphenylamine (2-TNATA), or a soluble conductive polymer such as polyaniline/dodecylbenzenesulfonic acid, poly(3,4-ethylenedioxythiophene)/poly(4-styrenesulfonate), polyaniline/camphorsulfonic acid, or polyaniline/poly(4-styrenesulfonate) may be used.

As the materials of the hole transport layer 302, pyrazoline derivatives, arylamine derivatives, stilbene derivatives, or triphenyldiamine derivatives may be used, and low-molecular-weight or high-molecular-weight materials may also be used. As the materials of the electron transport layer, oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, or 8-hydroxyquinoline and metal complexes thereof may be used. Not only low-molecular-weight materials but also high-molecular-weight materials may be used.

For example, LiF is typically used in the art as the material of the electron injection layer 305, but the present application is not limited thereto.

As the materials of the electron transport layer 304, anthracene compounds, Alq₃ (tris(8-hydroxyquinolinato)aluminum), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, TPBi (1,3,5-tri(1-phenyl-1H-benzo[d]imidazol-2-yl)benzene), BCP (2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4-(naphthalen-1-yl)-3,5-diphenyl-4H-1,2,4-triazole), tBu-PBD (2-(4-biphenylyl)-5-(4-tert-butylphenyl)-1,3,4-oxadiazole), BAlq (bis(2-methyl-8-quinolinolato-N1,O8)-(1,1′-biphenyl-4-olato)aluminum), Bebg2 (beryllium bis(benzoquinolin-10-olate)), ADN(9,10-di(naphthalene-2-yl)anthracene), BmPyPhB (1,3-bis[3,5-di(pyridin-3-yl)phenyl]benzene), or a combination thereof may beused.

As used herein, the charge generation layer may be an N-type charge generation layer, and may further include a dopant known in the art.

According to exemplary embodiments, a method for manufacturing the light-emitting device may be provided.

According to exemplary embodiments, a substrate may be prepared. A first electrode may be formed on the substrate. One or more organic layers may be formed on the first electrode. A second electrode may be formed on the organic layer to manufacture alight-emitting device.

The light-emitting device may be manufactured using conventional methods and materials for manufacturing a light-emitting device, except that one or more organic layers are formed using the above-described heterocyclic compound.

When manufacturing an organic light-emitting device, an organic layer including the heterocyclic compound may be formed using a vacuum deposition method. Alternatively, the organic layer may be formed by applying a composition for an organic layer including the heterocyclic compound using a solution application method. Here, the solution application method may include spin coating dip coating inkjet printing screen printing spray coating or roll coating but is not limited thereto.

The organic layer may be formed using a composition for an organic layer including the heterocyclic compound represented by Formula 1.

The composition for an organic layer of an organic light-emitting device according to the present disclosure may include a heterocyclic compound represented by Formula 1 and a compound represented by Formula 3. The two compounds may function as a dual host.

The organic layer may be formed by pre-mixing the heterocyclic compound represented by Formula 1 and the compound represented by Formula 3 using a thermal vacuum deposition method.

The ratio of the weight of the compound represented by Formula 3 to the weight of the heterocyclic compound represented by Formula 1, based on the total weight of the composition for an organic layer, may be 0.1 to 10. In some embodiments, the ratio of the weight of the compound represented by Formula 3 to the weight of the heterocyclic compound represented by Formula 1, based on the total weight of the composition for an organic layer, may be 0.2 to 5, or 0.5 to 2.

The composition may be used in forming the organic layer of an organic light-emitting device, and in particular, preferably used as a host material for the light-emitting layer.

The composition is in a form in which two or more compounds are simply mixed, and may be prepared by mixing powdered materials prior to forming the organic layer of an organic light-emitting device, or by mixing compounds that are liquid at a temperature above a predetermined temperature. The composition is in a solid state at or below the melting point of each material and may be maintained in a liquid state under high-temperature conditions.

The composition may further include materials known in the art, such as solvents and additives.

Hereinafter, embodiments of the present invention will be further described with reference to specific experimental examples. The examples and comparative examples included in the experimental examples are merely illustrative of the present disclosure and do not limit the scope of the appended claims. It will be apparent to those skilled in the art that various changes and modifications to the examples can be made within the scope and technical spirit of the present disclosure, and it is also understood that such changes and modifications fall within the scope of the appended claims.

### Preparative Example 1: Preparation of Compound 1-12

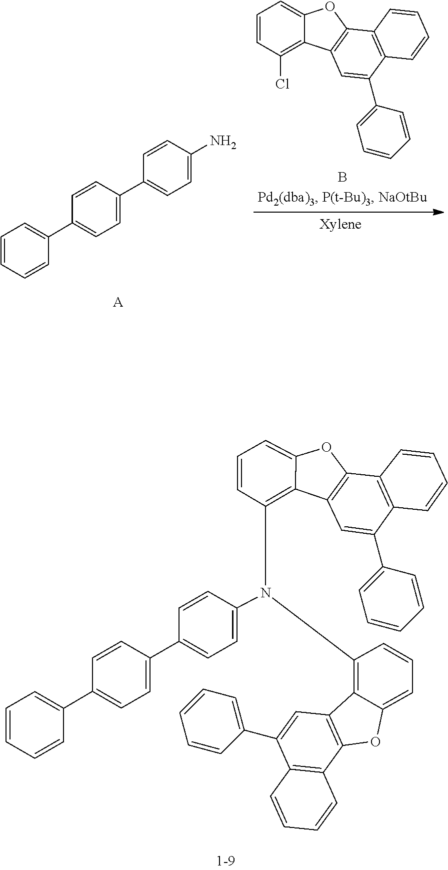

[1,1′:4′,1″-terphenyl]-4-amine (A, 3.5 g 14.27 mmol), 7-chloro-5-phenylnaphtho[1,2-b]benzofuran (B, 10.32 g 31.39 mmol), Pd₂(dba)₃ (0.52 g 0.57 mmol), NaOtBu (2.74 g 28.53 mmol), and xylene (35 mL) were introduced into a 100 mL round-bottom flask, followed by the addition of P(t-Bu)₃ (0.56 mL, 1.14 mmol), and the reaction mixture was refluxed at 140° C. for 12 hours. After completion of the reaction, the reaction temperature was lowered to room temperature, washed with water, and extracted with DCM. The organic layer was dried over Mg₂SO₄ and concentrated under reduced pressure to remove the solvent. The residue was purified by silica gel column chromatography and recrystallized to obtain Compound 1-12 (8.56 g 10.28 mmol, 72%) as a solid.

The target compound was prepared in the same manner as in Preparative Example 1, except that Compound A shown in Table 1 was used instead of [1,1′:4′,1″-terphenyl]-4-amine and Compound B shown in Table 1 was used instead of 7-chloro-5-phenylnaphtho[1,2-b]benzofuran, as shown in Table 1 below.

TABLE 1

| Compound No. | A | B | Target compound | Yield |
|---|---|---|---|---|
| 1-9 | 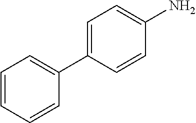 | 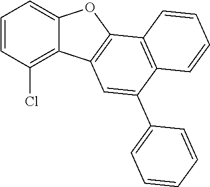 | 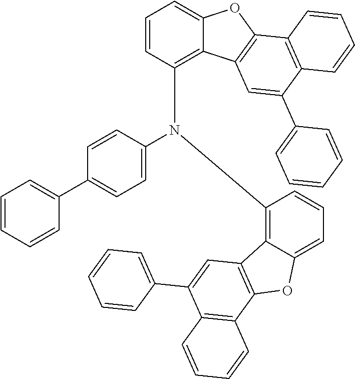 [SMILES: c1ccc(-c2ccc(N(c3cccc4oc5c6ccccc6c(-c6ccccc6)cc5c34)c3cccc4oc5c6ccccc6c(-c6ccccc6)cc5c34)cc2)cc1] | 76.3% |
| 1-12 | 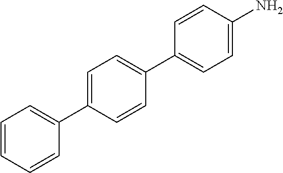 | | 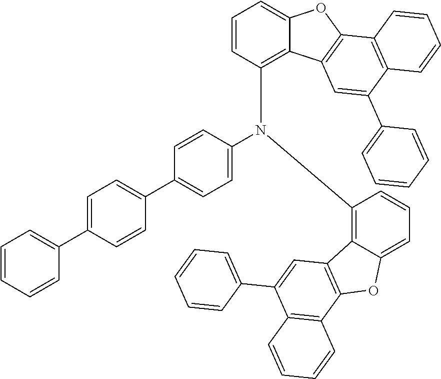 [SMILES: c1ccc(-c2ccc(-c3ccc(N(c4cccc5oc6c7ccccc7c(-c7ccccc7)cc6c45)c4cccc5oc6c7ccccc7c(-c7ccccc7)cc6c45)cc3)cc2)cc1] | 72.0% |
| 1-20 | 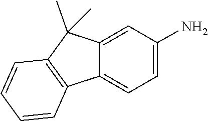 | | 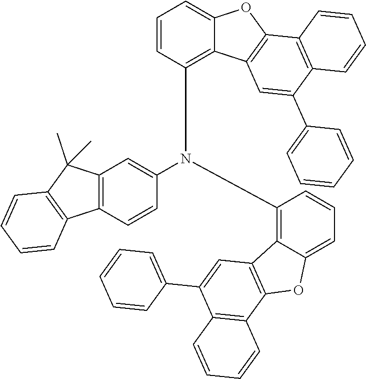 [SMILES: CC1(C)c2ccccc2-c2ccc(N(c3cccc4oc5c6ccccc6c(-c6ccccc6)cc5c34)c3cccc4oc5c6ccccc6c(-c6ccccc6)cc5c34)cc21] | 72.0% |
| 1-29 | 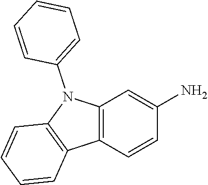 | | 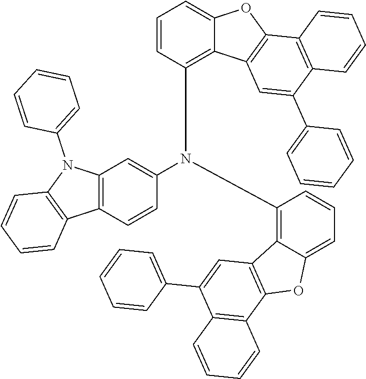 [SMILES: c1ccc(-c2cc3c(oc4cccc(N(c5ccc6c7ccccc7n(-c7ccccc7)c6c5)c5cccc6oc7c8ccccc8c(-c8ccccc8)cc7c56)c43)c3ccccc23)cc1] | 65.8% |
| 1-45 | 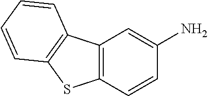 | | 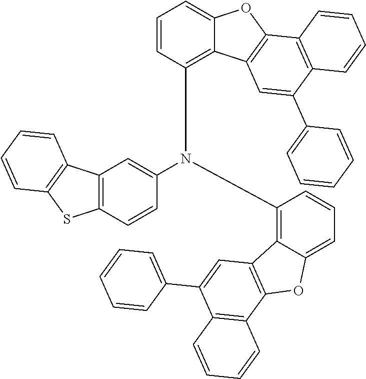 [SMILES: c1ccc(-c2cc3c(oc4cccc(N(c5ccc6sc7ccccc7c6c5)c5cccc6oc7c8ccccc8c(-c8ccccc8)cc7c56)c43)c3ccccc23)cc1] | 68.3% |
| 1-48 | 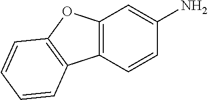 | | 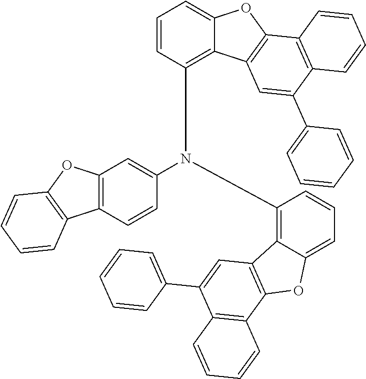 [SMILES: c1ccc(-c2cc3c(oc4cccc(N(c5ccc6c(c5)oc5ccccc56)c5cccc6oc7c8ccccc8c(-c8ccccc8)cc7c56)c43)c3ccccc23)cc1] | 85.9% |
| 1-117 | 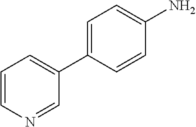 | 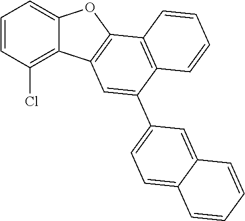 | 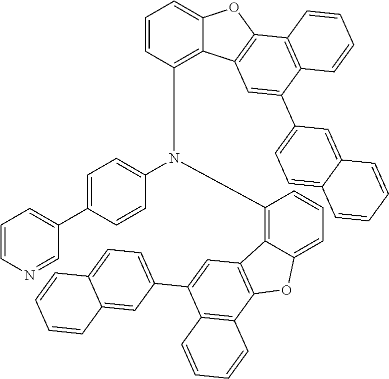 [SMILES: c1cncc(-c2ccc(N(c3cccc4oc5c6ccccc6c(-c6ccc7ccccc7c6)cc5c34)c3cccc4oc5c6ccccc6c(-c6ccc7ccccc7c6)cc5c34)cc2)c1] | 75.4% |
| 1-23 | 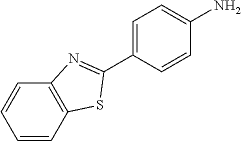 | | 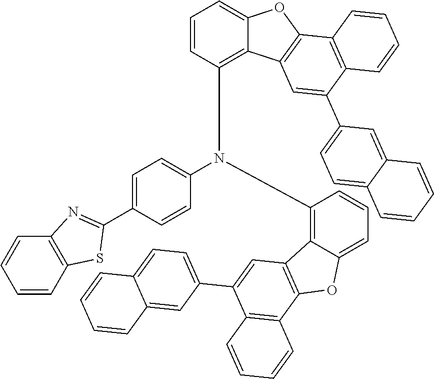 [SMILES: c1ccc2cc(-c3cc4c(oc5cccc(N(c6ccc(-c7nc8ccccc8s7)cc6)c6cccc7oc8c9ccccc9c(-c9ccc%10ccccc%10c9)cc8c67)c54)c4ccccc34)ccc2c1] | 70.1% |
| 1-149 | 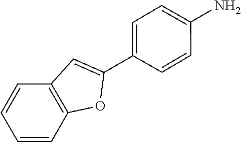 | 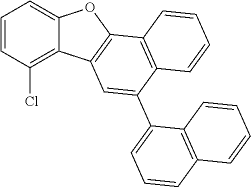 | 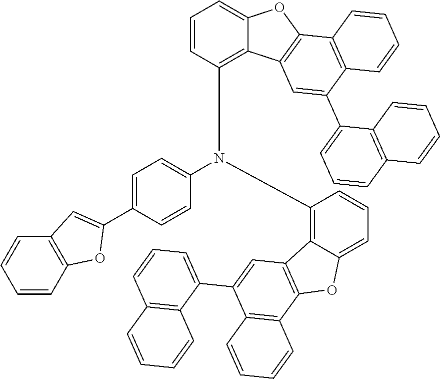 [SMILES: c1ccc2oc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2c1] | 72.6% |
| 1-154 | 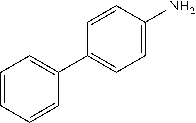 | 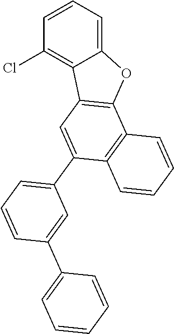 | 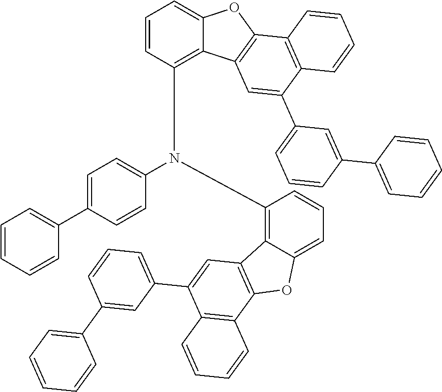 [SMILES: c1ccc(-c2ccc(N(c3cccc4oc5c6ccccc6c(-c6cccc(-c7ccccc7)c6)cc5c34)c3cccc4oc5c6ccccc6c(-c6cccc(-c7ccccc7)c6)cc5c34)cc2)cc1] | 84.8% |
| 1-179 | 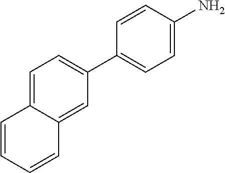 | 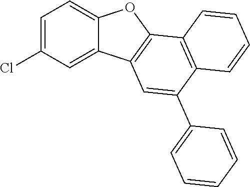 | 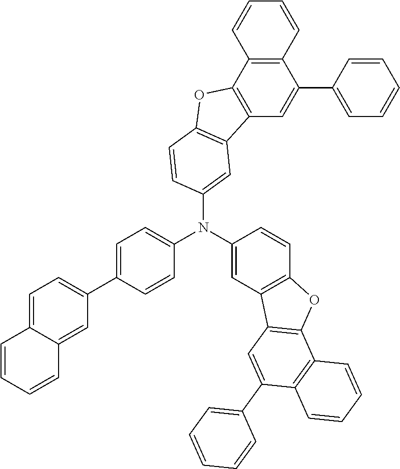 [SMILES: c1ccc(-c2cc3c4cc(N(c5ccc(-c6ccc7ccccc7c6)cc5)c5ccc6oc7c8ccccc8c(-c8ccccc8)cc7c6c5)ccc4oc3c3ccccc23)cc1] | 78.9% |
| 1-184 | 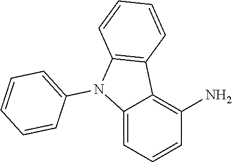 | | 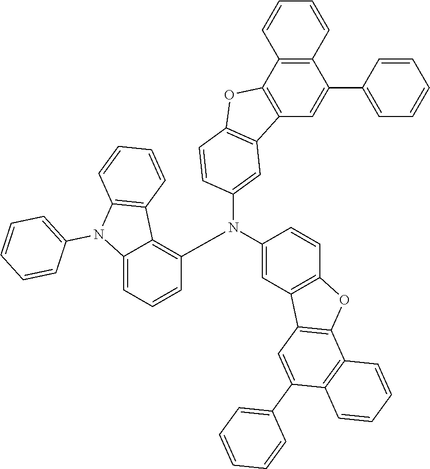 [SMILES: c1ccc(-c2cc3c4cc(N(c5ccc6oc7c8ccccc8c(-c8ccccc8)cc7c6c5)c5cccc6c5c5ccccc5n6-c5ccccc5)ccc4oc3c3ccccc23)cc1] | 76.5% |
| 1-221 | 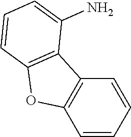 | 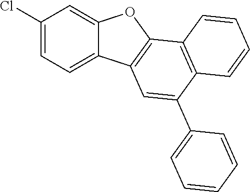 | 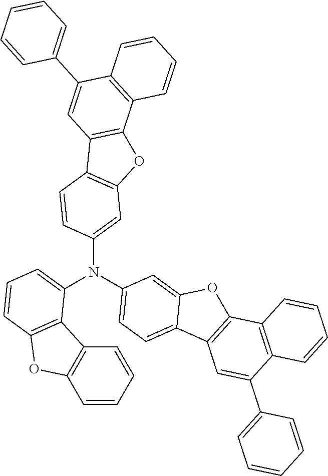 [SMILES: c1ccc(-c2cc3c4ccc(N(c5ccc6c(c5)oc5c7ccccc7c(-c7ccccc7)cc65)c5cccc6oc7ccccc7c56)cc4oc3c3ccccc23)cc1] | 80.1% |
| 1-218 | 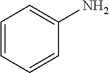 | 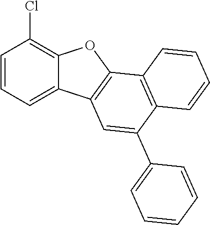 | 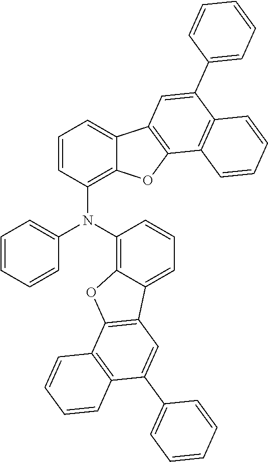 [SMILES: c1ccc(-c2cc3c4cccc(N(c5ccccc5)c5cccc6c5oc5c7ccccc7c(-c7ccccc7)cc65)c4oc3c3ccccc23)cc1] | 88.7% |
| 1-255 | 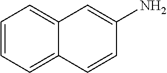 | 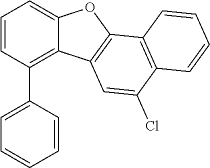 | 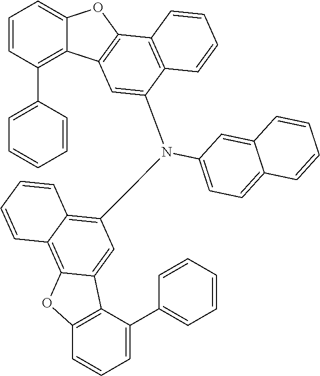 [SMILES: c1ccc(-c2cccc3oc4c5ccccc5c(N(c5ccc6ccccc6c5)c5cc6c(oc7cccc(-c8ccccc8)c76)c6ccccc56)cc4c23)cc1] | 86.2% |
| 1-284 | 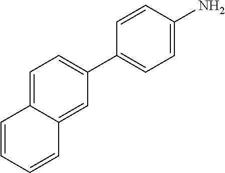 | 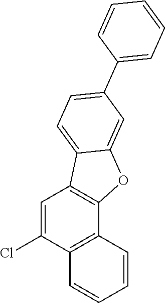 | 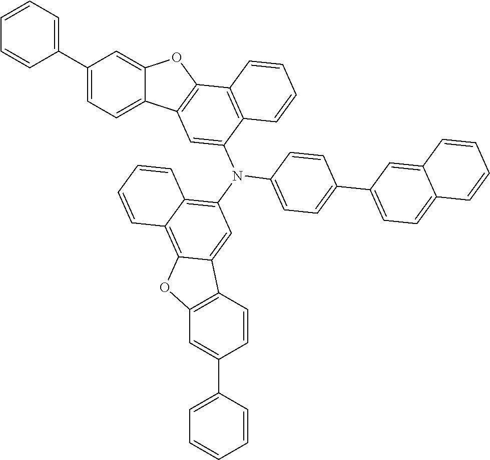 [SMILES: c1ccc(-c2ccc3c(c2)oc2c4ccccc4c(N(c4ccc(-c5ccc6ccccc6c5)cc4)c4cc5c6ccc(-c7ccccc7)cc6oc5c5ccccc45)cc32)cc1] | 74.8% |
| 1-302 | 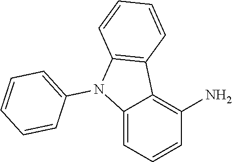 | 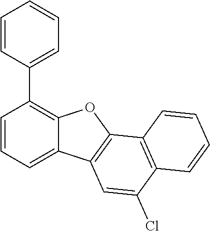 | 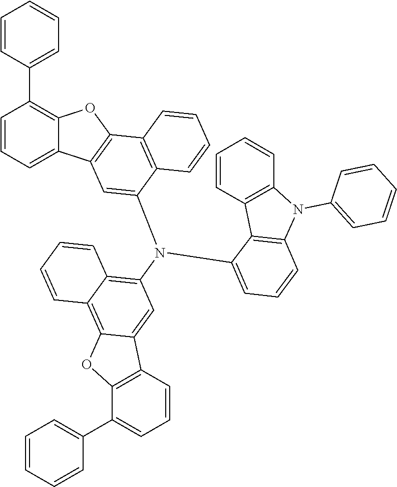 [SMILES: c1ccc(-c2cccc3c2oc2c4ccccc4c(N(c4cc5c6cccc(-c7ccccc7)c6oc5c5ccccc45)c4cccc5c4c4ccccc4n5-c4ccccc4)cc32)cc1] | 70.5% |
| 1-313 | 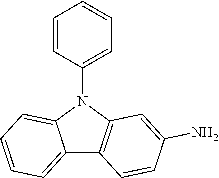 | 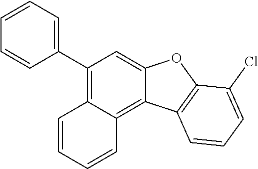 | 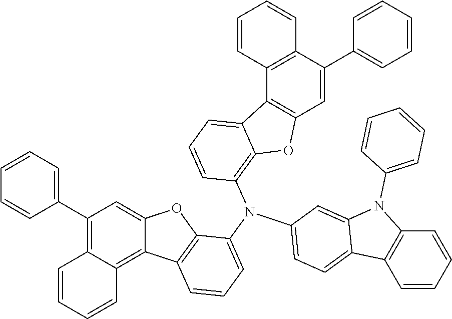 [SMILES: c1ccc(-c2cc3oc4c(N(c5ccc6c7ccccc7n(-c7ccccc7)c6c5)c5cccc6c5oc5cc(-c7ccccc7)c7ccccc7c56)cccc4c3c3ccccc23)cc1] | 73.6% |
| 1-321 | 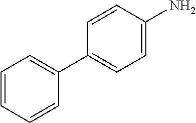 | 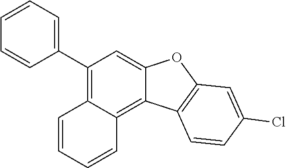 | 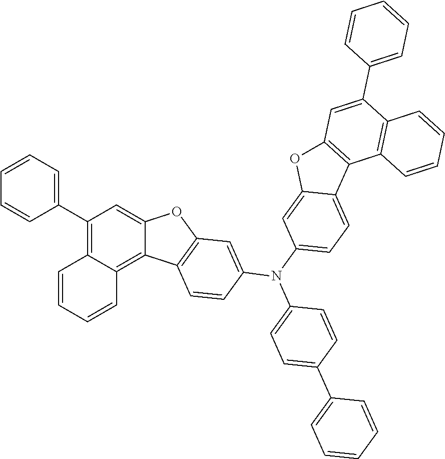 [SMILES: c1ccc(-c2ccc(N(c3ccc4c(c3)oc3cc(-c5ccccc5)c5ccccc5c34)c3ccc4c(c3)oc3cc(-c5ccccc5)c5ccccc5c34)cc2)cc1] | 86.4% |
| 1-340 | 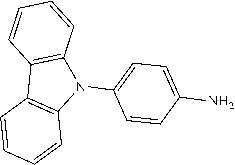 | 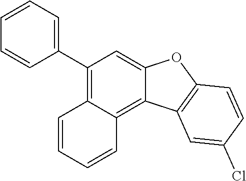 | 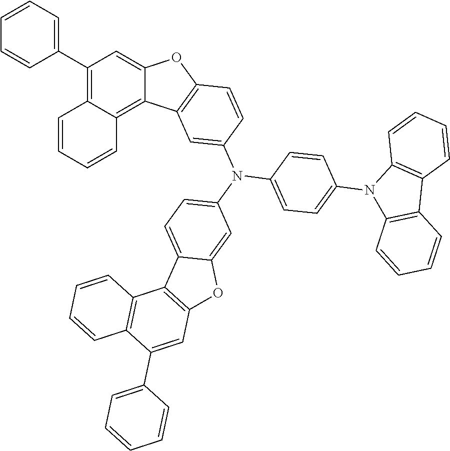 [SMILES: c1ccc(-c2cc3oc4cc(N(c5ccc(-n6c7ccccc7c7ccccc76)cc5)c5ccc6oc7cc(-c8ccccc8)c8ccccc8c7c6c5)ccc4c3c3ccccc23)cc1] | 77.6% |
| 1-350 | 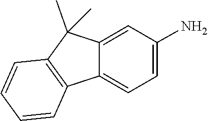 | 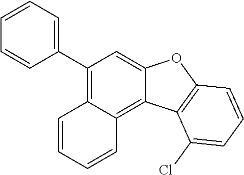 | 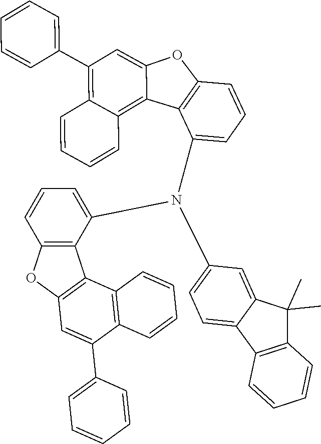 [SMILES: CC1(C)c2ccccc2-c2ccc(N(c3cccc4oc5cc(-c6ccccc6)c6ccccc6c5c34)c3cccc4oc5cc(-c6ccccc6)c6ccccc6c5c34)cc21] | 71.1% |
| 1-358 | 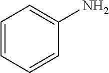 | 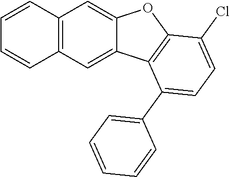 | 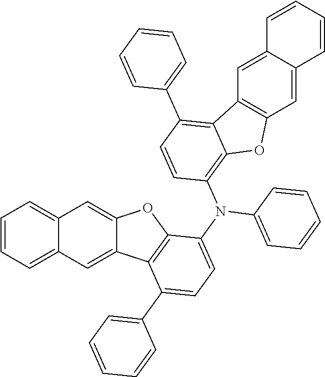 [SMILES: c1ccc(-c2ccc(N(c3ccccc3)c3ccc(-c4ccccc4)c4c3oc3cc5ccccc5cc34)c3oc4cc5ccccc5cc4c23)cc1] | 90.1% |
| 1-369 | 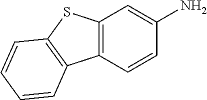 | | 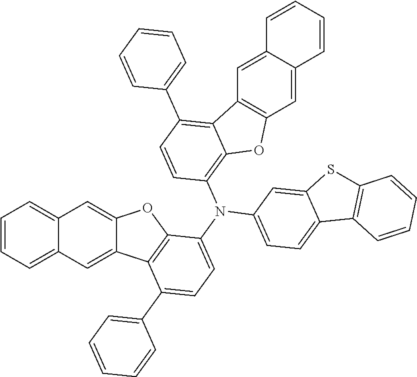 [SMILES: c1ccc(-c2ccc(N(c3ccc4c(c3)sc3ccccc34)c3ccc(-c4ccccc4)c4c3oc3cc5ccccc5cc34)c3oc4cc5ccccc5cc4c23)cc1] | 69.3% |
| 1-375 | 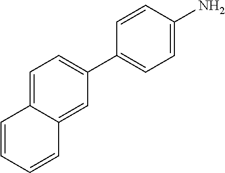 | 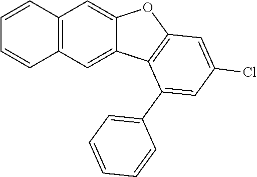 | 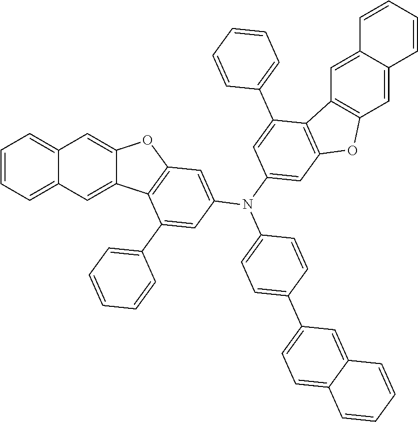 [SMILES: c1ccc(-c2cc(N(c3ccc(-c4ccc5ccccc5c4)cc3)c3cc(-c4ccccc4)c4c(c3)oc3cc5ccccc5cc34)cc3oc4cc5ccccc5cc4c23)cc1] | 89.7% |
| 1-413 | 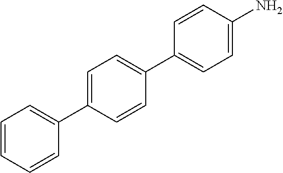 | 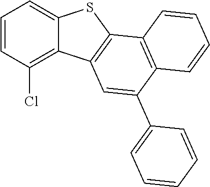 | 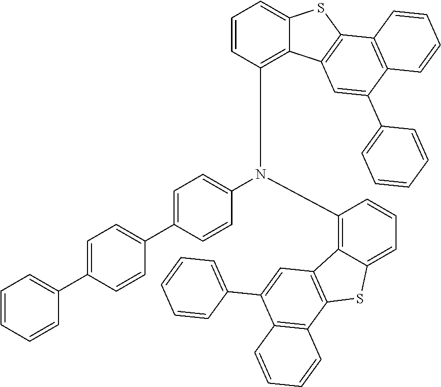 [SMILES: c1ccc(-c2ccc(-c3ccc(N(c4cccc5sc6c7ccccc7c(-c7ccccc7)cc6c45)c4cccc5sc6c7ccccc7c(-c7ccccc7)cc6c45)cc3)cc2)cc1] | 62.3% |
| 1-416 | 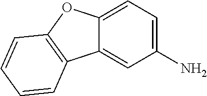 | 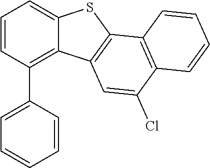 | 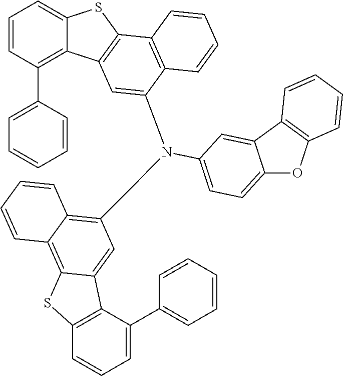 [SMILES: c1ccc(-c2cccc3sc4c5ccccc5c(N(c5ccc6oc7ccccc7c6c5)c5cc6c(sc7cccc(-c8ccccc8)c76)c6ccccc56)cc4c23)cc1] | 65.7% |
| 1-417 | 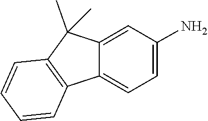 | 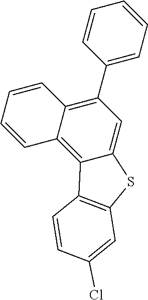 | 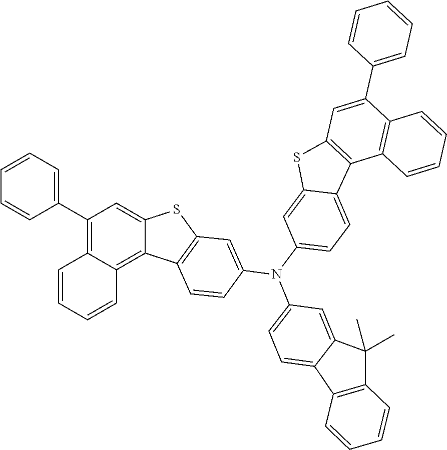 [SMILES: CC1(C)c2ccccc2-c2ccc(N(c3ccc4c(c3)sc3cc(-c5ccccc5)c5ccccc5c34)c3ccc4c(c3)sc3cc(-c5ccccc5)c5ccccc5c34)cc21] | 61.0% |
| 1-420 | 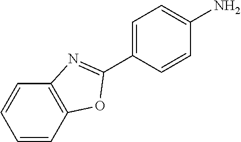 | 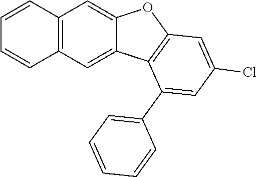 | 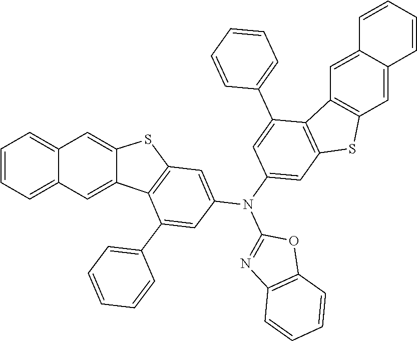 [SMILES: c1ccc(-c2cc(N(c3cc(-c4ccccc4)c4c(c3)sc3cc5ccccc5cc34)c3nc4ccccc4o3)cc3sc4cc5ccccc5cc4c23)cc1] | 63.5% |
| 1-422 | 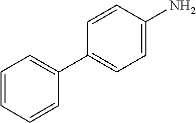 | 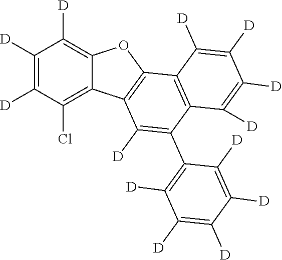 | 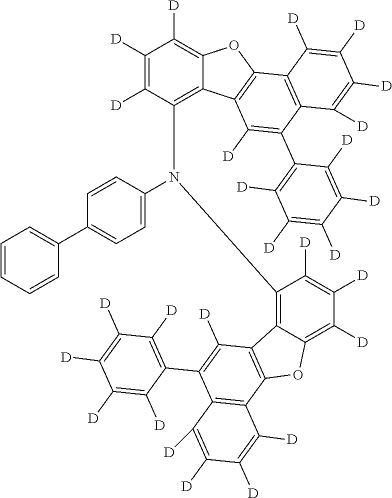 [SMILES: [2H]c1c([2H])c([2H])c(-c2c([2H])c3c(oc4c([2H])c([2H])c([2H])c(N(c5ccc(-c6ccccc6)cc5)c5c([2H])c([2H])c([2H])c6oc7c8c([2H])c([2H])c([2H])c([2H])c8c(-c8c([2H])c([2H])c([2H])c([2H])c8[2H])c([2H])c7c56)c43)c3c([2H])c([2H])c([2H])c([2H])c23)c([2H])c1[2H]] | 80.5% |
| 1-423 | 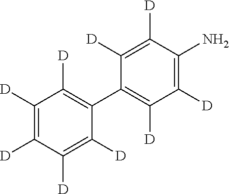 | 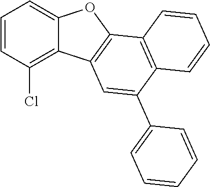 | 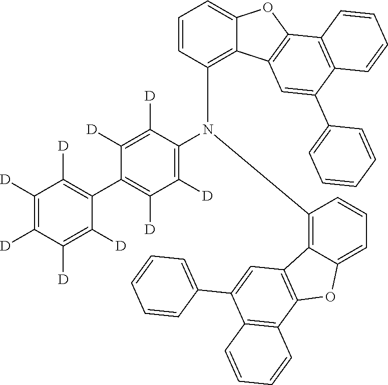 [SMILES: [2H]c1c([2H])c([2H])c(-c2c([2H])c([2H])c(N(c3cccc4oc5c6ccccc6c(-c6ccccc6)cc5c34)c3cccc4oc5c6ccccc6c(-c6ccccc6)cc5c34)c([2H])c2[2H])c([2H])c1[2H]] | 84.7% |

### Preparative Example 2: Preparation of Compound 1-426

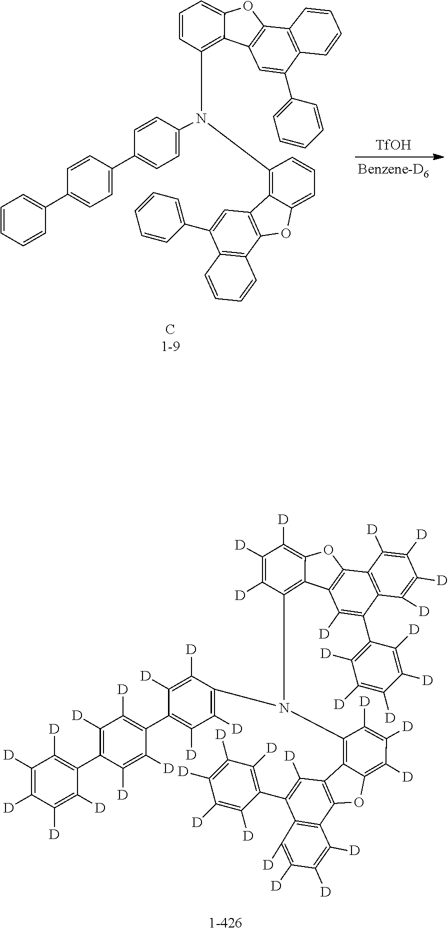

Compound 1-12 (C, 10 g, 12.05 mmol) was introduced into a 1000 mL round-bottom flask and dissolved in benzene-D₆ (100 mL). The reaction mixture was placed in an ice bath. Trifluoromethanesulfonic acid (7.58 mL, 84.34 mmol) was slowly added dropwise, and the mixture was stirred at 60° C. for 4 hours. After completion of the reaction, the reaction temperature was lowered to room temperature and the flask was placed in an ice bath. After neutralization with an aqueous K₃PO4 solution, the mixture was extracted with DCM. The organic layer was dried over Mg₂SO₄ and concentrated under reduced pressure to remove the solvent. The residue was purified by silica gel column chromatography and recrystallized to obtain Compound 1-426 (8 g, 9.2 mmol, 90.3%) as a solid.

The target compound was prepared in the same manner as in Preparative Example 2, except that Compound C shown in Table 2 was used instead of 1-12, as shown in Table 2.

TABLE 2

| Com- | | | |
|---|---|---|---|
| pound | | | |
| No. | C | Target compound | Yield |
| 1-426 | 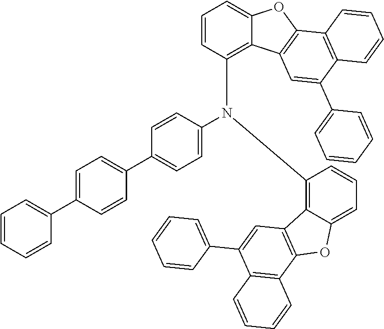 [SMILES: c1ccc(-c2ccc(-c3ccc(N(c4cccc5oc6c7ccccc7c(-c7ccccc7)cc6c45)c4cccc5oc6c7ccccc7c(-c7ccccc7)cc6c45)cc3)cc2)cc1] | 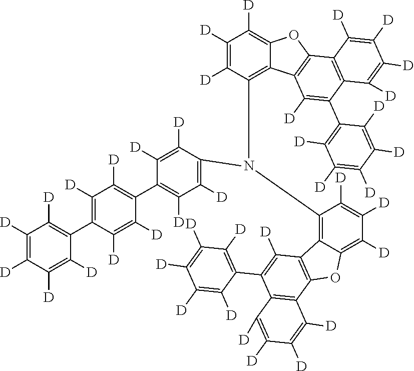 [SMILES: [2H]c1c([2H])c([2H])c(-c2c([2H])c([2H])c(-c3c([2H])c([2H])c(N(c4c([2H])c([2H])c([2H])c5oc6c7c([2H])c([2H])c([2H])c([2H])c7c(-c7c([2H])c([2H])c([2H])c([2H])c7[2H])c([2H])c6c45)c4c([2H])c([2H])c([2H])c5oc6c7c([2H])c([2H])c([2H])c([2H])c7c(-c7c([2H])c([2H])c([2H])c([2H])c7[2H])c([2H])c6c45)c([2H])c3[2H])c([2H])c2[2H])c([2H])c1[2H]] | 90.3% |
| 1-432 | 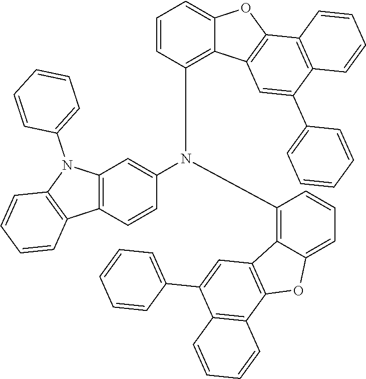 [SMILES: c1ccc(-c2cc3c(oc4cccc(N(c5ccc6c7ccccc7n(-c7ccccc7)c6c5)c5cccc6oc7c8ccccc8c(-c8ccccc8)cc7c56)c43)c3ccccc23)cc1] | 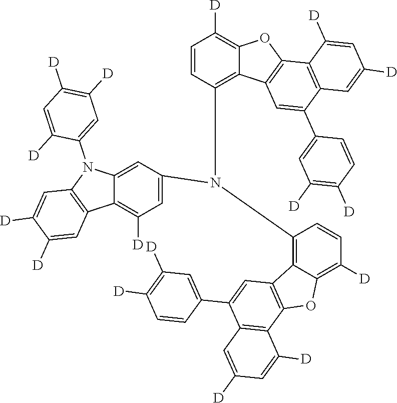 [SMILES: [2H]c1cc([2H])c2c(c1)c(-c1ccc([2H])c([2H])c1)cc1c2oc2c([2H])ccc(N(c3cc([2H])c4c5cc([2H])c([2H])cc5n(-c5cc([2H])c([2H])cc5[2H])c4c3)c3ccc([2H])c4oc5c(cc(-c6ccc([2H])c([2H])c6)c6cc([2H])cc([2H])c65)c34)c21] | 88.2% |
| 1-439 | 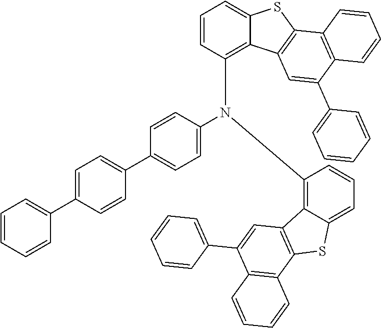 [SMILES: c1ccc(-c2ccc(-c3ccc(N(c4cccc5sc6c7ccccc7c(-c7ccccc7)cc6c45)c4cccc5sc6c7ccccc7c(-c7ccccc7)cc6c45)cc3)cc2)cc1] | 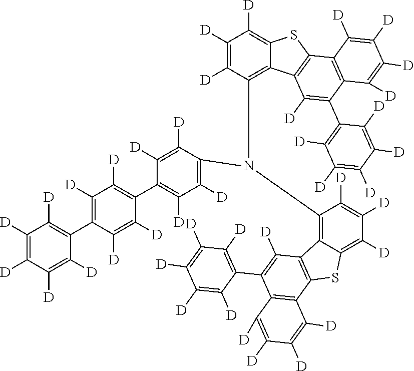 [SMILES: [2H]c1c([2H])c([2H])c(-c2c([2H])c([2H])c(-c3c([2H])c([2H])c(N(c4c([2H])c([2H])c([2H])c5sc6c7c([2H])c([2H])c([2H])c([2H])c7c(-c7c([2H])c([2H])c([2H])c([2H])c7[2H])c([2H])c6c45)c4c([2H])c([2H])c([2H])c5sc6c7c([2H])c([2H])c([2H])c([2H])c7c(-c7c([2H])c([2H])c([2H])c([2H])c7[2H])c([2H])c6c45)c([2H])c3[2H])c([2H])c2[2H])c([2H])c1[2H]] | 84.5% |

### Preparative Example 3: Preparation of Compound 2-1

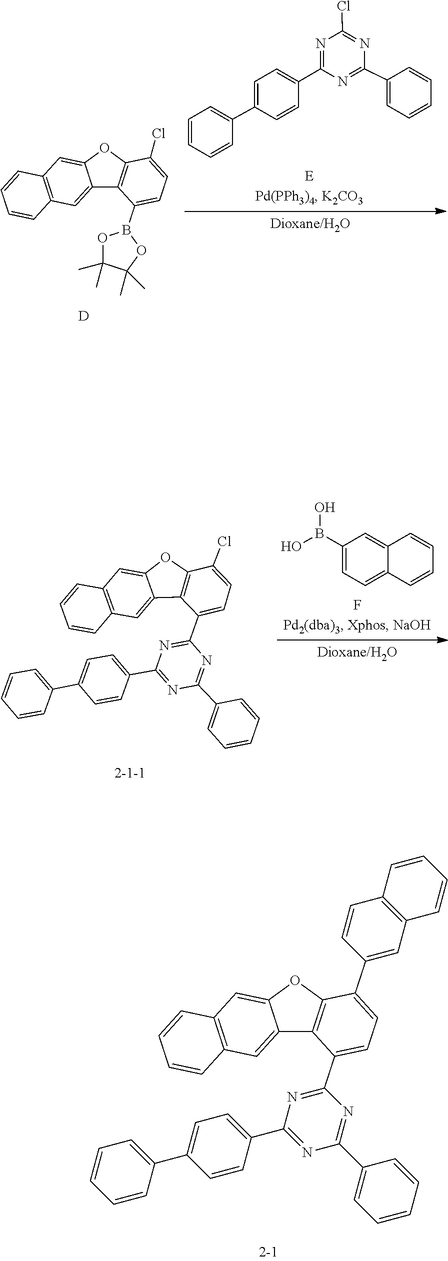

### 1) Preparation of Intermediate 2-1-1

2-(4-chloronaphtho[2,3-b]benzofuran-1-yl)-4,4,5,5-tetramethyl-1,3,2-dioxaborolane (D, 10 g, 26.41 mmol), 2-([1,1′-biphenyl]-4-yl)-4-chloro-6-phenyl-1,3,5-triazine (E, 8.9 g 25.89 mmol), Pd(PPh₃)₄ (1.14 g 0.99 mmol), and K₂CO₃ (5.6 g 52.82 mmol) were introduced into a 500 mL round-bottom flask, followed by the addition of 1,4-dioxane (100 mL)/H₂O (20 mL), and the reaction mixture was refluxed at 110° C. for 3 hours. After completion of the reaction, the reaction temperature was lowered to room temperature, and the precipitated solid was filtered. The filtered solid was purified by silica gel column chromatography and recrystallized to obtain Intermediate 2-1-1 (12.5 g 22.32 mmol, 84.52%) as a solid compound.

### 2) Preparation of Compound 2-1

Intermediate 2-1-1 (12.5 g 22.32 mmol), naphthalen-2-ylboronic acid (F, 4.61 g 26.78 mmol), Pd₂(dba)₃ (0.82 g, 0.89 mmol), Xphos (0.85 g, 1.79 mmol), and NaOH (1.79 g, 44.64 mmol) were introduced into a 500 mL round-bottom flask, followed by the addition of 1,4-dioxane (125 mL)/H₂O (25 mL), and the reaction mixture was refluxed at 120° C. for 3 hours. After completion of the reaction, the reaction temperature was lowered to room temperature, and the precipitated solid was filtered. The filtered solid was purified by silica gel column chromatography and recrystallized to obtain compound 2-1 (11.7 g 17.95 mmol, 80.43%) as a solid.

The target compound was prepared in the same manner as in Preparative Example 3, except that Compound D shown in Table 3 was used instead of 2-(4-chloronaphtho[2,3-b]benzofuran-1-yl)-4,4,5,5-tetramethyl-1,3,2-dioxaborolane, Compound E shown in Table 3 was used instead of 2-([1,1′-biphenyl]-4-yl)-4-chloro-6-phenyl-1,3,5-triazine, and Compound F shown in Table 3 was used instead of naphthalen-2-ylboronic acid, as shown in Table 3 below.

TABLE 3

| Compound | | | |
|---|---|---|---|
| No. | D | E | F |
| 2-5 | 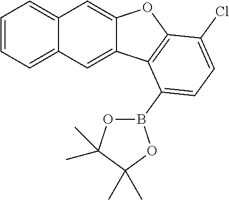 | 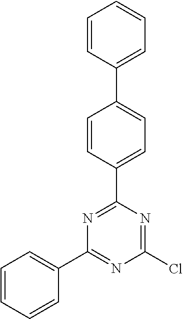 | 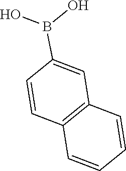 |
| 2-27 | 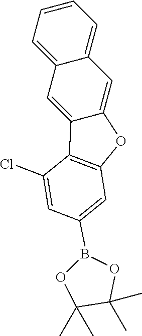 | 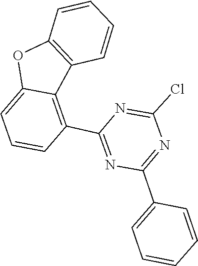 | 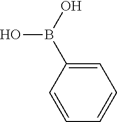 |
| 2-35 | 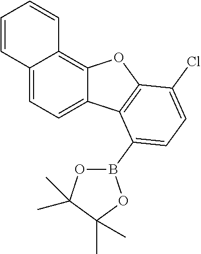 | 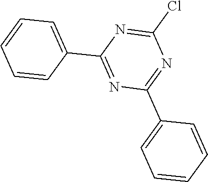 | 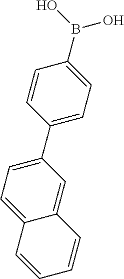 |
| 2-52 | 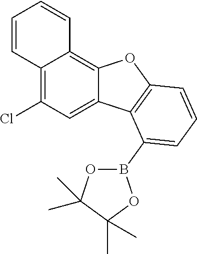 | 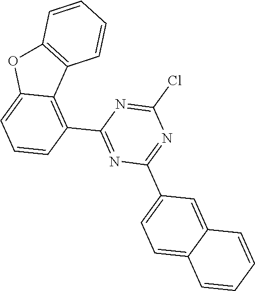 | 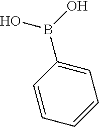 |
| 2-84 | 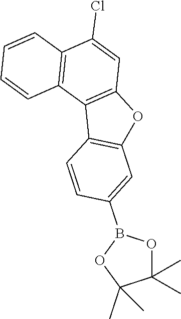 | 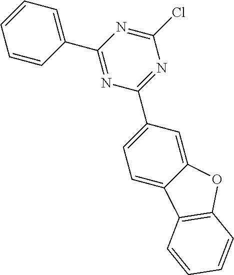 | 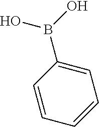 |
| 2-114 | 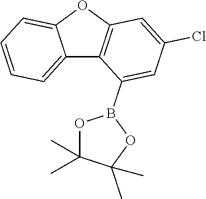 | 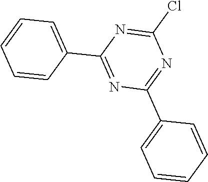 | 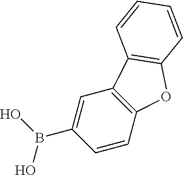 |
| 2-131 | 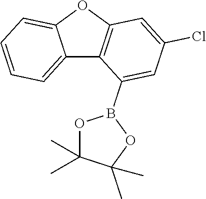 | 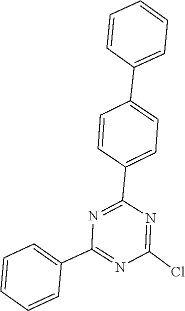 | 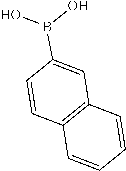 |
| 2-169 | 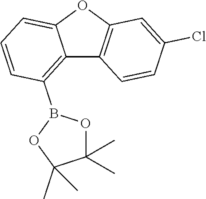 | 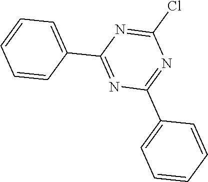 | 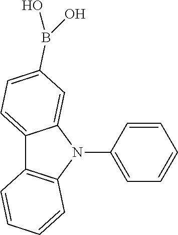 |

| Compound | | |
|---|---|---|
| No. | Target compound | Yield |
| 2-5 | 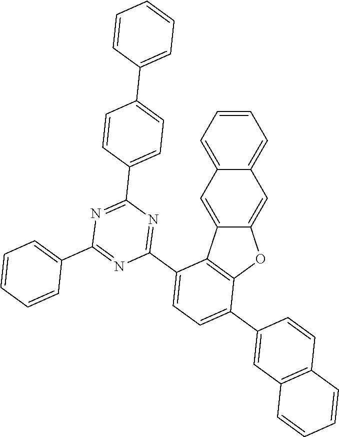 [SMILES: c1ccc(-c2ccc(-c3nc(-c4ccccc4)nc(-c4ccc(-c5ccc6ccccc6c5)c5oc6cc7ccccc7cc6c45)n3)cc2)cc1] | 78.4% |
| 2-27 | 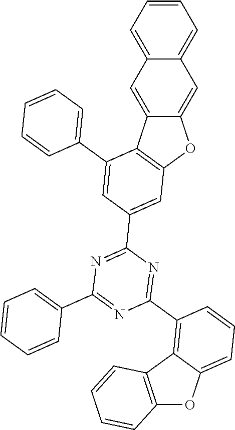 [SMILES: c1ccc(-c2nc(-c3cc(-c4ccccc4)c4c(c3)oc3cc5ccccc5cc34)nc(-c3cccc4oc5ccccc5c34)n2)cc1] | 73.7% |
| 2-35 | 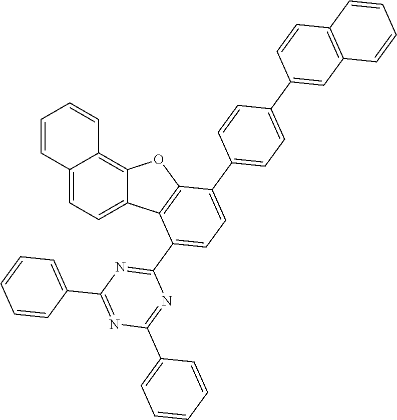 [SMILES: c1ccc(-c2nc(-c3ccccc3)nc(-c3ccc(-c4ccc(-c5ccc6ccccc6c5)cc4)c4oc5c6ccccc6ccc5c34)n2)cc1] | 70.0% |
| 2-52 | 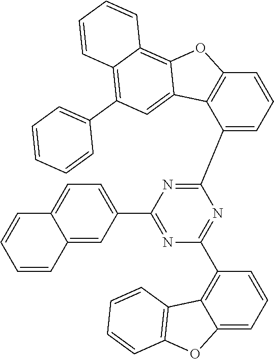 [SMILES: c1ccc(-c2cc3c(oc4cccc(-c5nc(-c6ccc7ccccc7c6)nc(-c6cccc7oc8ccccc8c67)n5)c43)c3ccccc23)cc1] | 75.8% |
| 2-84 | 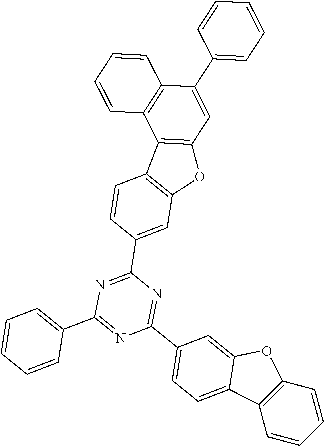 [SMILES: c1ccc(-c2nc(-c3ccc4c(c3)oc3ccccc34)nc(-c3ccc4c(c3)oc3cc(-c5ccccc5)c5ccccc5c34)n2)cc1] | 60.8% |
| 2-114 | 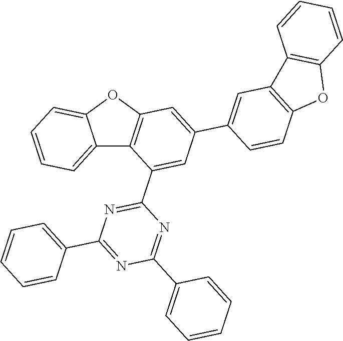 [SMILES: c1ccc(-c2nc(-c3ccccc3)nc(-c3cc(-c4ccc5oc6ccccc6c5c4)cc4oc5ccccc5c34)n2)cc1] | 85.6% |
| 2-131 | 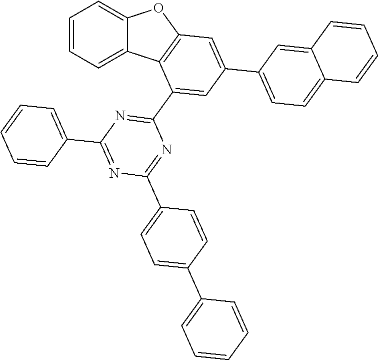 [SMILES: c1ccc(-c2ccc(-c3nc(-c4ccccc4)nc(-c4cc(-c5ccc6ccccc6c5)cc5oc6ccccc6c45)n3)cc2)cc1] | 83.9% |
| 2-169 | 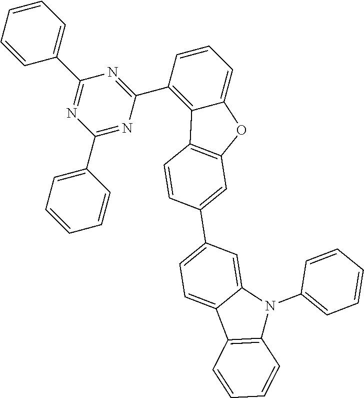 [SMILES: c1ccc(-c2nc(-c3ccccc3)nc(-c3cccc4oc5cc(-c6ccc7c8ccccc8n(-c8ccccc8)c7c6)ccc5c34)n2)cc1] | 70.3% |

The ¹H NMR and FD-MS analysis results of the above compounds are shown in Tables 4 and 5, thereby confirming the synthesis of the target compounds. Among the compounds, 1-426 and 1-439 had a deuterium content of 100%, and thus ¹H NMR analysis was not possible.

TABLE 4

| Compound | ¹H NMR (CDCl₃, 400 MHz) |
|---|---|
| 1-9 | δ = 8.97(2H, d), 8.18(2H, d), 7.79(4H, d), 7.75(2H, d), 7.64(2H, d), 7.55(2H, t), 7.49(2H, |
| | dd), 7.48(2H, t), 7.46(4H, t), 7.41(3H, t), 7.38(2H, td), 7.27(2H, s), 7.24(2H, td), 7.16(2H, |
| | t), 6.91(2H, d) |
| 1-12 | δ = 8.97(2H, d), 8.18(2H, d), 7.79(4H, d), 7.75(2H, d), 7.64(2H, d), 7.55(2H, t), 7.49(2H, |
| | dd), 7.48(2H, t), 7.46(4H, t), 7.41(3H, t), 7.38(2H, s), 7.27(2H, d), 7.25(4H, td), 7.24(2H, |
| | t), 7.16(2H, d), 6.91 (2H, d) |
| 1-20 | δ = 8.97(2H, d), 8.18(2H, d), 7.9(1H, d), 7.86(1H, d), 7.79(4H, d), 7.64(2H, t), 7.55(1H, |
| | dd), 7.48(2H, t), 7.46(4H, t), 7.41(2H, t), 7.38(3H, td), 7.33(1H, s), 7.28(1H, td), 7.24(2H, |
| | t), 7.16(3H, d), 6.91 (2H, d), 1.69 (6H, s) |
| 1-29 | δ = 8.97(2H, d), 8.55(1H, d), 8.24(1H, d), 8.18(2H, d), 7.79(4H, d), 7.64(2H, t), 7.62(2H, |
| | dd), 7.58(1H, t), 7.52(1H, t), 7.5(2H, t), 7.48(2H, d), 7.46(4H, d), 7.41(2H, td), 7.38(2H, |
| | s), 7.27(1H, d), 7.25 (1H, d), 7.24 (2H, d), 7.16 (3H, dd), 7.11 (1H, dd), 6.91 (2H, d) |
| 1-45 | δ = 8.97(2H, d), 8.45(1H, d), 8.18(2H, d), 7.95(1H, d), 7.86(1H, d), 7.85(1H, t), 7.79(4H, |
| | dd), 7.64(2H, t), 7.56(1H, t), 7.48(2H, t), 7.46(4H, d), 7.41(3H, d), 7.38(2H, td), 7.31(1H, |
| | s), 7.24(2H, d), 7.16 (2H, d), 6.91 (2H, d) |
| 1-48 | δ = 8.97(2H, d), 8.18(2H, d), 8.03(1H, d), 7.98(1H, d), 7.79(4H, d), 7.64(2H, t), 7.55(1H, |
| | dd), 7.54(1H, t), 7.48(2H, t), 7.46(4H, t), 7.41(2H, d), 7.39(1H, d), 7.38(2H, td), 7.31(1H, |
| | s), 7.24(2H, d), 7.16 (2H, d), 6.91 (3H, d) |
| 1-117 | δ = 9.24(1H, s), 8.97(2H, d), 8.7(1H, d), 8.33(1H, d), 8.18(2H, d), 8.09(2H, t), 8.06(2H, |
| | dd), 7.99(2H, t), 7.64(2H, t), 7.63(2H, t), 7.58(2H, d), 7.55(4H, d), 7.48(2H, td), 7.47(1H, |
| | d), 7.38(4H, s), 7.27 (2H, d), 7.24 (2H, d), 7.16 (2H, dd), 6.91 (2H, dd) |
| 1-123 | δ = 8.97(2H, d), 8.18(3H, d), 8.09(2H, d), 8.06(2H, d), 8.02(1H, d), 7.99(2H, t), 7.85(2H, |
| | dd), 7.64(2H, t), 7.63(2H, t), 7.58(2H, t), 7.55(2H, d), 7.53(1H, d), 7.51(1H, td), 7.48(2H, |
| | d), 7.38(4H, s), 7.27 (2H, d), 7.24 (2H, d), 7.16 (2H, dd), 6.91 (2H, dd) |
| 1-149 | δ = 8.97(2H, d), 8.95(2H, d), 8.5(2H, d), 8.18(2H, d), 7.89(2H, d), 7.78(2H, t), 7.77(2H, |
| | dd), 7.64(2H, t), 7.63(2H, t), 7.59(1H, t), 7.5(1H, d), 7.48(2H, d), 7.41(2H, td), 7.38(2H, |
| | s), 7.35(2H, d), 7.28 (1H, d), 7.27 (2H, d), 7.24 (2H, dd), 7.22 (1H, dd), 7.16 (2H, d), 6.91 |
| | (2H, d), 6.76 (1H, s) |
| 1-154 | δ = 8.97(2H, d), 8.18(2H, d), 7.94(2H, d), 7.75(6H, d), 7.71(2H, d), 7.64(2H, t), 7.61(4H, |
| | dd), 7.55(2H, t), 7.49(6H, t), 7.48(2H, t), 7.41(3H, d), 7.38(2H, s), 7.27(2H, td), 7.24(2H, |
| | d), 7.16(2H, d), 6.91 (2H, d) |
| 1-179 | δ = 8.97(2H, d), 8.22(2H, d), 8.18(2H, d), 8.09(1H, d), 8.06(1H, d), 7.99(1H, t), 7.79(4H, |
| | dd), 7.64(2H, t), 7.63(1H, t), 7.58(1H, t), 7.55(3H, d), 7.48(2H, d), 7.46(6H, td), 7.41(2H, |
| | d), 7.38(3H, s), 7.27 (2H, d), 6.97 (2H, d) |
| 1-184 | δ = 8.97(2H, d), 8.22(2H, d), 8.19(1H, d), 8.18(2H, d), 7.79(4H, d), 7.64(2H, t), 7.62(2H, |
| | dd), 7.58(1H, t), 7.5(2H, t), 7.48(2H, t), 7.46(6H, d), 7.41(2H, d), 7.4(1H, td), 7.38(2H, s), |
| | 7.2(1H, d), 7.18 (2H, d), 7.01 (1H, d), 6.97 (2H, dd), 6.4 (1H, dd) |
| 1-221 | δ = 8.97(2H, d), 8.18(2H, d), 8.03(2H, d), 7.98(1H, d), 7.79(4H, d), 7.64(2H, t), 7.55(2H, |
| | dd), 7.54(1H, t), 7.48(2H, t), 7.46(4H, t), 7.41(2H, d), 7.39(1H, d), 7.38(2H, s), 7.31(1H, |
| | d), 7.24(1H, d), 7.16 (1H, d), 6.91 (3H, d) |
| 1-218 | δ = 8.97(2H, d), 8.18(2H, d), 7.79(4H, d), 7.64(4H, d), 7.48(2H, d), 7.46(4H, t), 7.41(2H, |
| | dd), 7.38(2H, t), 7.24(2H, t), 7.18(2H, t), 7.08(2H, d), 7(1H, d), 6.97(2H, s) |
| 1-255 | δ = 8.22(2H, s), 8.18(2H, d), 8.12(2H, d), 7.79(4H, d), 7.71(1H, d), 7.64(2H, t), 7.62(1H, |
| | dd), 7.6(2H, t), 7.57(2H, t), 7.54(1H, t), 7.5(2H, t), 7.47(2H, t), 7.46(4H, t), 7.45(1H, d), |
| | 7.42(1H, d), 7.41 (2H, d), 7.38 (1H, d), 7.11 (1H, dd) |
| 1-284 | δ = 8.22(2H, s), 8.18(2H, d), 8.12(2H, d), 8.09(1H, d), 8.06(1H, d), 7.99(1H, t), 7.78(2H, |
| | dd), 7.76(2H, t), 7.75(4H, t), 7.64(2H, t), 7.63(1H, t), 7.58(1H, t), 7.57(2H, t), 7.55(3H, d), |
| | 7.5(2H, d), 7.49 (4H, d), 7.41 (2H, d), 7.38 (1H, dd), 7.27 (2H, dd) |
| 1-302 | δ = 8.55(1H, d), 8.22(2H, s), 8.18(2H, d), 8.12(2H, d), 8.08(2H, d), 8.02(2H, t), 7.64(2H, |
| | dd), 7.62(2H, t), 7.58(1H, t), 7.52(1H, t), 7.51(4H, t), 7.5(4H, t), 7.46(4H, t), 7.41(4H, d), |
| | 7.27(1H, d), 7.16 (1H, d), 7.14 (1H, d), 7.11 (1H, dd), 7.06 (1H, dd) |
| 1-313 | δ = 8.97(4H, d), 8.19(1H, d), 7.96(1H, d), 7.79(4H, d), 7.64(2H, d), 7.62(2H, t), 7.58(1H, |
| | dd), 7.5(2H, t), 7.48(4H, t), 7.46(4H, dd), 7.41(2H, d), 7.4(1H, t), 7.31(2H, s), 7.2(1H, d), |
| | 7.18(4H, d), 6.97 (2H, d), 6.4 (1H, d) |
| 1-321 | δ = 8.97(4H, d), 8.03(2H, s), 7.79(4H, d), 7.75(2H, d), 7.55(4H, d), 7.49(2H, t), 7.48(4H, |
| | dd), 7.46(4H, t), 7.41(3H, t), 7.31(2H, s), 7.27(2H, d), 6.91(2H, d) |
| 1-340 | δ = 8.97(4H, d), 8.55(1H, d), 8.22(1H, s), 8.19(1H, d), 8.03(1H, s), 7.79(4H, t), 7.69(2H, |
| | dd), 7.55(1H, t), 7.52(1H, t), 7.48(4H, s), 7.46(5H, d), 7.41(2H, d), 7.4(1H, d), 7.31(2H, |
| | s), 7.22(2H, d), 7.2 (1H, d), 7.18 (1H, d), 7.16 (1H, dd), 7.11 (1H, dd), 6.97 (1H, d), 6.91 |
| | (1H, d) |
| 1-350 | δ = 8.97(4H, d), 7.9(1H, d), 7.86(1H, d), 7.79(4H, d), 7.55(1H, s), 7.48(4H, t), 7.46(4H, |
| | dd), 7.41(2H, t), 7.38(1H, t), 7.33(1H, d), 7.31(2H, s), 7.28(1H, d), 7.24(2H, t), 7.16(3H, |
| | d), 6.91(2H, d), 1.69 (6H, s) |
| 1-358 | δ = 8.28(2H, d), 7.84(2H, d), 7.79(4H, d), 7.75(2H, d), 7.53(2H, d), 7.49(2H, t), 7.48(2H, |
| | dd), 7.46(4H, t), 7.42(2H, t), 7.41(2H, d), 7.24(2H, s), 7.16(2H, d), 7.08(2H, t), 7(1H, t) |
| 1-369 | δ = 8.45(1H, d), 8.28(2H, d), 7.86(1H, d), 7.84(2H, d), 7.79(4H, d), 7.75(2H, t), 7.74(1H, |
| | dd), 7.64(1H, s), 7.56(1H, t), 7.53(2H, d), 7.49(2H, s), 7.48(2H, d), 7.46(4H, t), 7.43(1H, |
| | t), 7.42(2H, d), 7.41 (2H, d), 7.31 (1H, t), 7.16 (2H, d) |
| 1-375 | δ = 8.28(2H, d), 8.2(2H, s), 8.13(2H, d), 8.09(1H, d), 8.06(1H, d), 7.99(1H, t), 7.84(2H, |
| | dd), 7.79(4H, s), 7.75(2H, t), 7.63(1H, d), 7.58(1H, s), 7.55(3H, d), 7.49(2H, t), 7.48(2H, |
| | t), 7.46(4H, d), 7.42 (2H, d), 7.41 (2H, t), 7.38 (1H, d), 7.27 (2H, d) |
| 1-413 | δ = 8.97(2H, d), 7.84(2H, d), 7.79(4H, d), 7.75(2H, d), 7.67(2H, s), 7.55(2H, t), 7.52(2H, |
| | dd), 7.49(2H, s), 7.48(4H, t), 7.46(4H, d), 7.43(2H, s), 7.41(3H, d), 7.27(4H, t), 7.25(4H, |
| | t) |
| 1-416 | δ = 8.22(1H, d), 8.12(2H, d), 7.98(1H, d), 7.96(2H, d), 7.95(2H, s), 7.84(2H, t), 7.79(4H, |
| | dd), 7.67(2H, d), 7.54(1H, t), 7.5(4H, t), 7.48(2H, t), 7.46(5H, d), 7.41(2H, t), 7.39(1H, t), |
| | 7.31(1H, t), 6.97 (1H, t) |
| 1-417 | δ = 8.97(4H, d), 7.9(1H, d), 7.86(1H, d), 7.79(4H, d), 7.75(2H, s), 7.74(2H, t), 7.64(2H, |
| | dd), 7.55(1H, d), 7.48(4H, t), 7.46(4H, t), 7.43(2H, t), 7.41(2H, d), 7.38(1H, t), 7.33(1H, |
| | t), 7.28(1H, t), 7.16 (1H, t), 1.69 (6H, d) |
| 1-420 | δ = 8.28(2H, d), 7.86(2H, d), 7.84(4H, d), 7.79(4H, d), 7.78(2H, s), 7.75(2H, t), 7.74(2H, |
| | dd), 7.72(2H, d), 7.48(2H, t), 7.46(4H, t), 7.41(2H, t), 7.39(2H, d) |
| 1-422 | δ = 7.75(2H, d), 7.55(2H, d), 7.49(2H, d), 7.41(1H, t), 7.27(2H, d) |
| 1-423 | δ = 8.97(2H, d), 8.18(2H, d), 7.79(4H, d), 7.64(2H, t), 7.48(2H, d), 7.46(4H, t), 7.41(2H, |
| | dd), 7.38(2H, s), 7.24(2H, t), 7.16(2H, d), 6.91(2H, d) |
| 1-432 | δ = 8.06(2H, d), 7.84(2H, d), 7.67(2H, d), 7.63(1H, t), 7.52(2H, d), 7.48(3H, t), 7.47(1H, |
| | dd), 7.45(1H, s), 7.38(2H, t), 7.26(1H, d), 7.24(2H, d), 7(2H, d), 6.86(1H, t) |
| 2-5 | δ = 8.36(2H, d), 8.28(1H, d), 8.25(2H, d), 8.09(1H, t), 8.06(1H, d), 7.99(1H, t), 7.84(1H, |
| | dd), 7.82(1H, s), 7.76(1H, t), 7.75(3H, d), 7.63(1H, d), 7.58(1H, d), 7.55(1H, t), 7.5(3H, |
| | t), 7.49(3H, d), 7.48 (1H, d), 7.42 (1H, t), 7.41 (1H, d), 7.38 (1H, d), 7.25 (2H, d) |
| 2-27 | δ = 8.36(2H, d), 8.28(1H, d), 7.98(1H, d), 7.86(1H, s), 7.84(1H, d), 7.81(1H, s), 7.79(2H, |
| | dd), 7.75(1H, d), 7.6(1H, t), 7.57(1H, d), 7.54(1H, d), 7.5(3H, d), 7.49(1H, t), 7.48(1H, t), |
| | 7.47(1H, d), 7.46 (2H, d), 7.42 (1H, t), 7.41 (1H, d), 7.39 (1H, d), 7.31 (1H, t) |
| 2-35 | δ = 8.36(4H, d), 8.19(1H, d), 8.09(1H, d), 8.06(1H, d), 7.99(1H, d), 7.84(1H, t), 7.82(1H, |
| | dd), 7.76(1H, d), 7.63(1H, t), 7.59(1H, d), 7.58(1H, d), 7.55(1H, d), 7.5(6H, t), 7.48(2H, |
| | t), 7.38(1H, d), 7.25 (4H, d), 7.23 (1H, d) |
| 2-52 | δ = 9.09(1H, s), 8.97(1H, d), 8.49(1H, d), 8.18(1H, d), 8.16(1H, d), 8.08(1H, t), 8(1H, dd), |
| | 7.98(1H, d), 7.79(2H, t), 7.64(1H, d), 7.61(1H, d), 7.6(2H, d), 7.57(3H, t), 7.54(1H, t), |
| | 7.48(1H, d), 7.47 (2H, d), 7.46 (2H, d), 7.41 (1H, d), 7.39 (1H, d), 7.38 (1H, s), 7.31 (1H, |
| | d) |
| 2-84 | δ = 8.97(2H, d), 8.36(2H, d), 7.98(1H, d), 7.79(2H, d), 7.78(2H, d), 7.76(2H, t), 7.57(2H, |
| | dd), 7.54(1H, d), 7.5(3H, t), 7.48(2H, d), 7.46(2H, d), 7.41(1H, d), 7.39(1H, t), 7.31(2H, |
| | t) |
| 2-114 | δ = 8.36(4H, d), 7.98(2H, d), 7.86(1H, d), 7.83(1H, d), 7.81(1H, s), 7.69(1H, t), 7.63(1H, |
| | dd), 7.54(2H, d), 7.5(6H, t), 7.39(2H, t), 7.31(2H, t) |
| 2-131 | δ = 8.36(2H, d), 8.25(2H, d), 8.09(1H, d), 8.06(1H, d), 7.99(1H, t), 7.98(1H, t), 7.86(1H, |
| | dd), 7.81(1H, s), 7.75(2H, t), 7.63(1H, t), 7.58(1H, t), 7.55(1H, d), 7.54(1H, t), 7.5(3H, t), |
| | 7.49(2H, d), 7.41 (1H, d), 7.39 (1H, d), 7.38 (1H, d), 7.31 (1H, d), 7.25 (2H, d) |
| 2-169 | δ = 8.36(4H, d), 8.19(2H, d), 7.79(1H, d), 7.78(1H, d), 7.76(1H, s), 7.74(1H, t), 7.62(2H, |
| | dd), 7.6(1H, d), 7.58(1H, t), 7.57(2H, t), 7.5(8H, t), 7.47(1H, d), 7.4(1H, t), 7.2(1H, t), |
| | 7.18(1H, t) |

TABLE 5

| Compound | FD-Mass | Compound | FD-Mass |
|---|---|---|---|
| 1-9 | m/z = 753.90 (C56H35NO2, | 1-358 | m/z = 677.80 (C50H31NO2, |
| | 753.27) | | 677.24) |
| 1-12 | m/z = 830.00 (C62H39NO2, | 1-369 | m/z = 783.95 (C56H33NO2S, |
| | 829.30) | | 783.22) |
| 1-20 | m/z = 793.97 (C59H39NO2, | 1-375 | m/z = 803.96 (C60H37NO2, |
| | 793.30) | | 803.28) |
| 1-29 | m/z = 843.00 (C62H38N2O2, | 1-413 | m/z = 862.12 (C62H39NS2, |
| | 842.29) | | 861.25) |
| 1-45 | m/z = 783.95 (C56H33NO2S, | 1-416 | m/z = 800.01 (C56H33NOS2, |
| | 783.22) | | 799.20) |
| 1-48 | m/z = 767.88 (C56H33NO3, | 1-417 | m/z = 826.09 (C59H39NS2, |
| | 767.25) | | 825.25) |
| 1-117 | m/z = 855.01 (C63H38N2O2, | 1-420 | m/z = 750.93 (C51H30N2OS2, |
| | 854.29) | | 750.18) |
| 1-123 | m/z = 911.09 (C65H38N2O2S, | 1-422 | m/z = 780.06 (C56H9D26NO2, |
| | 910.27) | | 779.43) |
| 1-149 | m/z = 894.04 (C66H39NO3, | 1-423 | m/z = 762.96 (C56H26D9NO2, |
| | 893.29) | | 762.32) |
| 1-154 | m/z = 906.10 (C68H43NO2, | 1-426 | m/z = 869.24 (C62D39NO2, |
| | 905.33) | | 868.54) |
| 1-179 | m/z = 803.96 (C60H37NO2, | 1-432 | m/z = 859.10 (C62H22D16N2O2, |
| | 803.28) | | 858.39) |
| 1-184 | m/z = 843.00 (C62H38N2O2, | 1-439 | m/z = 901.36 (C62D39NS2, |
| | 842.29) | | 900.50) |
| 1-221 | m/z = 767.88 (C56H33NO3, | 2-5 | m/z = 651.77 (C47H29N3O, |
| | 767.25) | | 651.23) |
| 1-218 | m/z = 677.80 (C50H31NO2, | 2-27 | m/z = 615.69 (C43H25N3O2, |
| | 677.24) | | 615.19) |
| 1-255 | m/z = 727.86 (C54H33NO2, | 2-35 | m/z = 651.77 (C47H29N3O, |
| | 727.25) | | 651.23) |
| 1-284 | m/z = 803.96 (C60H37NO2, | 2-52 | m/z = 665.75 (C47H27N3O2, |
| | 803.28) | | 665.21) |
| 1-302 | m/z = 843.00 (C62H38N2O2, | 2-84 | m/z = 615.69 (C43H25N3O2, |
| | 842.29) | | 615.19) |
| 1-313 | m/z = 843.00 (C62H38N2O2, | 2-114 | m/z = 565.63 (C39H23N3O2, |
| | 842.29) | | 565.18) |
| 1-321 | m/z = 753.90 (C56H35NO2, | 2-131 | m/z = 601.71 (C43H27N3O, |
| | 753.27) | | 601.22) |
| 1-340 | m/z = 843.00 (C62H38N2O2, | 2-169 | m/z = 640.75 (C45H28N4O, |
| | 842.29) | | 640.23) |
| 1-350 | m/z = 793.97 (C59H39NO2, | | |
| | 793.30) | | |

The contents described above are merely examples of applying the principles of the present disclosure, and other configurations may be further included without departing from the scope of the present disclosure.

### Example: Manufacture of Light-Emitting Device

A glass substrate coated with an indium tin oxide (ITO) film having a thickness of 1,500 Å was ultrasonically washed in distilled water. After completion of distilled water washing the substrate was ultrasonically washed with solvents such as acetone, methanol, and isopropyl alcohol. After drying, the substrate was subjected to ultraviolet ozone (UVO) treatment for 5 minutes in an ultraviolet (UV) cleaner. The substrate was then transferred to a plasma cleaner (PC) and subjected to plasma treatment under vacuum to adjust the ITO work function and remove any residual film. The substrate was then transferred to a thermal evaporation device for organic vapor deposition.

A common layer including a hole injection layer (2-TNATA) (4,4′,4″-tris[2-naphthyl(phenyl)amino]triphenylamine), and a hole transport layer (NPB) (N,N′-di(1-naphthyl)-N,N′-diphenyl-(1,1′-biphenyl)-4,4′-diamine), was formed on the ITO transparent electrode (anode).

A light-emitting layer was thermally vacuum-deposited on the hole transport layer as follows. Specifically, the compounds listed in Table 6 or the mixtures listed in Table 7 were used as a red host, and 3 wt % of a red phosphorescent dopant, (pig)₂Ir(acac), was doped into the red host to deposit alight-emitting layer having a thickness of 500 Å.

A 60 Å-thick BCP layer was deposited on the light-emitting layer as a hole-blocking layer, and a 200 Å-thick Alq₃ layer was deposited on the hole-blocking layer as an electron transport layer. Subsequently, a 60 Å-thick BCP layer was deposited as a hole-blocking layer, and a 200 Å-thick Alq₃ layer was deposited thereon as an electron transport layer.

An organic electroluminescent device was manufactured by depositing lithium fluoride (LiF) to a thickness of 10 Å on the electron transport layer to form an electron injection layer, followed by depositing an aluminum (Al) cathode to a thickness of 1,200 Å on the electron injection layer to form a cathode.

Meanwhile, all organic compounds required for manufacturing the OLED device were purified by vacuum sublimation at 10⁻⁶ to 10⁻⁸ torr for each material and used in the fabrication of the OLED.

The electroluminescence (EL) characteristics of the organic electroluminescent device manufactured as described above were measured using an M7000 from MaxScience. Based on these measurements, the T₉₀ value was measured using an M6000 lifetime measurement system manufactured by MaxScience at a reference luminance of 6,000 cd/m². T₉₀ refers to the lifetime (unit: h, hours), which is the time required for the luminance to decrease to 90% of the initial luminance.

The structures of the compounds used in the comparative examples in Tables 6 and 7 are as follows.

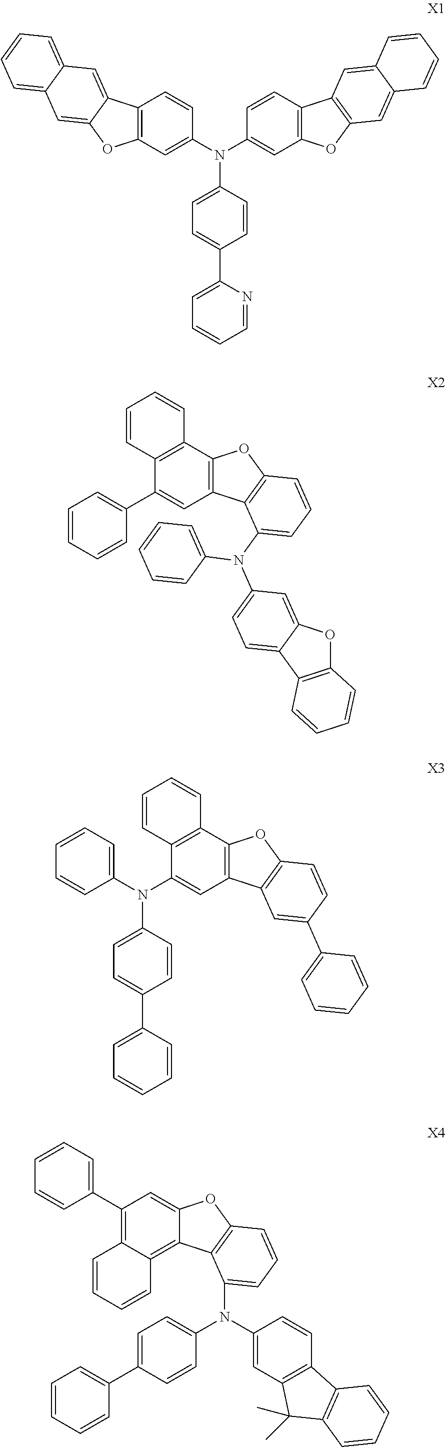 [SMILES: c1ccc(-c2ccc(N(c3ccc4c(c3)oc3cc5ccccc5cc34)c3ccc4c(c3)oc3cc5ccccc5cc34)cc2)nc1 ; c1ccc(-c2cc3c(oc4cccc(N(c5ccccc5)c5ccc6c(c5)oc5ccccc56)c43)c3ccccc23)cc1 ; c1ccc(-c2ccc(N(c3ccccc3)c3cc4c5cc(-c6ccccc6)ccc5oc4c4ccccc34)cc2)cc1 ; CC1(C)c2ccccc2-c2ccc(N(c3ccc(-c4ccccc4)cc3)c3cccc4oc5cc(-c6ccccc6)c6ccccc6c5c34)cc21]

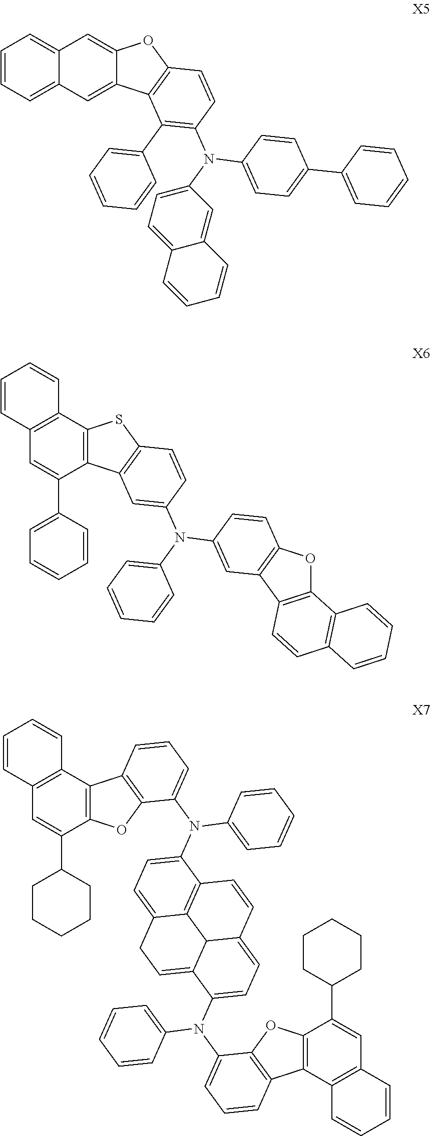 [SMILES: c1ccc(-c2ccc(N(c3ccc4ccccc4c3)c3ccc4oc5cc6ccccc6cc5c4c3-c3ccccc3)cc2)cc1 ; c1ccc(-c2cc3ccccc3c3sc4ccc(N(c5ccccc5)c5ccc6oc7c8ccccc8ccc7c6c5)cc4c23)cc1 ; C1=Cc2c(N(c3ccccc3)c3cccc4c3oc3c(C5CCCCC5)cc5ccccc5c34)ccc3c2C2C1=CC=C(N(c1ccccc1)c1cccc4c1oc1c(C5CCCCC5)cc5ccccc5c14)C2=CC3]

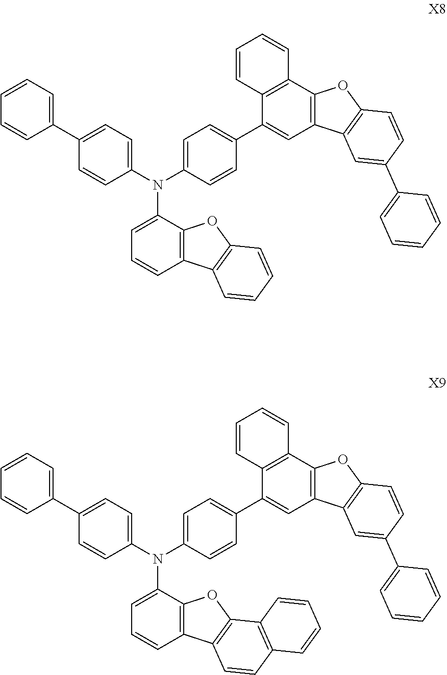 [SMILES: c1ccc(-c2ccc(N(c3ccc(-c4cc5c6cc(-c7ccccc7)ccc6oc5c5ccccc45)cc3)c3cccc4c3oc3ccccc34)cc2)cc1 ; c1ccc(-c2ccc(N(c3ccc(-c4cc5c6cc(-c7ccccc7)ccc6oc5c5ccccc45)cc3)c3cccc4c3oc3c5ccccc5ccc43)cc2)cc1]

TABLE 6

| | | Operating | Light-emitting | Color coordinates | Lifetime |
|---|---|---|---|---|---|
| | Compound | voltage (V) | efficiency (cd/A) | (x, y) | (T₉₀) |
| Example 1 | X1 | 4.21 | 23.3 | (0.686, 0.314) | 33 |
| Example 2 | X2 | 4.15 | 27.2 | (0.685, 0.315) | 53 |
| Example 3 | X3 | 4.37 | 26.1 | (0.685, 0.315) | 44 |
| Example 4 | X4 | 4.35 | 21.7 | (0.682, 0.318) | 43 |
| Example 5 | X5 | 4.26 | 22.6 | (0.684, 0.316) | 35 |
| Example 6 | X6 | 4.45 | 17.5 | (0.685, 0.315) | 19 |
| Example 7 | X7 | 4.59 | 16.2 | (0.685, 0.315) | 56 |
| Example 8 | X8 | 4.40 | 26.0 | (0.683, 0.316) | 40 |
| Example 9 | X9 | 4.38 | 26.3 | (0.684, 0.317) | 41 |
| Comparative | 1-9 | 3.84 | 39.2 | (0.682, 0.318) | 134 |
| Example 1 | | | | | |
| Comparative | 1-12 | 3.91 | 38.9 | (0.684, 0.318) | 146 |
| Example 2 | | | | | |
| Comparative | 1-20 | 3.86 | 41.2 | (0.684, 0.317) | 125 |
| Example 3 | | | | | |
| Comparative | 1-29 | 3.87 | 43.7 | (0.683, 0.316) | 130 |
| Example 4 | | | | | |
| Comparative | 1-45 | 3.71 | 37.6 | (0.685, 0.318) | 105 |
| Example 5 | | | | | |
| Comparative | 1-48 | 3.86 | 38.5 | (0.682, 0.316) | 116 |
| Example 6 | | | | | |
| Comparative | 1-117 | 3.72 | 37.1 | (0.686, 0.318) | 102 |
| Example 7 | | | | | |
| Comparative | 1-123 | 3.71 | 36.7 | (0.685, 0.318) | 92 |
| Example 8 | | | | | |
| Comparative | 1-149 | 3.72 | 36.2 | (0.682, 0.318) | 94 |
| Example 9 | | | | | |
| Comparative | 1-154 | 3.87 | 38.2 | (0.683, 0.317) | 131 |
| Example 10 | | | | | |
| Comparative | 1-179 | 4.04 | 35.0 | (0.685, 0.315) | 95 |
| Example 11 | | | | | |
| Comparative | 1-184 | 4.01 | 35.5 | (0.682, 0.315) | 97 |
| Example 12 | | | | | |
| Comparative | 1-221 | 3.89 | 35.8 | (0.683, 0.318) | 102 |
| Example 13 | | | | | |
| Comparative | 1-218 | 4.05 | 37.0 | (0.683, 0.317) | 126 |
| Example 14 | | | | | |
| Comparative | 1-255 | 3.98 | 34.4 | (0.682, 0.317) | 79 |
| Example 15 | | | | | |
| Comparative | 1-284 | 3.97 | 33.6 | (0.684, 0.318) | 78 |
| Example 16 | | | | | |
| Comparative | 1-302 | 3.90 | 34.1 | (0.683, 0.317) | 87 |
| Example 17 | | | | | |
| Comparative | 1-313 | 3.80 | 32.8 | (0.682, 0.316) | 109 |
| Example 18 | | | | | |
| Comparative | 1-321 | 3.78 | 33.4 | (0.683, 0.317) | 129 |
| Example 19 | | | | | |
| Comparative | 1-340 | 3.85 | 32.7 | (0.684, 0.317) | 110 |
| Example 20 | | | | | |
| Comparative | 1-350 | 3.88 | 32.1 | (0.683, 0.316) | 125 |
| Example 21 | | | | | |
| Comparative | 1-358 | 3.77 | 30.8 | (0.682, 0.315) | 87 |
| Example 22 | | | | | |
| Comparative | 1-369 | 3.75 | 30.4 | (0.683, 0.318) | 77 |
| Example 23 | | | | | |
| Comparative | 1-375 | 3.69 | 31.9 | (0.684, 0.317) | 106 |
| Example 24 | | | | | |
| Comparative | 1-413 | 3.90 | 29.8 | (0.684, 0.318) | 70 |
| Example 25 | | | | | |
| Comparative | 1-416 | 3.85 | 29.1 | (0.683, 0.317) | 68 |
| Example 26 | | | | | |
| Comparative | 1-417 | 3.88 | 28.6 | (0.683, 0.316) | 65 |
| Example 27 | | | | | |
| Comparative | 1-420 | 3.87 | 28.2 | (0.684, 0.318) | 60 |
| Example 28 | | | | | |
| Comparative | 1-422 | 3.85 | 39.2 | (0.683, 0.317) | 205 |
| Example 29 | | | | | |
| Comparative | 1-423 | 3.84 | 39.1 | (0.682, 0.318) | 165 |
| Example 30 | | | | | |
| Comparative | 1-426 | 3.91 | 38.9 | (0.682, 0.316) | 116 |
| Example 31 | | | | | |
| Comparative | 1-432 | 3.87 | 43.7 | (0.683, 0.317) | 159 |
| Example 32 | | | | | |
| Comparative | 1-439 | 3.90 | 29.8 | (0.685, 0.318) | 140 |
| Example 33 | | | | | |

TABLE 7

| | Host mixture | | Light-emitting | | |
|---|---|---|---|---|---|
| | (type = weight | Operating | efficiency | Color coordinates | Lifetime |
| | ratio) | voltage (V) | (cd/A) | (x, y) | (T90) |
| Example 10 | X1:2-27 = 1:1 | 4.20 | 28.9 | (0.684, 0.317) | 78 |
| Example 11 | X1:2-27 = 1.5:1 | 4.32 | 27.7 | (0.683, 0.316) | 75 |
| Example 12 | X1:2-27 = 1:1.5 | 4.11 | 30.3 | (0.685, 0.318) | 80 |
| Example 13 | X1:2-52 = 1:1.5 | 4.26 | 28.0 | (0.682, 0.316) | 81 |
| Example 14 | X1:2-131 = 1:1.5 | 4.19 | 29.6 | (0.686, 0.318) | 78 |
| Example 15 | X2:2-27 = 1:1.5 | 4.05 | 33.0 | (0.683, 0.317) | 96 |
| Example 16 | X2:2-52 = 1:1.5 | 4.20 | 32.6 | (0.683, 0.317) | 99 |
| Comparative | 1-9:2-52 = 1:1.5 | 3.79 | 47.0 | (0.682, 0.318) | 218 |
| Example 34 | | | | | |
| Comparative | 1-12:2-5 = 1:1.5 | 3.81 | 45.9 | (0.682, 0.315) | 223 |
| Example 35 | | | | | |
| Comparative | 1-20:2-5 = 1:1.5 | 3.82 | 48.6 | (0.684, 0.317) | 200 |
| Example 36 | | | | | |
| Comparative | 1-20:2-27 = 1:1.5 | 3.76 | 53.6 | (0.683, 0.316) | 198 |
| Example 37 | | | | | |
| Comparative | 1-20:2-35 = 1:1.5 | 3.96 | 51.5 | (0.685, 0.318) | 182 |
| Example 38 | | | | | |
| Comparative | 1-20:2-52 = 1:1.5 | 3.91 | 49.4 | (0.682, 0.318) | 206 |
| Example 39 | | | | | |
| Comparative | 1-20:2-84 = 1:1.5 | 3.79 | 45.3 | (0.683, 0.317) | 201 |
| Example 40 | | | | | |
| Comparative | 1-20:2-114 = 1:1.5 | 3.84 | 51.5 | (0.682, 0.316) | 188 |
| Example 41 | | | | | |
| Comparative | 1-20:2-131 = 1:1.5 | 3.84 | 52.3 | (0.683, 0.317) | 191 |
| Example 42 | | | | | |
| Comparative | 1-20:2-169 = 1:1.5 | 3.79 | 47.4 | (0.682, 0.318) | 190 |
| Example 43 | | | | | |
| Comparative | 1-29:2-27 = 1:1.5 | 3.7 | 56.8 | (0.683, 0.317) | 204 |
| Example 44 | | | | | |
| Comparative | 1-29:2-52 = 1:1.5 | 3.82 | 52.4 | (0.682, 0.316) | 213 |
| Example 45 | | | | | |
| Comparative | 1-45:2-52 = 1:1.5 | 3.66 | 45.1 | (0.682, 0.315) | 181 |
| Example 46 | | | | | |
| Comparative | 1-48:2-52 = 1:1.5 | 3.81 | 46.2 | (0.682, 0.316) | 195 |
| Example 47 | | | | | |
| Comparative | 1-117:2-27 = 1:1.5 | 3.62 | 48.2 | (0.683, 0.317) | 170 |
| Example 48 | | | | | |
| Comparative | 1-123:2-27 = 1:1.5 | 3.61 | 47.7 | (0.686, 0.314) | 158 |
| Example 49 | | | | | |
| Comparative | 1-149:2-27 = 1:1.5 | 3.62 | 47.1 | (0.684, 0.318) | 161 |
| Example 50 | | | | | |
| Comparative | 1-154:2-52 = 1:1.5 | 3.82 | 45.8 | (0.685, 0.315) | 214 |
| Example 51 | | | | | |
| Comparative | 1-179:2-52 = 1:1.5 | 3.99 | 42.0 | (0.682, 0.318) | 169 |
| Example 52 | | | | | |
| Comparative | 1-184:2-52 = 1:1.5 | 3.96 | 42.6 | (0.682, 0.318) | 171 |
| Example 53 | | | | | |
| Comparative | 1-221:2-52 = 1:1.5 | 3.84 | 43.0 | (0.683, 0.317) | 178 |
| Example 54 | | | | | |
| Comparative | 1-218:2-52 = 1:1.5 | 4.00 | 44.4 | (0.684, 0.315) | 208 |
| Example 55 | | | | | |
| Comparative | 1-255:2-27 = 1:1.5 | 3.88 | 44.7 | (0.683, 0.317) | 143 |
| Example 56 | | | | | |
| Comparative | 1-284:2-27 = 1:1.5 | 3.87 | 43.7 | (0.682, 0.318) | 142 |
| Example 57 | | | | | |
| Comparative | 1-302:2-27 = 1:1.5 | 3.80 | 44.3 | (0.682, 0.318) | 151 |
| Example 58 | | | | | |
| Comparative | 1-313:2-52 = 1:1.5 | 3.75 | 39.4 | (0.683, 0.317) | 186 |
| Example 59 | | | | | |
| Comparative | 1-321:2-52 = 1:1.5 | 3.73 | 40.1 | (0.682, 0.318) | 211 |
| Example 60 | | | | | |
| Comparative | 1-340:2-52 = 1:1.5 | 3.80 | 39.2 | (0.685, 0.315) | 188 |
| Example 61 | | | | | |
| Comparative | 1-350:2-52 = 1:1.5 | 3.83 | 38.5 | (0.683, 0.317) | 206 |
| Example 62 | | | | | |
| Comparative | 1-358:2-27 = 1:1.5 | 3.67 | 40.0 | (0.683, 0.317) | 152 |
| Example 63 | | | | | |
| Comparative | 1-369:2-52 = 1:1.5 | 3.70 | 36.5 | (0.683, 0.317) | 146 |
| Example 64 | | | | | |
| Comparative | 1-375:2-52 = 1:1.5 | 3.64 | 38.3 | (0.682, 0.318) | 183 |
| Example 65 | | | | | |
| Comparative | 1-413:2-52 = 1:1.5 | 3.85 | 35.8 | (0.685, 0.315) | 115 |
| Example 66 | | | | | |
| Comparative | 1-416:2-52 = 1:1.5 | 3.80 | 34.9 | (0.683, 0.317) | 112 |
| Example 67 | | | | | |
| Comparative | 1-417:2-52 = 1:1.5 | 3.83 | 34.3 | (0.686, 0.314) | 107 |
| Example 68 | | | | | |
| Comparative | 1-420:2-52 = 1:1.5 | 3.84 | 33.8 | (0.685, 0.315) | 105 |
| Example 69 | | | | | |
| Comparative | 1-422:2-27 = 1:1.5 | 3.75 | 51.0 | (0.682, 0.318) | 246 |
| Example 70 | | | | | |
| Comparative | 1-423:2-27 = 1:1.5 | 3.74 | 50.8 | (0.686, 0.314) | 240 |
| Example 71 | | | | | |
| Comparative | 1-426:2-52 = 1:1.5 | 3.86 | 46.7 | (0.684, 0.318) | 320 |
| Example 72 | | | | | |
| Comparative | 1-432:2-27 = 1:1.5 | 3.77 | 56.8 | (0.683, 0.317) | 239 |
| Example 73 | | | | | |
| Comparative | 1-439:2-27 = 1:1.5 | 3.85 | 38.7 | (0.683, 0.317) | 221 |
| Example 74 | | | | | |

The heterocyclic compound of Formula 1 exhibits high thermal stability while possessing an appropriate molecular weight and band gap. The appropriate band gap of the light-emitting layer including the compound may prevent the loss of electrons and holes and effectively form a recombination zone.

Referring to Table 6, the organic light-emitting device using the compound of Formula 1 exhibited improved physical properties compared to the organic light-emitting devices using Compounds X1 to X7 of the comparative examples. This improvement is considered to result from enhanced hole mobility due to the expanded conjugation of Rₐ and R_{b} having a naphtho-benzofuran structure.

The light-emitting devices of the examples exhibited T₉₀ lifetimes of 60 hours or more, particularly 100 hours or more, light-emitting efficiencies of 28.2 cd/A or higher, and operating voltages of 4.05 V or lower. On the other hand, the light-emitting devices of the comparative examples exhibited inferior properties, such as T₉₀ lifetimes of 56 hours or less, light-emitting efficiencies of 27.2 cd/A or lower, and operating voltages of 4.15 V or higher than the light-emitting devices of the examples.

Referring to Table 7, when a combination of the heterocyclic compound of Formula 1 and the compound of Formula 3 was used as a host in the light-emitting layer, the device exhibited improved operating voltage, light-emitting efficiency, and lifetime characteristics compared to using each compound individually.

The light-emitting layers of the organic light-emitting devices of the examples include the heterocyclic compound of Formula 1 (p-host) as a donor with strong hole transport capability and the heterocyclic compound of Formula 3 (n-host) as an acceptor with strong electron transport capability. As a result, the operating voltage for electron and hole injection may be reduced, and the recombination zone may be formed more effectively, thereby further improving the light-emitting efficiency and lifetime of the light-emitting device.

The light-emitting devices of the examples exhibited T₉₀ lifetimes of 105 hours or more, particularly 160 hours or more, light-emitting efficiencies of 33.8 cd/A or higher, and operating voltages of 4.0 V or lower. On the other hand, the light-emitting devices of the comparative examples exhibited inferior properties, with T₉₀ lifetimes of 106 hours or less, light-emitting efficiencies of 33.0 cd/A or lower, and operating voltages of 4.05 V or higher, as compared to the light-emitting devices of the examples.

What is claimed is:

1. A heterocyclic compound represented by Formula 1: 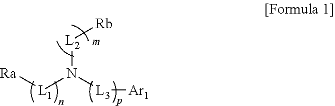
wherein, in Formula 1, Rₐ and R_{b} are each independently represented by Formula 2: 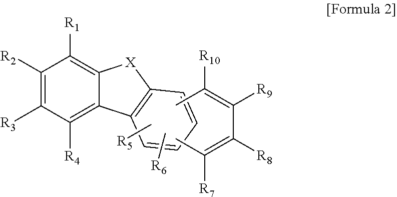
wherein, in Formula 2, one of R₁ to R₁₀ is -(L₄)_{q}-Ar₂, another of R₁ to R₁₀ represents a bonding site with L₁ or L₂ of Formula 1, and the others of R₁ to R₁₀ are each independently selected from the group consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein adjacent groups are linked to form a substituted or unsubstituted 5- to 9-membered saturated or unsaturated ring,
R, R′ and R″ are each independently hydrogen, deuterium, —CN, a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms,
X is O or S, and
in Formulae 1 and 2, L₁ to L₄ are each independently a direct linkage, a substituted or unsubstituted arylene group having 6 to 60 carbon atoms, or a substituted or unsubstituted heteroarylene group having 2 to 60 carbon atoms,
Ar₁ and Ar₂ are each independently a substituted or unsubstituted aryl group having 6 to 60 carbon atoms, or a substituted or unsubstituted heteroaryl group having 2 to 60 carbon atoms,
n, m, p, and q are each independently 0 or an integer of 1 to 5, and
when n, m, p, and q are each 2 or more, a plurality of L₁ groups to L₄ groups are each the same or different.

2. The heterocyclic compound according to claim 1, wherein, in Formula 1, p is 0, 1, or 2, and Ar₁ is a substituted or unsubstituted aryl group having 6 to 40 carbon atoms, or a substituted or unsubstituted heteroaryl group having 2 to 40 carbon atoms.

3. The heterocyclic compound according to claim 1, wherein, in Formula 1, Rₐ and R_{b} are each independently represented by any one of Formulae 2-1 to 2-3: 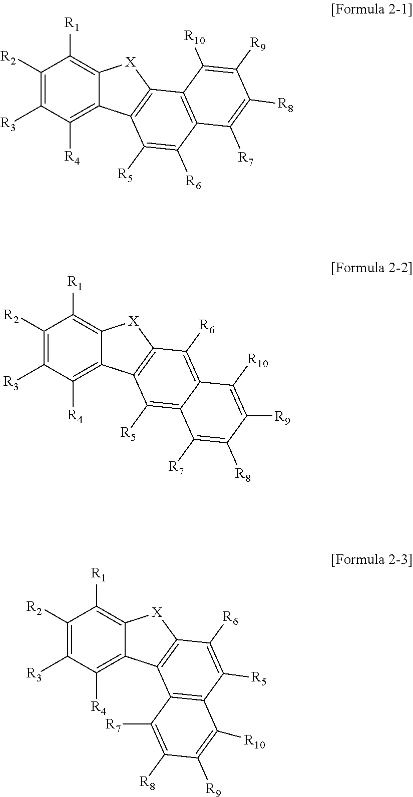
wherein, in Formulae 2-1 to 2-3, the definitions of X and R₁ to R₁₀ are the same as defined in claim 1.

4. The heterocyclic compound according to claim 1, wherein, in Formula 1, Rₐ and R_{b} are each independently represented by any one of Formulae 2-1-1 to 2-1-11, Formulae 2-2-1 to 2-2-6, and Formulae 2-3-1 to 2-3-5: 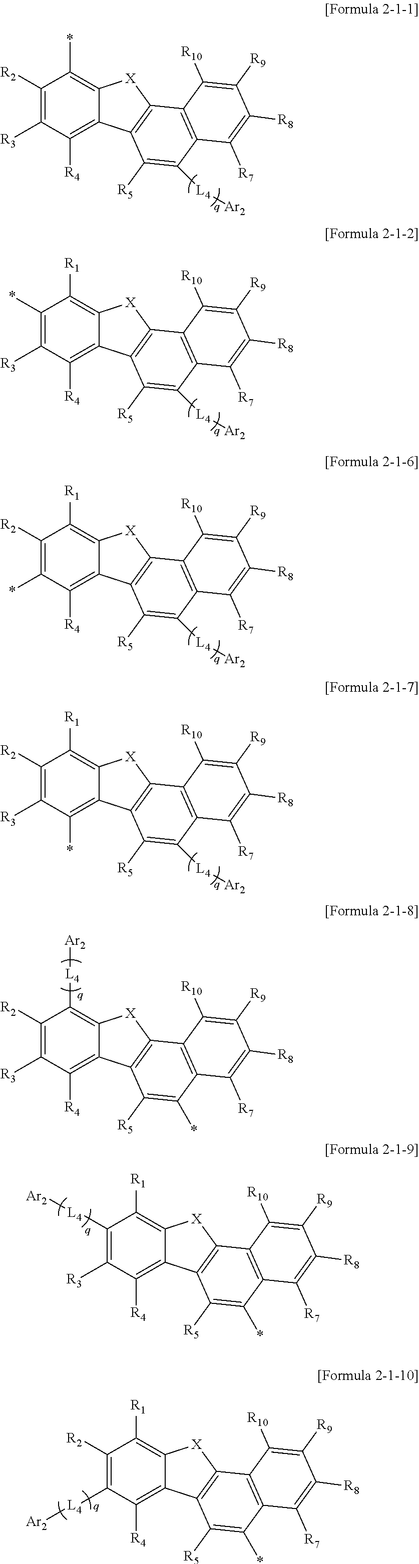 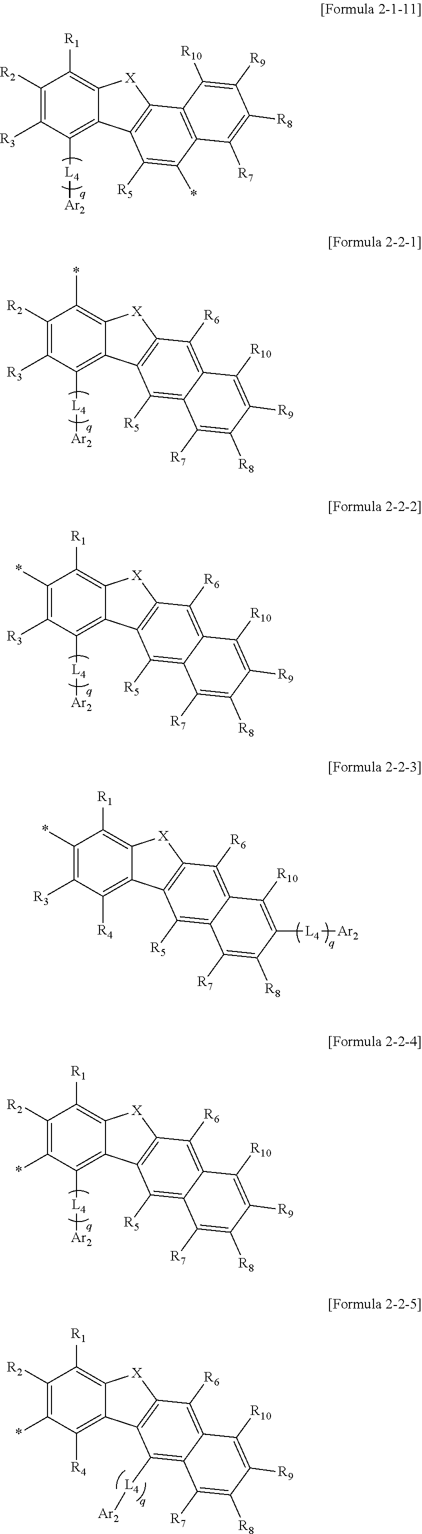 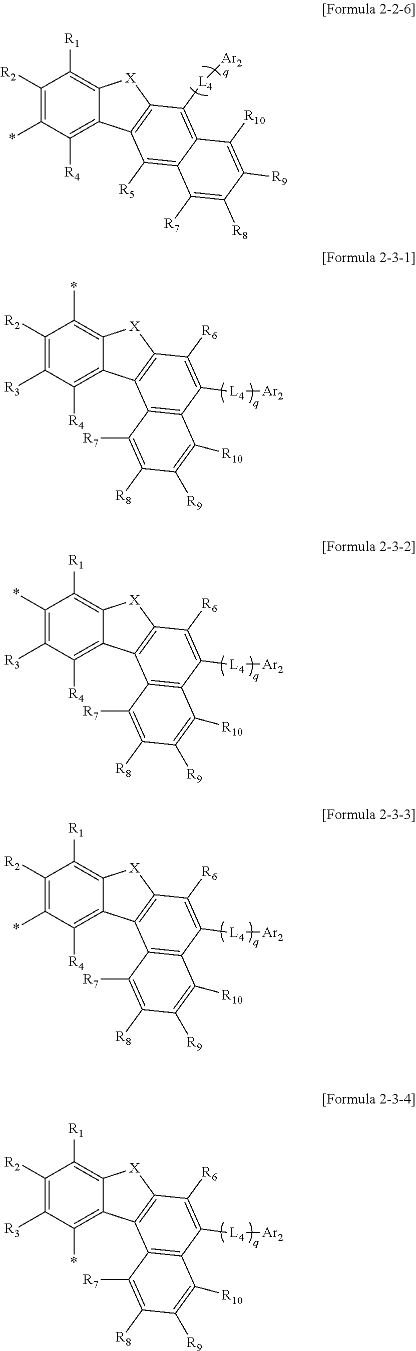 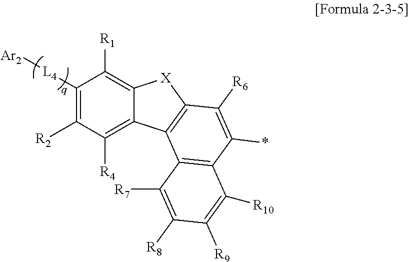
wherein, in Formulae 2-1-1 to 2-1-11, Formulae 2-2-1 to 2-2-6, and Formulae 2-3-1 to 2-3-5, * represents a bonding site, and the definitions of X, R₁ to R₁₀, L₄, Ar₂, and q are the same as defined in claim 1.

5. The heterocyclic compound according to claim 4, wherein, in each of Formulae 2-1-1 to 2-1-11, Formulae 2-2-1 to 2-2-6, and Formulae 2-3-1 to 2-3-5, q is 0, 1, or 2, and Ar₂ is independently a substituted or unsubstituted aryl group having 6 to 40 carbon atoms.

6. The heterocyclic compound according to claim 1, wherein the heterocyclic compound is represented by any one of Formulae 1-2 to 1-4: 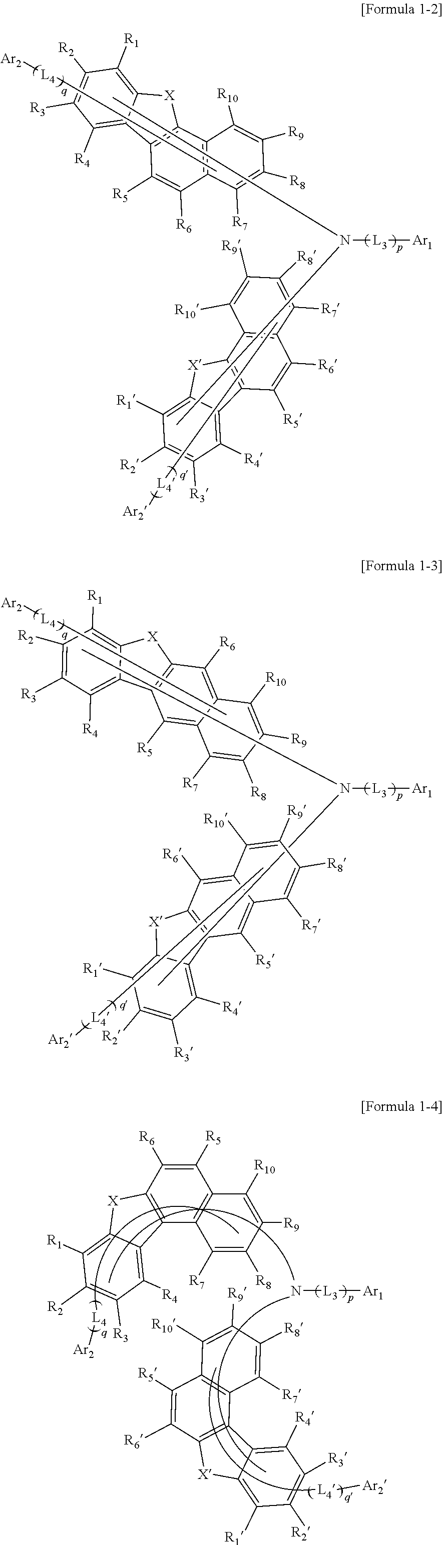
wherein, in Formulae 1-2 to 1-4, the definitions of X, L₃, L₄, Ar₁, Ar₂, p, and q are the same as defined in claim 1,
X′ is O or S,
one of R₁ to R₁₀ represents a bonding site with L4, and another of R₁ to R₁₀ represents a bonding site with N,
one of R₁′ to R₁₀′ represents a bonding site with L4′, and another R₁′ to R₁₀′ represents a bonding site with N,
the substituents other than those serving as bonding sites with L₄, L₄′, or N among R₁ to R₁₀ and R₁′ to R₁₀′ are each independently selected from the group consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein adjacent groups are linked to form a substituted or unsubstituted 5- to 9-membered saturated or unsaturated ring,
R, R′, and R″ are each independently hydrogen, deuterium, —CN, a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms,
Ar₂ is a substituted or unsubstituted aryl group having 6 to 60 carbon atoms, or a substituted or unsubstituted heteroaryl group having 2 to 60 carbon atoms, and
L₄′ is a direct linkage, a substituted or unsubstituted arylene group having 6 to 60 carbon atoms, or a substituted or unsubstituted heteroarylene group having 2 to 60 carbon atoms,
q′ is 0 or an integer of 1 to 5, and
when q′ is 2 or more, a plurality of L₄′ groups are the same or different.

7. The heterocyclic compound according to claim 1, wherein the deuterium content is 0, or greater than 0 and not more than 100%.

8. The heterocyclic compound according to claim 1, wherein the heterocyclic compound is represented by any one of formulae below:

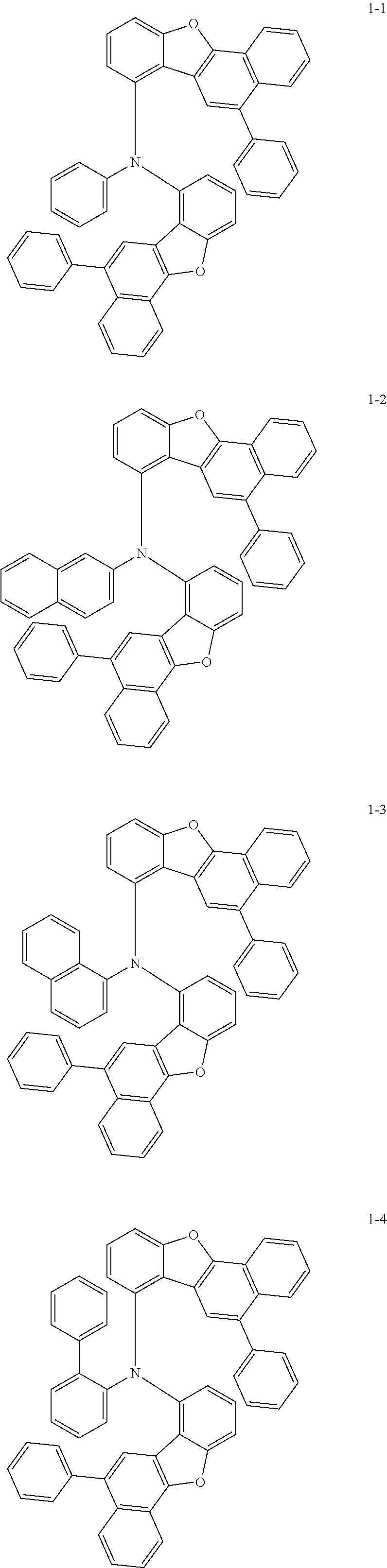 [SMILES: c1ccc(-c2cc3c(oc4cccc(N(c5ccccc5)c5cccc6oc7c8ccccc8c(-c8ccccc8)cc7c56)c43)c3ccccc23)cc1 ; c1ccc(-c2cc3c(oc4cccc(N(c5ccc6ccccc6c5)c5cccc6oc7c8ccccc8c(-c8ccccc8)cc7c56)c43)c3ccccc23)cc1 ; c1ccc(-c2cc3c(oc4cccc(N(c5cccc6ccccc56)c5cccc6oc7c8ccccc8c(-c8ccccc8)cc7c56)c43)c3ccccc23)cc1 ; c1ccc(-c2ccccc2N(c2cccc3oc4c5ccccc5c(-c5ccccc5)cc4c23)c2cccc3oc4c5ccccc5c(-c5ccccc5)cc4c23)cc1]

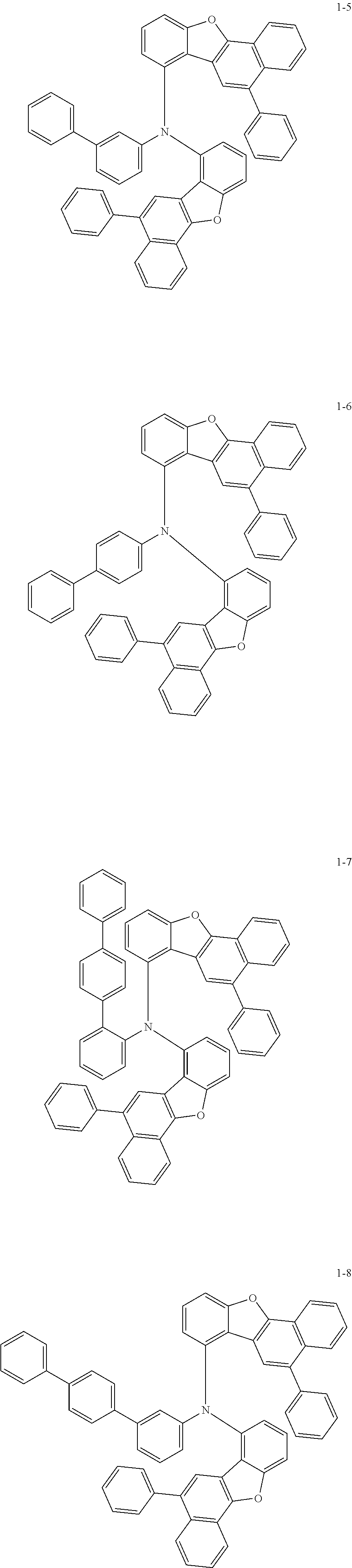 [SMILES: c1ccc(-c2cccc(N(c3cccc4oc5c6ccccc6c(-c6ccccc6)cc5c34)c3cccc4oc5c6ccccc6c(-c6ccccc6)cc5c34)c2)cc1 ; c1ccc(-c2ccc(N(c3cccc4oc5c6ccccc6c(-c6ccccc6)cc5c34)c3cccc4oc5c6ccccc6c(-c6ccccc6)cc5c34)cc2)cc1 ; c1ccc(-c2ccc(-c3ccccc3N(c3cccc4oc5c6ccccc6c(-c6ccccc6)cc5c34)c3cccc4oc5c6ccccc6c(-c6ccccc6)cc5c34)cc2)cc1 ; c1ccc(-c2ccc(-c3cccc(N(c4cccc5oc6c7ccccc7c(-c7ccccc7)cc6c45)c4cccc5oc6c7ccccc7c(-c7ccccc7)cc6c45)c3)cc2)cc1]

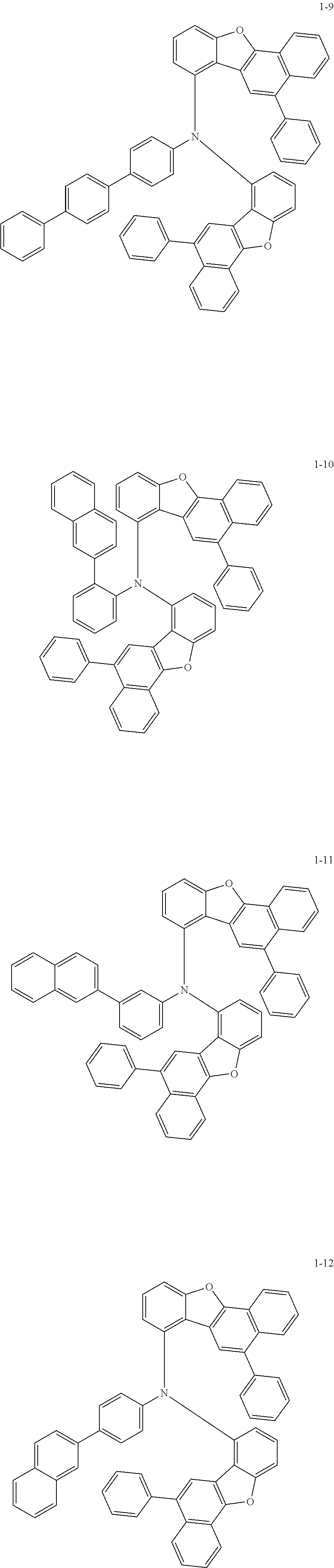 [SMILES: c1ccc(-c2ccc(-c3ccc(N(c4cccc5oc6c7ccccc7c(-c7ccccc7)cc6c45)c4cccc5oc6c7ccccc7c(-c7ccccc7)cc6c45)cc3)cc2)cc1 ; c1ccc(-c2cc3c(oc4cccc(N(c5ccccc5-c5ccc6ccccc6c5)c5cccc6oc7c8ccccc8c(-c8ccccc8)cc7c56)c43)c3ccccc23)cc1 ; c1ccc(-c2cc3c(oc4cccc(N(c5cccc(-c6ccc7ccccc7c6)c5)c5cccc6oc7c8ccccc8c(-c8ccccc8)cc7c56)c43)c3ccccc23)cc1 ; c1ccc(-c2cc3c(oc4cccc(N(c5ccc(-c6ccc7ccccc7c6)cc5)c5cccc6oc7c8ccccc8c(-c8ccccc8)cc7c56)c43)c3ccccc23)cc1]

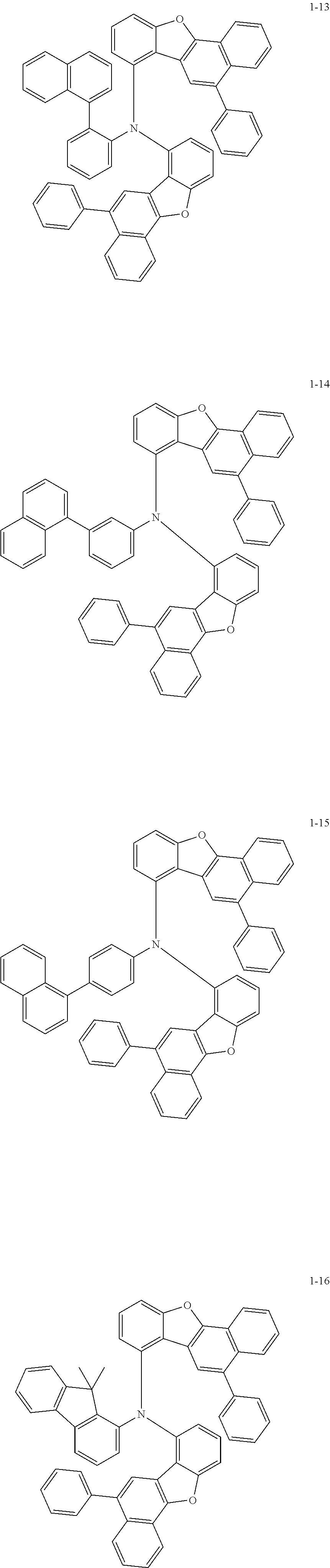 [SMILES: c1ccc(-c2cc3c(oc4cccc(N(c5ccccc5-c5cccc6ccccc56)c5cccc6oc7c8ccccc8c(-c8ccccc8)cc7c56)c43)c3ccccc23)cc1 ; c1ccc(-c2cc3c(oc4cccc(N(c5cccc(-c6cccc7ccccc67)c5)c5cccc6oc7c8ccccc8c(-c8ccccc8)cc7c56)c43)c3ccccc23)cc1 ; c1ccc(-c2cc3c(oc4cccc(N(c5ccc(-c6cccc7ccccc67)cc5)c5cccc6oc7c8ccccc8c(-c8ccccc8)cc7c56)c43)c3ccccc23)cc1 ; CC1(C)c2ccccc2-c2cccc(N(c3cccc4oc5c6ccccc6c(-c6ccccc6)cc5c34)c3cccc4oc5c6ccccc6c(-c6ccccc6)cc5c34)c21]

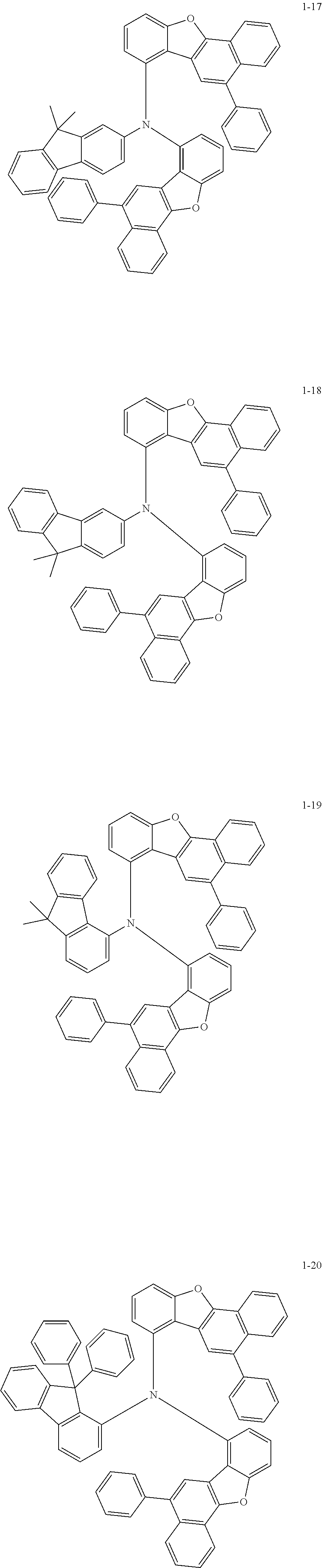 [SMILES: CC1(C)c2ccccc2-c2ccc(N(c3cccc4oc5c6ccccc6c(-c6ccccc6)cc5c34)c3cccc4oc5c6ccccc6c(-c6ccccc6)cc5c34)cc21 ; CC1(C)c2ccccc2-c2cc(N(c3cccc4oc5c6ccccc6c(-c6ccccc6)cc5c34)c3cccc4oc5c6ccccc6c(-c6ccccc6)cc5c34)ccc21 ; CC1(C)c2ccccc2-c2c(N(c3cccc4oc5c6ccccc6c(-c6ccccc6)cc5c34)c3cccc4oc5c6ccccc6c(-c6ccccc6)cc5c34)cccc21 ; c1ccc(-c2cc3c(oc4cccc(N(c5cccc6c5C(c5ccccc5)(c5ccccc5)c5ccccc5-6)c5cccc6oc7c8ccccc8c(-c8ccccc8)cc7c56)c43)c3ccccc23)cc1]

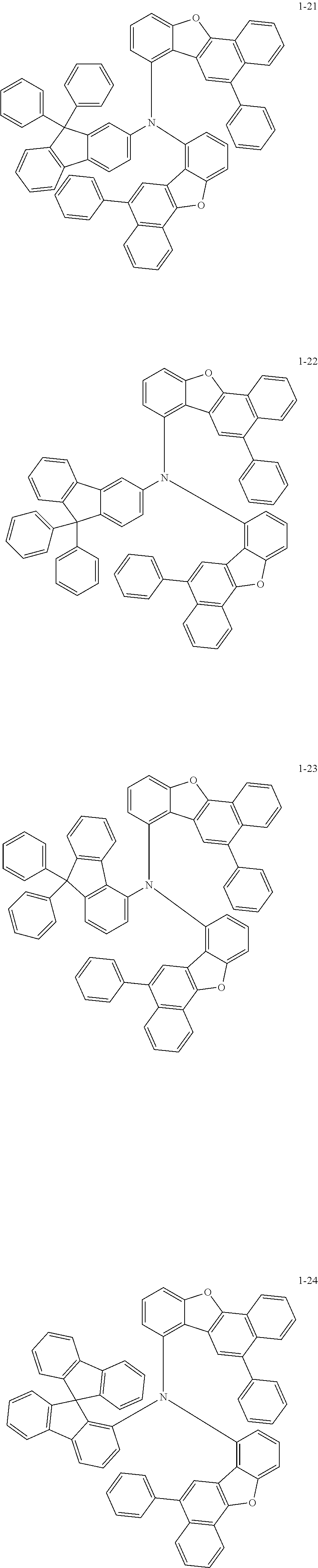 [SMILES: c1ccc(-c2cc3c(oc4cccc(N(c5ccc6c(c5)C(c5ccccc5)(c5ccccc5)c5ccccc5-6)c5cccc6oc7c8ccccc8c(-c8ccccc8)cc7c56)c43)c3ccccc23)cc1 ; c1ccc(-c2cc3c(oc4cccc(N(c5ccc6c(c5)-c5ccccc5C6(c5ccccc5)c5ccccc5)c5cccc6oc7c8ccccc8c(-c8ccccc8)cc7c56)c43)c3ccccc23)cc1 ; c1ccc(-c2cc3c(oc4cccc(N(c5cccc6c5-c5ccccc5C6(c5ccccc5)c5ccccc5)c5cccc6oc7c8ccccc8c(-c8ccccc8)cc7c56)c43)c3ccccc23)cc1 ; c1ccc(-c2cc3c(oc4cccc(N(c5cccc6c5C5(c7ccccc7-c7ccccc75)c5ccccc5-6)c5cccc6oc7c8ccccc8c(-c8ccccc8)cc7c56)c43)c3ccccc23)cc1]

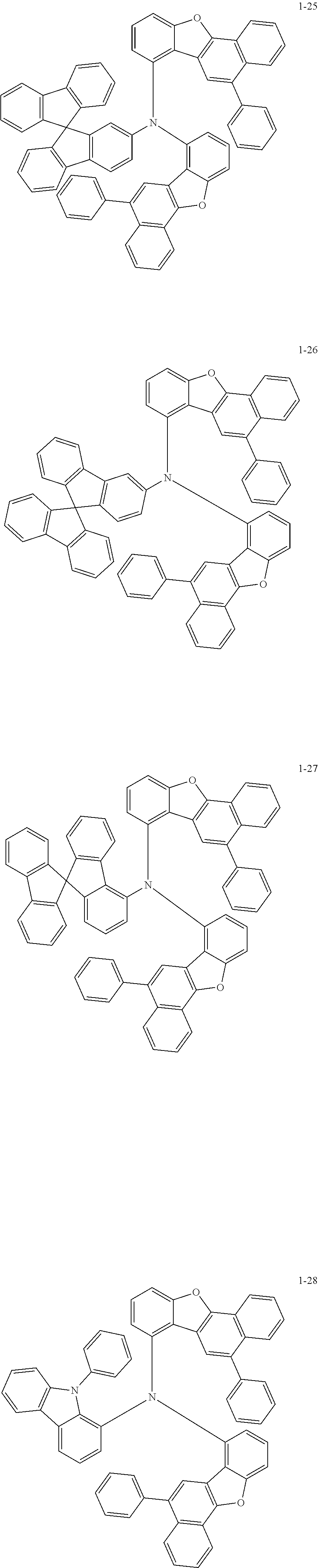 [SMILES: c1ccc(-c2cc3c(oc4cccc(N(c5ccc6c(c5)C5(c7ccccc7-c7ccccc75)c5ccccc5-6)c5cccc6oc7c8ccccc8c(-c8ccccc8)cc7c56)c43)c3ccccc23)cc1 ; c1ccc(-c2cc3c(oc4cccc(N(c5ccc6c(c5)-c5ccccc5C65c6ccccc6-c6ccccc65)c5cccc6oc7c8ccccc8c(-c8ccccc8)cc7c56)c43)c3ccccc23)cc1 ; c1ccc(-c2cc3c(oc4cccc(N(c5cccc6c5-c5ccccc5C65c6ccccc6-c6ccccc65)c5cccc6oc7c8ccccc8c(-c8ccccc8)cc7c56)c43)c3ccccc23)cc1 ; c1ccc(-c2cc3c(oc4cccc(N(c5cccc6oc7c8ccccc8c(-c8ccccc8)cc7c56)c5cccc6c7ccccc7n(-c7ccccc7)c56)c43)c3ccccc23)cc1]

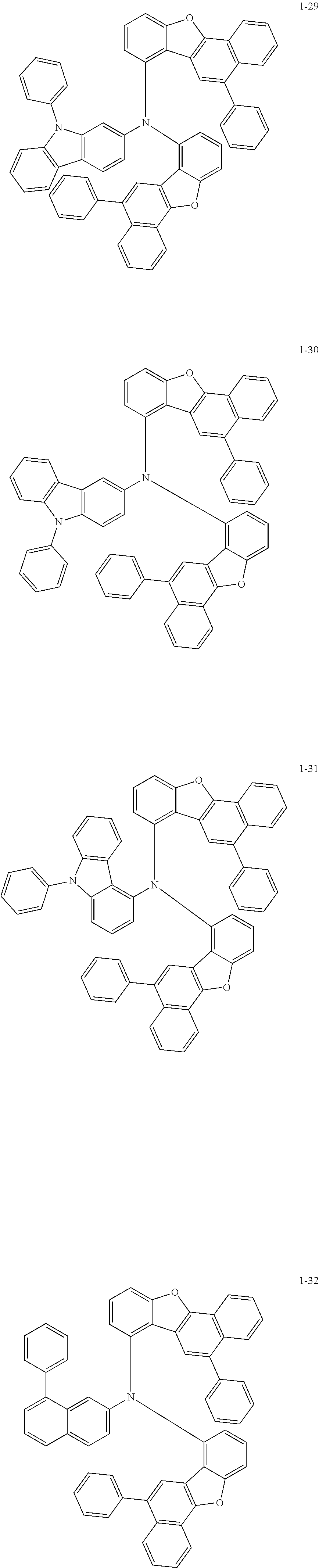 [SMILES: c1ccc(-c2cc3c(oc4cccc(N(c5ccc6c7ccccc7n(-c7ccccc7)c6c5)c5cccc6oc7c8ccccc8c(-c8ccccc8)cc7c56)c43)c3ccccc23)cc1 ; c1ccc(-c2cc3c(oc4cccc(N(c5ccc6c(c5)c5ccccc5n6-c5ccccc5)c5cccc6oc7c8ccccc8c(-c8ccccc8)cc7c56)c43)c3ccccc23)cc1 ; c1ccc(-c2cc3c(oc4cccc(N(c5cccc6oc7c8ccccc8c(-c8ccccc8)cc7c56)c5cccc6c5c5ccccc5n6-c5ccccc5)c43)c3ccccc23)cc1 ; c1ccc(-c2cccc3ccc(N(c4cccc5oc6c7ccccc7c(-c7ccccc7)cc6c45)c4cccc5oc6c7ccccc7c(-c7ccccc7)cc6c45)cc23)cc1]

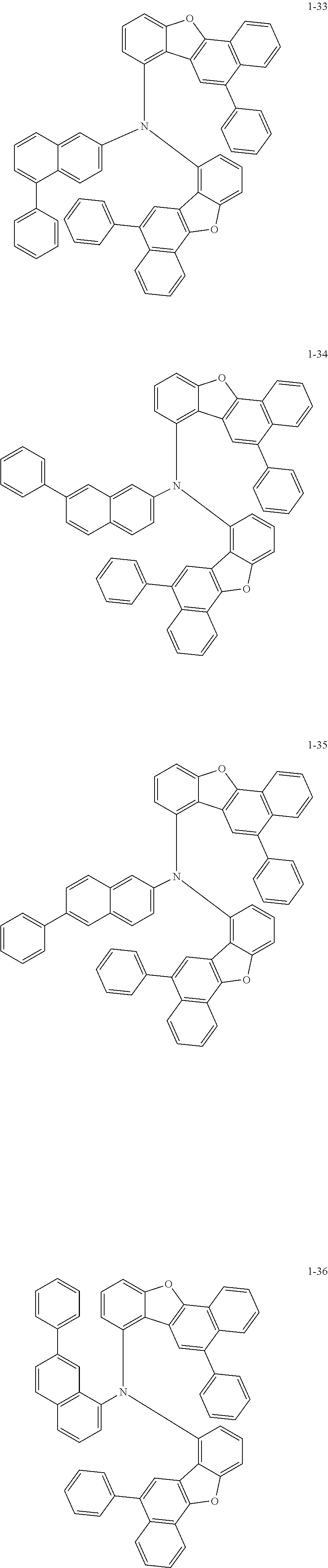 [SMILES: c1ccc(-c2cccc3cc(N(c4cccc5oc6c7ccccc7c(-c7ccccc7)cc6c45)c4cccc5oc6c7ccccc7c(-c7ccccc7)cc6c45)ccc23)cc1 ; c1ccc(-c2ccc3ccc(N(c4cccc5oc6c7ccccc7c(-c7ccccc7)cc6c45)c4cccc5oc6c7ccccc7c(-c7ccccc7)cc6c45)cc3c2)cc1 ; c1ccc(-c2ccc3cc(N(c4cccc5oc6c7ccccc7c(-c7ccccc7)cc6c45)c4cccc5oc6c7ccccc7c(-c7ccccc7)cc6c45)ccc3c2)cc1 ; c1ccc(-c2ccc3cccc(N(c4cccc5oc6c7ccccc7c(-c7ccccc7)cc6c45)c4cccc5oc6c7ccccc7c(-c7ccccc7)cc6c45)c3c2)cc1]

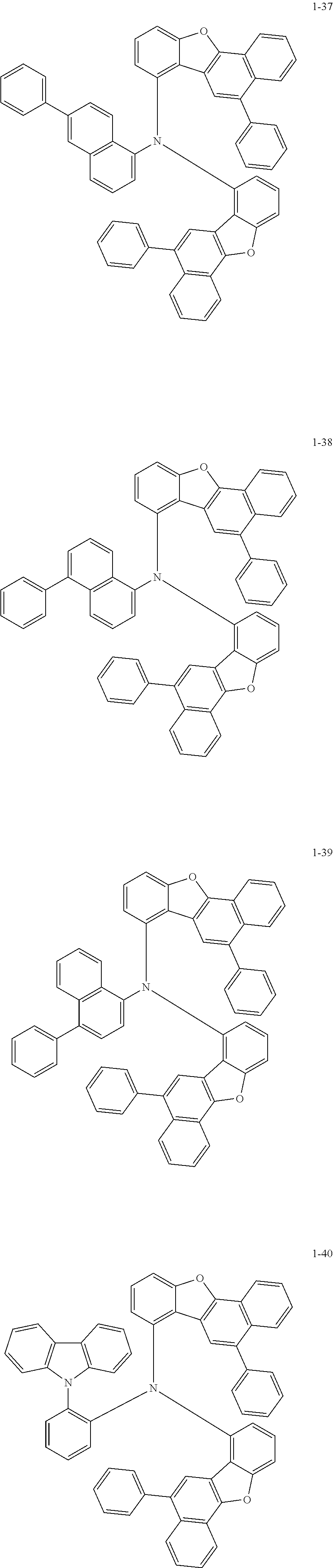 [SMILES: c1ccc(-c2ccc3c(N(c4cccc5oc6c7ccccc7c(-c7ccccc7)cc6c45)c4cccc5oc6c7ccccc7c(-c7ccccc7)cc6c45)cccc3c2)cc1 ; c1ccc(-c2cccc3c(N(c4cccc5oc6c7ccccc7c(-c7ccccc7)cc6c45)c4cccc5oc6c7ccccc7c(-c7ccccc7)cc6c45)cccc23)cc1 ; c1ccc(-c2ccc(N(c3cccc4oc5c6ccccc6c(-c6ccccc6)cc5c34)c3cccc4oc5c6ccccc6c(-c6ccccc6)cc5c34)c3ccccc23)cc1 ; c1ccc(-c2cc3c(oc4cccc(N(c5ccccc5-n5c6ccccc6c6ccccc65)c5cccc6oc7c8ccccc8c(-c8ccccc8)cc7c56)c43)c3ccccc23)cc1]

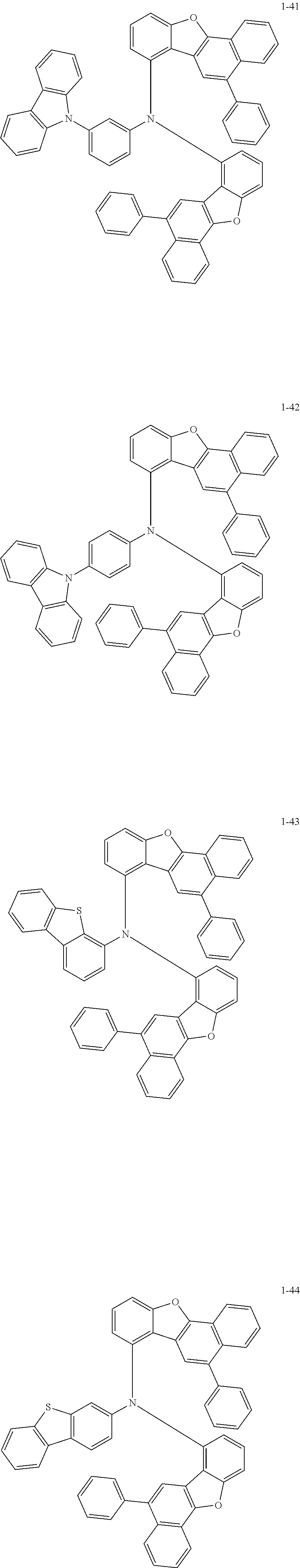 [SMILES: c1ccc(-c2cc3c(oc4cccc(N(c5cccc(-n6c7ccccc7c7ccccc76)c5)c5cccc6oc7c8ccccc8c(-c8ccccc8)cc7c56)c43)c3ccccc23)cc1 ; c1ccc(-c2cc3c(oc4cccc(N(c5ccc(-n6c7ccccc7c7ccccc76)cc5)c5cccc6oc7c8ccccc8c(-c8ccccc8)cc7c56)c43)c3ccccc23)cc1 ; c1ccc(-c2cc3c(oc4cccc(N(c5cccc6c5sc5ccccc56)c5cccc6oc7c8ccccc8c(-c8ccccc8)cc7c56)c43)c3ccccc23)cc1 ; c1ccc(-c2cc3c(oc4cccc(N(c5ccc6c(c5)sc5ccccc56)c5cccc6oc7c8ccccc8c(-c8ccccc8)cc7c56)c43)c3ccccc23)cc1]

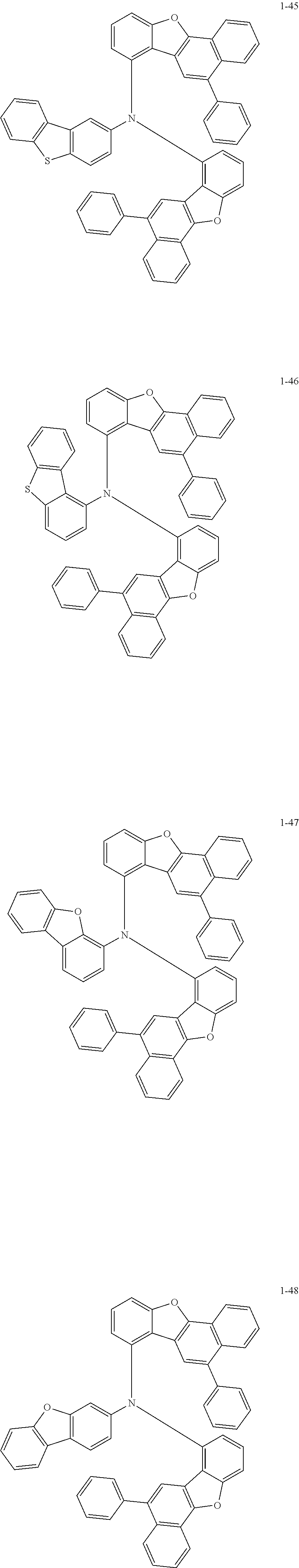 [SMILES: c1ccc(-c2cc3c(oc4cccc(N(c5ccc6sc7ccccc7c6c5)c5cccc6oc7c8ccccc8c(-c8ccccc8)cc7c56)c43)c3ccccc23)cc1 ; c1ccc(-c2cc3c(oc4cccc(N(c5cccc6oc7c8ccccc8c(-c8ccccc8)cc7c56)c5cccc6sc7ccccc7c56)c43)c3ccccc23)cc1 ; c1ccc(-c2cc3c(oc4cccc(N(c5cccc6c5oc5ccccc56)c5cccc6oc7c8ccccc8c(-c8ccccc8)cc7c56)c43)c3ccccc23)cc1 ; c1ccc(-c2cc3c(oc4cccc(N(c5ccc6c(c5)oc5ccccc56)c5cccc6oc7c8ccccc8c(-c8ccccc8)cc7c56)c43)c3ccccc23)cc1]

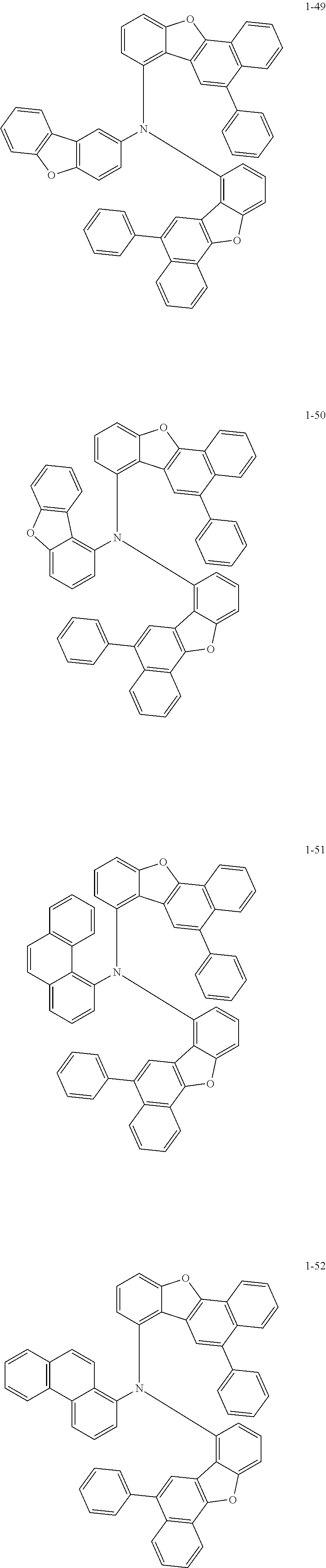 [SMILES: c1ccc(-c2cc3c(oc4cccc(N(c5ccc6oc7ccccc7c6c5)c5cccc6oc7c8ccccc8c(-c8ccccc8)cc7c56)c43)c3ccccc23)cc1 ; c1ccc(-c2cc3c(oc4cccc(N(c5cccc6oc7ccccc7c56)c5cccc6oc7c8ccccc8c(-c8ccccc8)cc7c56)c43)c3ccccc23)cc1 ; c1ccc(-c2cc3c(oc4cccc(N(c5cccc6ccc7ccccc7c56)c5cccc6oc7c8ccccc8c(-c8ccccc8)cc7c56)c43)c3ccccc23)cc1 ; c1ccc(-c2cc3c(oc4cccc(N(c5cccc6c5ccc5ccccc56)c5cccc6oc7c8ccccc8c(-c8ccccc8)cc7c56)c43)c3ccccc23)cc1]

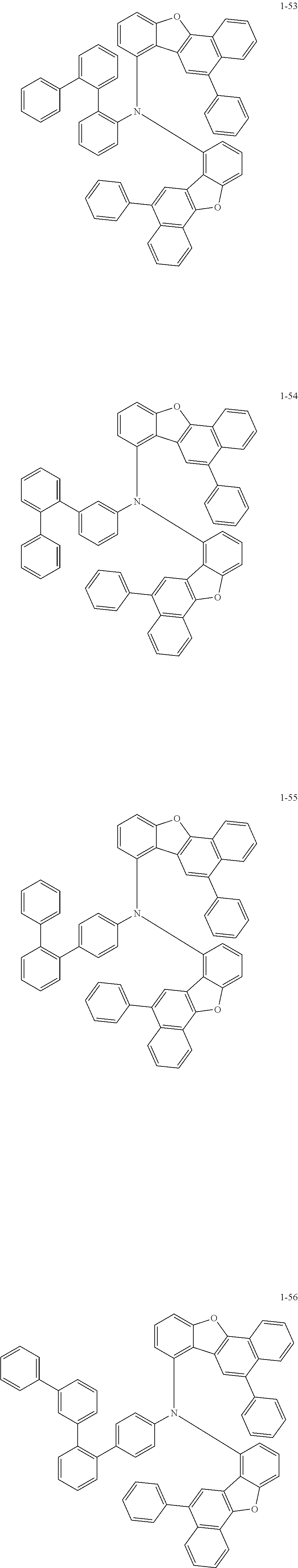 [SMILES: c1ccc(-c2ccccc2-c2ccccc2N(c2cccc3oc4c5ccccc5c(-c5ccccc5)cc4c23)c2cccc3oc4c5ccccc5c(-c5ccccc5)cc4c23)cc1 ; c1ccc(-c2ccccc2-c2cccc(N(c3cccc4oc5c6ccccc6c(-c6ccccc6)cc5c34)c3cccc4oc5c6ccccc6c(-c6ccccc6)cc5c34)c2)cc1 ; c1ccc(-c2ccccc2-c2ccc(N(c3cccc4oc5c6ccccc6c(-c6ccccc6)cc5c34)c3cccc4oc5c6ccccc6c(-c6ccccc6)cc5c34)cc2)cc1 ; c1ccc(-c2cccc(-c3ccccc3-c3ccc(N(c4cccc5oc6c7ccccc7c(-c7ccccc7)cc6c45)c4cccc5oc6c7ccccc7c(-c7ccccc7)cc6c45)cc3)c2)cc1]

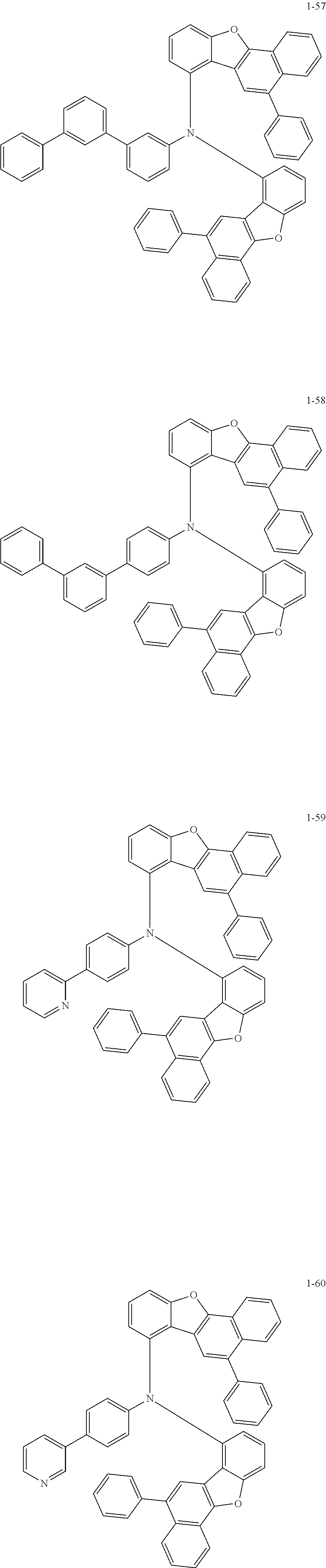 [SMILES: c1ccc(-c2cccc(-c3cccc(N(c4cccc5oc6c7ccccc7c(-c7ccccc7)cc6c45)c4cccc5oc6c7ccccc7c(-c7ccccc7)cc6c45)c3)c2)cc1 ; c1ccc(-c2cccc(-c3ccc(N(c4cccc5oc6c7ccccc7c(-c7ccccc7)cc6c45)c4cccc5oc6c7ccccc7c(-c7ccccc7)cc6c45)cc3)c2)cc1 ; c1ccc(-c2cc3c(oc4cccc(N(c5ccc(-c6ccccn6)cc5)c5cccc6oc7c8ccccc8c(-c8ccccc8)cc7c56)c43)c3ccccc23)cc1 ; c1ccc(-c2cc3c(oc4cccc(N(c5ccc(-c6cccnc6)cc5)c5cccc6oc7c8ccccc8c(-c8ccccc8)cc7c56)c43)c3ccccc23)cc1]

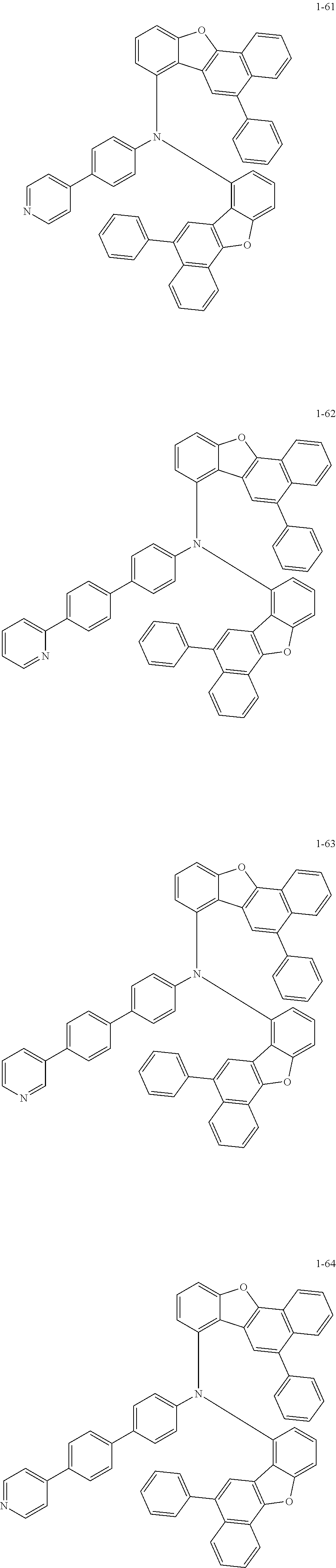 [SMILES: c1ccc(-c2cc3c(oc4cccc(N(c5ccc(-c6ccncc6)cc5)c5cccc6oc7c8ccccc8c(-c8ccccc8)cc7c56)c43)c3ccccc23)cc1 ; c1ccc(-c2cc3c(oc4cccc(N(c5ccc(-c6ccc(-c7ccccn7)cc6)cc5)c5cccc6oc7c8ccccc8c(-c8ccccc8)cc7c56)c43)c3ccccc23)cc1 ; c1ccc(-c2cc3c(oc4cccc(N(c5ccc(-c6ccc(-c7cccnc7)cc6)cc5)c5cccc6oc7c8ccccc8c(-c8ccccc8)cc7c56)c43)c3ccccc23)cc1 ; c1ccc(-c2cc3c(oc4cccc(N(c5ccc(-c6ccc(-c7ccncc7)cc6)cc5)c5cccc6oc7c8ccccc8c(-c8ccccc8)cc7c56)c43)c3ccccc23)cc1]

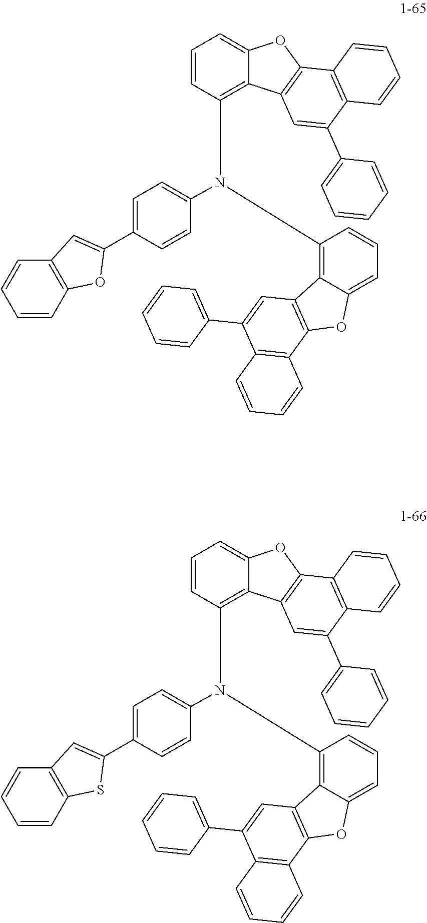 [SMILES: c1ccc(-c2cc3c(oc4cccc(N(c5ccc(-c6cc7ccccc7o6)cc5)c5cccc6oc7c8ccccc8c(-c8ccccc8)cc7c56)c43)c3ccccc23)cc1 ; c1ccc(-c2cc3c(oc4cccc(N(c5ccc(-c6cc7ccccc7s6)cc5)c5cccc6oc7c8ccccc8c(-c8ccccc8)cc7c56)c43)c3ccccc23)cc1]

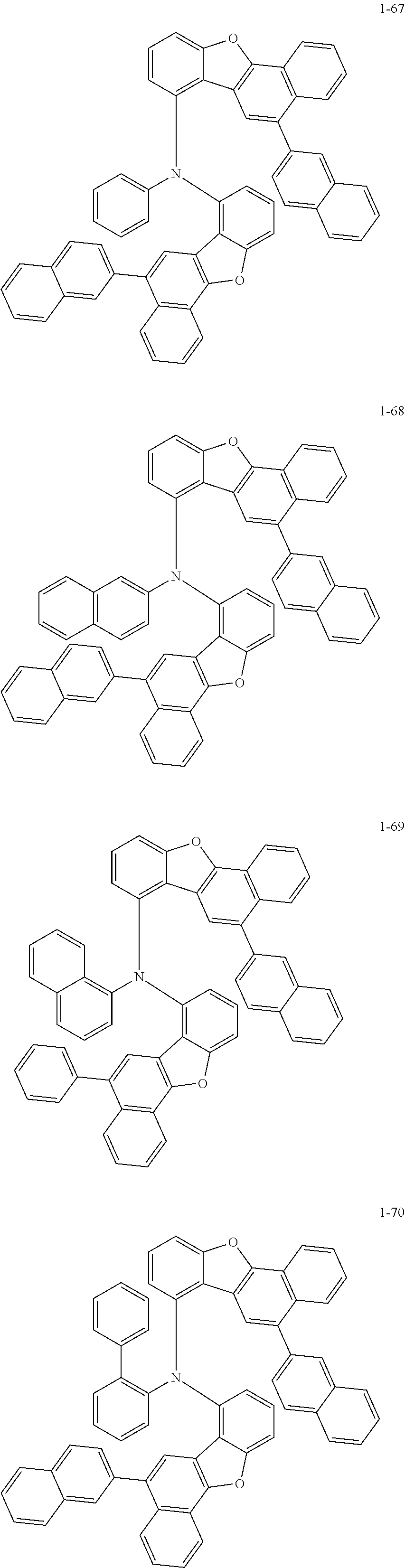 [SMILES: c1ccc(N(c2cccc3oc4c5ccccc5c(-c5ccc6ccccc6c5)cc4c23)c2cccc3oc4c5ccccc5c(-c5ccc6ccccc6c5)cc4c23)cc1 ; c1ccc2cc(-c3cc4c(oc5cccc(N(c6ccc7ccccc7c6)c6cccc7oc8c9ccccc9c(-c9ccc%10ccccc%10c9)cc8c67)c54)c4ccccc34)ccc2c1 ; c1ccc(-c2cc3c(oc4cccc(N(c5cccc6ccccc56)c5cccc6oc7c8ccccc8c(-c8ccc9ccccc9c8)cc7c56)c43)c3ccccc23)cc1 ; c1ccc(-c2ccccc2N(c2cccc3oc4c5ccccc5c(-c5ccc6ccccc6c5)cc4c23)c2cccc3oc4c5ccccc5c(-c5ccc6ccccc6c5)cc4c23)cc1]

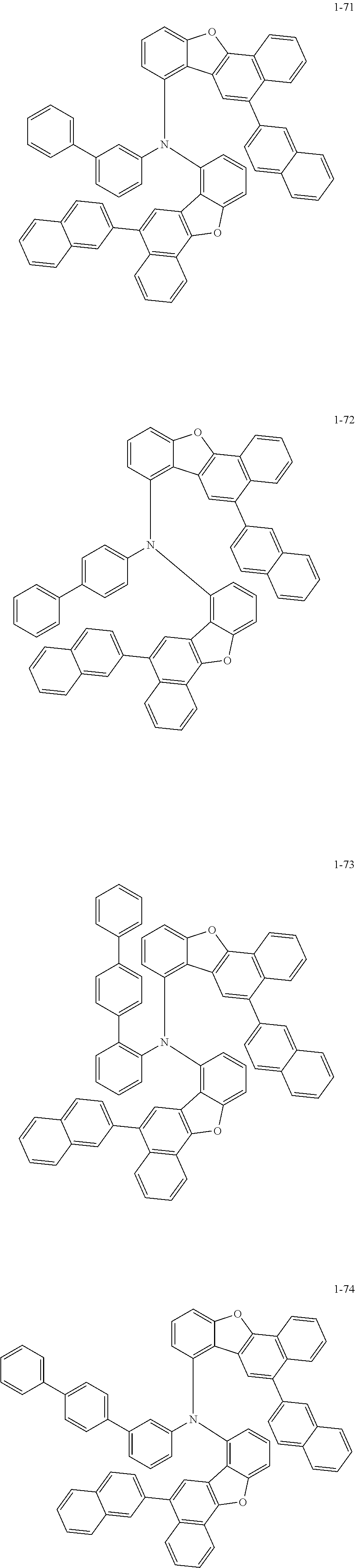 [SMILES: c1ccc(-c2cccc(N(c3cccc4oc5c6ccccc6c(-c6ccc7ccccc7c6)cc5c34)c3cccc4oc5c6ccccc6c(-c6ccc7ccccc7c6)cc5c34)c2)cc1 ; c1ccc(-c2ccc(N(c3cccc4oc5c6ccccc6c(-c6ccc7ccccc7c6)cc5c34)c3cccc4oc5c6ccccc6c(-c6ccc7ccccc7c6)cc5c34)cc2)cc1 ; c1ccc(-c2ccc(-c3ccccc3N(c3cccc4oc5c6ccccc6c(-c6ccc7ccccc7c6)cc5c34)c3cccc4oc5c6ccccc6c(-c6ccc7ccccc7c6)cc5c34)cc2)cc1 ; c1ccc(-c2ccc(-c3cccc(N(c4cccc5oc6c7ccccc7c(-c7ccc8ccccc8c7)cc6c45)c4cccc5oc6c7ccccc7c(-c7ccc8ccccc8c7)cc6c45)c3)cc2)cc1]

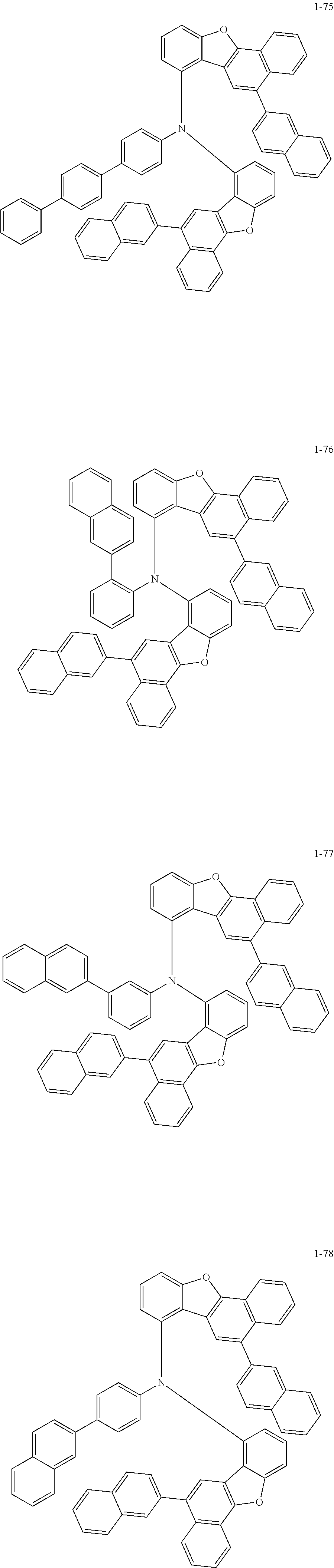 [SMILES: c1ccc(-c2ccc(-c3ccc(N(c4cccc5oc6c7ccccc7c(-c7ccc8ccccc8c7)cc6c45)c4cccc5oc6c7ccccc7c(-c7ccc8ccccc8c7)cc6c45)cc3)cc2)cc1 ; c1ccc(N(c2cccc3oc4c5ccccc5c(-c5ccc6ccccc6c5)cc4c23)c2cccc3oc4c5ccccc5c(-c5ccc6ccccc6c5)cc4c23)c(-c2ccc3ccccc3c2)c1 ; c1cc(-c2ccc3ccccc3c2)cc(N(c2cccc3oc4c5ccccc5c(-c5ccc6ccccc6c5)cc4c23)c2cccc3oc4c5ccccc5c(-c5ccc6ccccc6c5)cc4c23)c1 ; c1ccc2cc(-c3ccc(N(c4cccc5oc6c7ccccc7c(-c7ccc8ccccc8c7)cc6c45)c4cccc5oc6c7ccccc7c(-c7ccc8ccccc8c7)cc6c45)cc3)ccc2c1]

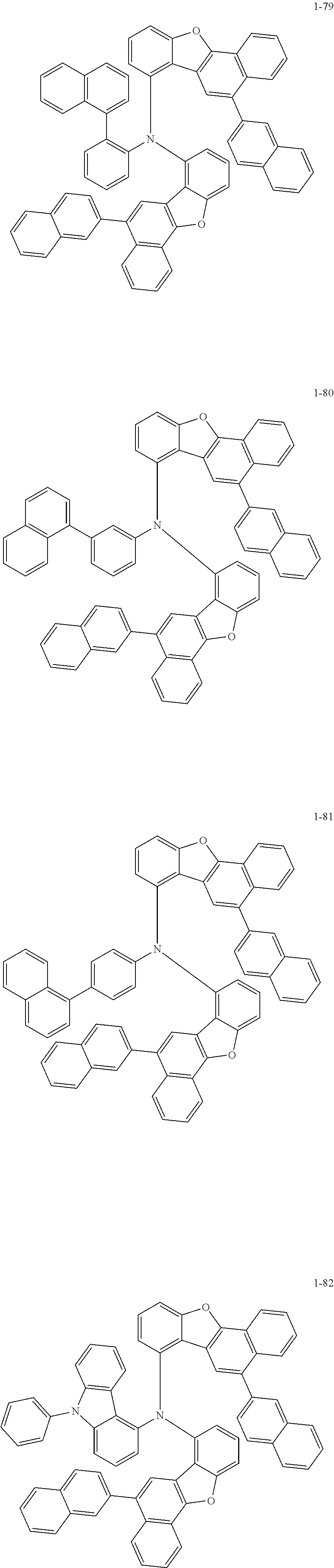 [SMILES: c1ccc(N(c2cccc3oc4c5ccccc5c(-c5ccc6ccccc6c5)cc4c23)c2cccc3oc4c5ccccc5c(-c5ccc6ccccc6c5)cc4c23)c(-c2cccc3ccccc23)c1 ; c1cc(-c2cccc3ccccc23)cc(N(c2cccc3oc4c5ccccc5c(-c5ccc6ccccc6c5)cc4c23)c2cccc3oc4c5ccccc5c(-c5ccc6ccccc6c5)cc4c23)c1 ; c1ccc2cc(-c3cc4c(oc5cccc(N(c6ccc(-c7cccc8ccccc78)cc6)c6cccc7oc8c9ccccc9c(-c9ccc%10ccccc%10c9)cc8c67)c54)c4ccccc34)ccc2c1 ; c1ccc(-n2c3ccccc3c3c(N(c4cccc5oc6c7ccccc7c(-c7ccc8ccccc8c7)cc6c45)c4cccc5oc6c7ccccc7c(-c7ccc8ccccc8c7)cc6c45)cccc32)cc1]

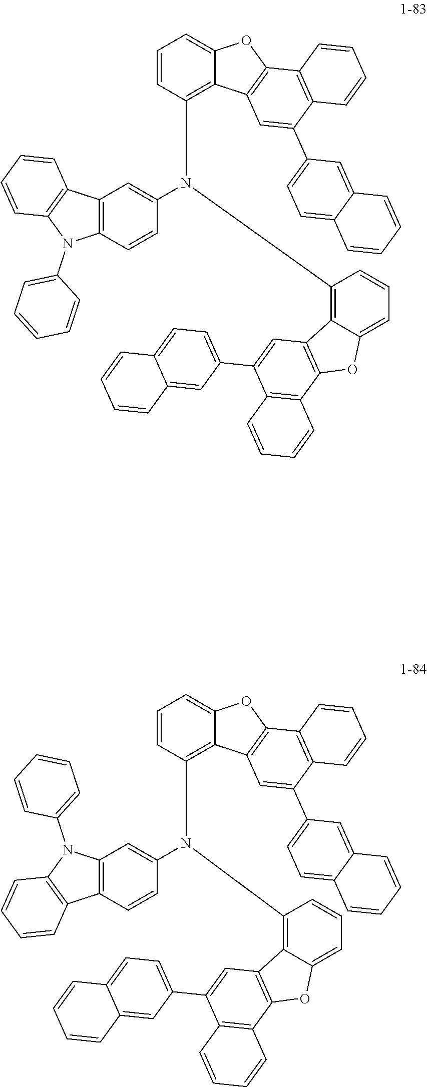 [SMILES: c1ccc(-n2c3ccccc3c3cc(N(c4cccc5oc6c7ccccc7c(-c7ccc8ccccc8c7)cc6c45)c4cccc5oc6c7ccccc7c(-c7ccc8ccccc8c7)cc6c45)ccc32)cc1 ; c1ccc(-n2c3ccccc3c3ccc(N(c4cccc5oc6c7ccccc7c(-c7ccc8ccccc8c7)cc6c45)c4cccc5oc6c7ccccc7c(-c7ccc8ccccc8c7)cc6c45)cc32)cc1]

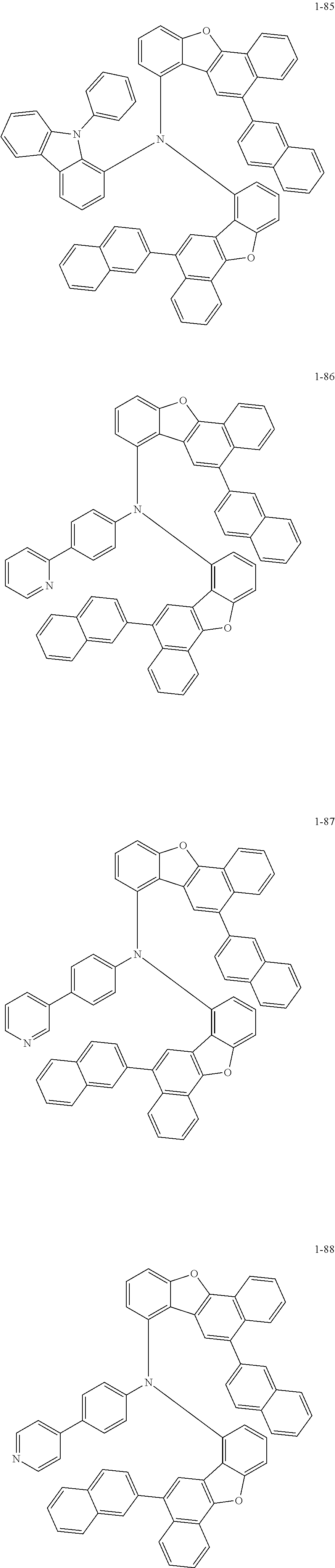 [SMILES: c1ccc(-n2c3ccccc3c3cccc(N(c4cccc5oc6c7ccccc7c(-c7ccc8ccccc8c7)cc6c45)c4cccc5oc6c7ccccc7c(-c7ccc8ccccc8c7)cc6c45)c32)cc1 ; c1ccc(-c2ccc(N(c3cccc4oc5c6ccccc6c(-c6ccc7ccccc7c6)cc5c34)c3cccc4oc5c6ccccc6c(-c6ccc7ccccc7c6)cc5c34)cc2)nc1 ; c1cncc(-c2ccc(N(c3cccc4oc5c6ccccc6c(-c6ccc7ccccc7c6)cc5c34)c3cccc4oc5c6ccccc6c(-c6ccc7ccccc7c6)cc5c34)cc2)c1 ; c1ccc2cc(-c3cc4c(oc5cccc(N(c6ccc(-c7ccncc7)cc6)c6cccc7oc8c9ccccc9c(-c9ccc%10ccccc%10c9)cc8c67)c54)c4ccccc34)ccc2c1]

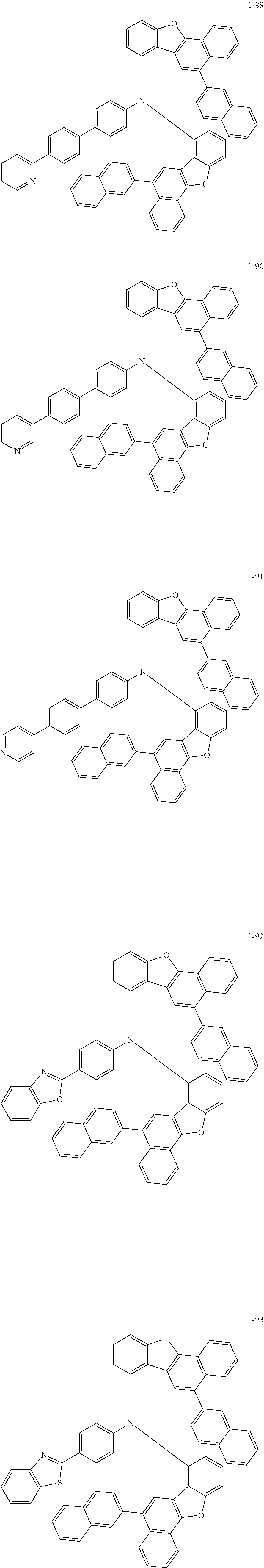

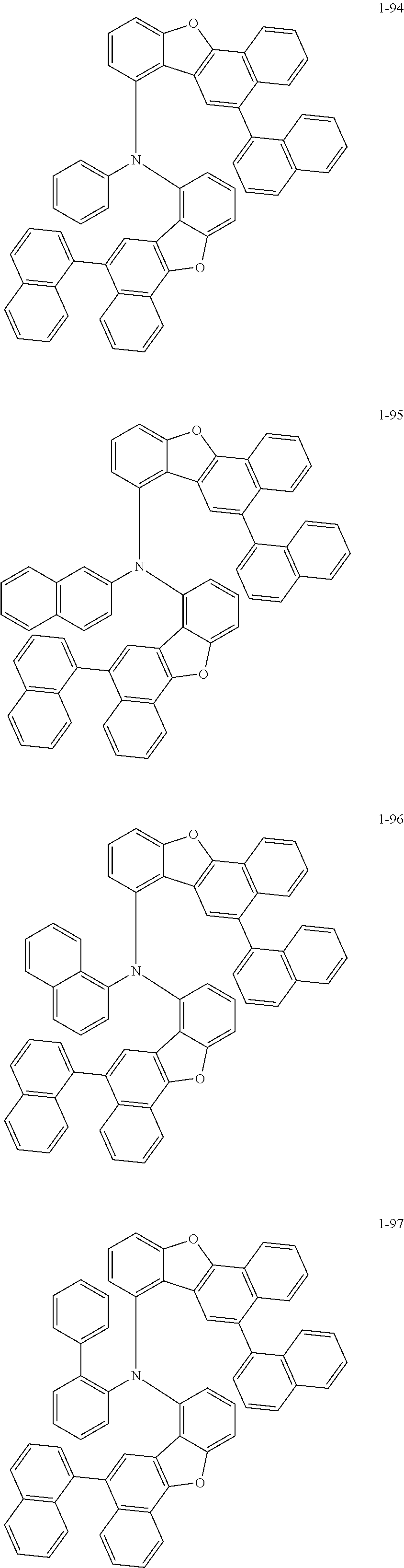 [SMILES: c1ccc(N(c2cccc3oc4c5ccccc5c(-c5cccc6ccccc56)cc4c23)c2cccc3oc4c5ccccc5c(-c5cccc6ccccc56)cc4c23)cc1 ; c1ccc2cc(N(c3cccc4oc5c6ccccc6c(-c6cccc7ccccc67)cc5c34)c3cccc4oc5c6ccccc6c(-c6cccc7ccccc67)cc5c34)ccc2c1 ; c1ccc2c(-c3cc4c(oc5cccc(N(c6cccc7ccccc67)c6cccc7oc8c9ccccc9c(-c9cccc%10ccccc9%10)cc8c67)c54)c4ccccc34)cccc2c1 ; c1ccc(-c2ccccc2N(c2cccc3oc4c5ccccc5c(-c5cccc6ccccc56)cc4c23)c2cccc3oc4c5ccccc5c(-c5cccc6ccccc56)cc4c23)cc1]

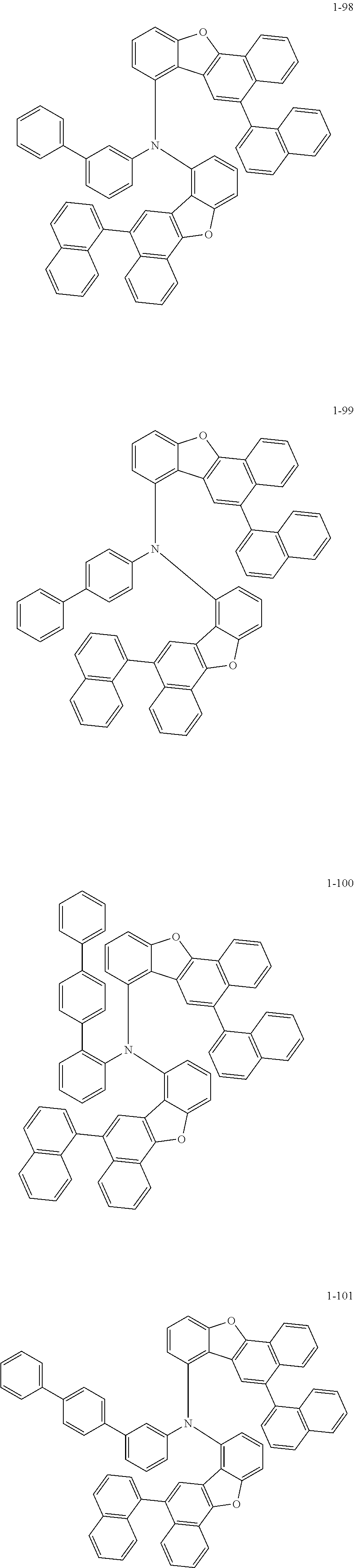 [SMILES: c1ccc(-c2cccc(N(c3cccc4oc5c6ccccc6c(-c6cccc7ccccc67)cc5c34)c3cccc4oc5c6ccccc6c(-c6cccc7ccccc67)cc5c34)c2)cc1 ; c1ccc(-c2ccc(N(c3cccc4oc5c6ccccc6c(-c6cccc7ccccc67)cc5c34)c3cccc4oc5c6ccccc6c(-c6cccc7ccccc67)cc5c34)cc2)cc1 ; c1ccc(-c2ccc(-c3ccccc3N(c3cccc4oc5c6ccccc6c(-c6cccc7ccccc67)cc5c34)c3cccc4oc5c6ccccc6c(-c6cccc7ccccc67)cc5c34)cc2)cc1 ; c1ccc(-c2ccc(-c3cccc(N(c4cccc5oc6c7ccccc7c(-c7cccc8ccccc78)cc6c45)c4cccc5oc6c7ccccc7c(-c7cccc8ccccc78)cc6c45)c3)cc2)cc1]

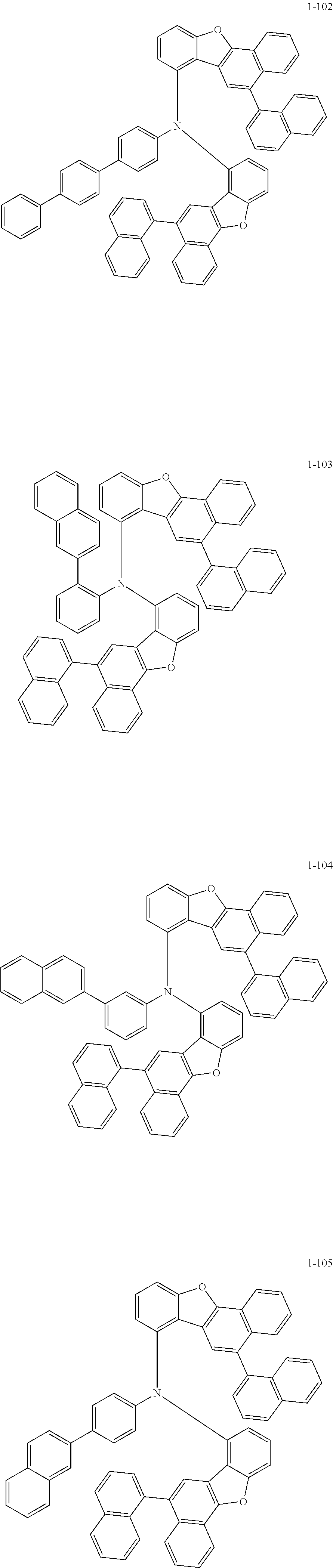 [SMILES: c1ccc(-c2ccc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2)cc1 ; c1ccc(N(c2cccc3oc4c5ccccc5c(-c5cccc6ccccc56)cc4c23)c2cccc3oc4c5ccccc5c(-c5cccc6ccccc56)cc4c23)c(-c2ccc3ccccc3c2)c1 ; c1cc(-c2ccc3ccccc3c2)cc(N(c2cccc3oc4c5ccccc5c(-c5cccc6ccccc56)cc4c23)c2cccc3oc4c5ccccc5c(-c5cccc6ccccc56)cc4c23)c1 ; c1ccc2cc(-c3ccc(N(c4cccc5oc6c7ccccc7c(-c7cccc8ccccc78)cc6c45)c4cccc5oc6c7ccccc7c(-c7cccc8ccccc78)cc6c45)cc3)ccc2c1]

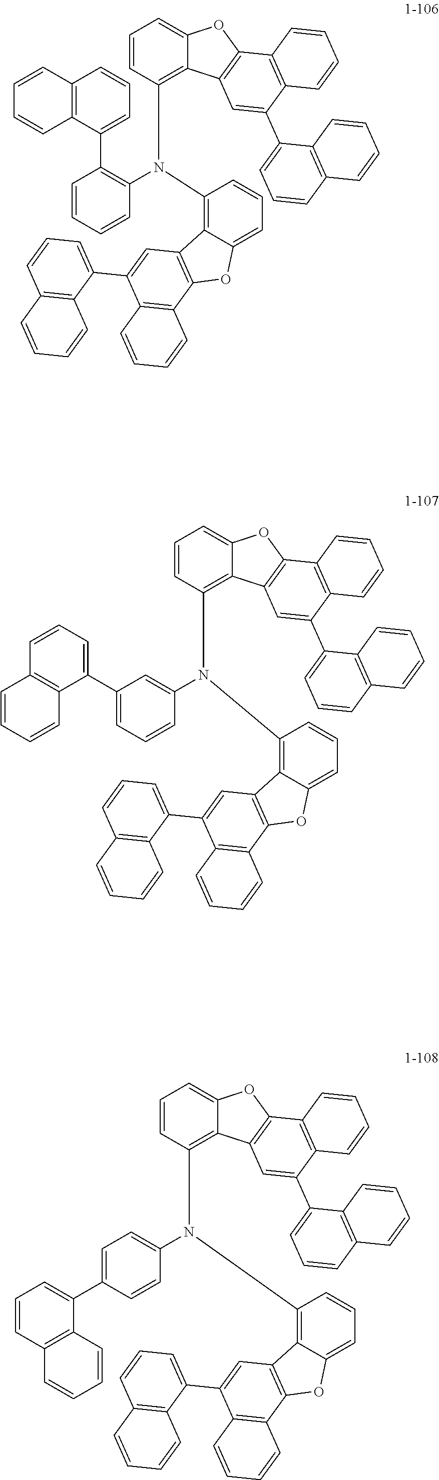 [SMILES: c1ccc(N(c2cccc3oc4c5ccccc5c(-c5cccc6ccccc56)cc4c23)c2cccc3oc4c5ccccc5c(-c5cccc6ccccc56)cc4c23)c(-c2cccc3ccccc23)c1 ; c1cc(-c2cccc3ccccc23)cc(N(c2cccc3oc4c5ccccc5c(-c5cccc6ccccc56)cc4c23)c2cccc3oc4c5ccccc5c(-c5cccc6ccccc56)cc4c23)c1 ; c1ccc2c(-c3ccc(N(c4cccc5oc6c7ccccc7c(-c7cccc8ccccc78)cc6c45)c4cccc5oc6c7ccccc7c(-c7cccc8ccccc78)cc6c45)cc3)cccc2c1]

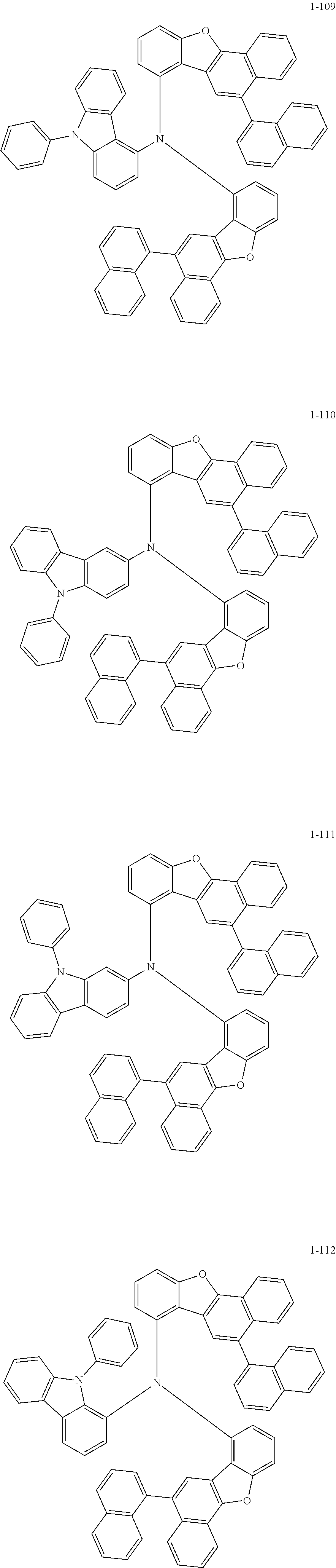 [SMILES: c1ccc(-n2c3ccccc3c3c(N(c4cccc5oc6c7ccccc7c(-c7cccc8ccccc78)cc6c45)c4cccc5oc6c7ccccc7c(-c7cccc8ccccc78)cc6c45)cccc32)cc1 ; c1ccc(-n2c3ccccc3c3cc(N(c4cccc5oc6c7ccccc7c(-c7cccc8ccccc78)cc6c45)c4cccc5oc6c7ccccc7c(-c7cccc8ccccc78)cc6c45)ccc32)cc1 ; c1ccc(-n2c3ccccc3c3ccc(N(c4cccc5oc6c7ccccc7c(-c7cccc8ccccc78)cc6c45)c4cccc5oc6c7ccccc7c(-c7cccc8ccccc78)cc6c45)cc32)cc1 ; c1ccc(-n2c3ccccc3c3cccc(N(c4cccc5oc6c7ccccc7c(-c7cccc8ccccc78)cc6c45)c4cccc5oc6c7ccccc7c(-c7cccc8ccccc78)cc6c45)c32)cc1]

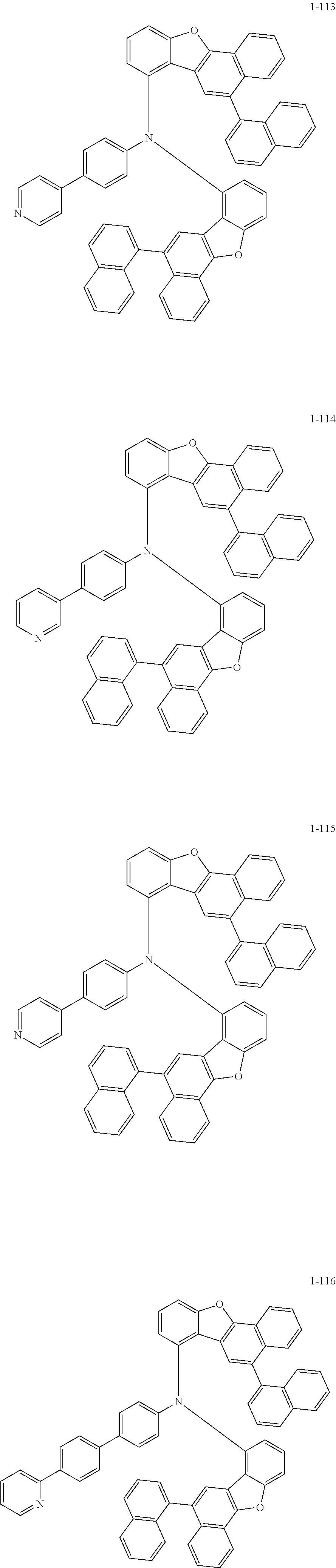 [SMILES: c1ccc2c(-c3cc4c(oc5cccc(N(c6ccc(-c7ccncc7)cc6)c6cccc7oc8c9ccccc9c(-c9cccc%10ccccc9%10)cc8c67)c54)c4ccccc34)cccc2c1 ; c1cncc(-c2ccc(N(c3cccc4oc5c6ccccc6c(-c6cccc7ccccc67)cc5c34)c3cccc4oc5c6ccccc6c(-c6cccc7ccccc67)cc5c34)cc2)c1 ; c1ccc2c(-c3cc4c(oc5cccc(N(c6ccc(-c7ccncc7)cc6)c6cccc7oc8c9ccccc9c(-c9cccc%10ccccc9%10)cc8c67)c54)c4ccccc34)cccc2c1 ; c1ccc(-c2ccc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2)nc1]

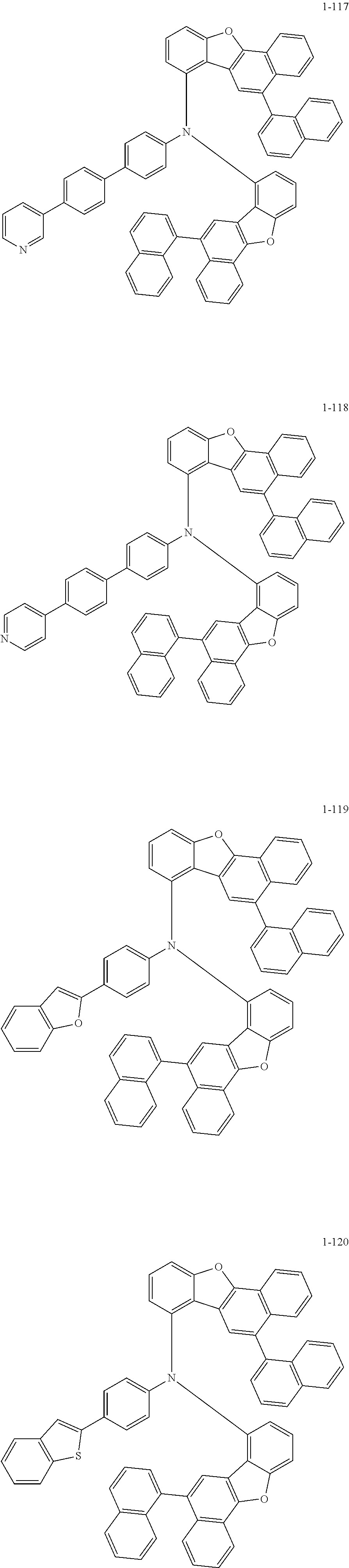 [SMILES: c1cncc(-c2ccc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2)c1 ; c1ccc2c(-c3cc4c(oc5cccc(N(c6ccc(-c7ccc(-c8ccncc8)cc7)cc6)c6cccc7oc8c9ccccc9c(-c9cccc%10ccccc9%10)cc8c67)c54)c4ccccc34)cccc2c1 ; c1ccc2oc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2c1 ; c1ccc2sc(-c3ccc(N(c4cccc5oc6c7ccccc7c(-c7cccc8ccccc78)cc6c45)c4cccc5oc6c7ccccc7c(-c7cccc8ccccc78)cc6c45)cc3)cc2c1]

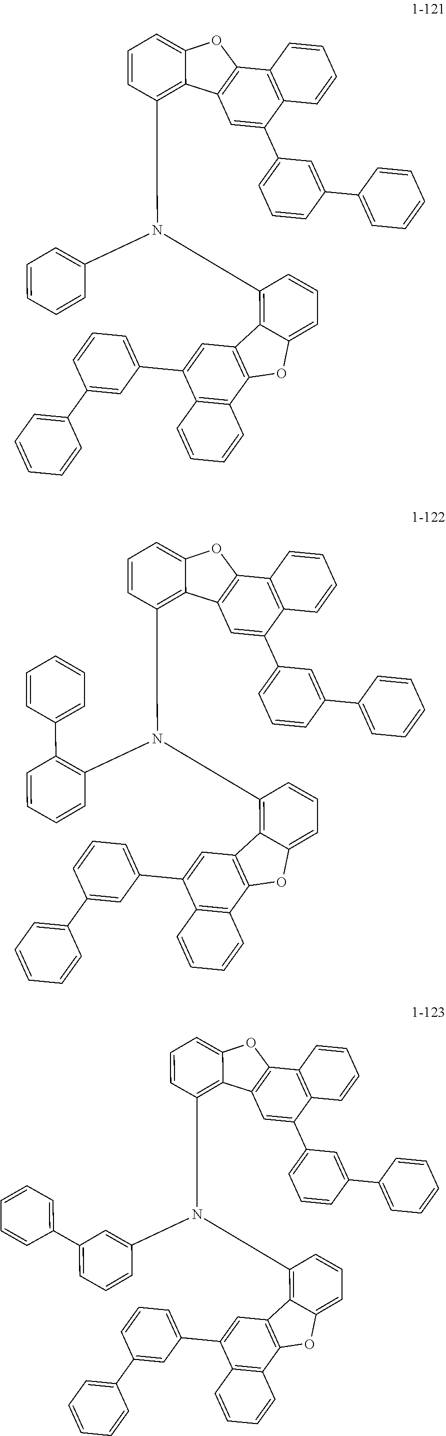 [SMILES: c1ccc(-c2cccc(-c3cc4c(oc5cccc(N(c6ccccc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccccc6-c6ccccc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc(-c7ccccc7)c6)c6cccc7oc8c9ccccc9c(-c9cccc(-c%10ccccc%10)c9)cc8c67)c54)c4ccccc34)c2)cc1]

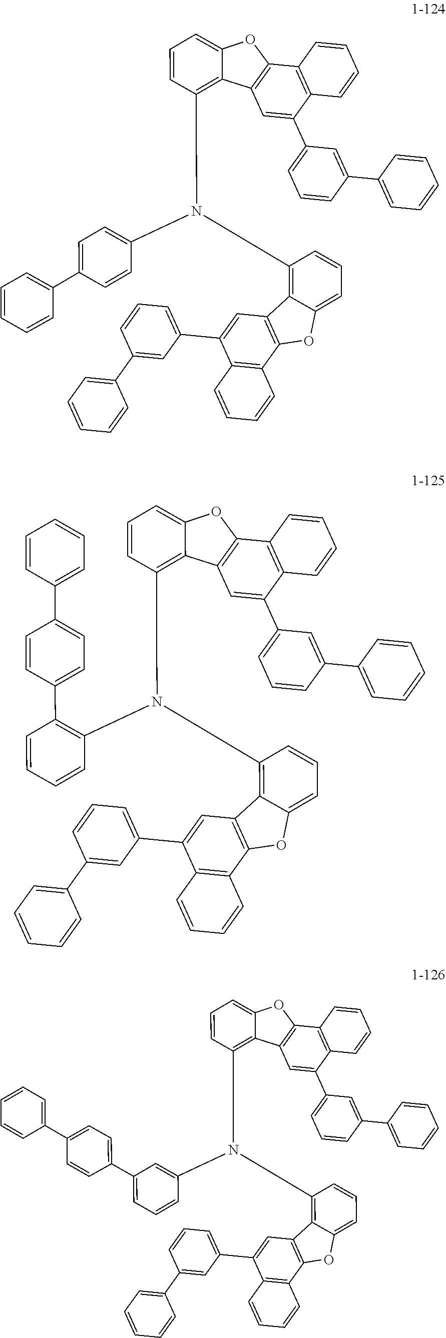 [SMILES: c1ccc(-c2ccc(N(c3cccc4oc5c6ccccc6c(-c6cccc(-c7ccccc7)c6)cc5c34)c3cccc4oc5c6ccccc6c(-c6cccc(-c7ccccc7)c6)cc5c34)cc2)cc1 ; c1ccc(-c2ccc(-c3ccccc3N(c3cccc4oc5c6ccccc6c(-c6cccc(-c7ccccc7)c6)cc5c34)c3cccc4oc5c6ccccc6c(-c6cccc(-c7ccccc7)c6)cc5c34)cc2)cc1 ; c1ccc(-c2ccc(-c3cccc(N(c4cccc5oc6c7ccccc7c(-c7cccc(-c8ccccc8)c7)cc6c45)c4cccc5oc6c7ccccc7c(-c7cccc(-c8ccccc8)c7)cc6c45)c3)cc2)cc1]

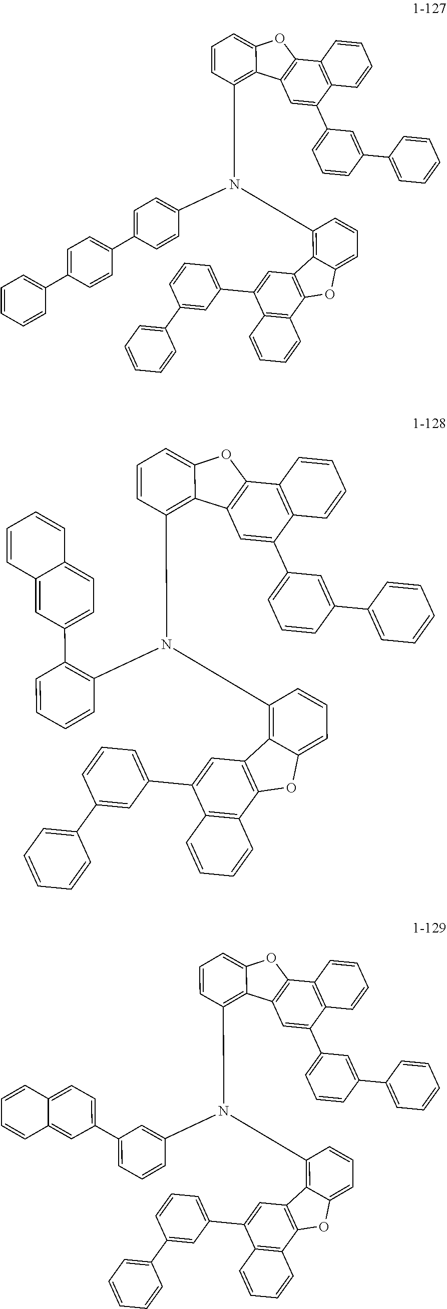 [SMILES: c1ccc(-c2ccc(-c3ccc(N(c4cccc5oc6c7ccccc7c(-c7cccc(-c8ccccc8)c7)cc6c45)c4cccc5oc6c7ccccc7c(-c7cccc(-c8ccccc8)c7)cc6c45)cc3)cc2)cc1 ; c1ccc(-c2cccc(-c3cc4c(oc5cccc(N(c6ccccc6-c6ccc7ccccc7c6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc(-c7ccc8ccccc8c7)c6)c6cccc7oc8c9ccccc9c(-c9cccc(-c%10ccccc%10)c9)cc8c67)c54)c4ccccc34)c2)cc1]

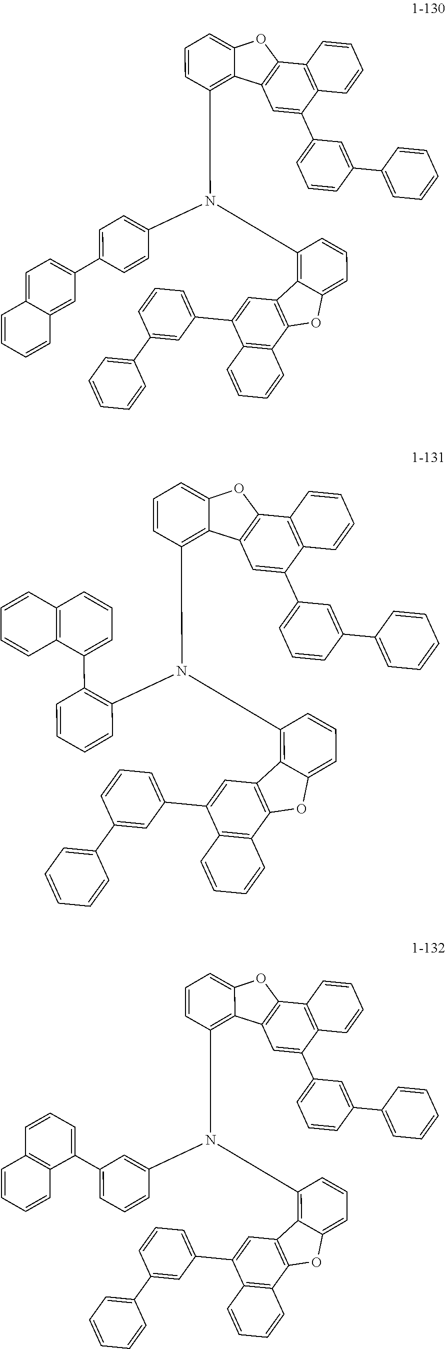 [SMILES: c1ccc(-c2cccc(-c3cc4c(oc5cccc(N(c6ccc(-c7ccc8ccccc8c7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccccc6-c6cccc7ccccc67)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc(-c7cccc8ccccc78)c6)c6cccc7oc8c9ccccc9c(-c9cccc(-c%10ccccc%10)c9)cc8c67)c54)c4ccccc34)c2)cc1]

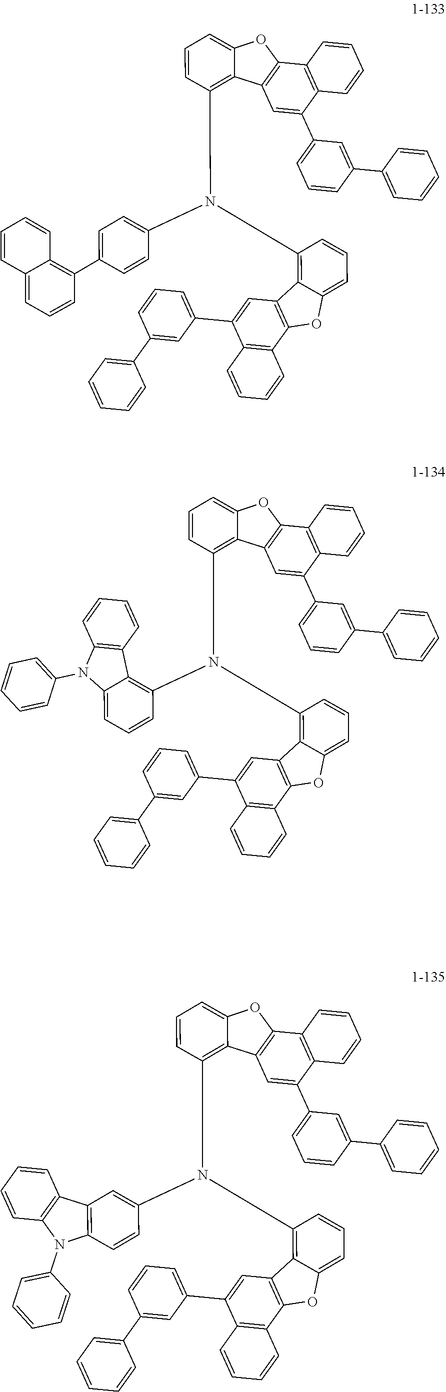 [SMILES: c1ccc(-c2cccc(-c3cc4c(oc5cccc(N(c6ccc(-c7cccc8ccccc78)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc7oc8c9ccccc9c(-c9cccc(-c%10ccccc%10)c9)cc8c67)c6cccc7c6c6ccccc6n7-c6ccccc6)c54)c4ccccc34)c2)cc1 ; c1ccc(-c2cccc(-c3cc4c(oc5cccc(N(c6ccc7c(c6)c6ccccc6n7-c6ccccc6)c6cccc7oc8c9ccccc9c(-c9cccc(-c%10ccccc%10)c9)cc8c67)c54)c4ccccc34)c2)cc1]

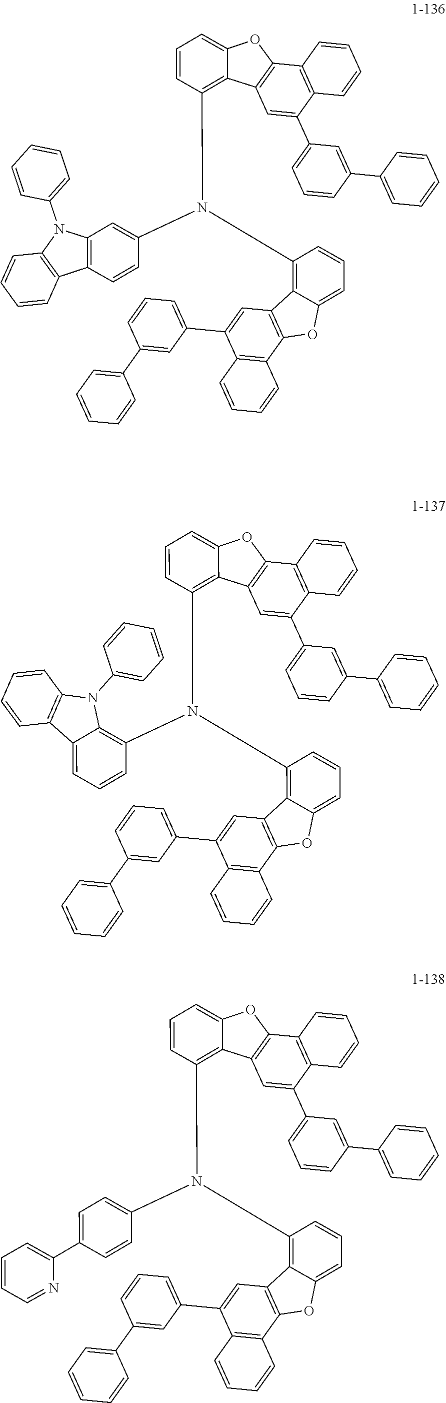 [SMILES: c1ccc(-c2cccc(-c3cc4c(oc5cccc(N(c6ccc7c8ccccc8n(-c8ccccc8)c7c6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6cccc7oc8c9ccccc9c(-c9cccc(-c%10ccccc%10)c9)cc8c67)c6cccc7c8ccccc8n(-c8ccccc8)c67)c54)c4ccccc34)c2)cc1 ; c1ccc(-c2cccc(-c3cc4c(oc5cccc(N(c6ccc(-c7ccccn7)cc6)c6cccc7oc8c9ccccc9c(-c9cccc(-c%10ccccc%10)c9)cc8c67)c54)c4ccccc34)c2)cc1]

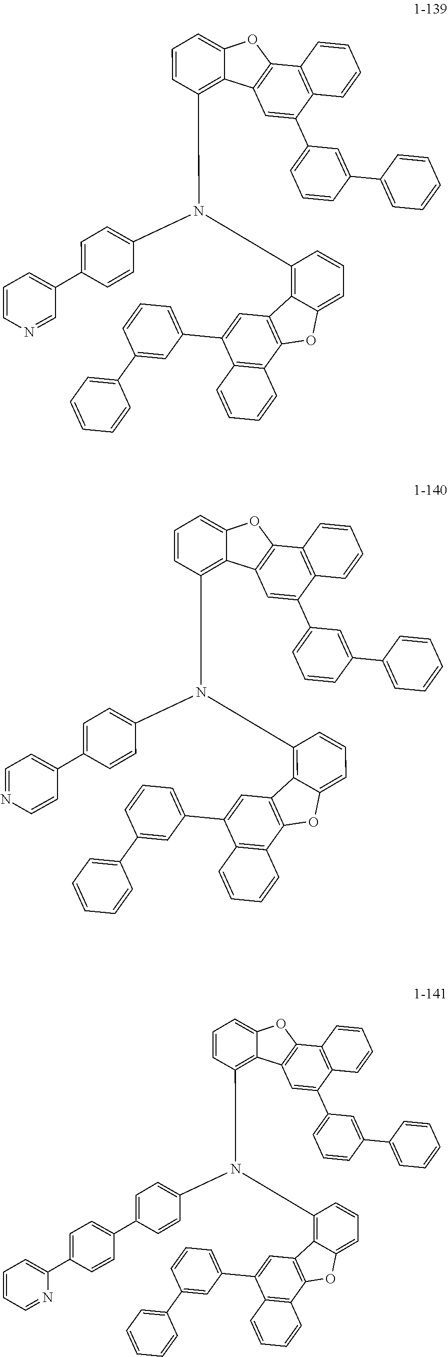 [SMILES: c1ccc(-c2cccc(-c3cc4c(oc5cccc(N(c6ccc(-c7cccnc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7ccncc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7ccc(-c8ccccn8)cc7)cc6)c6cccc7oc8c9ccccc9c(-c9cccc(-c%10ccccc%10)c9)cc8c67)c54)c4ccccc34)c2)cc1]

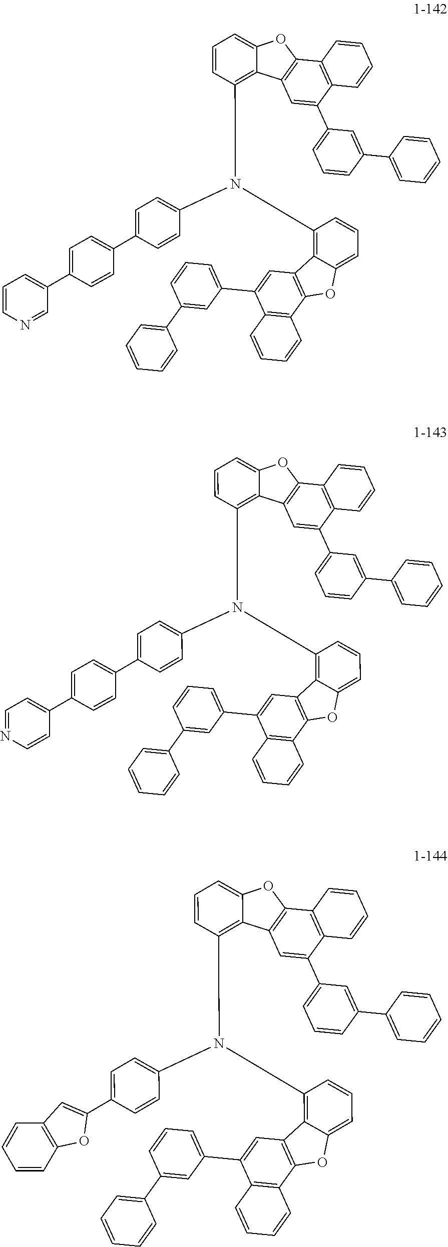 [SMILES: c1ccc(-c2cccc(-c3cc4c(oc5cccc(N(c6ccc(-c7ccc(-c8cccnc8)cc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7ccc(-c8ccncc8)cc7)cc6)c6cccc7oc8c9ccccc9c(-c9cccc(-c%10ccccc%10)c9)cc8c67)c54)c4ccccc34)c2)cc1 ; c1ccc(-c2cccc(-c3cc4c(oc5cccc(N(c6ccc(-c7cc8ccccc8o7)cc6)c6cccc7oc8c9ccccc9c(-c9cccc(-c%10ccccc%10)c9)cc8c67)c54)c4ccccc34)c2)cc1]

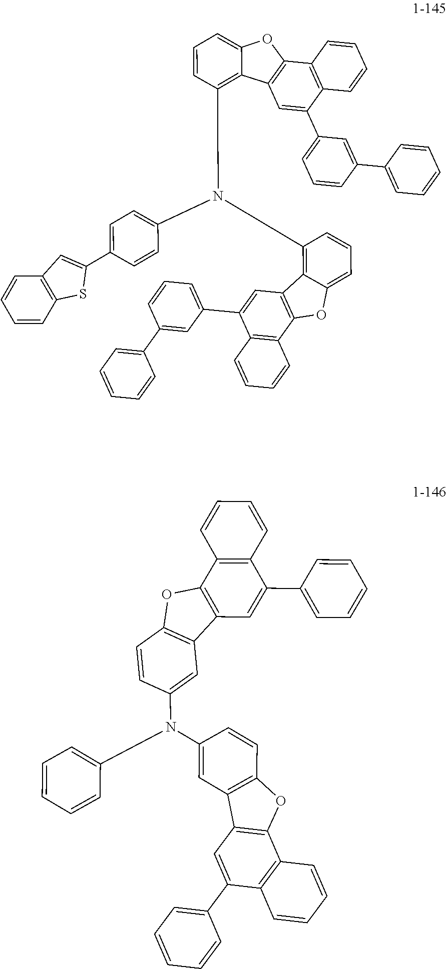 [SMILES: c1ccc(-c2cccc(-c3cc4c(oc5cccc(N(c6ccc(-c7cc8ccccc8s7)cc6)c6cccc7oc8c9ccccc9c(-c9cccc(-c%10ccccc%10)c9)cc8c67)c54)c4ccccc34)c2)cc1 ; c1ccc(-c2cc3c4cc(N(c5ccccc5)c5ccc6oc7c8ccccc8c(-c8ccccc8)cc7c6c5)ccc4oc3c3ccccc23)cc1]

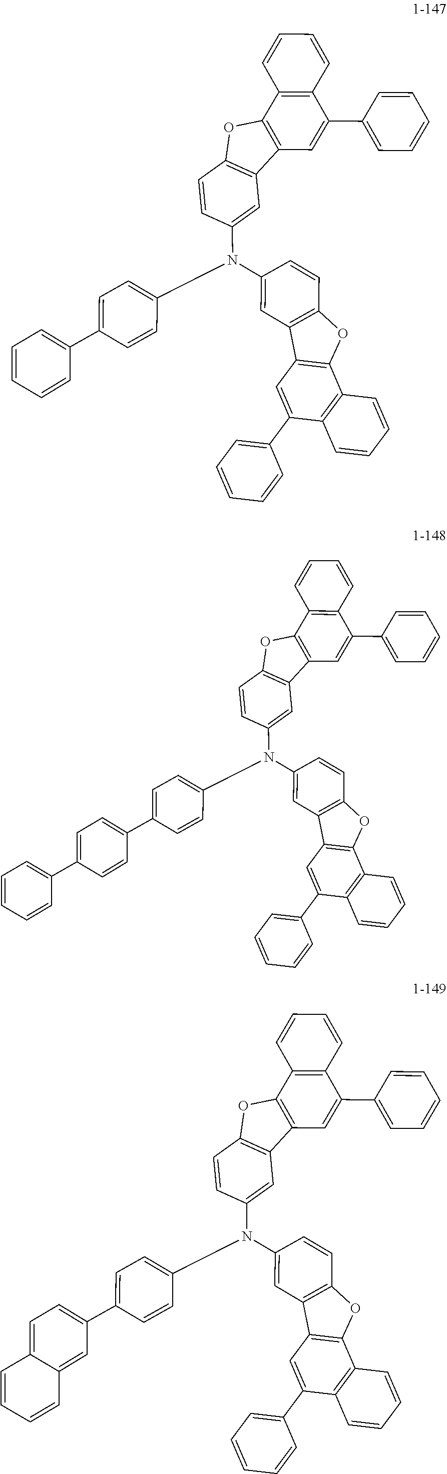 [SMILES: c1ccc(-c2ccc(N(c3ccc4oc5c6ccccc6c(-c6ccccc6)cc5c4c3)c3ccc4oc5c6ccccc6c(-c6ccccc6)cc5c4c3)cc2)cc1 ; c1ccc(-c2ccc(-c3ccc(N(c4ccc5oc6c7ccccc7c(-c7ccccc7)cc6c5c4)c4ccc5oc6c7ccccc7c(-c7ccccc7)cc6c5c4)cc3)cc2)cc1 ; c1ccc(-c2cc3c4cc(N(c5ccc(-c6ccc7ccccc7c6)cc5)c5ccc6oc7c8ccccc8c(-c8ccccc8)cc7c6c5)ccc4oc3c3ccccc23)cc1]

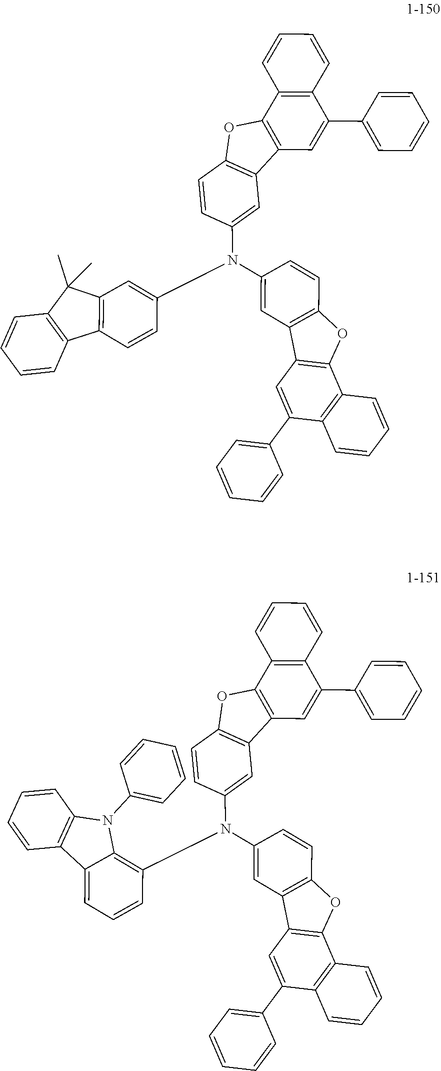 [SMILES: CC1(C)c2ccccc2-c2ccc(N(c3ccc4oc5c6ccccc6c(-c6ccccc6)cc5c4c3)c3ccc4oc5c6ccccc6c(-c6ccccc6)cc5c4c3)cc21 ; c1ccc(-c2cc3c4cc(N(c5ccc6oc7c8ccccc8c(-c8ccccc8)cc7c6c5)c5cccc6c7ccccc7n(-c7ccccc7)c56)ccc4oc3c3ccccc23)cc1]

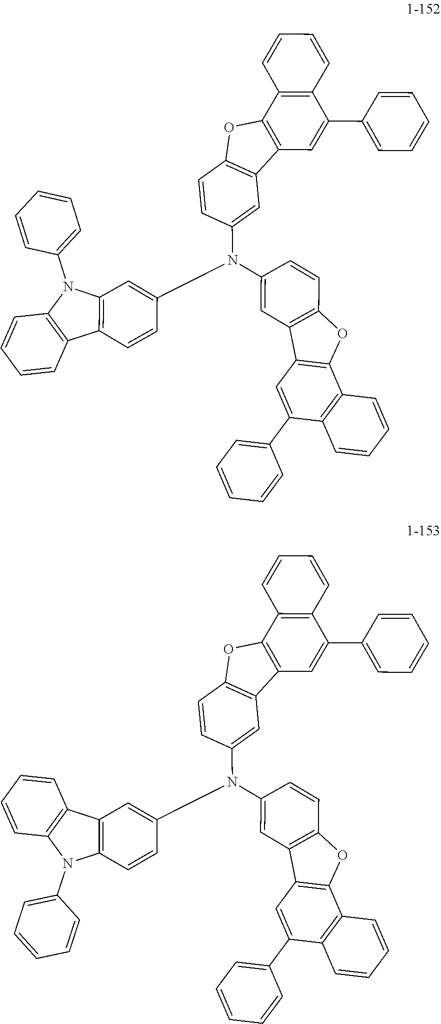 [SMILES: c1ccc(-c2cc3c4cc(N(c5ccc6oc7c8ccccc8c(-c8ccccc8)cc7c6c5)c5ccc6c7ccccc7n(-c7ccccc7)c6c5)ccc4oc3c3ccccc23)cc1 ; c1ccc(-c2cc3c4cc(N(c5ccc6oc7c8ccccc8c(-c8ccccc8)cc7c6c5)c5ccc6c(c5)c5ccccc5n6-c5ccccc5)ccc4oc3c3ccccc23)cc1]

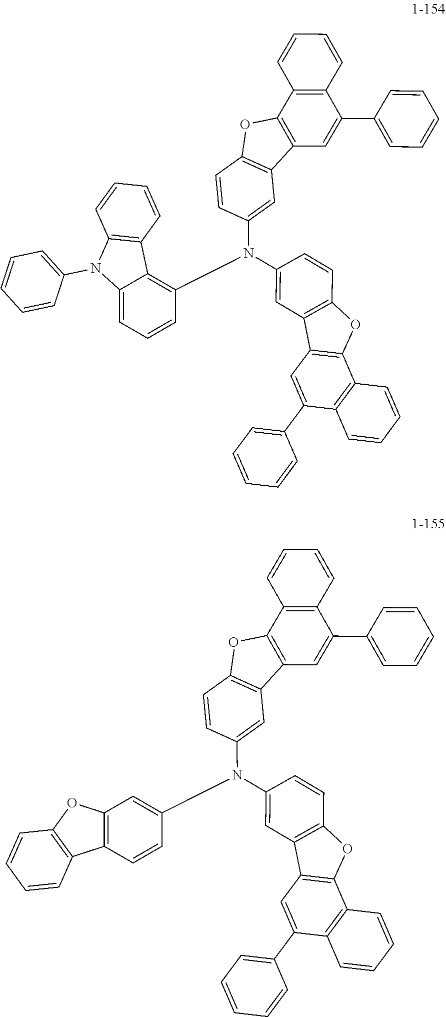 [SMILES: c1ccc(-c2cc3c4cc(N(c5ccc6oc7c8ccccc8c(-c8ccccc8)cc7c6c5)c5cccc6c5c5ccccc5n6-c5ccccc5)ccc4oc3c3ccccc23)cc1 ; c1ccc(-c2cc3c4cc(N(c5ccc6c(c5)oc5ccccc56)c5ccc6oc7c8ccccc8c(-c8ccccc8)cc7c6c5)ccc4oc3c3ccccc23)cc1]

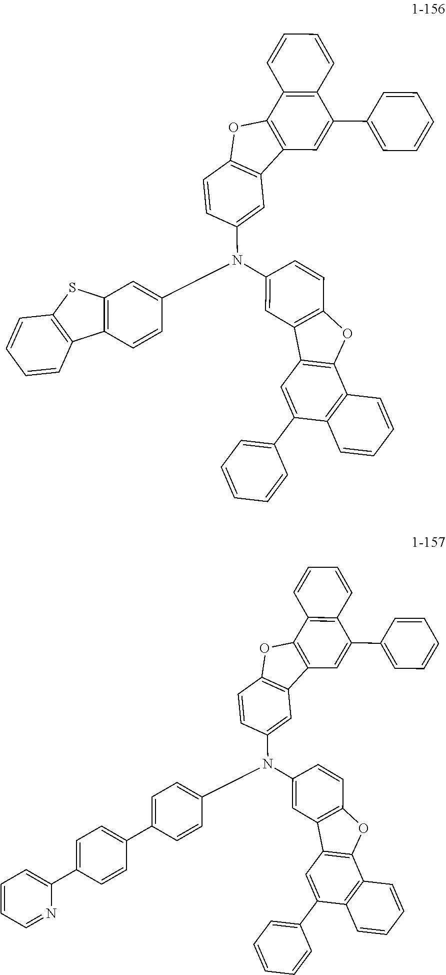 [SMILES: c1ccc(-c2cc3c4cc(N(c5ccc6c(c5)sc5ccccc56)c5ccc6oc7c8ccccc8c(-c8ccccc8)cc7c6c5)ccc4oc3c3ccccc23)cc1 ; c1ccc(-c2cc3c4cc(N(c5ccc(-c6ccc(-c7ccccn7)cc6)cc5)c5ccc6oc7c8ccccc8c(-c8ccccc8)cc7c6c5)ccc4oc3c3ccccc23)cc1]

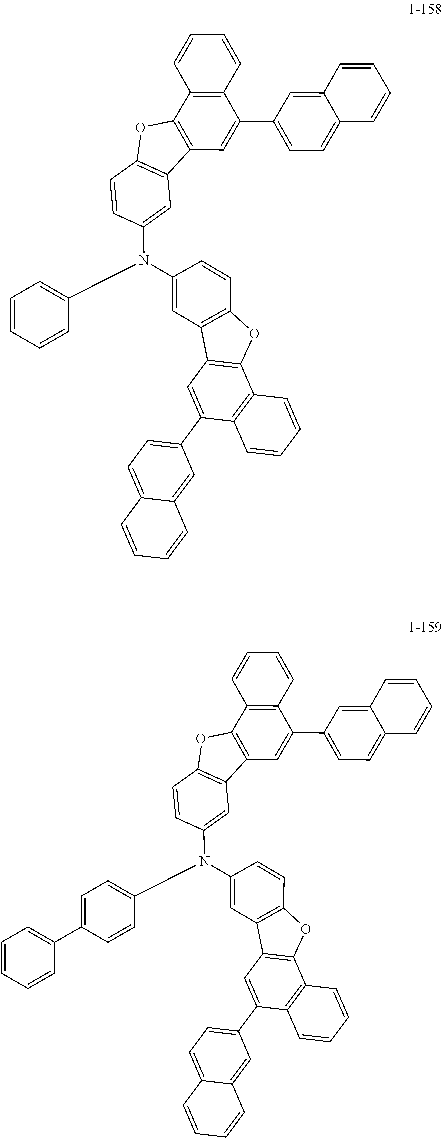 [SMILES: c1ccc(N(c2ccc3oc4c5ccccc5c(-c5ccc6ccccc6c5)cc4c3c2)c2ccc3oc4c5ccccc5c(-c5ccc6ccccc6c5)cc4c3c2)cc1 ; c1ccc(-c2ccc(N(c3ccc4oc5c6ccccc6c(-c6ccc7ccccc7c6)cc5c4c3)c3ccc4oc5c6ccccc6c(-c6ccc7ccccc7c6)cc5c4c3)cc2)cc1]

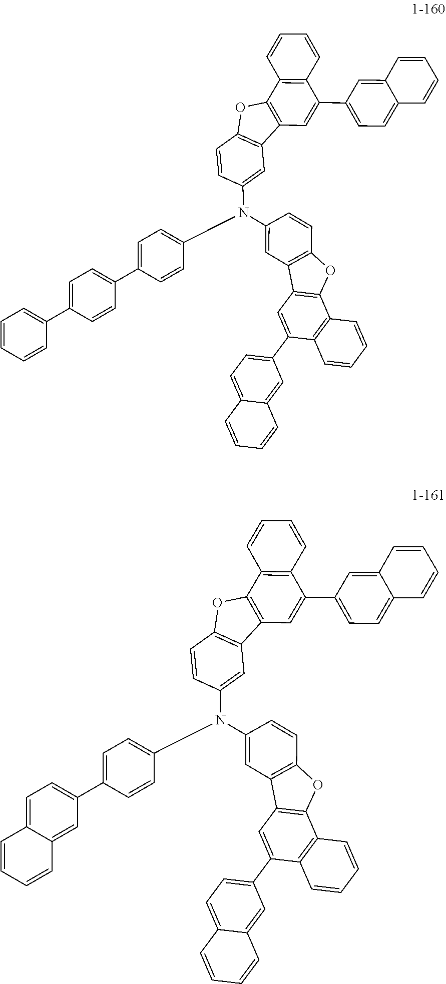 [SMILES: c1ccc(-c2ccc(-c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc3)cc2)cc1 ; c1ccc2cc(-c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc3)ccc2c1]

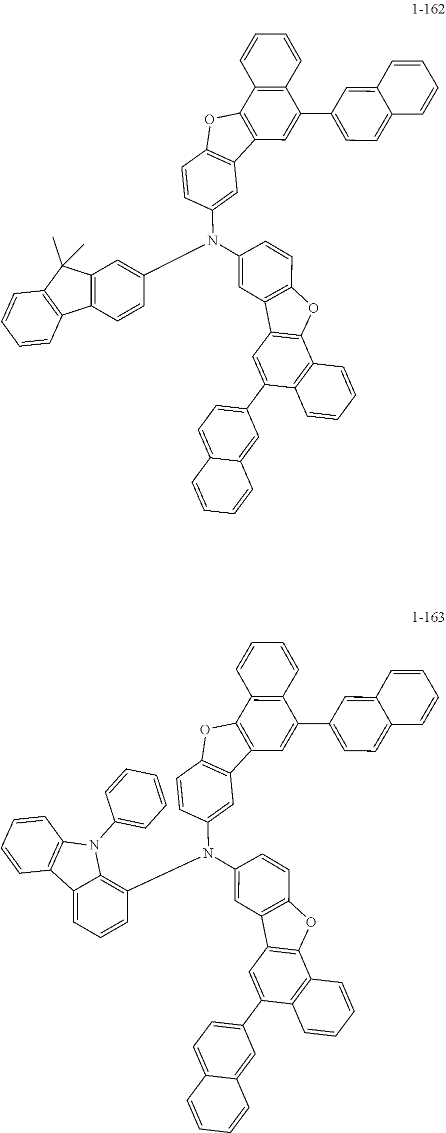 [SMILES: CC1(C)c2ccccc2-c2ccc(N(c3ccc4oc5c6ccccc6c(-c6ccc7ccccc7c6)cc5c4c3)c3ccc4oc5c6ccccc6c(-c6ccc7ccccc7c6)cc5c4c3)cc21 ; c1ccc(-n2c3ccccc3c3cccc(N(c4ccc5oc6c7ccccc7c(-c7ccc8ccccc8c7)cc6c5c4)c4ccc5oc6c7ccccc7c(-c7ccc8ccccc8c7)cc6c5c4)c32)cc1]

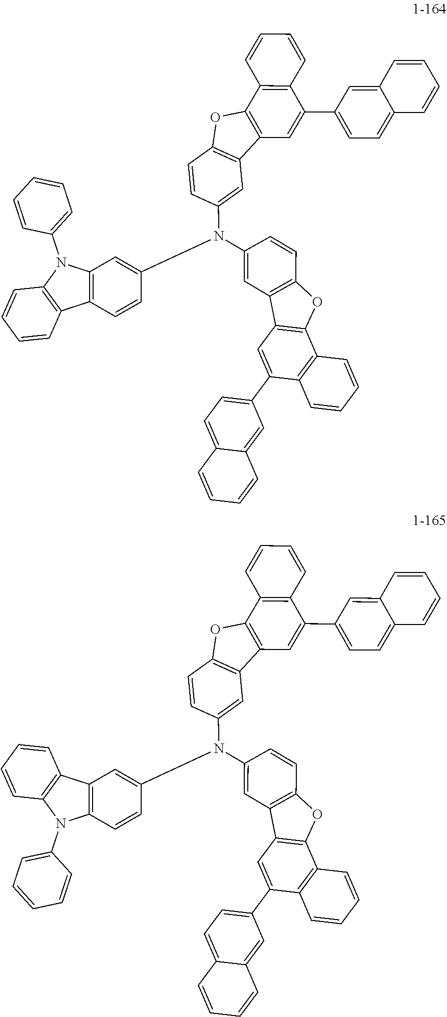 [SMILES: c1ccc(-n2c3ccccc3c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc32)cc1 ; c1ccc(-n2c3ccccc3c3cc(N(c4ccc5oc6c7ccccc7c(-c7ccc8ccccc8c7)cc6c5c4)c4ccc5oc6c7ccccc7c(-c7ccc8ccccc8c7)cc6c5c4)ccc32)cc1]

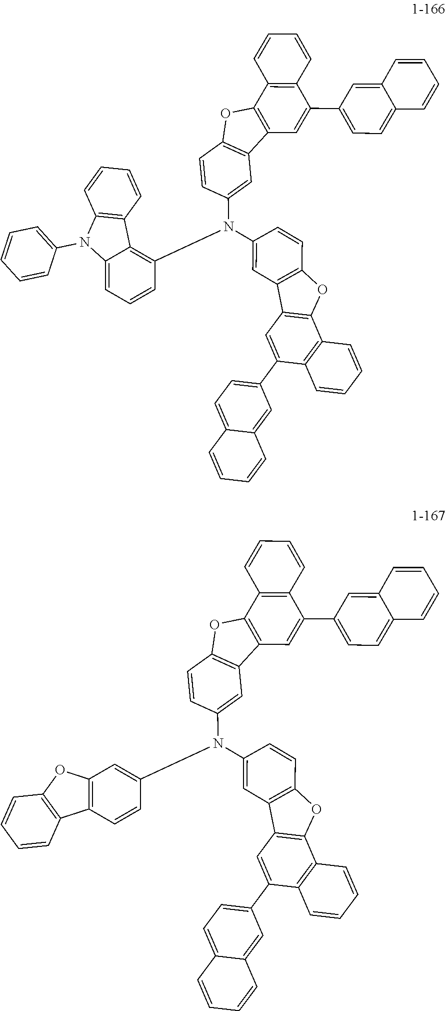 [SMILES: c1ccc(-n2c3ccccc3c3c(N(c4ccc5oc6c7ccccc7c(-c7ccc8ccccc8c7)cc6c5c4)c4ccc5oc6c7ccccc7c(-c7ccc8ccccc8c7)cc6c5c4)cccc32)cc1 ; c1ccc2cc(-c3cc4c5cc(N(c6ccc7c(c6)oc6ccccc67)c6ccc7oc8c9ccccc9c(-c9ccc%10ccccc%10c9)cc8c7c6)ccc5oc4c4ccccc34)ccc2c1]

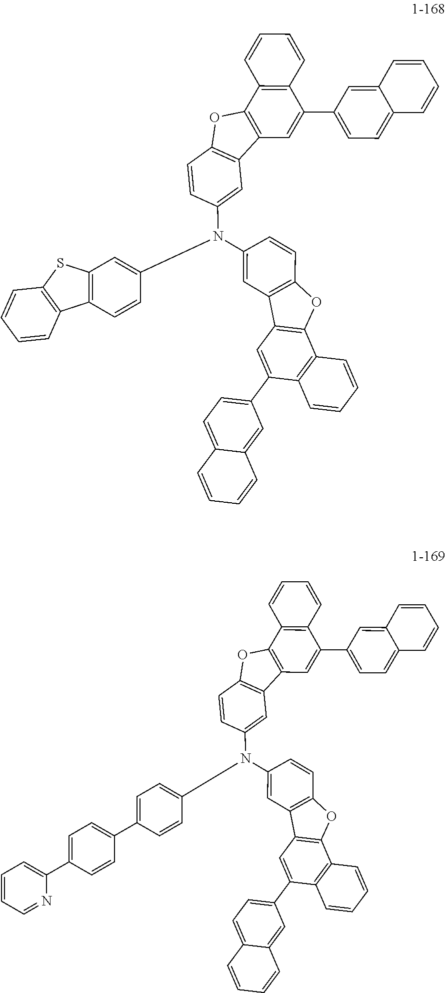 [SMILES: c1ccc2cc(-c3cc4c5cc(N(c6ccc7c(c6)sc6ccccc67)c6ccc7oc8c9ccccc9c(-c9ccc%10ccccc%10c9)cc8c7c6)ccc5oc4c4ccccc34)ccc2c1 ; c1ccc(-c2ccc(-c3ccc(N(c4ccc5oc6c7ccccc7c(-c7ccc8ccccc8c7)cc6c5c4)c4ccc5oc6c7ccccc7c(-c7ccc8ccccc8c7)cc6c5c4)cc3)cc2)nc1]

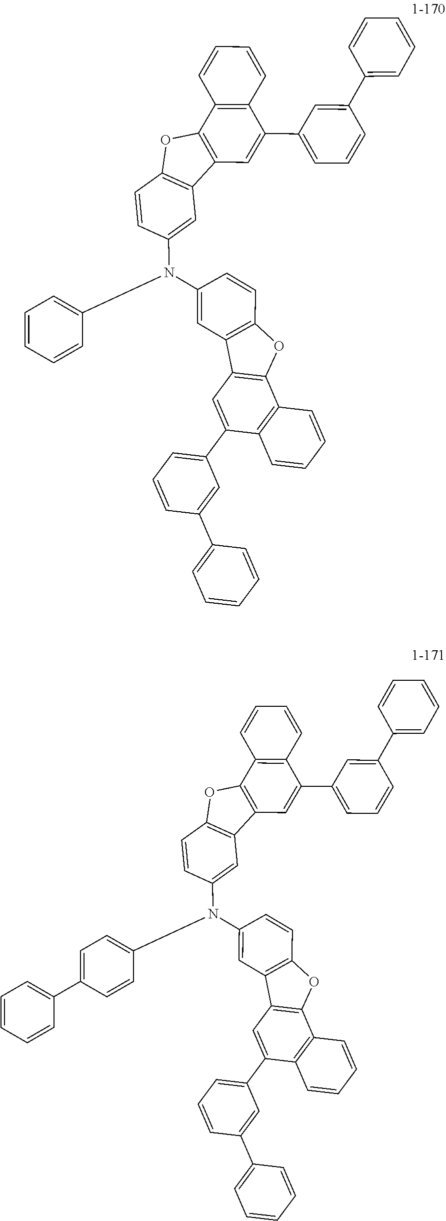 [SMILES: c1ccc(-c2cccc(-c3cc4c5cc(N(c6ccccc6)c6ccc7oc8c9ccccc9c(-c9cccc(-c%10ccccc%10)c9)cc8c7c6)ccc5oc4c4ccccc34)c2)cc1 ; c1ccc(-c2ccc(N(c3ccc4oc5c6ccccc6c(-c6cccc(-c7ccccc7)c6)cc5c4c3)c3ccc4oc5c6ccccc6c(-c6cccc(-c7ccccc7)c6)cc5c4c3)cc2)cc1]

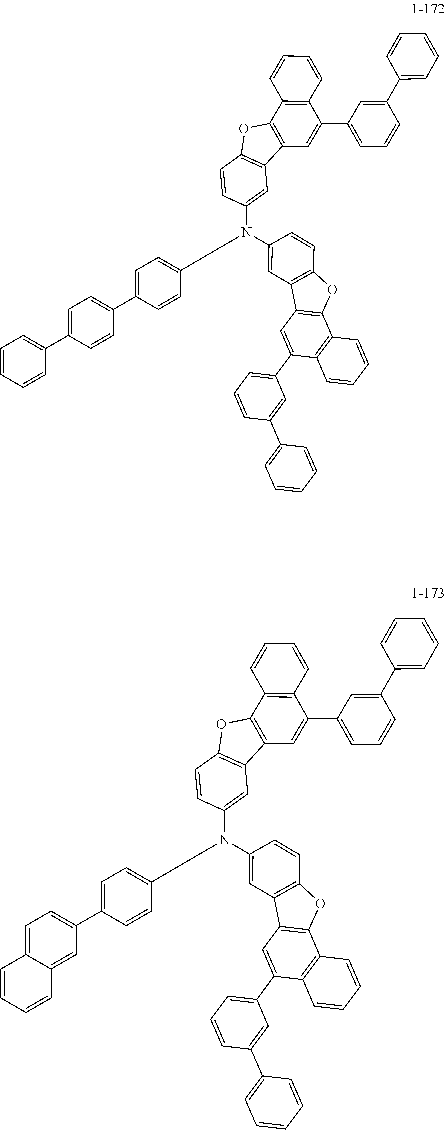 [SMILES: c1ccc(-c2ccc(-c3ccc(N(c4ccc5oc6c7ccccc7c(-c7cccc(-c8ccccc8)c7)cc6c5c4)c4ccc5oc6c7ccccc7c(-c7cccc(-c8ccccc8)c7)cc6c5c4)cc3)cc2)cc1 ; c1ccc(-c2cccc(-c3cc4c5cc(N(c6ccc(-c7ccc8ccccc8c7)cc6)c6ccc7oc8c9ccccc9c(-c9cccc(-c%10ccccc%10)c9)cc8c7c6)ccc5oc4c4ccccc34)c2)cc1]

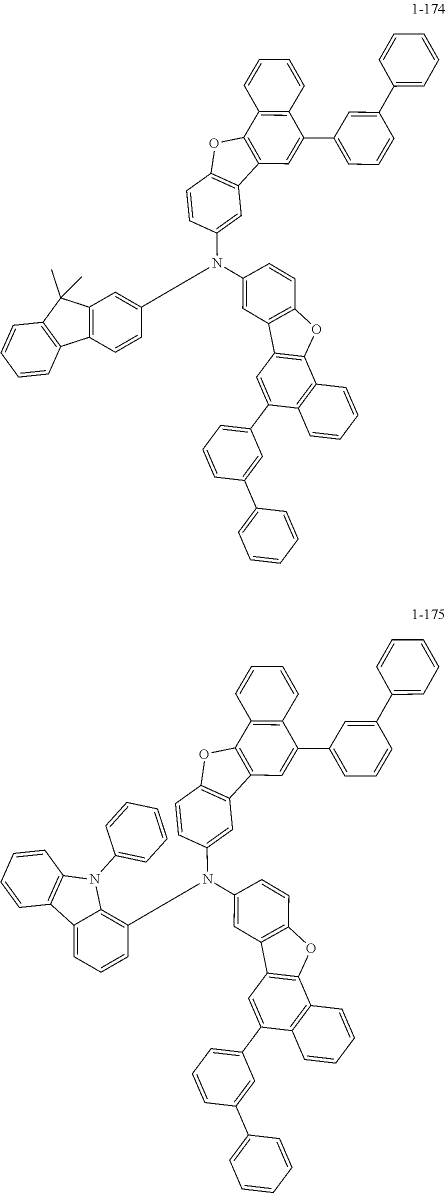 [SMILES: CC1(C)c2ccccc2-c2ccc(N(c3ccc4oc5c6ccccc6c(-c6cccc(-c7ccccc7)c6)cc5c4c3)c3ccc4oc5c6ccccc6c(-c6cccc(-c7ccccc7)c6)cc5c4c3)cc21 ; c1ccc(-c2cccc(-c3cc4c5cc(N(c6ccc7oc8c9ccccc9c(-c9cccc(-c%10ccccc%10)c9)cc8c7c6)c6cccc7c8ccccc8n(-c8ccccc8)c67)ccc5oc4c4ccccc34)c2)cc1]

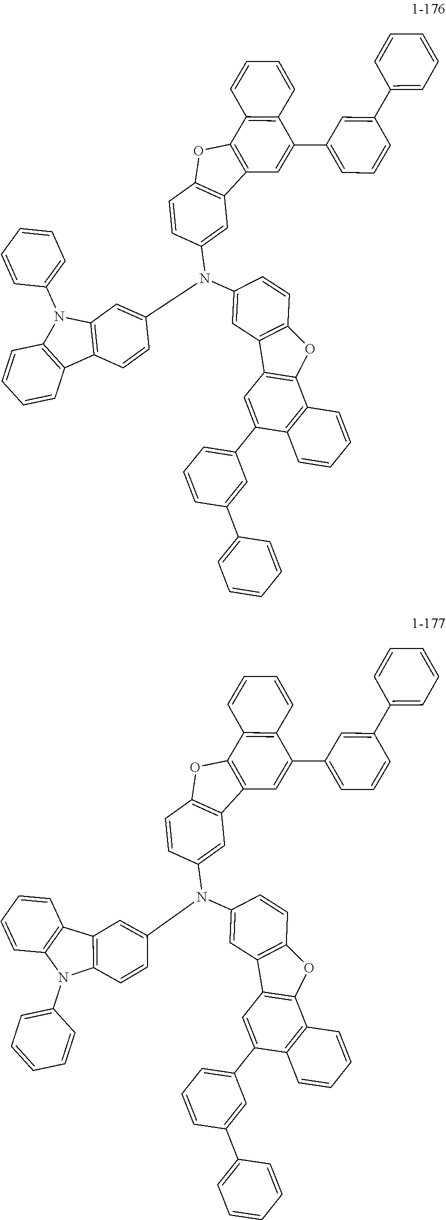 [SMILES: c1ccc(-c2cccc(-c3cc4c5cc(N(c6ccc7oc8c9ccccc9c(-c9cccc(-c%10ccccc%10)c9)cc8c7c6)c6ccc7c8ccccc8n(-c8ccccc8)c7c6)ccc5oc4c4ccccc34)c2)cc1 ; c1ccc(-c2cccc(-c3cc4c5cc(N(c6ccc7oc8c9ccccc9c(-c9cccc(-c%10ccccc%10)c9)cc8c7c6)c6ccc7c(c6)c6ccccc6n7-c6ccccc6)ccc5oc4c4ccccc34)c2)cc1]

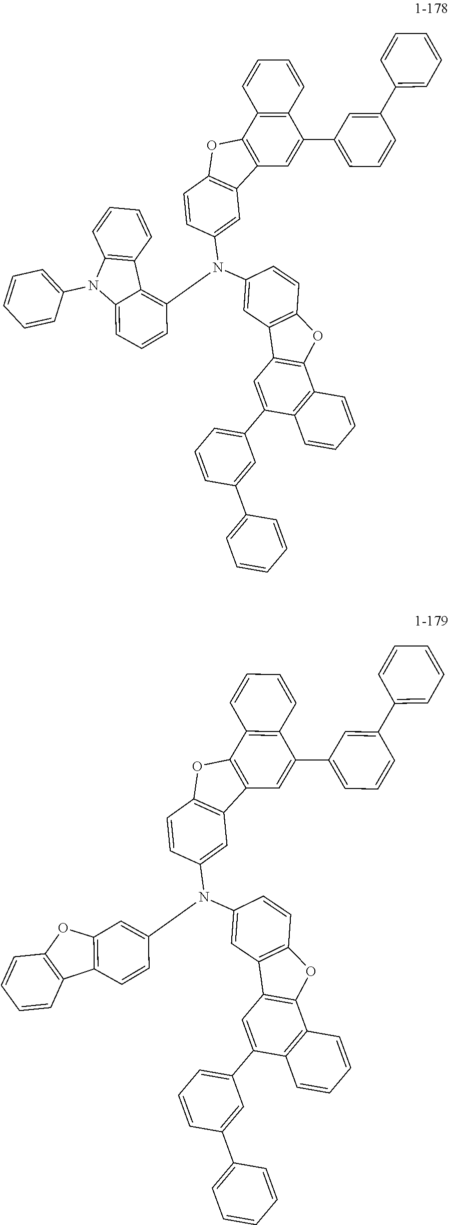 [SMILES: c1ccc(-c2cccc(-c3cc4c5cc(N(c6ccc7oc8c9ccccc9c(-c9cccc(-c%10ccccc%10)c9)cc8c7c6)c6cccc7c6c6ccccc6n7-c6ccccc6)ccc5oc4c4ccccc34)c2)cc1 ; c1ccc(-c2cccc(-c3cc4c5cc(N(c6ccc7c(c6)oc6ccccc67)c6ccc7oc8c9ccccc9c(-c9cccc(-c%10ccccc%10)c9)cc8c7c6)ccc5oc4c4ccccc34)c2)cc1]

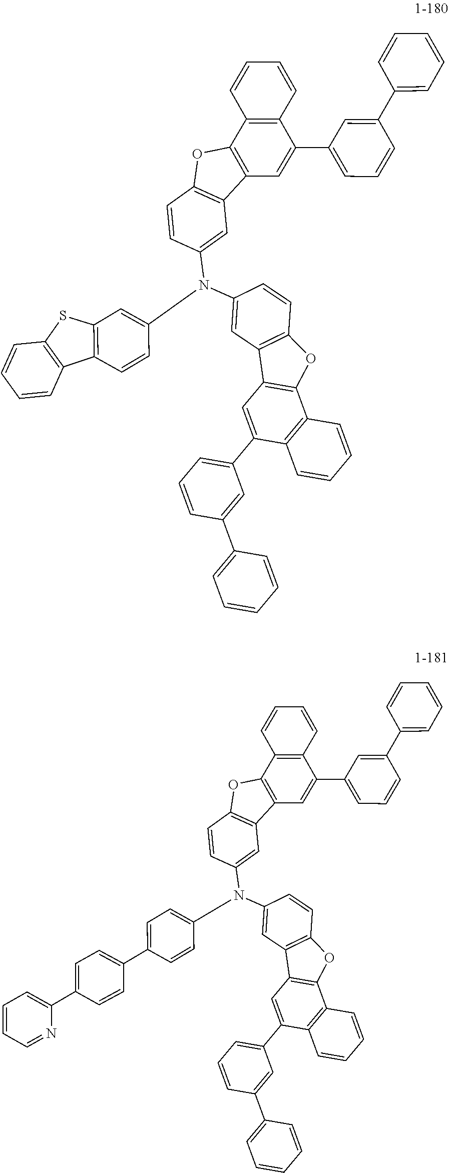 [SMILES: c1ccc(-c2cccc(-c3cc4c5cc(N(c6ccc7c(c6)sc6ccccc67)c6ccc7oc8c9ccccc9c(-c9cccc(-c%10ccccc%10)c9)cc8c7c6)ccc5oc4c4ccccc34)c2)cc1 ; c1ccc(-c2cccc(-c3cc4c5cc(N(c6ccc(-c7ccc(-c8ccccn8)cc7)cc6)c6ccc7oc8c9ccccc9c(-c9cccc(-c%10ccccc%10)c9)cc8c7c6)ccc5oc4c4ccccc34)c2)cc1]

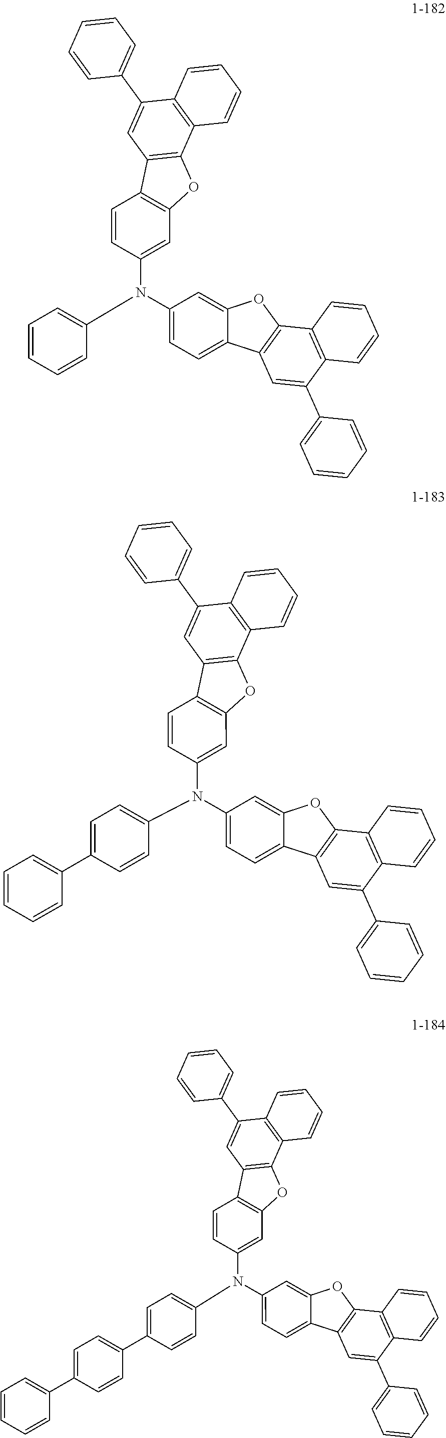 [SMILES: c1ccc(-c2cc3c4ccc(N(c5ccccc5)c5ccc6c(c5)oc5c7ccccc7c(-c7ccccc7)cc65)cc4oc3c3ccccc23)cc1 ; c1ccc(-c2ccc(N(c3ccc4c(c3)oc3c5ccccc5c(-c5ccccc5)cc43)c3ccc4c(c3)oc3c5ccccc5c(-c5ccccc5)cc43)cc2)cc1 ; c1ccc(-c2ccc(-c3ccc(N(c4ccc5c(c4)oc4c6ccccc6c(-c6ccccc6)cc54)c4ccc5c(c4)oc4c6ccccc6c(-c6ccccc6)cc54)cc3)cc2)cc1]

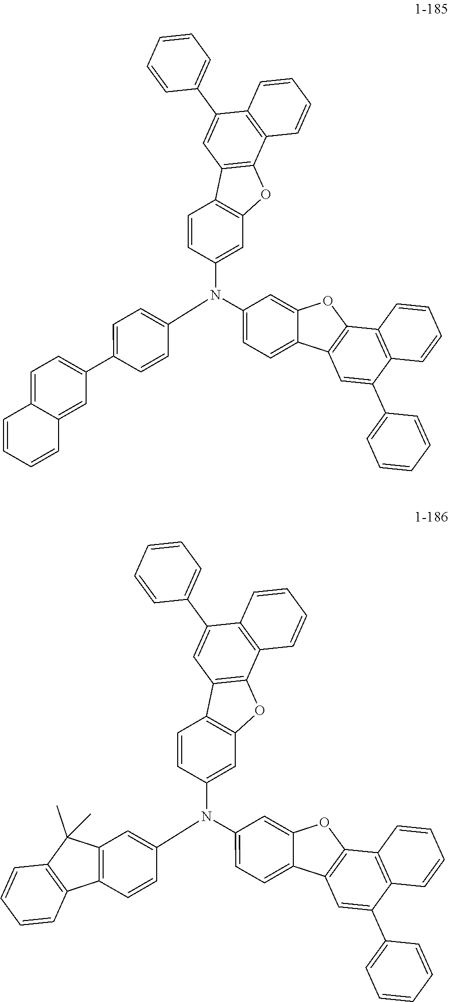 [SMILES: c1ccc(-c2cc3c4ccc(N(c5ccc(-c6ccc7ccccc7c6)cc5)c5ccc6c(c5)oc5c7ccccc7c(-c7ccccc7)cc65)cc4oc3c3ccccc23)cc1 ; CC1(C)c2ccccc2-c2ccc(N(c3ccc4c(c3)oc3c5ccccc5c(-c5ccccc5)cc43)c3ccc4c(c3)oc3c5ccccc5c(-c5ccccc5)cc43)cc21]

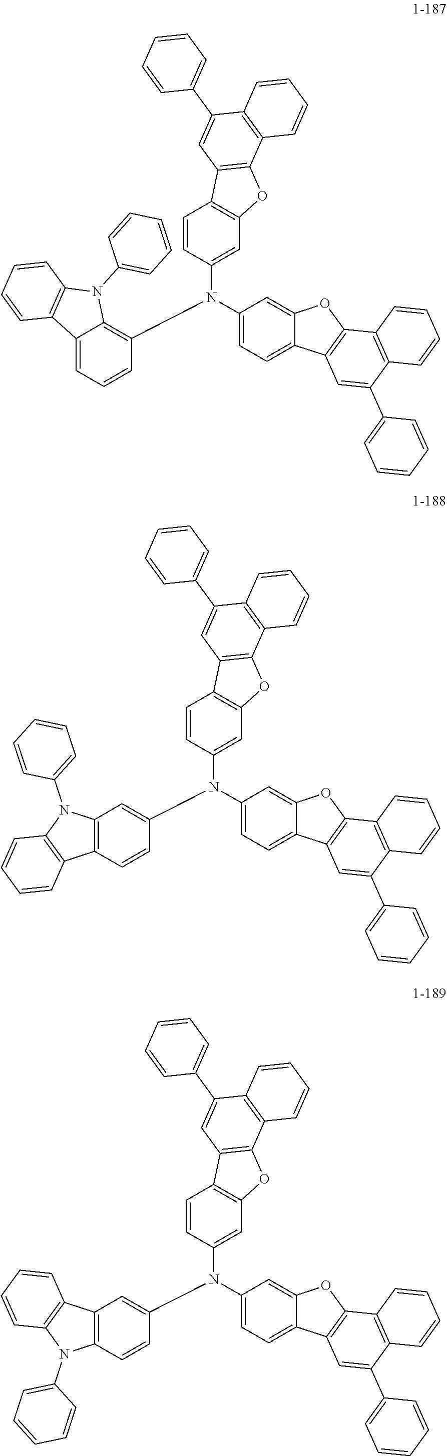 [SMILES: c1ccc(-c2cc3c4ccc(N(c5ccc6c(c5)oc5c7ccccc7c(-c7ccccc7)cc65)c5cccc6c7ccccc7n(-c7ccccc7)c56)cc4oc3c3ccccc23)cc1 ; c1ccc(-c2cc3c4ccc(N(c5ccc6c(c5)oc5c7ccccc7c(-c7ccccc7)cc65)c5ccc6c7ccccc7n(-c7ccccc7)c6c5)cc4oc3c3ccccc23)cc1 ; c1ccc(-c2cc3c4ccc(N(c5ccc6c(c5)oc5c7ccccc7c(-c7ccccc7)cc65)c5ccc6c(c5)c5ccccc5n6-c5ccccc5)cc4oc3c3ccccc23)cc1]

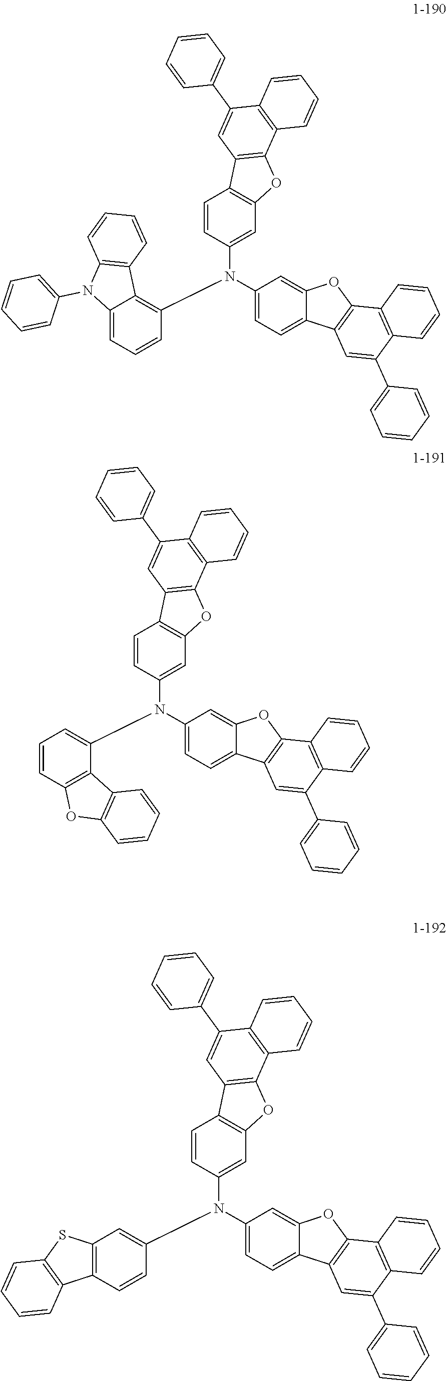 [SMILES: c1ccc(-c2cc3c4ccc(N(c5ccc6c(c5)oc5c7ccccc7c(-c7ccccc7)cc65)c5cccc6c5c5ccccc5n6-c5ccccc5)cc4oc3c3ccccc23)cc1 ; c1ccc(-c2cc3c4ccc(N(c5ccc6c(c5)oc5c7ccccc7c(-c7ccccc7)cc65)c5cccc6oc7ccccc7c56)cc4oc3c3ccccc23)cc1 ; c1ccc(-c2cc3c4ccc(N(c5ccc6c(c5)oc5c7ccccc7c(-c7ccccc7)cc65)c5ccc6c(c5)sc5ccccc56)cc4oc3c3ccccc23)cc1]

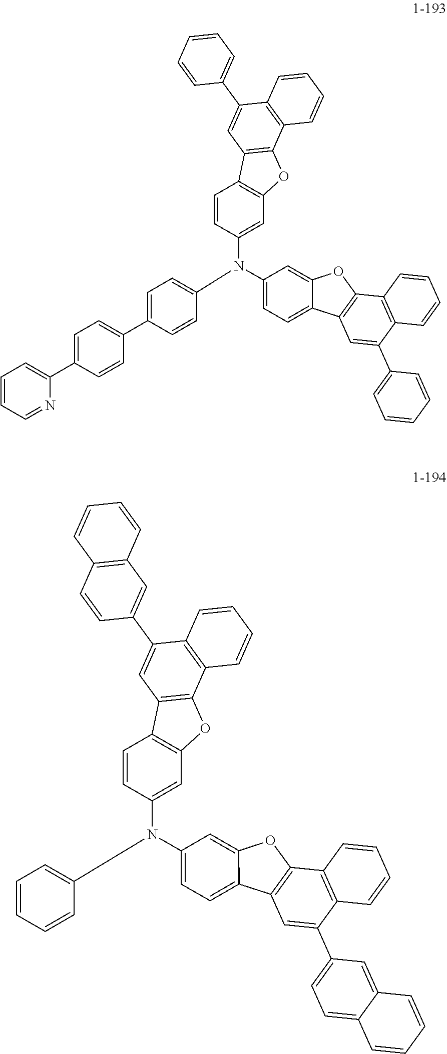 [SMILES: c1ccc(-c2cc3c4ccc(N(c5ccc(-c6ccc(-c7ccccn7)cc6)cc5)c5ccc6c(c5)oc5c7ccccc7c(-c7ccccc7)cc65)cc4oc3c3ccccc23)cc1 ; c1ccc(N(c2ccc3c(c2)oc2c4ccccc4c(-c4ccc5ccccc5c4)cc32)c2ccc3c(c2)oc2c4ccccc4c(-c4ccc5ccccc5c4)cc32)cc1]

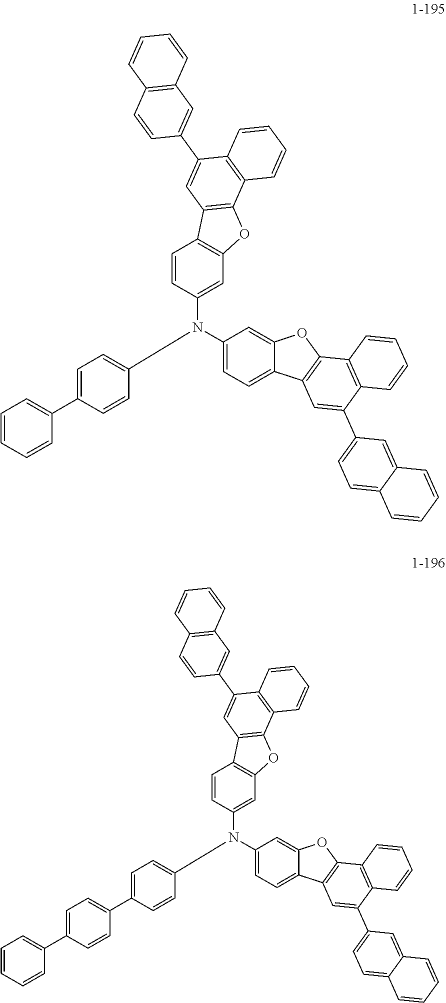 [SMILES: c1ccc(-c2ccc(N(c3ccc4c(c3)oc3c5ccccc5c(-c5ccc6ccccc6c5)cc43)c3ccc4c(c3)oc3c5ccccc5c(-c5ccc6ccccc6c5)cc43)cc2)cc1 ; c1ccc(-c2ccc(-c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc3)cc2)cc1]

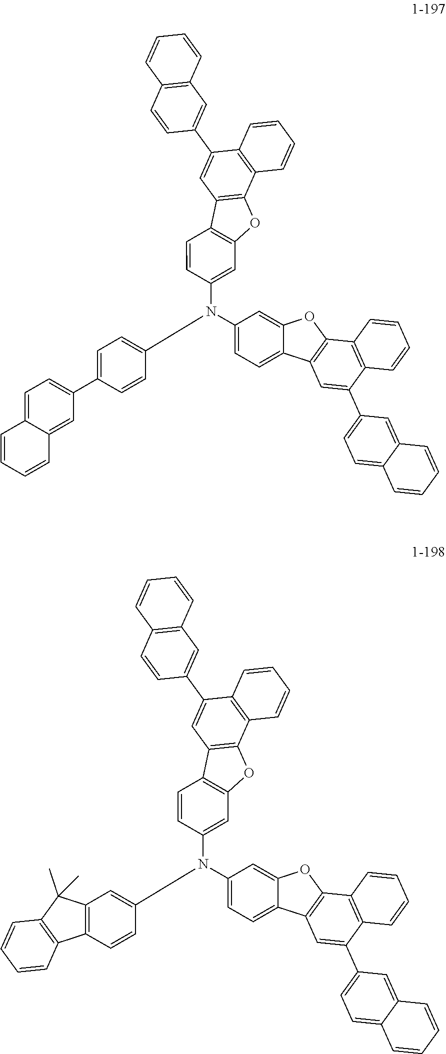 [SMILES: c1ccc2cc(-c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc3)ccc2c1 ; CC1(C)c2ccccc2-c2ccc(N(c3ccc4c(c3)oc3c5ccccc5c(-c5ccc6ccccc6c5)cc43)c3ccc4c(c3)oc3c5ccccc5c(-c5ccc6ccccc6c5)cc43)cc21]

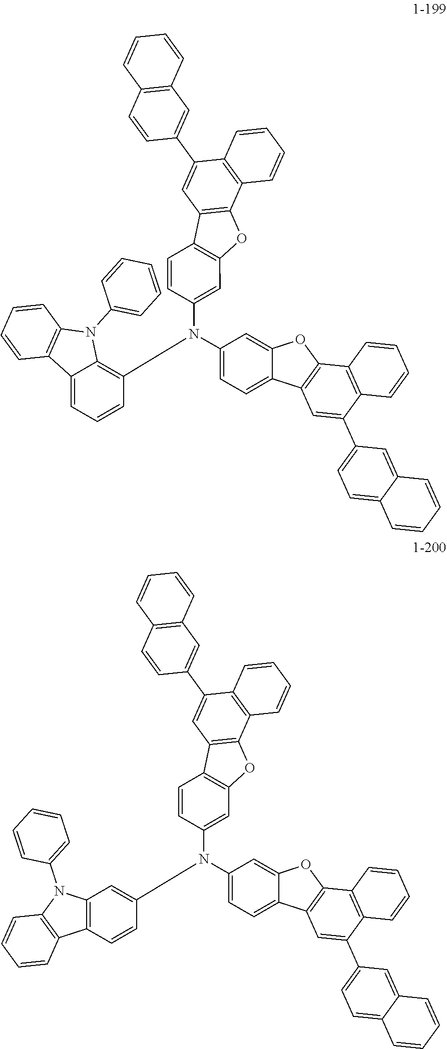 [SMILES: c1ccc(-n2c3ccccc3c3cccc(N(c4ccc5c(c4)oc4c6ccccc6c(-c6ccc7ccccc7c6)cc54)c4ccc5c(c4)oc4c6ccccc6c(-c6ccc7ccccc7c6)cc54)c32)cc1 ; c1ccc(-n2c3ccccc3c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc32)cc1]

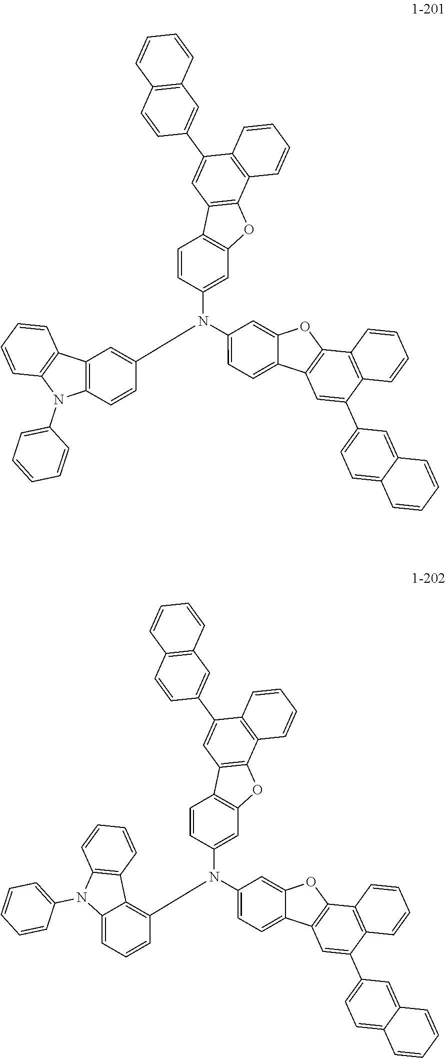 [SMILES: c1ccc(-n2c3ccccc3c3cc(N(c4ccc5c(c4)oc4c6ccccc6c(-c6ccc7ccccc7c6)cc54)c4ccc5c(c4)oc4c6ccccc6c(-c6ccc7ccccc7c6)cc54)ccc32)cc1 ; c1ccc(-n2c3ccccc3c3c(N(c4ccc5c(c4)oc4c6ccccc6c(-c6ccc7ccccc7c6)cc54)c4ccc5c(c4)oc4c6ccccc6c(-c6ccc7ccccc7c6)cc54)cccc32)cc1]

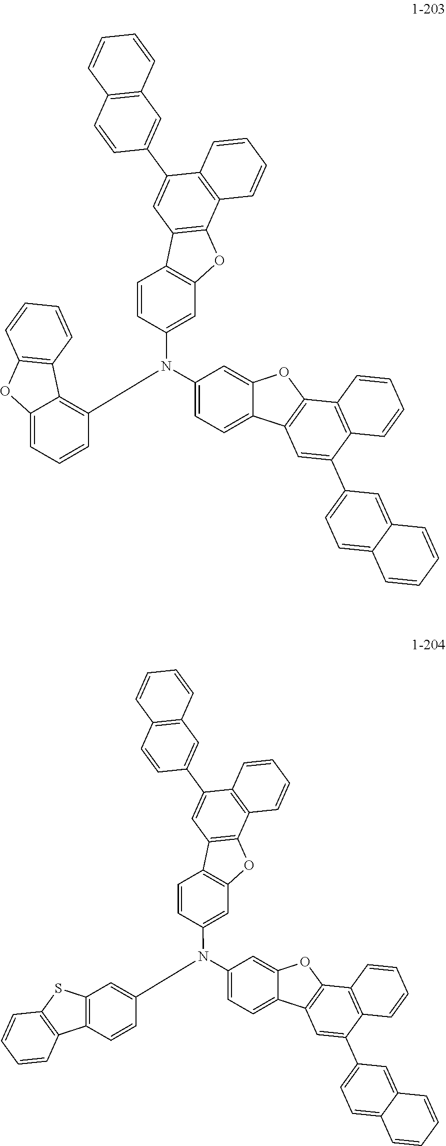 [SMILES: c1ccc2cc(-c3cc4c5ccc(N(c6ccc7c(c6)oc6c8ccccc8c(-c8ccc9ccccc9c8)cc76)c6cccc7oc8ccccc8c67)cc5oc4c4ccccc34)ccc2c1 ; c1ccc2cc(-c3cc4c5ccc(N(c6ccc7c(c6)oc6c8ccccc8c(-c8ccc9ccccc9c8)cc76)c6ccc7c(c6)sc6ccccc67)cc5oc4c4ccccc34)ccc2c1]

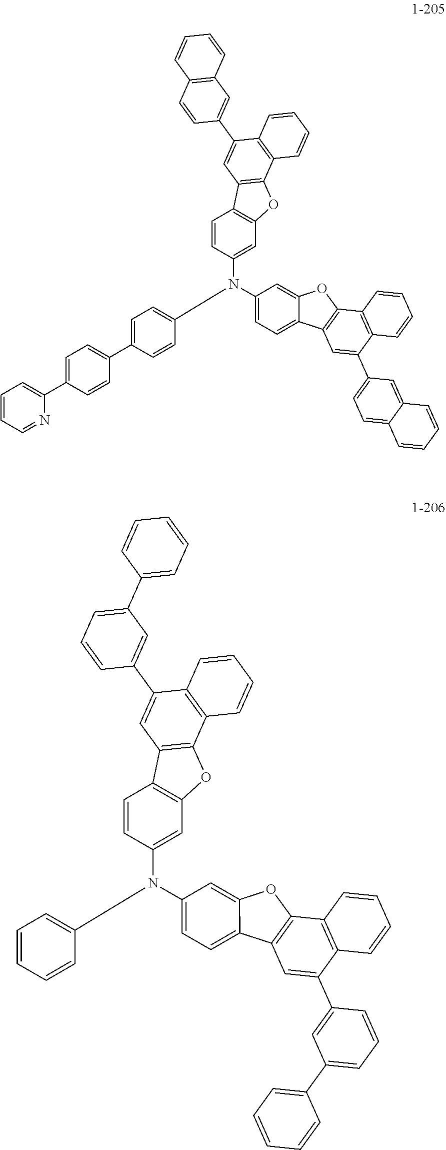 [SMILES: c1ccc(-c2ccc(-c3ccc(N(c4ccc5c(c4)oc4c6ccccc6c(-c6ccc7ccccc7c6)cc54)c4ccc5c(c4)oc4c6ccccc6c(-c6ccc7ccccc7c6)cc54)cc3)cc2)nc1 ; c1ccc(-c2cccc(-c3cc4c5ccc(N(c6ccccc6)c6ccc7c(c6)oc6c8ccccc8c(-c8cccc(-c9ccccc9)c8)cc76)cc5oc4c4ccccc34)c2)cc1]

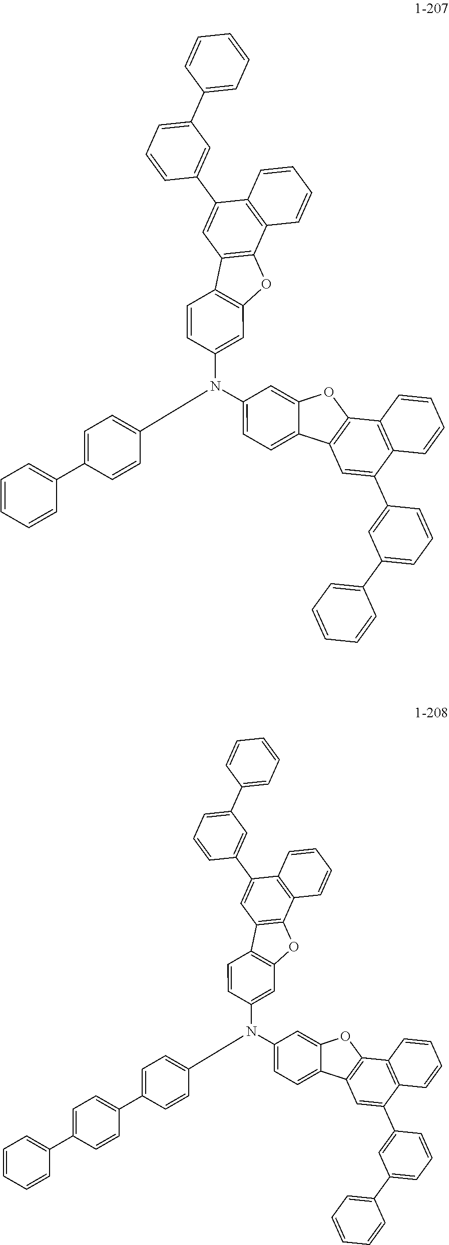 [SMILES: c1ccc(-c2ccc(N(c3ccc4c(c3)oc3c5ccccc5c(-c5cccc(-c6ccccc6)c5)cc43)c3ccc4c(c3)oc3c5ccccc5c(-c5cccc(-c6ccccc6)c5)cc43)cc2)cc1 ; c1ccc(-c2ccc(-c3ccc(N(c4ccc5c(c4)oc4c6ccccc6c(-c6cccc(-c7ccccc7)c6)cc54)c4ccc5c(c4)oc4c6ccccc6c(-c6cccc(-c7ccccc7)c6)cc54)cc3)cc2)cc1]

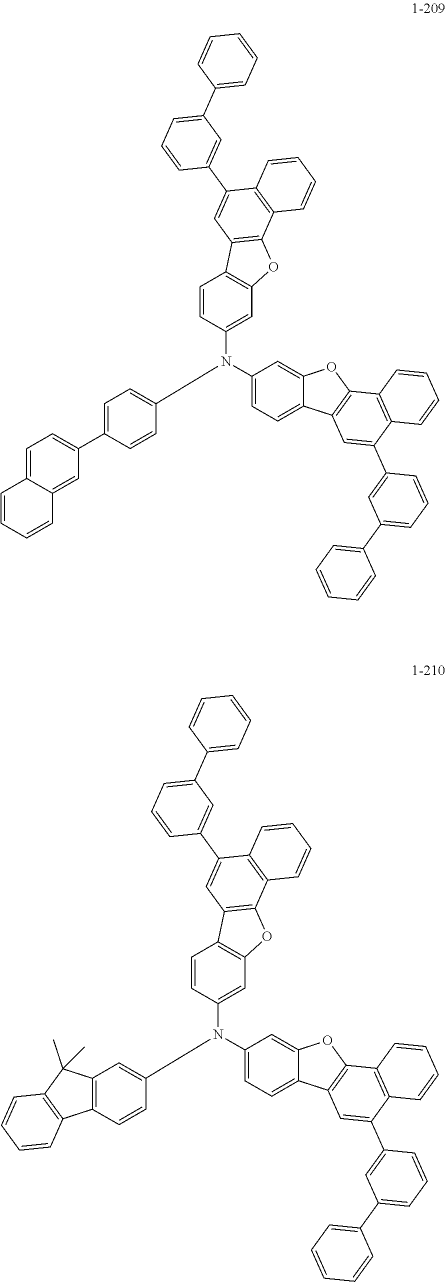 [SMILES: c1ccc(-c2cccc(-c3cc4c5ccc(N(c6ccc(-c7ccc8ccccc8c7)cc6)c6ccc7c(c6)oc6c8ccccc8c(-c8cccc(-c9ccccc9)c8)cc76)cc5oc4c4ccccc34)c2)cc1 ; CC1(C)c2ccccc2-c2ccc(N(c3ccc4c(c3)oc3c5ccccc5c(-c5cccc(-c6ccccc6)c5)cc43)c3ccc4c(c3)oc3c5ccccc5c(-c5cccc(-c6ccccc6)c5)cc43)cc21]

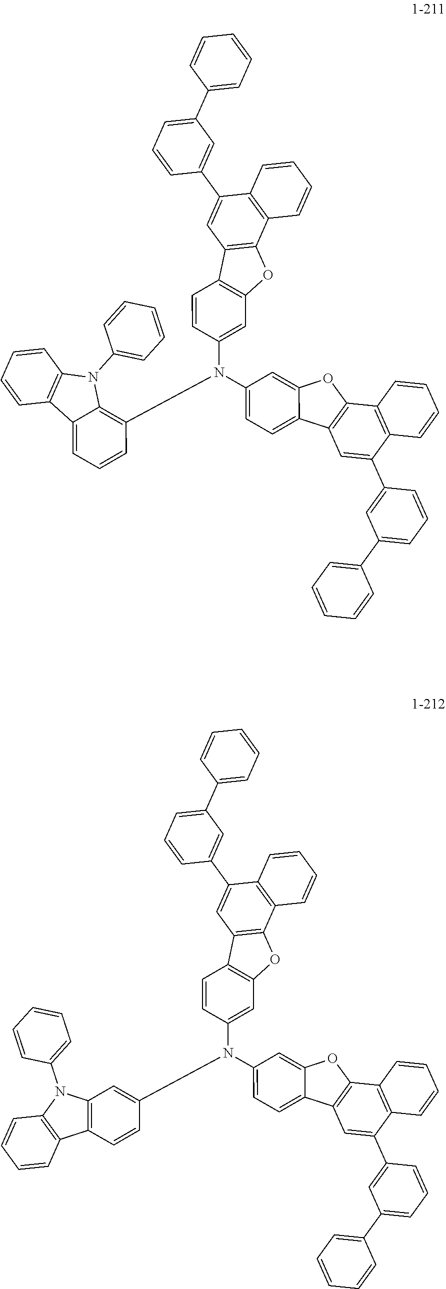 [SMILES: c1ccc(-c2cccc(-c3cc4c5ccc(N(c6ccc7c(c6)oc6c8ccccc8c(-c8cccc(-c9ccccc9)c8)cc76)c6cccc7c8ccccc8n(-c8ccccc8)c67)cc5oc4c4ccccc34)c2)cc1 ; c1ccc(-c2cccc(-c3cc4c5ccc(N(c6ccc7c(c6)oc6c8ccccc8c(-c8cccc(-c9ccccc9)c8)cc76)c6ccc7c8ccccc8n(-c8ccccc8)c7c6)cc5oc4c4ccccc34)c2)cc1]

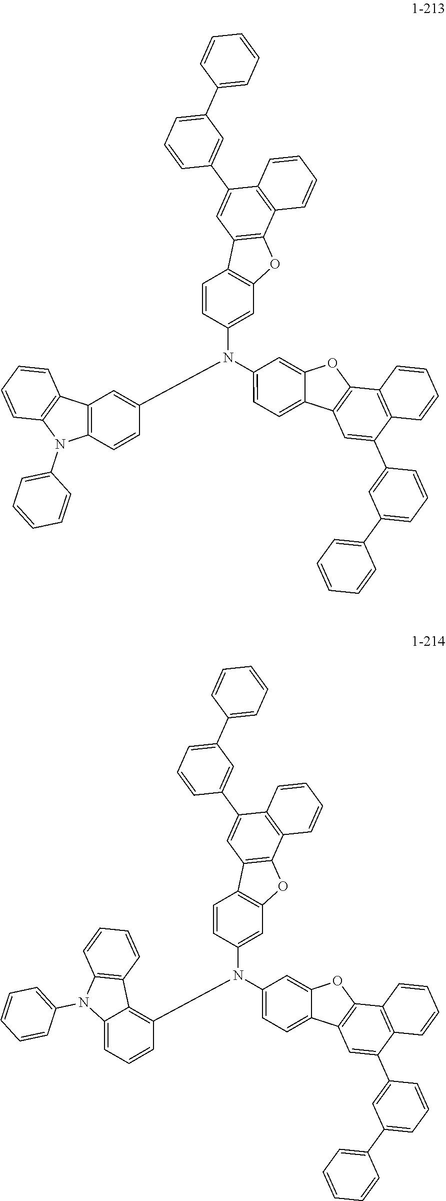 [SMILES: c1ccc(-c2cccc(-c3cc4c5ccc(N(c6ccc7c(c6)oc6c8ccccc8c(-c8cccc(-c9ccccc9)c8)cc76)c6ccc7c(c6)c6ccccc6n7-c6ccccc6)cc5oc4c4ccccc34)c2)cc1 ; c1ccc(-c2cccc(-c3cc4c5ccc(N(c6ccc7c(c6)oc6c8ccccc8c(-c8cccc(-c9ccccc9)c8)cc76)c6cccc7c6c6ccccc6n7-c6ccccc6)cc5oc4c4ccccc34)c2)cc1]

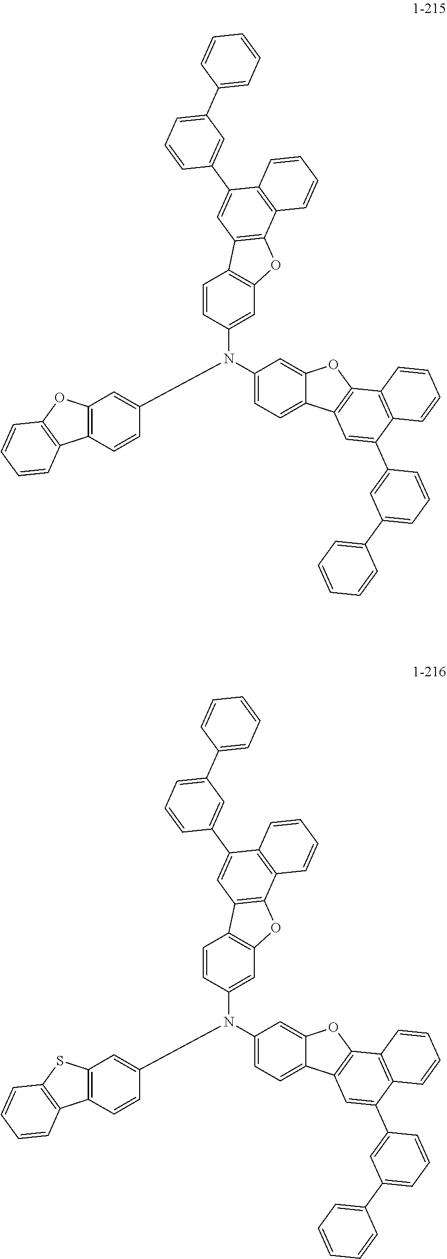 [SMILES: c1ccc(-c2cccc(-c3cc4c5ccc(N(c6ccc7c(c6)oc6ccccc67)c6ccc7c(c6)oc6c8ccccc8c(-c8cccc(-c9ccccc9)c8)cc76)cc5oc4c4ccccc34)c2)cc1 ; c1ccc(-c2cccc(-c3cc4c5ccc(N(c6ccc7c(c6)oc6c8ccccc8c(-c8cccc(-c9ccccc9)c8)cc76)c6ccc7c(c6)sc6ccccc67)cc5oc4c4ccccc34)c2)cc1]

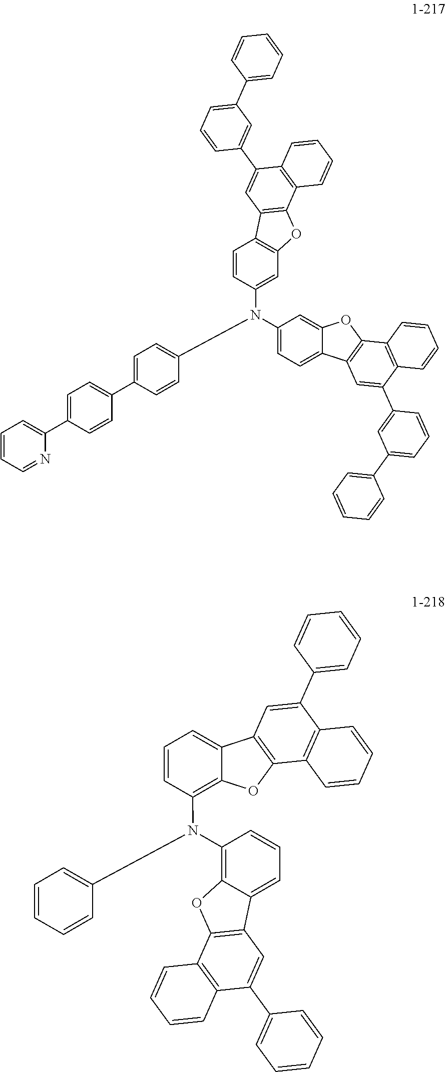 [SMILES: c1ccc(-c2cccc(-c3cc4c5ccc(N(c6ccc(-c7ccc(-c8ccccn8)cc7)cc6)c6ccc7c(c6)oc6c8ccccc8c(-c8cccc(-c9ccccc9)c8)cc76)cc5oc4c4ccccc34)c2)cc1 ; c1ccc(-c2cc3c4cccc(N(c5ccccc5)c5cccc6c5oc5c7ccccc7c(-c7ccccc7)cc65)c4oc3c3ccccc23)cc1]

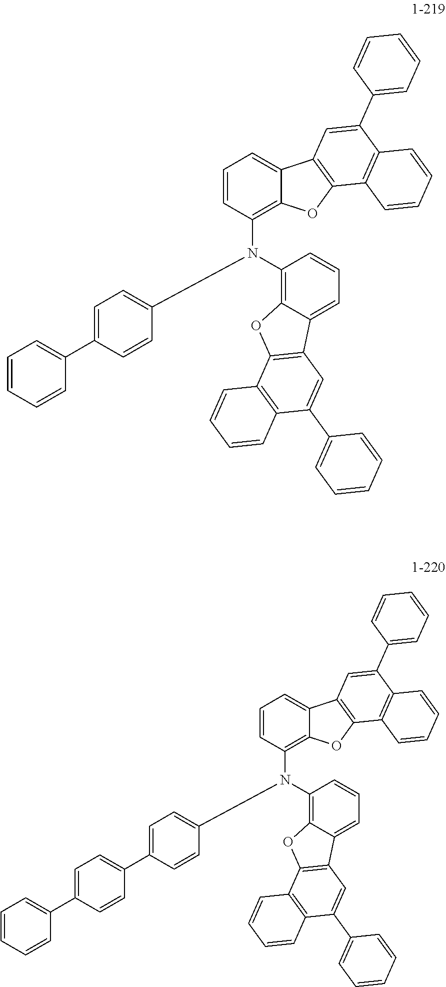 [SMILES: c1ccc(-c2ccc(N(c3cccc4c3oc3c5ccccc5c(-c5ccccc5)cc43)c3cccc4c3oc3c5ccccc5c(-c5ccccc5)cc43)cc2)cc1 ; c1ccc(-c2ccc(-c3ccc(N(c4cccc5c4oc4c6ccccc6c(-c6ccccc6)cc54)c4cccc5c4oc4c6ccccc6c(-c6ccccc6)cc54)cc3)cc2)cc1]

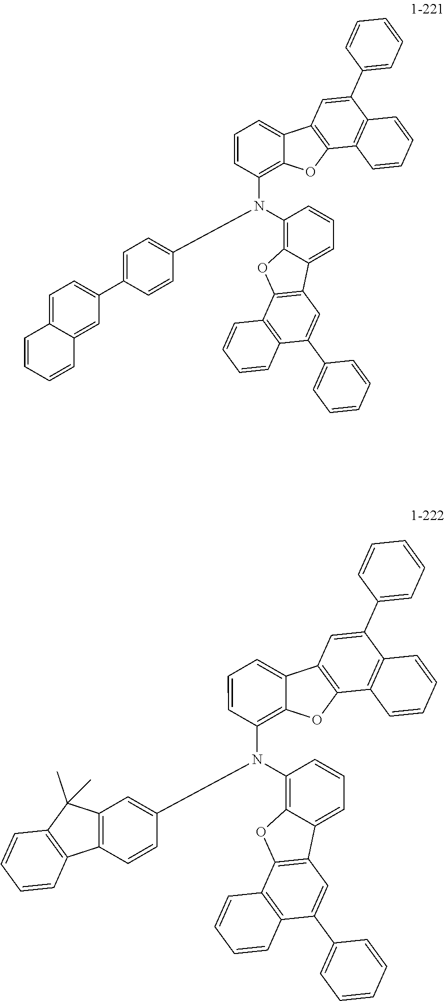 [SMILES: c1ccc(-c2cc3c4cccc(N(c5ccc(-c6ccc7ccccc7c6)cc5)c5cccc6c5oc5c7ccccc7c(-c7ccccc7)cc65)c4oc3c3ccccc23)cc1 ; CC1(C)c2ccccc2-c2ccc(N(c3cccc4c3oc3c5ccccc5c(-c5ccccc5)cc43)c3cccc4c3oc3c5ccccc5c(-c5ccccc5)cc43)cc21]

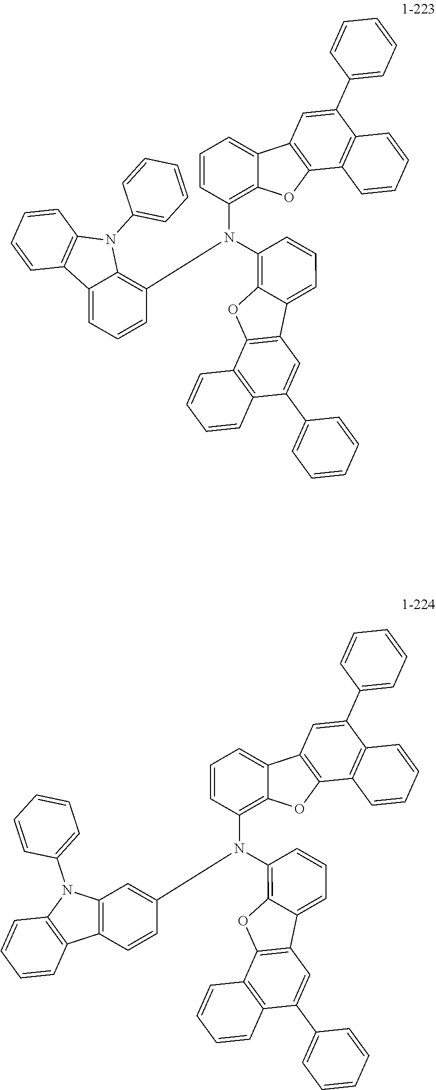 [SMILES: c1ccc(-c2cc3c4cccc(N(c5cccc6c5oc5c7ccccc7c(-c7ccccc7)cc65)c5cccc6c7ccccc7n(-c7ccccc7)c56)c4oc3c3ccccc23)cc1 ; c1ccc(-c2cc3c4cccc(N(c5ccc6c7ccccc7n(-c7ccccc7)c6c5)c5cccc6c5oc5c7ccccc7c(-c7ccccc7)cc65)c4oc3c3ccccc23)cc1]

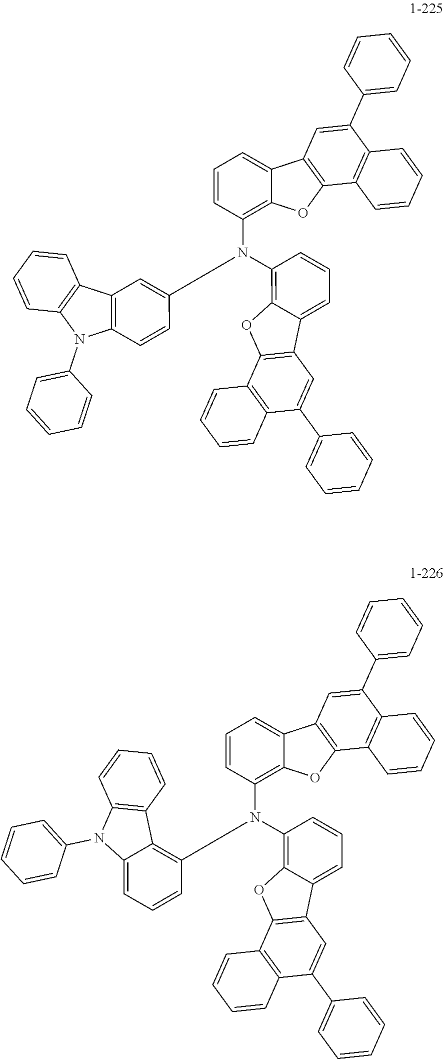 [SMILES: c1ccc(-c2cc3c4cccc(N(c5ccc6c(c5)c5ccccc5n6-c5ccccc5)c5cccc6c5oc5c7ccccc7c(-c7ccccc7)cc65)c4oc3c3ccccc23)cc1 ; c1ccc(-c2cc3c4cccc(N(c5cccc6c5oc5c7ccccc7c(-c7ccccc7)cc65)c5cccc6c5c5ccccc5n6-c5ccccc5)c4oc3c3ccccc23)cc1]

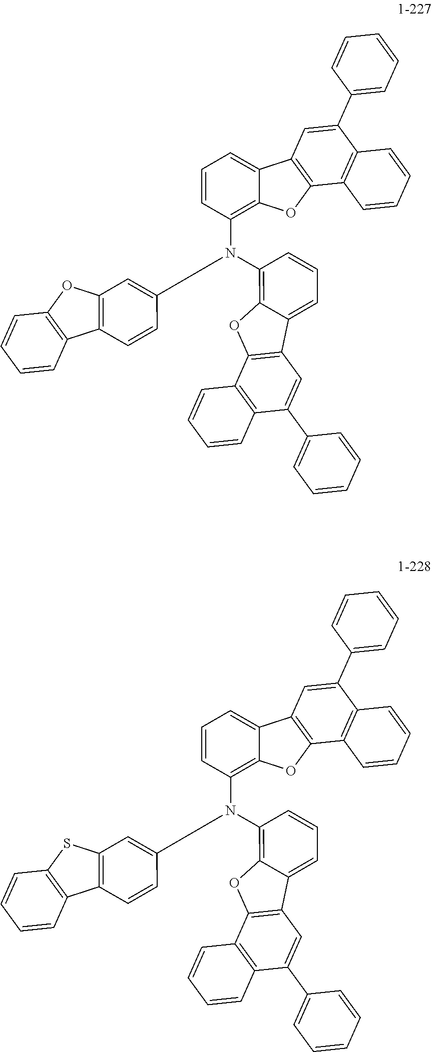 [SMILES: c1ccc(-c2cc3c4cccc(N(c5ccc6c(c5)oc5ccccc56)c5cccc6c5oc5c7ccccc7c(-c7ccccc7)cc65)c4oc3c3ccccc23)cc1 ; c1ccc(-c2cc3c4cccc(N(c5ccc6c(c5)sc5ccccc56)c5cccc6c5oc5c7ccccc7c(-c7ccccc7)cc65)c4oc3c3ccccc23)cc1]

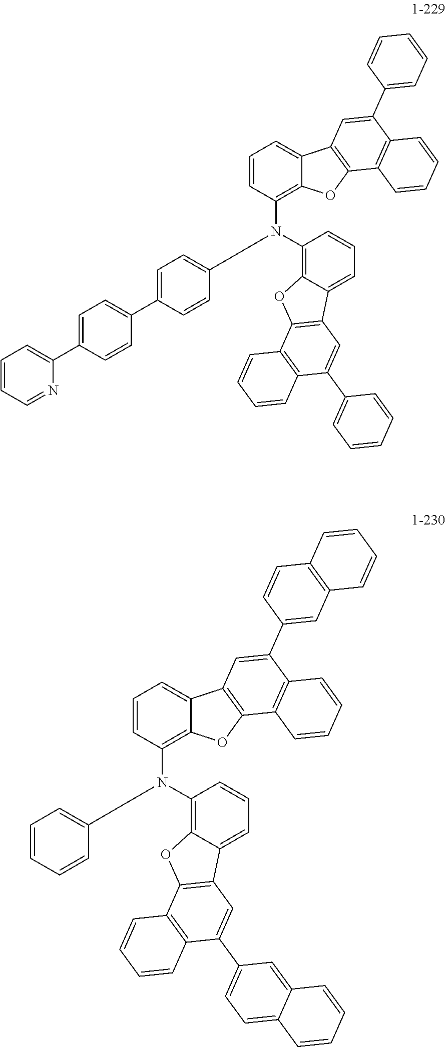 [SMILES: c1ccc(-c2cc3c4cccc(N(c5ccc(-c6ccc(-c7ccccn7)cc6)cc5)c5cccc6c5oc5c7ccccc7c(-c7ccccc7)cc65)c4oc3c3ccccc23)cc1 ; c1ccc(N(c2cccc3c2oc2c4ccccc4c(-c4ccc5ccccc5c4)cc32)c2cccc3c2oc2c4ccccc4c(-c4ccc5ccccc5c4)cc32)cc1]

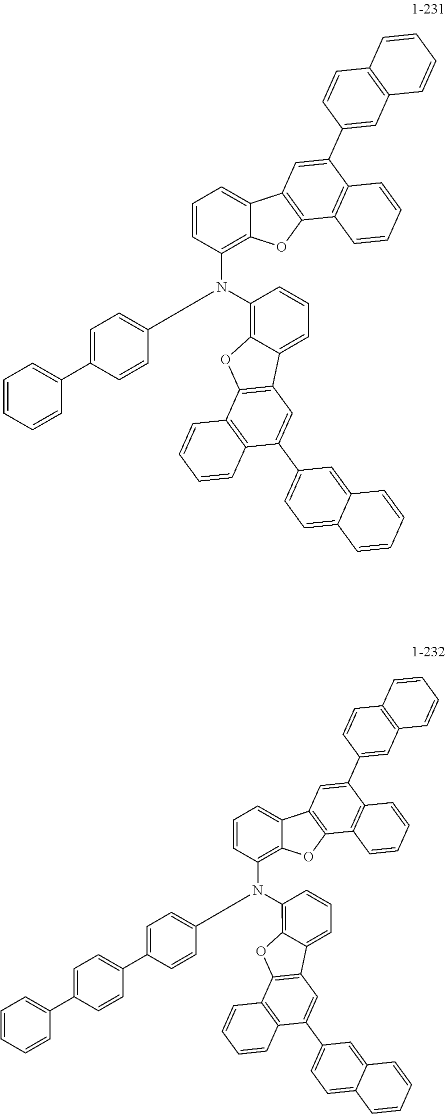 [SMILES: c1ccc(-c2ccc(N(c3cccc4c3oc3c5ccccc5c(-c5ccc6ccccc6c5)cc43)c3cccc4c3oc3c5ccccc5c(-c5ccc6ccccc6c5)cc43)cc2)cc1 ; c1ccc(-c2ccc(-c3ccc(N(c4cccc5c4oc4c6ccccc6c(-c6ccc7ccccc7c6)cc54)c4cccc5c4oc4c6ccccc6c(-c6ccc7ccccc7c6)cc54)cc3)cc2)cc1]

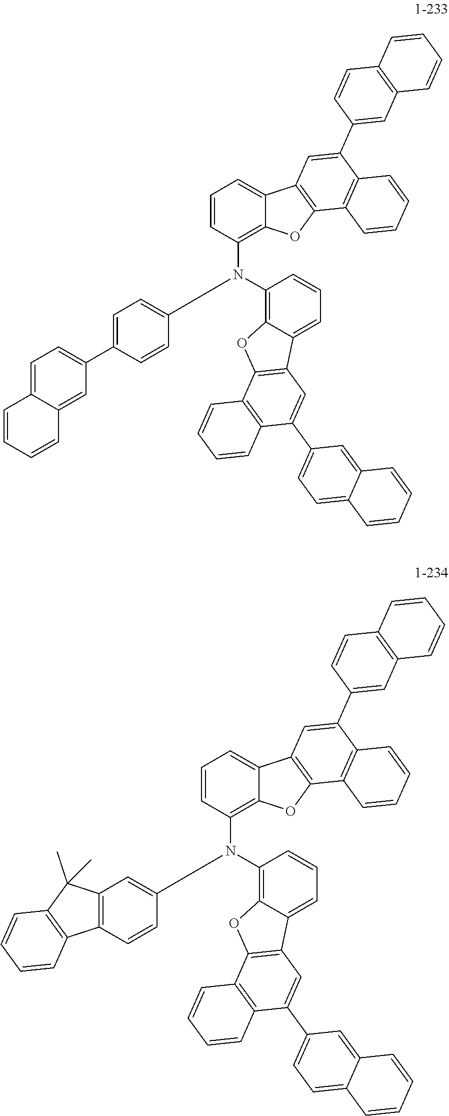 [SMILES: c1ccc2cc(-c3ccc(N(c4cccc5c4oc4c6ccccc6c(-c6ccc7ccccc7c6)cc54)c4cccc5c4oc4c6ccccc6c(-c6ccc7ccccc7c6)cc54)cc3)ccc2c1 ; CC1(C)c2ccccc2-c2ccc(N(c3cccc4c3oc3c5ccccc5c(-c5ccc6ccccc6c5)cc43)c3cccc4c3oc3c5ccccc5c(-c5ccc6ccccc6c5)cc43)cc21]

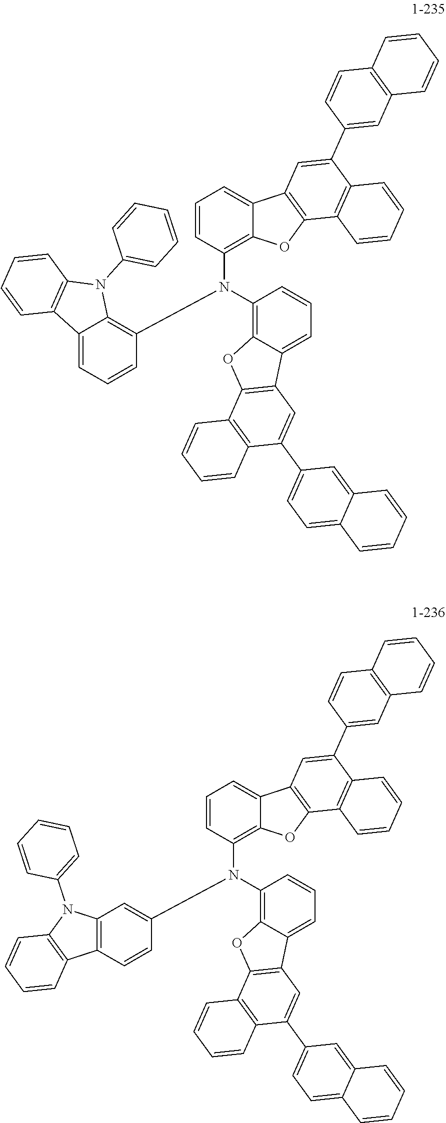 [SMILES: c1ccc(-n2c3ccccc3c3cccc(N(c4cccc5c4oc4c6ccccc6c(-c6ccc7ccccc7c6)cc54)c4cccc5c4oc4c6ccccc6c(-c6ccc7ccccc7c6)cc54)c32)cc1 ; c1ccc(-n2c3ccccc3c3ccc(N(c4cccc5c4oc4c6ccccc6c(-c6ccc7ccccc7c6)cc54)c4cccc5c4oc4c6ccccc6c(-c6ccc7ccccc7c6)cc54)cc32)cc1]

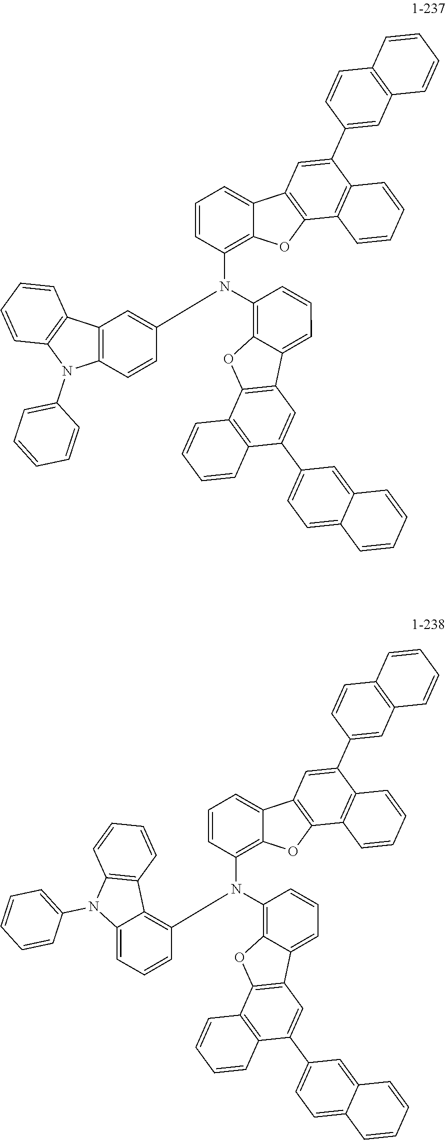 [SMILES: c1ccc(-n2c3ccccc3c3cc(N(c4cccc5c4oc4c6ccccc6c(-c6ccc7ccccc7c6)cc54)c4cccc5c4oc4c6ccccc6c(-c6ccc7ccccc7c6)cc54)ccc32)cc1 ; c1ccc(-n2c3ccccc3c3c(N(c4cccc5c4oc4c6ccccc6c(-c6ccc7ccccc7c6)cc54)c4cccc5c4oc4c6ccccc6c(-c6ccc7ccccc7c6)cc54)cccc32)cc1]

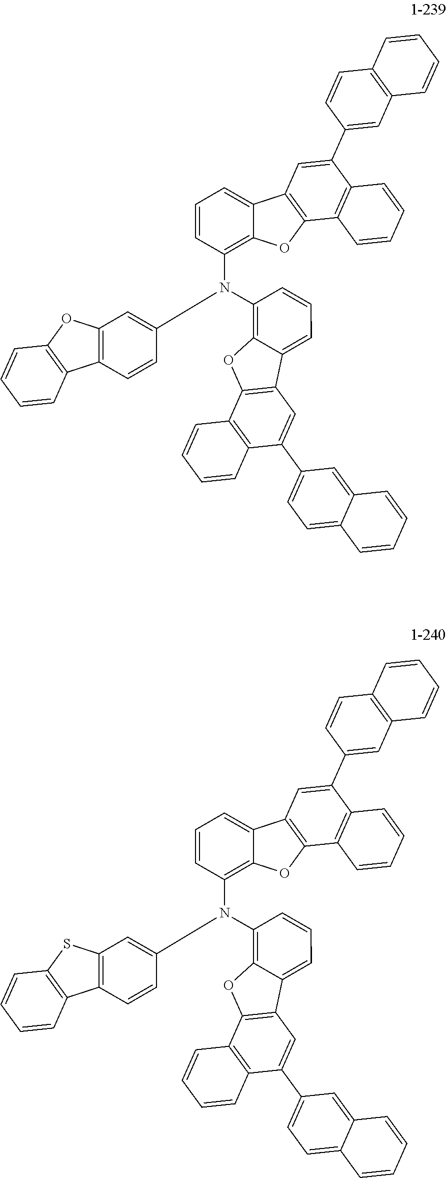 [SMILES: c1ccc2cc(-c3cc4c5cccc(N(c6ccc7c(c6)oc6ccccc67)c6cccc7c6oc6c8ccccc8c(-c8ccc9ccccc9c8)cc76)c5oc4c4ccccc34)ccc2c1 ; c1ccc2cc(-c3cc4c5cccc(N(c6ccc7c(c6)sc6ccccc67)c6cccc7c6oc6c8ccccc8c(-c8ccc9ccccc9c8)cc76)c5oc4c4ccccc34)ccc2c1]

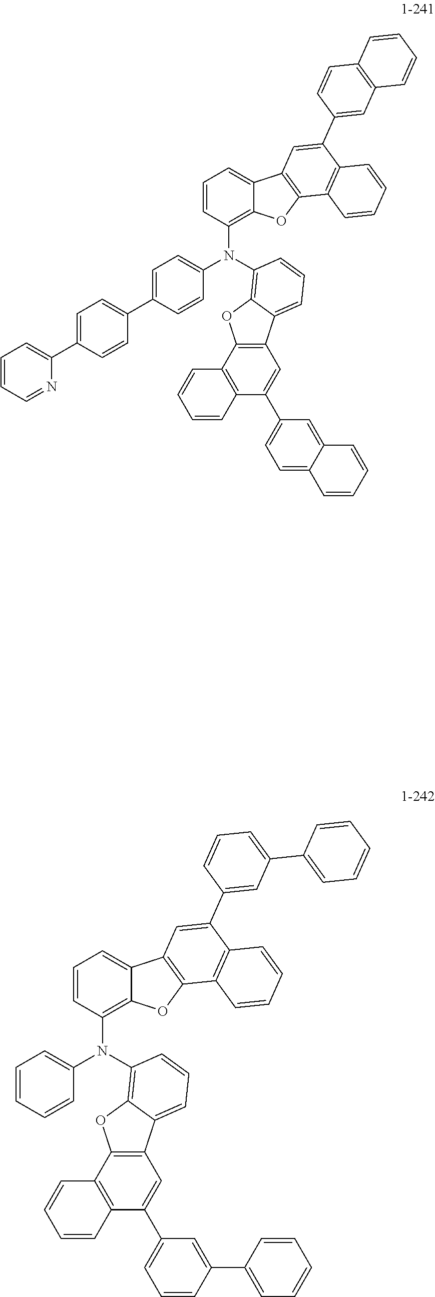 [SMILES: c1ccc(-c2ccc(-c3ccc(N(c4cccc5c4oc4c6ccccc6c(-c6ccc7ccccc7c6)cc54)c4cccc5c4oc4c6ccccc6c(-c6ccc7ccccc7c6)cc54)cc3)cc2)nc1 ; c1ccc(-c2cccc(-c3cc4c5cccc(N(c6ccccc6)c6cccc7c6oc6c8ccccc8c(-c8cccc(-c9ccccc9)c8)cc76)c5oc4c4ccccc34)c2)cc1]

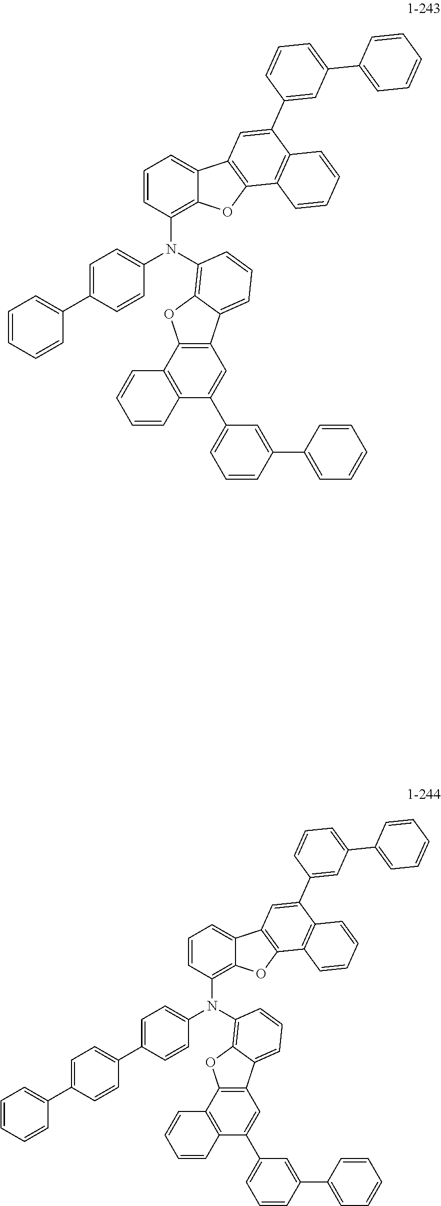 [SMILES: c1ccc(-c2ccc(N(c3cccc4c3oc3c5ccccc5c(-c5cccc(-c6ccccc6)c5)cc43)c3cccc4c3oc3c5ccccc5c(-c5cccc(-c6ccccc6)c5)cc43)cc2)cc1 ; c1ccc(-c2ccc(-c3ccc(N(c4cccc5c4oc4c6ccccc6c(-c6cccc(-c7ccccc7)c6)cc54)c4cccc5c4oc4c6ccccc6c(-c6cccc(-c7ccccc7)c6)cc54)cc3)cc2)cc1]

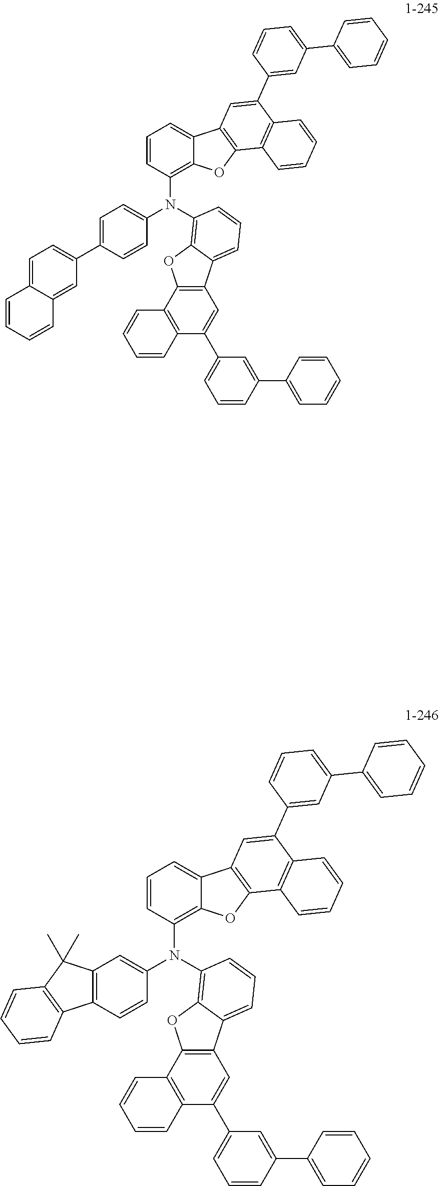 [SMILES: c1ccc(-c2cccc(-c3cc4c5cccc(N(c6ccc(-c7ccc8ccccc8c7)cc6)c6cccc7c6oc6c8ccccc8c(-c8cccc(-c9ccccc9)c8)cc76)c5oc4c4ccccc34)c2)cc1 ; CC1(C)c2ccccc2-c2ccc(N(c3cccc4c3oc3c5ccccc5c(-c5cccc(-c6ccccc6)c5)cc43)c3cccc4c3oc3c5ccccc5c(-c5cccc(-c6ccccc6)c5)cc43)cc21]

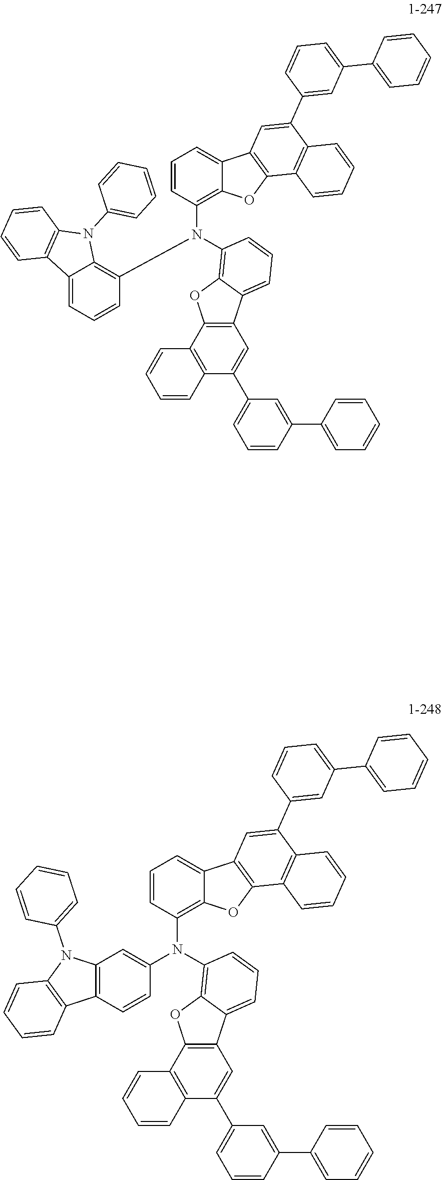 [SMILES: c1ccc(-c2cccc(-c3cc4c5cccc(N(c6cccc7c6oc6c8ccccc8c(-c8cccc(-c9ccccc9)c8)cc76)c6cccc7c8ccccc8n(-c8ccccc8)c67)c5oc4c4ccccc34)c2)cc1 ; c1ccc(-c2cccc(-c3cc4c5cccc(N(c6ccc7c8ccccc8n(-c8ccccc8)c7c6)c6cccc7c6oc6c8ccccc8c(-c8cccc(-c9ccccc9)c8)cc76)c5oc4c4ccccc34)c2)cc1]

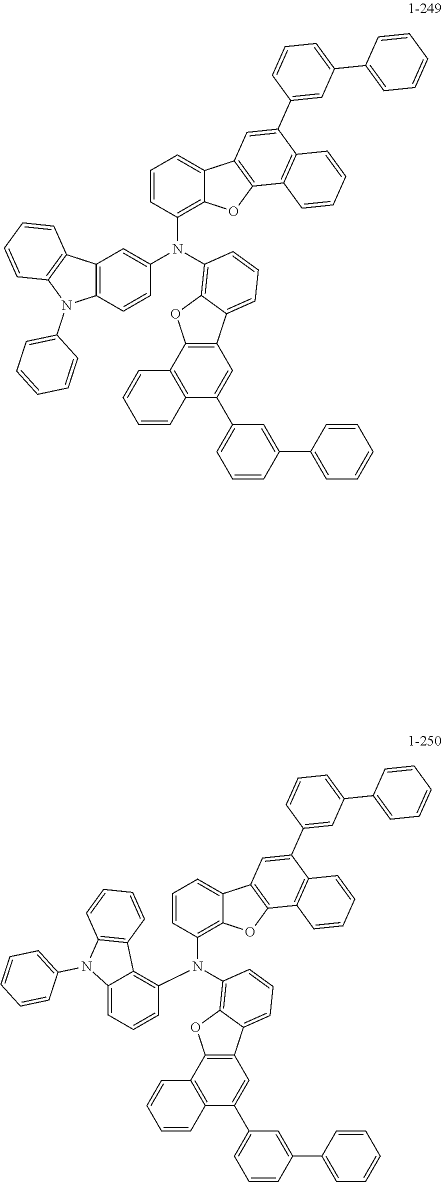 [SMILES: c1ccc(-c2cccc(-c3cc4c5cccc(N(c6ccc7c(c6)c6ccccc6n7-c6ccccc6)c6cccc7c6oc6c8ccccc8c(-c8cccc(-c9ccccc9)c8)cc76)c5oc4c4ccccc34)c2)cc1 ; c1ccc(-c2cccc(-c3cc4c5cccc(N(c6cccc7c6oc6c8ccccc8c(-c8cccc(-c9ccccc9)c8)cc76)c6cccc7c6c6ccccc6n7-c6ccccc6)c5oc4c4ccccc34)c2)cc1]

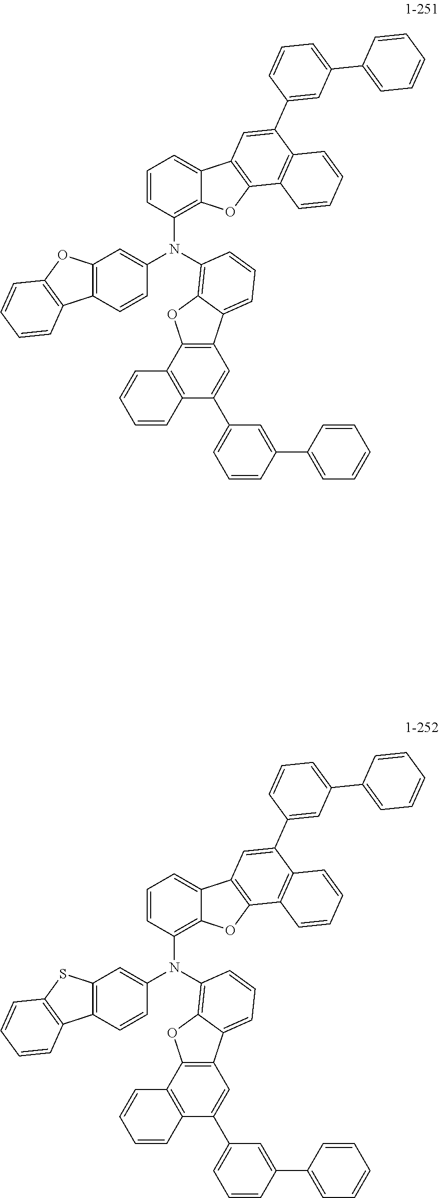 [SMILES: c1ccc(-c2cccc(-c3cc4c5cccc(N(c6ccc7c(c6)oc6ccccc67)c6cccc7c6oc6c8ccccc8c(-c8cccc(-c9ccccc9)c8)cc76)c5oc4c4ccccc34)c2)cc1 ; c1ccc(-c2cccc(-c3cc4c5cccc(N(c6ccc7c(c6)sc6ccccc67)c6cccc7c6oc6c8ccccc8c(-c8cccc(-c9ccccc9)c8)cc76)c5oc4c4ccccc34)c2)cc1]

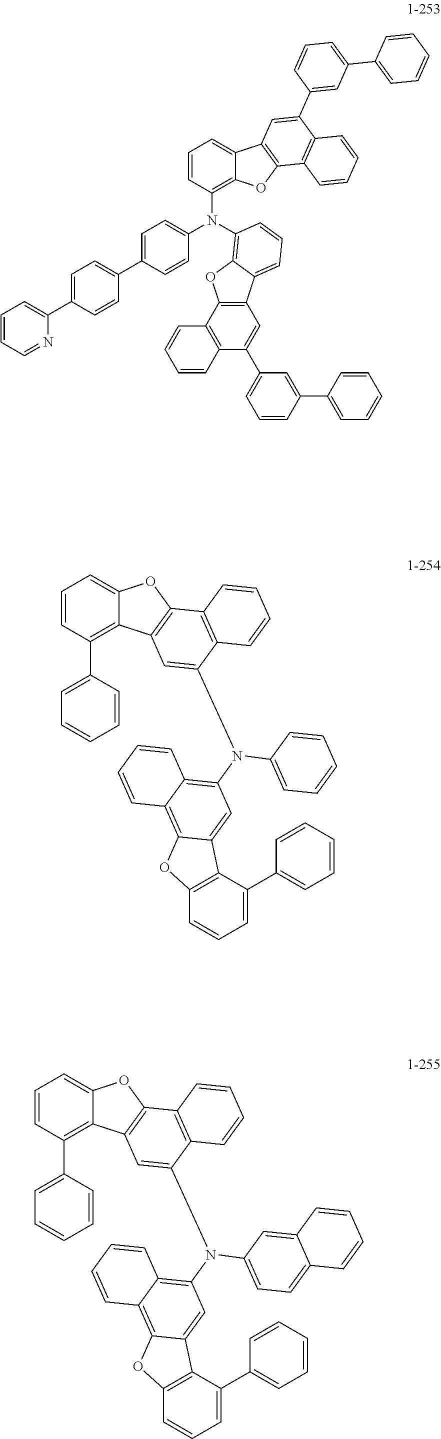 [SMILES: c1ccc(-c2cccc(-c3cc4c5cccc(N(c6ccc(-c7ccc(-c8ccccn8)cc7)cc6)c6cccc7c6oc6c8ccccc8c(-c8cccc(-c9ccccc9)c8)cc76)c5oc4c4ccccc34)c2)cc1 ; c1ccc(-c2cccc3oc4c5ccccc5c(N(c5ccccc5)c5cc6c(oc7cccc(-c8ccccc8)c76)c6ccccc56)cc4c23)cc1 ; c1ccc(-c2cccc3oc4c5ccccc5c(N(c5ccc6ccccc6c5)c5cc6c(oc7cccc(-c8ccccc8)c76)c6ccccc56)cc4c23)cc1]

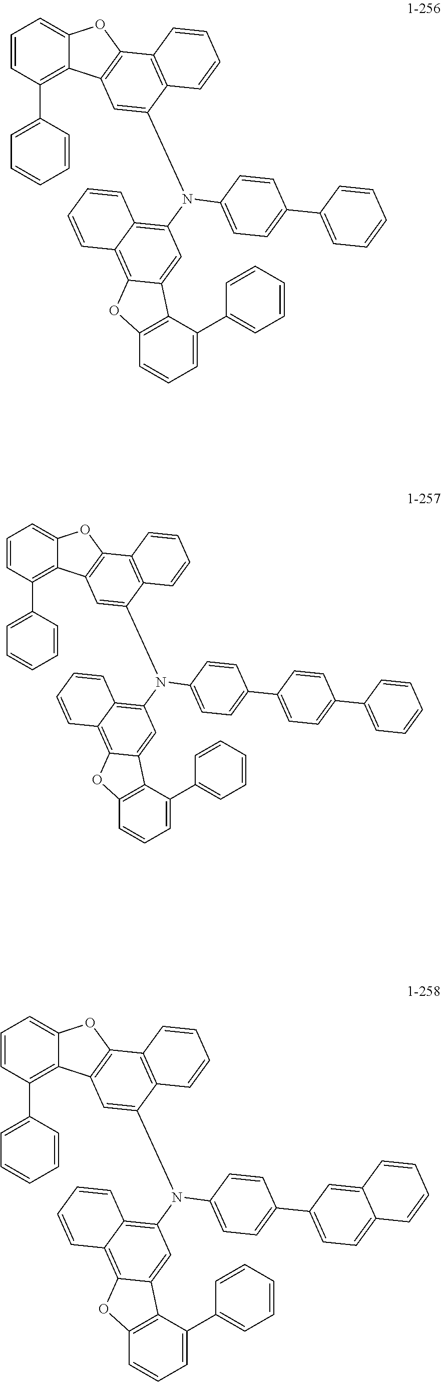 [SMILES: c1ccc(-c2ccc(N(c3cc4c(oc5cccc(-c6ccccc6)c54)c4ccccc34)c3cc4c(oc5cccc(-c6ccccc6)c54)c4ccccc34)cc2)cc1 ; c1ccc(-c2ccc(-c3ccc(N(c4cc5c(oc6cccc(-c7ccccc7)c65)c5ccccc45)c4cc5c(oc6cccc(-c7ccccc7)c65)c5ccccc45)cc3)cc2)cc1 ; c1ccc(-c2cccc3oc4c5ccccc5c(N(c5ccc(-c6ccc7ccccc7c6)cc5)c5cc6c(oc7cccc(-c8ccccc8)c76)c6ccccc56)cc4c23)cc1]

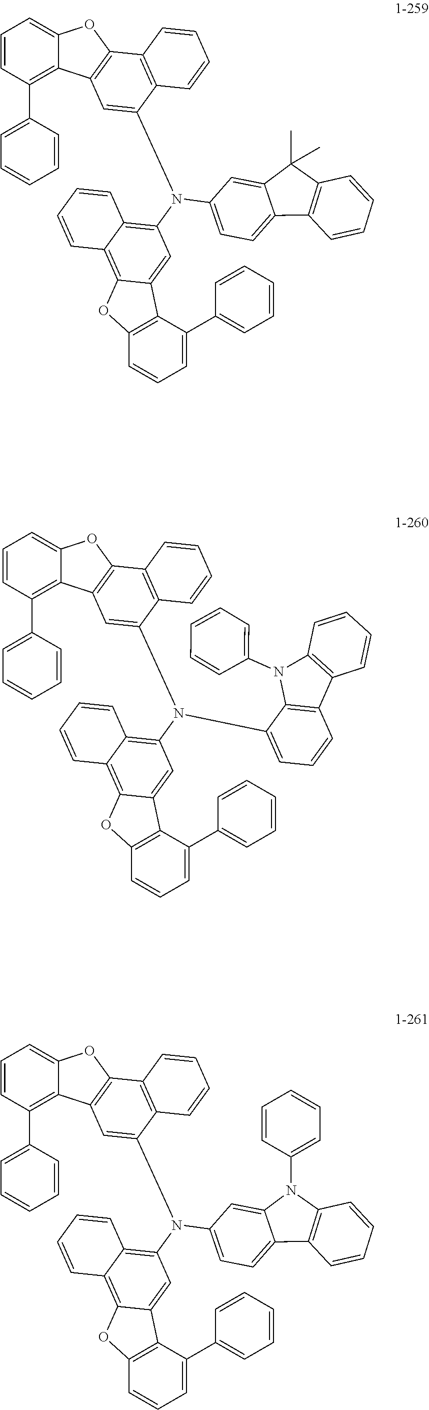 [SMILES: CC1(C)c2ccccc2-c2ccc(N(c3cc4c(oc5cccc(-c6ccccc6)c54)c4ccccc34)c3cc4c(oc5cccc(-c6ccccc6)c54)c4ccccc34)cc21 ; c1ccc(-c2cccc3oc4c5ccccc5c(N(c5cc6c(oc7cccc(-c8ccccc8)c76)c6ccccc56)c5cccc6c7ccccc7n(-c7ccccc7)c56)cc4c23)cc1 ; c1ccc(-c2cccc3oc4c5ccccc5c(N(c5ccc6c7ccccc7n(-c7ccccc7)c6c5)c5cc6c(oc7cccc(-c8ccccc8)c76)c6ccccc56)cc4c23)cc1]

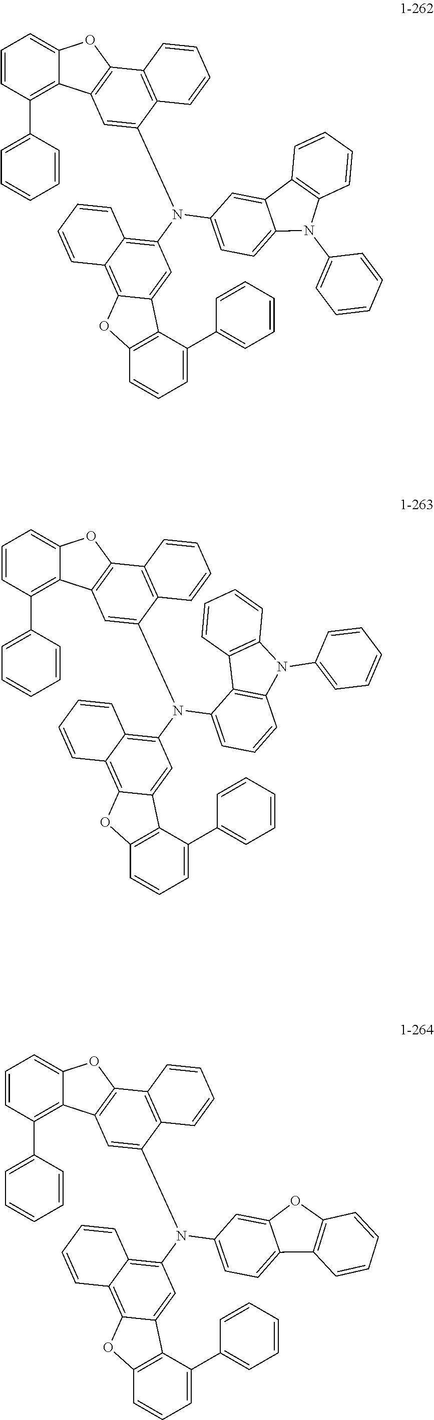 [SMILES: c1ccc(-c2cccc3oc4c5ccccc5c(N(c5ccc6c(c5)c5ccccc5n6-c5ccccc5)c5cc6c(oc7cccc(-c8ccccc8)c76)c6ccccc56)cc4c23)cc1 ; c1ccc(-c2cccc3oc4c5ccccc5c(N(c5cc6c(oc7cccc(-c8ccccc8)c76)c6ccccc56)c5cccc6c5c5ccccc5n6-c5ccccc5)cc4c23)cc1 ; c1ccc(-c2cccc3oc4c5ccccc5c(N(c5ccc6c(c5)oc5ccccc56)c5cc6c(oc7cccc(-c8ccccc8)c76)c6ccccc56)cc4c23)cc1]

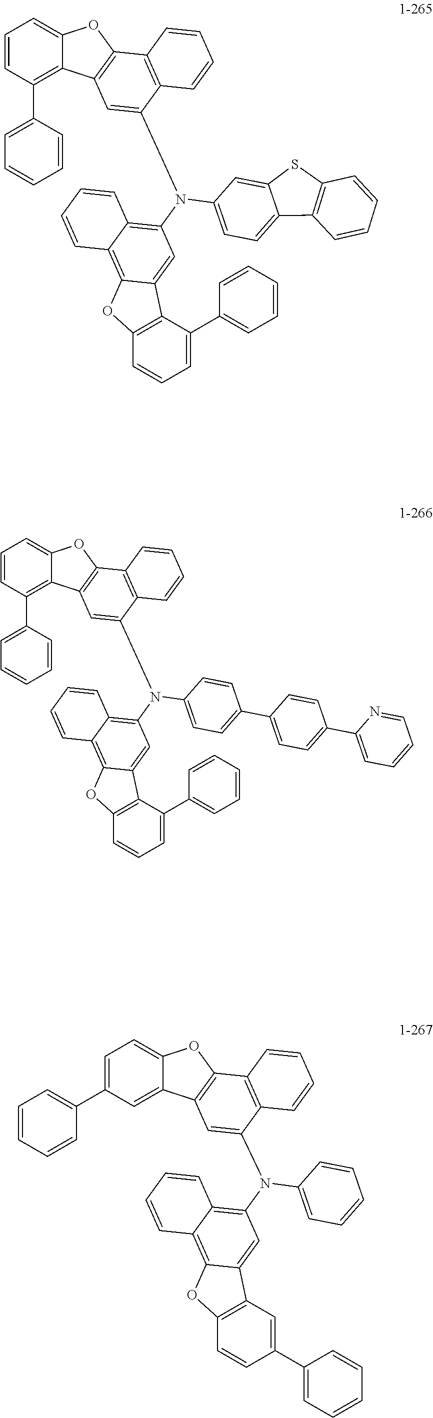 [SMILES: c1ccc(-c2cccc3oc4c5ccccc5c(N(c5ccc6c(c5)sc5ccccc56)c5cc6c(oc7cccc(-c8ccccc8)c76)c6ccccc56)cc4c23)cc1 ; c1ccc(-c2cccc3oc4c5ccccc5c(N(c5ccc(-c6ccc(-c7ccccn7)cc6)cc5)c5cc6c(oc7cccc(-c8ccccc8)c76)c6ccccc56)cc4c23)cc1 ; c1ccc(-c2ccc3oc4c5ccccc5c(N(c5ccccc5)c5cc6c7cc(-c8ccccc8)ccc7oc6c6ccccc56)cc4c3c2)cc1]

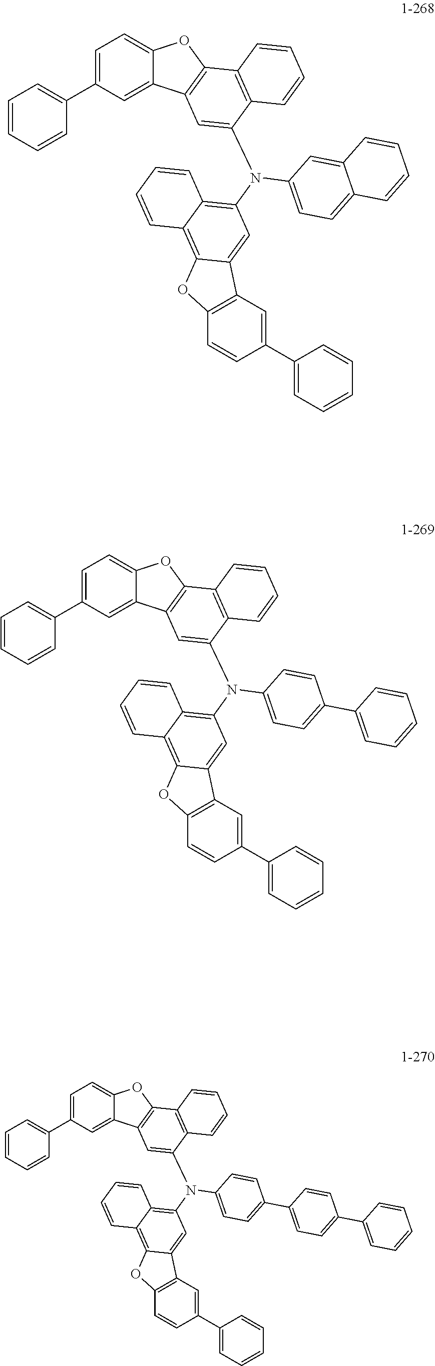 [SMILES: c1ccc(-c2ccc3oc4c5ccccc5c(N(c5ccc6ccccc6c5)c5cc6c7cc(-c8ccccc8)ccc7oc6c6ccccc56)cc4c3c2)cc1 ; c1ccc(-c2ccc(N(c3cc4c5cc(-c6ccccc6)ccc5oc4c4ccccc34)c3cc4c5cc(-c6ccccc6)ccc5oc4c4ccccc34)cc2)cc1 ; c1ccc(-c2ccc(-c3ccc(N(c4cc5c6cc(-c7ccccc7)ccc6oc5c5ccccc45)c4cc5c6cc(-c7ccccc7)ccc6oc5c5ccccc45)cc3)cc2)cc1]

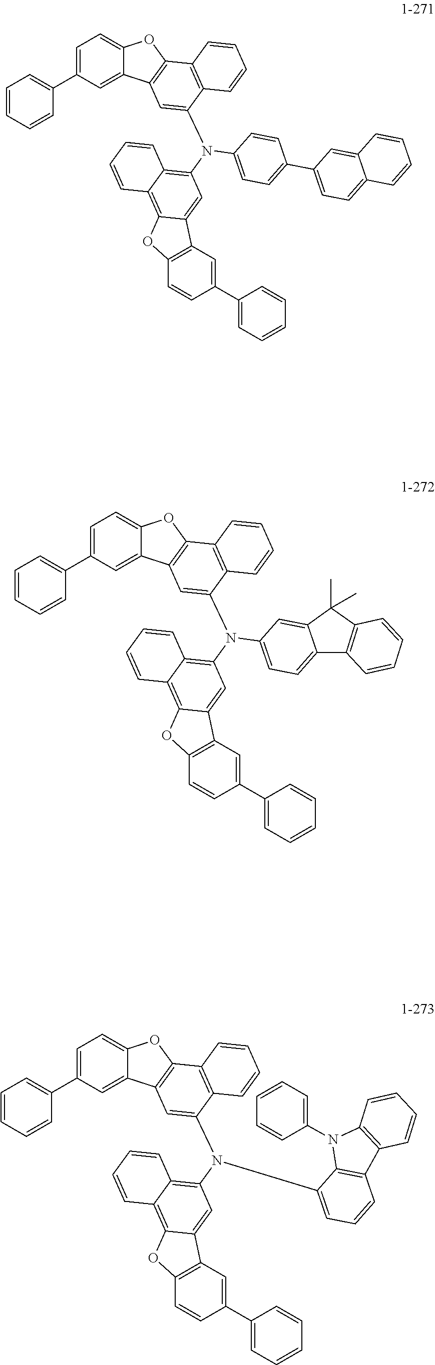 [SMILES: c1ccc(-c2ccc3oc4c5ccccc5c(N(c5ccc(-c6ccc7ccccc7c6)cc5)c5cc6c7cc(-c8ccccc8)ccc7oc6c6ccccc56)cc4c3c2)cc1 ; CC1(C)c2ccccc2-c2ccc(N(c3cc4c5cc(-c6ccccc6)ccc5oc4c4ccccc34)c3cc4c5cc(-c6ccccc6)ccc5oc4c4ccccc34)cc21 ; c1ccc(-c2ccc3oc4c5ccccc5c(N(c5cc6c7cc(-c8ccccc8)ccc7oc6c6ccccc56)c5cccc6c7ccccc7n(-c7ccccc7)c56)cc4c3c2)cc1]

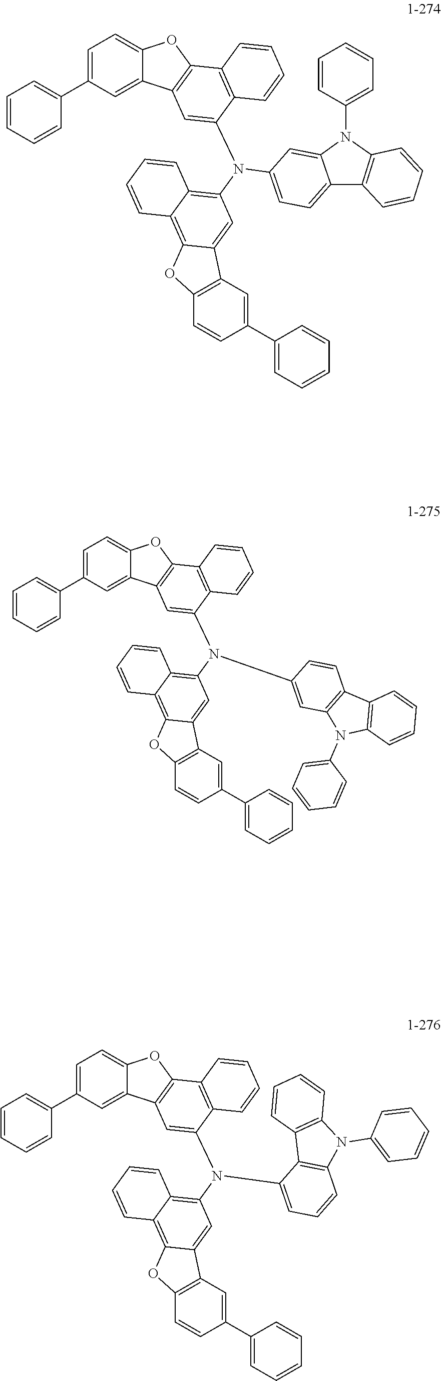 [SMILES: c1ccc(-c2ccc3oc4c5ccccc5c(N(c5ccc6c7ccccc7n(-c7ccccc7)c6c5)c5cc6c7cc(-c8ccccc8)ccc7oc6c6ccccc56)cc4c3c2)cc1 ; c1ccc(-c2ccc3oc4c5ccccc5c(N(c5ccc6c7ccccc7n(-c7ccccc7)c6c5)c5cc6c7cc(-c8ccccc8)ccc7oc6c6ccccc56)cc4c3c2)cc1 ; c1ccc(-c2ccc3oc4c5ccccc5c(N(c5cc6c7cc(-c8ccccc8)ccc7oc6c6ccccc56)c5cccc6c5c5ccccc5n6-c5ccccc5)cc4c3c2)cc1]

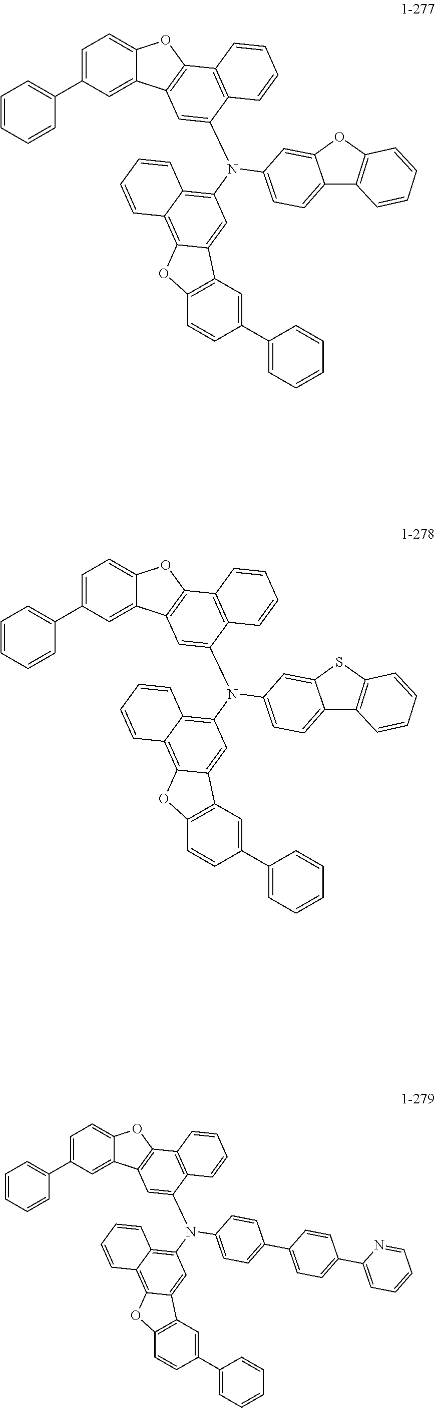 [SMILES: c1ccc(-c2ccc3oc4c5ccccc5c(N(c5ccc6c(c5)oc5ccccc56)c5cc6c7cc(-c8ccccc8)ccc7oc6c6ccccc56)cc4c3c2)cc1 ; c1ccc(-c2ccc3oc4c5ccccc5c(N(c5ccc6c(c5)sc5ccccc56)c5cc6c7cc(-c8ccccc8)ccc7oc6c6ccccc56)cc4c3c2)cc1 ; c1ccc(-c2ccc3oc4c5ccccc5c(N(c5ccc(-c6ccc(-c7ccccn7)cc6)cc5)c5cc6c7cc(-c8ccccc8)ccc7oc6c6ccccc56)cc4c3c2)cc1]

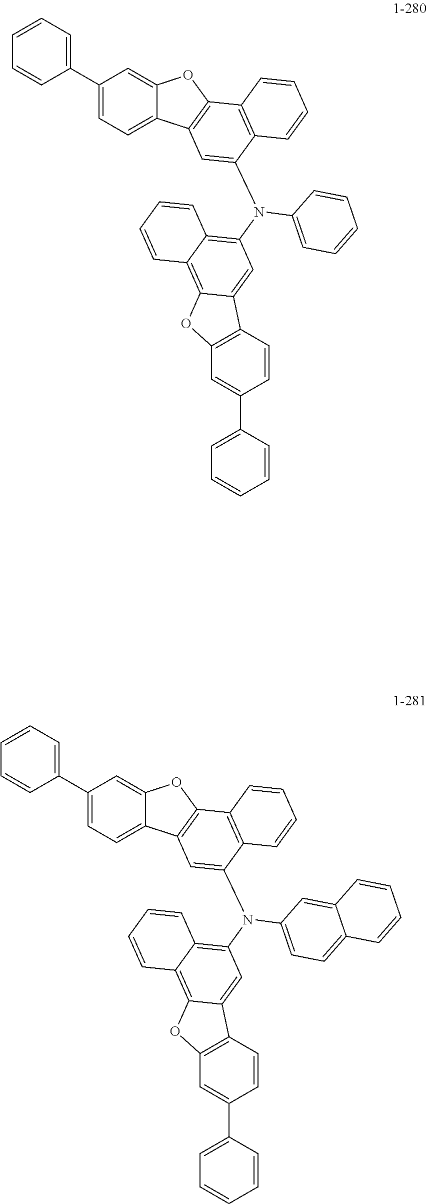 [SMILES: c1ccc(-c2ccc3c(c2)oc2c4ccccc4c(N(c4ccccc4)c4cc5c6ccc(-c7ccccc7)cc6oc5c5ccccc45)cc32)cc1 ; c1ccc(-c2ccc3c(c2)oc2c4ccccc4c(N(c4ccc5ccccc5c4)c4cc5c6ccc(-c7ccccc7)cc6oc5c5ccccc45)cc32)cc1]

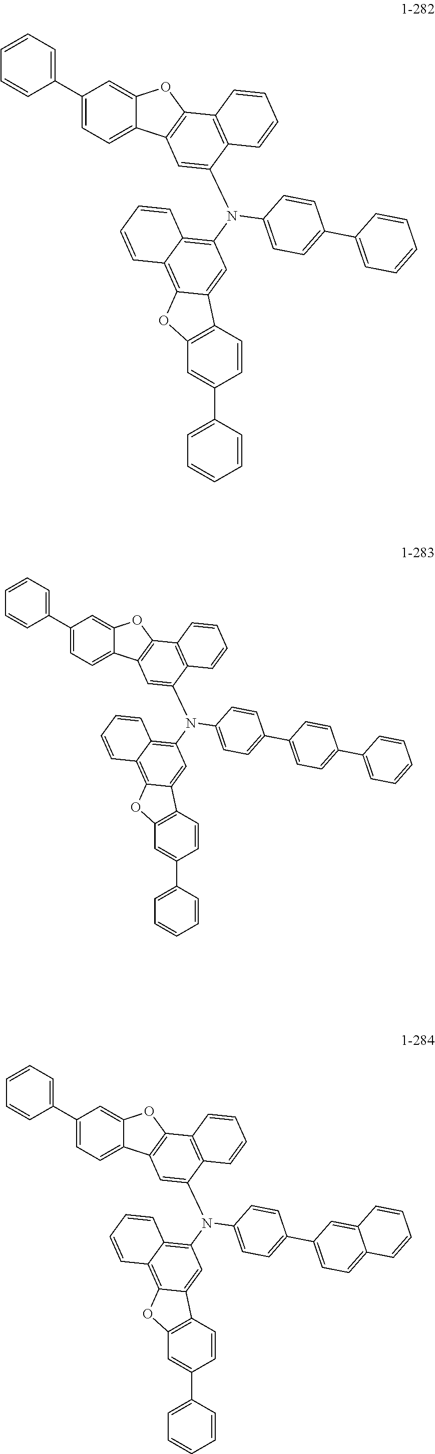 [SMILES: c1ccc(-c2ccc(N(c3cc4c5ccc(-c6ccccc6)cc5oc4c4ccccc34)c3cc4c5ccc(-c6ccccc6)cc5oc4c4ccccc34)cc2)cc1 ; c1ccc(-c2ccc(-c3ccc(N(c4cc5c6ccc(-c7ccccc7)cc6oc5c5ccccc45)c4cc5c6ccc(-c7ccccc7)cc6oc5c5ccccc45)cc3)cc2)cc1 ; c1ccc(-c2ccc3c(c2)oc2c4ccccc4c(N(c4ccc(-c5ccc6ccccc6c5)cc4)c4cc5c6ccc(-c7ccccc7)cc6oc5c5ccccc45)cc32)cc1]

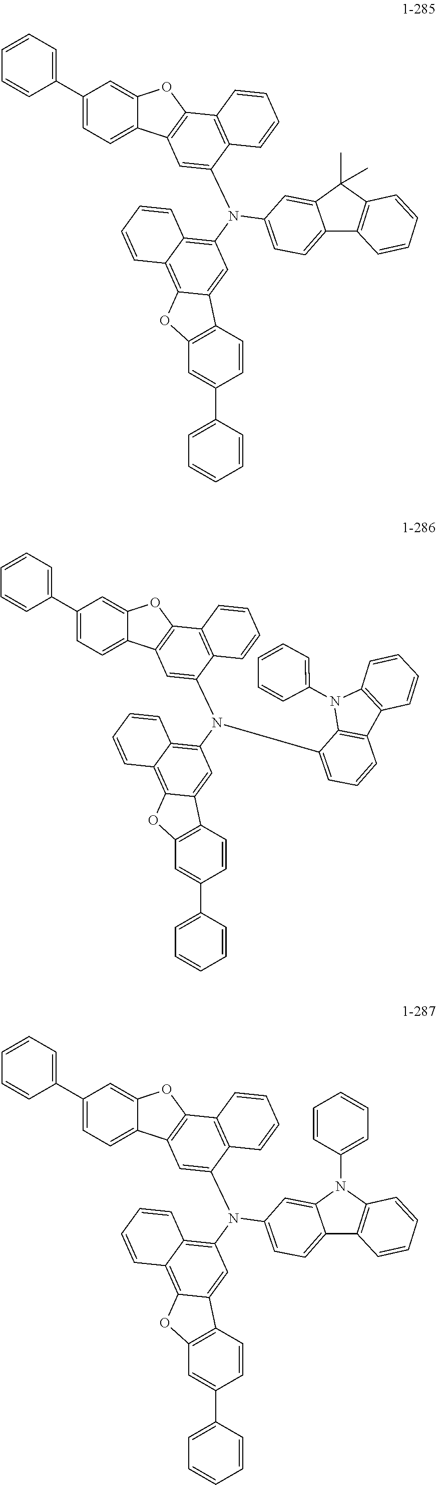 [SMILES: CC1(C)c2ccccc2-c2ccc(N(c3cc4c5ccc(-c6ccccc6)cc5oc4c4ccccc34)c3cc4c5ccc(-c6ccccc6)cc5oc4c4ccccc34)cc21 ; c1ccc(-c2ccc3c(c2)oc2c4ccccc4c(N(c4cc5c6ccc(-c7ccccc7)cc6oc5c5ccccc45)c4cccc5c6ccccc6n(-c6ccccc6)c45)cc32)cc1 ; c1ccc(-c2ccc3c(c2)oc2c4ccccc4c(N(c4ccc5c6ccccc6n(-c6ccccc6)c5c4)c4cc5c6ccc(-c7ccccc7)cc6oc5c5ccccc45)cc32)cc1]

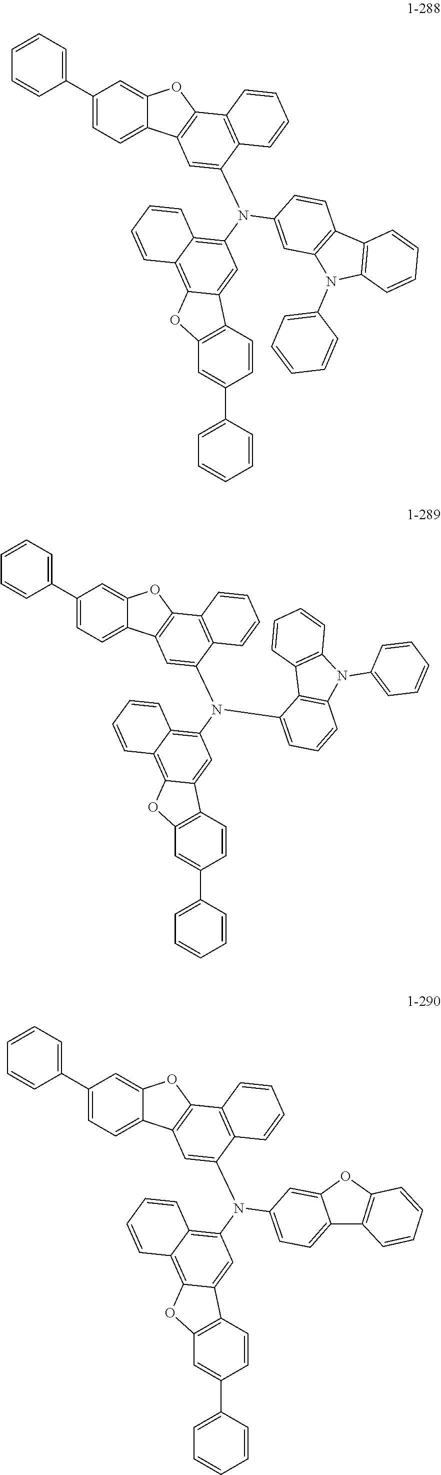 [SMILES: c1ccc(-c2ccc3c(c2)oc2c4ccccc4c(N(c4ccc5c6ccccc6n(-c6ccccc6)c5c4)c4cc5c6ccc(-c7ccccc7)cc6oc5c5ccccc45)cc32)cc1 ; c1ccc(-c2ccc3c(c2)oc2c4ccccc4c(N(c4cc5c6ccc(-c7ccccc7)cc6oc5c5ccccc45)c4cccc5c4c4ccccc4n5-c4ccccc4)cc32)cc1 ; c1ccc(-c2ccc3c(c2)oc2c4ccccc4c(N(c4ccc5c(c4)oc4ccccc45)c4cc5c6ccc(-c7ccccc7)cc6oc5c5ccccc45)cc32)cc1]

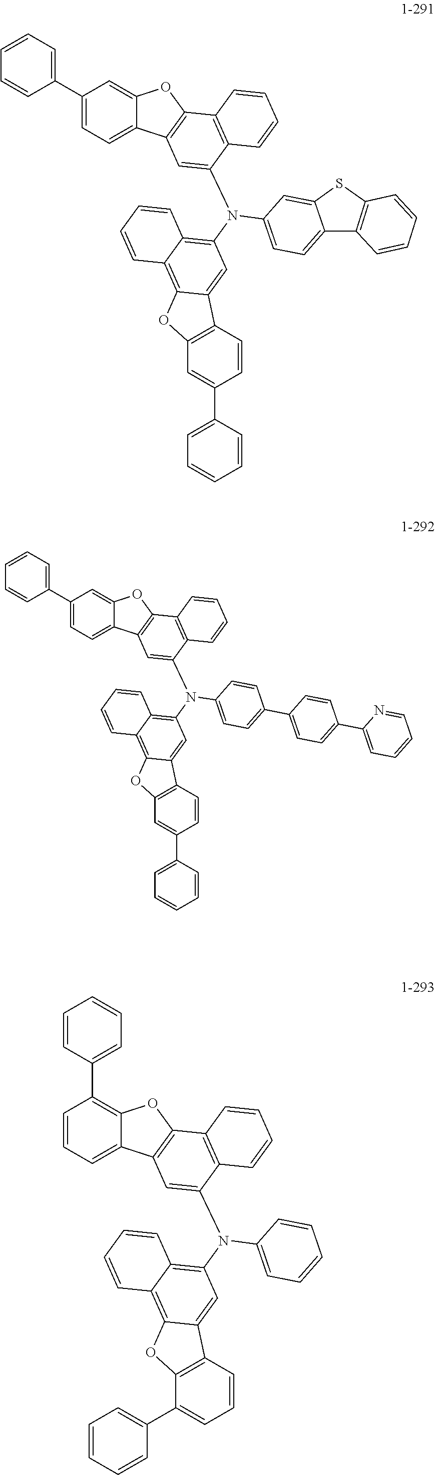 [SMILES: c1ccc(-c2ccc3c(c2)oc2c4ccccc4c(N(c4ccc5c(c4)sc4ccccc45)c4cc5c6ccc(-c7ccccc7)cc6oc5c5ccccc45)cc32)cc1 ; c1ccc(-c2ccc3c(c2)oc2c4ccccc4c(N(c4ccc(-c5ccc(-c6ccccn6)cc5)cc4)c4cc5c6ccc(-c7ccccc7)cc6oc5c5ccccc45)cc32)cc1 ; c1ccc(-c2cccc3c2oc2c4ccccc4c(N(c4ccccc4)c4cc5c6cccc(-c7ccccc7)c6oc5c5ccccc45)cc32)cc1]

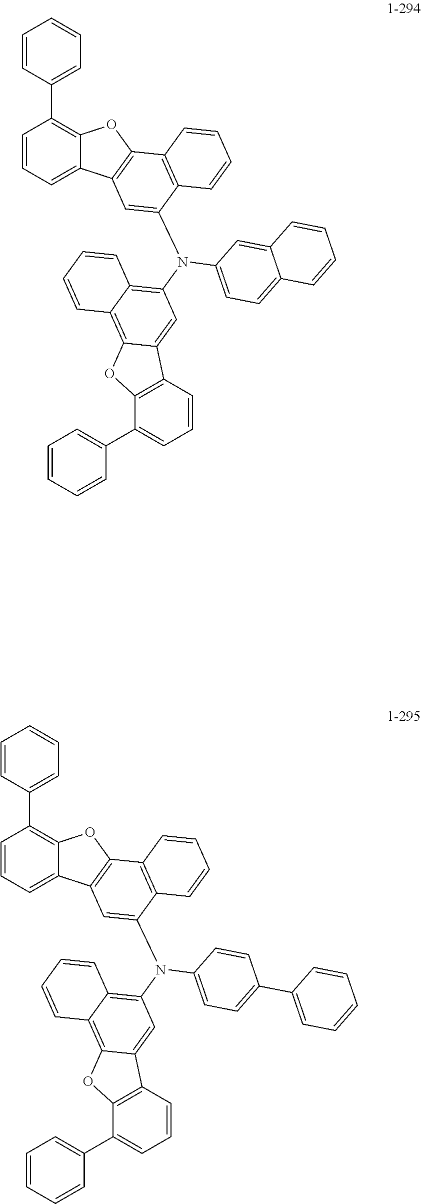 [SMILES: c1ccc(-c2cccc3c2oc2c4ccccc4c(N(c4ccc5ccccc5c4)c4cc5c6cccc(-c7ccccc7)c6oc5c5ccccc45)cc32)cc1 ; c1ccc(-c2ccc(N(c3cc4c5cccc(-c6ccccc6)c5oc4c4ccccc34)c3cc4c5cccc(-c6ccccc6)c5oc4c4ccccc34)cc2)cc1]

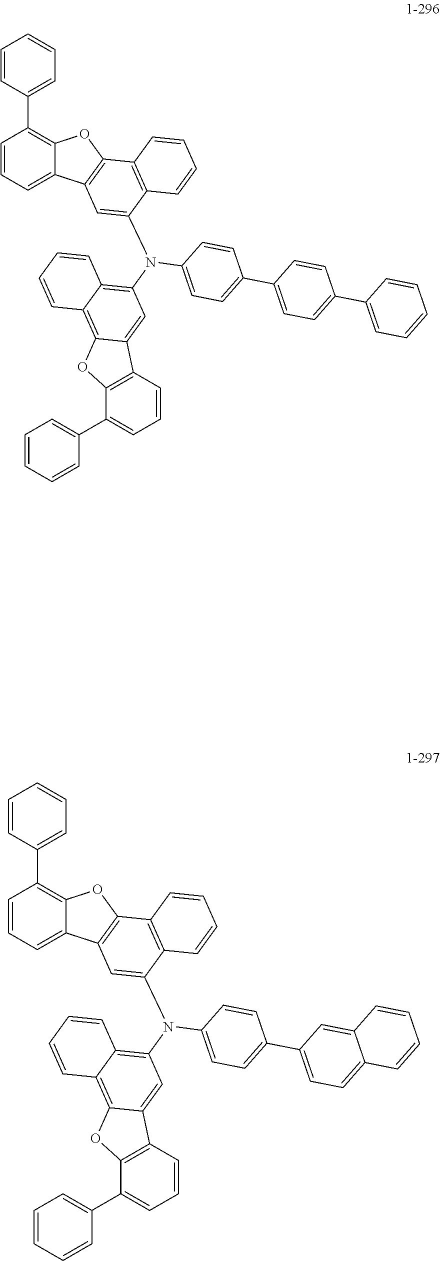 [SMILES: c1ccc(-c2ccc(-c3ccc(N(c4cc5c6cccc(-c7ccccc7)c6oc5c5ccccc45)c4cc5c6cccc(-c7ccccc7)c6oc5c5ccccc45)cc3)cc2)cc1 ; c1ccc(-c2cccc3c2oc2c4ccccc4c(N(c4ccc(-c5ccc6ccccc6c5)cc4)c4cc5c6cccc(-c7ccccc7)c6oc5c5ccccc45)cc32)cc1]

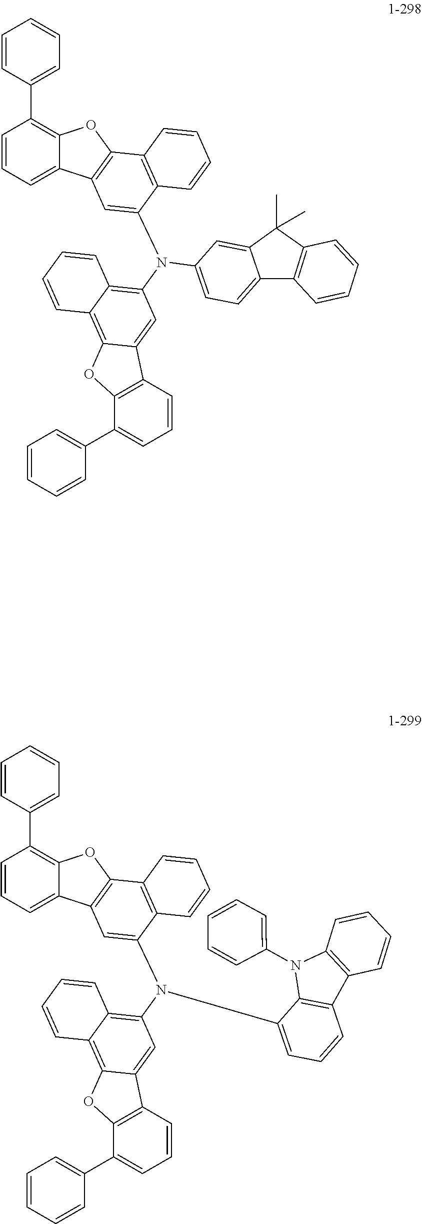 [SMILES: CC1(C)c2ccccc2-c2ccc(N(c3cc4c5cccc(-c6ccccc6)c5oc4c4ccccc34)c3cc4c5cccc(-c6ccccc6)c5oc4c4ccccc34)cc21 ; c1ccc(-c2cccc3c2oc2c4ccccc4c(N(c4cc5c6cccc(-c7ccccc7)c6oc5c5ccccc45)c4cccc5c6ccccc6n(-c6ccccc6)c45)cc32)cc1]

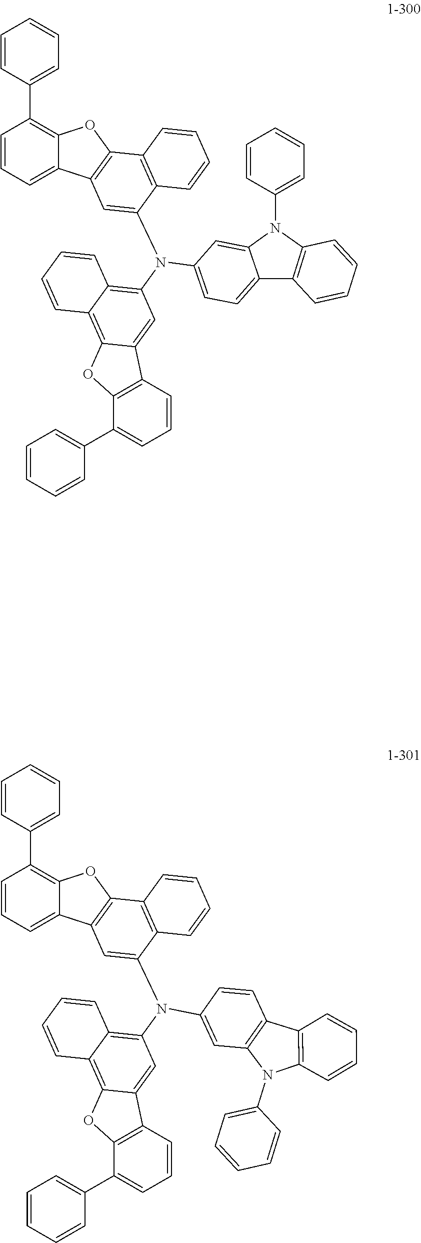 [SMILES: c1ccc(-c2cccc3c2oc2c4ccccc4c(N(c4ccc5c6ccccc6n(-c6ccccc6)c5c4)c4cc5c6cccc(-c7ccccc7)c6oc5c5ccccc45)cc32)cc1 ; c1ccc(-c2cccc3c2oc2c4ccccc4c(N(c4ccc5c6ccccc6n(-c6ccccc6)c5c4)c4cc5c6cccc(-c7ccccc7)c6oc5c5ccccc45)cc32)cc1]

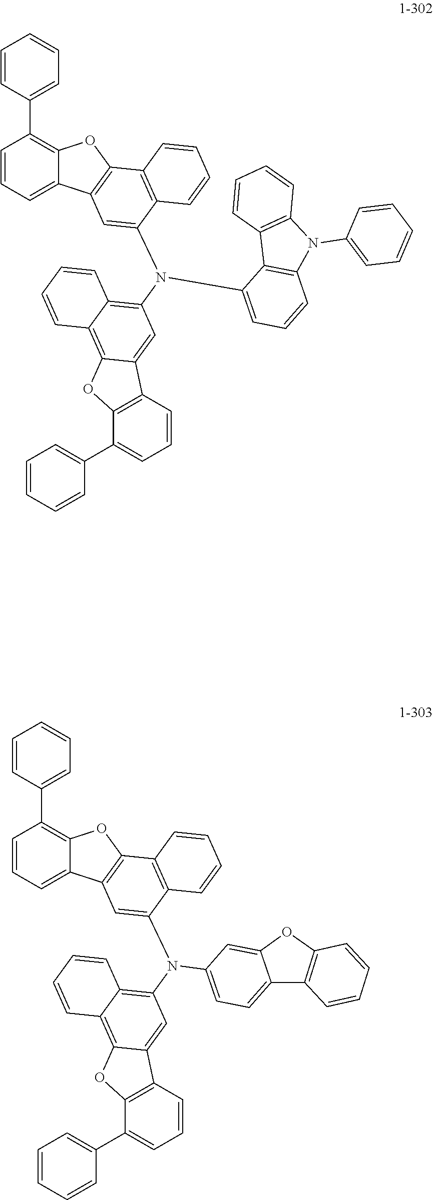 [SMILES: c1ccc(-c2cccc3c2oc2c4ccccc4c(N(c4cc5c6cccc(-c7ccccc7)c6oc5c5ccccc45)c4cccc5c4c4ccccc4n5-c4ccccc4)cc32)cc1 ; c1ccc(-c2cccc3c2oc2c4ccccc4c(N(c4ccc5c(c4)oc4ccccc45)c4cc5c6cccc(-c7ccccc7)c6oc5c5ccccc45)cc32)cc1]

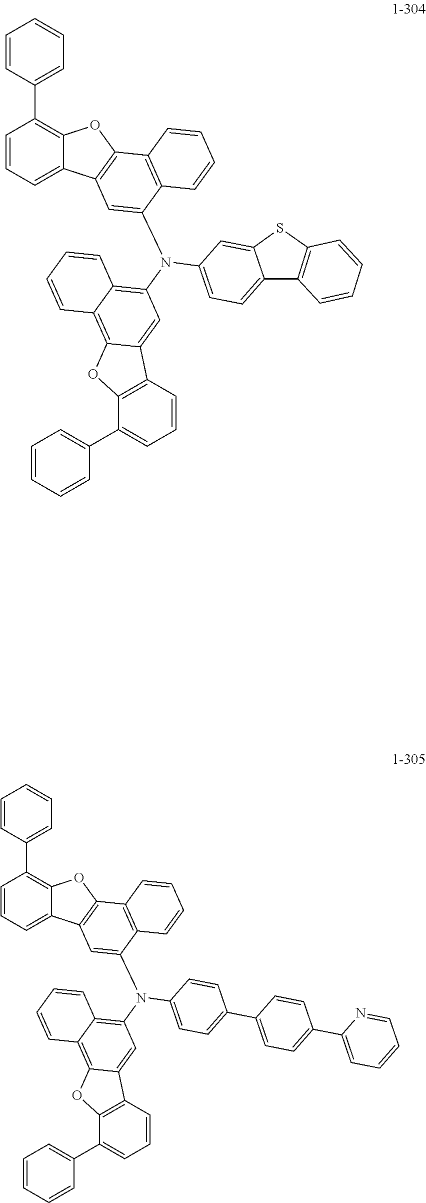 [SMILES: c1ccc(-c2cccc3c2oc2c4ccccc4c(N(c4ccc5c(c4)sc4ccccc45)c4cc5c6cccc(-c7ccccc7)c6oc5c5ccccc45)cc32)cc1 ; c1ccc(-c2cccc3c2oc2c4ccccc4c(N(c4ccc(-c5ccc(-c6ccccn6)cc5)cc4)c4cc5c6cccc(-c7ccccc7)c6oc5c5ccccc45)cc32)cc1]

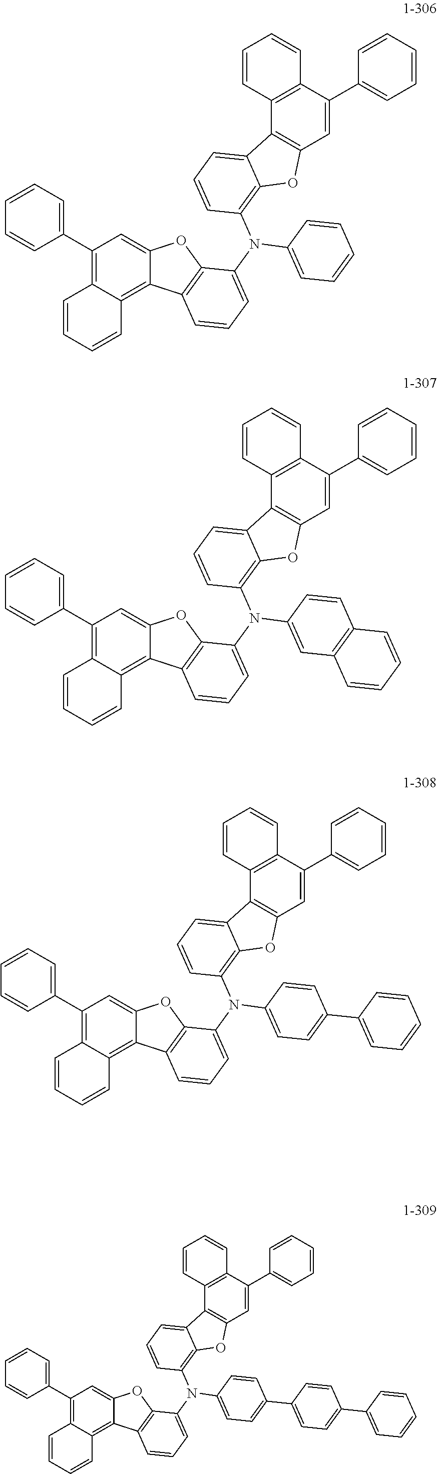 [SMILES: c1ccc(-c2cc3oc4c(N(c5ccccc5)c5cccc6c5oc5cc(-c7ccccc7)c7ccccc7c56)cccc4c3c3ccccc23)cc1 ; c1ccc(-c2cc3oc4c(N(c5ccc6ccccc6c5)c5cccc6c5oc5cc(-c7ccccc7)c7ccccc7c56)cccc4c3c3ccccc23)cc1 ; c1ccc(-c2ccc(N(c3cccc4c3oc3cc(-c5ccccc5)c5ccccc5c34)c3cccc4c3oc3cc(-c5ccccc5)c5ccccc5c34)cc2)cc1 ; c1ccc(-c2ccc(-c3ccc(N(c4cccc5c4oc4cc(-c6ccccc6)c6ccccc6c45)c4cccc5c4oc4cc(-c6ccccc6)c6ccccc6c45)cc3)cc2)cc1]

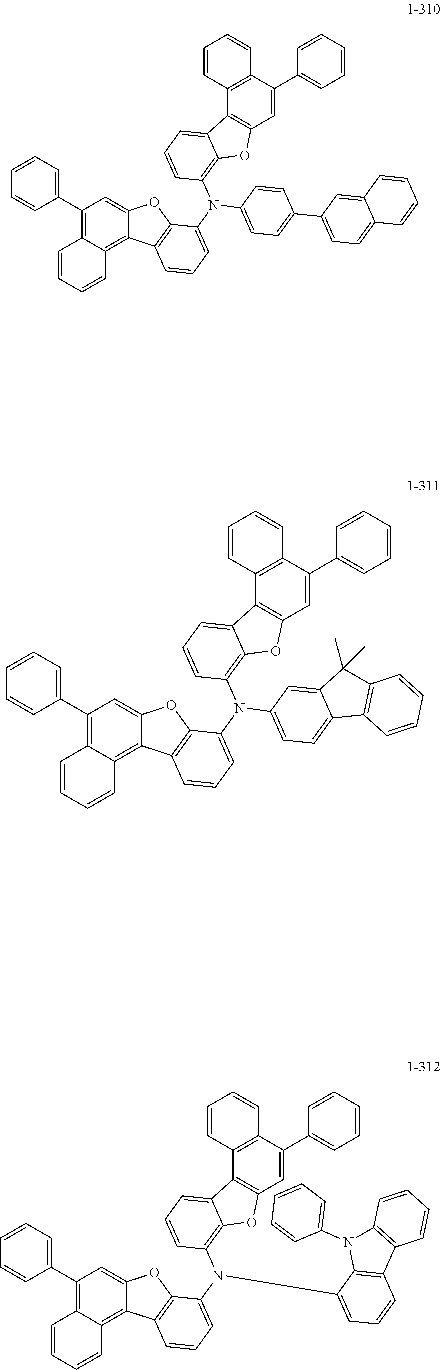 [SMILES: c1ccc(-c2cc3oc4c(N(c5ccc(-c6ccc7ccccc7c6)cc5)c5cccc6c5oc5cc(-c7ccccc7)c7ccccc7c56)cccc4c3c3ccccc23)cc1 ; CC1(C)c2ccccc2-c2ccc(N(c3cccc4c3oc3cc(-c5ccccc5)c5ccccc5c34)c3cccc4c3oc3cc(-c5ccccc5)c5ccccc5c34)cc21 ; c1ccc(-c2cc3oc4c(N(c5cccc6c5oc5cc(-c7ccccc7)c7ccccc7c56)c5cccc6c7ccccc7n(-c7ccccc7)c56)cccc4c3c3ccccc23)cc1]

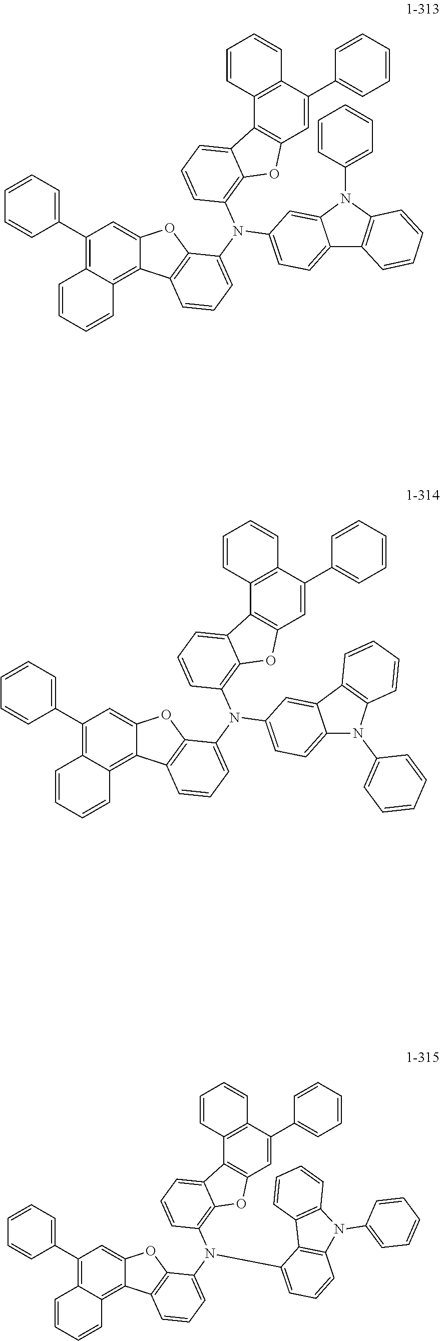 [SMILES: c1ccc(-c2cc3oc4c(N(c5ccc6c7ccccc7n(-c7ccccc7)c6c5)c5cccc6c5oc5cc(-c7ccccc7)c7ccccc7c56)cccc4c3c3ccccc23)cc1 ; c1ccc(-c2cc3oc4c(N(c5ccc6c(c5)c5ccccc5n6-c5ccccc5)c5cccc6c5oc5cc(-c7ccccc7)c7ccccc7c56)cccc4c3c3ccccc23)cc1 ; c1ccc(-c2cc3oc4c(N(c5cccc6c5oc5cc(-c7ccccc7)c7ccccc7c56)c5cccc6c5c5ccccc5n6-c5ccccc5)cccc4c3c3ccccc23)cc1]

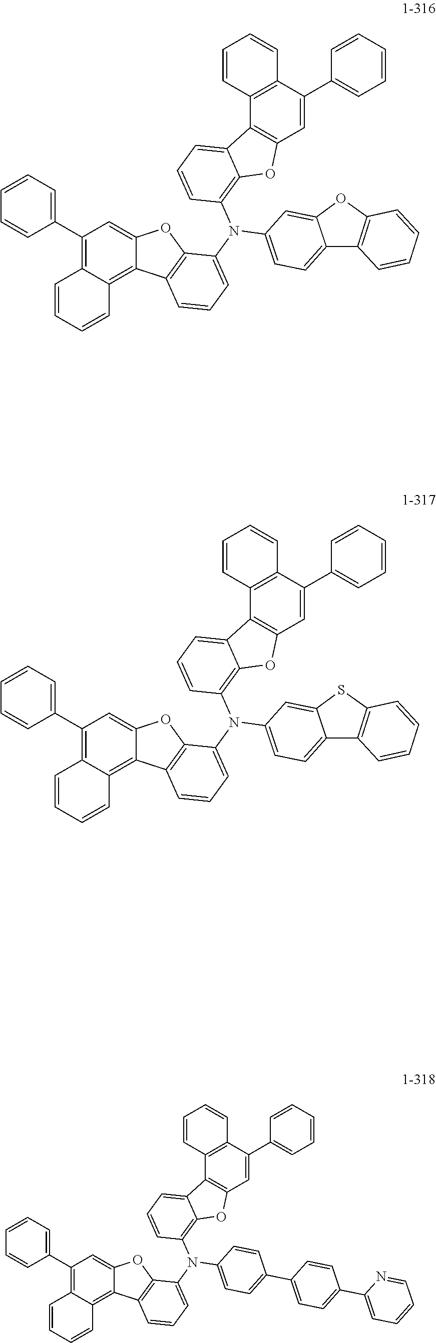 [SMILES: c1ccc(-c2cc3oc4c(N(c5ccc6c(c5)oc5ccccc56)c5cccc6c5oc5cc(-c7ccccc7)c7ccccc7c56)cccc4c3c3ccccc23)cc1 ; c1ccc(-c2cc3oc4c(N(c5ccc6c(c5)sc5ccccc56)c5cccc6c5oc5cc(-c7ccccc7)c7ccccc7c56)cccc4c3c3ccccc23)cc1 ; c1ccc(-c2cc3oc4c(N(c5ccc(-c6ccc(-c7ccccn7)cc6)cc5)c5cccc6c5oc5cc(-c7ccccc7)c7ccccc7c56)cccc4c3c3ccccc23)cc1]

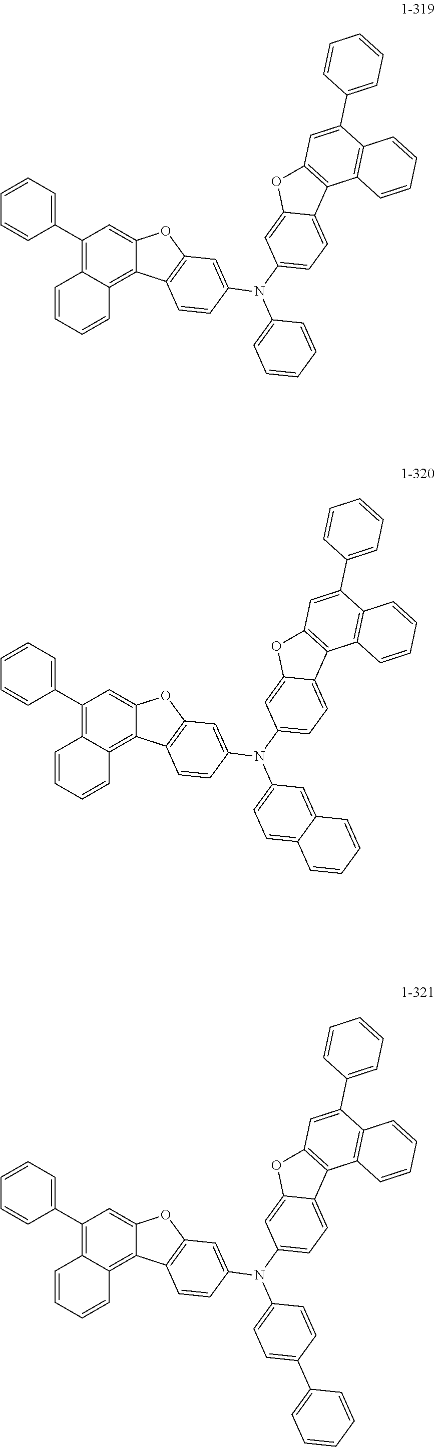 [SMILES: c1ccc(-c2cc3oc4cc(N(c5ccccc5)c5ccc6c(c5)oc5cc(-c7ccccc7)c7ccccc7c56)ccc4c3c3ccccc23)cc1 ; c1ccc(-c2cc3oc4cc(N(c5ccc6ccccc6c5)c5ccc6c(c5)oc5cc(-c7ccccc7)c7ccccc7c56)ccc4c3c3ccccc23)cc1 ; c1ccc(-c2ccc(N(c3ccc4c(c3)oc3cc(-c5ccccc5)c5ccccc5c34)c3ccc4c(c3)oc3cc(-c5ccccc5)c5ccccc5c34)cc2)cc1]

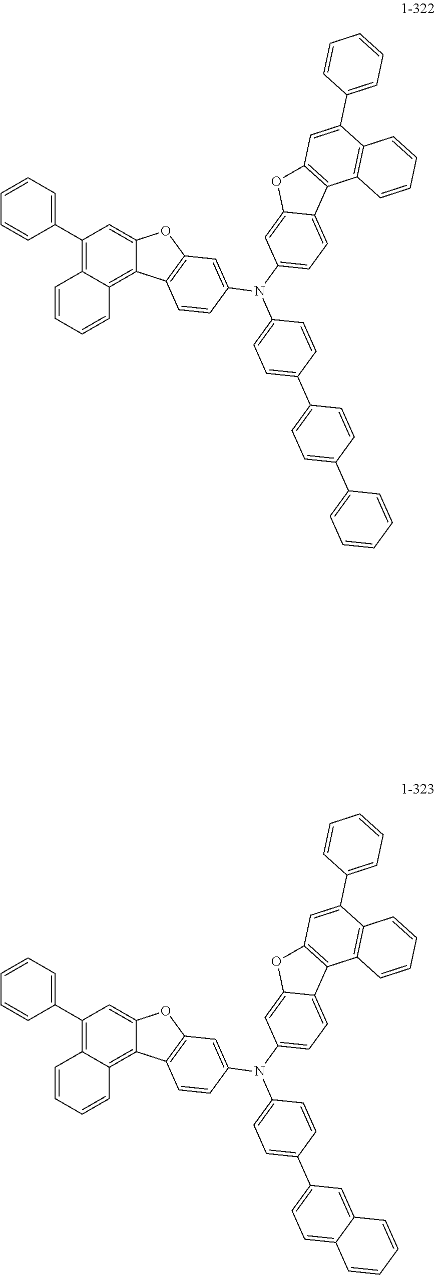 [SMILES: c1ccc(-c2ccc(-c3ccc(N(c4ccc5c(c4)oc4cc(-c6ccccc6)c6ccccc6c45)c4ccc5c(c4)oc4cc(-c6ccccc6)c6ccccc6c45)cc3)cc2)cc1 ; c1ccc(-c2cc3oc4cc(N(c5ccc(-c6ccc7ccccc7c6)cc5)c5ccc6c(c5)oc5cc(-c7ccccc7)c7ccccc7c56)ccc4c3c3ccccc23)cc1]

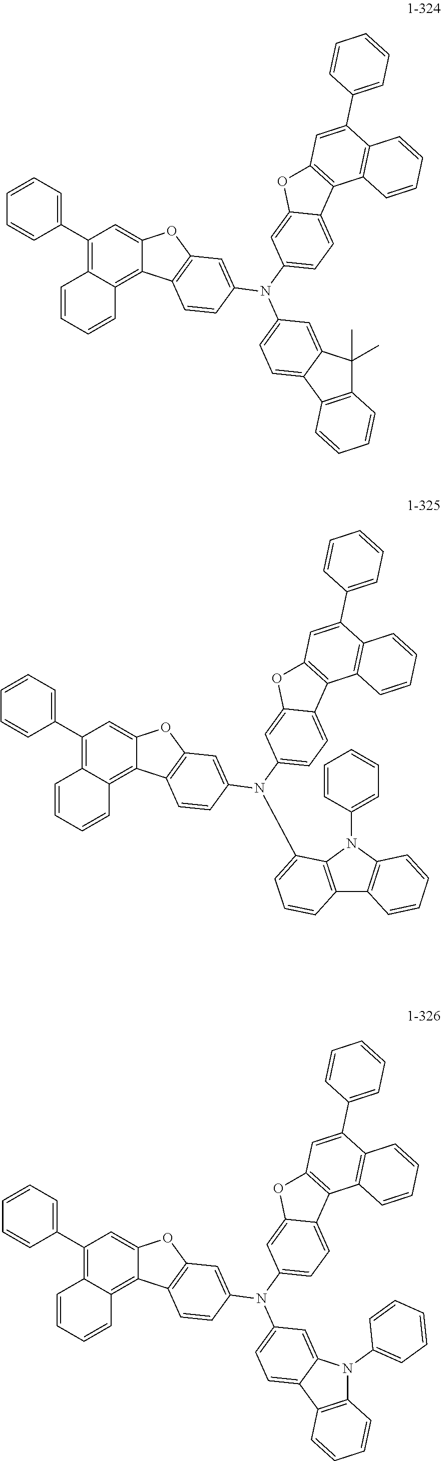 [SMILES: CC1(C)c2ccccc2-c2ccc(N(c3ccc4c(c3)oc3cc(-c5ccccc5)c5ccccc5c34)c3ccc4c(c3)oc3cc(-c5ccccc5)c5ccccc5c34)cc21 ; c1ccc(-c2cc3oc4cc(N(c5ccc6c(c5)oc5cc(-c7ccccc7)c7ccccc7c56)c5cccc6c7ccccc7n(-c7ccccc7)c56)ccc4c3c3ccccc23)cc1 ; c1ccc(-c2cc3oc4cc(N(c5ccc6c(c5)oc5cc(-c7ccccc7)c7ccccc7c56)c5ccc6c7ccccc7n(-c7ccccc7)c6c5)ccc4c3c3ccccc23)cc1]

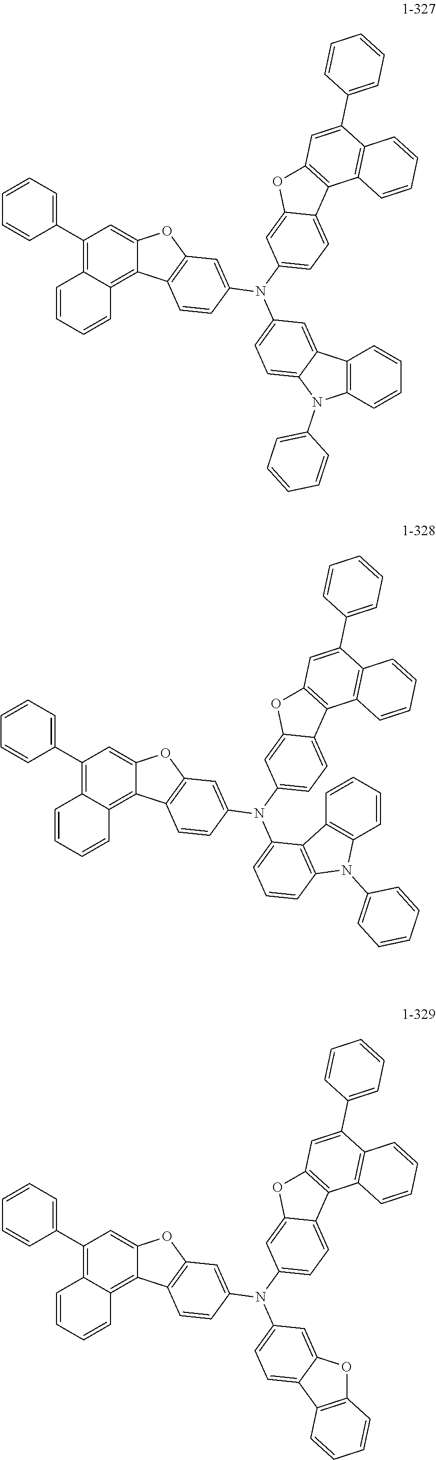 [SMILES: c1ccc(-c2cc3oc4cc(N(c5ccc6c(c5)oc5cc(-c7ccccc7)c7ccccc7c56)c5ccc6c(c5)c5ccccc5n6-c5ccccc5)ccc4c3c3ccccc23)cc1 ; c1ccc(-c2cc3oc4cc(N(c5ccc6c(c5)oc5cc(-c7ccccc7)c7ccccc7c56)c5cccc6c5c5ccccc5n6-c5ccccc5)ccc4c3c3ccccc23)cc1 ; c1ccc(-c2cc3oc4cc(N(c5ccc6c(c5)oc5ccccc56)c5ccc6c(c5)oc5cc(-c7ccccc7)c7ccccc7c56)ccc4c3c3ccccc23)cc1]

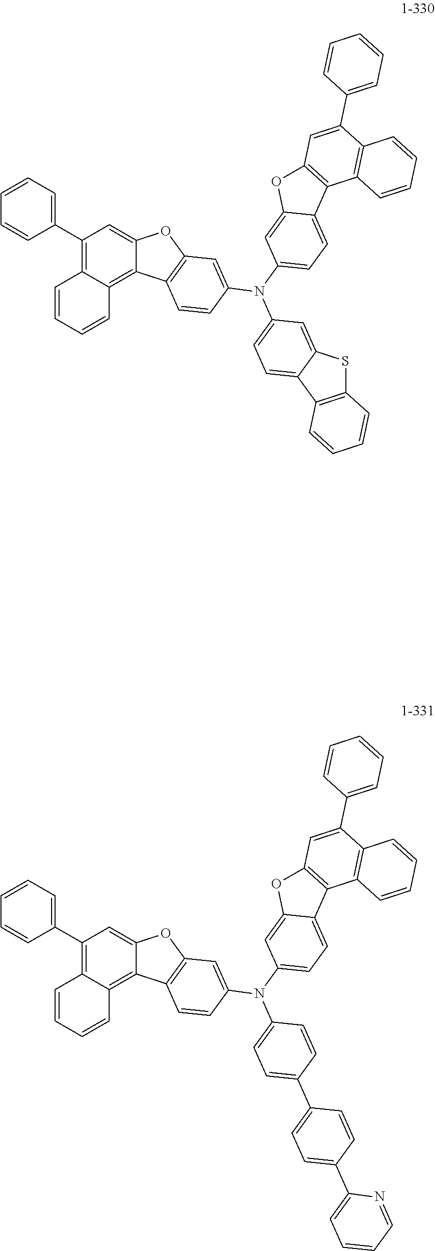 [SMILES: c1ccc(-c2cc3oc4cc(N(c5ccc6c(c5)oc5cc(-c7ccccc7)c7ccccc7c56)c5ccc6c(c5)sc5ccccc56)ccc4c3c3ccccc23)cc1 ; c1ccc(-c2cc3oc4cc(N(c5ccc(-c6ccc(-c7ccccn7)cc6)cc5)c5ccc6c(c5)oc5cc(-c7ccccc7)c7ccccc7c56)ccc4c3c3ccccc23)cc1]

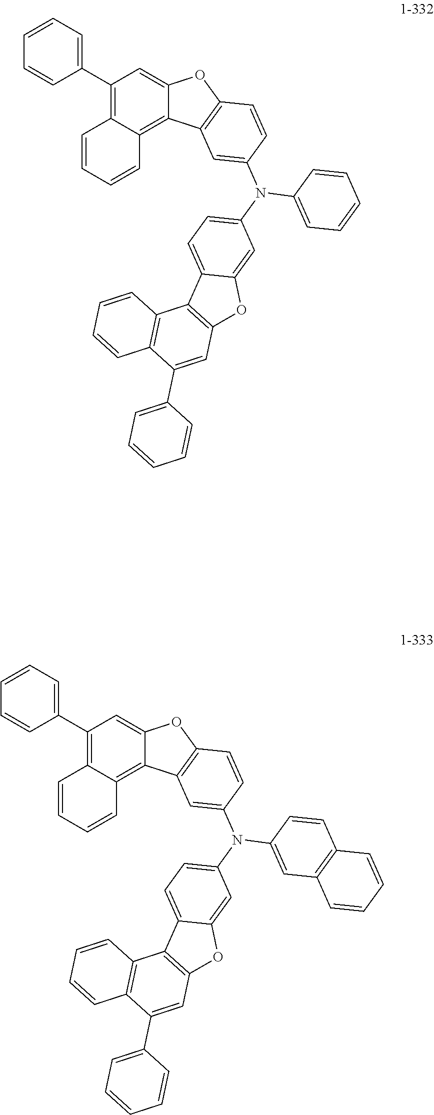 [SMILES: c1ccc(-c2cc3oc4cc(N(c5ccccc5)c5ccc6oc7cc(-c8ccccc8)c8ccccc8c7c6c5)ccc4c3c3ccccc23)cc1 ; c1ccc(-c2cc3oc4cc(N(c5ccc6ccccc6c5)c5ccc6oc7cc(-c8ccccc8)c8ccccc8c7c6c5)ccc4c3c3ccccc23)cc1]

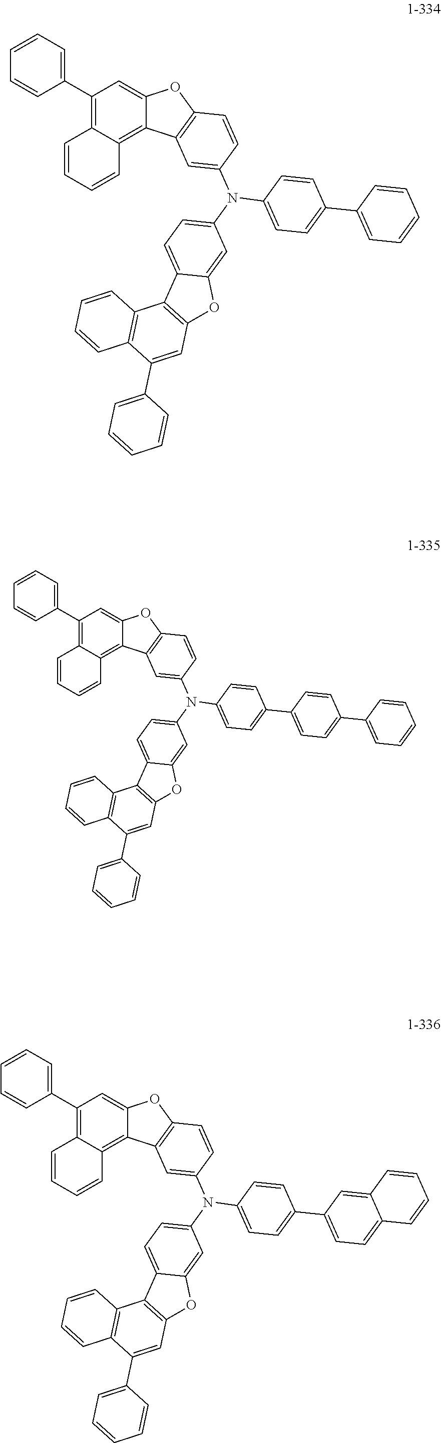 [SMILES: c1ccc(-c2ccc(N(c3ccc4c(c3)oc3cc(-c5ccccc5)c5ccccc5c34)c3ccc4oc5cc(-c6ccccc6)c6ccccc6c5c4c3)cc2)cc1 ; c1ccc(-c2ccc(-c3ccc(N(c4ccc5c(c4)oc4cc(-c6ccccc6)c6ccccc6c45)c4ccc5oc6cc(-c7ccccc7)c7ccccc7c6c5c4)cc3)cc2)cc1 ; c1ccc(-c2cc3oc4cc(N(c5ccc(-c6ccc7ccccc7c6)cc5)c5ccc6oc7cc(-c8ccccc8)c8ccccc8c7c6c5)ccc4c3c3ccccc23)cc1]

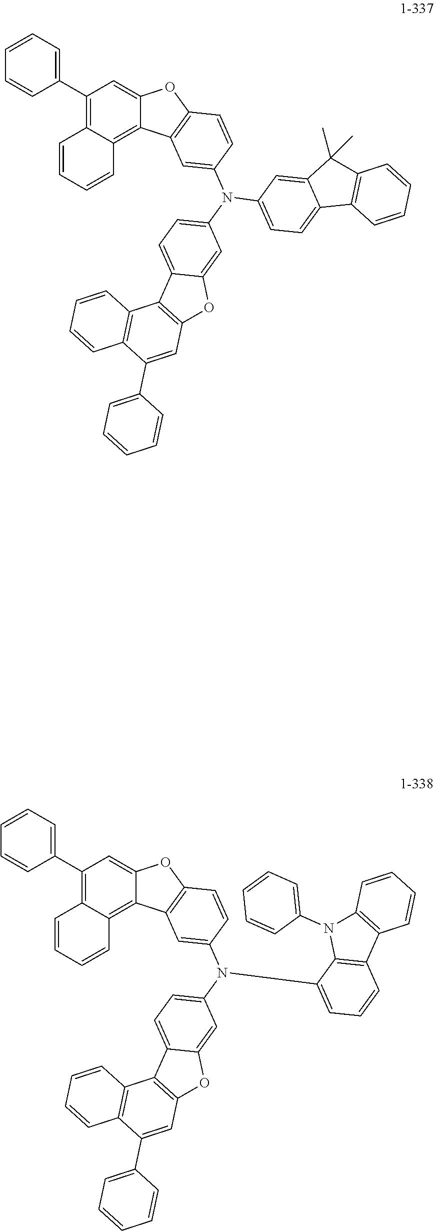 [SMILES: CC1(C)c2ccccc2-c2ccc(N(c3ccc4c(c3)oc3cc(-c5ccccc5)c5ccccc5c34)c3ccc4oc5cc(-c6ccccc6)c6ccccc6c5c4c3)cc21 ; c1ccc(-c2cc3oc4cc(N(c5ccc6oc7cc(-c8ccccc8)c8ccccc8c7c6c5)c5cccc6c7ccccc7n(-c7ccccc7)c56)ccc4c3c3ccccc23)cc1]

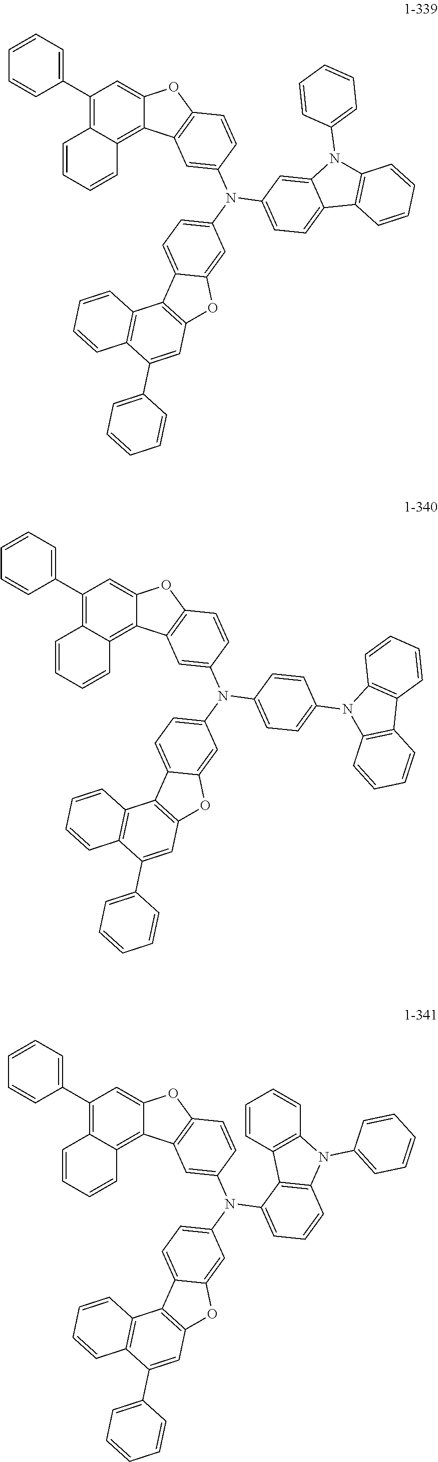 [SMILES: c1ccc(-c2cc3oc4cc(N(c5ccc6oc7cc(-c8ccccc8)c8ccccc8c7c6c5)c5ccc6c7ccccc7n(-c7ccccc7)c6c5)ccc4c3c3ccccc23)cc1 ; c1ccc(-c2cc3oc4cc(N(c5ccc(-n6c7ccccc7c7ccccc76)cc5)c5ccc6oc7cc(-c8ccccc8)c8ccccc8c7c6c5)ccc4c3c3ccccc23)cc1 ; c1ccc(-c2cc3oc4cc(N(c5ccc6oc7cc(-c8ccccc8)c8ccccc8c7c6c5)c5cccc6c5c5ccccc5n6-c5ccccc5)ccc4c3c3ccccc23)cc1]

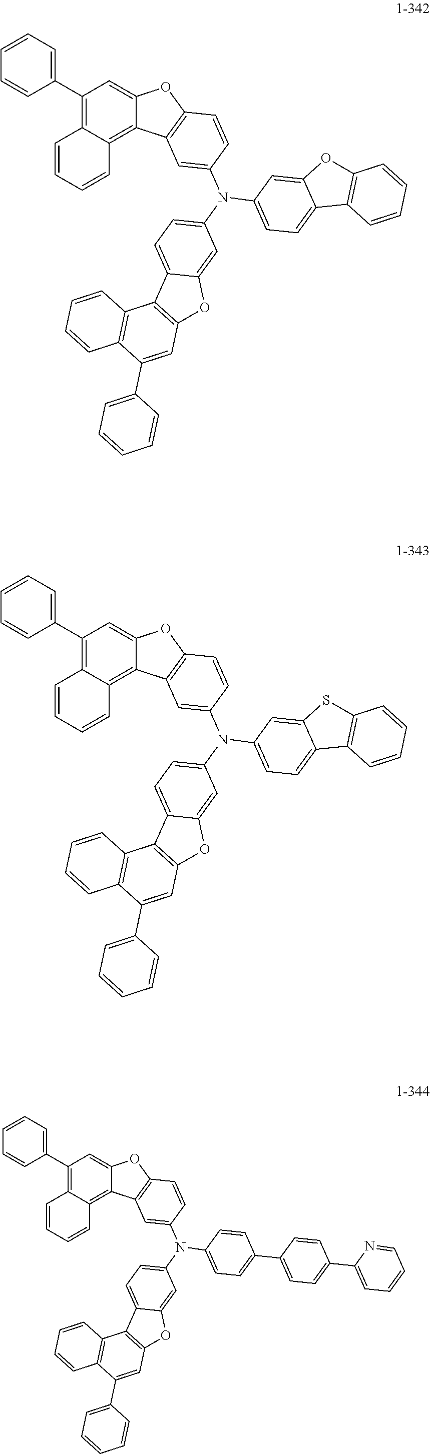 [SMILES: c1ccc(-c2cc3oc4cc(N(c5ccc6c(c5)oc5ccccc56)c5ccc6oc7cc(-c8ccccc8)c8ccccc8c7c6c5)ccc4c3c3ccccc23)cc1 ; c1ccc(-c2cc3oc4cc(N(c5ccc6c(c5)sc5ccccc56)c5ccc6oc7cc(-c8ccccc8)c8ccccc8c7c6c5)ccc4c3c3ccccc23)cc1 ; c1ccc(-c2cc3oc4cc(N(c5ccc(-c6ccc(-c7ccccn7)cc6)cc5)c5ccc6oc7cc(-c8ccccc8)c8ccccc8c7c6c5)ccc4c3c3ccccc23)cc1]

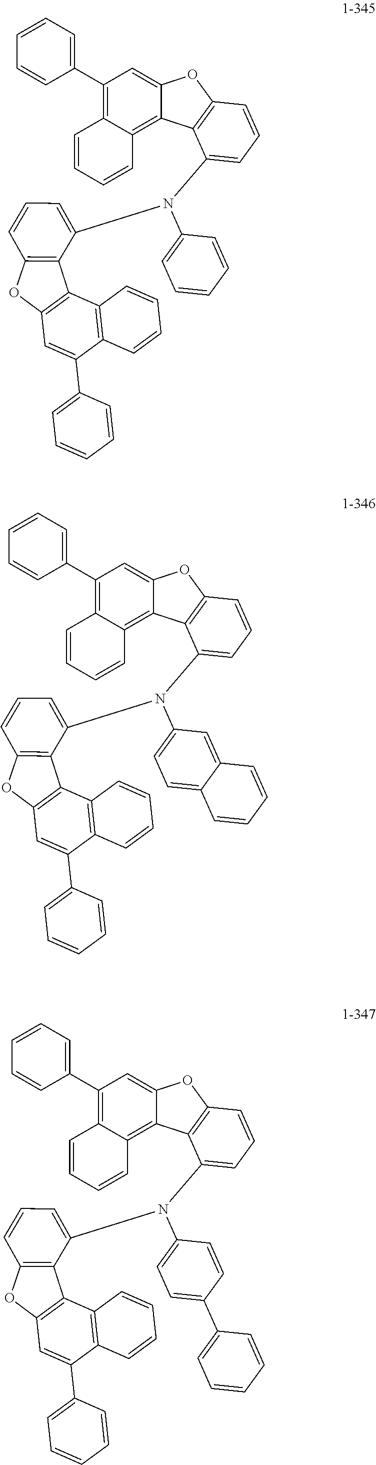 [SMILES: c1ccc(-c2cc3oc4cccc(N(c5ccccc5)c5cccc6oc7cc(-c8ccccc8)c8ccccc8c7c56)c4c3c3ccccc23)cc1 ; c1ccc(-c2cc3oc4cccc(N(c5ccc6ccccc6c5)c5cccc6oc7cc(-c8ccccc8)c8ccccc8c7c56)c4c3c3ccccc23)cc1 ; c1ccc(-c2ccc(N(c3cccc4oc5cc(-c6ccccc6)c6ccccc6c5c34)c3cccc4oc5cc(-c6ccccc6)c6ccccc6c5c34)cc2)cc1]

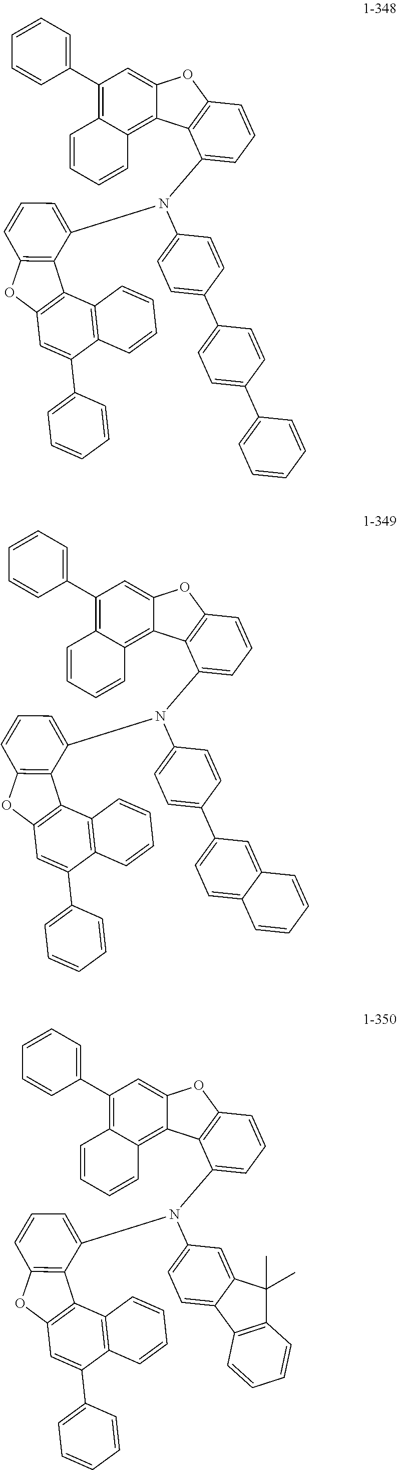 [SMILES: c1ccc(-c2ccc(-c3ccc(N(c4cccc5oc6cc(-c7ccccc7)c7ccccc7c6c45)c4cccc5oc6cc(-c7ccccc7)c7ccccc7c6c45)cc3)cc2)cc1 ; c1ccc(-c2cc3oc4cccc(N(c5ccc(-c6ccc7ccccc7c6)cc5)c5cccc6oc7cc(-c8ccccc8)c8ccccc8c7c56)c4c3c3ccccc23)cc1 ; CC1(C)c2ccccc2-c2ccc(N(c3cccc4oc5cc(-c6ccccc6)c6ccccc6c5c34)c3cccc4oc5cc(-c6ccccc6)c6ccccc6c5c34)cc21]

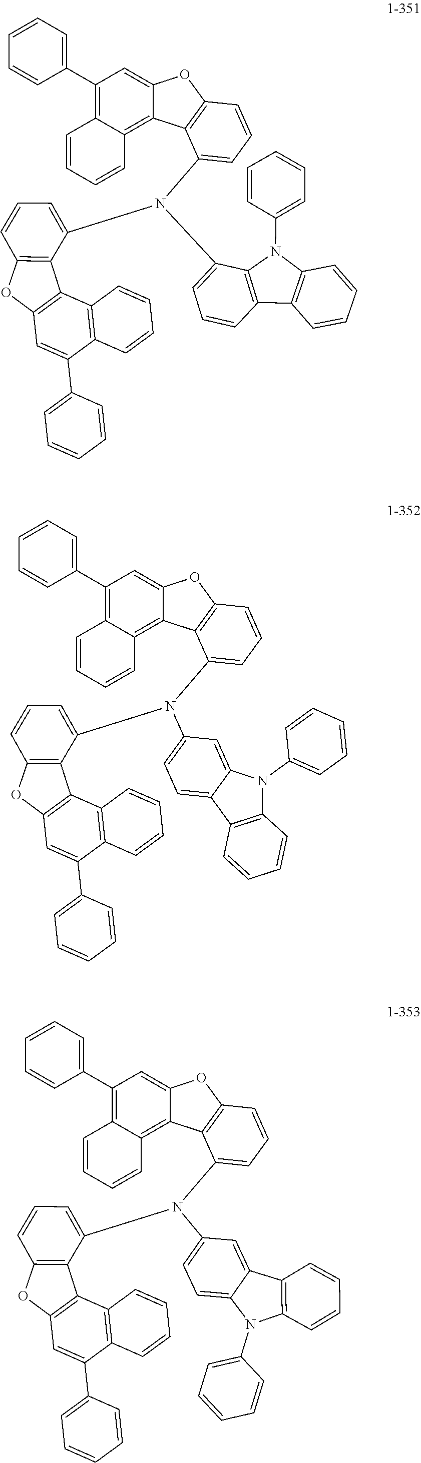

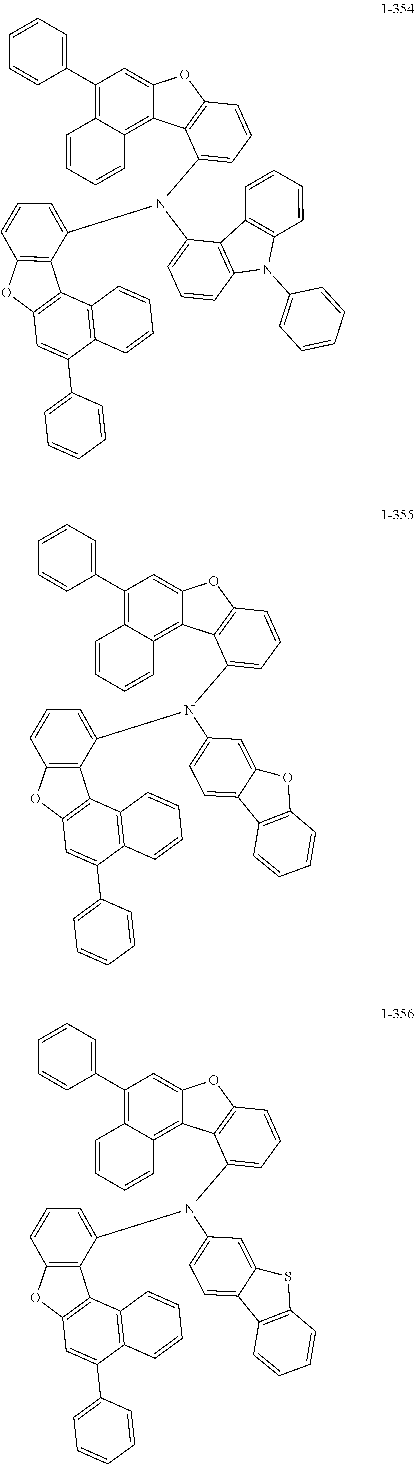 [SMILES: c1ccc(-c2cc3oc4cccc(N(c5cccc6oc7cc(-c8ccccc8)c8ccccc8c7c56)c5cccc6c5c5ccccc5n6-c5ccccc5)c4c3c3ccccc23)cc1 ; c1ccc(-c2cc3oc4cccc(N(c5ccc6c(c5)oc5ccccc56)c5cccc6oc7cc(-c8ccccc8)c8ccccc8c7c56)c4c3c3ccccc23)cc1 ; c1ccc(-c2cc3oc4cccc(N(c5ccc6c(c5)sc5ccccc56)c5cccc6oc7cc(-c8ccccc8)c8ccccc8c7c56)c4c3c3ccccc23)cc1]

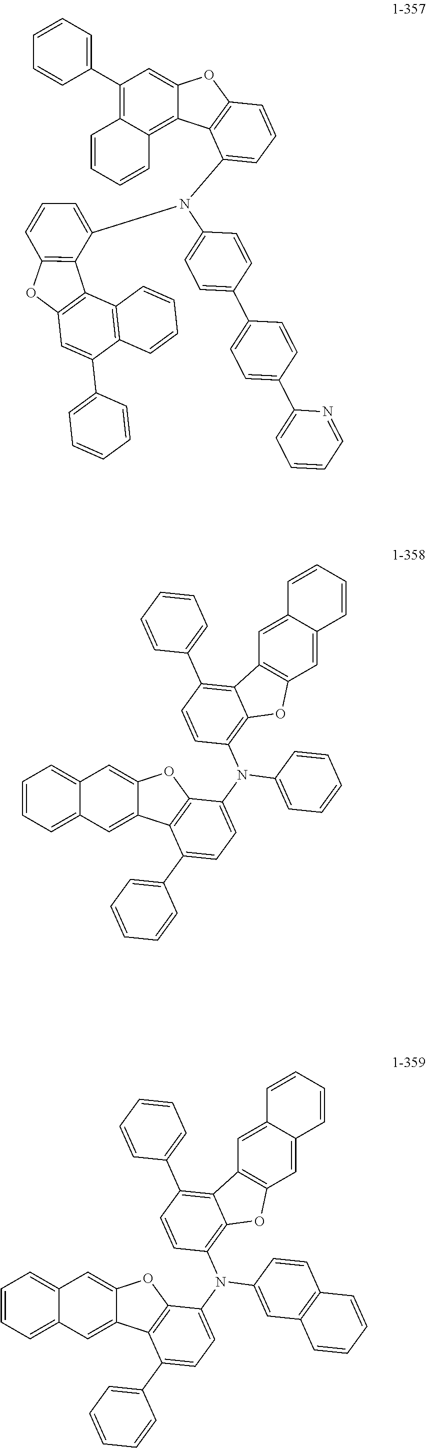 [SMILES: c1ccc(-c2cc3oc4cccc(N(c5ccc(-c6ccc(-c7ccccn7)cc6)cc5)c5cccc6oc7cc(-c8ccccc8)c8ccccc8c7c56)c4c3c3ccccc23)cc1 ; c1ccc(-c2ccc(N(c3ccccc3)c3ccc(-c4ccccc4)c4c3oc3cc5ccccc5cc34)c3oc4cc5ccccc5cc4c23)cc1 ; c1ccc(-c2ccc(N(c3ccc4ccccc4c3)c3ccc(-c4ccccc4)c4c3oc3cc5ccccc5cc34)c3oc4cc5ccccc5cc4c23)cc1]

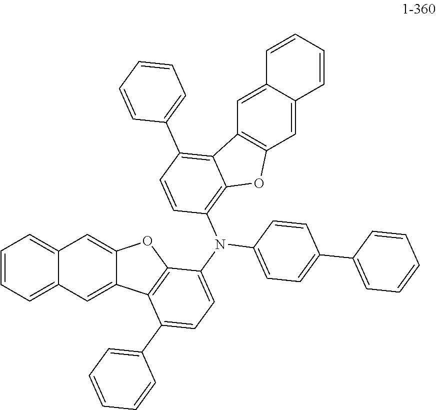 [SMILES: c1ccc(-c2ccc(N(c3ccc(-c4ccccc4)c4c3oc3cc5ccccc5cc34)c3ccc(-c4ccccc4)c4c3oc3cc5ccccc5cc34)cc2)cc1]

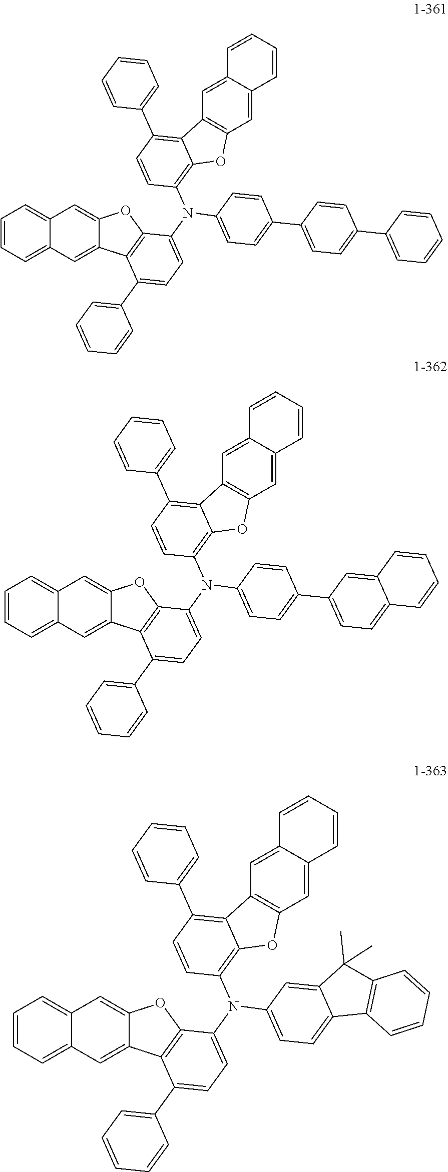 [SMILES: c1ccc(-c2ccc(-c3ccc(N(c4ccc(-c5ccccc5)c5c4oc4cc6ccccc6cc45)c4ccc(-c5ccccc5)c5c4oc4cc6ccccc6cc45)cc3)cc2)cc1 ; c1ccc(-c2ccc(N(c3ccc(-c4ccc5ccccc5c4)cc3)c3ccc(-c4ccccc4)c4c3oc3cc5ccccc5cc34)c3oc4cc5ccccc5cc4c23)cc1 ; CC1(C)c2ccccc2-c2ccc(N(c3ccc(-c4ccccc4)c4c3oc3cc5ccccc5cc34)c3ccc(-c4ccccc4)c4c3oc3cc5ccccc5cc34)cc21]

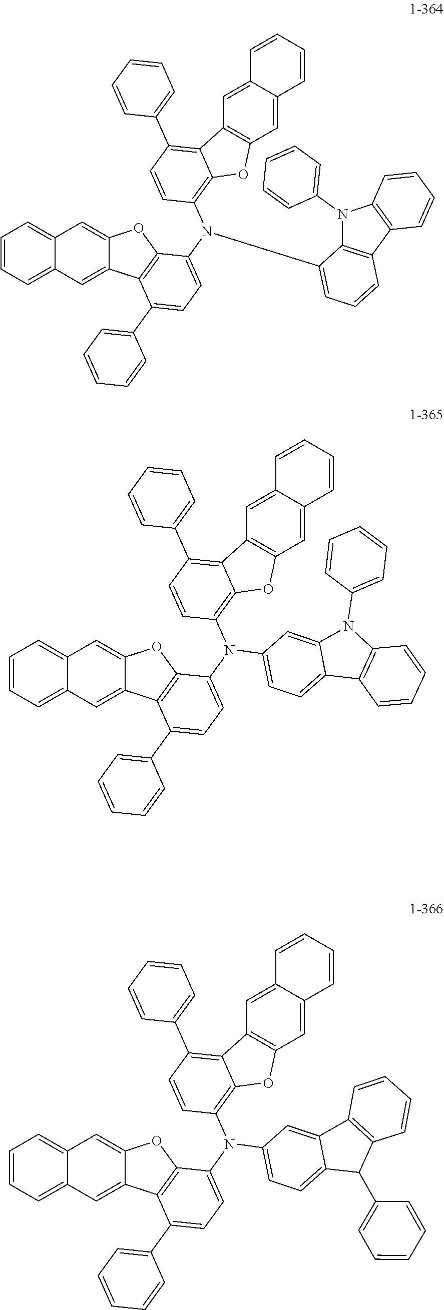 [SMILES: c1ccc(-c2ccc(N(c3ccc(-c4ccccc4)c4c3oc3cc5ccccc5cc34)c3cccc4c5ccccc5n(-c5ccccc5)c34)c3oc4cc5ccccc5cc4c23)cc1 ; c1ccc(-c2ccc(N(c3ccc4c5ccccc5n(-c5ccccc5)c4c3)c3ccc(-c4ccccc4)c4c3oc3cc5ccccc5cc34)c3oc4cc5ccccc5cc4c23)cc1 ; c1ccc(-c2ccc(N(c3ccc4c(c3)-c3ccccc3C4c3ccccc3)c3ccc(-c4ccccc4)c4c3oc3cc5ccccc5cc34)c3oc4cc5ccccc5cc4c23)cc1]

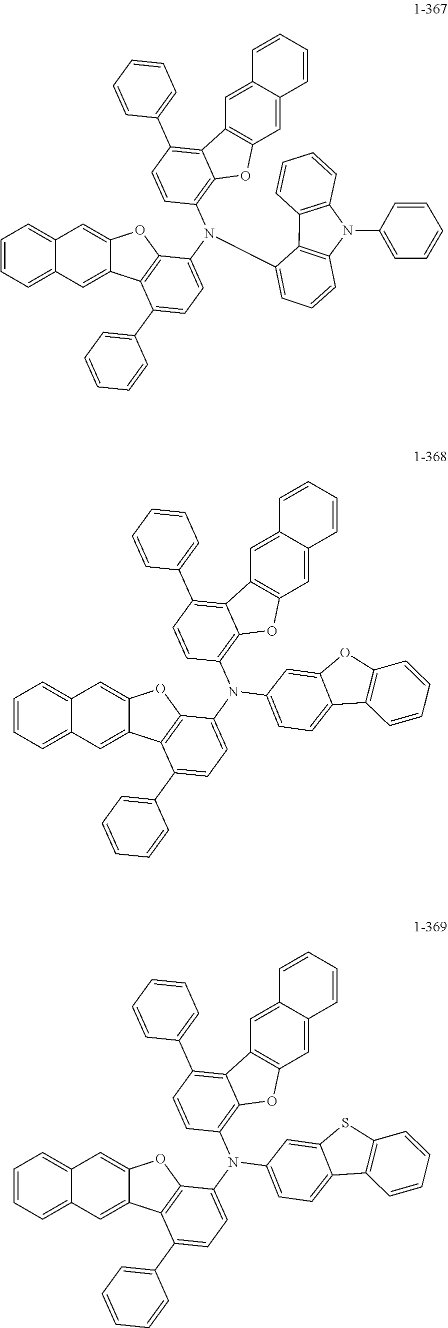 [SMILES: c1ccc(-c2ccc(N(c3ccc(-c4ccccc4)c4c3oc3cc5ccccc5cc34)c3cccc4c3c3ccccc3n4-c3ccccc3)c3oc4cc5ccccc5cc4c23)cc1 ; c1ccc(-c2ccc(N(c3ccc4c(c3)oc3ccccc34)c3ccc(-c4ccccc4)c4c3oc3cc5ccccc5cc34)c3oc4cc5ccccc5cc4c23)cc1 ; c1ccc(-c2ccc(N(c3ccc4c(c3)sc3ccccc34)c3ccc(-c4ccccc4)c4c3oc3cc5ccccc5cc34)c3oc4cc5ccccc5cc4c23)cc1]

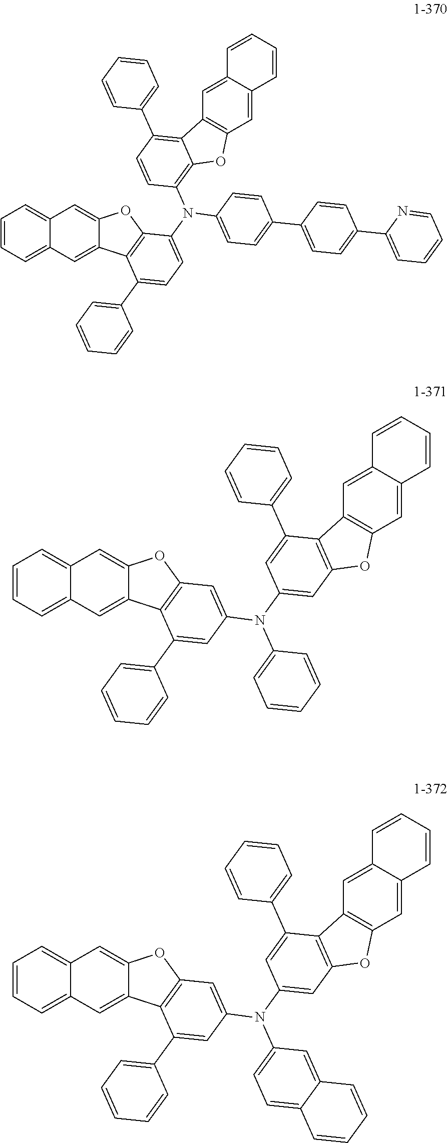 [SMILES: c1ccc(-c2ccc(N(c3ccc(-c4ccc(-c5ccccn5)cc4)cc3)c3ccc(-c4ccccc4)c4c3oc3cc5ccccc5cc34)c3oc4cc5ccccc5cc4c23)cc1 ; c1ccc(-c2cc(N(c3ccccc3)c3cc(-c4ccccc4)c4c(c3)oc3cc5ccccc5cc34)cc3oc4cc5ccccc5cc4c23)cc1 ; c1ccc(-c2cc(N(c3ccc4ccccc4c3)c3cc(-c4ccccc4)c4c(c3)oc3cc5ccccc5cc34)cc3oc4cc5ccccc5cc4c23)cc1]

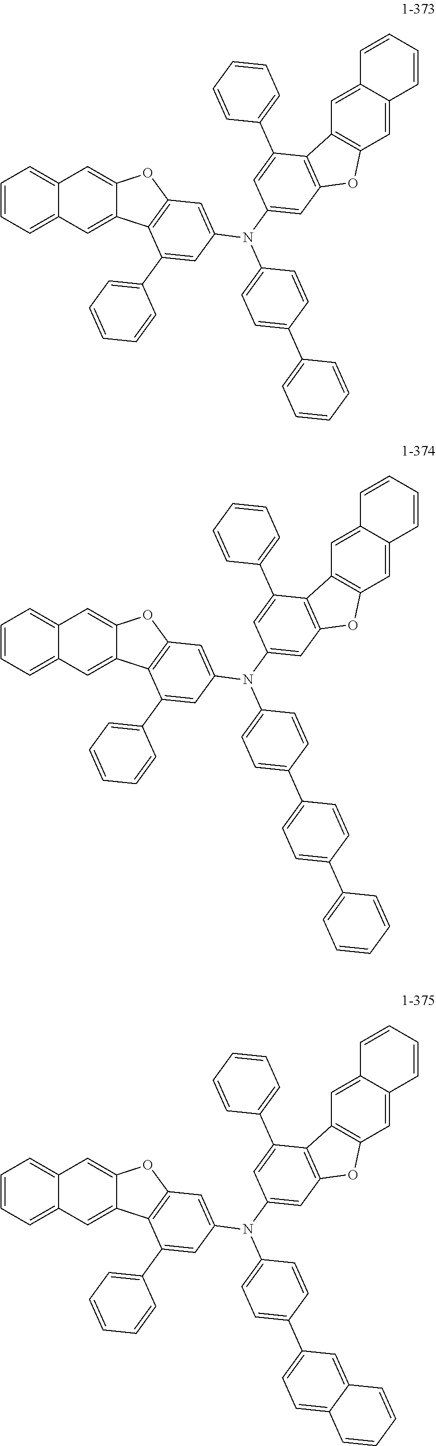 [SMILES: c1ccc(-c2ccc(N(c3cc(-c4ccccc4)c4c(c3)oc3cc5ccccc5cc34)c3cc(-c4ccccc4)c4c(c3)oc3cc5ccccc5cc34)cc2)cc1 ; c1ccc(-c2ccc(-c3ccc(N(c4cc(-c5ccccc5)c5c(c4)oc4cc6ccccc6cc45)c4cc(-c5ccccc5)c5c(c4)oc4cc6ccccc6cc45)cc3)cc2)cc1 ; c1ccc(-c2cc(N(c3ccc(-c4ccc5ccccc5c4)cc3)c3cc(-c4ccccc4)c4c(c3)oc3cc5ccccc5cc34)cc3oc4cc5ccccc5cc4c23)cc1]

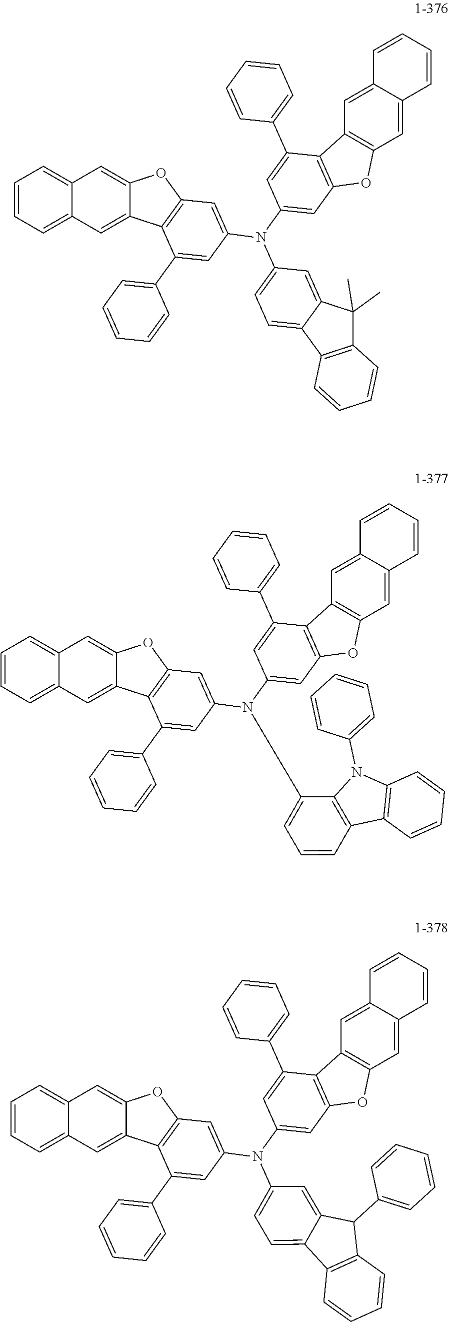 [SMILES: CC1(C)c2ccccc2-c2ccc(N(c3cc(-c4ccccc4)c4c(c3)oc3cc5ccccc5cc34)c3cc(-c4ccccc4)c4c(c3)oc3cc5ccccc5cc34)cc21 ; c1ccc(-c2cc(N(c3cc(-c4ccccc4)c4c(c3)oc3cc5ccccc5cc34)c3cccc4c5ccccc5n(-c5ccccc5)c34)cc3oc4cc5ccccc5cc4c23)cc1 ; c1ccc(-c2cc(N(c3ccc4c(c3)C(c3ccccc3)c3ccccc3-4)c3cc(-c4ccccc4)c4c(c3)oc3cc5ccccc5cc34)cc3oc4cc5ccccc5cc4c23)cc1]

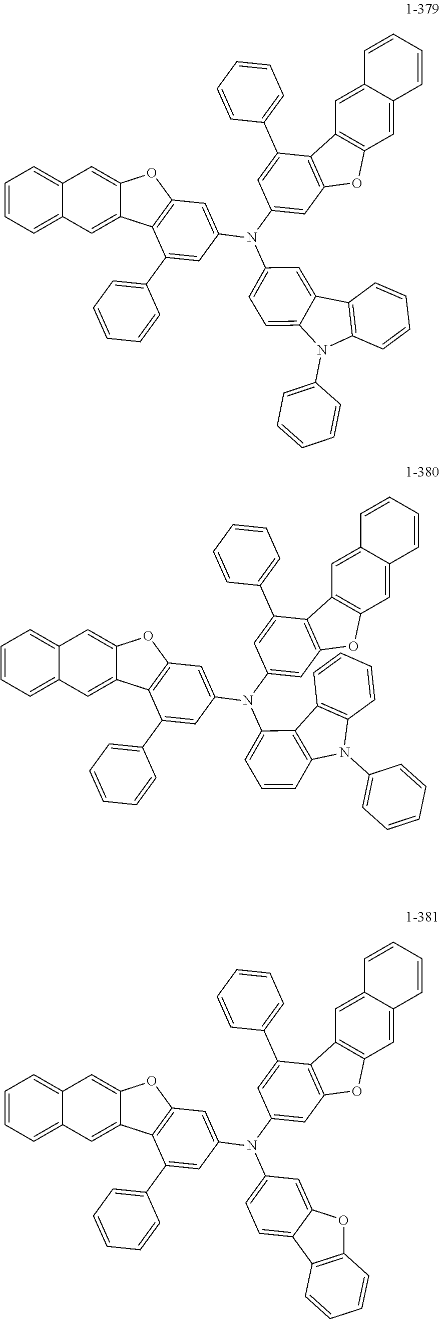 [SMILES: c1ccc(-c2cc(N(c3cc(-c4ccccc4)c4c(c3)oc3cc5ccccc5cc34)c3ccc4c(c3)c3ccccc3n4-c3ccccc3)cc3oc4cc5ccccc5cc4c23)cc1 ; c1ccc(-c2cc(N(c3cc(-c4ccccc4)c4c(c3)oc3cc5ccccc5cc34)c3cccc4c3c3ccccc3n4-c3ccccc3)cc3oc4cc5ccccc5cc4c23)cc1 ; c1ccc(-c2cc(N(c3ccc4c(c3)oc3ccccc34)c3cc(-c4ccccc4)c4c(c3)oc3cc5ccccc5cc34)cc3oc4cc5ccccc5cc4c23)cc1]

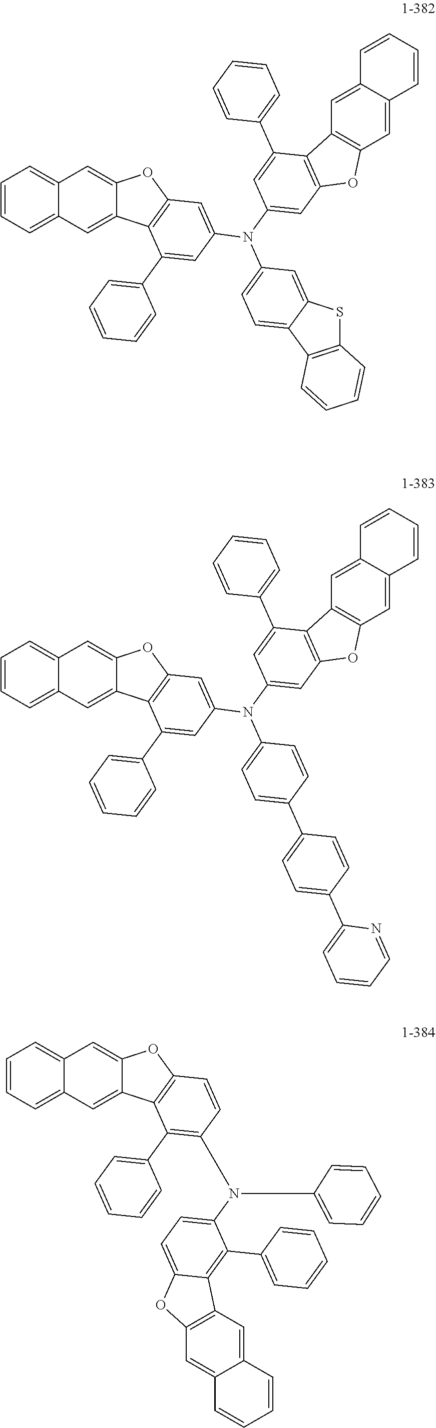 [SMILES: c1ccc(-c2cc(N(c3cc(-c4ccccc4)c4c(c3)oc3cc5ccccc5cc34)c3ccc4c(c3)sc3ccccc34)cc3oc4cc5ccccc5cc4c23)cc1 ; c1ccc(-c2cc(N(c3ccc(-c4ccc(-c5ccccn5)cc4)cc3)c3cc(-c4ccccc4)c4c(c3)oc3cc5ccccc5cc34)cc3oc4cc5ccccc5cc4c23)cc1 ; c1ccc(-c2c(N(c3ccccc3)c3ccc4oc5cc6ccccc6cc5c4c3-c3ccccc3)ccc3oc4cc5ccccc5cc4c23)cc1]

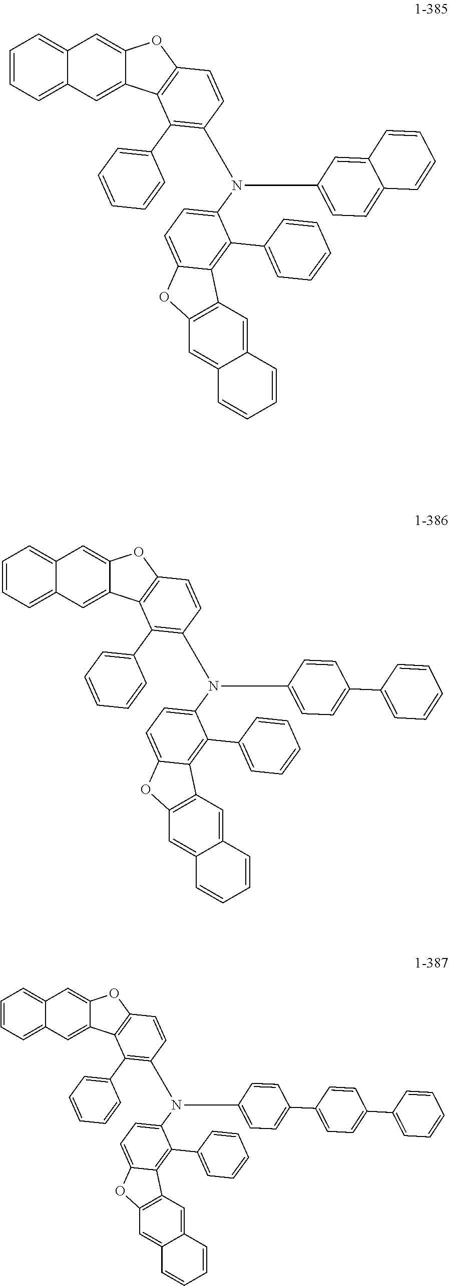 [SMILES: c1ccc(-c2c(N(c3ccc4ccccc4c3)c3ccc4oc5cc6ccccc6cc5c4c3-c3ccccc3)ccc3oc4cc5ccccc5cc4c23)cc1 ; c1ccc(-c2ccc(N(c3ccc4oc5cc6ccccc6cc5c4c3-c3ccccc3)c3ccc4oc5cc6ccccc6cc5c4c3-c3ccccc3)cc2)cc1 ; c1ccc(-c2ccc(-c3ccc(N(c4ccc5oc6cc7ccccc7cc6c5c4-c4ccccc4)c4ccc5oc6cc7ccccc7cc6c5c4-c4ccccc4)cc3)cc2)cc1]

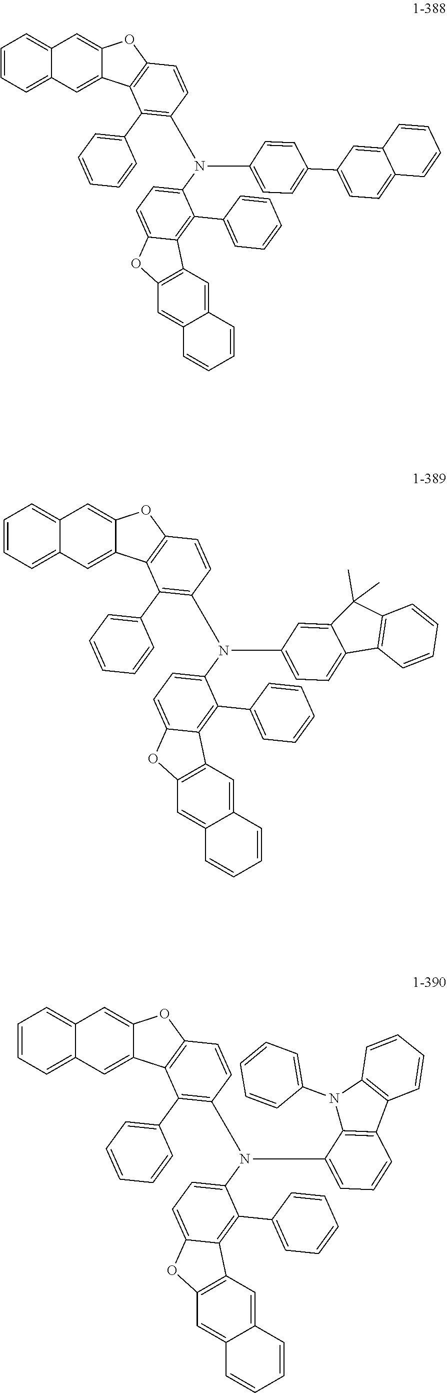 [SMILES: c1ccc(-c2c(N(c3ccc(-c4ccc5ccccc5c4)cc3)c3ccc4oc5cc6ccccc6cc5c4c3-c3ccccc3)ccc3oc4cc5ccccc5cc4c23)cc1 ; CC1(C)c2ccccc2-c2ccc(N(c3ccc4oc5cc6ccccc6cc5c4c3-c3ccccc3)c3ccc4oc5cc6ccccc6cc5c4c3-c3ccccc3)cc21 ; c1ccc(-c2c(N(c3ccc4oc5cc6ccccc6cc5c4c3-c3ccccc3)c3cccc4c5ccccc5n(-c5ccccc5)c34)ccc3oc4cc5ccccc5cc4c23)cc1]

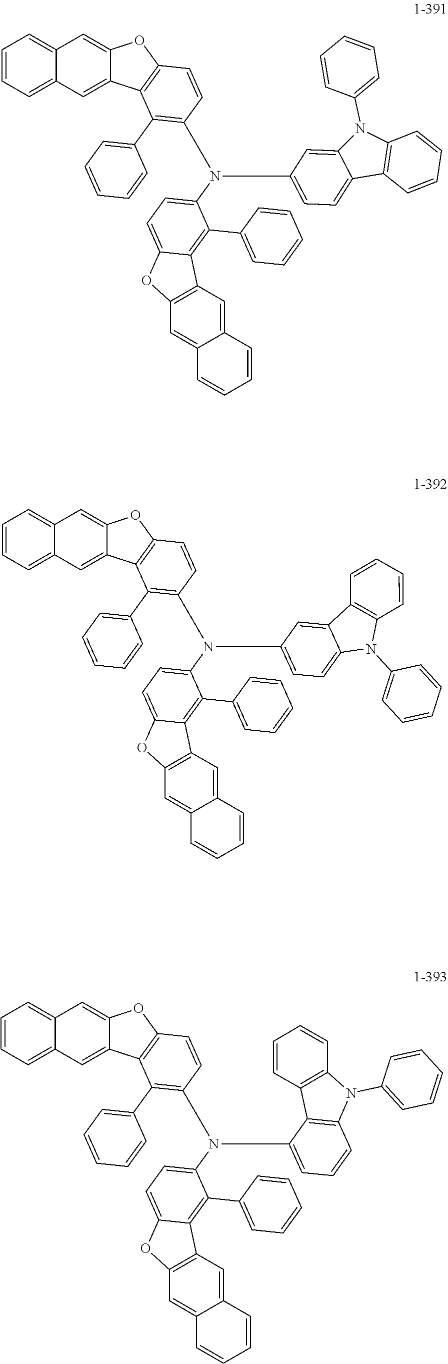 [SMILES: c1ccc(-c2c(N(c3ccc4c5ccccc5n(-c5ccccc5)c4c3)c3ccc4oc5cc6ccccc6cc5c4c3-c3ccccc3)ccc3oc4cc5ccccc5cc4c23)cc1 ; c1ccc(-c2c(N(c3ccc4c(c3)c3ccccc3n4-c3ccccc3)c3ccc4oc5cc6ccccc6cc5c4c3-c3ccccc3)ccc3oc4cc5ccccc5cc4c23)cc1 ; c1ccc(-c2c(N(c3ccc4oc5cc6ccccc6cc5c4c3-c3ccccc3)c3cccc4c3c3ccccc3n4-c3ccccc3)ccc3oc4cc5ccccc5cc4c23)cc1]

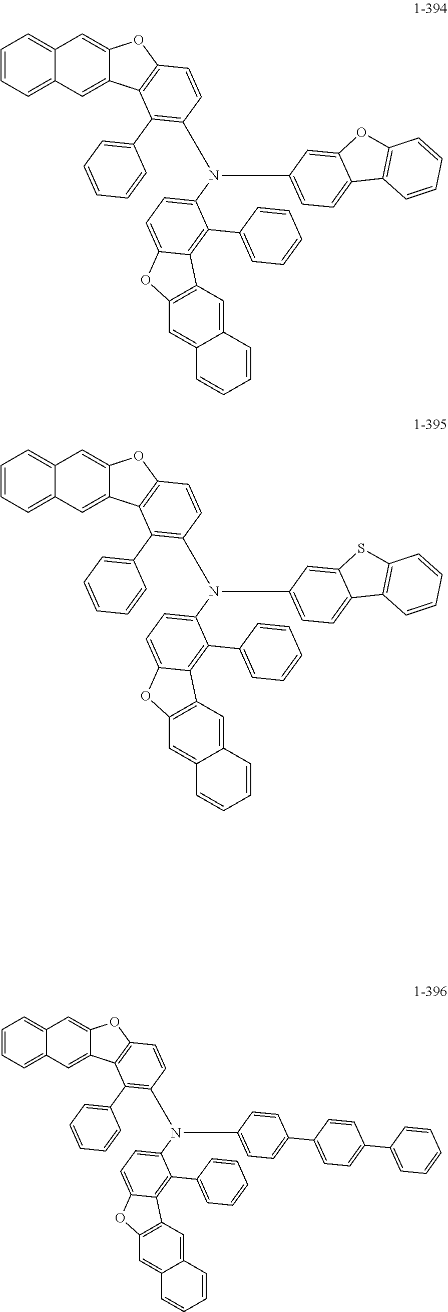 [SMILES: c1ccc(-c2c(N(c3ccc4c(c3)oc3ccccc34)c3ccc4oc5cc6ccccc6cc5c4c3-c3ccccc3)ccc3oc4cc5ccccc5cc4c23)cc1 ; c1ccc(-c2c(N(c3ccc4c(c3)sc3ccccc34)c3ccc4oc5cc6ccccc6cc5c4c3-c3ccccc3)ccc3oc4cc5ccccc5cc4c23)cc1 ; c1ccc(-c2ccc(-c3ccc(N(c4ccc5oc6cc7ccccc7cc6c5c4-c4ccccc4)c4ccc5oc6cc7ccccc7cc6c5c4-c4ccccc4)cc3)cc2)cc1]

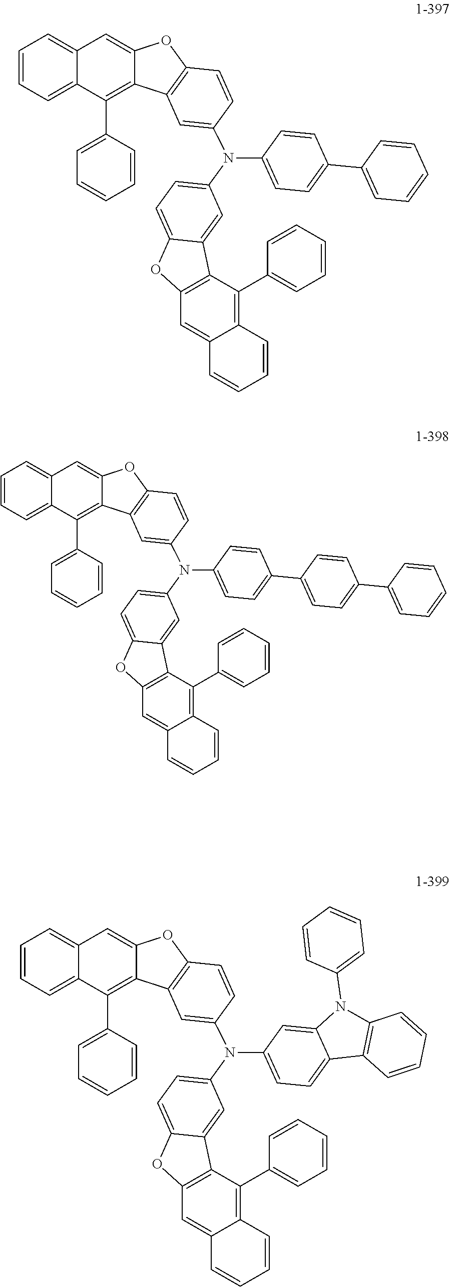 [SMILES: c1ccc(-c2ccc(N(c3ccc4oc5cc6ccccc6c(-c6ccccc6)c5c4c3)c3ccc4oc5cc6ccccc6c(-c6ccccc6)c5c4c3)cc2)cc1 ; c1ccc(-c2ccc(-c3ccc(N(c4ccc5oc6cc7ccccc7c(-c7ccccc7)c6c5c4)c4ccc5oc6cc7ccccc7c(-c7ccccc7)c6c5c4)cc3)cc2)cc1 ; c1ccc(-c2c3ccccc3cc3oc4ccc(N(c5ccc6oc7cc8ccccc8c(-c8ccccc8)c7c6c5)c5ccc6c7ccccc7n(-c7ccccc7)c6c5)cc4c23)cc1]

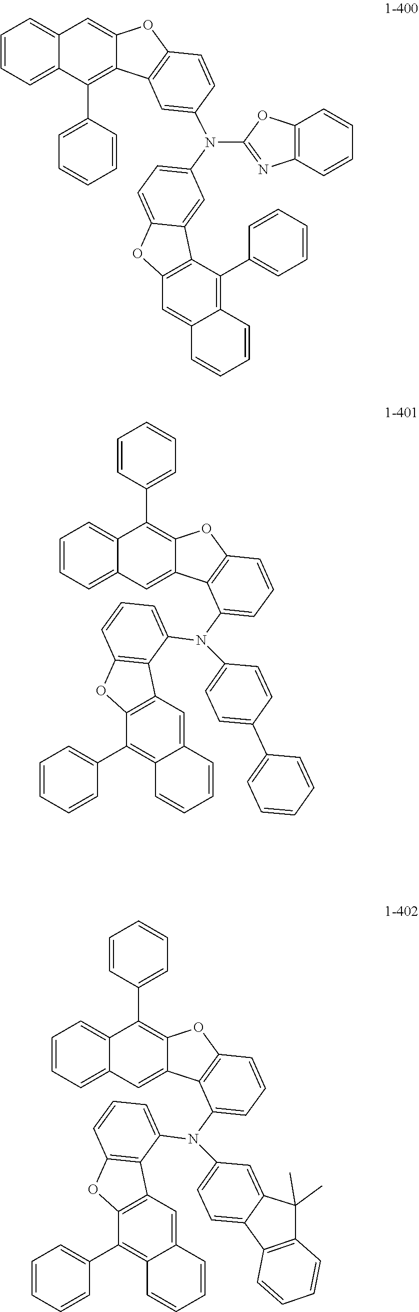 [SMILES: c1ccc(-c2c3ccccc3cc3oc4ccc(N(c5ccc6oc7cc8ccccc8c(-c8ccccc8)c7c6c5)c5nc6ccccc6o5)cc4c23)cc1 ; c1ccc(-c2ccc(N(c3cccc4oc5c(-c6ccccc6)c6ccccc6cc5c34)c3cccc4oc5c(-c6ccccc6)c6ccccc6cc5c34)cc2)cc1 ; CC1(C)c2ccccc2-c2ccc(N(c3cccc4oc5c(-c6ccccc6)c6ccccc6cc5c34)c3cccc4oc5c(-c6ccccc6)c6ccccc6cc5c34)cc21]

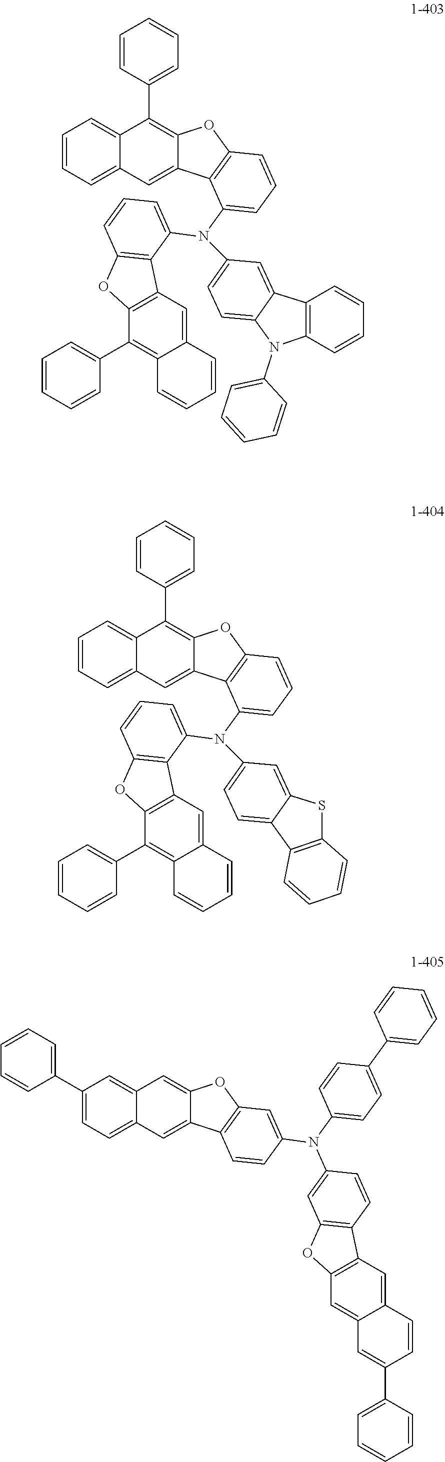 [SMILES: c1ccc(-c2c3ccccc3cc3c2oc2cccc(N(c4ccc5c(c4)c4ccccc4n5-c4ccccc4)c4cccc5oc6c(-c7ccccc7)c7ccccc7cc6c45)c23)cc1 ; c1ccc(-c2c3ccccc3cc3c2oc2cccc(N(c4ccc5c(c4)sc4ccccc45)c4cccc5oc6c(-c7ccccc7)c7ccccc7cc6c45)c23)cc1 ; c1ccc(-c2ccc(N(c3ccc4c(c3)oc3cc5cc(-c6ccccc6)ccc5cc34)c3ccc4c(c3)oc3cc5cc(-c6ccccc6)ccc5cc34)cc2)cc1]

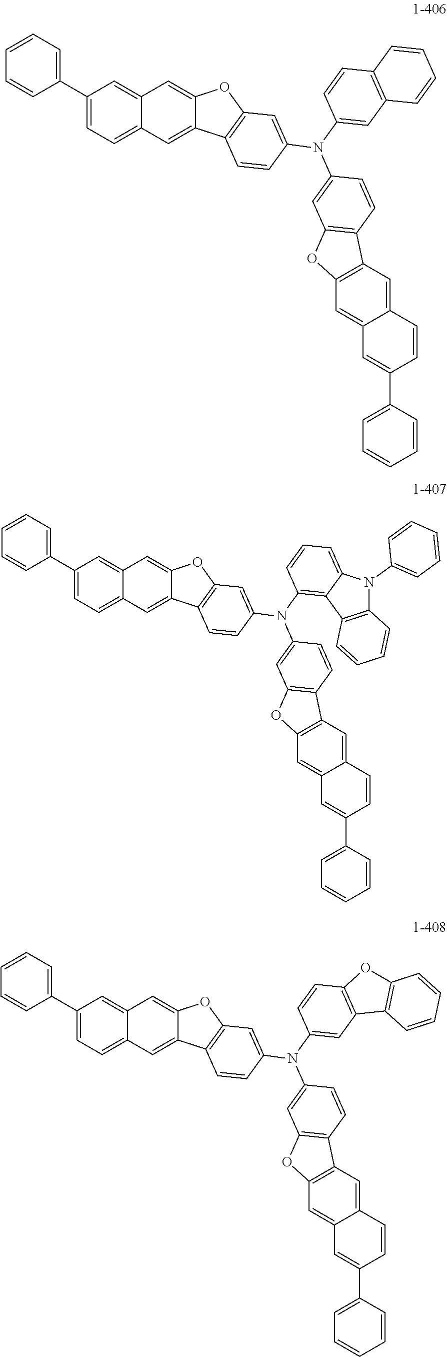 [SMILES: c1ccc(-c2ccc3cc4c(cc3c2)oc2cc(N(c3ccc5ccccc5c3)c3ccc5c(c3)oc3cc6cc(-c7ccccc7)ccc6cc35)ccc24)cc1 ; c1ccc(-c2ccc3cc4c(cc3c2)oc2cc(N(c3ccc5c(c3)oc3cc6cc(-c7ccccc7)ccc6cc35)c3cccc5c3c3ccccc3n5-c3ccccc3)ccc24)cc1 ; c1ccc(-c2ccc3cc4c(cc3c2)oc2cc(N(c3ccc5c(c3)oc3cc6cc(-c7ccccc7)ccc6cc35)c3ccc5oc6ccccc6c5c3)ccc24)cc1]

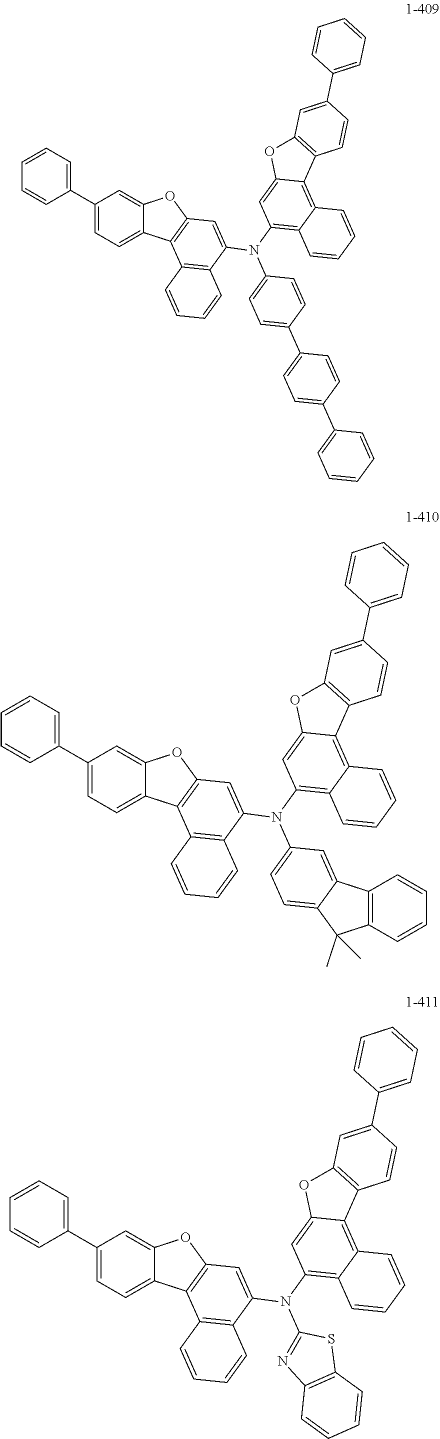 [SMILES: c1ccc(-c2ccc(-c3ccc(N(c4cc5oc6cc(-c7ccccc7)ccc6c5c5ccccc45)c4cc5oc6cc(-c7ccccc7)ccc6c5c5ccccc45)cc3)cc2)cc1 ; CC1(C)c2ccccc2-c2cc(N(c3cc4oc5cc(-c6ccccc6)ccc5c4c4ccccc34)c3cc4oc5cc(-c6ccccc6)ccc5c4c4ccccc34)ccc21 ; c1ccc(-c2ccc3c(c2)oc2cc(N(c4nc5ccccc5s4)c4cc5oc6cc(-c7ccccc7)ccc6c5c5ccccc45)c4ccccc4c23)cc1]

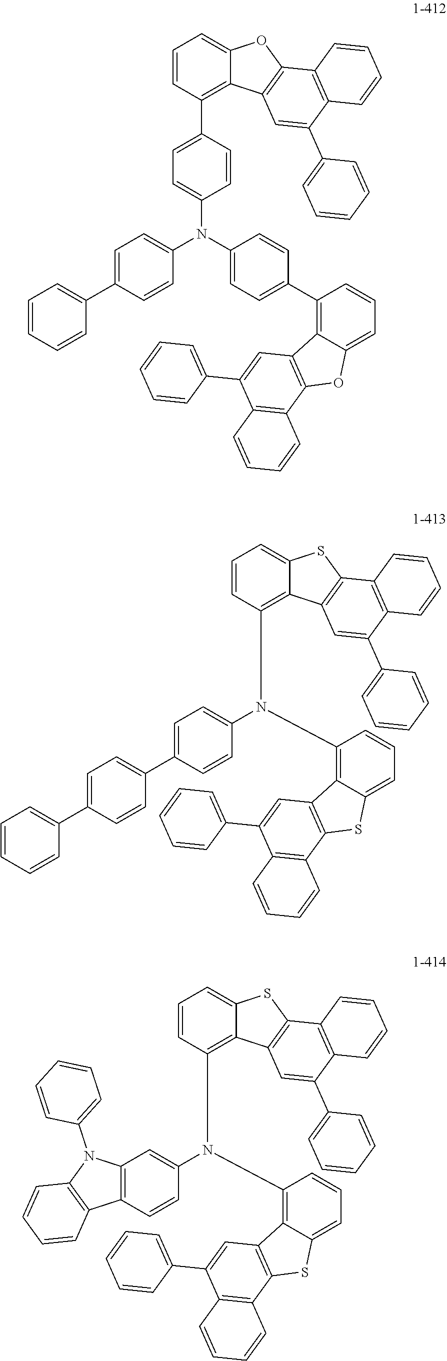 [SMILES: c1ccc(-c2ccc(N(c3ccc(-c4cccc5oc6c7ccccc7c(-c7ccccc7)cc6c45)cc3)c3ccc(-c4cccc5oc6c7ccccc7c(-c7ccccc7)cc6c45)cc3)cc2)cc1 ; c1ccc(-c2ccc(-c3ccc(N(c4cccc5sc6c7ccccc7c(-c7ccccc7)cc6c45)c4cccc5sc6c7ccccc7c(-c7ccccc7)cc6c45)cc3)cc2)cc1 ; c1ccc(-c2cc3c(sc4cccc(N(c5ccc6c7ccccc7n(-c7ccccc7)c6c5)c5cccc6sc7c8ccccc8c(-c8ccccc8)cc7c56)c43)c3ccccc23)cc1]

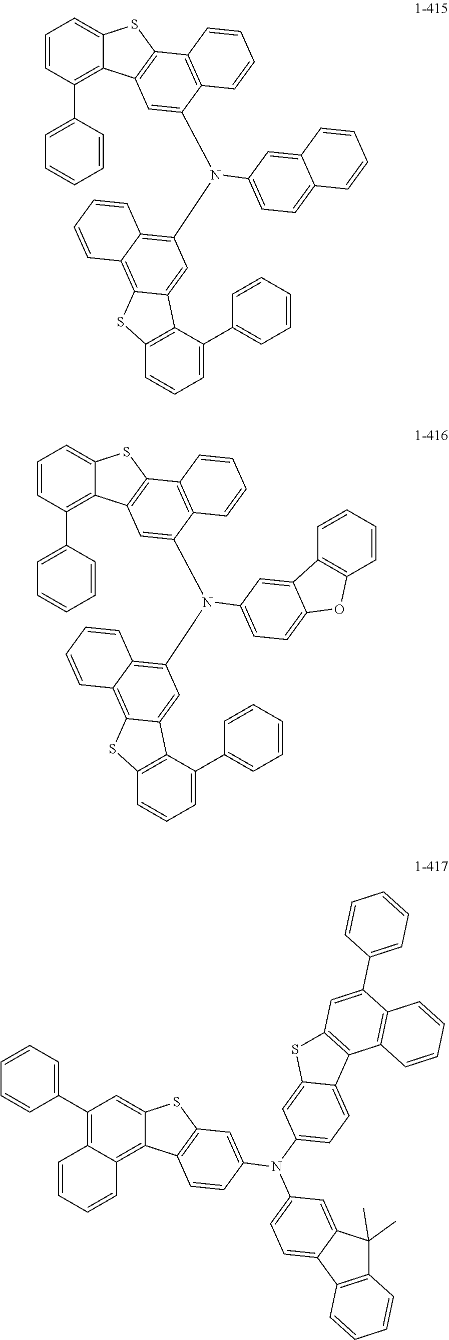 [SMILES: c1ccc(-c2cccc3sc4c5ccccc5c(N(c5ccc6ccccc6c5)c5cc6c(sc7cccc(-c8ccccc8)c76)c6ccccc56)cc4c23)cc1 ; c1ccc(-c2cccc3sc4c5ccccc5c(N(c5ccc6oc7ccccc7c6c5)c5cc6c(sc7cccc(-c8ccccc8)c76)c6ccccc56)cc4c23)cc1 ; CC1(C)c2ccccc2-c2ccc(N(c3ccc4c(c3)sc3cc(-c5ccccc5)c5ccccc5c34)c3ccc4c(c3)sc3cc(-c5ccccc5)c5ccccc5c34)cc21]

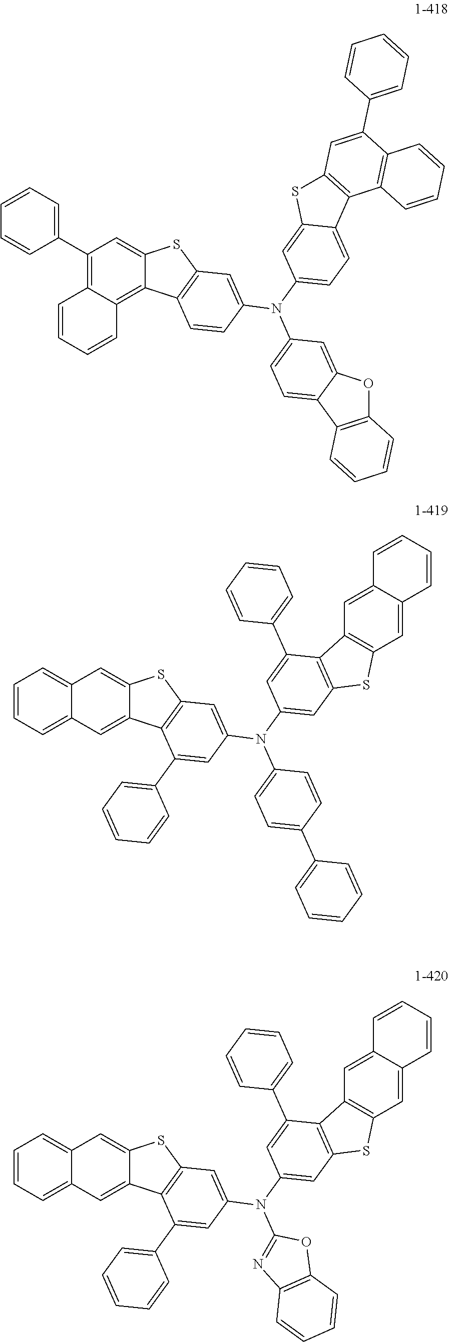 [SMILES: c1ccc(-c2cc3sc4cc(N(c5ccc6c(c5)oc5ccccc56)c5ccc6c(c5)sc5cc(-c7ccccc7)c7ccccc7c56)ccc4c3c3ccccc23)cc1 ; c1ccc(-c2ccc(N(c3cc(-c4ccccc4)c4c(c3)sc3cc5ccccc5cc34)c3cc(-c4ccccc4)c4c(c3)sc3cc5ccccc5cc34)cc2)cc1 ; c1ccc(-c2cc(N(c3cc(-c4ccccc4)c4c(c3)sc3cc5ccccc5cc34)c3nc4ccccc4o3)cc3sc4cc5ccccc5cc4c23)cc1]

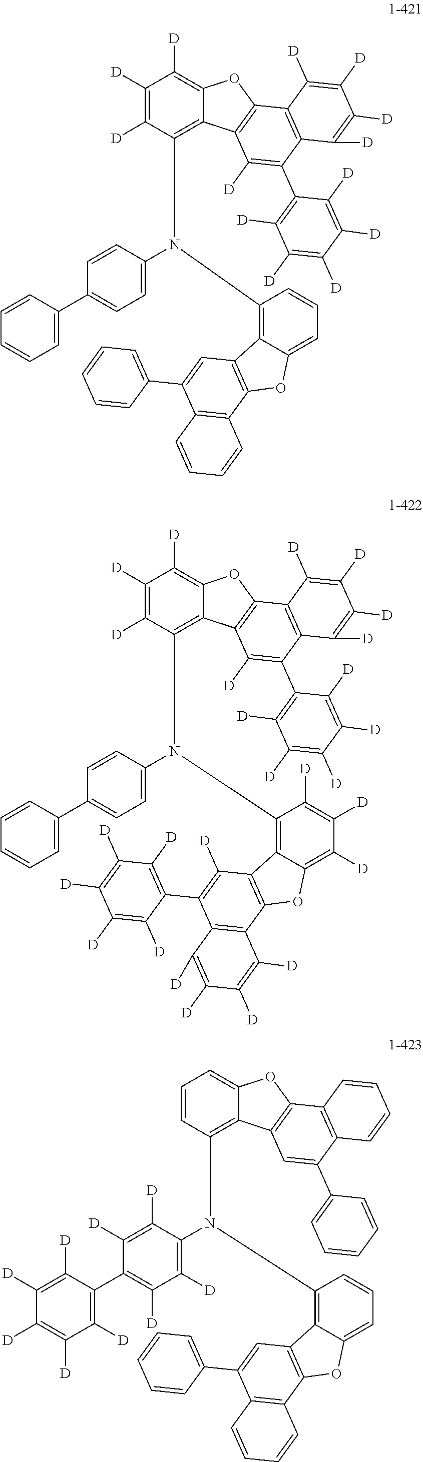

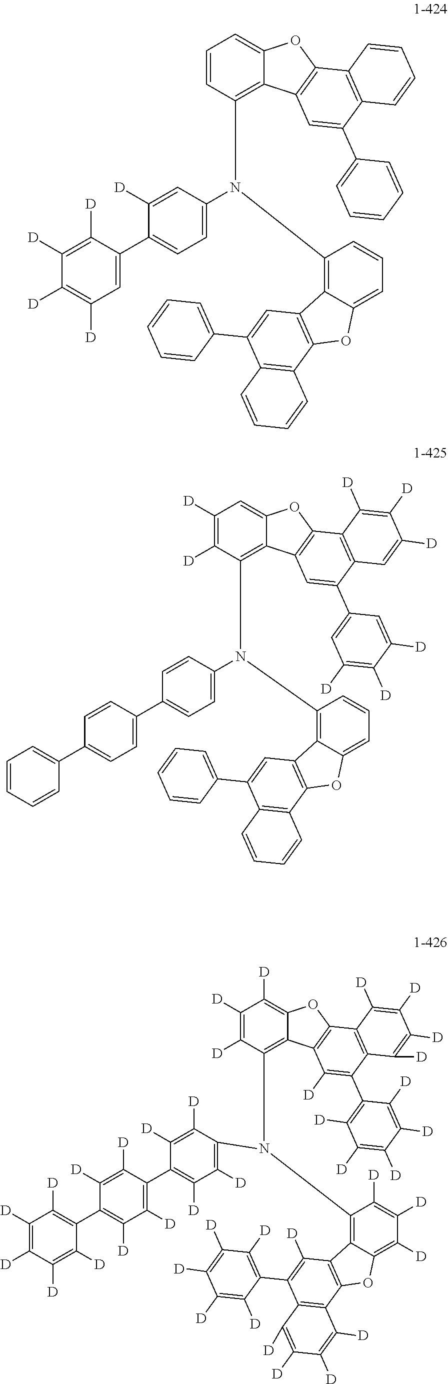

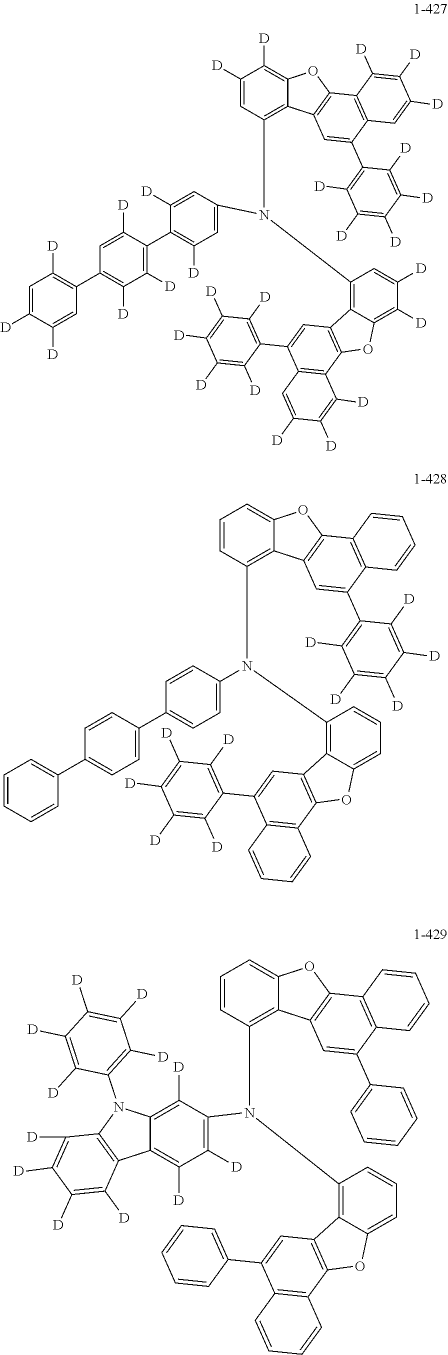

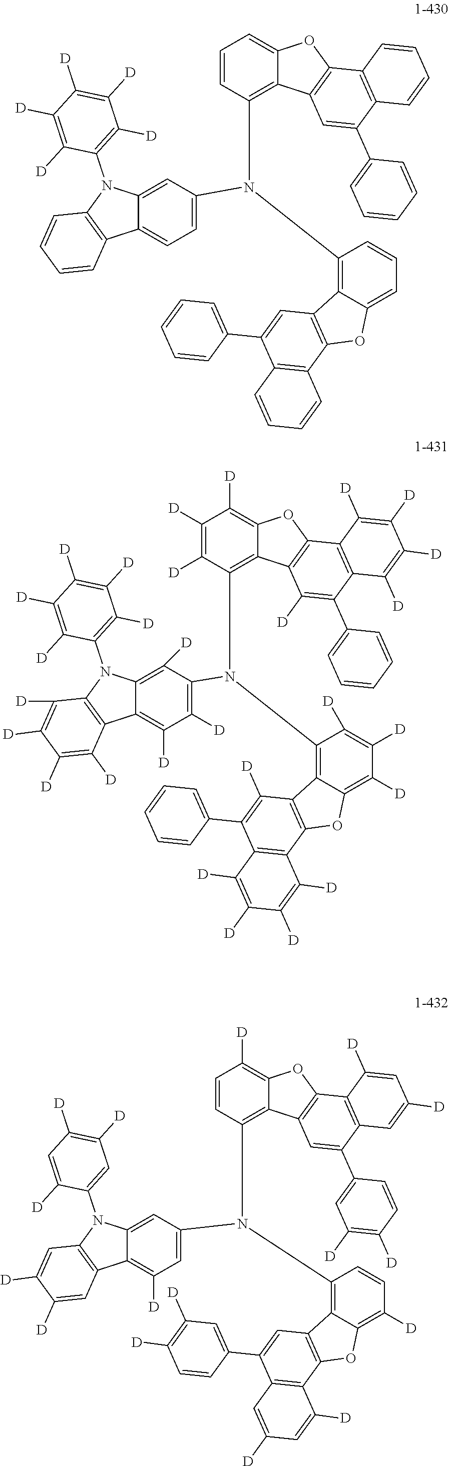

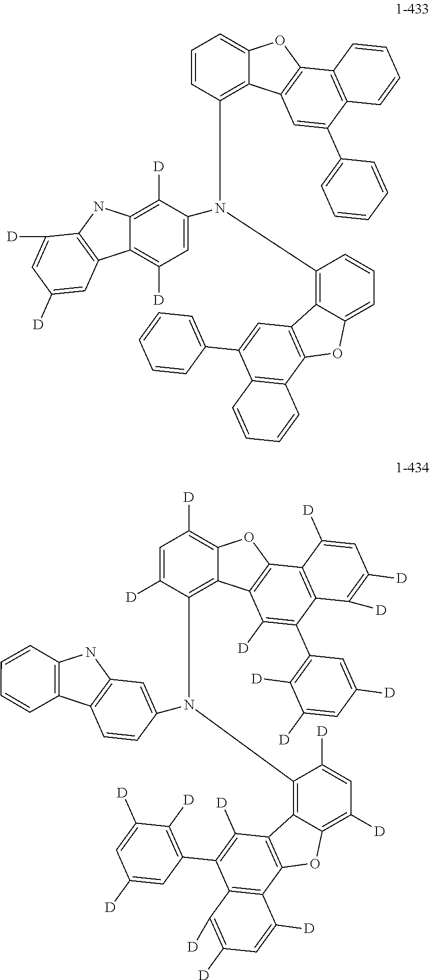 [SMILES: [2H]c1cc([2H])c2[nH]c3c([2H])c(N(c4cccc5oc6c7ccccc7c(-c7ccccc7)cc6c45)c4cccc5oc6c7ccccc7c(-c7ccccc7)cc6c45)cc([2H])c3c2c1 ; [2H]c1cc([2H])c([2H])c(-c2c([2H])c3c(oc4c([2H])cc([2H])c(N(c5ccc6c(c5)[nH]c5ccccc56)c5c([2H])cc([2H])c6oc7c8c([2H])cc([2H])c([2H])c8c(-c8cc([2H])cc([2H])c8[2H])c([2H])c7c56)c43)c3c([2H])cc([2H])c([2H])c23)c1]

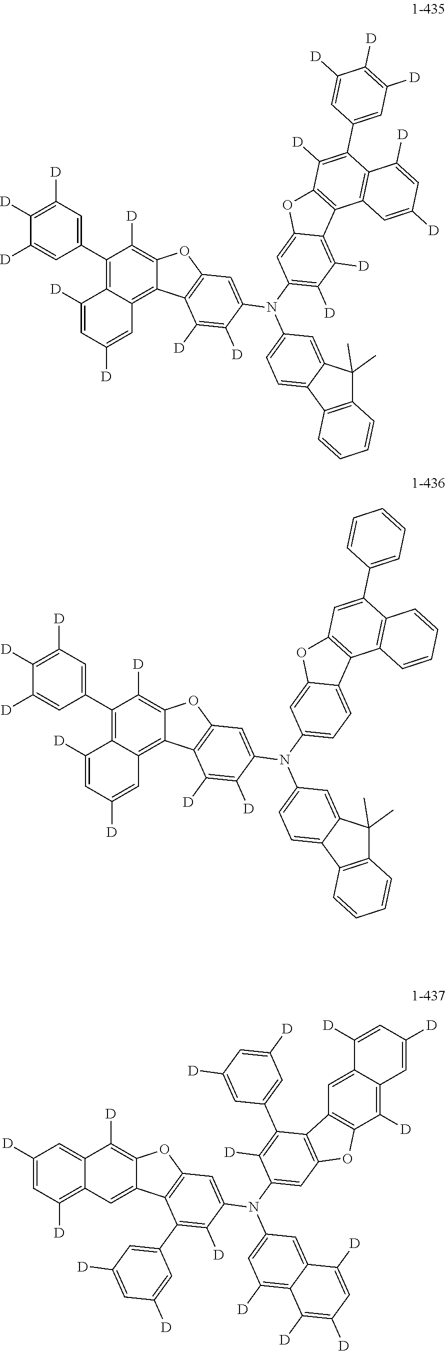

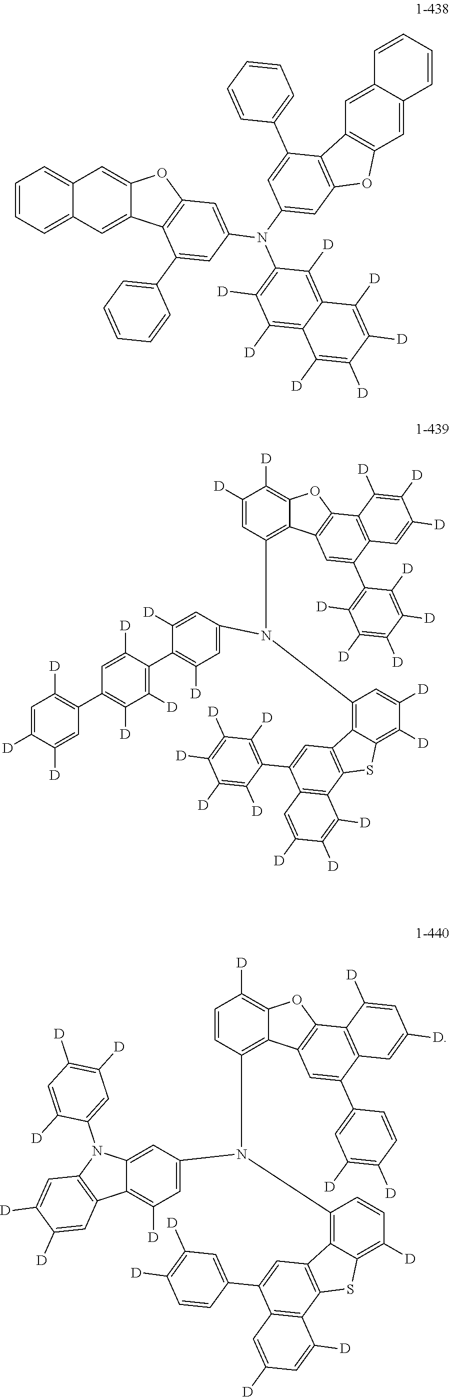

9. Alight-emitting device comprising:
a first electrode;
a second electrode disposed on the first electrode; and
one or more organic layers interposed between the first electrode and the second electrode, the one or more of the organic layers comprising the heterocyclic compound of claim 1.

10. The light-emitting device according to claim 9, wherein the one or more organic layers comprise alight-emitting layer, the light-emitting layer comprises a dopant material and a host material, and the host material comprises the heterocyclic compound.

11. The light-emitting device according to claim 10, wherein the one or more organic layers further comprise:
a hole transport layer disposed between the first electrode and the light-emitting layer, and
an electron transport layer disposed between the light-emitting layer and the second electrode.

12. The light-emitting device according to claim 10, wherein the host material further comprises a compound represented by Formula 3: 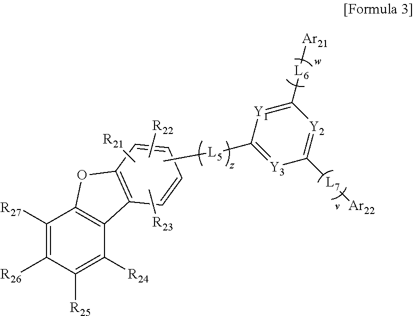
wherein, in Formula 3, any one of R₂₁ to R₂₇ is a substituted or unsubstituted aryl group having 6 to 60 carbon atoms, or a substituted or unsubstituted heteroaryl group having 2 to 60 carbon atoms, and
the others of R₂₁ to R₂₇ are each independently selected from the group consisting of hydrogen, deuterium, a halogen, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, —SiRR′R″, and —P(═O)RR′, wherein adjacent groups are linked to form a substituted or unsubstituted 5- to 9-membered saturated or unsaturated ring,
R, R′ and R″ are each independently hydrogen, deuterium, —CN, a substituted or unsubstituted alkyl group having 1 to 60 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms,
L₅, L₆ and L₇ are each independently a direct linkage, a substituted or unsubstituted arylene group having 6 to 60 carbon atoms, or a substituted or unsubstituted heteroarylene group having 2 to 60 carbon atoms,
w, v and z are each independently 0 or an integer of 1 to 5,
when w, v and z are integers of 2 to 5, a plurality of L₅ groups, L₆ groups, and L₇ groups are each the same or different,
Y₁ to Y₃ are each independently CH, nitrogen (N), oxygen (O), or sulfur (S), and at least two of Y₁ to Y₃ are nitrogen, and
Ar₂₁ and Ar₂₂ are each independently a substituted or unsubstituted aryl group having 6 to 60 carbon atoms, or a substituted or unsubstituted heteroaryl group having 2 to 60 carbon atoms.

13. The light-emitting device according to claim 12, wherein the compound represented by Formula 3 has a deuterium content of 0, or greater than 0 and not more than 100%.

14. The light-emitting device according to claim 12, wherein the compound represented by Formula 3 is represented by at least one selected from the formulae below:

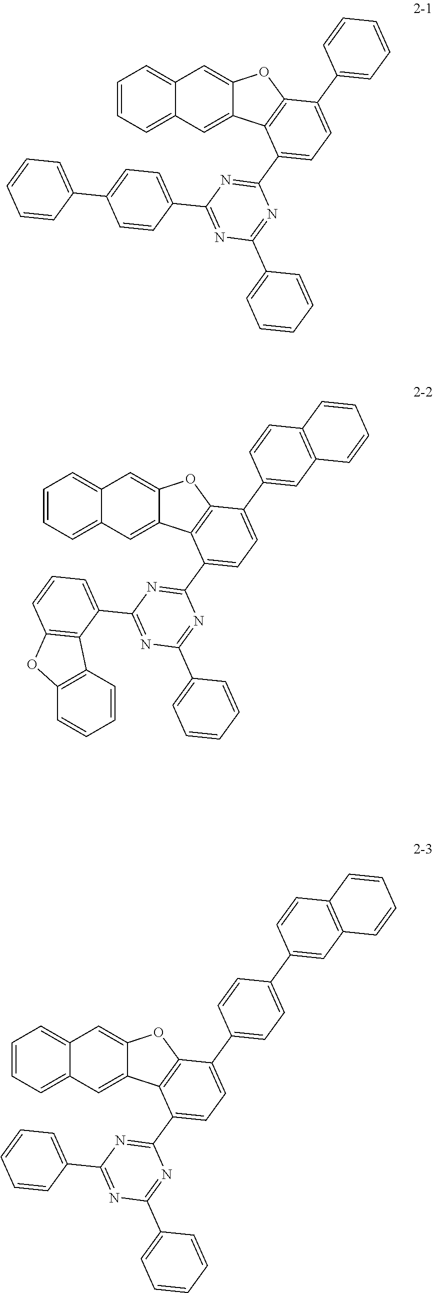 [SMILES: c1ccc(-c2ccc(-c3nc(-c4ccccc4)nc(-c4ccc(-c5ccccc5)c5oc6cc7ccccc7cc6c45)n3)cc2)cc1 ; c1ccc(-c2nc(-c3cccc4oc5ccccc5c34)nc(-c3ccc(-c4ccc5ccccc5c4)c4oc5cc6ccccc6cc5c34)n2)cc1 ; c1ccc(-c2nc(-c3ccccc3)nc(-c3ccc(-c4ccc(-c5ccc6ccccc6c5)cc4)c4oc5cc6ccccc6cc5c34)n2)cc1]

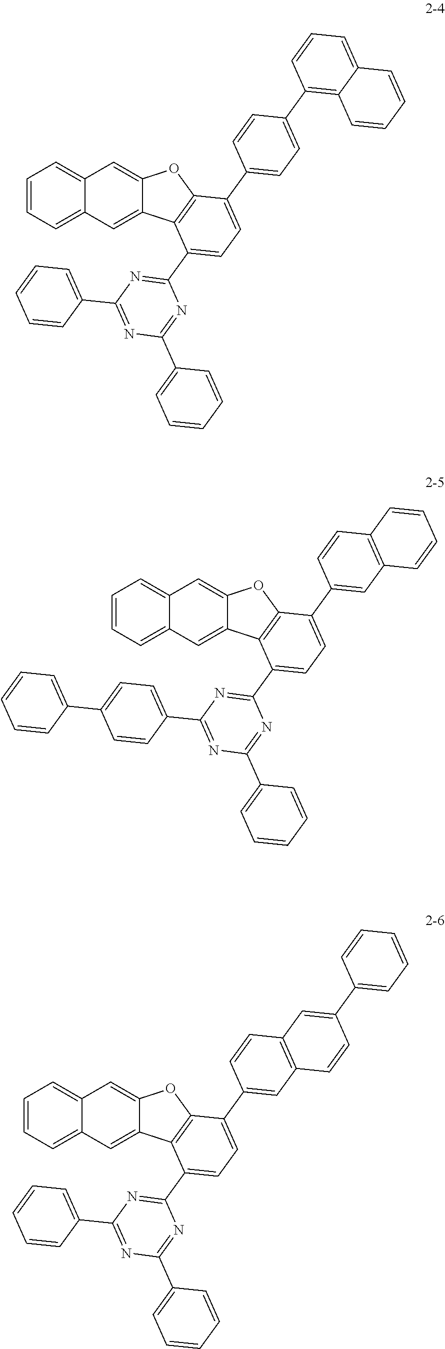 [SMILES: c1ccc(-c2nc(-c3ccccc3)nc(-c3ccc(-c4ccc(-c5cccc6ccccc56)cc4)c4oc5cc6ccccc6cc5c34)n2)cc1 ; c1ccc(-c2ccc(-c3nc(-c4ccccc4)nc(-c4ccc(-c5ccc6ccccc6c5)c5oc6cc7ccccc7cc6c45)n3)cc2)cc1 ; c1ccc(-c2ccc3cc(-c4ccc(-c5nc(-c6ccccc6)nc(-c6ccccc6)n5)c5c4oc4cc6ccccc6cc45)ccc3c2)cc1]

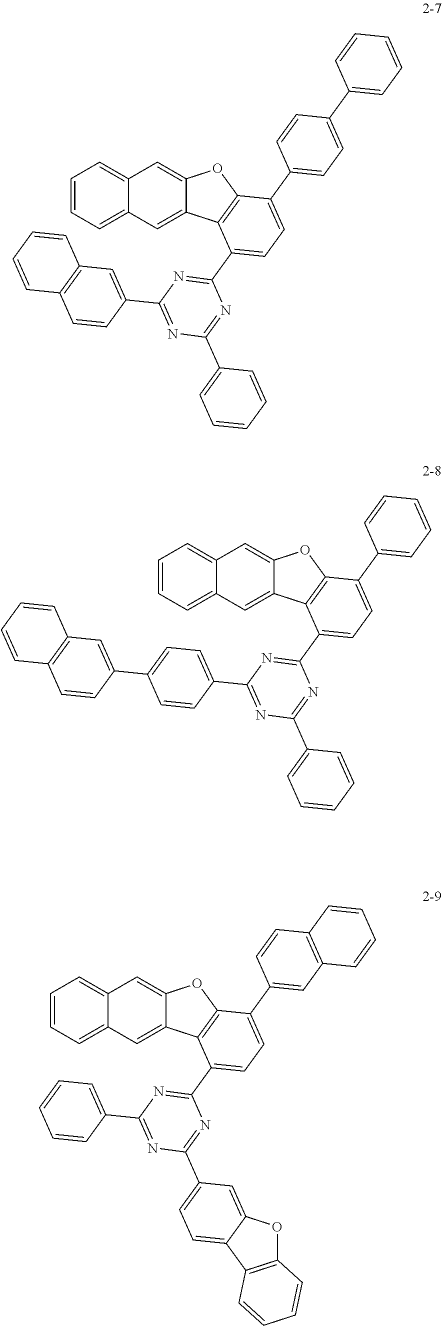 [SMILES: c1ccc(-c2ccc(-c3ccc(-c4nc(-c5ccccc5)nc(-c5ccc6ccccc6c5)n4)c4c3oc3cc5ccccc5cc34)cc2)cc1 ; c1ccc(-c2nc(-c3ccc(-c4ccc5ccccc5c4)cc3)nc(-c3ccc(-c4ccccc4)c4oc5cc6ccccc6cc5c34)n2)cc1 ; c1ccc(-c2nc(-c3ccc4c(c3)oc3ccccc34)nc(-c3ccc(-c4ccc5ccccc5c4)c4oc5cc6ccccc6cc5c34)n2)cc1]

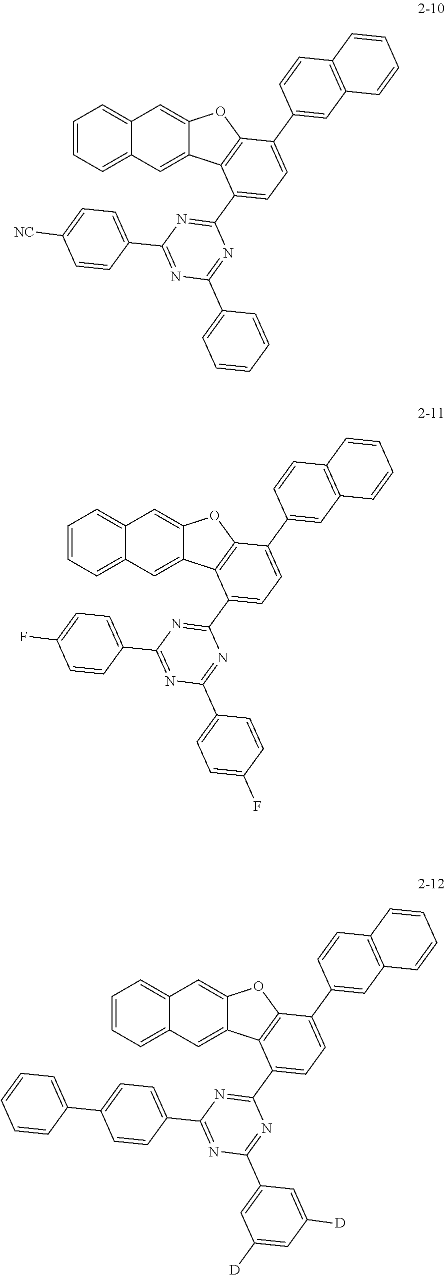 [SMILES: N#Cc1ccc(-c2nc(-c3ccccc3)nc(-c3ccc(-c4ccc5ccccc5c4)c4oc5cc6ccccc6cc5c34)n2)cc1 ; Fc1ccc(-c2nc(-c3ccc(F)cc3)nc(-c3ccc(-c4ccc5ccccc5c4)c4oc5cc6ccccc6cc5c34)n2)cc1 ; [2H]c1cc([2H])cc(-c2nc(-c3ccc(-c4ccccc4)cc3)nc(-c3ccc(-c4ccc5ccccc5c4)c4oc5cc6ccccc6cc5c34)n2)c1]

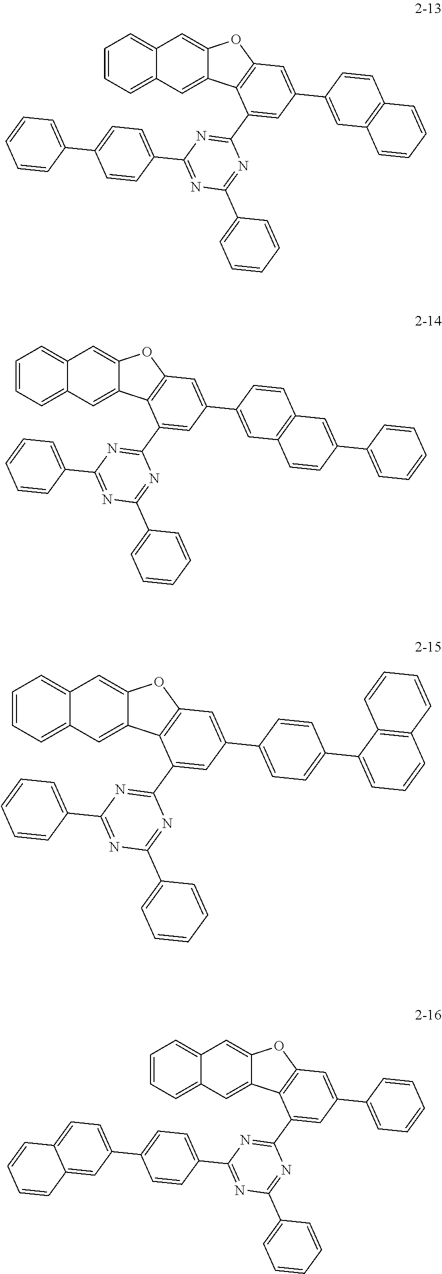 [SMILES: c1ccc(-c2ccc(-c3nc(-c4ccccc4)nc(-c4cc(-c5ccc6ccccc6c5)cc5oc6cc7ccccc7cc6c45)n3)cc2)cc1 ; c1ccc(-c2ccc3cc(-c4cc(-c5nc(-c6ccccc6)nc(-c6ccccc6)n5)c5c(c4)oc4cc6ccccc6cc45)ccc3c2)cc1 ; c1ccc(-c2nc(-c3ccccc3)nc(-c3cc(-c4ccc(-c5cccc6ccccc56)cc4)cc4oc5cc6ccccc6cc5c34)n2)cc1 ; c1ccc(-c2cc(-c3nc(-c4ccccc4)nc(-c4ccc(-c5ccc6ccccc6c5)cc4)n3)c3c(c2)oc2cc4ccccc4cc23)cc1]

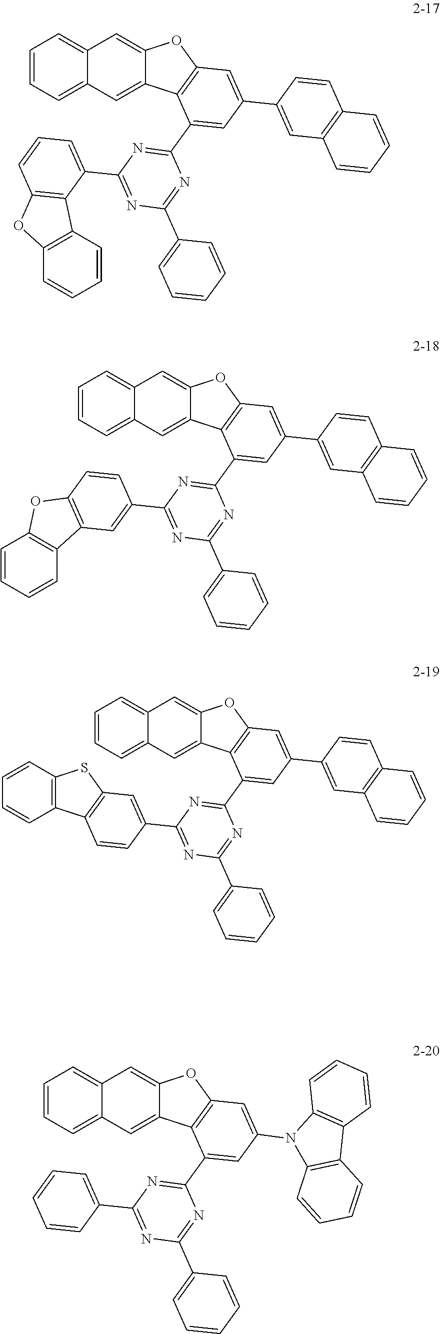 [SMILES: c1ccc(-c2nc(-c3cccc4oc5ccccc5c34)nc(-c3cc(-c4ccc5ccccc5c4)cc4oc5cc6ccccc6cc5c34)n2)cc1 ; c1ccc(-c2nc(-c3ccc4oc5ccccc5c4c3)nc(-c3cc(-c4ccc5ccccc5c4)cc4oc5cc6ccccc6cc5c34)n2)cc1 ; c1ccc(-c2nc(-c3ccc4c(c3)sc3ccccc34)nc(-c3cc(-c4ccc5ccccc5c4)cc4oc5cc6ccccc6cc5c34)n2)cc1 ; c1ccc(-c2nc(-c3ccccc3)nc(-c3cc(-n4c5ccccc5c5ccccc54)cc4oc5cc6ccccc6cc5c34)n2)cc1]

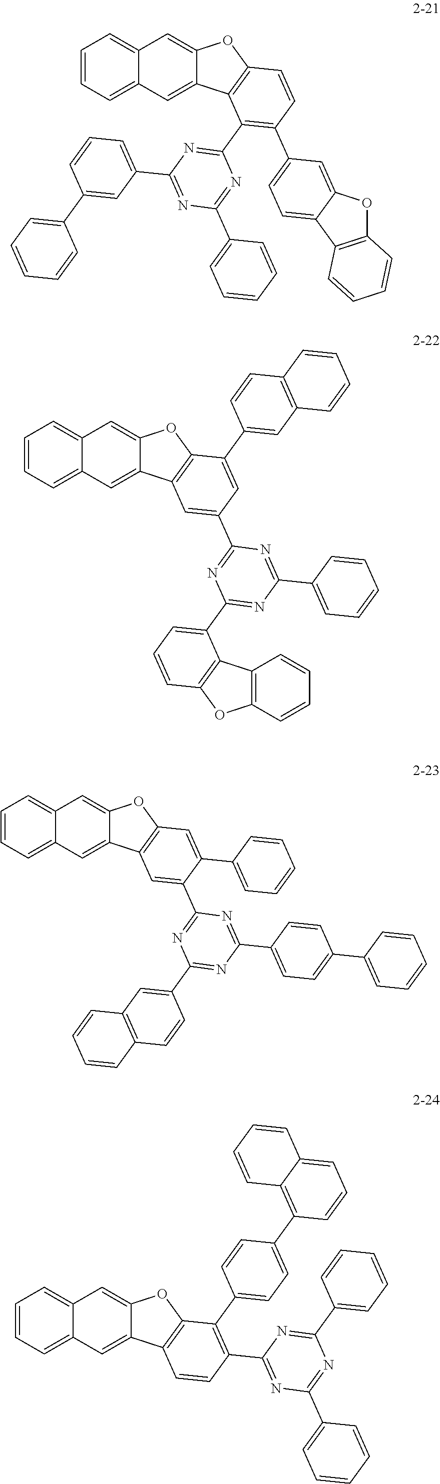 [SMILES: c1ccc(-c2cccc(-c3nc(-c4ccccc4)nc(-c4c(-c5ccc6c(c5)oc5ccccc56)ccc5oc6cc7ccccc7cc6c45)n3)c2)cc1 ; c1ccc(-c2nc(-c3cc(-c4ccc5ccccc5c4)c4oc5cc6ccccc6cc5c4c3)nc(-c3cccc4oc5ccccc5c34)n2)cc1 ; c1ccc(-c2ccc(-c3nc(-c4ccc5ccccc5c4)nc(-c4cc5c(cc4-c4ccccc4)oc4cc6ccccc6cc45)n3)cc2)cc1 ; c1ccc(-c2nc(-c3ccccc3)nc(-c3ccc4c(oc5cc6ccccc6cc54)c3-c3ccc(-c4cccc5ccccc45)cc3)n2)cc1]

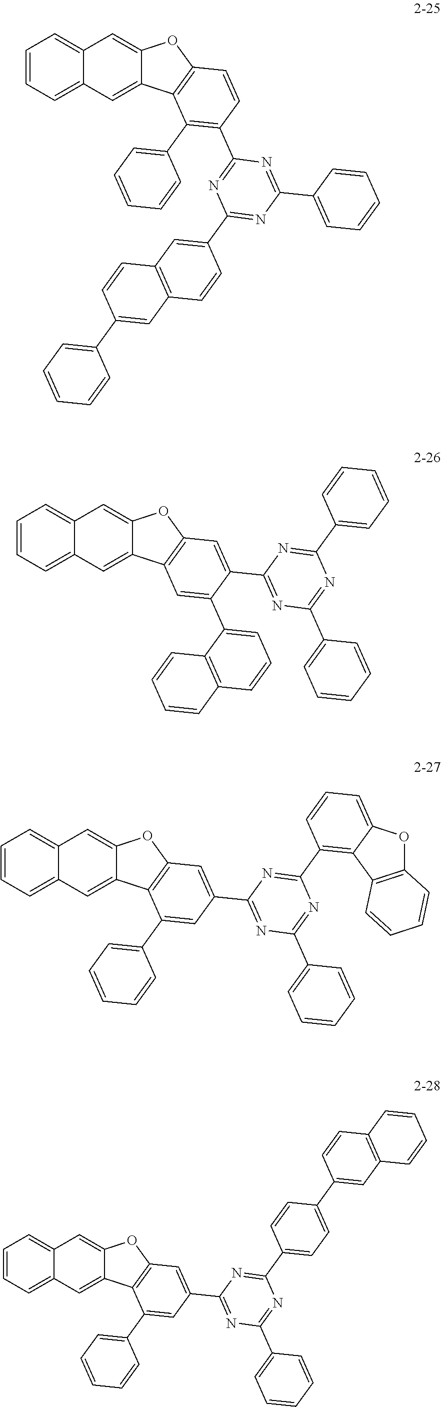 [SMILES: c1ccc(-c2ccc3cc(-c4nc(-c5ccccc5)nc(-c5ccc6oc7cc8ccccc8cc7c6c5-c5ccccc5)n4)ccc3c2)cc1 ; c1ccc(-c2nc(-c3ccccc3)nc(-c3cc4oc5cc6ccccc6cc5c4cc3-c3cccc4ccccc34)n2)cc1 ; c1ccc(-c2nc(-c3cc(-c4ccccc4)c4c(c3)oc3cc5ccccc5cc34)nc(-c3cccc4oc5ccccc5c34)n2)cc1 ; c1ccc(-c2nc(-c3ccc(-c4ccc5ccccc5c4)cc3)nc(-c3cc(-c4ccccc4)c4c(c3)oc3cc5ccccc5cc34)n2)cc1]

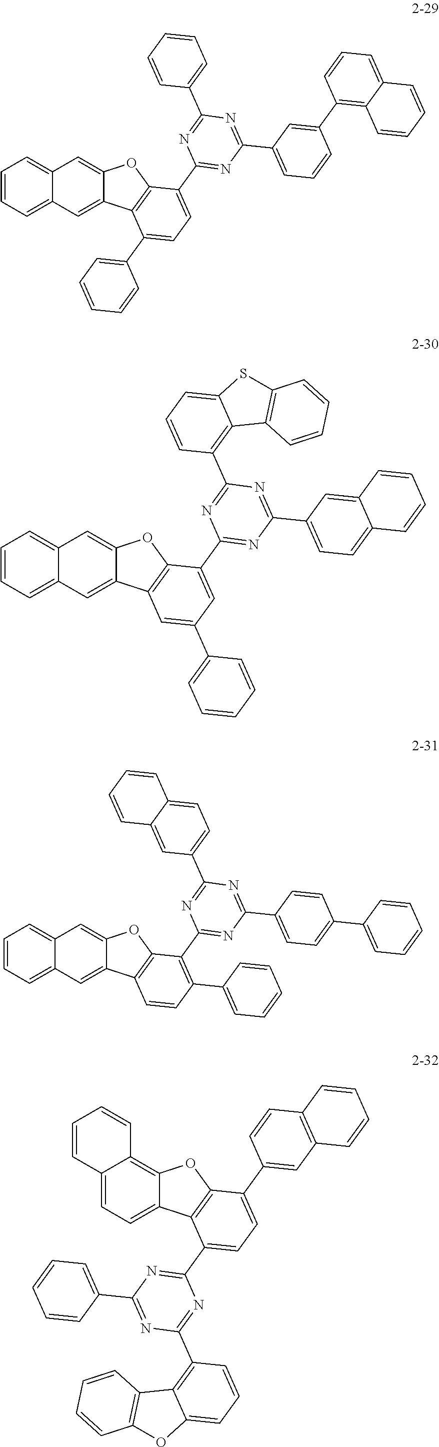 [SMILES: c1ccc(-c2nc(-c3cccc(-c4cccc5ccccc45)c3)nc(-c3ccc(-c4ccccc4)c4c3oc3cc5ccccc5cc34)n2)cc1 ; c1ccc(-c2cc(-c3nc(-c4ccc5ccccc5c4)nc(-c4cccc5sc6ccccc6c45)n3)c3oc4cc5ccccc5cc4c3c2)cc1 ; c1ccc(-c2ccc(-c3nc(-c4ccc5ccccc5c4)nc(-c4c(-c5ccccc5)ccc5c4oc4cc6ccccc6cc45)n3)cc2)cc1 ; c1ccc(-c2nc(-c3cccc4oc5ccccc5c34)nc(-c3ccc(-c4ccc5ccccc5c4)c4oc5c6ccccc6ccc5c34)n2)cc1]

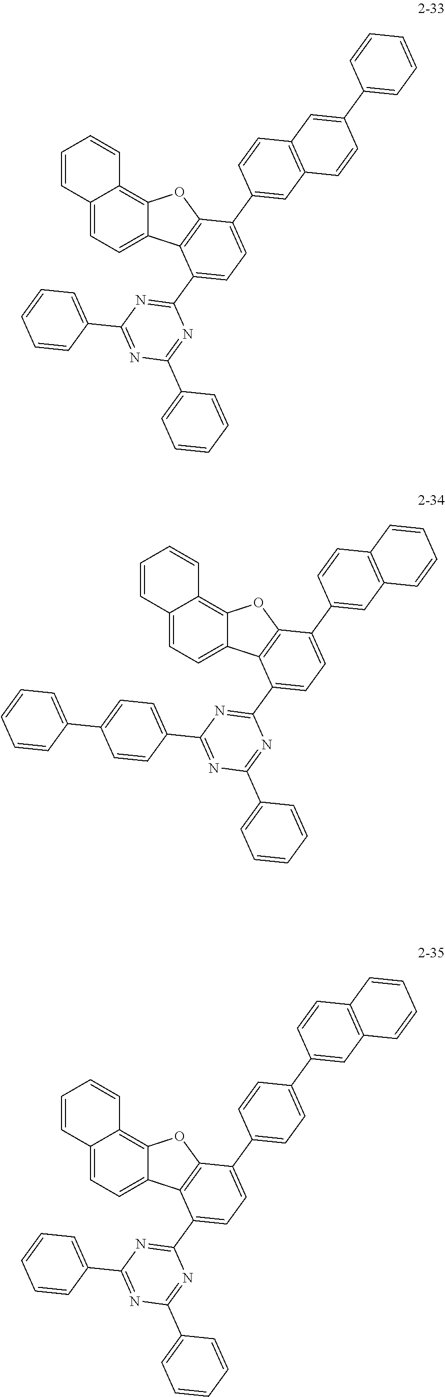 [SMILES: c1ccc(-c2ccc3cc(-c4ccc(-c5nc(-c6ccccc6)nc(-c6ccccc6)n5)c5c4oc4c6ccccc6ccc45)ccc3c2)cc1 ; c1ccc(-c2ccc(-c3nc(-c4ccccc4)nc(-c4ccc(-c5ccc6ccccc6c5)c5oc6c7ccccc7ccc6c45)n3)cc2)cc1 ; c1ccc(-c2nc(-c3ccccc3)nc(-c3ccc(-c4ccc(-c5ccc6ccccc6c5)cc4)c4oc5c6ccccc6ccc5c34)n2)cc1]

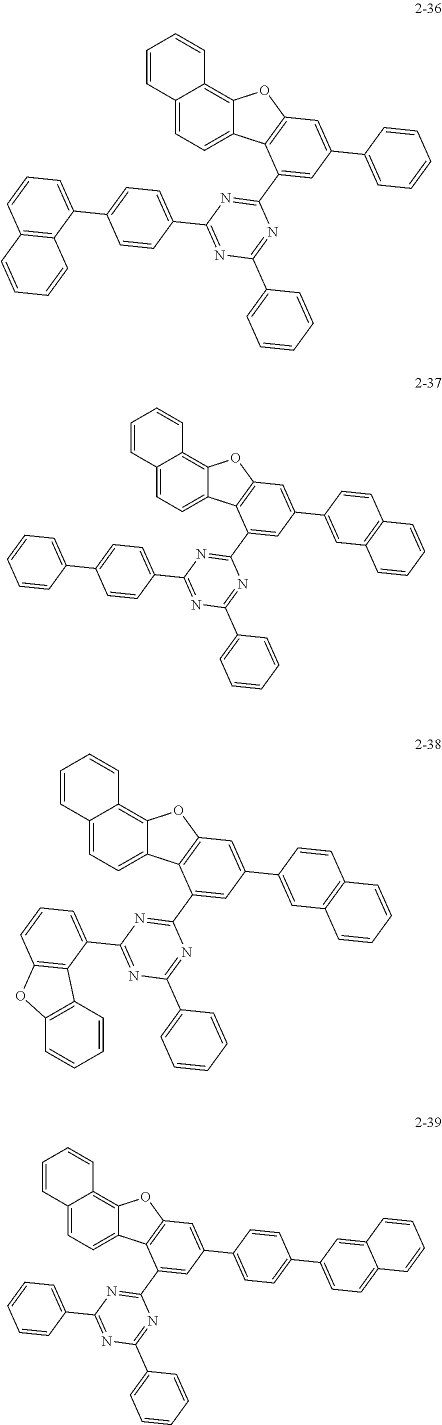 [SMILES: c1ccc(-c2cc(-c3nc(-c4ccccc4)nc(-c4ccc(-c5cccc6ccccc56)cc4)n3)c3c(c2)oc2c4ccccc4ccc23)cc1 ; c1ccc(-c2ccc(-c3nc(-c4ccccc4)nc(-c4cc(-c5ccc6ccccc6c5)cc5oc6c7ccccc7ccc6c45)n3)cc2)cc1 ; c1ccc(-c2nc(-c3cccc4oc5ccccc5c34)nc(-c3cc(-c4ccc5ccccc5c4)cc4oc5c6ccccc6ccc5c34)n2)cc1 ; c1ccc(-c2nc(-c3ccccc3)nc(-c3cc(-c4ccc(-c5ccc6ccccc6c5)cc4)cc4oc5c6ccccc6ccc5c34)n2)cc1]

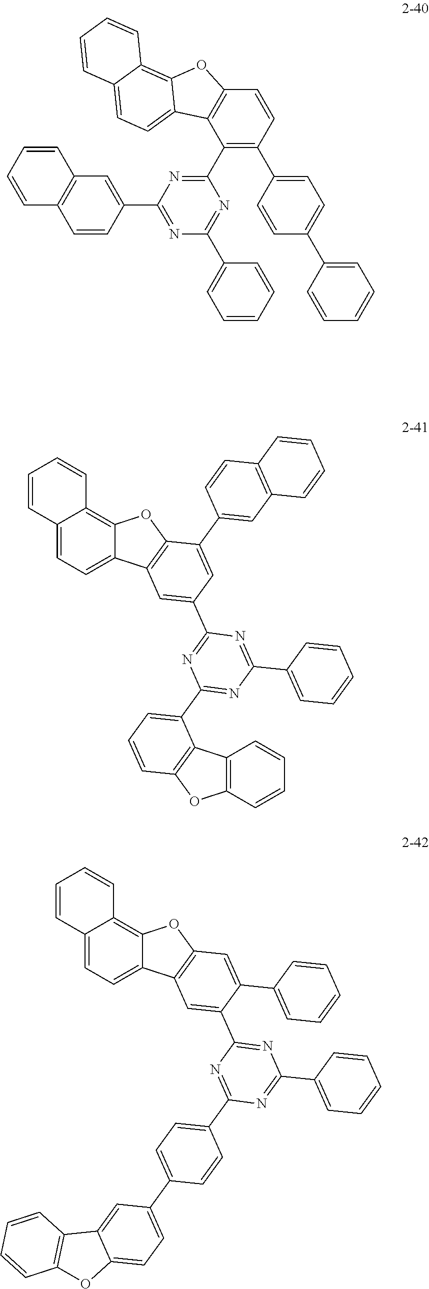 [SMILES: c1ccc(-c2ccc(-c3ccc4oc5c6ccccc6ccc5c4c3-c3nc(-c4ccccc4)nc(-c4ccc5ccccc5c4)n3)cc2)cc1 ; c1ccc(-c2nc(-c3cc(-c4ccc5ccccc5c4)c4oc5c6ccccc6ccc5c4c3)nc(-c3cccc4oc5ccccc5c34)n2)cc1 ; c1ccc(-c2nc(-c3ccc(-c4ccc5oc6ccccc6c5c4)cc3)nc(-c3cc4c(cc3-c3ccccc3)oc3c5ccccc5ccc43)n2)cc1]

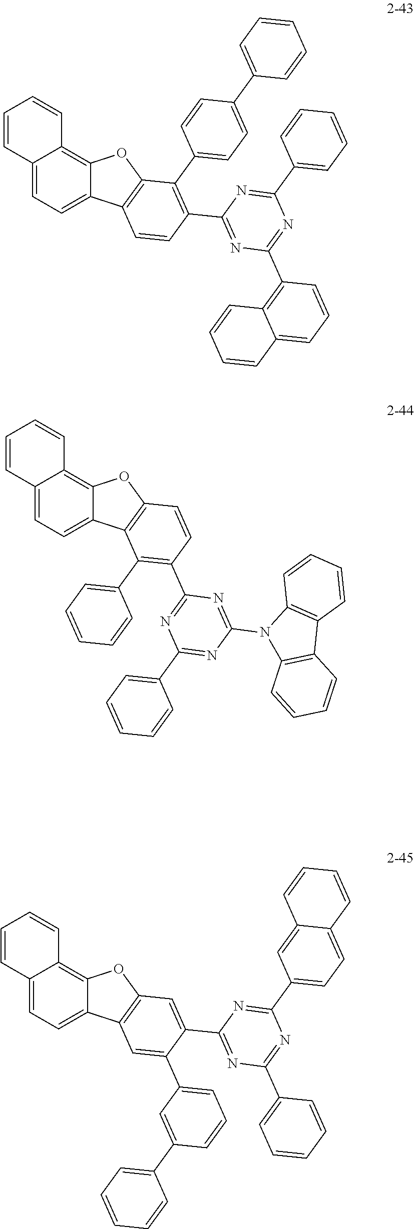 [SMILES: c1ccc(-c2ccc(-c3c(-c4nc(-c5ccccc5)nc(-c5cccc6ccccc56)n4)ccc4c3oc3c5ccccc5ccc43)cc2)cc1 ; c1ccc(-c2nc(-c3ccc4oc5c6ccccc6ccc5c4c3-c3ccccc3)nc(-n3c4ccccc4c4ccccc43)n2)cc1 ; c1ccc(-c2cccc(-c3cc4c(cc3-c3nc(-c5ccccc5)nc(-c5ccc6ccccc6c5)n3)oc3c5ccccc5ccc43)c2)cc1]

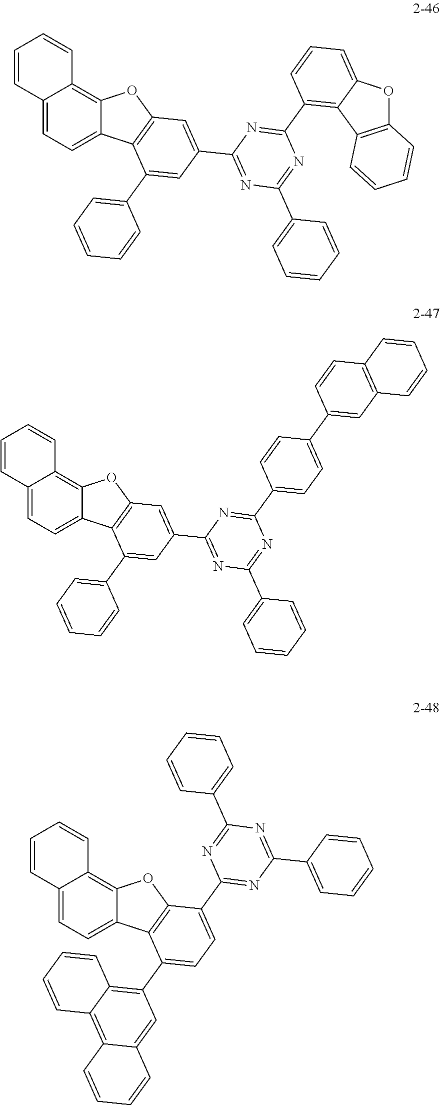 [SMILES: c1ccc(-c2nc(-c3cc(-c4ccccc4)c4c(c3)oc3c5ccccc5ccc34)nc(-c3cccc4oc5ccccc5c34)n2)cc1 ; c1ccc(-c2nc(-c3ccc(-c4ccc5ccccc5c4)cc3)nc(-c3cc(-c4ccccc4)c4c(c3)oc3c5ccccc5ccc34)n2)cc1 ; c1ccc(-c2nc(-c3ccccc3)nc(-c3ccc(-c4cc5ccccc5c5ccccc45)c4c3oc3c5ccccc5ccc34)n2)cc1]

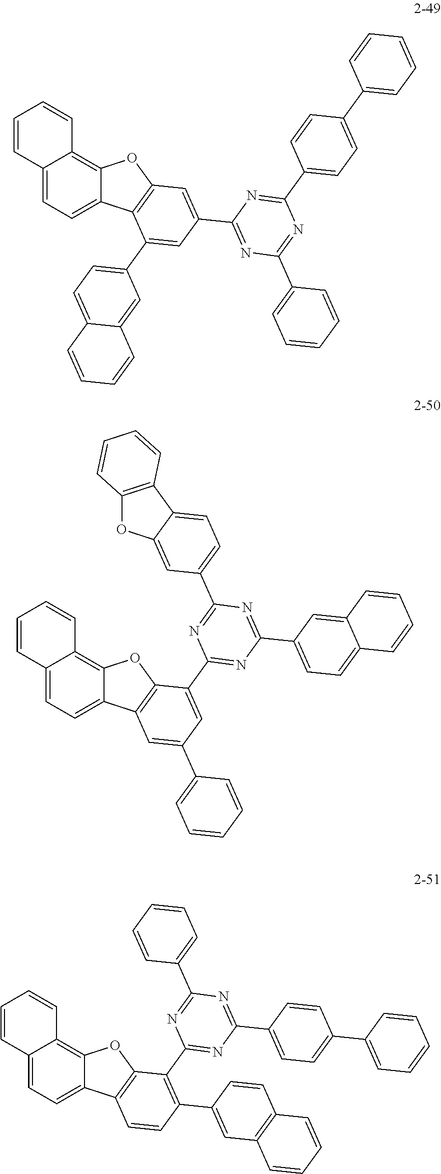 [SMILES: c1ccc(-c2ccc(-c3nc(-c4ccccc4)nc(-c4cc(-c5ccc6ccccc6c5)c5c(c4)oc4c6ccccc6ccc45)n3)cc2)cc1 ; c1ccc(-c2cc(-c3nc(-c4ccc5ccccc5c4)nc(-c4ccc5c(c4)oc4ccccc45)n3)c3oc4c5ccccc5ccc4c3c2)cc1 ; c1ccc(-c2ccc(-c3nc(-c4ccccc4)nc(-c4c(-c5ccc6ccccc6c5)ccc5c4oc4c6ccccc6ccc54)n3)cc2)cc1]

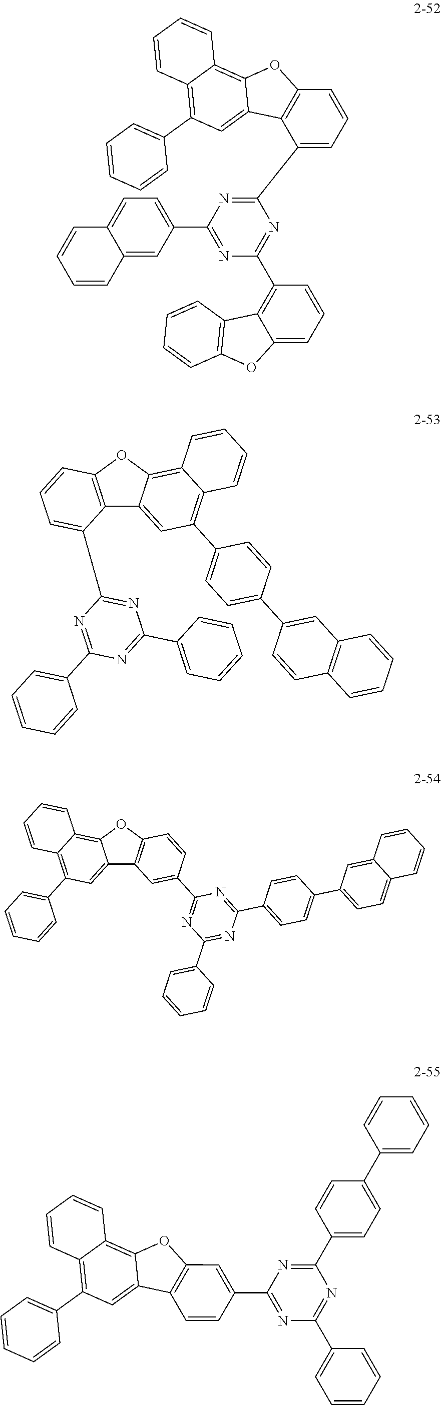 [SMILES: c1ccc(-c2cc3c(oc4cccc(-c5nc(-c6ccc7ccccc7c6)nc(-c6cccc7oc8ccccc8c67)n5)c43)c3ccccc23)cc1 ; c1ccc(-c2nc(-c3ccccc3)nc(-c3cccc4oc5c6ccccc6c(-c6ccc(-c7ccc8ccccc8c7)cc6)cc5c34)n2)cc1 ; c1ccc(-c2nc(-c3ccc(-c4ccc5ccccc5c4)cc3)nc(-c3ccc4oc5c6ccccc6c(-c6ccccc6)cc5c4c3)n2)cc1 ; c1ccc(-c2ccc(-c3nc(-c4ccccc4)nc(-c4ccc5c(c4)oc4c6ccccc6c(-c6ccccc6)cc54)n3)cc2)cc1]

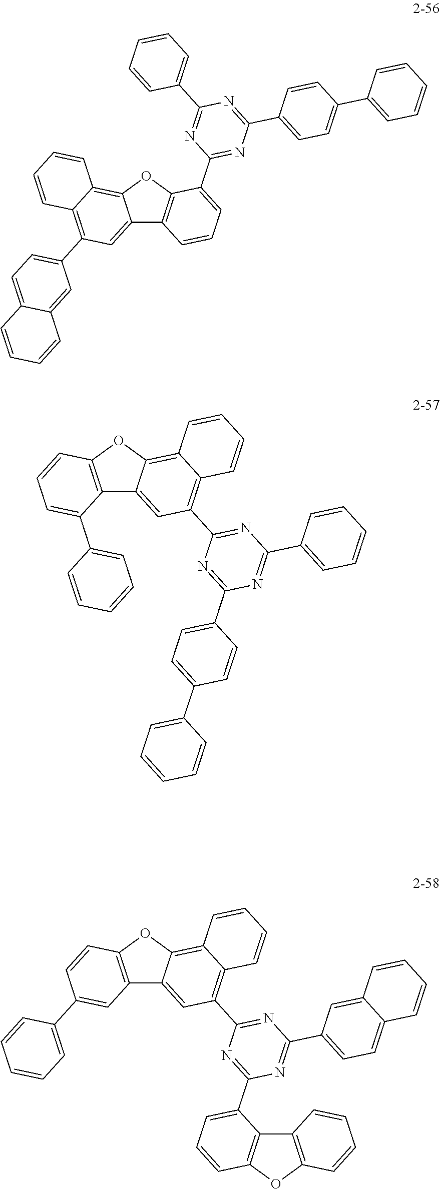 [SMILES: c1ccc(-c2ccc(-c3nc(-c4ccccc4)nc(-c4cccc5c4oc4c6ccccc6c(-c6ccc7ccccc7c6)cc54)n3)cc2)cc1 ; c1ccc(-c2ccc(-c3nc(-c4ccccc4)nc(-c4cc5c(oc6cccc(-c7ccccc7)c65)c5ccccc45)n3)cc2)cc1 ; c1ccc(-c2ccc3oc4c5ccccc5c(-c5nc(-c6ccc7ccccc7c6)nc(-c6cccc7oc8ccccc8c67)n5)cc4c3c2)cc1]

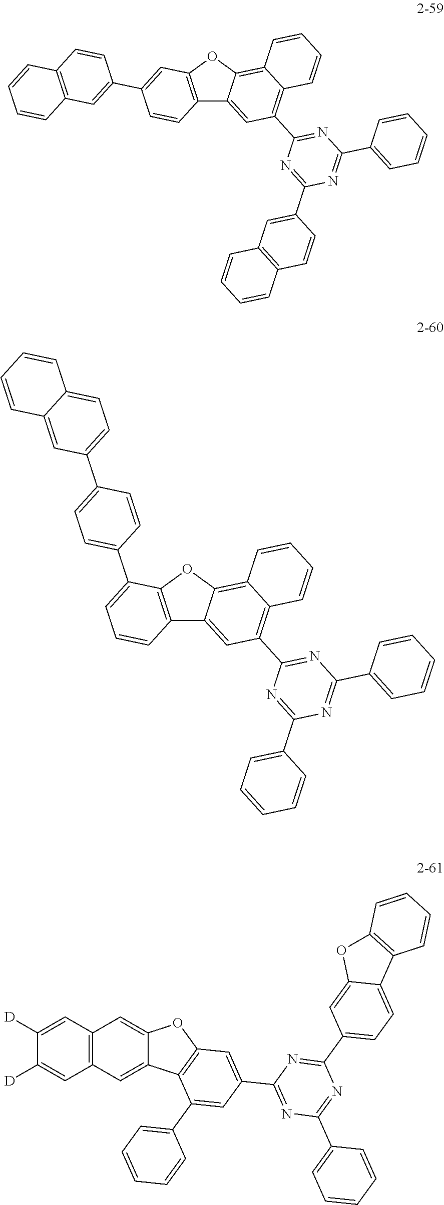 [SMILES: c1ccc(-c2nc(-c3ccc4ccccc4c3)nc(-c3cc4c5ccc(-c6ccc7ccccc7c6)cc5oc4c4ccccc34)n2)cc1 ; c1ccc(-c2nc(-c3ccccc3)nc(-c3cc4c5cccc(-c6ccc(-c7ccc8ccccc8c7)cc6)c5oc4c4ccccc34)n2)cc1 ; [2H]c1cc2cc3oc4cc(-c5nc(-c6ccccc6)nc(-c6ccc7c(c6)oc6ccccc67)n5)cc(-c5ccccc5)c4c3cc2cc1[2H]]

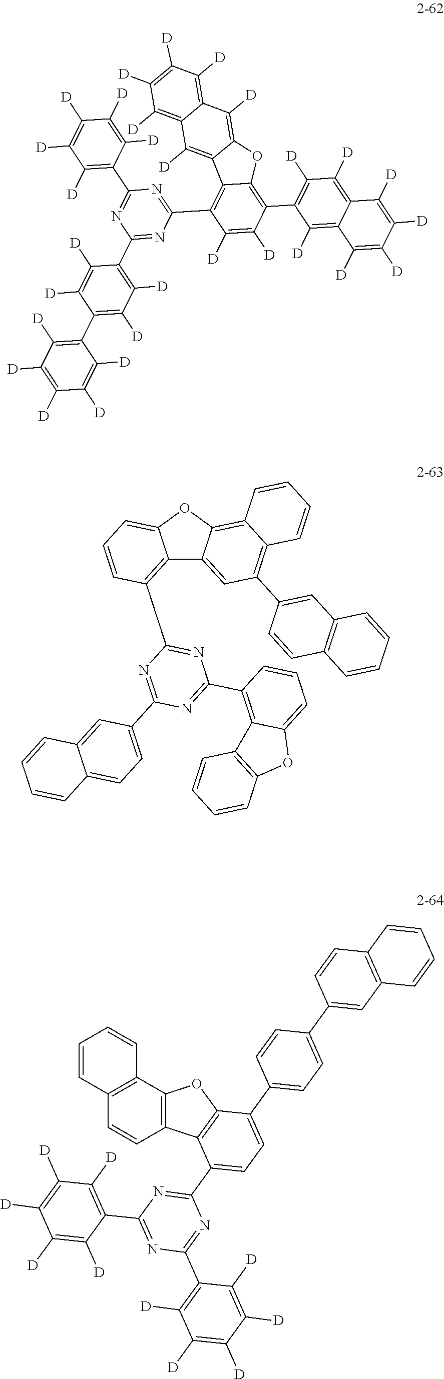 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-c4c([2H])c([2H])c5c([2H])c([2H])c([2H])c([2H])c5c4[2H])c4oc5c([2H])c6c([2H])c([2H])c([2H])c([2H])c6c([2H])c5c34)n2)c([2H])c1[2H] ; c1ccc2cc(-c3nc(-c4cccc5oc6ccccc6c45)nc(-c4cccc5oc6c7ccccc7c(-c7ccc8ccccc8c7)cc6c45)n3)ccc2c1 ; [2H]c1c([2H])c([2H])c(-c2nc(-c3c([2H])c([2H])c([2H])c([2H])c3[2H])nc(-c3ccc(-c4ccc(-c5ccc6ccccc6c5)cc4)c4oc5c6ccccc6ccc5c34)n2)c([2H])c1[2H]]

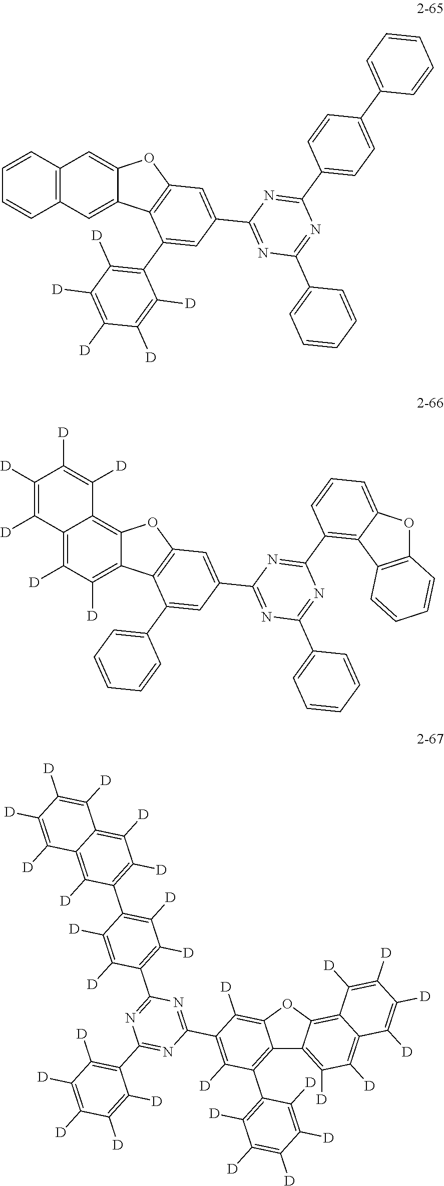 [SMILES: [2H]c1c([2H])c([2H])c(-c2cc(-c3nc(-c4ccccc4)nc(-c4ccc(-c5ccccc5)cc4)n3)cc3oc4cc5ccccc5cc4c23)c([2H])c1[2H] ; [2H]c1c([2H])c([2H])c2c(c1[2H])c([2H])c([2H])c1c2oc2cc(-c3nc(-c4ccccc4)nc(-c4cccc5oc6ccccc6c45)n3)cc(-c3ccccc3)c21 ; [2H]c1c([2H])c([2H])c(-c2nc(-c3c([2H])c([2H])c(-c4c([2H])c([2H])c5c([2H])c([2H])c([2H])c([2H])c5c4[2H])c([2H])c3[2H])nc(-c3c([2H])c(-c4c([2H])c([2H])c([2H])c([2H])c4[2H])c4c(oc5c6c([2H])c([2H])c([2H])c([2H])c6c([2H])c([2H])c54)c3[2H])n2)c([2H])c1[2H]]

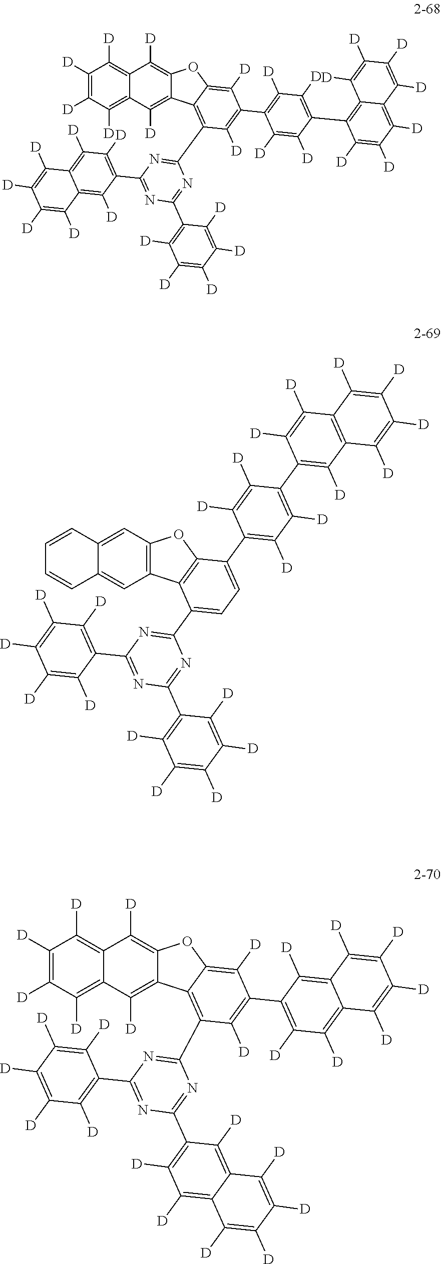

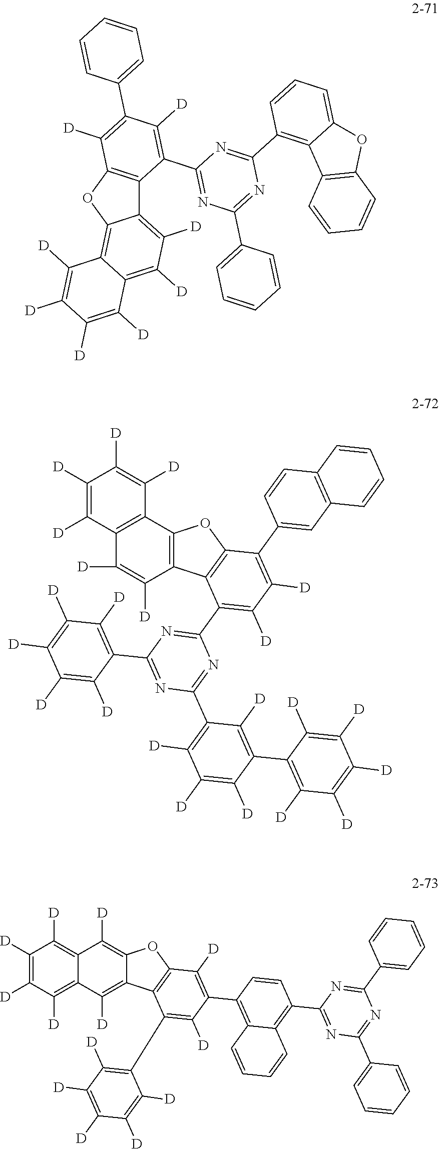

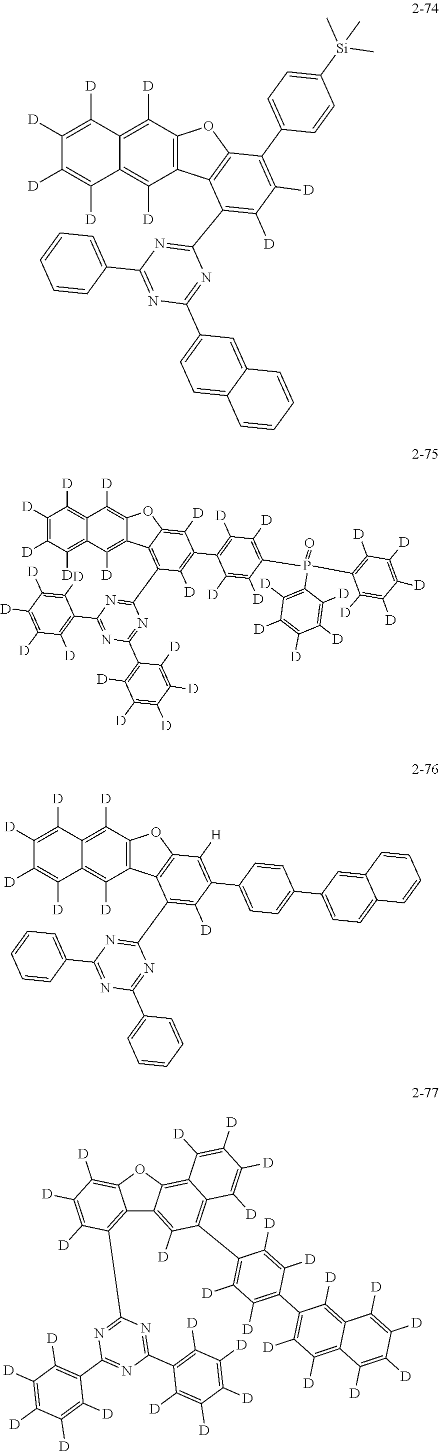

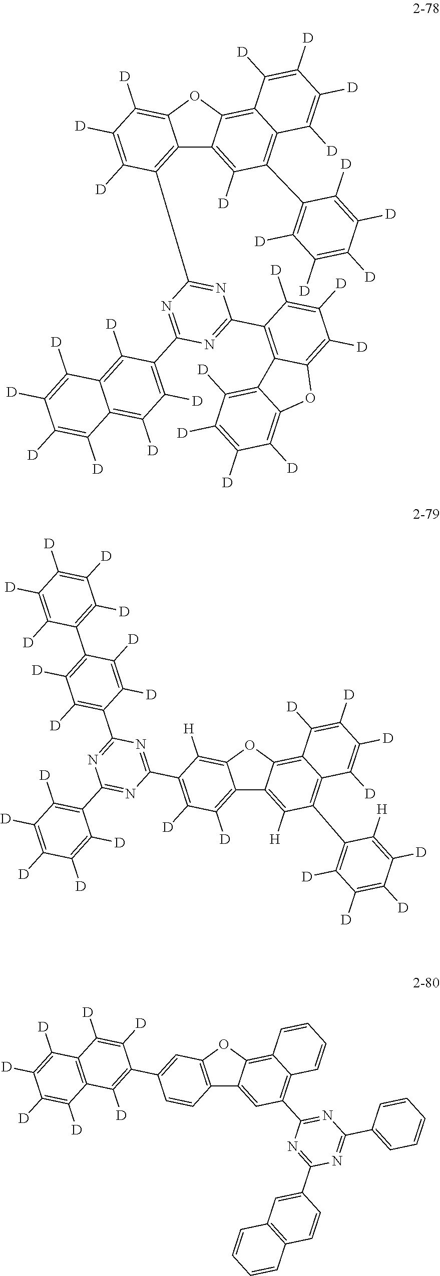

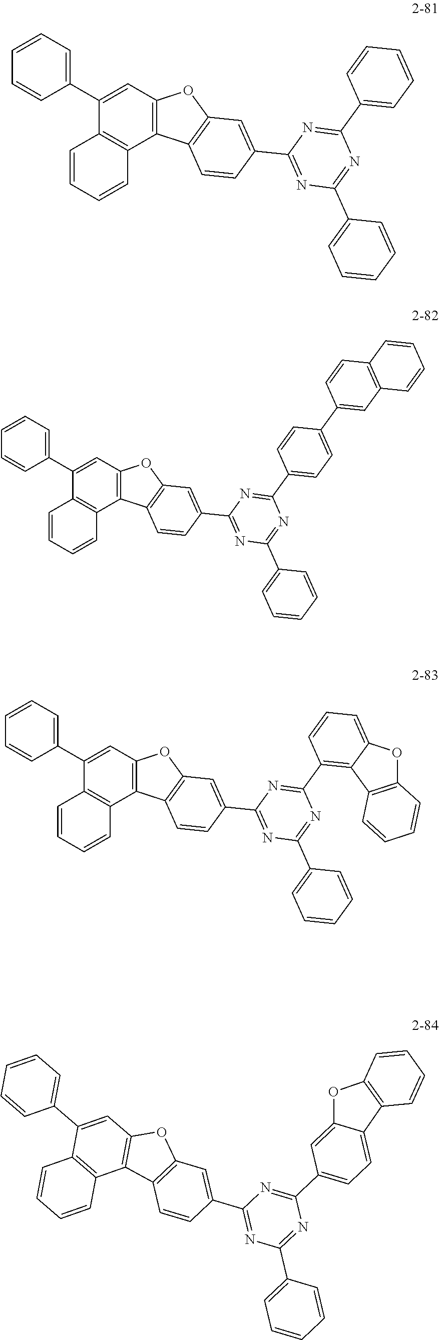 [SMILES: c1ccc(-c2nc(-c3ccccc3)nc(-c3ccc4c(c3)oc3cc(-c5ccccc5)c5ccccc5c34)n2)cc1 ; c1ccc(-c2nc(-c3ccc(-c4ccc5ccccc5c4)cc3)nc(-c3ccc4c(c3)oc3cc(-c5ccccc5)c5ccccc5c34)n2)cc1 ; c1ccc(-c2nc(-c3ccc4c(c3)oc3cc(-c5ccccc5)c5ccccc5c34)nc(-c3cccc4oc5ccccc5c34)n2)cc1 ; c1ccc(-c2nc(-c3ccc4c(c3)oc3ccccc34)nc(-c3ccc4c(c3)oc3cc(-c5ccccc5)c5ccccc5c34)n2)cc1]

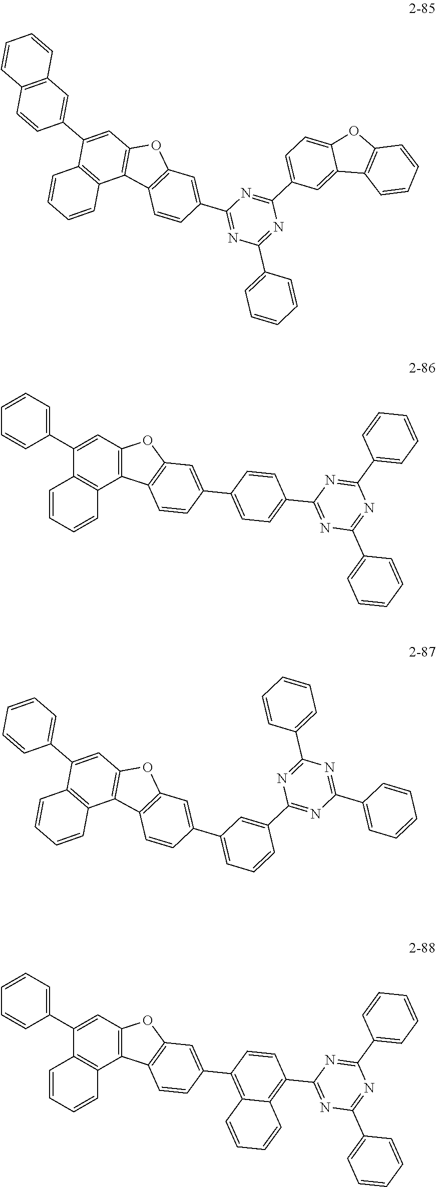 [SMILES: c1ccc(-c2nc(-c3ccc4c(c3)oc3cc(-c5ccc6ccccc6c5)c5ccccc5c34)nc(-c3ccc4oc5ccccc5c4c3)n2)cc1 ; c1ccc(-c2nc(-c3ccccc3)nc(-c3ccc(-c4ccc5c(c4)oc4cc(-c6ccccc6)c6ccccc6c45)cc3)n2)cc1 ; c1ccc(-c2nc(-c3ccccc3)nc(-c3cccc(-c4ccc5c(c4)oc4cc(-c6ccccc6)c6ccccc6c45)c3)n2)cc1 ; c1ccc(-c2nc(-c3ccccc3)nc(-c3ccc(-c4ccc5c(c4)oc4cc(-c6ccccc6)c6ccccc6c45)c4ccccc34)n2)cc1]

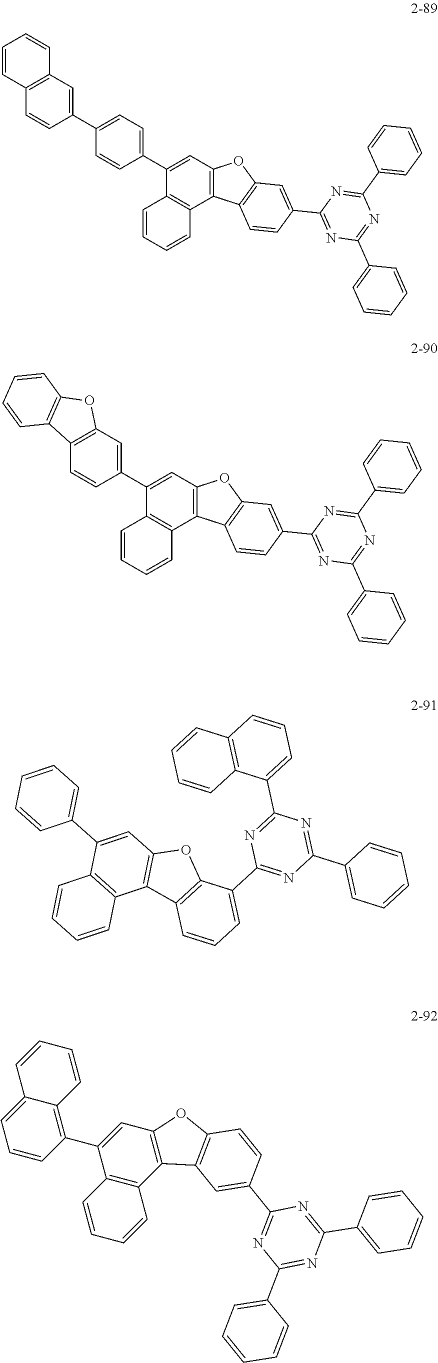 [SMILES: c1ccc(-c2nc(-c3ccccc3)nc(-c3ccc4c(c3)oc3cc(-c5ccc(-c6ccc7ccccc7c6)cc5)c5ccccc5c34)n2)cc1 ; c1ccc(-c2nc(-c3ccccc3)nc(-c3ccc4c(c3)oc3cc(-c5ccc6c(c5)oc5ccccc56)c5ccccc5c34)n2)cc1 ; c1ccc(-c2nc(-c3cccc4ccccc34)nc(-c3cccc4c3oc3cc(-c5ccccc5)c5ccccc5c34)n2)cc1 ; c1ccc(-c2nc(-c3ccccc3)nc(-c3ccc4oc5cc(-c6cccc7ccccc67)c6ccccc6c5c4c3)n2)cc1]

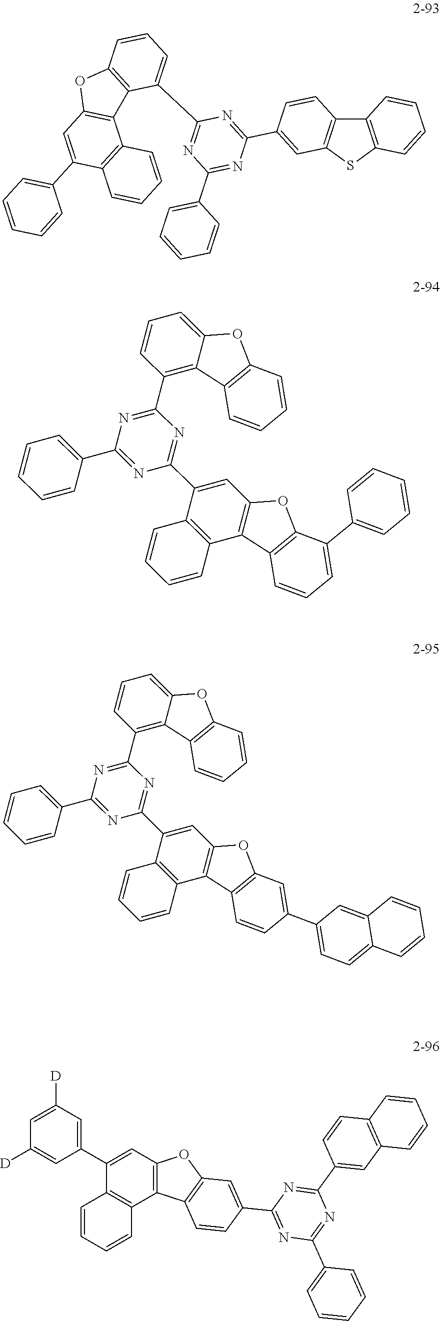 [SMILES: c1ccc(-c2nc(-c3ccc4c(c3)sc3ccccc34)nc(-c3cccc4oc5cc(-c6ccccc6)c6ccccc6c5c34)n2)cc1 ; c1ccc(-c2nc(-c3cc4oc5c(-c6ccccc6)cccc5c4c4ccccc34)nc(-c3cccc4oc5ccccc5c34)n2)cc1 ; c1ccc(-c2nc(-c3cc4oc5cc(-c6ccc7ccccc7c6)ccc5c4c4ccccc34)nc(-c3cccc4oc5ccccc5c34)n2)cc1 ; [2H]c1cc([2H])cc(-c2cc3oc4cc(-c5nc(-c6ccccc6)nc(-c6ccc7ccccc7c6)n5)ccc4c3c3ccccc23)c1]

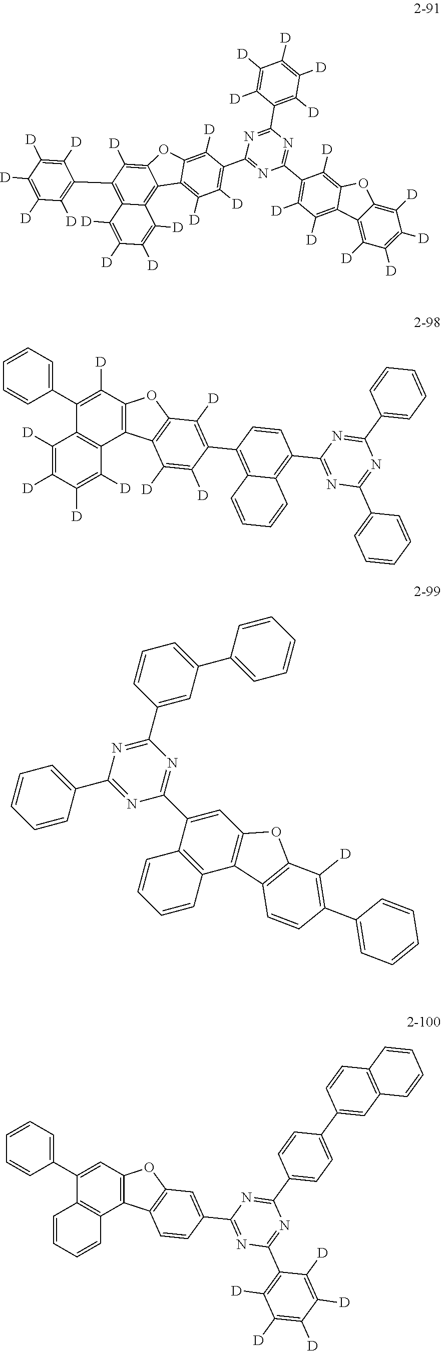

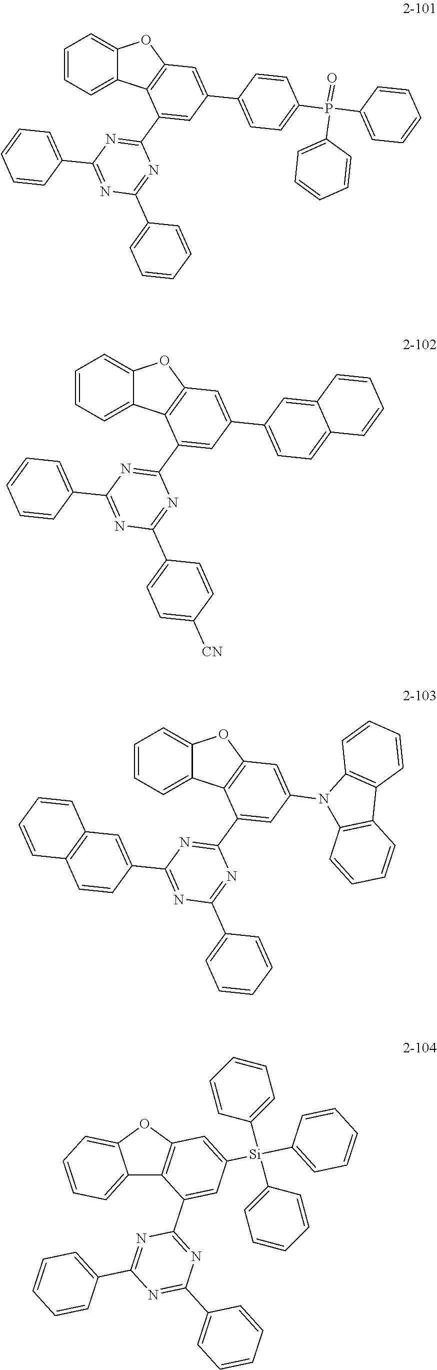 [SMILES: O=P(c1ccccc1)(c1ccccc1)c1ccc(-c2cc(-c3nc(-c4ccccc4)nc(-c4ccccc4)n3)c3c(c2)oc2ccccc23)cc1 ; N#Cc1ccc(-c2nc(-c3ccccc3)nc(-c3cc(-c4ccc5ccccc5c4)cc4oc5ccccc5c34)n2)cc1 ; c1ccc(-c2nc(-c3ccc4ccccc4c3)nc(-c3cc(-n4c5ccccc5c5ccccc54)cc4oc5ccccc5c34)n2)cc1 ; c1ccc(-c2nc(-c3ccccc3)nc(-c3cc([Si](c4ccccc4)(c4ccccc4)c4ccccc4)cc4oc5ccccc5c34)n2)cc1]

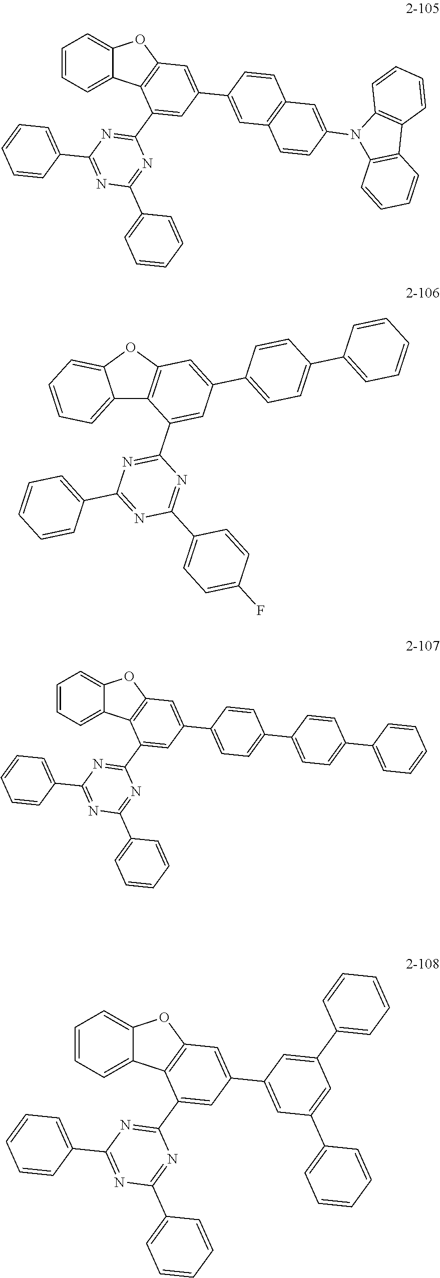 [SMILES: c1ccc(-c2nc(-c3ccccc3)nc(-c3cc(-c4ccc5cc(-n6c7ccccc7c7ccccc76)ccc5c4)cc4oc5ccccc5c34)n2)cc1 ; Fc1ccc(-c2nc(-c3ccccc3)nc(-c3cc(-c4ccc(-c5ccccc5)cc4)cc4oc5ccccc5c34)n2)cc1 ; c1ccc(-c2ccc(-c3ccc(-c4cc(-c5nc(-c6ccccc6)nc(-c6ccccc6)n5)c5c(c4)oc4ccccc45)cc3)cc2)cc1 ; c1ccc(-c2cc(-c3ccccc3)cc(-c3cc(-c4nc(-c5ccccc5)nc(-c5ccccc5)n4)c4c(c3)oc3ccccc34)c2)cc1]

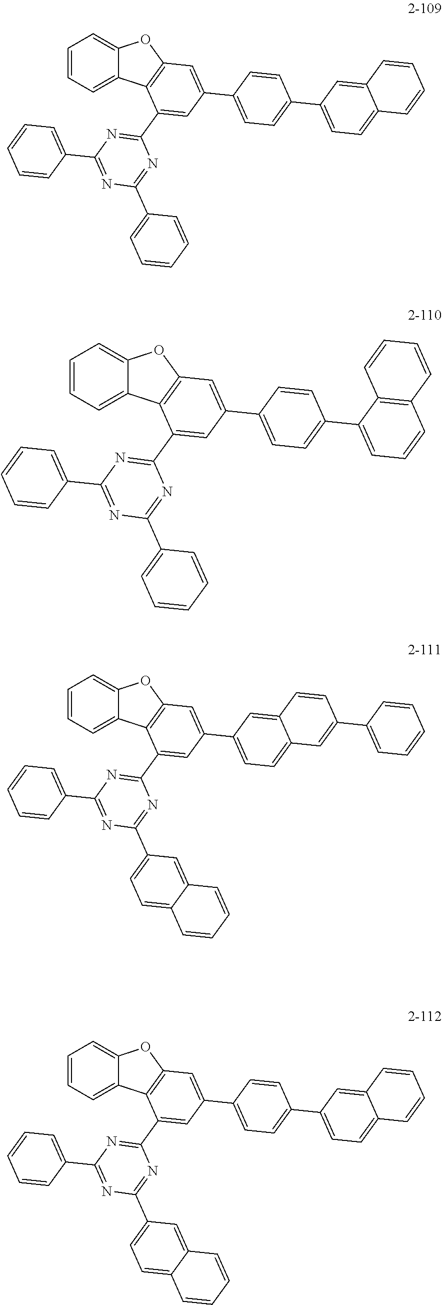 [SMILES: c1ccc(-c2nc(-c3ccccc3)nc(-c3cc(-c4ccc(-c5ccc6ccccc6c5)cc4)cc4oc5ccccc5c34)n2)cc1 ; c1ccc(-c2nc(-c3ccccc3)nc(-c3cc(-c4ccc(-c5cccc6ccccc56)cc4)cc4oc5ccccc5c34)n2)cc1 ; c1ccc(-c2ccc3cc(-c4cc(-c5nc(-c6ccccc6)nc(-c6ccc7ccccc7c6)n5)c5c(c4)oc4ccccc45)ccc3c2)cc1 ; c1ccc(-c2nc(-c3ccc4ccccc4c3)nc(-c3cc(-c4ccc(-c5ccc6ccccc6c5)cc4)cc4oc5ccccc5c34)n2)cc1]

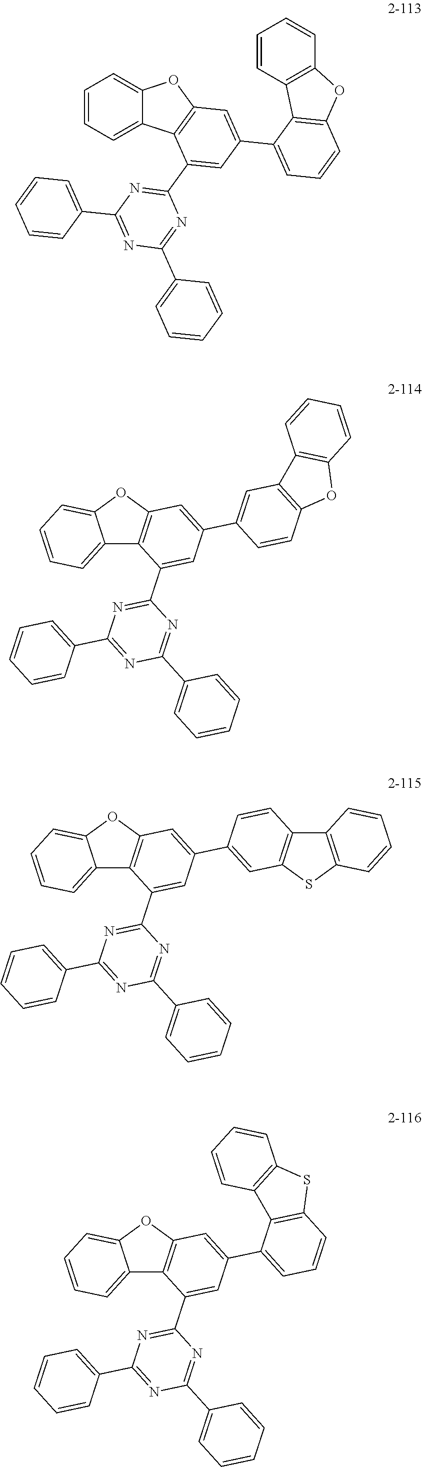 [SMILES: c1ccc(-c2nc(-c3ccccc3)nc(-c3cc(-c4cccc5oc6ccccc6c45)cc4oc5ccccc5c34)n2)cc1 ; c1ccc(-c2nc(-c3ccccc3)nc(-c3cc(-c4ccc5oc6ccccc6c5c4)cc4oc5ccccc5c34)n2)cc1 ; c1ccc(-c2nc(-c3ccccc3)nc(-c3cc(-c4ccc5c(c4)sc4ccccc45)cc4oc5ccccc5c34)n2)cc1 ; c1ccc(-c2nc(-c3ccccc3)nc(-c3cc(-c4cccc5sc6ccccc6c45)cc4oc5ccccc5c34)n2)cc1]

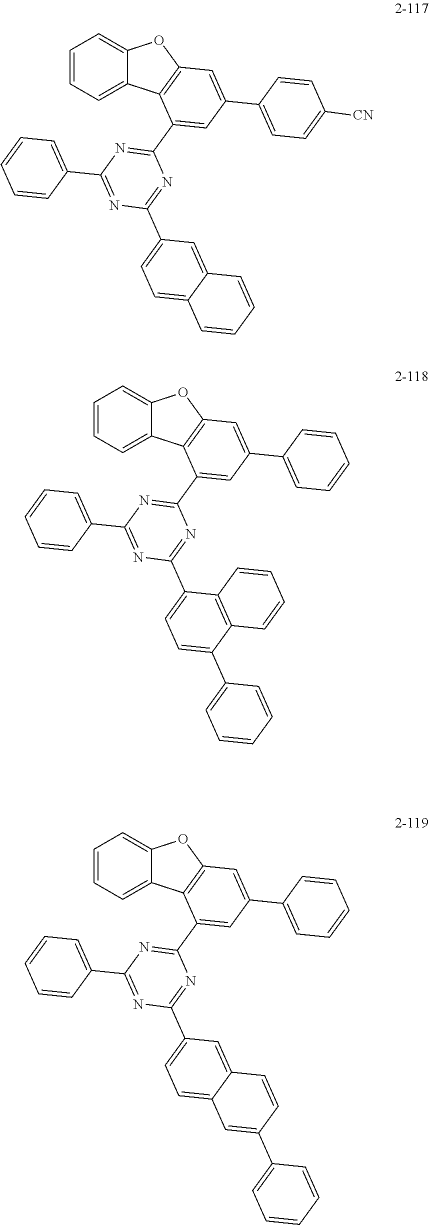 [SMILES: N#Cc1ccc(-c2cc(-c3nc(-c4ccccc4)nc(-c4ccc5ccccc5c4)n3)c3c(c2)oc2ccccc23)cc1 ; c1ccc(-c2cc(-c3nc(-c4ccccc4)nc(-c4ccc(-c5ccccc5)c5ccccc45)n3)c3c(c2)oc2ccccc23)cc1 ; c1ccc(-c2ccc3cc(-c4nc(-c5ccccc5)nc(-c5cc(-c6ccccc6)cc6oc7ccccc7c56)n4)ccc3c2)cc1]

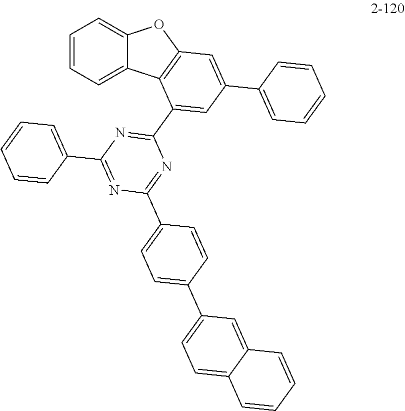 [SMILES: c1ccc(-c2cc(-c3nc(-c4ccccc4)nc(-c4ccc(-c5ccc6ccccc6c5)cc4)n3)c3c(c2)oc2ccccc23)cc1]

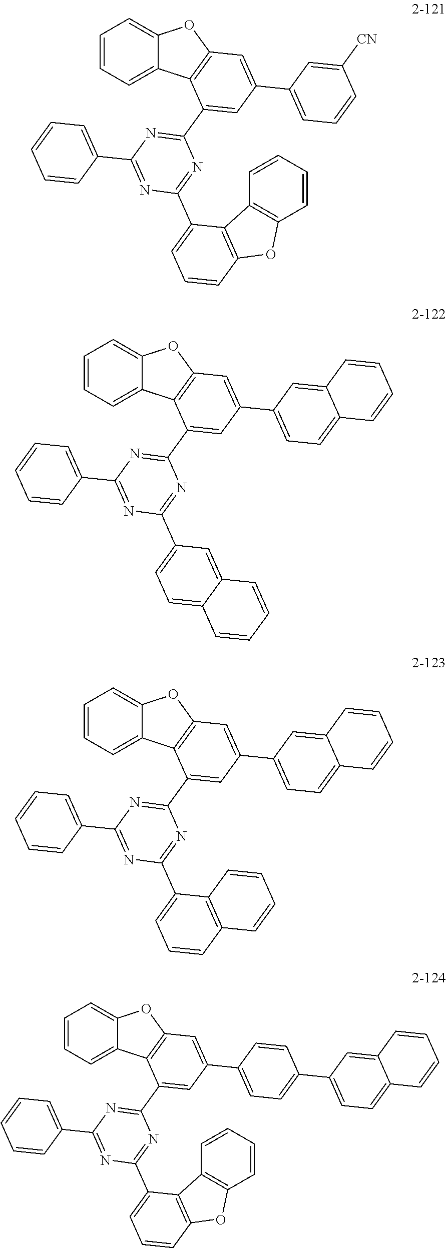 [SMILES: N#Cc1cccc(-c2cc(-c3nc(-c4ccccc4)nc(-c4cccc5oc6ccccc6c45)n3)c3c(c2)oc2ccccc23)c1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cccc4ccccc34)nc(-c3cc(-c4ccc5ccccc5c4)cc4oc5ccccc5c34)n2)cc1 ; c1ccc(-c2nc(-c3cccc4oc5ccccc5c34)nc(-c3cc(-c4ccc(-c5ccc6ccccc6c5)cc4)cc4oc5ccccc5c34)n2)cc1]

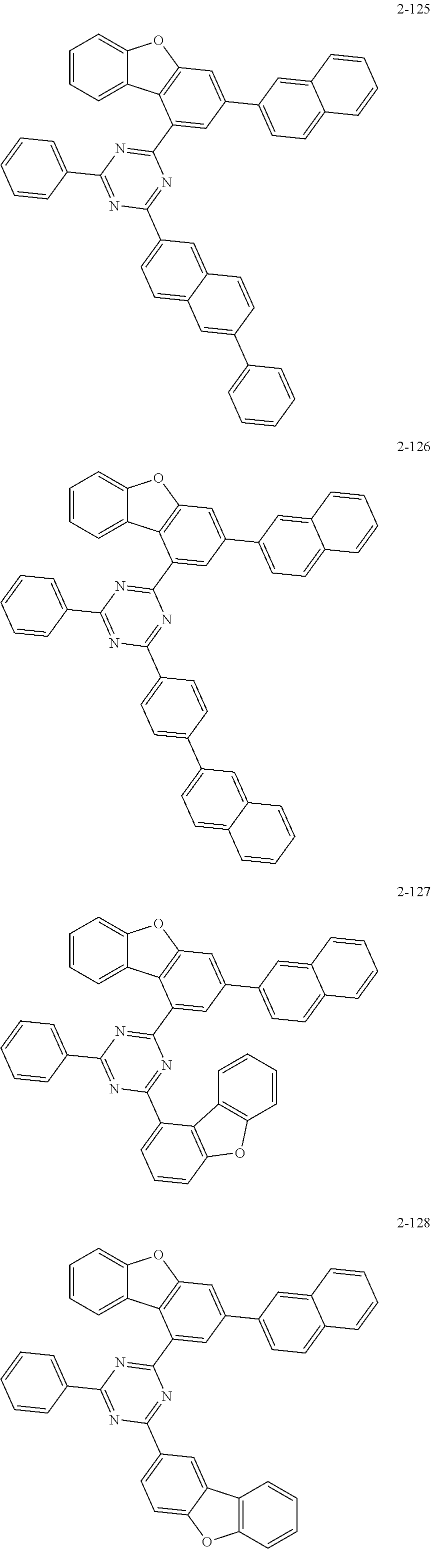 [SMILES: c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3ccc4oc5ccccc5c4c3)nc(-c3cc(-c4ccc5ccccc5c4)cc4oc5ccccc5c34)n2)cc1]

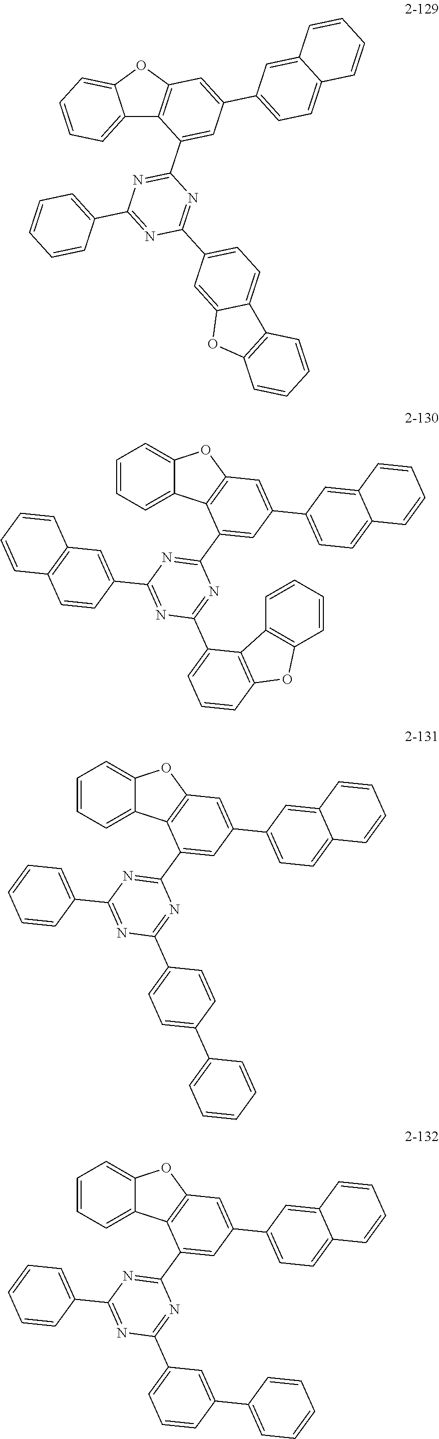 [SMILES: c1ccc(-c2nc(-c3ccc4c(c3)oc3ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5cccc6oc7ccccc7c56)n4)c4c(c3)oc3ccccc34)ccc2c1 ; c1ccc(-c2ccc(-c3nc(-c4ccccc4)nc(-c4cc(-c5ccc6ccccc6c5)cc5oc6ccccc6c45)n3)cc2)cc1 ; c1ccc(-c2cccc(-c3nc(-c4ccccc4)nc(-c4cc(-c5ccc6ccccc6c5)cc5oc6ccccc6c45)n3)c2)cc1]

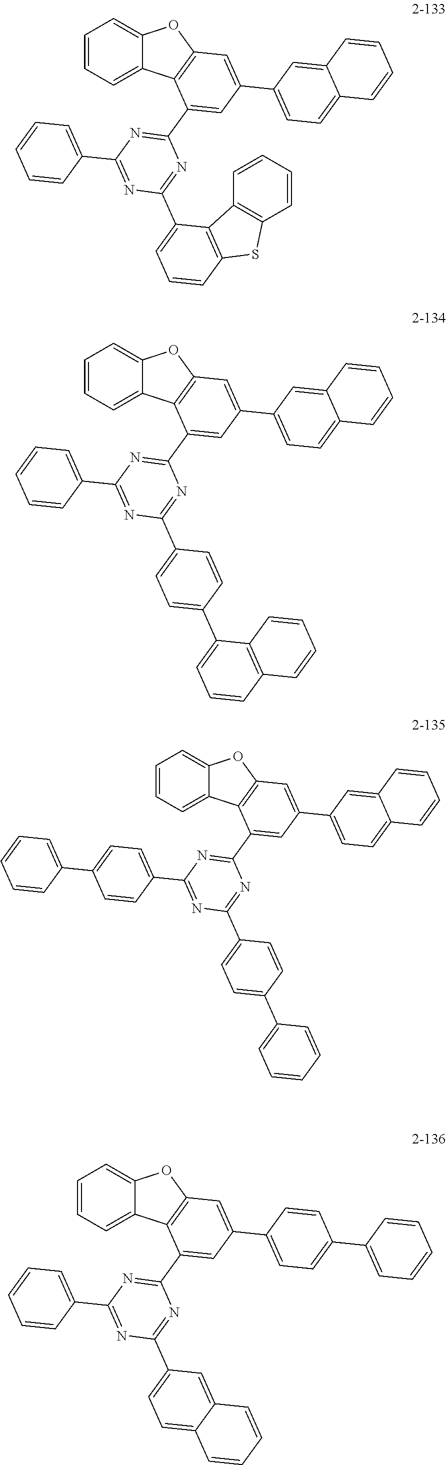 [SMILES: c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3cc(-c4nc(-c5ccccc5)nc(-c5ccc6ccccc6c5)n4)c4c(c3)oc3ccccc34)cc2)cc1]

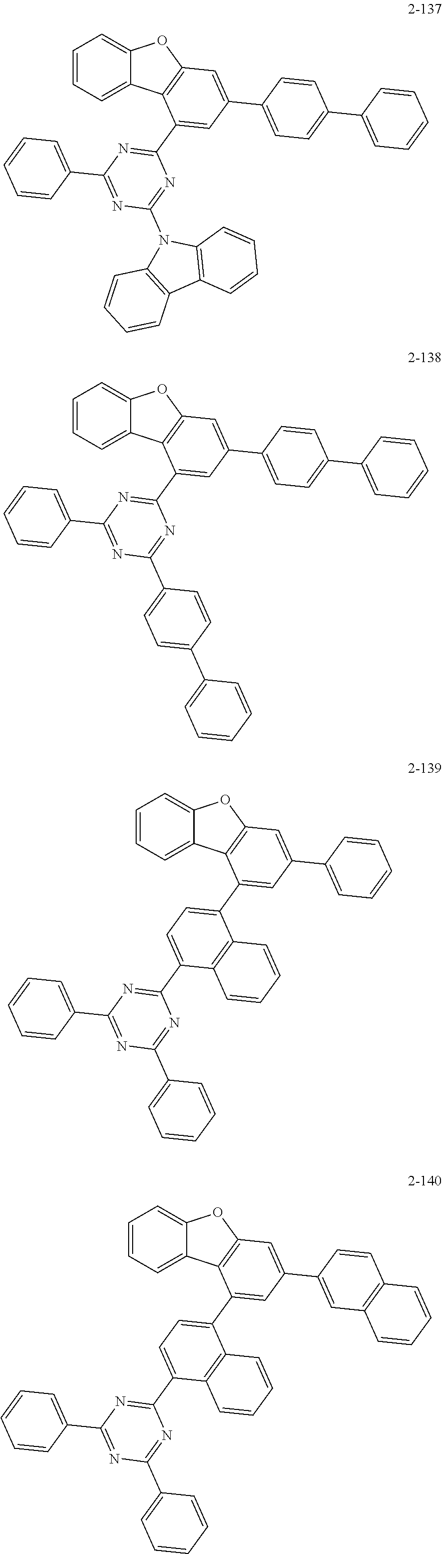 [SMILES: c1ccc(-c2ccc(-c3cc(-c4nc(-c5ccccc5)nc(-n5c6ccccc6c6ccccc65)n4)c4c(c3)oc3ccccc34)cc2)cc1 ; c1ccc(-c2ccc(-c3cc(-c4nc(-c5ccccc5)nc(-c5ccc(-c6ccccc6)cc5)n4)c4c(c3)oc3ccccc34)cc2)cc1 ; c1ccc(-c2cc(-c3ccc(-c4nc(-c5ccccc5)nc(-c5ccccc5)n4)c4ccccc34)c3c(c2)oc2ccccc23)cc1 ; c1ccc(-c2nc(-c3ccccc3)nc(-c3ccc(-c4cc(-c5ccc6ccccc6c5)cc5oc6ccccc6c45)c4ccccc34)n2)cc1]

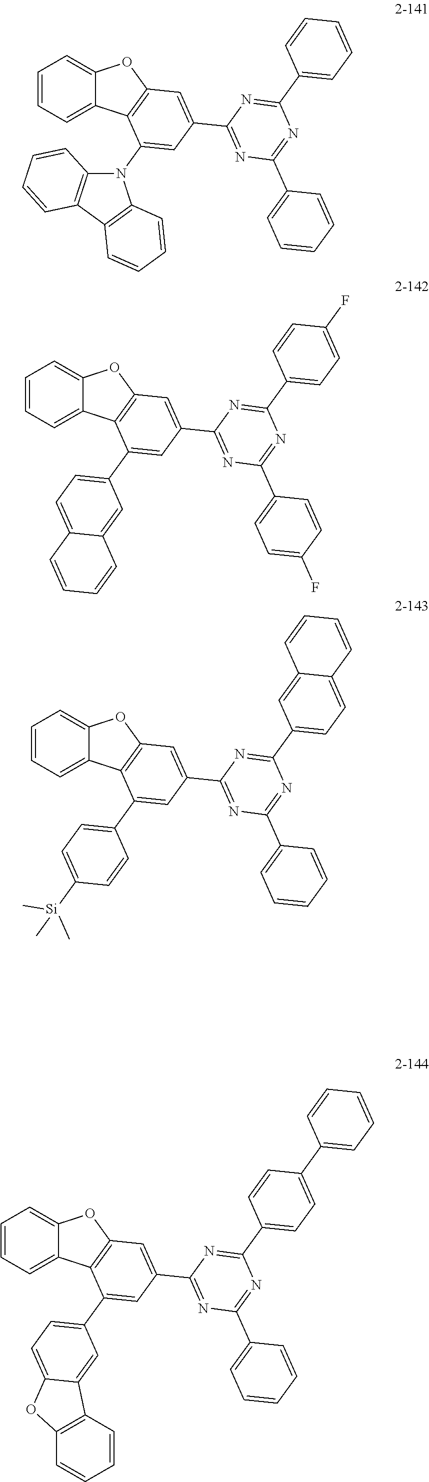

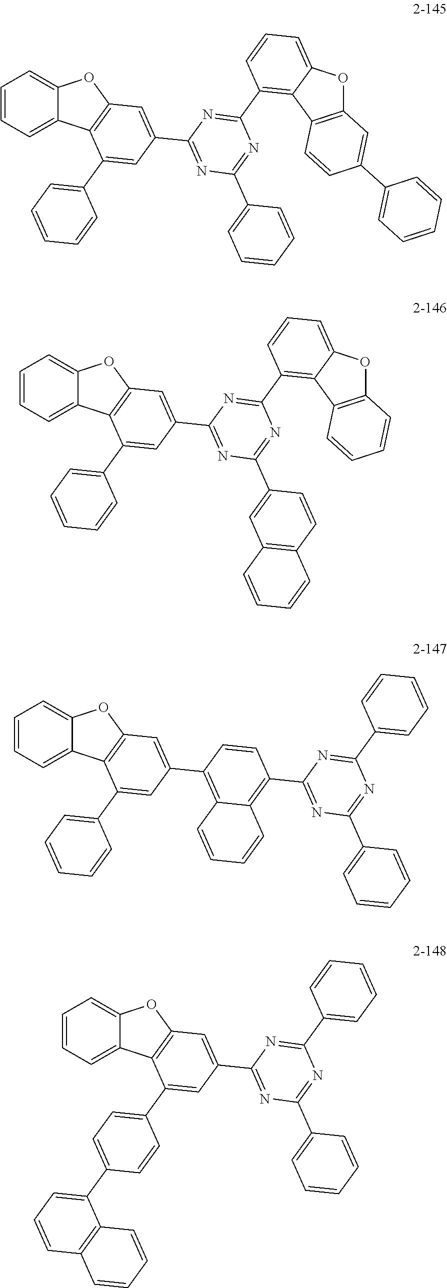 [SMILES: c1ccc(-c2ccc3c(c2)oc2cccc(-c4nc(-c5ccccc5)nc(-c5cc(-c6ccccc6)c6c(c5)oc5ccccc56)n4)c23)cc1 ; c1ccc(-c2cc(-c3nc(-c4ccc5ccccc5c4)nc(-c4cccc5oc6ccccc6c45)n3)cc3oc4ccccc4c23)cc1 ; c1ccc(-c2nc(-c3ccccc3)nc(-c3ccc(-c4cc(-c5ccccc5)c5c(c4)oc4ccccc45)c4ccccc34)n2)cc1 ; c1ccc(-c2nc(-c3ccccc3)nc(-c3cc(-c4ccc(-c5cccc6ccccc56)cc4)c4c(c3)oc3ccccc34)n2)cc1]

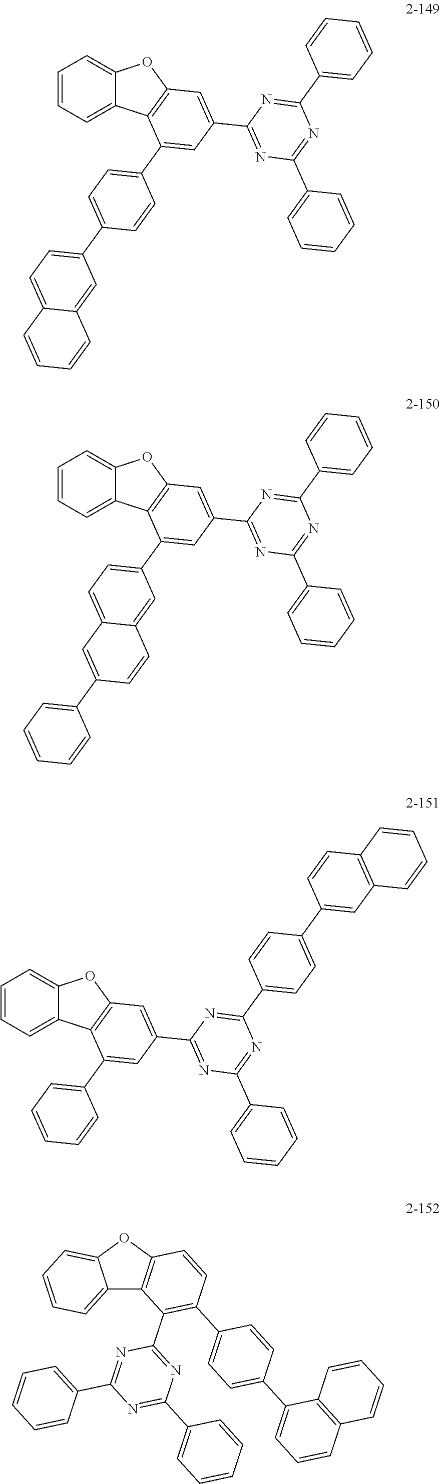 [SMILES: c1ccc(-c2nc(-c3ccccc3)nc(-c3cc(-c4ccc(-c5ccc6ccccc6c5)cc4)c4c(c3)oc3ccccc34)n2)cc1 ; c1ccc(-c2ccc3cc(-c4cc(-c5nc(-c6ccccc6)nc(-c6ccccc6)n5)cc5oc6ccccc6c45)ccc3c2)cc1 ; c1ccc(-c2nc(-c3ccc(-c4ccc5ccccc5c4)cc3)nc(-c3cc(-c4ccccc4)c4c(c3)oc3ccccc34)n2)cc1 ; c1ccc(-c2nc(-c3ccccc3)nc(-c3c(-c4ccc(-c5cccc6ccccc56)cc4)ccc4oc5ccccc5c34)n2)cc1]

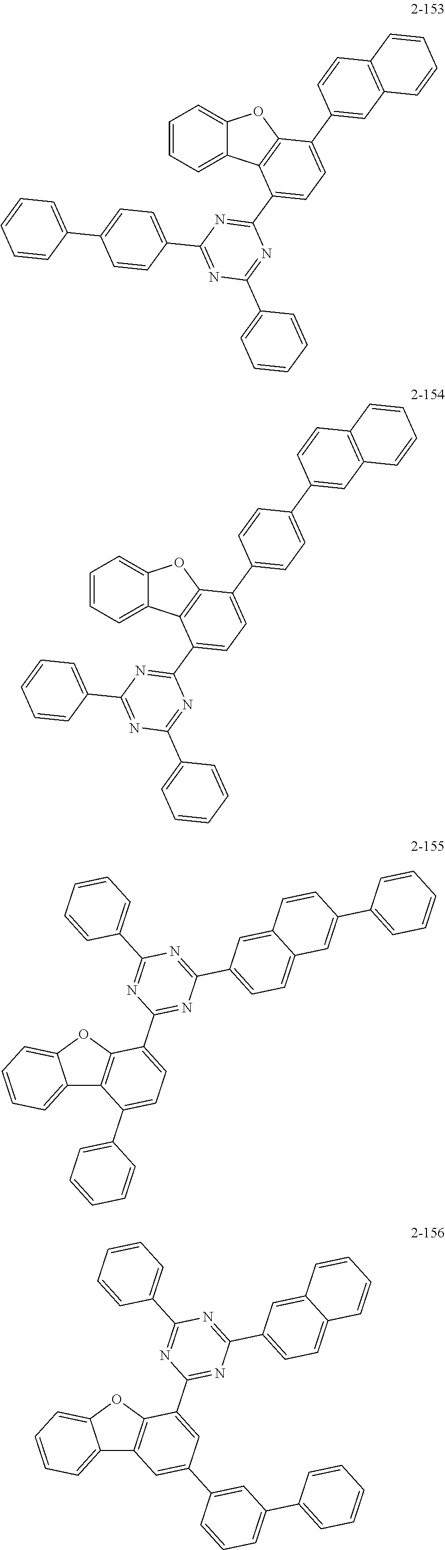 [SMILES: c1ccc(-c2ccc(-c3nc(-c4ccccc4)nc(-c4ccc(-c5ccc6ccccc6c5)c5oc6ccccc6c45)n3)cc2)cc1 ; c1ccc(-c2nc(-c3ccccc3)nc(-c3ccc(-c4ccc(-c5ccc6ccccc6c5)cc4)c4oc5ccccc5c34)n2)cc1 ; c1ccc(-c2ccc3cc(-c4nc(-c5ccccc5)nc(-c5ccc(-c6ccccc6)c6c5oc5ccccc56)n4)ccc3c2)cc1 ; c1ccc(-c2cccc(-c3cc(-c4nc(-c5ccccc5)nc(-c5ccc6ccccc6c5)n4)c4oc5ccccc5c4c3)c2)cc1]

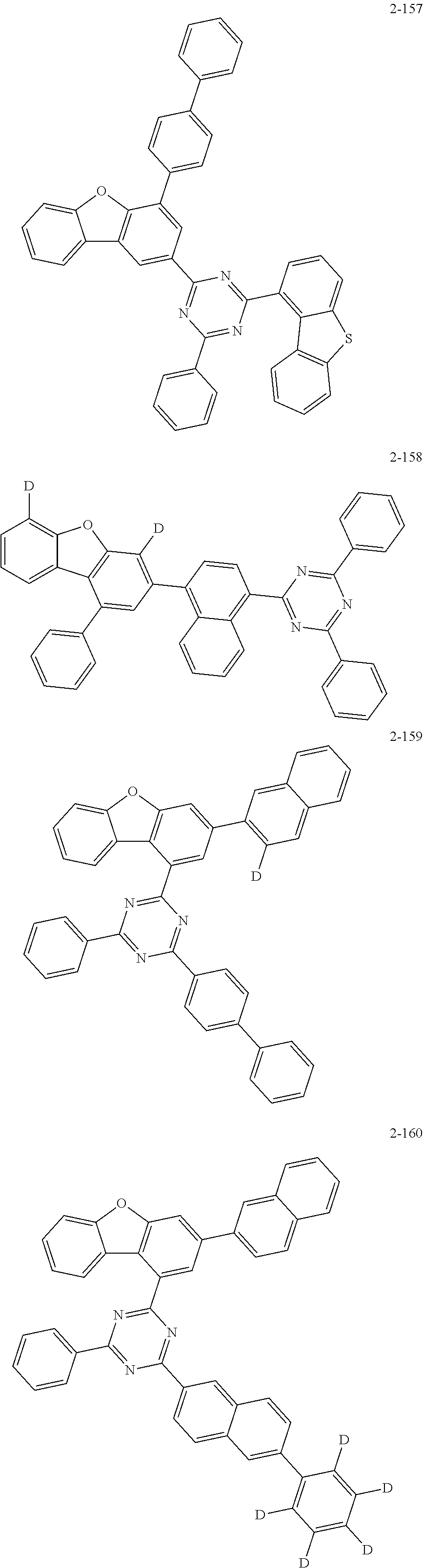

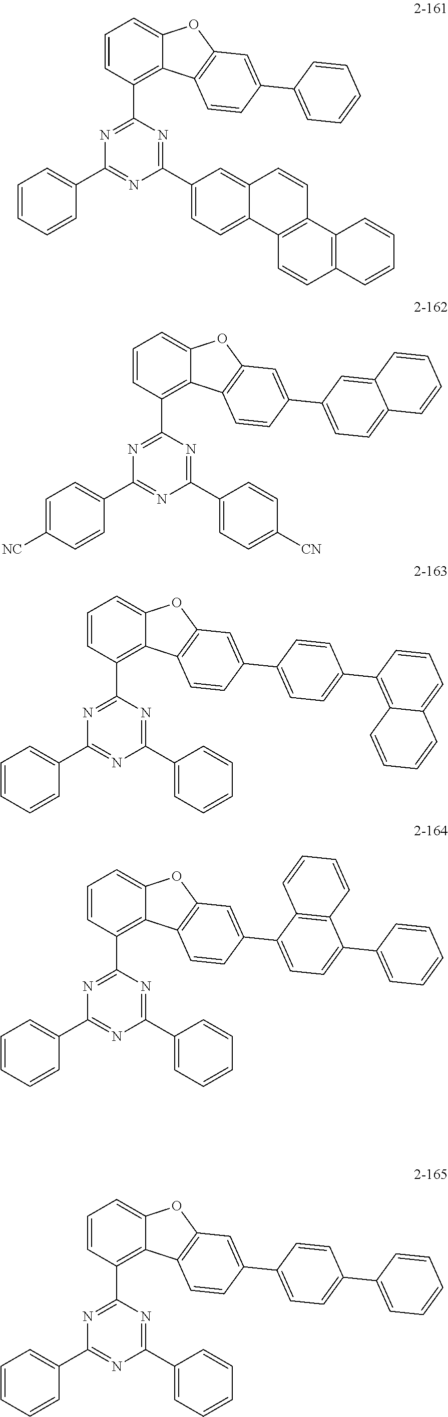 [SMILES: c1ccc(-c2ccc3c(c2)oc2cccc(-c4nc(-c5ccccc5)nc(-c5ccc6c(ccc7c8ccccc8ccc67)c5)n4)c23)cc1 ; N#Cc1ccc(-c2nc(-c3ccc(C#N)cc3)nc(-c3cccc4oc5cc(-c6ccc7ccccc7c6)ccc5c34)n2)cc1 ; c1ccc(-c2nc(-c3ccccc3)nc(-c3cccc4oc5cc(-c6ccc(-c7cccc8ccccc78)cc6)ccc5c34)n2)cc1 ; c1ccc(-c2nc(-c3ccccc3)nc(-c3cccc4oc5cc(-c6ccc(-c7ccccc7)c7ccccc67)ccc5c34)n2)cc1 ; c1ccc(-c2ccc(-c3ccc4c(c3)oc3cccc(-c5nc(-c6ccccc6)nc(-c6ccccc6)n5)c34)cc2)cc1]

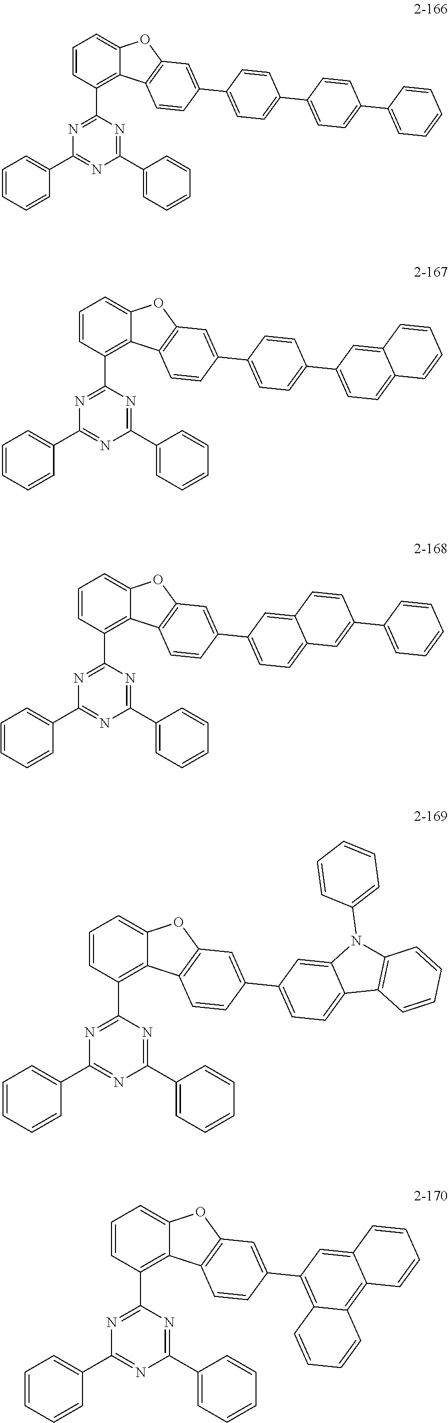 [SMILES: c1ccc(-c2ccc(-c3ccc(-c4ccc5c(c4)oc4cccc(-c6nc(-c7ccccc7)nc(-c7ccccc7)n6)c45)cc3)cc2)cc1 ; c1ccc(-c2nc(-c3ccccc3)nc(-c3cccc4oc5cc(-c6ccc(-c7ccc8ccccc8c7)cc6)ccc5c34)n2)cc1 ; c1ccc(-c2ccc3cc(-c4ccc5c(c4)oc4cccc(-c6nc(-c7ccccc7)nc(-c7ccccc7)n6)c45)ccc3c2)cc1 ; c1ccc(-c2nc(-c3ccccc3)nc(-c3cccc4oc5cc(-c6ccc7c8ccccc8n(-c8ccccc8)c7c6)ccc5c34)n2)cc1 ; c1ccc(-c2nc(-c3ccccc3)nc(-c3cccc4oc5cc(-c6cc7ccccc7c7ccccc67)ccc5c34)n2)cc1]

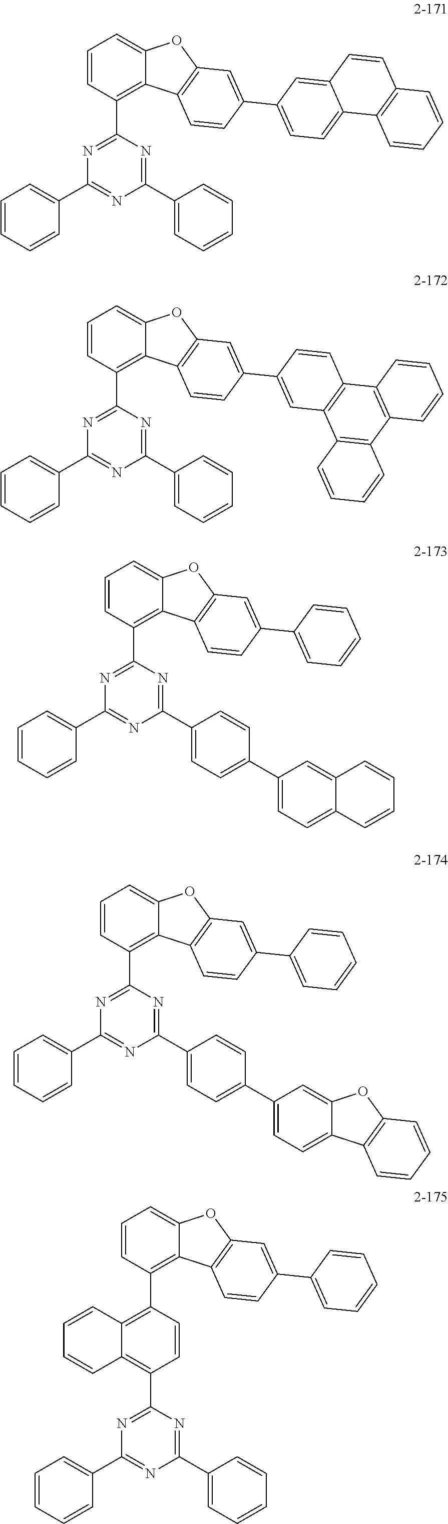

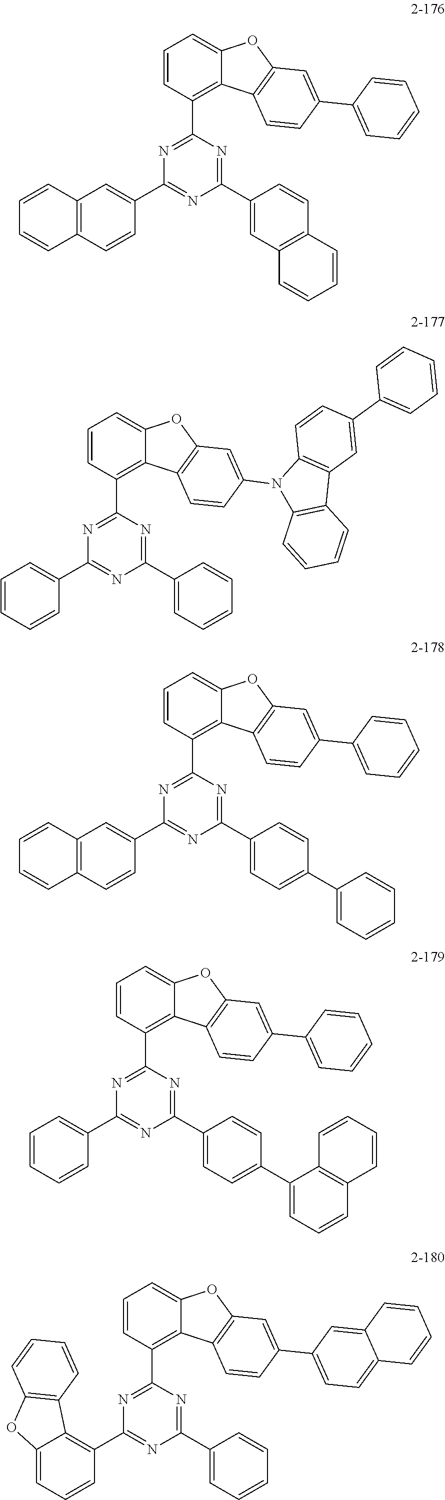

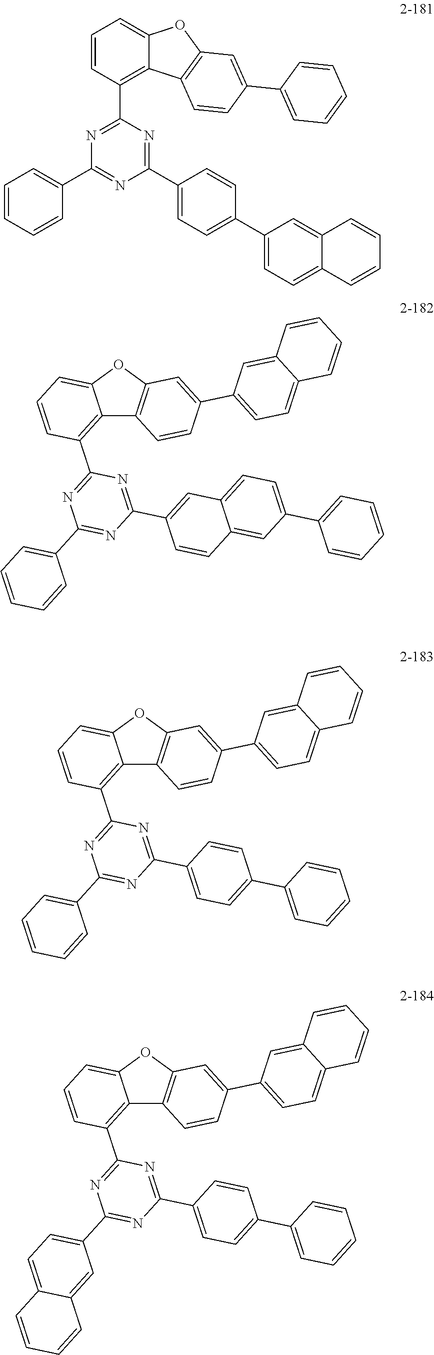

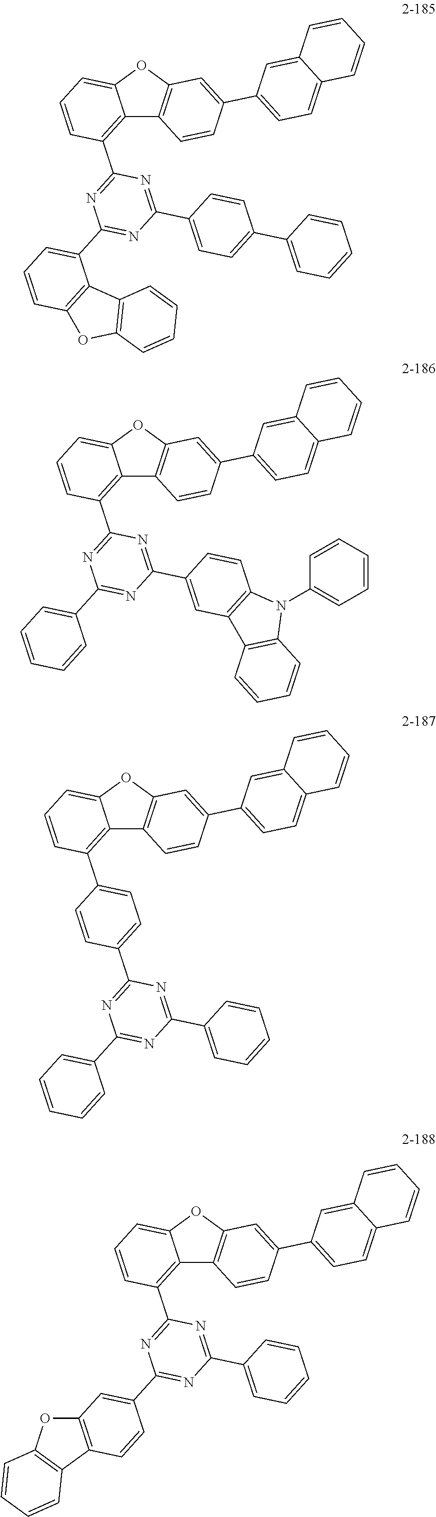

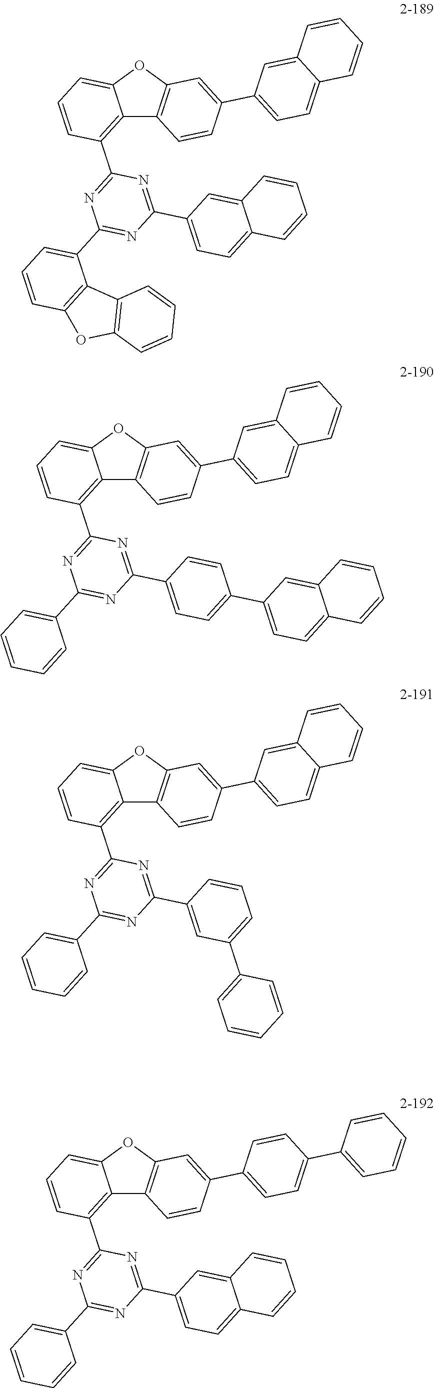 [SMILES: c1ccc2cc(-c3ccc4c(c3)oc3cccc(-c5nc(-c6ccc7ccccc7c6)nc(-c6cccc7oc8ccccc8c67)n5)c34)ccc2c1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2cccc(-c3nc(-c4ccccc4)nc(-c4cccc5oc6cc(-c7ccc8ccccc8c7)ccc6c45)n3)c2)cc1 ; c1ccc(-c2ccc(-c3ccc4c(c3)oc3cccc(-c5nc(-c6ccccc6)nc(-c6ccc7ccccc7c6)n5)c34)cc2)cc1]

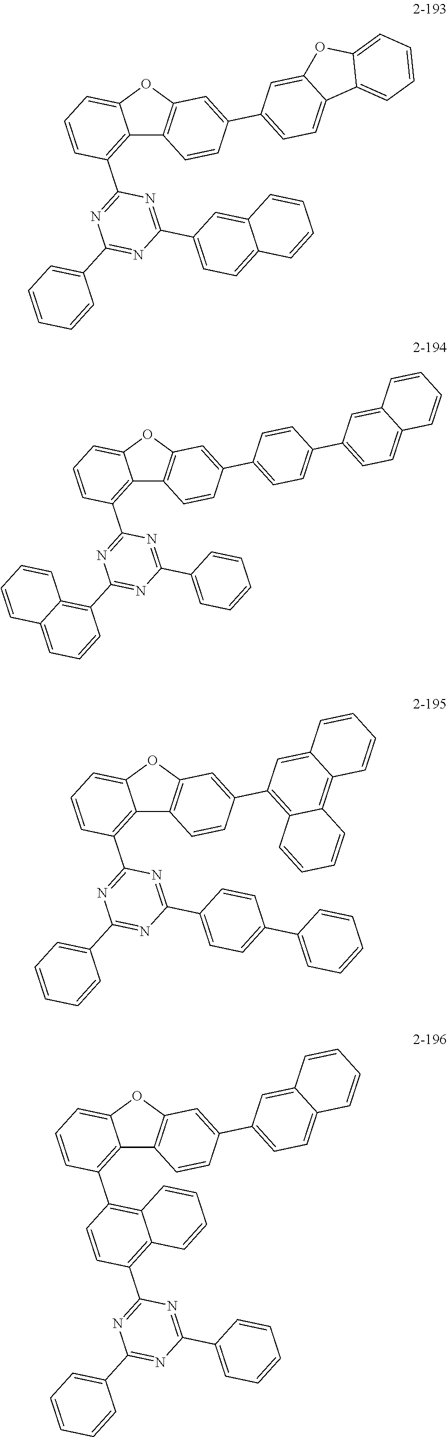 [SMILES: c1ccc(-c2nc(-c3ccc4ccccc4c3)nc(-c3cccc4oc5cc(-c6ccc7c(c6)oc6ccccc67)ccc5c34)n2)cc1 ; c1ccc(-c2nc(-c3cccc4ccccc34)nc(-c3cccc4oc5cc(-c6ccc(-c7ccc8ccccc8c7)cc6)ccc5c34)n2)cc1 ; c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2nc(-c3ccccc3)nc(-c3ccc(-c4cccc5oc6cc(-c7ccc8ccccc8c7)ccc6c45)c4ccccc34)n2)cc1]

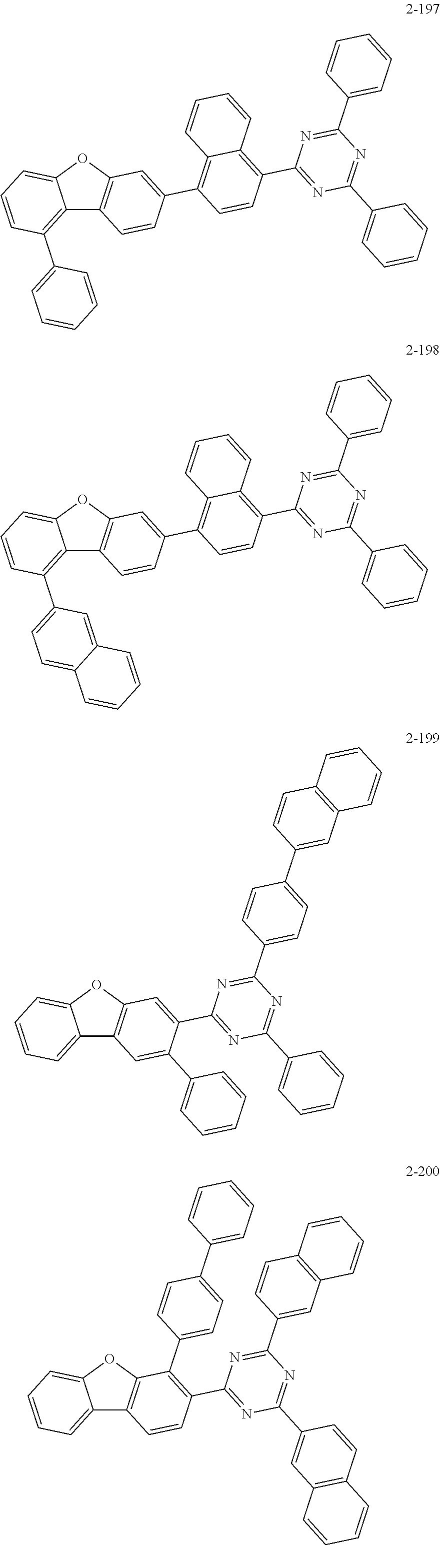 [SMILES: c1ccc(-c2nc(-c3ccccc3)nc(-c3ccc(-c4ccc5c(c4)oc4cccc(-c6ccccc6)c45)c4ccccc34)n2)cc1 ; c1ccc(-c2nc(-c3ccccc3)nc(-c3ccc(-c4ccc5c(c4)oc4cccc(-c6ccc7ccccc7c6)c45)c4ccccc34)n2)cc1 ; c1ccc(-c2nc(-c3ccc(-c4ccc5ccccc5c4)cc3)nc(-c3cc4oc5ccccc5c4cc3-c3ccccc3)n2)cc1 ; c1ccc(-c2ccc(-c3c(-c4nc(-c5ccc6ccccc6c5)nc(-c5ccc6ccccc6c5)n4)ccc4c3oc3ccccc34)cc2)cc1]

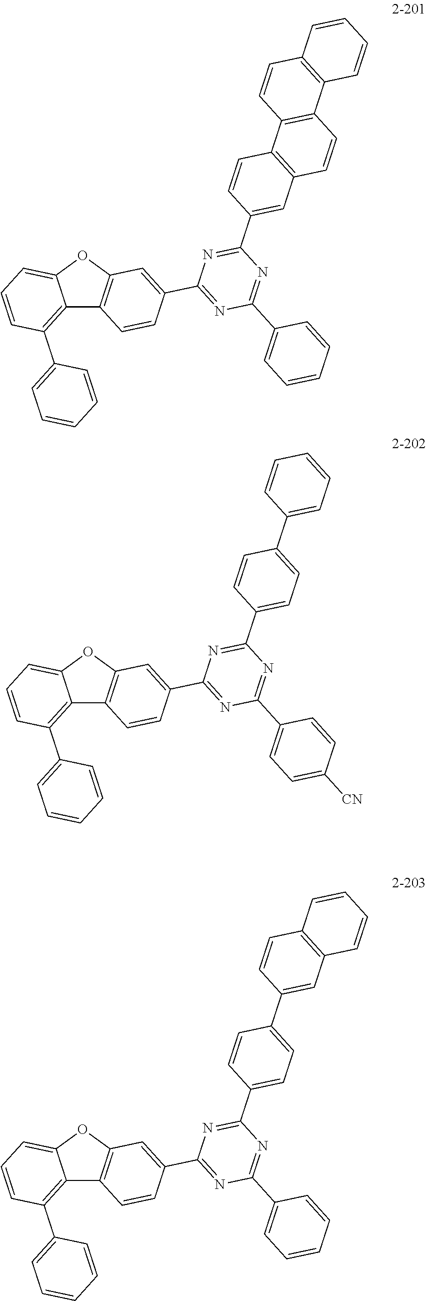 [SMILES: c1ccc(-c2nc(-c3ccc4c(ccc5c6ccccc6ccc45)c3)nc(-c3ccc4c(c3)oc3cccc(-c5ccccc5)c34)n2)cc1 ; N#Cc1ccc(-c2nc(-c3ccc(-c4ccccc4)cc3)nc(-c3ccc4c(c3)oc3cccc(-c5ccccc5)c34)n2)cc1 ; c1ccc(-c2nc(-c3ccc(-c4ccc5ccccc5c4)cc3)nc(-c3ccc4c(c3)oc3cccc(-c5ccccc5)c34)n2)cc1]

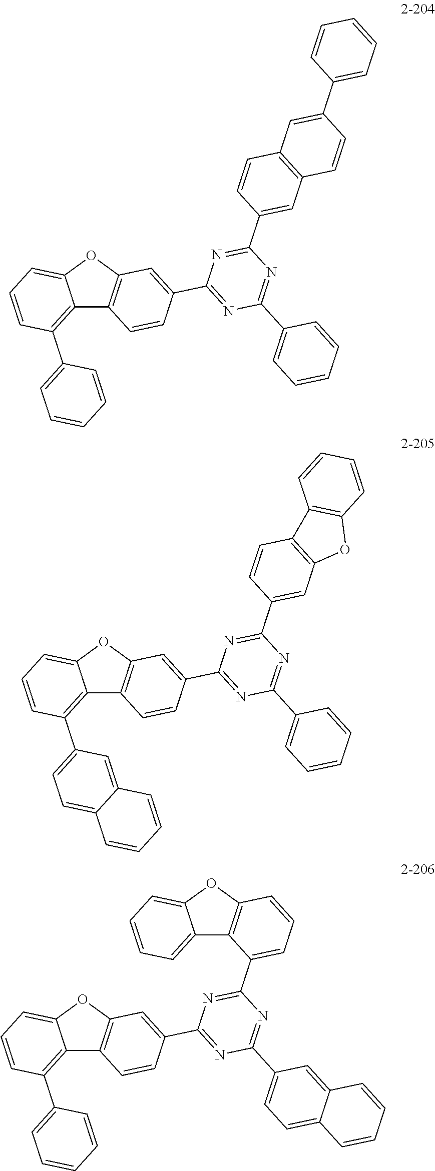

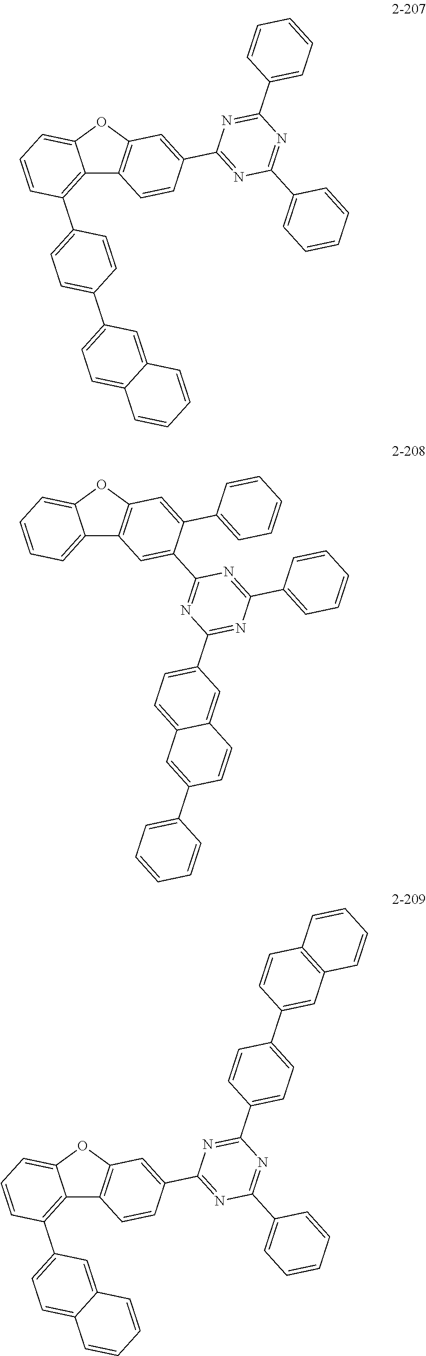 [SMILES: c1ccc(-c2nc(-c3ccccc3)nc(-c3ccc4c(c3)oc3cccc(-c5ccc(-c6ccc7ccccc7c6)cc5)c34)n2)cc1 ; c1ccc(-c2ccc3cc(-c4nc(-c5ccccc5)nc(-c5cc6c(cc5-c5ccccc5)oc5ccccc56)n4)ccc3c2)cc1 ; c1ccc(-c2nc(-c3ccc(-c4ccc5ccccc5c4)cc3)nc(-c3ccc4c(c3)oc3cccc(-c5ccc6ccccc6c5)c34)n2)cc1]

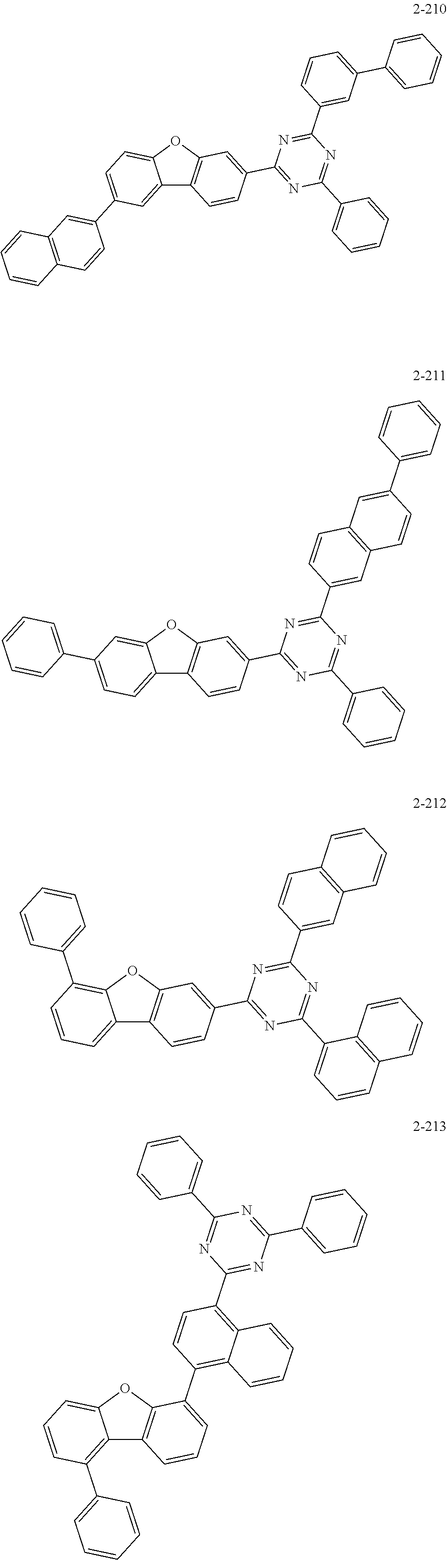 [SMILES: c1ccc(-c2cccc(-c3nc(-c4ccccc4)nc(-c4ccc5c(c4)oc4ccc(-c6ccc7ccccc7c6)cc45)n3)c2)cc1 ; c1ccc(-c2ccc3cc(-c4nc(-c5ccccc5)nc(-c5ccc6c(c5)oc5cc(-c7ccccc7)ccc56)n4)ccc3c2)cc1 ; c1ccc(-c2cccc3c2oc2cc(-c4nc(-c5ccc6ccccc6c5)nc(-c5cccc6ccccc56)n4)ccc23)cc1 ; c1ccc(-c2nc(-c3ccccc3)nc(-c3ccc(-c4cccc5c4oc4cccc(-c6ccccc6)c45)c4ccccc34)n2)cc1]

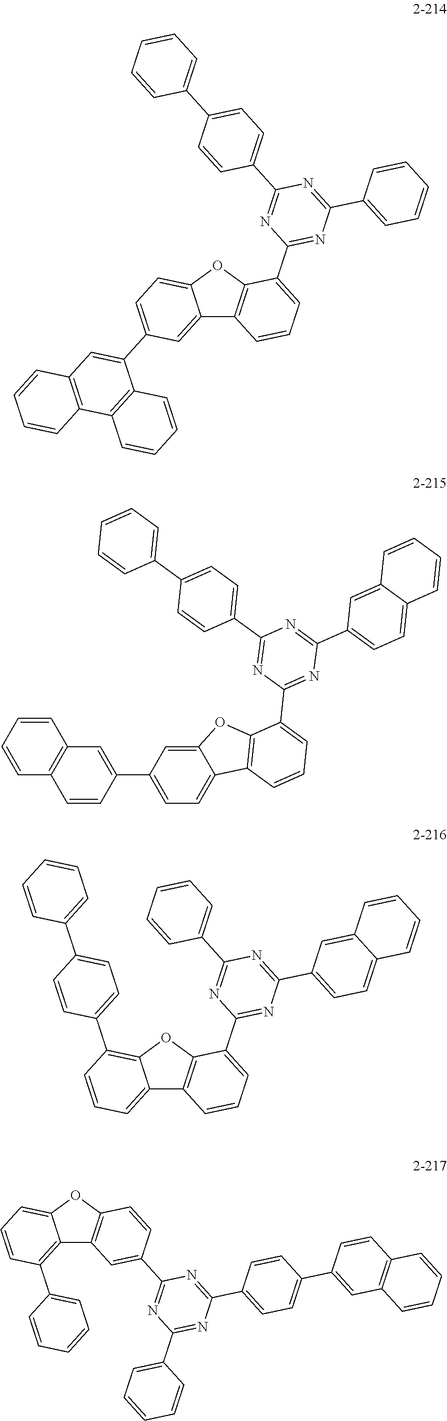 [SMILES: c1ccc(-c2ccc(-c3nc(-c4ccccc4)nc(-c4cccc5c4oc4ccc(-c6cc7ccccc7c7ccccc67)cc45)n3)cc2)cc1 ; c1ccc(-c2ccc(-c3nc(-c4ccc5ccccc5c4)nc(-c4cccc5c4oc4cc(-c6ccc7ccccc7c6)ccc45)n3)cc2)cc1 ; c1ccc(-c2ccc(-c3cccc4c3oc3c(-c5nc(-c6ccccc6)nc(-c6ccc7ccccc7c6)n5)cccc34)cc2)cc1 ; c1ccc(-c2nc(-c3ccc(-c4ccc5ccccc5c4)cc3)nc(-c3ccc4oc5cccc(-c6ccccc6)c5c4c3)n2)cc1]

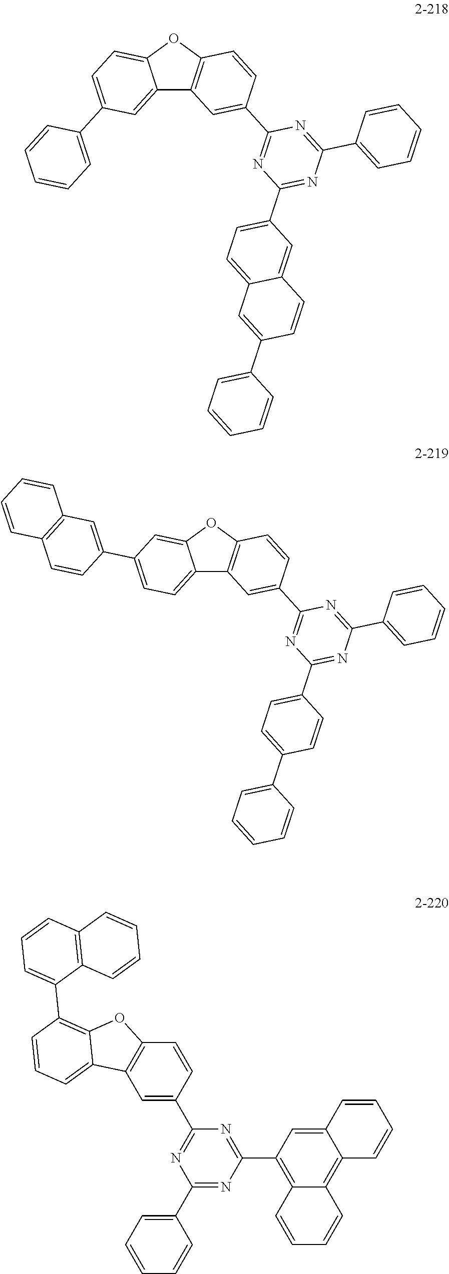 [SMILES: c1ccc(-c2ccc3cc(-c4nc(-c5ccccc5)nc(-c5ccc6oc7ccc(-c8ccccc8)cc7c6c5)n4)ccc3c2)cc1 ; c1ccc(-c2ccc(-c3nc(-c4ccccc4)nc(-c4ccc5oc6cc(-c7ccc8ccccc8c7)ccc6c5c4)n3)cc2)cc1 ; c1ccc(-c2nc(-c3ccc4oc5c(-c6cccc7ccccc67)cccc5c4c3)nc(-c3cc4ccccc4c4ccccc34)n2)cc1]

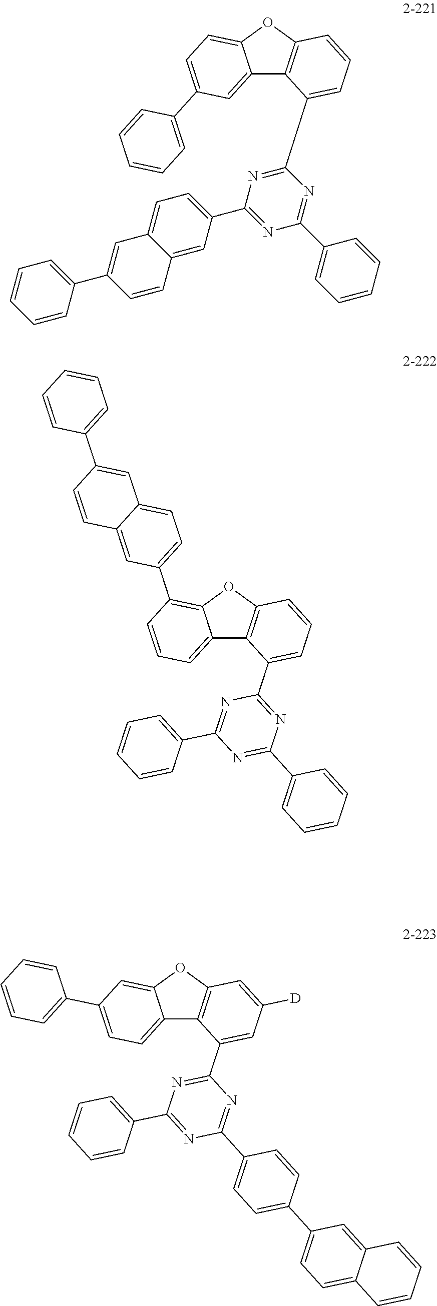 [SMILES: c1ccc(-c2ccc3cc(-c4nc(-c5ccccc5)nc(-c5cccc6oc7ccc(-c8ccccc8)cc7c56)n4)ccc3c2)cc1 ; c1ccc(-c2ccc3cc(-c4cccc5c4oc4cccc(-c6nc(-c7ccccc7)nc(-c7ccccc7)n6)c45)ccc3c2)cc1 ; [2H]c1cc(-c2nc(-c3ccccc3)nc(-c3ccc(-c4ccc5ccccc5c4)cc3)n2)c2c(c1)oc1cc(-c3ccccc3)ccc12]

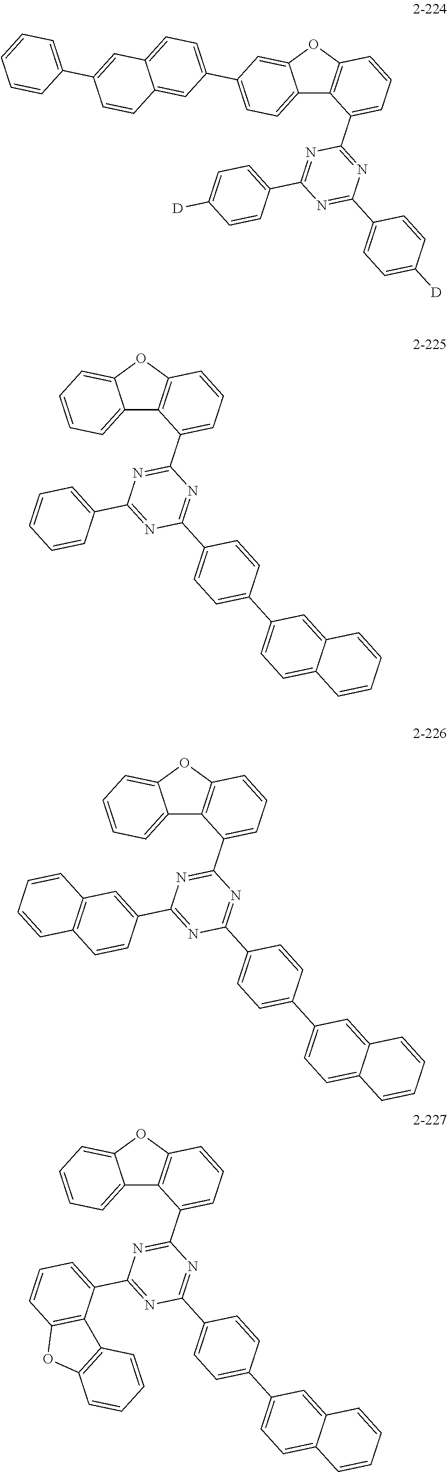 [SMILES: [2H]c1ccc(-c2nc(-c3ccc([2H])cc3)nc(-c3cccc4oc5cc(-c6ccc7cc(-c8ccccc8)ccc7c6)ccc5c34)n2)cc1 ; c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc2cc(-c3ccc(-c4nc(-c5cccc6oc7ccccc7c56)nc(-c5cccc6oc7ccccc7c56)n4)cc3)ccc2c1]

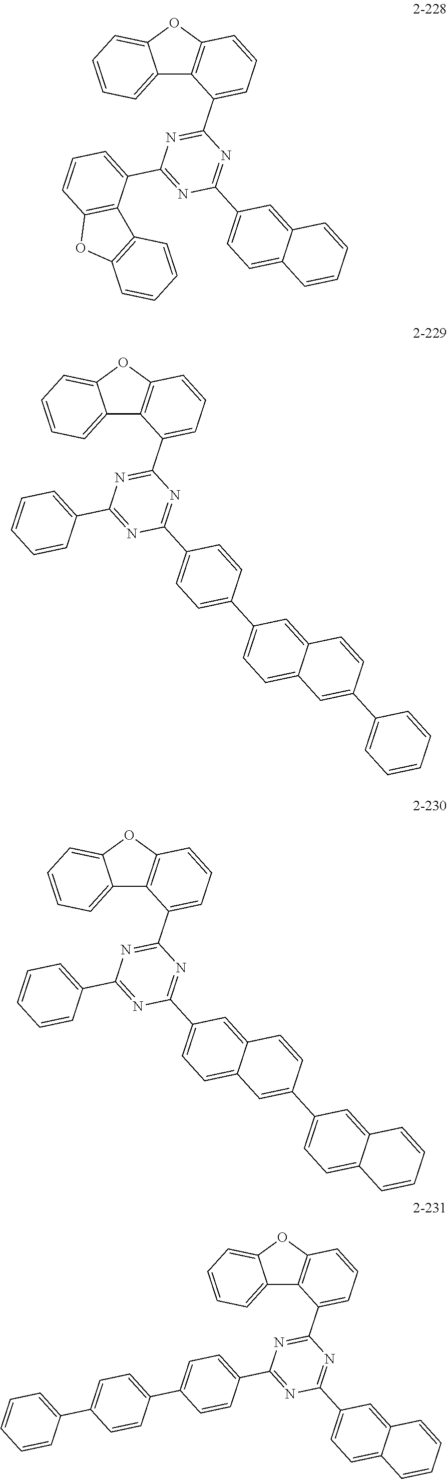 [SMILES: c1ccc2cc(-c3nc(-c4cccc5oc6ccccc6c45)nc(-c4cccc5oc6ccccc6c45)n3)ccc2c1 ; c1ccc(-c2ccc3cc(-c4ccc(-c5nc(-c6ccccc6)nc(-c6cccc7oc8ccccc8c67)n5)cc4)ccc3c2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1]

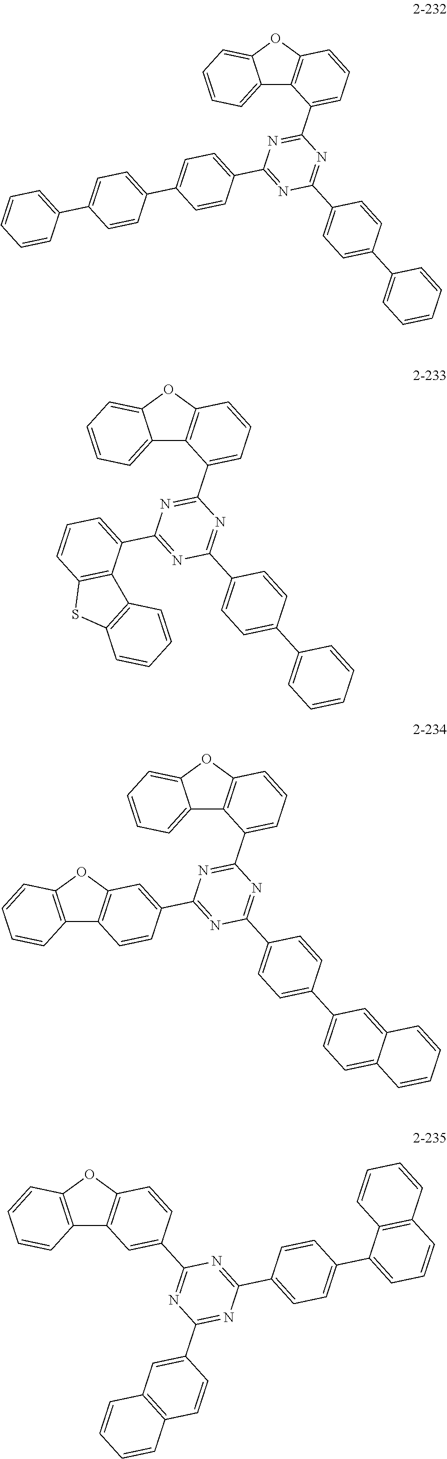 [SMILES: c1ccc(-c2ccc(-c3ccc(-c4nc(-c5ccc(-c6ccccc6)cc5)nc(-c5cccc6oc7ccccc7c56)n4)cc3)cc2)cc1 ; c1ccc(-c2ccc(-c3nc(-c4cccc5oc6ccccc6c45)nc(-c4cccc5sc6ccccc6c45)n3)cc2)cc1 ; c1ccc2cc(-c3ccc(-c4nc(-c5ccc6c(c5)oc5ccccc56)nc(-c5cccc6oc7ccccc7c56)n4)cc3)ccc2c1 ; c1ccc2cc(-c3nc(-c4ccc(-c5cccc6ccccc56)cc4)nc(-c4ccc5oc6ccccc6c5c4)n3)ccc2c1]

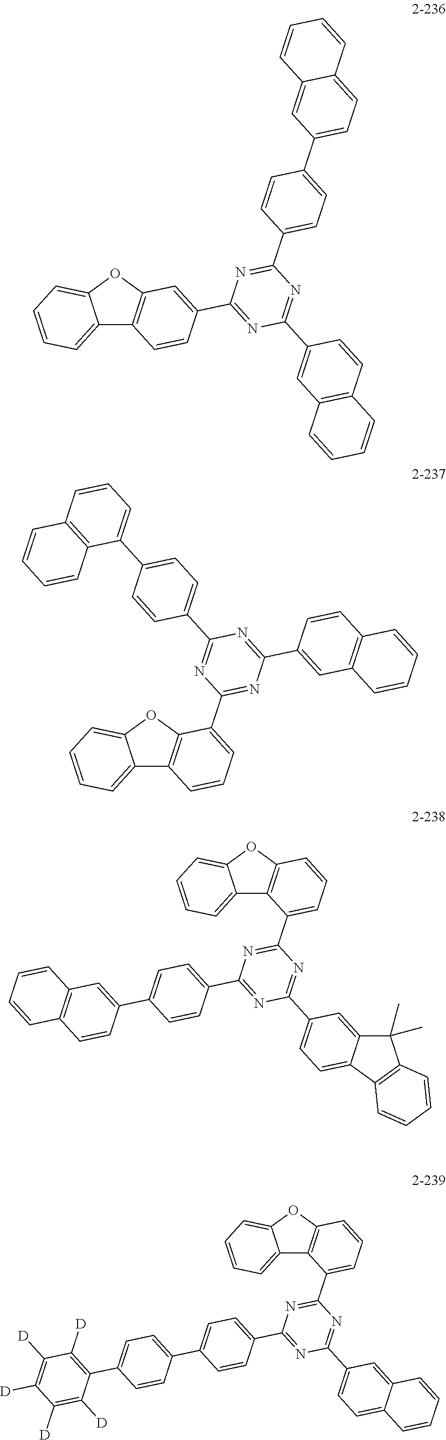 [SMILES: c1ccc2cc(-c3ccc(-c4nc(-c5ccc6ccccc6c5)nc(-c5ccc6c(c5)oc5ccccc56)n4)cc3)ccc2c1 ; c1ccc2cc(-c3nc(-c4ccc(-c5cccc6ccccc56)cc4)nc(-c4cccc5c4oc4ccccc45)n3)ccc2c1 ; CC1(C)c2ccccc2-c2ccc(-c3nc(-c4ccc(-c5ccc6ccccc6c5)cc4)nc(-c4cccc5oc6ccccc6c45)n3)cc21 ; [2H]c1c([2H])c([2H])c(-c2ccc(-c3ccc(-c4nc(-c5ccc6ccccc6c5)nc(-c5cccc6oc7ccccc7c56)n4)cc3)cc2)c([2H])c1[2H]]

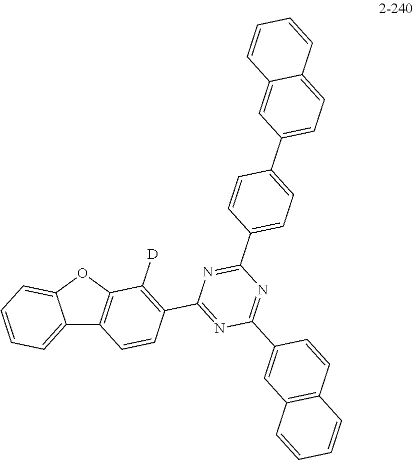 [SMILES: [2H]c1c(-c2nc(-c3ccc(-c4ccc5ccccc5c4)cc3)nc(-c3ccc4ccccc4c3)n2)ccc2c1oc1ccccc12]

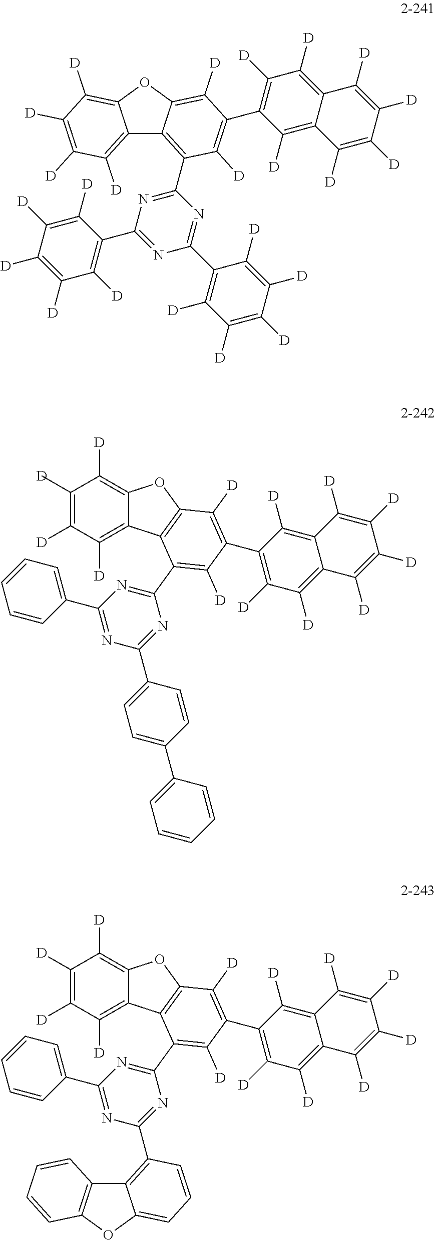

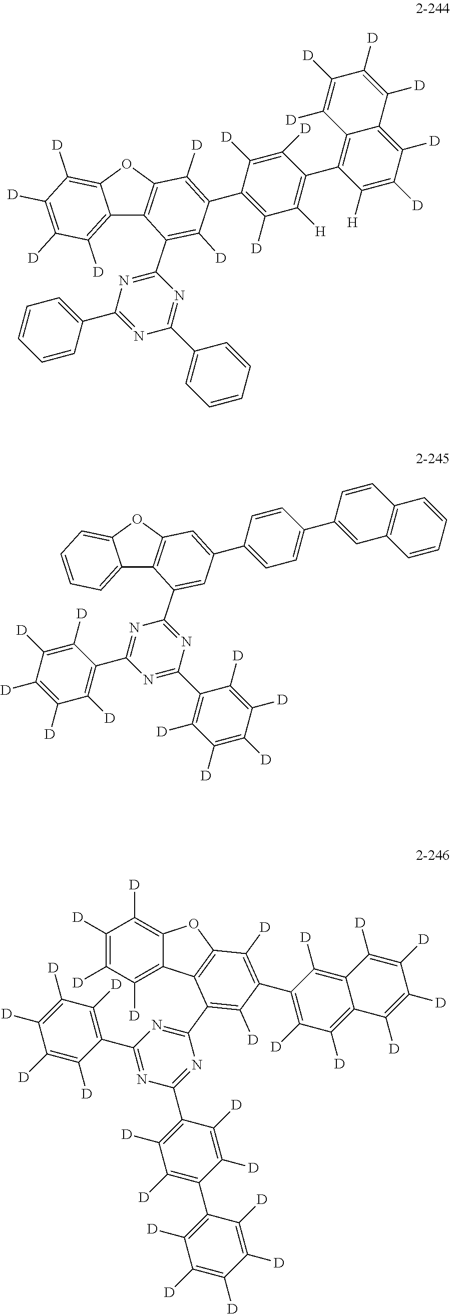

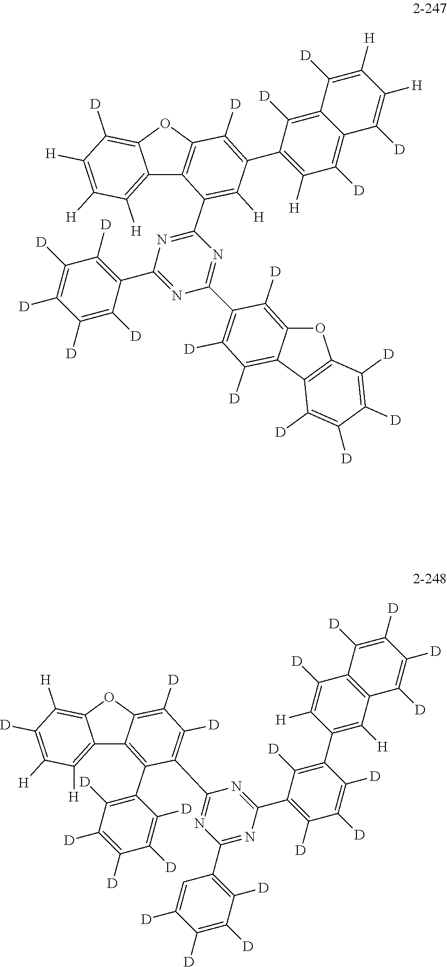 [SMILES: [2H]c1c([2H])c([2H])c(-c2nc(-c3c([2H])c([2H])c4c(oc5c([2H])c([2H])c([2H])c([2H])c54)c3[2H])nc(-c3cc(-c4cc([2H])c5c([2H])ccc([2H])c5c4[2H])c([2H])c4oc5c([2H])cccc5c34)n2)c([2H])c1[2H] ; [2H]c1ccc2c(c1)oc1c([2H])c([2H])c(-c3nc(-c4cc([2H])c([2H])c([2H])c4[2H])nc(-c4c([2H])c([2H])c([2H])c(-c5cc([2H])c6c([2H])c([2H])c([2H])c([2H])c6c5)c4[2H])n3)c(-c3c([2H])c([2H])c([2H])c([2H])c3[2H])c12]

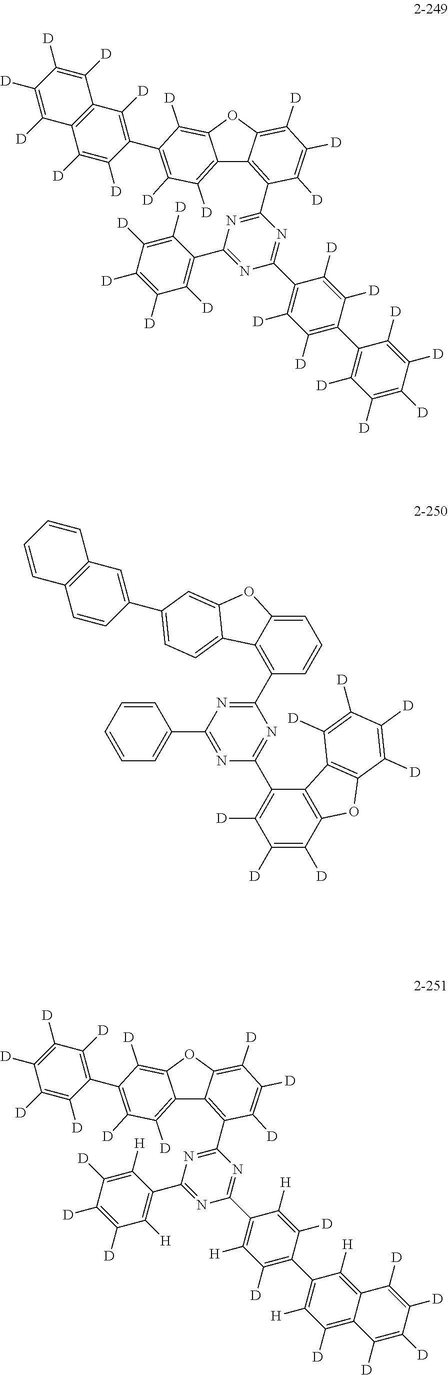

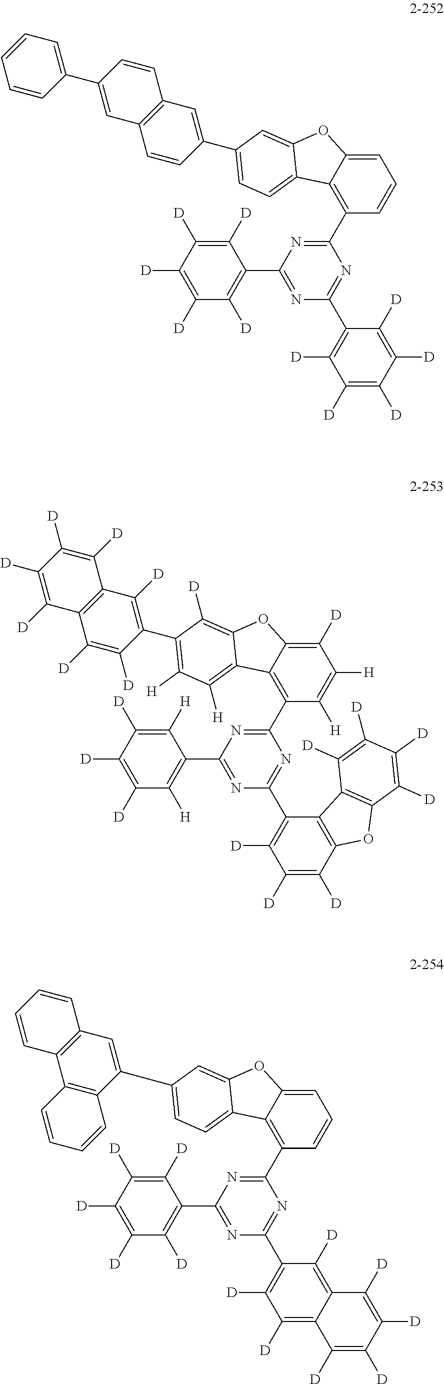 [SMILES: [2H]c1c([2H])c([2H])c(-c2nc(-c3c([2H])c([2H])c([2H])c([2H])c3[2H])nc(-c3cccc4oc5cc(-c6ccc7cc(-c8ccccc8)ccc7c6)ccc5c34)n2)c([2H])c1[2H] ; [2H]c1cc(-c2nc(-c3ccc([2H])c4oc5c([2H])c(-c6c([2H])c([2H])c7c([2H])c([2H])c([2H])c([2H])c7c6[2H])ccc5c34)nc(-c3c([2H])c([2H])c([2H])c4oc5c([2H])c([2H])c([2H])c([2H])c5c34)n2)cc([2H])c1[2H] ; [2H]c1c([2H])c([2H])c(-c2nc(-c3c([2H])c([2H])c4c([2H])c([2H])c([2H])c([2H])c4c3[2H])nc(-c3cccc4oc5cc(-c6cc7ccccc7c7ccccc67)ccc5c34)n2)c([2H])c1[2H]]

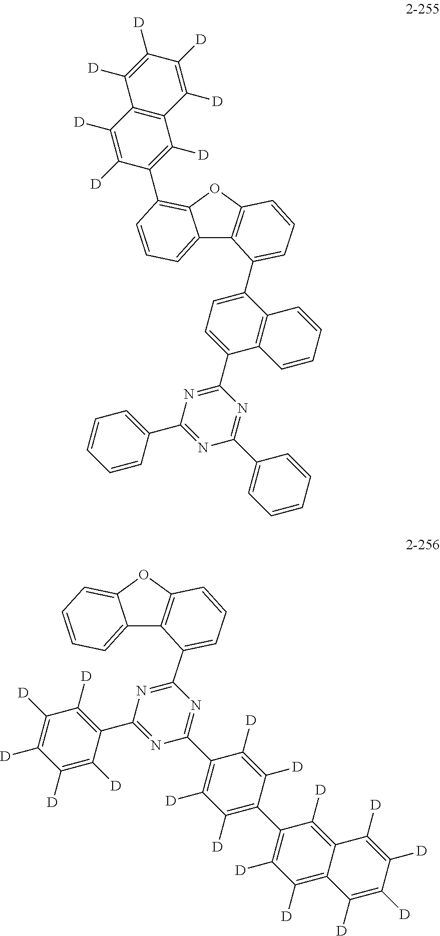 [SMILES: [2H]c1c([2H])c([2H])c2c([2H])c(-c3cccc4c3oc3cccc(-c5ccc(-c6nc(-c7ccccc7)nc(-c7ccccc7)n6)c6ccccc56)c34)c([2H])c([2H])c2c1[2H] ; [2H]c1c([2H])c([2H])c(-c2nc(-c3c([2H])c([2H])c(-c4c([2H])c([2H])c5c([2H])c([2H])c([2H])c([2H])c5c4[2H])c([2H])c3[2H])nc(-c3cccc4oc5ccccc5c34)n2)c([2H])c1[2H]]

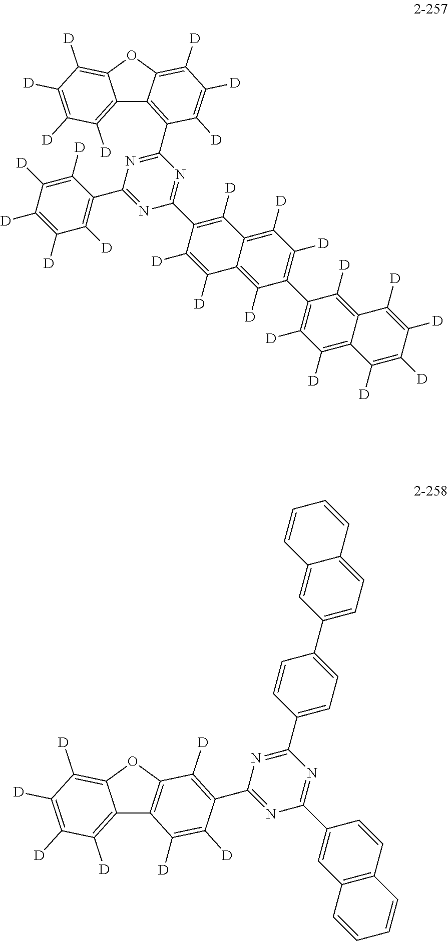 [SMILES: [2H]c1c([2H])c([2H])c(-c2nc(-c3c([2H])c([2H])c4c([2H])c(-c5c([2H])c([2H])c6c([2H])c([2H])c([2H])c([2H])c6c5[2H])c([2H])c([2H])c4c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c2c(oc3c([2H])c(-c4nc(-c5ccc(-c6ccc7ccccc7c6)cc5)nc(-c5ccc6ccccc6c5)n4)c([2H])c([2H])c32)c1[2H]]

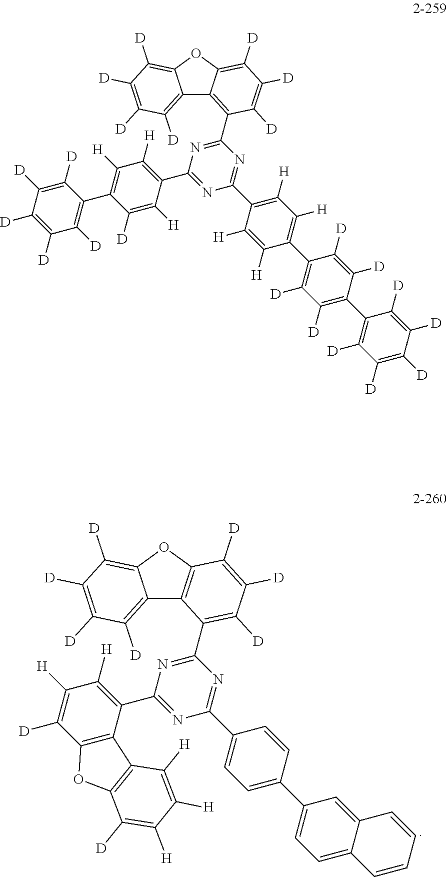 [SMILES: [2H]c1cc(-c2nc(-c3ccc(-c4c([2H])c([2H])c(-c5c([2H])c([2H])c([2H])c([2H])c5[2H])c([2H])c4[2H])cc3)nc(-c3c([2H])c([2H])c([2H])c4oc5c([2H])c([2H])c([2H])c([2H])c5c34)n2)ccc1-c1c([2H])c([2H])c([2H])c([2H])c1[2H] ; [2H]c1c([2H])c([2H])c2c(oc3c([2H])c([2H])c([2H])c(-c4nc(-c5ccc(-c6ccc7ccccc7c6)cc5)nc(-c5ccc([2H])c6oc7c([2H])cccc7c56)n4)c32)c1[2H]]

15. A composition for an organic layer of an organic light-emitting device, comprising:
the heterocyclic compound represented by Formula 1 according to claim 1; and
a compound represented by Formula 3 below: 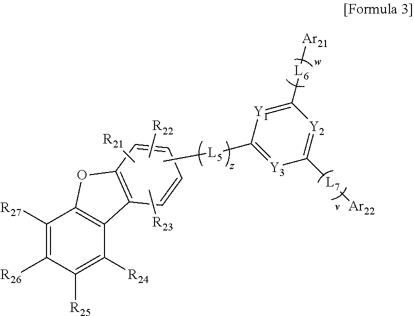
wherein, in Formula 3, any one of R₂₁ to R₂₇ is a substituted or unsubstituted aryl group having 6 to 60 carbon atoms, or a substituted or unsubstituted heteroaryl group having 2 to 60 carbon atoms,
the others of R₂₁ to R₂₇ are each independently selected from the group consisting of hydrogen, deuterium, a halogen, a cyano group, a substituted or unsubstituted alkyl group having 1 to 60 carbon atoms, a substituted or unsubstituted alkenyl group having 2 to 60 carbon atoms, a substituted or unsubstituted alkynyl group having 2 to 60 carbon atoms, a substituted or unsubstituted alkoxy group having 1 to 60 carbon atoms, a substituted or unsubstituted cycloalkyl group having 3 to 60 carbon atoms, a substituted or unsubstituted heterocycloalkyl group having 2 to 60 carbon atoms, a substituted or unsubstituted aryl group having 6 to 60 carbon atoms, a substituted or unsubstituted heteroaryl group having 2 to 60 carbon atoms, a phosphine oxide group, and a silyl group, or two or more adjacent groups are linked to form a substituted or unsubstituted aromatic hydrocarbon ring having 6 to 60 carbon atoms, or a substituted or unsubstituted heterocycle having 2 to 60 carbon atoms,
L₅, L₆ and L₇ are each independently a direct linkage, a substituted or unsubstituted arylene group having 6 to 60 carbon atoms, or a substituted or unsubstituted heteroarylene group having 2 to 60 carbon atoms,
w, v and z are each independently 0 or an integer of 1 to 5,
when w, v, and z are integers of 2 to 5, a plurality of L₅ groups, L₆ groups, and L₇ groups are each the same or different,
Y₁ to Y₃ are each independently CH, nitrogen (N), oxygen (O), or sulfur (S), and at least two of Y₁ to Y₃ are nitrogen, and
Ar₂₁ and Ar₂₂ are each independently a substituted or unsubstituted aryl group having 6 to 60 carbon atoms, or a substituted or unsubstituted heteroaryl group having 2 to 60 carbon atoms.

16. The composition for an organic layer of an organic light-emitting device according to claim 15, wherein a ratio of the weight of the compound represented by Formula 3 to the weight of the heterocyclic compound represented by Formula 1 in the total weight of the composition is 0.1 to 10.
